(19) United States
(12) Patent Application Publication
(10) Pub. No.: US 2023/0287409 A1
(43) Pub. Date: Sep. 14, 2023

(54) COMPOSITIONS AND METHODS FOR TREATING NEURODEGENERATIVE DISEASES

(71) Applicant: MAZE THERAPEUTICS, INC., South San Francisco, CA (US)

(72) Inventors: Carleton Proctor GOOLD, South San Francisco, CA (US); Ronald CHEN, Pacifica, CA (US); Peter JANKI, South San Francisco, CA (US); Eric GREEN, South San Francisco, CA (US)

(21) Appl. No.: 17/796,563

(22) PCT Filed: Feb. 5, 2021
(86) PCT No.: PCT/US2021/016939

§ 371 Date: Jul. 29, 2022

Related U.S. Application Data
(60) Provisional application No. 62/971,873, filed on Feb. 7, 2020.

(51) Int. Cl. C12N 15/113; A61P 25/28

(52) U.S. Cl. CPC C12N 15/113; A61P 25/28; C12N 2310/14; C12N 2310/141

(57) ABSTRACT

The invention relates to inhibitory nucleic acids targeting the ataxin-2 gene (ATXN2), and expression cassettes and vectors comprising the same. Also provided herein are methods of treating neurodegenerative diseases, e.g., Amyotrophic Lateral Sclerosis and Spinocerebellar Ataxia-2.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 630264_401USPC_SEQUENCE_LISTING.txt. The text file is 666,463 bytes, was created on Jan. 6, 2023, and is being submitted electronically via EFS-Web.

### BACKGROUND

Ataxin-2 (ATXN2) protein is a cytoplasmic protein that is a component of stress granules. Stress granules are thought to be transient subcellular compartments induced by arrest of protein translation, and include a number of proteins known to be mutated in subjects with neurodegenerative disease (Brown and Al-Chalabi, N Engl J Med (2017) 377:162-172). Ataxin-2 contains a sequence of glutamine residues, known as a polyglutamine repeat, that in normal individuals is ˜22 amino acids in length. Expansions of this polyglutamine repeat to a length of 34 or longer is found in individuals with a neurodegenerative disease Spinocerebellar Ataxia-2 (SCA2). This disease is characterized by progressive death of Purkinje neurons in the cerebellum and other neuronal cell types. Patients with Spinocerebellar Ataxia-2 develop ataxia, sensory problems, and other clinical features, which worsen over time. Moderate expansion of Ataxin-2 polyglutamine repeat, which are longer than that observed in most individuals but that are shorter than those typically observed in subjects with Spinocerebellar Ataxia-2 (e.g., between 27 and 33 glutamine residues), have been reported at a substantially elevated frequency in individuals with the motor neuron disease amyotrophic lateral sclerosis (ALS) as compared to normal subjects (Elden et al., Nature (2010) 466:7310). This suggests that these polyglutamine repeats of intermediate length, i.e., between those found in normal individuals and those found in spinocerebellar ataxia-2 patients, increase risk for ALS. Currently, treatment options for SCA2 and ALS are limited.

### BRIEF SUMMARY

Aspects of the disclosure relate to compositions and methods for modulating expression of genes associated with spinocerebellar ataxia-2 (SCA2), amyotrophic lateral sclerosis (ALS), and conditions associated with TDP-43 proteinopathies. In particular, inhibitory nucleic acids are provided that are useful for inhibiting expression or activity of ataxin 2 (ATXN2). For example, inhibitory nucleic acids are provided that target one or more isoforms of ATXN2, e.g., a subset of ATXN2 isoforms, or all ATXN2 isoforms.

In one aspect, the disclosure provides an isolated nucleic acid molecule comprising an expression construct encoding an inhibitory nucleic acid that inhibits expression or activity of ATXN2, wherein the inhibitory nucleic acid comprises a guide strand sequence comprising the nucleic acid sequence set forth in any one of SEQ ID NOS: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 1176-1288, 1811-1827, 2015, 2065, 2083, 2152, 2203, and 2209.

In some embodiments, the inhibitory nucleic acid is a siRNA duplex, shRNA, miRNA, or dsRNA.

In some embodiments, the inhibitory nucleic acid further comprises a passenger strand sequence, optionally wherein the passenger strand sequence is selected from Tables 1, 19, 23, and 24, or a passenger strand sequence selected from Tables 1, 19, 23, and 24, and having 1-10 insertions, deletions, substitutions, mismatches, wobbles, or any combination thereof.

In some embodiments, the inhibitory nucleic acid is an artificial miRNA wherein the guide strand sequence is contained within a miRNA backbone sequence.

In some embodiments, the guide strand sequence and passenger strand sequence of the artificial miRNA are contained within a miRNA backbone sequence. In some embodiments, the miRNA backbone sequence is a miR-155 backbone sequence, a miR-155E backbone sequence, a miR-155M backbone sequence, miR1-1 backbone sequence, a miR-1-1_M backbone sequence, a miR-100 backbone sequence, a miR-100_M backbone sequence, a miR-190a backbone sequence, a miR-190a_M backbone sequence, a miR-124 backbone sequence, a miR-124_M backbone sequence, a miR-132 backbone sequence, a miR-9 backbone sequence, a miR-138-2 backbone sequence, a miR-122 backbone sequence, a miR-122_M backbone sequence, a miR-130a backbone sequence, a miR-16-2 backbone sequence, a miR-128 backbone sequence, a miR-144 backbone sequence, a miR-451a backbone sequence, or a miR-223 backbone sequence.

In some embodiments, the inhibitory nucleic acid is a miRNA comprising the nucleic acid sequence set forth in any one of SEQ ID NOS: 443-490, 1109-1111, 1114, 1121-1168, 1405-1520, 1908-2007, 2011, 2017, 2021, 2025, 2027, 2031, 2035, 2039, 2041, 2045, 2049, 2053, 2057, 2061, 2067, 2071, 2075, 2079, 2085, 2089, 2093, 2097, 2101, 2105, 2109, 2113, 2117, 2120, 2124, 2128, 2132, 2136, 2140, 2144, 2148, 2154, 2158, 2162, 2166, 2170, 2174, 2176, 2180, 2182, 2184, 2187, 2189, 2191, 2193, 2195, 2197, 2199, 2205, 2211, 2261, 2263, 2265, and 2267.

In some embodiments, the nucleic acid sequence encoding the inhibitory nucleic acid is located in an untranslated region of the expression construct. In some embodiments, the untranslated region is an intron, a 5′ untranslated region (5′UTR), or a 3′ untranslated region (3′UTR).

In some embodiments, the isolated nucleic acid comprising an expression construct encoding an inhibitory nucleic acid furthers comprises a promoter. In some embodiments, the promoter is a RNA pol III promoter (e.g., U6, H1, etc.), a chicken-beta actin (CBA) promoter, a CAG promoter, a H1 promoter, a CD68 promoter, a human synapsin promoter, or a JeT promoter. In some embodiments, the promoter is an H1 promoter comprising nucleotides 113-203 of SEQ ID NO:1522, nucleotides 1798-1888 of SEQ ID NO:1521, nucleotides 113-343 of SEQ ID NO:2257, or nucleotides 244-343 of SEQ ID NO:2257.

In some embodiments, the expression construct is flanked by a 5′ adeno-associated virus (AAV) inverted terminal repeat (ITR) sequence and a 3′ AAV ITR sequence, or variants thereof. In some embodiments, one of the ITR sequences lacks a functional terminal resolution site. In some embodiments, the ITRs are derived from an AAV serotype selected from the group consisting of: AAV1, AAV2, AAV5, AAV6, AAV6.2, AAV7, AAV8, AAV9, AAVRh10, AAV11, and variants thereof. In some embodiments, the 5′ ITR comprises nucleotides 1-106 of SEQ ID NO:2257 and the 3′ ITR comprises nucleotides 2192-2358 of SEQ ID NO:2257.

In another aspect, the disclosure provides a vector comprising the isolated nucleic acid as provided in the present disclosure. In some embodiments, the vector is a plasmid or viral vector. In some embodiments, the viral vector is a recombinant adeno-associated virus (rAAV) vector or a Baculovirus vector. In some embodiments, the vector is a self-complementary rAAV vector. In some embodiments, the vector (e.g., rAAV vector) further comprises a stuffer sequence. In some embodiments, the stuffer sequence comprises nucleotides 348-2228 of SEQ ID NO:1522 or nucleotides 489-2185 of SEQ ID NO:2257. In some embodiments, the vector (e.g., rAAV vector) comprises the nucleotide sequence of any one of SEQ ID NOS:2257-2260.

In another aspect, the disclosure provides a recombinant adeno-associated (rAAV) particle comprising the isolated nucleic acid molecule or rAAV vector as provided in the present disclosure. In some embodiments, the rAAV particle comprises a capsid protein. In some embodiments, the capsid protein is capable of crossing the blood-brain barrier. In some embodiments, the capsid protein is an AAV9 capsid protein or AAVrh.10 capsid protein. In some embodiments, the rAAV particle transduces neuronal cells and/or non-neuronal cells of the central nervous system (CNS).

In another aspect, the disclosure provides a pharmaceutical composition comprising the isolated nucleic acid as provided in the present disclosure, the vector as provided in the present disclosure, or the rAAV particle as provided in the present disclosure, and optionally a pharmaceutically acceptable carrier.

In another aspect, the disclosure provides a host cell comprising the isolated nucleic acid as provided in the present disclosure, the vector as provided in the present disclosure, or the rAAV particle as provided in the present disclosure.

In another aspect, the disclosure provides method for treating a subject having or suspected of having a neurodegenerative disease, the method comprising administering to the subject the isolated nucleic acid molecule as provided in the present disclosure, the vector as provided in the present disclosure, the rAAV particle as provided in the present disclosure, or the pharmaceutical composition as provided in the present disclosure. In some embodiments, the administration comprises direct injection to the CNS of the subject. In some embodiments, the direct injection is intracerebral injection, intraparenchymal injection, intrathecal injection, intrastriatal injection subpial injection, or any combination thereof. In some embodiments, the direct injection is direct injection to the cerebrospinal fluid (CSF) of the subject, optionally wherein the direct injection is intracistemal injection, intraventricular injection, and/or intralumbar injection. In some embodiments, the subject is characterized as having an ATXN2 allele having at least 22 CAG trinucleotide repeats, optionally wherein the ATXN2 allele has at least 24 CAG trinucleotide repeats, at least 27 CAG trinucleotide repeats, at least 30 CAG trinucleotide repeats, or at least 33 or more CAG trinucleotide repeats. In some embodiments, the neurodegenerative disease is spinocerebellar ataxia-2, amyotrophic lateral sclerosis, frontotemporal dementia, primary lateral sclerosis, progressive muscular atrophy, limbic-predominant age-related TDP-43 encephalopathy, chronic traumatic encephalopathy, dementia with Lewy bodies, corticobasal degeneration, progressive supranuclear palsy (PSP), dementia Parkinsonism ALS complex of guam (G-PDC), Pick's disease, hippocampal sclerosis, Huntington's disease, Parkinson's disease, or Alzheimer's disease.

In another aspect, the disclosure provides a method of inhibiting ATXN2 expression in a cell, the method comprising delivering to the cell the isolated nucleic acid molecule as provided in the present disclosure, the vector as provided in the present disclosure, the rAAV particle as provided in the present disclosure, or the pharmaceutical composition as provided in the present disclosure. In some embodiments, the cell has an ATXN2 allele having at least 22 CAG trinucleotide repeats, optionally wherein the ATXN2 allele has at least 24 CAG trinucleotide repeats, at least 27 CAG trinucleotide repeats, at least 30 CAG trinucleotide repeats, or at least 33 or more CAG trinucleotide repeats. In some embodiments, the cell is a cell in the CNS, optionally a neuron, glial cell, astrocyte, or microglial cell. In some embodiments, the cell is in vitro. In some embodiments, the cell is from a subject having one or more symptoms of a neurodegenerative disease. In some embodiments, the cell is from a subject having or suspected of having a neurodegenerative disease. In some embodiments, the neurodegenerative disease is spinocerebellar ataxia-2, amyotrophic lateral sclerosis, frontotemporal dementia, primary lateral sclerosis, progressive muscular atrophy, limbic-predominant age-related TDP-43 encephalopathy, chronic traumatic encephalopathy, dementia with Lewy bodies, corticobasal degeneration, progressive supranuclear palsy (PSP), dementia Parkinsonism ALS complex of guam (G-PDC), Pick's disease, hippocampal sclerosis, Huntington's disease, Parkinson's disease, or Alzheimer's disease.

In another aspect the present disclosure provides a method of inhibiting ATXN2 expression in the central nervous system of a subject, the method comprising administering to the subject the isolated nucleic acid molecule as provided in the present disclosure, the vector as provided in the present disclosure, the rAAV particle as provided in the present disclosure, or the pharmaceutical composition as provided in the present disclosure. In some embodiments, the administration comprises direct injection to the CNS of the subject. In some embodiments, the direct injection is intracerebral injection, intraparenchymal injection, intrathecal injection, intrastriatal injection, subpial injection, or any combination thereof. In some embodiments, the direct injection is injection to the cerebrospinal fluid (CSF) of the subject, optionally wherein the direct injection is intracistemal injection, intraventricular injection, and/or intralumbar injection. In some embodiments, the subject has an ATXN2 allele having at least 24 CAG trinucleotide repeats, at least 27 CAG trinucleotide repeats, at least 30 CAG trinucleotide repeats, or at least 33 or more CAG trinucleotide repeats.

In another aspect, the present disclosure provides an artificial miRNA comprising a guide strand sequence and a passenger strand sequence, wherein the guide strand sequence comprises any one of SEQ ID NOS: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 1176-1288, 1811-1827, 2015, 2065, 2083, 2152, 2203, and 2209.

In some embodiments, the guide strand sequence and passenger strand sequence are contained within a miR backbone sequence. In some embodiments, the miR backbone sequence is a miR-155 backbone sequence, a miR-155E backbone sequence, a miR-155M backbone sequence, miR1-1 backbone sequence, a miR-1-1_M backbone sequence, a miR-16-2 backbone sequence, a miR-100 backbone sequence, a miR-100_M backbone sequence, a miR-190a backbone sequence, a miR-190a_M backbone sequence, a miR-124 backbone sequence, a miR-124_M backbone sequence, a miR-132 backbone sequence, a miR-9 backbone sequence, a miR-138-2 backbone sequence, a miR-122 backbone sequence, a miR-122_M backbone sequence, a miR-130a backbone sequence, a miR-128 backbone sequence, a miR-144 backbone sequence, a miR-451a backbone sequence, or a miR-223 backbone sequence.

In some embodiments, the artificial miRNA comprises a sequence as set forth in any one of SEQ ID NOS: 443-490, 1109-1111, 1114, 1121-1168, 1405-1520, 1908-2007, 2011, 2017, 2021, 2025, 2027, 2031, 2035, 2039, 2041, 2045, 2049, 2053, 2057, 2061, 2067, 2071, 2075, 2079, 2085, 2089, 2093, 2097, 2101, 2105, 2109, 2113, 2117, 2120, 2124, 2128, 2132, 2136, 2140, 2144, 2148, 2154, 2158, 2162, 2166, 2170, 2174, 2176, 2180, 2182, 2184, 2187, 2189, 2191, 2193, 2195, 2197, 2199, 2205, 2211, 2261, 2263, 2265, and 2267.

In another aspect, the present disclosure provides an isolated RNA duplex comprising a guide strand sequence and a passenger strand sequence, wherein the guide strand sequence comprises the nucleic acid sequence set forth in any one of SEQ ID NOS: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, and 1176-1288, 1811-1827, 2015, 2065, 2083, 2152, 2203, and 2209, optionally wherein the guide strand sequence and passenger strand sequence are linked by a loop region to form a hairpin structure comprising a duplex structure and a loop region. In some embodiments, the loop structure comprises from 6 to 25 nucleotides.

In another aspect, the disclosure provides a kit comprising a container housing a composition as described by the present disclosure.

### BRIEF DESCRIPTION OF THE SEVERAL VIEWS OF THE DRAWINGS

FIG. 1 shows tuning mean squared error for mir-30 dataset (Pelossof et al., Nature Biotechnology (2017) 35:350-353). Data showing the mean squared error (MSE) for prediction performance of the shRNA prediction algorithm on a set of shRNAs targeting Kras, held out from a data set of shRNAs used to train the support vector machine model to predict shRNA performance. Mean squared error is calculated as the square of the difference between the score of the support vector machine (SVM) predictor and the label 1 or −1, corresponding to shRNAs empirically determined to yield good knockdown or poor knockdown. These squared differences are averaged across shRNAs tested. The hyperparameter c was varied and the mean squared errors calculated for each value c.

FIG. 2 shows a plot of precision vs recall for SVM model applied to held-out shRNAs targeting Trp53 gene, after training on the shRNAs targeting the other genes in the TILE dataset (Pelossof et al, Nature Biotechnology (2017) 35:350-353). Horizontal line at approximately 0.19 represents the fraction of shRNAs that are positive, i.e., yielding good knockdown, out of the total number of shRNAs, in the set of all shRNAs targeting Trp53. The precision-recall line represents, varying across values of the SVM score, the fraction of true positives that are included in the dataset (‘recall’), versus the fraction of true positives relative to false positives (‘precision’), at a given SVM score cutoff. Thus, at the least stringent SVM score, all true positives are included (recall=1), but precision is low because many negative shRNAs are included.

FIG. 3 shows two curves are plotted against SVM score. In one, the cumulative fraction of positive shRNAs that are expected to be lost as the classifier score is increased is shown. This is denoted by the bold line. In the other, the percent improvement in rejection of low-performing shRNAs is shown. This is denoted by the lighter line. Vertical dashed lines, from left to right, represent the 25^{th} percentile (light dashed) and 50^{th} percentile (bold dashed) of SVM scores in the dataset, the shRNAs targeting Trp53.

FIG. 4 shows jitter plots of the distribution of SVM score predictions as a function of the first base of the guide sequence of the shRNA sequences targeting ATXN2. All data points are shown; the horizontal width of the ‘violin’ is proportional to the number of points at each SVM score, which is plotted on the y axis. On the left, the score is calculated for guide sequences that are perfectly complementary to the ATXN2 sequence (guide sequence base at position 1 is A, U, C, or G). On the right, the score is calculated if the first base is converted to U (edit guide sequence base at position 1 to U if guide at position 1 does not natively begin with U). Note that guide sequences which originally begin with U will have the same score in the right plot, whereas sequences which begin with A, G, or C will have different scores. In general, the SVM score increases if the first base is U.

FIG. 5 shows a plot of ATXN2 quantigene assay values across a panel of commonly used cell lines. Signal is reported with 30 μL (left bar) or 10 μL (right bar) of lysate. “−” represents negative control with no cellular material. Y-axis is the assay signal. Additional horizontal line represents the minimal signal selection criterion.

FIGS. 6A-6B show a ‘Sashimi’ plot of the alternative splicing of Ataxin-2 transcript from human brain or from HepG2.5 cells. FIG. 6A: For brain, representative plots from two different individuals are shown. The height of the bars in the plot represents the number of reads aligning to the position in Ataxin-2, according to the diagram underneath the plot. Numbers on curved arcs represent the count of reads aligning across exon-exon junctions. Injunctions where the arcs are on both top and bottom, this indicates potential alternative splicing of the transcript. Arrows point out exons subject to substantial alternative splicing, such that these exons do not appear in a substantial number of ATXN2 transcripts in human brain. The diagram at the bottom of the plot represents the structure of the transcript ENST00000377617.7, with exons as solid rectangles. The transcript is oriented from right to left, with exon 1 on the right. FIG. 6B: Similar data from HepG2 cells is shown. The alignment to the transcript is not to scale.

FIG. 7 shows ATXN2 mRNA values across tested siRNAs, at 20 nM, 1 nM, and 200 pM doses. The x-axis shows the position of the ATXN2 sequence (SEQ ID NO: 2) that the corresponding siRNA is complementary to. ATXN2 mRNA values represents the ratio of ATXN2 to GAPDH signal from quantigene assay, normalized to mock control. 3′ UTR on the X-axis shows the general position of the 3′ untranslated region of the ATXN2 transcript.

FIG. 8. Correlation plot of the ATXN2 mRNA knockdown (ratio of ATXN2 to GAPDH signal, normalized to mock transfected controls), versus the SVM score. The expected correlation is observed, indicating that high SVM scores predict good knockdown performance.

FIG. 9. Plot of ATXN2 signal from ATXN2 siRNA treated U2OS cells, derived from indirect immunofluorescence, for the indicated conditions. XD-ID Nos represent treatment with different siRNAs corresponding to Table 1, at the indicated dose (20 nM (top) or 1 nM (bottom)). Other treatments are indicated as follows: “no_primary_secondary”=during antibody staining, the Ataxin-2 primary antibody was omitted, secondary antibody was included; “no_primary_no secondary”=during antibody staining, both the Ataxin-2 primary antibody and the secondary fluorescent antibody against the Ataxin-2 antibody were omitted; “primary_no secondary”=during antibody staining, the Ataxin-2 primary antibody was included but the secondary fluorescent antibody against the Ataxin-2 antibody was omitted; “SMP”=a pool of 4 siRNAs targeting ATXN2, with chemically modified nucleotides, obtained from Dharmacon; “primary_secondary”=untreated cells stained with primary and secondary antibody; “NTC”=cells treated with a ‘non-targeting control’ siRNA, not expected to target any human transcripts, with chemically modified nucleotides, obtained from Dharmacon; “XD-LucControl”=an siRNA, comprised only of RNA bases as in the ATXN2 targeting siRNAs, expected to target the luciferase gene but not to target ATXN2. In the plot, each point represents signal averaged across all cells in a well. Outliers, which were excluded from calculation of mean knockdown across wells in Tables 6 and 7, are shown as lighter colored points.

FIGS. 10A-10B show representative images of ATXN2 siRNA treated U2OS cells as described in FIG. 9. FIG. 10A: Representative images of siRNA (20 nM) treated U2OS cells. Top panels, Hoechst staining demarcates cell nuclei. Bottom panels, ATXN2 indirect immunofluorescence. Treatment/staining procedure is shown below image panels. FIG. 10B: As in FIG. 11A, but for U2OS samples treated with siRNAs at 1 nM.

FIG. 11 shows a plot of normalized ATXN2 indirect immunofluorescence signal, as a function of position along ATXN2 transcript (SEQ ID NO: 2). The x-axis is restricted to the positions along the ATXN2 transcript spanning the binding sites of the tested siRNAs.

FIGS. 12A-12C show dose response of various siRNAs tested. FIG. 12A (top) shows a plot of log IC50 across siRNA IDs tested in Group 1. Bars represent span of 95% confidence interval for IC50 values. FIG. 12A (bottom) shows representative dose response curves for siRNAs. Y-axis represents ratio of ATXN2 to GAPDH signal from quantigene assay of mRNA levels, from lysates of HepG2 cells dosed with indicated concentration of siRNA. Fits represents 3-parameter logistic regression fits, with Hill slope set constant at 1. Outliers were automatically identified, excluded from curve fitting and IC50 estimation. FIG. 12B shows a plot of log IC50 across siRNA IDs tested in Group 2. Bars represent span of 95% confidence interval for IC50 values. FIG. 12C shows a representative dose response curves for siRNAs. Y-axis represents ratio of ATXN2 to GAPDH signal from quantigene assay of mRNA levels, from lysates of HepG2 cells dosed with indicated concentration of siRNA. Fits represents 3-parameter logistic regression fits, with Hill slope set constant at 1. Outliers were automatically identified, excluded from curve fitting and IC50 estimation, and are indicated on graph.

FIG. 13 shows predicted folding patterns of guide sequences embedded in miRNA backbones, as created using the web-based server mfold. Multiple fold predictions are obtained; a representative fold is shown. Note the unpaired, ‘bulged’ nucleotides at several positions in each miRNA in the vicinity of the guide sequence, except in the ‘sealed’ variant.

FIG. 14 shows fluorescence automated cell sorting data demonstrating reduction in signal intensity for a GFP (stop)-ATXN2 reporter construct-expressing U2OS cell line by artificial miRNAs. Cells were transfected with vectors containing inserts either including the guide sequence of XD-14792 (SEQ ID NO:112), or control guide sequences, embedded in miRNA backbones. Y-axis plots the median fluorescence intensity of cells within each replicate. Replicates derive from wells of a 96-well plate containing cells that were transfected with vectors. The cells were dissociated with trypsin prior to FACS analysis.

FIG. 15 shows thresholding procedure to distinguish transduced from untransduced cells in imaging experiments using lentivirally packaged ATXN2-specific artificial miRNAs. Lentiviral vectors (similar to pLVX-EF1A_mCherry-miR-1-1-XD_14890-WPRE_CMV (SEQ ID NO:546)) express mCherry and so identification of mCherry expression distinguishes transduced from untransduced cells. Left panel shows histogram of signal in the fluorescence channel used to detect mCherry signal (including indirect immunofluorescence from an anti-mCherry antibody and fluorescent secondary antibody). Right panel shows histogram of signal from cells transduced with mCherry-encoding vector, with a clear bimodal distribution of signals representing untransduced cells (low signal) and transduced cells (high signal). Vertical line shows threshold used to separate mCherry positive from mCherry negative cells, placed such that no untransduced cells exceed this signal threshold and such that the large majority of the right peak of the bimodal histogram of mCherry signal in transduced cells exceeds this threshold.

FIG. 16 shows ATXN2 signal normalization procedure for artificial miRNA high content imaging assay. Each point represents signal in the channel used to detect indirect immunofluorescence for ATXN2, average across cells in the well. ATXN2 knockout cells were used to determine the background levels of indirect immunofluorescence for the ATXN2 antibody. The different cell types and staining conditions are shown, with the y-axis normalized with 100% set to the signal from wild-type, untransduced cells and 0% set to the signal from untransduced ATXN2 knockout cells. The signal in the ATXN2-antibody stained ATXN2 knockout cells somewhat exceeds signal from cells not stained with antibody, indicating that there is some background associated with the antibody and that using the ATXN2 knockout can help correct for this background to improve accuracy in measuring ATXN2 protein signal.

FIGS. 17A-17B show plots of ATXN2 signal from wells transduced with lentiviral vectors expressing guide sequences (shown on x-axis) embedded in miRNA backbones (miR-155E—FIG. 17A; miR1-1—FIG. 17B). Guide sequences and miRNA context sequences are listed in Table 11.

FIG. 18A-18B show representative images of Hoechst 33342 stain (top row), mCherry signal (middle row), and ATXN2 indirect immunofluorescence signal (bottom row) from cells as quantified in FIG. 17. FIG. 18A shows data for guide sequences embedded in miR-155E backbone; FIG. 18B shows data for guide sequences embedded in miR1-1 backbone.

FIG. 19 shows a plot of ATXN2 protein signal from miRNA-embedded anti-ATXN2 guide sequences versus ATXN2 mRNA signal from anti-ATXN2 siRNA treatment. There is correlation between the mRNA and protein knockdown across conditions tested.

FIGS. 20A-20C show validation of CRISPR guide RNAs in disrupting Ataxin-2 gene and knocking out Ataxin-2 protein in U2OS cells. FIG. 20A shows western blot analysis of U2OS cells nucleofected with ATXN2-targeting CRISPR gRNAs, complexed with Cas9 protein. Treatments include no nucleofection controls, control guide RNAs targeting CD81 or expected to be non-targeting, and five unique ATXN2 targeting guides. Immunoblots against Ataxin-2 protein and alpha-tubulin loading control are shown. FIG. 20B shows representative histograms and FIG. 20C shows median fluorescence intensity within treated wells of Ataxin-2 indirect immunofluorescence signal for cells nucleofected with indicated treatments, as in FIG. 20A.

FIGS. 21A-21B show U2OS ATXN2 knockout clones generated for assay calibration. FIG. 21A shows ATXN2 U2OS knockout cell line generation scheme. FIG. 21B shows western blot analysis from clonal lines generated after nucleofection with indicated ATXN2 targeting gRNA. The lane containing protein from lysed material from the clone (clone 43) selected for use is indicated by the arrow.

FIG. 22 show knockdown of Ataxin-2 protein in vivo after AAV vectorized amiRNA delivery. AAV encoding miRNAs XD-14792 or XD-14887, embedded in the miR-1-1 backbone, or a control construct lacking a miRNA, was delivered intravenously to adult wild-type mice by tail vein injection. 15 days after injection, animals were euthanized and livers were harvested and snap-frozen. GFP fluorescence, resulting from vector encoded GFP, was detected in the liver upon blue light illumination. FIG. 22 (left): Liver lysate was immunoblotted for Ataxin-2, beta-actin, and GFP (not shown). Each lane is derived from a different animal. FIG. 22 (right): Ataxin-2 signal was normalized to beta-actin signal. All miRNA-dosed animals had lower Ataxin-2 signal than animals dosed with control AAV vector. Each point represents ratio of Atxn2 to Beta actin signal from an individual animal.

FIGS. 23A-23B show quality metrics of pooled library screen of Atxn2-targeting miRNAs (“Deep Screen 1”). FIG. 23A shows a scatter plot comparing ratios of high- and low-sorted samples in two replicates, showing tight correlation. FIG. 23B shows correlation matrix between all samples tested. Spearman correlation was calculated between guide sequence count vectors between all samples.

FIG. 24 shows ratio baseline subtraction procedure. Raw count ratios (log-base 2 transformed) are shown on x-axis, for top, ATXN2-targeting miRNAs, and bottom, scrambled miRNAs. For subsequent calculations, the median of the ratio for the scrambled miRNAs was subtracted.

FIG. 25 shows a plot of ATXN2 signal depletion versus cell depletion. Each point represents a library element, containing a miRNA targeting either the ATXN2 transcript; a scrambled sequence; or a sequence targeting an essential gene and expected to reduce cell proliferation and/or viability. The x-axis is the average across replicates of the ratio of sequence counts derived from cells in the high- and low-ATXN2 FACS gate populations. The y-axis is the average across replicates of the ratio of sequence counts derived from HeLa cells after initial transduction and after 16 days. Points falling toward the bottom of the axis represents elements that were depleted from the 16 day timepoint relative to the initial transduction timepoint.

FIG. 26 shows a plot of ATXN2 signal depletion versus position on ATXN2 transcript of complementarity of guide sequence. Points toward the bottom represent guide sequences with greater knockdown of ATXN2; points toward the top of the y-axis represent guide sequences with less knockdown of ATXN2.

FIG. 27 shows a similar plot as in FIG. 26, but zoomed-in on the 3′ end of the ATXN2 transcript. In black are sequences deemed part of ‘hotspots’ in the 3′ UTR of the ATXN2 transcript.

FIG. 28 shows the percent of reads, averaged across scrambled guide sequences, that match to a guide sequence excised from the pri-miRNA at the indicated position. The diagram above shows an example sequence, where the bold text to the left is miR backbone sequence and the regular text is the guide sequence. Arrows and numbers indicated cleavage position (for the tiled screened described here, in the miR 16-2 backbone, Drosha is the expected enzyme for this cleavage event). The seed sequence for a guide sequence cut at the expected position is shown. The position of this seed sequence will shift if the guide position is cut out of the pri-miRNA at a different position from the expected position.

FIG. 29 shows representative images used in assessing the production of motor neurons in the stem cell differentiation protocols. Upper left image shows overlay of indirect immunofluorescence signal from anti-HB9 and anti-Beta 3 tubulin (TUJ1) antibodies. Upper right shows overlay of signal from anti-ISLET1 and TUJ1 signal. Lower left shows overlay of HB9, ISLET1, and TUJ1 signal. Bottom right shows overlay of HB9, ISLET1, TUJ1, and nuclear DAPI stain. In the images, neuronal processes are clearly seen as labeled by TUJ1 antibody. Neuronal nuclei are labeled by the motor neuron markers HB9 and Islet1, with 25-35% of neurons labeled with HB9, 50-60% labeled by Islet1, and 70-80% of cells positive for TUJ1 signal.

FIG. 30A-30C show data from an experiment testing knockdown of ATXN2 mRNA and protein after transduction of ATXN2-targeting amiRNAs in lentiviral format in stem-cell derived motor neurons. FIG. 30A is a schematic of the cassette packaged in lentiviral vectors, with an H1 promoter driving the artificial miRNA, followed by a Pol III termination signal (6T). After this miR expression cassette, a CMV Pol II promoter drives expression of the fluorescent reporter GFP, and is followed by a WPRE element to stabilize the GFP transcript. FIG. 30B shows data from qPCR against ATXN2 mRNA. Each dot represents a biological replicate derived from a distinct tissue culture well of motor neurons. Data represent average signal calculated from change in qPCR threshold (CT) for ATXN2 versus either GUSB or B2M. Bars are mean of replicates, and error bars are standard deviation across replicates. ATXN2 signal is normalized to levels measured from motor neurons growing in wells not treated with vector. Data from wells treated with a control lentiviral vector with the multiple cloning site (MCS) in place of the amiRNA is shown as “MCS.” Two amiRNAs were tested, with amiRNAs targeting indicated position in ATXN2 transcript (1784 or 4402) indicated; amiRs were embedded in the miR16-2 backbone. The guide sequence targeting ATXN2 position 1784 is also referred to as XD-14792. Lentiviral vectors were dosed at two concentrations. The viral dose to achieve a multiplicity of infection (MOI) of 2.5 or 4.5 was calculated based on titration in U2OS cells (FACS analysis of GFP signal, calculating % cells positive for GFP). Using these values and the number of neurons plated per well, the corresponding dose of vector to achieve MOI of 2.5 or 4.5 in the motor neuron cultures (calculated based on the U2OS infectivity) was used. Observation of GFP fluorescence in cultures confirmed that transduction was near complete, as expected if the U2OS MOI was similar to the motor neuron MOI. FIG. 30C shows assessment of ATXN2 protein assessment from cultures treated the same as in FIG. 30B. The top panel shows the Western blot, with clear evidence of reduction in signal in lanes with protein from wells treated with amiRNAs targeting ATXN2 versus untreated wells or wells treated with the control MCS vector. Bottom panel quantifies ATXN2 immunoblot signal, with each point representing a biological replicate, the bars representing mean across replicates and the error bars standard deviation.

FIG. 31. Data is presented from an experiment performed similarly to that shown in FIG. 30. In this experiment, the MOI (as calculated by infectivity in U2OS cells) was 3.5. Knockdown in motor neurons treated with lentiviral vectors with miR 16-2 backbone-embedded amiRNAs targeting indicated ATXN2 transcript position is shown. Horizontal dashed line represents the threshold of 80% knockdown. In this experiment, it is apparent that the amiRNAs targeting the ATXN2 transcript in the 3′ UTR do not yield the same level of knockdown as amiRNAs targeting the ATXN2 coding sequence. Bars show mean knockdown, normalized to wells not treated with lentiviral vector; each point is a biological replicate (neurons from an individual well), and error bars are standard deviations across replicates. As above, MCS represents a lentiviral vector with a control multiple cloning site in place of a miR cassette.

FIG. 32. 2% agarose TAE gel demonstrating truncations in miR16-2 backbone-embedded amiRNAs packaged in AAV9. AAV genomic DNA was column purified and concentration quantified by Qubit fluorometer. Equal amounts of vector genome DNA, by Qubit measurement, were loaded into gel and subject to electrophoresis. Note that the gel image shown was spliced together for clarity. Leftmost lane is a DNA size ladder, with indicated DNA sizes in kilobases shown. From left to right, samples are (all DNA derived from purified AAV vector genomes): (1) H1 promoter driving miR1-1 XD-14792 (1784), followed by CBh promoter driving GFP; (2) H1 promoter followed by a non-miR multiple cloning site, followed by stuffer sequence “AMELY_V1”; (3-11) From left to right, AAV with amiRNAs targeting ATXN2 at positions 1784, 1479, 1755, 3330, 4402, 4405, 4406, 4409, and 4502. Each lane has an amiRNA targeting ATXN2, in the same vector genome format as lane 2 replacing the MCS with the indicated miR cassette, with miR16-2 backbone. Note in all of the material from AAV genomes with miR16-2 backbone miR cassettes the presence of both an upper band, running at the intended size, as well as a faster migrating lower band.

FIG. 33A-33B. Data from Deep Screen 2 showing replicate to replicate consistency (FIG. 33A) and performance across miR backbones (FIG. 33B). In FIG. 33A, each point represents the relative abundance of a library element, with position on the x-axis representing the log 2 fold change in abundance between the 10^{th} percentile ATXN2 sort and unsorted cells from the first screen replicate, and the y-axis the corresponding log 2 fold change from the second screen replicate. Points on the far right of the graph represent data where the denominator in the ratio of sequence counts for sorted and unsorted cells is 0, and hence undefined when log-transformed. There is good correspondence between the replicates for elements exhibiting substantial knockdown (log 2 fold change <−1), but for inactive controls (including essential gene targeting amiRNAs, 911 controls, and scramble controls), there is more variability from replicate to replicate in this screen compared to Deep Screen 1. As a result there is some deviation from screen replicate to replicate in the negative control medians. No baseline subtraction was done because of the agreement in log 2 fold change values for active amiRNAs. In FIG. 33B, boxplots represent the ATXN2 knockdown performance across amiRNAs embedded in various miR backbones. In each boxplot, the center line is the median, the upper and lower edges of the box represent the 75^{th} and 25^{th} percentiles, and the line extends beyond the box edges to either the maxima/minima or 1.5 times the interquartile range (difference between 25^{th} and 75^{th} percentiles), whichever is closer to the median. Overlaying points (very faint, transparent) represent the ATXN2 knockdown signal from individual miRNAs. The y-axis represents the mean log 2 fold-change between the abundance of sequencing reads of elements detected in the 10^{th} percentile of ATXN2 signal relative to the abundance of the guide in unsorted cells. In this screen, the theoretical maximum fold-change is 10-fold between the 10^{th} percentile sort and un-sorted cells.

FIG. 34. Depletion of essential-gene targeting amiRNAs in various miR backbones at a late timepoint T₁ (18 days after transduction) versus an early timepoint T₀ (1 day after transduction). The y-axis represents the log₂ fold change in abundance between the two timepoints, and was not baseline subtracted. A similar ranking between the ‘performance’ of each miR backbone in inducing guide depletion over time, when expressing essential gene-targeting amiRNAs as in this figure, versus performance of miR backbones in ATXN2 knockdown when expressing ATXN2-targeting amiRNAs, as in FIG. 33, can be seen.

FIG. 35. Agarose gel with purified AAV vector genomes with various miR backbones, with amiRNA targeting Atxn2 at position 4402 (first 10) embedded, or targeting position 1784 (last 2; 1784 guide sequence is same as XD-14792). Note that image is spliced for clarity (to place lane including DNA size ladder immediately adjacent to relevant lanes). Some lanes have bands that both migrate differently than others (miR122, miR1-1-4402, miR-1-1XD14792), this is likely due to differences in loading or dye binding and not true migration differences. More importantly, across miR backbones there are differences in the relative intensity of the second most intense band, migrating farther than the most intense upper band which is the presumed intended vector genome. AAV vector genomes with miR100 and miR128 backbones in particular have a less intense faster migrating band than others.

FIG. 36. Agarose gel with AAV vector genomes derived from pools of cis plasmids. Each pool includes elements generated by PCR amplification from an oligonucleotide pool containing a mixture of amiRs embedded in multiple miR backbones, and the PCR primers used do not distinguish between parent and “_M” form miR backbones. Thus, the pool labeled miR-1-1 will include amiRs in backbones miR-1-1 and miR-1-1_M; the pool labeled miR-100 will contain miR-100 and miR-100_M backbones; the pool labeled miR-190a will contain miR-190a and miR-190a_M backbones; the pool miR-124 will contain miR-124 and miR-124_M backbones; the pool miR-138-2 will contain miR-138-2 and miR-138-2_M backbones. miR-155M and miR-155E, though not related to each other by the “_M” modification rules, also have high sequence similarity and therefore the pool labeled “miR-155M” likely contains a mix of miR-155M and miR-155E backbones. Each lane contains purified vector genome DNA from AAV generated with indicated plasmid pool. The last lane is derived from a mixture of the 5 micropools shown in the gel as well as micropools with miR backbones miR-124, miR-128, miR-138-2, miR-144, and miR-155M. As in FIG. 35, the AAV pool with the miR-100 backbone (dashed box) has a less intense faster migrating band than the other AAV pools.

FIG. 37. Data from Deep Screen 2, only including elements with miR-100 or miR-100_M backbones. As in FIG. 33A, each point represents the relative abundance of a library element, with position on the x-axis representing the log 2 fold change in abundance between the 10^{th} percentile ATXN2 sort and unsorted cells from the first screen replicate, and the y-axis the corresponding log 2 fold change from the second screen replicate.

FIG. 38. RT-ddPCR data demonstrating knockdown of ATXN2 mRNA in stem-cell derived motor neurons 7 days after treatment with scAAV-DJ vectors expressing ATXN2-targeting amiRNAs. Each point represents a biological replicate (a well of neurons treated with AAV at indicated dose of vector genomes per cell). Indicated amiRNAs, denoted as miR backbone—Atxn2 targeting position, mark x-axis. The amiRNAs were embedded in a self-complementary vector genome, with an H1 promoter driving the amiR, and a stuffer sequence modified from PSG11, “PSG11_V5” (nucleotides 489-2185 of SEQ ID NO:2257) 3′ of the miR cassette up to the wild-type ITR. The y-x is represents RT-ddPCR signal, with copies of each transcript per unit microliter derived from percentage of positive to negative droplets for primer/probesets specific to ATXN2, GUSB, or B2M. The points represent averages of ratios of ATXN2/GUSB and ATXN2/B2M ratios.

FIG. 39. This graph shows a RT-ddPCR experiment similar to that in FIG. 38, except spanning a broader range of indicated doses. Because of constraints on the number of available cells, not all amiRNAs were treated with all doses. In this experiment, the ATXN2 mRNA level is calculated by ATXN2/B2M RT-ddPCR ratios.

FIG. 40. Images of stem-cell derived motor neurons treated with scAAV-DJ vector as in FIGS. 38 and 39. Cells were treated with a dose of 1E4 vector genomes per cell. Representative images of DAPI stain (to label cell nuclei), indirect immunofluorescence signal for anti-ISL1 antibody (to label motor neurons), and TUJ1 signal, to label neuronal processes. No obvious differences were seen in neuronal processes between neurons treated with an active ATXN2-targeting amiRNA (1755) and an inactive (1755_911) amiRNA in scAAV-DJ. Panels at right (top) quantify total number of cells, defined by DAPI staining, and (bottom) quantify fraction of cells that are positive for ISL1. Each point represents average quantification across fields for a given well. Asterisks indicate significant (p<0.05) difference versus vehicle (PBS+0.001% PF-68) control, calculated by one-way ANOVA followed by Dunnett's multiple comparisons test. Vectors encode amiRNAs targeting indicated ATXN2 transcript position in miR100 or miR100_M backbone (FIGS. 38 and 39 show which amiRNA is in miR100 and which is in miR100_M backbones). “PBS” represents wells of motor neurons treated with vehicle (PBS+0.001% PF-68); GFP represents the amiRNA and GFP expressing vector H1-miR1-1.XD-14792-CBh-GFP packaged in scAAV-DJ.

FIGS. 41A-41C. Similar to FIG. 40, FIG. 41A shows representative images of neuronal morphology across stem-cell derived motor neuron treated with indicated scAAV-DJ vector encoding specified amiRNA, embedded in miR100 or miR100_M backbone vector. There is no readily apparent alteration in neuronal morphology for any treatment compared to vehicle. Total number of Hoechst+ nuclei (FIG. 41B) and the % of total nuclei that are Isl1+ (FIG. 41C) in AAV treated stem-cell derived motor neurons was quantified.

FIG. 42. Shows ‘volcano plots’ of RNAseq data, comparing gene expression in neurons treated with active amiRNA versus their inactive, ‘9-11’ control counterparts. The 911 controls do not reduce ATXN2 levels, but differ only by 3 nucleotides (bases 9, 10 and 11) from the active amiRNAs. Off-target effects of the amiRNAs not involving bases 9, 10 and 11 may therefore be conserved with the cognate non-911 control amiRNA, and the comparison can be considered to enrich the ‘on-target’ transcriptional impact of lowering Atxn2 levels. By far the most robust transcriptional effect observed in comparisons of miR100_1755 and miR100_2945 versus their 911 controls is ATXN2. In the plots, each point represents a gene (counts for different transcripts are collapsed gene-wise); the y-axis represents the nominal p value; the x-axis the log₂ fold change for gene expression between conditions. Data is derived from n=5-6 biological replicates per treatment. Neurons were treated with a dose of 1E4 vector genomes/cell, and RNA collected for RNAseq (quantseq) 7 days later.

FIG. 43. Panel of ‘volcano plots’ comparing each indicated amiR AAV treatment, with the same treatment conditions described as in FIG. 42, to all other amiRNA treatments shown (n=6 replicates/condition). Axes are as in FIG. 42; horizontal dashed line represents the false discovery rate threshold of 10%. Here, what are plotted are predicted off-target transcripts (with detectable expression levels in this system) for each amiR, that is transcripts with complementarity to bases 2-18 of the guide sequence with 2 or fewer mismatches. For most of the amiRNAs, none or only very few of the predicted off-targets are downregulated relative to the set of other amiRNAs, and exceed the 10% false discovery rate threshold.

FIG. 44. Plot of Atxn2 mRNA versus biodistribution of ATXN2 amiRNA expressing vectors (miR1-1-1784 (left) and miR100-3330 (right)) from mice dosed intrastriatally with vectors expressing indicated amiRNA AAV construct. Each point represents RT-ddPCR mRNA and vector distribution data from RNA and DNA isolated from an individual striatal biopsy, taking the average of Atxn2/Gusb and Atxn2/Tbp droplet ratios, normalized to vehicle treated animals. Multiple distinct vector formats are included, all with one version of the H1 promoter and various stuffer sequences.

FIGS. 45A-45B. Plot of Taqman qPCR data from striatal biopsies of animals dosed with indicated amiRNA AAV constructs (miR1784—FIG. 45A; miR3330—FIG. 45B). For each striatal biopsy assessed, two data points are shown: the y-axis plots the CT threshold difference between amplification of cDNA from an exogenous amiR and an endogenous miR, miR124; or the difference between amplification of two endogenous miRs. The x-axis shows the (log-base-2 transform of) vector distribution data, as in FIG. 44. Dashed lines are linear fits. Note that the relationship between CT and expression is of a form similar to expression ˜2\hat{ }^{CT}, consistent with the apparent linear relationship between CT difference and log₂ (vector genomes/diploid genome).

FIG. 46. qPCR data (a subset of the data shown in FIG. 45) is plotted against small RNAseq quantification of exogenous amiR expression/total miR expression, for RNA deriving from the same striatal punch biopsies. The relationship between the delta CT of exogenous amiR versus endogenous miR and small RNAseq quantification is separately fit to a linear model (linear regression) for each of the indicated amiRs. The slope of fits for the qPCR versus small RNAseq for the two amiRs are similar, and the fits are good as quantified by residuals, R².

FIG. 47. This graph shows use of the linear model in FIG. 46 to derive a predicted absolute amiR expression level, as a function of total miR expression, for the remaining samples that only had amiR expression measured by qPCR. This predicted amiR expression level is plotted on the x-axis. Each point represents an individual striatal punch biopsy. The y-axis represents the RT-ddPCR quantified Atxn2 mRNA level for that biopsy, same as in FIG. 44. A loess fit is used to separately fit a curve to data from biopsies from animals dosed with miR1-1.1784 expressing AAVs (black filled circles, dashed line); or miR100.3330 expressing AAVs (open diamonds; dotted line).

FIGS. 48A-48B. Liver enzyme data, alanine transaminase (ALT) (FIG. 48A) and aspartate aminotransferase (AST) (FIG. 48B) from blood collected from the submandibular vein, at 2 or 3 weeks after intravenous dosing of AAVs expressing indicated amiRs. Naïve animals were monitored in parallel.

FIG. 49. Plot of Atxn2 mRNA knockdown and vector distribution, as in FIG. 44, in striatal biopsies from animals dosed with AAVs expressing indicated amiRNAs. Lines represent loess (locally estimated scatterplot smoothing) fits for each series, implemented in R (stats::loess).

FIGS. 50A-50B. Expression of amiRNAs in tissue from animals dosed with AAVs expressing indicated amiRNAs. Liver tissue was analyzed from animals dosed intravenously (FIG. 50A); striatal tissue was analyzed from animals dosed via intrastriatal injection (FIG. 50B). amiRNA expression is plotted as normalized to total miRNA expression.

FIG. 51. Plot showing 5′ end homogeneity of processed miRNAs in striatal tissue in animals dosed intrastriatally. The y-axis (log₁₀ scale) plots cumulative sequencing reads, across all samples (n=4/AAV), for mature amiRNAs initiating at the ‘expected’ position 0, 5′ of the expected start site (negative numbers) or 3′ of the expected start site (positive numbers). For all of these amiRs, the vast majority of mature processed amiRNAs initiate at the expected start site.

FIGS. 52A-52D. (Top) Diagrams of representative predicted folding structures (mfold) of amiRNAs miR100_1755 (FIG. 52A), miR100_2586 (FIG. 52B), miR100_2945 (FIG. 52C), and miR100_3330 (FIG. 52D), embedded in miR100 backbone. Arrow indicates typical start position of processed miRNA guide strand. (Bottom) Observed small RNAseq sequencing reads. On the left are observed sequences, on the right the number of observations across all samples (n=3-4 liver, n=6 striatal biopsy). Note that the sequence reads are DNA, and in the corresponding miRNA the sequence would be generated by substituting “U” bases for “T” in the reads. A small number of sequences were fusions between the amiR and endogenous miRs, but these are considered to be artifacts of the ligation reaction during the small RNAseq procedure and were excluded. By comparing the observed sequences to the pri-miRNA sequence on top, it. An be noted that in some cases 3′ modifications are occurring, such as addition of ‘A’ or ‘U’ bases (‘T’ in the DNA sequencing reads) at the 3′ terminus of the amiRNA.

FIGS. 53A-53C show knockdown of Ataxin-2 protein in vivo after AAV9 vectorized miRNA delivery into cerebrospinal fluid. As in FIG. 22, AAVs encoding miRNAs XD-14792 or XD-14887, embedded in the miR-1-1 backbone, or a control construct lacking a miRNA, were dosed, in this case injected bilaterally intracerebroventricularly (ICV) in postnatal day 0 mice, 3 microliters per hemisphere. amiRNAs were expressed either under the control of the neuron-specific Synapsin promoter (as in nucleotides 1128-1575 of SEQ ID NO:622 or nucleotides 1128-1575 of SEQ ID NO:623), or the ubiquitous CAG promoter. Brain tissue (cortex) was harvested at indicated timepoint after injection. (FIG. 53A) Diagrams are shown of the expression cassettes used. (FIG. 53B) Representative immunoblot from Western analysis, similar to FIG. 22. Immunoblotting was performed against Ataxin-2, Beta-actin and GFP. For each treatment dose administered per hemisphere is listed, calculated by qPCR titering against the GFP region in the vector genome. In FIG. 53C, quantification of signal intensity of Atxn2 protein or GFP protein, normalized to total protein signal intensity (Revert 700, Licor), are shown. Atxn2 signal is scaled to the average of CAG-MCS and SYN-MCS controls at the indicated times, and GFP signal is scaled to the GFP maximum for the 4 week timepoint or to the average GFP signal of multiple CAG-MCS vector IV dosed liver samples that were loaded onto each Western blot for the 8 week timepoint. Each point represents data from an individual cortex (from a single animal), averaging across technical replicates. Error bars show standard deviation across technical replicates. A reduction in Atxn2 levels relative to control AAV vectors (MCS) is apparent for CAG vectors expressing the XD-14792 miR at 4 and 8 week timepoints, and for the 8 week timepoint for vectors with the Synapsin promoter.

FIGS. 54A-54B show representative immunofluorescence micrographs of tissue sections of cortex and cerebellum from animals dosed i.c.v. with AAV9 control or amiRNA vectors expressing (XD-14792 in miR-1-1 backbone, SEQ ID NO:1133), as in FIG. 53. Red corresponds to indirect immunofluorescence signal for anti-Atxn2 antibodies; Green to anti-GFP signal; and blue are nuclei (Dapi stained). In FIG. 54A, presumptive layer 5 cortical pyramidal neurons are seen, with apical dendrites projecting up in the image. Intensity from the GFP reporter is present in neurons, which are likely transduced with the AAV. On the left, GFP-expressing neurons in the animal transduced with the control amiRNA also have strong Atxn2 (red) signal, and neurons can be clearly seen with both GFP and Atxn2 signal. On the right, which corresponds to an image of tissue from an animal dosed with an ATXN2 amiRNA (XD-14792 in miR-1-1 backbone, SEQ ID NO:1133) expressing vector, by contrast, neurons with strong GFP intensity do not also have strong Atxn2 intensity, and overall the number of neurons with strong Atxn2 signal appears to be reduced. FIG. 54B shows similar results as FIG. 54A, but captures Purkinje cells in the cerebellum. On the right, the image shows Cerebellar tissue from an animal injected with Atxn2 amiRNA (XD-14792 in miR-1-1 backbone, SEQ ID NO:1133) expressing vector. GFP labeled, AAV transduced Purkinje cells do not have strong Atxn2 signal, whereas Purkinje cells lacking GFP transduction have strong Atxn2 expression. By contrast, on the left, which corresponds to an image from an animal dosed with control vector, cells with GFP signal also have Atxn2 signal.

### DETAILED DESCRIPTION

Expansions of ATXN2 polyglutamine repeat to a length of 34 or longer causes spinocerebellar ataxia type 2 (SCA2). Moreover, intermediate length polyglutamine expansions in ATXN2 increase risk of ALS. Reduction of ATXN2 levels has been demonstrated to have therapeutic benefit in animal models of spinocerebellar ataxia-2 and ALS. Knocking down the ATXN2 protein using nucleic acid based therapies alleviates the progressive neurodegeneration that occurs in animal models expressing a variant of the human ATXN2 containing an expanded polyglutamine repeat. In an animal model of ALS, which overexpresses the TDP-43 protein, a component of the most common neuropathology found in patients with ALS, animals normally develop a progressive death of motor neurons. However, breeding these animals with ATXN2 knock out mice dramatically increased survival time (Elden et al., Nature (2010) 466:7310). Similarly, reducing ATXN2 protein levels by introducing antisense oligonucleotide nucleic acids also increased survival of TDP-43 transgenic mice. Lowering ATXN2 levels markedly increased lifespan and improved motor function in TDP-43 transgenic mice and decreased the burden of TDP-43 inclusions. AXTN2 may modulate toxicity by affecting the aggregation propensity of TDP-43. TDP-43 proteinopathy has also been observed in a number of neurodegenerative diseases, including ALS, FTD, primary lateral sclerosis, progressive muscular atrophy, limbic-predominant age-related TDP-43 encephalopathy, chronic traumatic encephalopathy, dementia with Lewy bodies, corticobasal degeneration, progressive supranuclear palsy (PSP), dementia Parkinsonism ALS complex of guam (G-PDC), Pick's disease, hippocampal sclerosis, Huntington's disease, Parkinson's disease, and Alzheimer's disease. Thus, reducing ATXN2 levels may be useful for treating neurodegenerative diseases where ATXN2 is a causative agent (e.g., SCA2), as well as neurodegenerative diseases where ATXN2 is not the causative agent but modifies TDP-43 pathological aggregation.

Aspects of the invention relate to inhibitory nucleic acids (e.g., siRNAs, shRNAs, miRNAs, including artificial miRNAs) that when administered to a subject reduce the expression or activity of Ataxin-2 in the subject. Accordingly, compositions and methods provided in the present disclosure are useful for the treatment of neurodegenerative diseases, including spinocerebellar ataxia type 2 (SCA2), amyotrophic lateral sclerosis (ALS), Alzheimer's frontotemporal dementia (FTD), parkinsonism, and conditions associated with TDP-43 proteinopathies.

Prior to setting forth this disclosure in more detail, it may be helpful to an understanding thereof to provide definitions of certain terms to be used herein. Additional definitions are set forth throughout this disclosure.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the term “about” means±20% of the indicated range, value, or structure, unless otherwise indicated. It should be understood that the terms “a” and “an” as used herein refer to “one or more” of the enumerated components. The use of the alternative (e.g., “or”) should be understood to mean either one, both, or any combination thereof of the alternatives. As used herein, the terms “include,” “have” and “comprise” are used synonymously, which terms and variants thereof are intended to be construed as non-limiting.

As used herein, the term “nucleic acid” or “polynucleotide” refer to any nucleic acid polymer composed of covalently linked nucleotide subunits, such as polydeoxyribonucleotides or polyribonucleotides. Examples of nucleic acids include RNA and DNA.

As used herein, “RNA” refers to a molecule comprising one or more ribonucleotides and includes double-stranded RNA, single-stranded RNA, isolated RNA, synthetic RNA, recombinant RNA, as well as modified RNA that differs from naturally-occurring RNA by the addition, deletion, substitution, and/or alternation of one or more nucleotides. Nucleotides of RNA molecules may comprise standard nucleotides or non-standard nucleotides, such as non-naturally occurring nucleotides or chemically synthesized nucleotides.

As used herein, “DNA” refers to a molecule comprising one or more deoxyribonucleotides and includes double-stranded DNA, single-stranded DNA, isolated DNA, synthetic DNA, recombinant DNA, as well as modified DNA that differs from naturally-occurring DNA by the addition, deletion, substitution, and/or alteration of one or more nucleotides. Nucleotides of DNA molecules may comprise standard nucleotides or non-standard nucleotides, such as non-naturally occurring nucleotides or chemically synthesized nucleotides.

“Isolated” refers to a substance that has been isolated from its natural environment or artificially produced. As used herein with respect to a cell, “isolated” refers to a cell that has been isolated from its natural environment (e.g., from a subject, organ, tissue, or bodily fluid). As used herein with respect to a nucleic acid, “isolated” refers to a nucleic acid that has been isolated or purified from its natural environment (e.g., from a cell, cell organelle, or cytoplasm), recombinantly produced, amplified, or synthesized. In embodiments, an isolated nucleic acid includes a nucleic acid contained within a vector.

As used herein, the term “wild-type” or “non-mutant” form of a gene refers to a nucleic acid that encodes a protein associated with normal or non-pathogenic activity (e.g., a protein lacking a mutation, such as a repeat region expansion that results in higher risk of developing, onset, or progression of a neurodegenerative disease).

As used herein, the term “mutation” refers to any change in the structure of a gene, e.g., gene sequence, resulting in an altered form of the gene, which may be passed onto subsequent generations (hereditary mutation) or not (somatic mutation). Gene mutations include the substitution, insertion, or deletion of a single base in DNA or the substitution, insertion, deletion, or rearrangement of multiple bases or larger sections of genes or chromosomes, including repeat expansions.

As used herein, the term “Ataxin 2” or “ATNX2” refers to a protein encoded by the ATXN2 gene, which contains a polyglutamine (polyQ, CAG repeat) tract. ATXN2 gene or transcript may refer to normal alleles of ATXN2, which usually have 22 or 23 repeats, or mutated alleles having intermediate (˜24-32 repeats) or longer repeat expansions (˜33 to >100 repeats). In some embodiments, ATXN2 refers to mammalian ATNX2, including human ATXN2. In some embodiments, wild-type ATXN2 refers to a protein sequence of Q99700.2 as set forth in SEQ ID NO:1 or naturally occurring variants thereof. In some embodiments, wild-type ATXN2 nucleic acid refers to a nucleic acid sequence of NM_002973.3 (SEQ ID NO:2), ENST00000377617.7, ENST00000550104.5, ENST00000608853.5, or ENST00000616825.4, or naturally occurring variants thereof.

As used herein, the term “inhibitory nucleic acid” refers to a nucleic acid that comprises a guide strand sequence that hybridizes to at least a portion of a target nucleic acid, e.g., ATXN2 RNA, mRNA, pre-mRNA, or mature mRNA, and inhibits its expression or activity. An inhibitory nucleic acid may target a protein coding region (e.g., exon) or non-coding region (e.g., 5′UTR, 3′UTR, intron, etc.) of a target nucleic acid. In some embodiments, an inhibitory nucleic acid is a single stranded or double stranded molecule. An inhibitory nucleic acid may further comprise a passenger strand sequence on a separate strand (e.g., double stranded duplex) or in the same strand (e.g., single stranded, self-annealing duplex structure). In some embodiments, an inhibitory nucleic acid is an RNA molecule, such as a siRNA, shRNA, miRNA, or dsRNA.

As used herein, a “microRNA” or “miRNA” refers to a small non-coding RNA molecule capable of mediating silencing of a target gene by cleavage of the target mRNA, translational repression of the target mRNA, target mRNA degradation, or a combination thereof. Typically, miRNA is transcribed as a hairpin or stem-loop (e.g., having a self-complementary, single-stranded backbone) duplex structure, referred to as a primary miRNA (pri-miRNA), which is enzymatically processed (e.g., by Drosha, DGCR8, Pasha, etc.) into a pre-miRNA. Pre-miRNA is exported into the cytoplasm, where it is enzymatically processed by Dicer to produce a miRNA duplex with the passenger strand and then a single-stranded mature miRNA molecule, which is subsequently loaded into the RNA-induced silencing complex (RISC). Reference to a miRNA may include synthetic or artificial miRNAs.

As used herein, a “synthetic miRNA” or “artificial miRNA” or “amiRNA” refers to an endogenous, modified, or synthetic pri-miRNA or pre-miRNA (e.g., miRNA backbone or scaffold) in which the endogenous miRNA guide sequence and passenger sequence within the stem sequence have been replaced with a miRNA guide sequence and a miRNA passenger sequence that direct highly efficient RNA silencing of the targeted gene (see, e.g., Eamens et al. (2014), Methods Mol. Biol. 1062:211-224). In some embodiments, the nature of the complementarity of the guide and passenger sequences (e.g., number of bases, position of mismatches, types of bulges, etc.) can be similar or different from the nature of complementarity of the guide and passenger sequences in the endogenous miRNA backbone upon which the synthetic miRNA is constructed.

As used herein, the term “microRNA backbone,” “miR backbone,” “microRNA scaffold,” or “miR scaffold” refers to a pri-miRNA or pre-miRNA scaffold, with the stem sequence replaced by a miRNA of interest, and is capable of producing a functional, mature miRNA that directs RNA silencing at the gene targeted by the miRNA of interest. A miR backbone comprises a 5′ flanking region (also referred to 5′ miR context, ≥9 nucleotides), a stem region comprising the miRNA duplex (guide strand sequence and passenger strand sequence) and basal stem (5′ and 3′, each about 4-13 nucleotides), at least one loop motif region including the terminal loop (≥10 nucleotides for terminal loop), a 3′ flanking region (also referred to 3′ miR context, ≥9 nucleotides), and optionally one or more bulges in the stem. A miR backbone may be derived completely or partially from a wild type miRNA scaffold or be a completely artificial sequence.

As used herein, the term “antisense strand sequence” or “guide strand sequence” of an inhibitory nucleic acid refers to a sequence that is substantially complementary (e.g., at least 45%, 50%, 55%, 60%, 65%, 70%, 75%, 80%, 85%, 90%, 95%, or 100% complementary) to a region of about 10-50 nucleotides (e.g., about 15-30, 16-25, 18-23, or 19-22 nucleotides) of the mRNA of the gene targeted for silencing. The antisense sequence is sufficiently complementary to the target mRNA sequence to direct target-specific silencing, e.g., to trigger the destruction of the target mRNA by the RNAi machinery or process. In some embodiments, the antisense sequence or guide strand sequence refers to the mature sequence remaining following cleavage by Dicer.

As used herein, the term “sense sequence” or “passenger strand sequence” of an inhibitory nucleic acid refers to a sequence that is homologous to the target mRNA and partially or completely complementary to the antisense strand sequence or guide strand sequence of an inhibitory nucleic acid. The antisense strand sequence and sense strand sequence of an inhibitory nucleic acid are hybridized to form a duplex structure (e.g., forming a double-stranded duplex or single-stranded self-annealing duplex structure). In some embodiments, the sense sequence or passenger strand sequence refers to the mature sequence remaining following cleavage by Dicer.

As used herein, a “duplex,” when used in reference to an inhibitory nucleic acid, refers to two nucleic acid strands (e.g., a guide strand and passenger strand) hybridizing together to form a duplex structure. A duplex may be formed by two separate nucleic acid strands or by a single nucleic acid strand having a region of self-complementarity (e.g., hairpin or stem-loop).

As used herein, the term “complementary” refers to the ability of polynucleotides to form base pairs with each other. Base pairs are typically formed by hydrogen bonds between nucleotide subunits in antiparallel polynucleotide strands or a single, self-annealing polynucleotide strand. Complementary polynucleotide strands can form base pairs in the Watson-Crick manner (e.g., A to T, A to U, C to G), or in any other manner that allows for the formation of duplexes. As apparent to skilled persons in the art, when using RNA as opposed to DNA, uracil rather than thymine is the base that is considered to be complementary to adenosine. Furthermore, when a “U” is denoted in the context of the present invention, the ability to substitute a “T” is understood, unless otherwise stated. Complementarity also encompasses Watson-Crick base pairing between non-modified and modified nucleobases (e.g., 5-methyl cytosine substituted for cytosine). Full complementarity, perfect complementarity or 100% complementarity between two polynucleotide strands is where each nucleotide of one polynucleotide strand can form hydrogen bond with a nucleotide unit of a second polynucleotide strand. % complementarity refers to the number of nucleotides of a contiguous nucleotide sequence in a nucleic acid molecule that are complementary to an aligned reference sequence (e.g., a target mRNA, passenger strand), divided by the total number of nucleotides and multiplying by 100. In such an alignment, a nucleobase/nucleotide which does not form a base pair is called a mismatch. Insertions and deletions are not permitted in calculating % complementarity of a contiguous nucleotide sequence. It is understood by skilled persons in the art that in calculating complementarity, chemical modifications to nucleobases are not considered as long as the Watson-Crick base pairing capacity of the nucleobase is retained (e.g., 5-methyl cytosine is considered the same as cytosine for the purpose of calculating % complementarity).

The “percent identity” between two or more nucleic acid sequences refers to the proportion nucleotides of a contiguous nucleotide sequence in a nucleic acid molecule that are shared by a reference sequence (i.e., % identity=number of identical nucleotides/total number of nucleotides in the aligned region (e.g., the contiguous nucleotide sequence)×100). Insertions and deletions are not permitted in the calculation of % identity of a contiguous nucleotide sequence. It is understood by skilled persons in the art that in calculating identity, chemical modifications to nucleobases are not considered as long as the Watson-Crick base pairing capacity of the nucleobase is retained (e.g., 5-methyl cytosine is considered the same as cytosine for the purpose of calculating % identity).

As used herein, the term “hybridizing” or “hybridizes” refers to two nucleic acids strands forming hydrogen bonds between base pairs on antiparallel strands, thereby forming a duplex. The strength of hybridization between two nucleic acid strands may be described by the melting temperature (Tₘ), defined as at a given ionic strength and pH, the temperature at which 50% of a target sequence hybridizes to a complementary polynucleotide.

As used herein, “expression construct” refers to any type of genetic construct containing a nucleic acid (e.g., transgene) in which part or all of the nucleic acid encoding sequence is capable of being transcribed. In some embodiments, expression includes transcription of the nucleic acid, for example, to generate a biologically-active polypeptide product or inhibitory RNA (e.g., siRNA, shRNA, miRNA) from a transcribed gene. In some embodiments, the transgene is operably linked to expression control sequences.

As used herein, the term “transgene” refers to an exogenous nucleic acid that has been transferred naturally or by genetic engineering means into another cell and is capable of being transcribed, and optionally translated.

As used herein, the term “gene expression” refers to the process by which a nucleic acid is transcribed from a nucleic acid molecule, and often, translated into a peptide or protein. The process can include transcription, post-transcriptional control, post-transcriptional modification, translation, post-translational control, post translational modification, or any combination thereof. Reference to a measurement of “gene expression” may refer to measurement of the product of transcription (e.g., RNA or mRNA), the product of translation (e.g., peptides or proteins).

As used herein, the term “inhibit expression of a gene” means to reduce, down-regulate, suppress, block, lower, or stop expression of the gene. The expression product of a gene can be a RNA molecule transcribed from the gene (e.g., an mRNA) or a polypeptide translated from an mRNA transcribed from the gene. Typically a reduction in the level of an mRNA results in a reduction in the level of a polypeptide translated therefrom. The level of expression may be determined using standard techniques for measuring mRNA or protein.

As used herein, “vector” refers to a genetic construct that is capable of transporting a nucleic acid molecule (e.g., transgene encoding inhibitory nucleic acid) between cells and effecting expression of the nucleic acid molecule when operably-linked to suitable expression control sequences. Expression control sequences may include transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. The vector may be a plasmid, phage particle, transposon, cosmid, phagemid, chromosome, artificial chromosome, virus, virion, etc. Once transformed into a suitable host cell, the vector may replicate and function independently of the host genome, or may, in some instances, integrate into the genome itself.

As used herein, “host cell” refers to any cell that contains, or is capable of containing a composition of interest, e.g., an inhibitory nucleic acid. In embodiments, a host cell is a mammalian cell, such as a rodent cell, (mouse or rat) or primate cell (monkey, chimpanzee, or human). In embodiments, a host cell may be in vitro or in vivo. In embodiments, a host cell may be from an established cell line or primary cells. In embodiments, a host cell is a cell of the CNS, such as a neuron, glial cell, astrocyte, and microglial cell.

As used herein, “neurodegenerative disease” or “neurodegenerative disorder” refers to diseases or disorders that exhibit neural cell death as a pathological state. A neurodegenerative disease may exhibit chronic neurodegeneration, e.g., slow, progressive neural cell death over a period of several years, or acute neurodegeneration, e.g., sudden onset or neural cell death. Examples of chronic, neurodegenerative diseases include Alzheimer's disease, Parkinson's disease, Huntington's disease, spinocerebellar ataxia type 2 (SCA2), frontotemporal dementia (FTD), and amyotrophic lateral schlerosis (ALS). Chronic neurodegenerative diseases include diseases that feature TDP-43 proteinopathy, which is characterized by nucleus to cytoplasmic mislocalization, deposition of ubiquitinated and hyper-phosphorylated TDP-43 into inclusion bodies, protein truncation leading to formation of toxic C-terminal TDP-43 fragments, and protein aggregation. TDP-43 proteinopathy diseases include ALS, FTD, primary lateral sclerosis, progressive muscular atrophy, limbic-predominant age-related TDP-43 encephalopathy, chronic traumatic encephalopathy, dementia with Lewy bodies, corticobasal degeneration, progressive supranuclear palsy (PSP), dementia Parkinsonism ALS complex of guam (G-PDC), Pick's disease, hippocampal sclerosis, Huntington's disease, Parkinson's disease, and Alzheimer's disease. Acute neurodegeneration may be caused by ischemia (e.g., stroke, traumatic brain injury), axonal transection by demyelination or trauma (e.g., spinal cord injury or multiple sclerosis). A neurodegenerative disease may exhibit death of mainly one type of neuron or of multiple types of neurons.

As used herein, “subject,” “patient,” and “individual” are used interchangeably herein and refer to living organisms (e.g., mammals) selected for treatment or therapy. Examples of subjects include human and non-human mammals, such as primates (monkey, chimpanzee), cows, horses, sheep, dogs, cats, rats, mice, guinea pigs, pigs, and transgenic species thereof.

### Inhibitory Nucleic Acids

In one aspect, the disclosure provides isolated inhibitory nucleic acids that inhibit expression or activity of Ataxin 2 (ATXN2). The inhibitory nucleic acid is a nucleic acid that specifically binds (e.g., hybridizes to) at least a portion of the ATXN2 nucleic acid, such as an ATXN2 RNA, pre-mRNA, mRNA, and inhibits its expression or activity. In some embodiments, the inhibitory nucleic acid is complementary to a protein coding region or non-coding region (e.g., 5′UTR, 3′UTR, intron, etc.) of ATXN2. In some embodiments, the inhibitory nucleic acid is complementary to a wild type ATXN2 nucleic acid or a naturally occurring variant thereof. In some embodiments, the ATXN2 gene encodes a polypeptide identified by NCBI Reference Sequence NP_002964.4 or NP_002964.3. In some embodiments, an ATXN2 transcript comprises the sequence set forth in SEQ ID NO:2 or encodes an amino acid sequence set forth in SEQ ID NO:1. In some embodiments, the ATXN2 allele contains approximately 22 CAG trinucleotide repeats. In some embodiments, the ATXN2 allele has at least 22 CAG trinucleotide repeats, at least 24 CAG trinucleotide repeats, at least 27 CAG trinucleotide repeats, at least 30 CAG trinucleotide repeats, or at least 33 or more CAG trinucleotide repeats. In some embodiments, the inhibitory nucleic acid is single stranded or double-stranded. In some embodiments, the inhibitory nucleic acid is a siRNA, shRNA, miRNA, or dsRNA.

In some embodiments, the inhibitory nucleic acid is capable of inhibiting expression or activity of ATXN2 by at least 10%, at least 15%, at least 20%, at least 25%, at least 30%, at least 35%, at least 40%, at least 45%, at least 50%, at least 60%, at least 70%, at least 80%, at least 90% at least 95% or more in a cell compared to the expression level of ATXN2 in a cell that has not been contacted with the inhibitory nucleic acid. In some embodiments, the inhibitory nucleic acid is capable of inhibiting expression or activity of ATXN2 by 10-20%, 10-30%, 10-40%, 10-50%, 10-60%, 10-70%, 10-80%, 10-90%, 10-95%, 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% compared to the expression level of ATXN2 in a cell that has not been contacted with the inhibitory nucleic acid. Methods of measuring ATXN2 expression, e.g., levels of RNA, mRNA polypeptides, are known in the art including those described herein.

In some embodiments, the inhibitory nucleic acid comprises a guide strand sequence comprising or consisting of the nucleic acid sequence set forth in any one of the guide sequences in Tables 1, 3, 9, 11, 12, 13, 19, 23, 24, and 25. In some embodiments, the inhibitory nucleic acid comprises a guide strand sequence comprising or consisting of the nucleic acid sequence set forth in any one of SEQ ID NOS: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 1176-1288, 1811-1827, 2015, 2065, 2083, 2152, 2203, and 2209.

In some embodiments, the inhibitory nucleic acid comprises a guide strand sequence comprising or consisting of the nucleic acid sequence set forth in any one of the guide sequences in Tables 1, 3, 9, 11, 12, 13, 19, 23, 24, and 25, e.g., any one of SEQ ID NOS: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 1176-1288, 1811-1827, 2015, 2065, 2083, 2152, 2203, and 2209, with at least 1, 2, 3, 4, or 5 mismatches to the target ATXN2 mRNA sequence.

In some embodiments, the inhibitory nucleic acid comprises a guide strand sequence comprising or consisting of a nucleic acid sequence that is at least 60%, 70%, 75%, 80%, 85%, 90%, 95%, 97%, 99%, or 100% identical to any one of the guide sequences in Tables 1, 3, 9, 11, 12, 13, 19, 23, 24, and 25, e.g., any one of SEQ ID NOS: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 1176-1288, 1811-1827, 2015, 2065, 2083, 2152, 2203, and 2209.

In some embodiments, the inhibitory nucleic acid comprises a guide strand sequence comprising or consisting of at least 15, 16, 17, 18, 19, 20, 21, or 22 contiguous nucleotides of a sequence of any one of the guide sequences in Tables 1, 3, 9, 11, 12, 13, 19, 23, 24, and 25, e.g., any one of SEQ ID NOS: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 1176-1288, 1811-1827, 2015, 2065, 2083, 2152, 2203, and 2209, preferably wherein the guide strand sequence retains positions 2-7 (“seed sequence”) of the selected SEQ ID NO.

In some embodiments, the inhibitory nucleic acid comprises a guide strand sequence comprising or consisting of a sequence of any one of the guide sequences of Tables 1, 3, 9, 11, 12, 13, 19, 23, 24, and 25, e.g., any one of SEQ ID NOS: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 1176-1288, 1811-1827, 2015, 2065, 2083, 2152, 2203, and 2209, wherein 1, 2, 3, or 4 nucleotides at positions 19-22 differ from the selected SEQ ID NO (variant nucleotide(s)) such that the guide strand sequence is no longer complementary to the ATXN2 target sequence at the variant nucleotide(s).

In some embodiments, the inhibitory nucleic acid comprises a guide strand sequence of Table 12. In some embodiments, the inhibitory nucleic acid comprises a guide strand sequence comprising or consisting of the nucleic acid sequence set forth in any one of SEQ ID NOS: 12, 14, 40, 60, 100, 104, 108, 112, 124, 126, 128, 166, 198, 220, 242, 302, 306, 308, 330, 336, and 362. In some embodiments, the inhibitory nucleic acid comprises a guide strand sequence comprising or consisting of the nucleic acid sequence set forth in any one of SEQ ID NOS: 12, 14, 40, 60, 100, 104, 108, 112, 124, 126, 128, 166, 198, 220, 242, 302, 306, 308, 330, 336, and 362 with at least 1, 2, 3, 4, or 5 mismatches to the target ATXN2 mRNA sequence. In some embodiments, the inhibitory nucleic acid comprises a guide strand sequence comprising or consisting of a nucleic acid sequence that is at least 60%, 70%, 75%, 80%, 85%, 90%, 95%, 97%, 99%, or 100% identical to any one of SEQ ID NOS: 12, 14, 40, 60, 100, 104, 108, 112, 124, 126, 128, 166, 198, 220, 242, 302, 306, 308, 330, 336, and 362. In some embodiments, the inhibitory nucleic acid comprises a guide strand sequence comprising or consisting of at least 15, 16, 17, 18, 19, 20, 21, or 22 contiguous nucleotides of a sequence of any one of SEQ ID NOS: 12, 14, 40, 60, 100, 104, 108, 112, 124, 126, 128, 166, 198, 220, 242, 302, 306, 308, 330, 336, and 362, preferably wherein the guide strand sequence retains positions 2-7 (“seed sequence”) of the selected SEQ ID NO. In some embodiments, the inhibitory nucleic acid comprises a guide strand sequence comprising or consisting of a sequence of any one of SEQ ID NOS: 12, 14, 40, 60, 100, 104, 108, 112, 124, 126, 128, 166, 198, 220, 242, 302, 306, 308, 330, 336, and 362, wherein 1, 2, 3, or 4 nucleotides at positions 19-22 differ from the selected SEQ ID NO (variant nucleotide(s)) such that the guide strand sequence is no longer complementary to the ATXN2 target sequence at the variant nucleotide(s).

In some embodiments, the inhibitory nucleic acid comprises a guide strand sequence of Table 13. In some embodiments, the inhibitory nucleic acid comprises a guide strand sequence comprising or consisting of the nucleic acid sequence set forth in any one of SEQ ID NOS: 14, 40, 100, 108, 112, 128, 166, 198, 242, 308, 336, and 362. In some embodiments, the inhibitory nucleic acid comprises a guide strand sequence comprising or consisting of the nucleic acid sequence set forth in any one of SEQ ID NOS: 14, 40, 100, 108, 112, 128, 166, 198, 242, 308, 336, and 362 with at least 1, 2, 3, 4, or 5 mismatches to the target ATXN2 mRNA sequence. In some embodiments, the inhibitory nucleic acid comprises a guide strand sequence comprising or consisting of a nucleic acid sequence that is at least 60%, 70%, 75%, 80%, 85%, 90%, 95%, 97%, 99%, or 100% identical to any one of SEQ ID NOS: 14, 40, 100, 108, 112, 128, 166, 198, 242, 308, 336, and 362. In some embodiments, the inhibitory nucleic acid comprises a guide strand sequence comprising or consisting of at least 15, 16, 17, 18, 19, 20, 21, or 22 contiguous nucleotides of a sequence of any one of SEQ ID NOS: 14, 40, 100, 108, 112, 128, 166, 198, 242, 308, 336, and 362, preferably wherein the guide strand sequence retains positions 2-7 (“seed sequence”) of the selected SEQ ID NO. In some embodiments, the inhibitory nucleic acid comprises a guide strand sequence comprising or consisting of a sequence of any one of SEQ ID NOS: 14, 40, 100, 108, 112, 128, 166, 198, 242, 308, 336, and 362, wherein 1, 2, 3, or 4 nucleotides at positions 19-22 differ from the selected SEQ ID NO (variant nucleotide(s)) such that the guide strand sequence is no longer complementary to the ATXN2 target sequence at the variant nucleotide(s).

In some embodiments, the inhibitory nucleic acid comprises a guide strand sequence of Table 19. In some embodiments, the inhibitory nucleic acid comprises a guide strand sequence comprising or consisting of the nucleic acid sequence set forth in any one of SEQ ID NOS:1176-1288, 40, 108, and 166. In some embodiments, the inhibitory nucleic acid comprises a guide strand sequence comprising or consisting of the nucleic acid sequence set forth in any one of SEQ ID NOS:1176-1288, 40, 108, and 166, with at least 1, 2, 3, 4, or 5 mismatches to the target ATXN2 mRNA sequence. In some embodiments, the inhibitory nucleic acid comprises a guide strand sequence comprising or consisting of a nucleic acid sequence that is at least 60%, 70%, 75%, 80%, 85%, 90%, 95%, 97%, 99%, or 100% identical to any one of SEQ ID NOS:1176-1288, 40, 108, and 166. In some embodiments, the inhibitory nucleic acid comprises a guide strand sequence comprising or consisting of at least 15, 16, 17, 18, 19, 20, 21, or 22 contiguous nucleotides of a sequence of any one of SEQ ID NOS: 1176-1288, 40, 108, and 166, preferably wherein the guide strand sequence retains positions 2-7 (“seed sequence”) of the selected SEQ ID NO. In some embodiments, the inhibitory nucleic acid comprises a guide strand sequence comprising or consisting of a sequence of any one of SEQ ID NOS:1176-1288, 40, 108, and 166, wherein 1, 2, 3, or 4 nucleotides at positions 19-22 differ from the selected SEQ ID NO (variant nucleotide(s)) such that the guide strand sequence is no longer complementary to the ATXN2 target sequence at the variant nucleotide(s).

In some embodiments, the inhibitory nucleic acid comprises a guide strand sequence of Table 23. In some embodiments, the inhibitory nucleic acid comprises a guide strand sequence comprising or consisting of the nucleic acid sequence set forth in any one of SEQ ID NOS:1908-2007. In some embodiments, the inhibitory nucleic acid comprises a guide strand sequence comprising or consisting of the nucleic acid sequence set forth in any one of SEQ ID NOS:1908-2007, with at least 1, 2, 3, 4, or 5 mismatches to the target ATXN2 mRNA sequence. In some embodiments, the inhibitory nucleic acid comprises a guide strand sequence comprising or consisting of a nucleic acid sequence that is at least 60%, 70%, 75%, 80%, 85%, 90%, 95%, 97%, 99%, or 100% identical to any one of SEQ ID NOS:1908-2007. In some embodiments, the inhibitory nucleic acid comprises a guide strand sequence comprising or consisting of at least 15, 16, 17, 18, 19, 20, 21, or 22 contiguous nucleotides of a sequence of any one of SEQ ID NOS:1908-2007, preferably wherein the guide strand sequence retains positions 2-7 (“seed sequence”) of the selected SEQ ID NO. In some embodiments, the inhibitory nucleic acid comprises a guide strand sequence comprising or consisting of a sequence of any one of SEQ ID NOS:1908-2007, wherein 1, 2, 3, or 4 nucleotides at positions 19-22 differ from the selected SEQ ID NO (variant nucleotide(s)) such that the guide strand sequence is no longer complementary to the ATXN2 target sequence at the variant nucleotide(s).

In some embodiments, the inhibitory nucleic acid comprises a guide strand sequence of Table 24. In some embodiments, the inhibitory nucleic acid comprises a guide strand sequence comprising or consisting of the nucleic acid sequence set forth in any one of SEQ ID NOS:100, 112, 166, 202, 246, 306, 308, 314, 1180, 1185, 1196, 1200, 1211, 1213, 1215, 1216, 1224, 1811-1822, 1824-1827, 2015, 2065, 2083, 2152, 2203, and 2209. In some embodiments, the inhibitory nucleic acid comprises a guide strand sequence comprising or consisting of the nucleic acid sequence set forth in any one of SEQ ID NOS:100, 112, 166, 202, 246, 306, 308, 314, 1180, 1185, 1196, 1200, 1211, 1213, 1215, 1216, 1224, 1811-1822, 1824-1827, 2015, 2065, 2083, 2152, 2203, and 2209, with at least 1, 2, 3, 4, or 5 mismatches to the target ATXN2 mRNA sequence. In some embodiments, the inhibitory nucleic acid comprises a guide strand sequence comprising or consisting of a nucleic acid sequence that is at least 60%, 70%, 75%, 80%, 85%, 90%, 95%, 97%, 99%, or 100% identical to any one of SEQ ID NOS:100, 112, 166, 202, 246, 306, 308, 314, 1180, 1185, 1196, 1200, 1211, 1213, 1215, 1216, 1224, 1811-1822, 1824-1827, 2015, 2065, 2083, 2152, 2203, and 2209. In some embodiments, the inhibitory nucleic acid comprises a guide strand sequence comprising or consisting of at least 15, 16, 17, 18, 19, 20, 21, or 22 contiguous nucleotides of a sequence of any one of SEQ ID NOS:100, 112, 166, 202, 246, 306, 308, 314, 1180, 1185, 1196, 1200, 1211, 1213, 1215, 1216, 1224, 1811-1822, 1824-1827, 2015, 2065, 2083, 2152, 2203, and 2209, preferably wherein the guide strand sequence retains positions 2-7 (“seed sequence”) of the selected SEQ ID NO. In some embodiments, the inhibitory nucleic acid comprises a guide strand sequence comprising or consisting of a sequence of any one of SEQ ID NOS:100, 112, 166, 202, 246, 306, 308, 314, 1180, 1185, 1196, 1200, 1211, 1213, 1215, 1216, 1224, 1811-1822, 1824-1827, 2015, 2065, 2083, 2152, 2203, and 2209, wherein 1, 2, 3, or 4 nucleotides at positions 19-22 differ from the selected SEQ ID NO (variant nucleotide(s)) such that the guide strand sequence is no longer complementary to the ATXN2 target sequence at the variant nucleotide(s).

In some embodiments, the inhibitory nucleic acid comprises a guide strand sequence of Table 25. In some embodiments, the inhibitory nucleic acid comprises a guide strand sequence comprising or consisting of the nucleic acid sequence set forth in any one of SEQ ID NOS:1185, 1816, 1213, 1819, 2083, 1215, 1216, 1811, and 314. In some embodiments, the inhibitory nucleic acid comprises a guide strand sequence comprising or consisting of the nucleic acid sequence set forth in any one of SEQ ID NOS:1185, 1816, 1213, 1819, 2083, 1215, 1216, 1811, and 314, with at least 1, 2, 3, 4, or 5 mismatches to the target ATXN2 mRNA sequence. In some embodiments, the inhibitory nucleic acid comprises a guide strand sequence comprising or consisting of a nucleic acid sequence that is at least 60%, 70%, 75%, 80%, 85%, 90%, 95%, 97%, 99%, or 100% identical to any one of SEQ ID NOS:1185, 1816, 1213, 1819, 2083, 1215, 1216, 1811, and 314. In some embodiments, the inhibitory nucleic acid comprises a guide strand sequence comprising or consisting of at least 15, 16, 17, 18, 19, 20, 21, or 22 contiguous nucleotides of a sequence of any one of SEQ ID NOS:1185, 1816, 1213, 1819, 2083, 1215, 1216, 1811, and 314, preferably wherein the guide strand sequence retains positions 2-7 (“seed sequence”) of the selected SEQ ID NO. In some embodiments, the inhibitory nucleic acid comprises a guide strand sequence comprising or consisting of a sequence of any one of SEQ ID NOS:1185, 1816, 1213, 1819, 2083, 1215, 1216, 1811, and 314, wherein 1, 2, 3, or 4 nucleotides at positions 19-22 differ from the selected SEQ ID NO (variant nucleotide(s)) such that the guide strand sequence is no longer complementary to the ATXN2 target sequence at the variant nucleotide(s).

In some embodiments, the inhibitory nucleic acid comprises a guide strand sequence comprising or consisting of the nucleic acid sequence set forth in any one of SEQ ID NOS:1185, 1816, 1213, and 1811. In some embodiments, the inhibitory nucleic acid comprises a guide strand sequence comprising or consisting of the nucleic acid sequence set forth in any one of SEQ ID NOS:1185, 1816, 1213, and 1811, with at least 1, 2, 3, 4, or 5 mismatches to the target ATXN2 mRNA sequence. In some embodiments, the inhibitory nucleic acid comprises a guide strand sequence comprising or consisting of a nucleic acid sequence that is at least 60%, 70%, 75%, 80%, 85%, 90%, 95%, 97%, 99%, or 100% identical to any one of SEQ ID NOS:1185, 1816, 1213, and 1811. In some embodiments, the inhibitory nucleic acid comprises a guide strand sequence comprising or consisting of at least 15, 16, 17, 18, 19, 20, 21, or 22 contiguous nucleotides of a sequence of any one of SEQ ID NOS:1185, 1816, 1213, and 1811, preferably wherein the guide strand sequence retains positions 2-7 (“seed sequence”) of the selected SEQ ID NO. In some embodiments, the inhibitory nucleic acid comprises a guide strand sequence comprising or consisting of a sequence of any one of SEQ ID NOS:1185, 1816, 1213, and 1811, wherein 1, 2, 3, or 4 nucleotides at positions 19-22 differ from the selected SEQ ID NO (variant nucleotide(s)) such that the guide strand sequence is no longer complementary to the ATXN2 target sequence at the variant nucleotide(s).

In some embodiments, the inhibitory nucleic acid is an isolated siRNA duplex that targets ATXN2 mRNA to interfere with ATXN2 expression by mRNA degradation or translational inhibition. A siRNA duplex is a short, double stranded RNA comprising a guide strand, which is complementary to the target ATXN2 mRNA, and a passenger strand, which is homologous to the target ATNX2 mRNA. The guide strand and passenger strand hybridize together to form a duplex structure, and the guide strand has sufficient complementarity to the ATXN2 mRNA sequence to direct ATXN2-specific RNA interference. The guide strand of the siRNA duplex may be about 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides in length or 18-30, 18-29, 18-28, 18-27, 18-26, 18-25, 18-24, 18-23, 18-22, 18-20, 19-30, 19-29, 19-28, 19-27, 19-26, 19-25, 19-24, 19-23, 19-22, 19-21, 20-30, 20-29, 20-28, 20-27, 20-26, 20-25, 20-24, 20-23, 20-22, 21-30, 21-29, 21-28, 21-27, 21-26, 21-25, 21-24, 21-23, 22-30, 22-29, 22-28, 22-27, 22-26, 22-24, 23-30, 23-29, 23-28, 23-27, 23-26, 23-25, 24-30, 24-29, 24-28, 24-27, 24-26, 25-30, 25-29, 25-28, 25-27, 26-30, 26-29, 26-28, 27-30, 27-29, 28-30 nucleotides in length. The passenger strand of the siRNA duplex may be about 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides in length or 18-30, 18-29, 18-28, 18-27, 18-26, 18-25, 18-24, 18-23, 18-22, 18-20, 19-30, 19-29, 19-28, 19-27, 19-26, 19-25, 19-24, 19-23, 19-22, 19-21, 20-30, 20-29, 20-28, 20-27, 20-26, 20-25, 20-24, 20-23, 20-22, 21-30, 21-29, 21-28, 21-27, 21-26, 21-25, 21-24, 21-23, 22-30, 22-29, 22-28, 22-27, 22-26, 22-24, 23-30, 23-29, 23-28, 23-27, 23-26, 23-25, 24-30, 24-29, 24-28, 24-27, 24-26, 25-30, 25-29, 25-28, 25-27, 26-30, 26-29, 26-28, 27-30, 27-29, 28-30 nucleotides in length. In some embodiments, the siRNA duplex contains 2 or 3 nucleotide 3′ overhangs on each strand. In some embodiments, the 3′ overhangs are complementary to the ATXN2 transcript. In some embodiments, the guide strand and passenger strand of the siRNA duplex are at least 60%, 65%, 70%, 75%, 80%, 85%, 90%, 95%, 97%, or 100% complementary to each other, not including any nucleotides in overhang(s).

In some embodiments, the siRNA duplex comprises a guide strand sequence comprising or consisting of the nucleic acid sequence set forth in any one of the guide sequences of Tables 1, 3, 9, 11, 12, 13, 19, 23, 24, and 25, e.g., any one of SEQ ID NOS: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 1176-1288, 1811-1827, 2015, 2065, 2083, 2152, 2203, and 2209.

In some embodiments, the siRNA duplex comprises a guide strand sequence comprising or consisting of the nucleic acid sequence set forth in any one of the guide sequences of Tables 1, 3, 9, 11, 12, 13, 19, 23, 24, and 25, e.g., any one of SEQ ID NOS: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 1176-1288, 1811-1827, 2015, 2065, 2083, 2152, 2203, and 2209, with at least 1, 2, 3, 4, or 5 mismatches to the target ATXN2 mRNA sequence.

In some embodiments, the siRNA duplex comprises a guide strand sequence comprising or consisting of a nucleic acid sequence that is at least 60%, 70%, 75%, 80%, 85%, 90%, 95%, 97%, 99%, or 100% identical to any one of the guide sequences of Tables 1, 3, 9, 11, 12, 13, 19, 23, 24, and 25, e.g., any one of SEQ ID NOS: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 1176-1288, 1811-1827, 2015, 2065, 2083, 2152, 2203, and 2209.

In some embodiments, the siRNA duplex comprises a guide strand sequence comprising or consisting of at least 15, 16, 17, 18, 19, 20, 21, or 22 contiguous nucleotides of a sequence of any one of the guide sequences of Tables 1, 3, 9, 11, 12, 13, 19, 23, 24, and 25, e.g., any one of SEQ ID NOS: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 1176-1288, 1811-1827, 2015, 2065, 2083, 2152, 2203, and 2209, preferably wherein the guide strand sequence retains positions 2-7 (“seed sequence”) of the selected SEQ ID NO.

In some embodiments, the siRNA duplex comprises a guide strand sequence comprising or consisting of a sequence of any one of the guide sequences of Tables 1, 3, 9, 11, 12, 13, 19, 23, 24, and 25, e.g., any one of SEQ ID NOS: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 1176-1288, 1811-1827, 2015, 2065, 2083, 2152, 2203, and 2209, wherein 1, 2, 3, or 4 nucleotides at positions 19-22 differ from the selected SEQ ID NO (variant nucleotide(s)), such that the guide strand sequence is no longer complementary to the ATXN2 target sequence at the variant nucleotide(s).

In some embodiments, the siRNA duplex comprises a guide strand sequence of Table 12. In some embodiments, the siRNA duplex comprises a guide strand sequence comprising or consisting of the nucleic acid sequence set forth in any one of SEQ ID NOS: 12, 14, 40, 60, 100, 104, 108, 112, 124, 126, 128, 166, 198, 220, 242, 302, 306, 308, 330, 336, and 362. In some embodiments, the siRNA duplex comprises a guide strand sequence comprising or consisting of the nucleic acid sequence set forth in any one of SEQ ID NOS: 12, 14, 40, 60, 100, 104, 108, 112, 124, 126, 128, 166, 198, 220, 242, 302, 306, 308, 330, 336, and 362 with at least 1, 2, 3, 4, or 5 mismatches to the target ATXN2 mRNA sequence. In some embodiments, the siRNA duplex comprises a guide strand sequence comprising or consisting of a nucleic acid sequence that is at least 60%, 70%, 75%, 80%, 85%, 90%, 95%, 97%, 99%, or 100% identical to any one of SEQ ID NOS: 12, 14, 40, 60, 100, 104, 108, 112, 124, 126, 128, 166, 198, 220, 242, 302, 306, 308, 330, 336, and 362. In some embodiments, the siRNA duplex comprises a guide strand sequence comprising or consisting of at least 15, 16, 17, 18, 19, 20, 21, or 22 contiguous nucleotides of a sequence of any one of SEQ ID NOS: 12, 14, 40, 60, 100, 104, 108, 112, 124, 126, 128, 166, 198, 220, 242, 302, 306, 308, 330, 336, and 362, preferably wherein the guide strand sequence retains positions 2-7 (“seed sequence”) of the selected SEQ ID NO. In some embodiments, the siRNA duplex comprises a guide strand sequence comprising or consisting of a sequence of any one of SEQ ID NOS: 12, 14, 40, 60, 100, 104, 108, 112, 124, 126, 128, 166, 198, 220, 242, 302, 306, 308, 330, 336, and 362, wherein 1, 2, 3, or 4 nucleotides at positions 19-22 differ from the selected SEQ ID NO (variant nucleotide(s)), such that the guide strand sequence is no longer complementary to the ATXN2 target sequence at the variant nucleotide(s).

In some embodiments, the siRNA duplex comprises a guide strand sequence of Table 13. In some embodiments, the siRNA duplex comprises a guide strand sequence comprising or consisting of the nucleic acid sequence set forth in any one of SEQ ID NOS: 14, 40, 100, 108, 112, 128, 166, 198, 242, 308, 336, and 362. In some embodiments, the siRNA duplex comprises a guide strand sequence comprising or consisting of the nucleic acid sequence set forth in any one of SEQ ID NOS: 14, 40, 100, 108, 112, 128, 166, 198, 242, 308, 336, and 362 with at least 1, 2, 3, 4, or 5 mismatches to the target ATXN2 mRNA sequence. In some embodiments, the siRNA duplex comprises a guide strand sequence comprising or consisting of a nucleic acid sequence that is at least 60%, 70%, 75%, 80%, 85%, 90%, 95%, 97%, 99%, or 100% identical to any one of SEQ ID NOS: 14, 40, 100, 108, 112, 128, 166, 198, 242, 308, 336, and 362. In some embodiments, the siRNA duplex comprises a guide strand sequence comprising or consisting of at least 15, 16, 17, 18, 19, 20, 21, or 22 contiguous nucleotides of a sequence of any one of SEQ ID NOS: 14, 40, 100, 108, 112, 128, 166, 198, 242, 308, 336, and 362, preferably wherein the guide strand sequence retains positions 2-7 (“seed sequence”) of the selected SEQ ID NO. In some embodiments, the siRNA duplex comprises a guide strand sequence comprising or consisting of a sequence of any one of SEQ ID NOS: 14, 40, 100, 108, 112, 128, 166, 198, 242, 308, 336, and 362, wherein 1, 2, 3, or 4 nucleotides at positions 19-22 differ from the selected SEQ ID NO (variant nucleotide(s)), such that the guide strand sequence is no longer complementary to the ATXN2 target sequence at the variant nucleotide(s).

In some embodiments, the siRNA duplex comprises a guide strand sequence of Table 19. In some embodiments, the siRNA duplex comprises a guide strand sequence comprising or consisting of the nucleic acid sequence set forth in any one of SEQ ID NOS:1176-1288, 40, 108, and 166. In some embodiments, the siRNA duplex comprises a guide strand sequence comprising or consisting of the nucleic acid sequence set forth in any one of SEQ ID NOS:1176-1288, 40, 108, and 166, with at least 1, 2, 3, 4, or 5 mismatches to the target ATXN2 mRNA sequence. In some embodiments, the siRNA duplex comprises a guide strand sequence comprising or consisting of a nucleic acid sequence that is at least 60%, 70%, 75%, 80%, 85%, 90%, 95%, 97%, 99%, or 100% identical to any one of SEQ ID NOS:1176-1288, 40, 108, and 166. In some embodiments, the siRNA duplex comprises a guide strand sequence comprising or consisting of at least 15, 16, 17, 18, 19, 20, 21, or 22 contiguous nucleotides of a sequence of any one of SEQ ID NOS:1176-1288, 40, 108, and 166, preferably wherein the guide strand sequence retains positions 2-7 (“seed sequence”) of the selected SEQ ID NO. In some embodiments, the siRNA duplex comprises a guide strand sequence comprising or consisting of a sequence of any one of SEQ ID NOS:1176-1288, 40, 108, and 166, wherein 1, 2, 3, or 4 nucleotides at positions 19-22 differ from the selected SEQ ID NO (variant nucleotide(s)), such that the guide strand sequence is no longer complementary to the ATXN2 target sequence at the variant nucleotide(s).

In some embodiments, the siRNA duplex comprises a guide strand sequence of Table 23. In some embodiments, the siRNA duplex comprises a guide strand sequence comprising or consisting of the nucleic acid sequence set forth in any one of SEQ ID NOS:1908-2007. In some embodiments, the siRNA duplex comprises a guide strand sequence comprising or consisting of the nucleic acid sequence set forth in any one of SEQ ID NOS:1908-2007, with at least 1, 2, 3, 4, or 5 mismatches to the target ATXN2 mRNA sequence. In some embodiments, the siRNA duplex comprises a guide strand sequence comprising or consisting of a nucleic acid sequence that is at least 60%, 70%, 75%, 80%, 85%, 90%, 95%, 97%, 99%, or 100% identical to any one of SEQ ID NOS:1908-2007. In some embodiments, the siRNA duplex comprises a guide strand sequence comprising or consisting of at least 15, 16, 17, 18, 19, 20, 21, or 22 contiguous nucleotides of a sequence of any one of SEQ ID NOS:1908-2007, preferably wherein the guide strand sequence retains positions 2-7 (“seed sequence”) of the selected SEQ ID NO. In some embodiments, the siRNA duplex comprises a guide strand sequence comprising or consisting of a sequence of any one of SEQ ID NOS:1908-2007, wherein 1, 2, 3, or 4 nucleotides at positions 19-22 differ from the selected SEQ ID NO (variant nucleotide(s)), such that the guide strand sequence is no longer complementary to the ATXN2 target sequence at the variant nucleotide(s).

In some embodiments, the siRNA duplex comprises a guide strand sequence of Table 24. In some embodiments, the siRNA duplex comprises a guide strand sequence comprising or consisting of the nucleic acid sequence set forth in any one of SEQ ID NOS:100, 112, 166, 202, 246, 306, 308, 314, 1180, 1185, 1196, 1200, 1211, 1213, 1215, 1216, 1224, 1811-1822, 1824-1827, 2015, 2065, 2083, 2152, 2203, and 2209. In some embodiments, the siRNA duplex comprises a guide strand sequence comprising or consisting of the nucleic acid sequence set forth in any one of SEQ ID NOS:100, 112, 166, 202, 246, 306, 308, 314, 1180, 1185, 1196, 1200, 1211, 1213, 1215, 1216, 1224, 1811-1822, 1824-1827, 2015, 2065, 2083, 2152, 2203, and 2209, with at least 1, 2, 3, 4, or 5 mismatches to the target ATXN2 mRNA sequence. In some embodiments, the siRNA duplex comprises a guide strand sequence comprising or consisting of a nucleic acid sequence that is at least 60%, 70%, 75%, 80%, 85%, 90%, 95%, 97%, 99%, or 100% identical to any one of SEQ ID NOS:100, 112, 166, 202, 246, 306, 308, 314, 1180, 1185, 1196, 1200, 1211, 1213, 1215, 1216, 1224, 1811-1822, 1824-1827, 2015, 2065, 2083, 2152, 2203, and 2209. In some embodiments, the siRNA duplex comprises a guide strand sequence comprising or consisting of at least 15, 16, 17, 18, 19, 20, 21, or 22 contiguous nucleotides of a sequence of any one of SEQ ID NOS:100, 112, 166, 202, 246, 306, 308, 314, 1180, 1185, 1196, 1200, 1211, 1213, 1215, 1216, 1224, 1811-1822, 1824-1827, 2015, 2065, 2083, 2152, 2203, and 2209, preferably wherein the guide strand sequence retains positions 2-7 (“seed sequence”) of the selected SEQ ID NO. In some embodiments, the siRNA duplex comprises a guide strand sequence comprising or consisting of a sequence of any one of SEQ ID NOS:100, 112, 166, 202, 246, 306, 308, 314, 1180, 1185, 1196, 1200, 1211, 1213, 1215, 1216, 1224, 1811-1822, 1824-1827, 2015, 2065, 2083, 2152, 2203, and 2209, wherein 1, 2, 3, or 4 nucleotides at positions 19-22 differ from the selected SEQ ID NO (variant nucleotide(s)), such that the guide strand sequence is no longer complementary to the ATXN2 target sequence at the variant nucleotide(s).

In some embodiments, the siRNA duplex comprises a guide strand sequence of Table 25. In some embodiments, the siRNA duplex comprises a guide strand sequence comprising or consisting of the nucleic acid sequence set forth in any one of SEQ ID NOS:1185, 1816, 1213, 1819, 2083, 1215, 1216, 1811, and 314. In some embodiments, the siRNA duplex comprises a guide strand sequence comprising or consisting of the nucleic acid sequence set forth in any one of SEQ ID NOS:1185, 1816, 1213, 1819, 2083, 1215, 1216, 1811, and 314, with at least 1, 2, 3, 4, or 5 mismatches to the target ATXN2 mRNA sequence. In some embodiments, the siRNA duplex comprises a guide strand sequence comprising or consisting of a nucleic acid sequence that is at least 60%, 70%, 75%, 80%, 85%, 90%, 95%, 97%, 99%, or 100% identical to any one of SEQ ID NOS:1185, 1816, 1213, 1819, 2083, 1215, 1216, 1811, and 314. In some embodiments, the siRNA duplex comprises a guide strand sequence comprising or consisting of at least 15, 16, 17, 18, 19, 20, 21, or 22 contiguous nucleotides of a sequence of any one of SEQ ID NOS:1185, 1816, 1213, 1819, 2083, 1215, 1216, 1811, and 314, preferably wherein the guide strand sequence retains positions 2-7 (“seed sequence”) of the selected SEQ ID NO. In some embodiments, the siRNA duplex comprises a guide strand sequence comprising or consisting of a sequence of any one of SEQ ID NOS:1185, 1816, 1213, 1819, 2083, 1215, 1216, 1811, and 314, wherein 1, 2, 3, or 4 nucleotides at positions 19-22 differ from the selected SEQ ID NO (variant nucleotide(s)), such that the guide strand sequence is no longer complementary to the ATXN2 target sequence at the variant nucleotide(s).

In some embodiments, the siRNA duplex comprises a guide strand sequence comprising or consisting of the nucleic acid sequence set forth in any one of SEQ ID NOS:1185, 1816, 1213, and 1811. In some embodiments, the siRNA duplex comprises a guide strand sequence comprising or consisting of the nucleic acid sequence set forth in any one of SEQ ID NOS:1185, 1816, 1213, and 1811, with at least 1, 2, 3, 4, or 5 mismatches to the target ATXN2 mRNA sequence. In some embodiments, the siRNA duplex comprises a guide strand sequence comprising or consisting of a nucleic acid sequence that is at least 60%, 70%, 75%, 80%, 85%, 90%, 95%, 97%, 99%, or 100% identical to any one of SEQ ID NOS:1185, 1816, 1213, and 1811. In some embodiments, the siRNA duplex comprises a guide strand sequence comprising or consisting of at least 15, 16, 17, 18, 19, 20, 21, or 22 contiguous nucleotides of a sequence of any one of SEQ ID NOS:1185, 1816, 1213, and 1811, preferably wherein the guide strand sequence retains positions 2-7 (“seed sequence”) of the selected SEQ ID NO. In some embodiments, the siRNA duplex comprises a guide strand sequence comprising or consisting of a sequence of any one of SEQ ID NOS:1185, 1816, 1213, and 1811, wherein 1, 2, 3, or 4 nucleotides at positions 19-22 differ from the selected SEQ ID NO (variant nucleotide(s)), such that the guide strand sequence is no longer complementary to the ATXN2 target sequence at the variant nucleotide(s).

In some embodiments the siRNA duplex comprises a guide strand sequence and passenger strand sequence of any one of siRNA duplexes provided by Tables 1, 19, 23, and 24. In some embodiments, the siRNA duplex comprises a guide strand sequence and passenger strand sequence comprising any one of: SEQ ID NOS:12 and 11; SEQ ID NOS: 14 and 13; SEQ ID NOS: 40 and 39; SEQ ID NOS: 60 and 59; SEQ ID NOS: 100 and 99; SEQ ID NOS: 104 and 103; SEQ ID NOS: 108 and 107; SEQ ID NOS: 112 and 111; SEQ ID NOS: 124 and 123; SEQ ID NOS: 126 and 125; SEQ ID NOS: 128 and 127; SEQ ID NOS: 166 and 165; SEQ ID NOS: 198 and 197; SEQ ID NOS: 220 and 219; SEQ ID NOS: 242 and 241; SEQ ID NOS: 302 and 301; SEQ ID NOS: 306 and 305; SEQ ID NOS: 308 and 307; SEQ ID NOS: 330 and 320; SEQ ID NOS: 336 and 335; and SEQ ID NOS: 362 and 361. In some embodiments, the siRNA duplex comprises a guide strand sequence and passenger strand sequence comprising any one of: SEQ ID NOS: 14 and 13; SEQ ID NOS: 40 and 39; SEQ ID NOS: 100 and 99; SEQ ID NOS: 8 and 107: SEQ ID NOS: 2 and 11; SEQ ID NOS 128 and 127; SEQ ID NOS: 166 and 165; SEQ ID NOS: 198 and 197; SEQ ID NOS: 242 and 241; SEQ ID NOS: 308 and 307; SEQ ID NOS: 336 and 335; and SEQ ID NOS: 362 and 361.

TABLE 1
ATXN2 Specific siRNA Duplex Sequences

| | sense sequence | antisense sequence |
|---|---|---|
| duplex ID | (passenger) | (guide) |
| XD-14738 | CCUCCGCCUCAGACUGUUUUGG | AAAACAGUCUGAGGCGGAGGGA |
| | [SEQ ID NO: 3] | [SEQ ID NO: 4] |
| XD-14739 | CUCCGCCUCAGACUGUUUUAGU | UAAAACAGUCUGAGGCGGAGGG |
| | [SEQ ID NO: 5] | [SEQ ID NO: 6] |
| XD-14740 | CGGCGGCAGCGGCCUUCUAACG | UUAGAAGGCCGCUGCCGCCGGG |
| | [SEQ ID NO: 7] | [SEQ ID NO: 8] |
| XD-14741 | GGACUGCCUCAGUCUACGAUUU | AUCGUAGACUGAGGCAGUCCUU |
| | [SEQ ID NO: 9] | [SEQ ID NO: 10] |
| XD-14742 | CUGCCUCAGUCUACGAUUUAUU | UAAAUCGUAGACUGAGGCAGUC |
| | [SEQ ID NO: 11] | [SEQ ID NO: 12] |
| XD-14743 | UGCCUCAGUCUACGAUUUCUUU | AGAAAUCGUAGACUGAGGCAGU |
| | [SEQ ID NO: 13] | [SEQ ID NO: 14] |
| XD-14744 | CCUCAGUCUACGAUUUCUUUUG | AAAGAAAUCGUAGACUGAGGCA |
| | [SEQ ID NO: 15] | [SEQ ID NO: 16] |
| XD-14745 | CAGUCUACGAUUUCUUUUGAUG | UCAAAAGAAAUCGUAGACUGAG |
| | [SEQ ID NO: 17] | [SEQ ID NO: 18] |
| XD-14746 | GAGGAUGGUUCAUAUACUUACA | UAAGUAUAUGAACCAUCCUCAU |
| | [SEQ ID NO: 19] | [SEQ ID NO: 20] |
| XD-14747 | AGGAUGGUUCAUAUACUUAAAU | UUAAGUAUAUGAACCAUCCUCA |
| | [SEQ ID NO: 21] | [SEQ ID NO: 22] |
| XD-14748 | GGAUGGUUCAUAUACUUACAUC | UGUAAGUAUAUGAACCAUCCUC |
| | [SEQ ID NO: 23] | [SEQ ID NO: 24] |
| XD-14749 | UUCAUAUACUUACAUCAGUUGU | AACUGAUGUAAGUAUAUGAACC |
| | [SEQ ID NO: 25] | [SEQ ID NO: 26] |
| XD-14750 | AUGAGAAAAGUACAGAAUCAAG | UGAUUCUGUACUUUUCUCAUGU |
| | [SEQ ID NO: 27] | [SEQ ID NO: 28] |
| XD-14751 | GAGAAAAGUACAGAAUCCAAUU | UUGGAUUCUGUACUUUUCUCAU |
| | [SEQ ID NO: 29] | [SEQ ID NO: 30] |
| XD-14752 | AAAGUACAGAAUCCAGUUCAGG | UGAACUGGAUUCUGUACUUUUC |
| | [SEQ ID NO: 31] | [SEQ ID NO: 32] |
| XD-14753 | AAGUACAGAAUCCAGUUCGAGG | UCGAACUGGAUUCUGUACUUUU |
| | [SEQ ID NO: 33] | [SEQ ID NO: 34] |
| XD-14754 | GUACAGAAUCCAGUUCGGGACC | UCCCGAACUGGAUUCUGUACUU |
| | [SEQ ID NO: 35] | [SEQ ID NO: 36] |
| XD-14755 | UACAGAAUCCAGUUCGGGGACG | UCCCCGAACUGGAUUCUGUACU |
| | [SEQ ID NO: 37] | [SEQ ID NO: 38] |
| XD-14756 | UCAGACUUUGUUGUGGUACAGU | UGUACCACAACAAAGUCUGAAC |
| | [SEQ ID NO: 39] | [SEQ ID NO: 40] |
| XD-14757 | UUUGUUGUGGUACAGUUUAAAG | UUAAACUGUACCACAACAAAGU |
| | [SEQ ID NO: 41] | [SEQ ID NO: 42] |
| XD-14758 | UUGUUGUGGUACAGUUUAAAGA | UUUAAACUGUACCACAACAAAG |
| | [SEQ ID NO: 43] | [SEQ ID NO: 44] |
| XD-14759 | UGUGGUACAGUUUAAAGAUAUG | UAUCUUUAAACUGUACCACAAC |
| | [SEQ ID NO: 45] | [SEQ ID NO: 46] |
| XD-14760 | UUACUGACUCUGCUAUCAGUGC | ACUGAUAGCAGAGUCAGUAAAA |
| | [SEQ ID NO: 47] | [SEQ ID NO: 48] |
| XD-14761 | CUGACUCUGCUAUCAGUGCUAA | AGCACUGAUAGCAGAGUCAGUA |
| | [SEQ ID NO: 49] | [SEQ ID NO: 50] |
| XD-14762 | UGACUCUGCUAUCAGUGCUAAA | UAGCACUGAUAGCAGAGUCAGU |
| | [SEQ ID NO: 51] | [SEQ ID NO: 52] |
| XD-14763 | CUAAAGUGAAUGGCGAACAAAA | UUGUUCGCCAUUCACUUUAGCA |
| | [SEQ ID NO: 53] | [SEQ ID NO: 54] |
| XD-14764 | AAAGUGAAUGGCGAACACAAAG | UUGUGUUCGCCAUUCACUUUAG |
| | [SEQ ID NO: 55] | [SEQ ID NO: 56] |
| XD-14765 | GUGAAUGGCGAACACAAAGAGA | UCUUUGUGUUCGCCAUUCACUU |
| | [SEQ ID NO: 57] | [SEQ ID NO: 58] |
| XD-14766 | CUUUGGAAAAUGACGUAUCUAA | AGAUACGUCAUUUUCCAAAGCC |
| | [SEQ ID NO: 59] | [SEQ ID NO: 60] |
| XD-14767 | UUGGAAAAUGACGUAUCUAAUG | UUAGAUACGUCAUUUUCCAAAG |
| | [SEQ ID NO: 61] | [SEQ ID NO: 62] |
| XD-14768 | GGAAAAUGACGUAUCUAAUAGA | UAUUAGAUACGUCAUUUUCCAA |
| | [SEQ ID NO: 63] | [SEQ ID NO: 64] |
| XD-14769 | AAAUGACGUAUCUAAUGGAUGG | AUCCAUUAGAUACGUCAUUUUC |
| | [SEQ ID NO: 65] | [SEQ ID NO: 66] |
| XD-14770 | AUGACGUAUCUAAUGGAUGAGA | UCAUCCAUUAGAUACGUCAUUU |
| | [SEQ ID NO: 67] | [SEQ ID NO: 68] |
| XD-14771 | UAAUGGAUGGGAUCCCAAUAAU | UAUUGGGAUCCCAUCCAUUAGA |
| | [SEQ ID NO: 69] | [SEQ ID NO: 70] |
| XD-14772 | AAUGGAUGGGAUCCCAAUGAUA | UCAUUGGGAUCCCAUCCAUUAG |
| | [SEQ ID NO: 71] | [SEQ ID NO: 72] |
| XD-14773 | AUAUGUUUCGAUAUAAUGAAGA | UUCAUUAUAUCGAAACAUAUCA |
| | [SEQ ID NO: 73] | [SEQ ID NO: 74] |
| XD-14774 | AUGUUUCGAUAUAAUGAAGAAA | UCUUCAUUAUAUCGAAACAUAU |
| | [SEQ ID NO: 75] | [SEQ ID NO: 76] |
| XD-14775 | GUCUACGUAUGAUAGCAGUUUA | AACUGCUAUCAUACGUAGACAC |
| | [SEQ ID NO: 77] | [SEQ ID NO: 78] |
| XD-14776 | CGUAUACAGUGCCCUUAGAAAG | UUCUAAGGGCACUGUAUACGAA |
| | [SEQ ID NO: 79] | [SEQ ID NO: 80] |
| XD-14777 | UAUACAGUGCCCUUAGAAAAAG | UUUUCUAAGGGCACUGUAUACG |
| | [SEQ ID NO: 81] | [SEQ ID NO: 82] |
| XD-14778 | CAAGGGCAAACCAGUUAGCAGA | UGCUAACUGGUUUGCCCUUGCU |
| | [SEQ ID NO: 83] | [SEQ ID NO: 84] |
| XD-14779 | AGGGCAAACCAGUUAGCAGAAG | UCUGCUAACUGGUUUGCCCUUG |
| | [SEQ ID NO: 85] | [SEQ ID NO: 86] |
| XD-14780 | UGAGUCAAGUGCCCAGUACAAA | UGUACUGGGCACUUGACUCAAU |
| | [SEQ ID NO: 87] | [SEQ ID NO: 88] |
| XD-14781 | GAGUCAAGUGCCCAGUACAAAG | UUGUACUGGGCACUUGACUCAA |
| | [SEO ID NO: 89] | [SEO ID NO: 90] |
| XD-14782 | GCCCAGUACAAAGCUCGAGUGG | ACUCGAGCUUUGUACUGGGCAC |
| | [SEQ ID NO: 91] | [SEQ ID NO: 92] |
| XD-14783 | AAGAAAAAUACACAGCAGUUCA | AACUGCUGUGUAUUUUUCUUCC |
| | [SEQ ID NO: 93] | [SEQ ID NO: 94] |
| XD-14784 | GAGGGGCACAGCAUAAACAAUA | UUGUUUAUGCUGUGCCCCUCAC |
| | [SEQ ID NO: 95] | [SEQ ID NO: 96] |
| XD-14785 | AGUCAUAUCCUGGGGAAGUAGG | UACUUCCCCAGGAUAUGACUUC |
| | [SEQ ID NO: 97] | [SEQ ID NO: 98] |
| XD-14786 | GAGACAGAAUUCACCGCGUAUG | UACGCGGUGAAUUCUGUCUCCC |
| | [SEQ ID NO: 99] | [SEQ ID NO: 100] |
| XD-14787 | AGACAGAAUUCACCGCGUAUGG | AUACGCGGUGAAUUCUGUCUCC |
| | [SEQ ID NO: 101] | [SEQ ID NO: 102] |
| XD-14788 | GACAGAAUUCACCGCGUAUAGG | UAUACGCGGUGAAUUCUGUCUC |
| | [SEQ ID NO: 103] | [SEQ ID NO: 104] |
| XD-14789 | ACAGAAUUCACCGCGUAUGAGC | UCAUACGCGGUGAAUUCUGUCU |
| | [SEQ ID NO: 105] | [SEQ ID NO: 106] |
| XD-14790 | ACUUCAGAUUUCAACCCGAAUU | UUCGGGUUGAAAUCUGAAGUGU |
| | [SEQ ID NO: 107] | [SEQ ID NO: 108] |
| XD-14791 | UCAGACCAAAGAGUAGUUAAUG | UUAACUACUCUUUGGUCUGAAC |
| | [SEQ ID NO: 109] | [SEQ ID NO: 110] |
| XD-14792 | CAGACCAAAGAGUAGUUAAUGG | AUUAACUACUCUUUGGUCUGAA |
| | [SEQ ID NO: 111] | [SEQ ID NO: 112] |
| XD-14793 | AGACCAAAGAGUAGUUAAUAGA | UAUUAACUACUCUUUGGUCUGA |
| | [SEQ ID NO: 113] | [SEQ ID NO: 114] |
| XD-14794 | GACCAAAGAGUAGUUAAUGAAG | UCAUUAACUACUCUUUGGUCUG |
| | [SEQ ID NO: 115] | [SEQ ID NO: 116] |
| XD-14795 | CCAAAGAGUAGUUAAUGGAAGU | UUCCAUUAACUACUCUUUGGUC |
| | [SEQ ID NO: 117] | [SEQ ID NO: 118] |
| XD-14796 | UGUCCCCAAAGGCCCAGCGACA | UCGCUGGGCCUUUGGGGACAUC |
| | [SEQ ID NO: 119] | [SEQ ID NO: 120] |
| XD-14797 | GCCCAGCGACAUCCUCGAAAUC | UUUCGAGGAUGUCGCUGGGCCU |
| | [SEQ ID NO: 121] | [SEQ ID NO: 122] |
| XD-14798 | CCCAGCGACAUCCUCGAAAUCA | AUUUCGAGGAUGUCGCUGGGCC |
| | [SEQ ID NO: 123] | [SEQ ID NO: 124] |
| XD-14799 | CCAGCGACAUCCUCGAAAUAAC | UAUUUCGAGGAUGUCGCUGGGC |
| | [SEQ ID NO: 125] | [SEQ ID NO: 126] |
| XD-14800 | AGCGACAUCCUCGAAAUCAAAG | UUGAUUUCGAGGAUGUCGCUGG |
| | [SEQ ID NO: 127] | [SEQ ID NO: 128] |
| XD-14801 | GCGACAUCCUCGAAAUCACAGA | UGUGAUUUCGAGGAUGUCGCUG |
| | [SEQ ID NO: 129] | [SEQ ID NO: 130] |
| XD-14802 | UGCUGGGAGGGGUUCCAUAUCC | AUAUGGAACCCCUCCCAGCAGA |
| | [SEQ ID NO: 131] | [SEQ ID NO: 132] |
| XD-14803 | GCUACUCCUCCAGUAGCAAAGA | UUUGCUACUGGAGGAGUAGCUG |
| | [SEQ ID NO: 133] | [SEQ ID NO: 134] |
| XD-14804 | CCUCCAGUAGCAAGGACCAAUC | UUGGUCCUUGCUACUGGAGGAG |
| | [SEQ ID NO: 135] | [SEQ ID NO: 136] |
| XD-14805 | GUGGUCAGUGGGGUUCCAAAAU | UUUGGAACCCCACUGACCACUG |
| | [SEQ ID NO: 137] | [SEQ ID NO: 138] |
| XD-14806 | GUGGGGUUCCAAGAUUAUCACC | UGAUAAUCUUGGAACCCCACUG |
| | [SEQ ID NO: 139] | [SEQ ID NO: 140] |
| XD-14807 | UCCAAGAUUAUCCCCUAAAACU | UUUUAGGGGAUAAUCUUGGAAC |
| | [SEQ ID NO: 141] | [SEQ ID NO: 142] |
| XD-14808 | CCAAGAUUAUCCCCUAAAAAUC | UUUUUAGGGGAUAAUCUUGGAA |
| | [SEQ ID NO: 143] | [SEQ ID NO: 144] |
| XD-14809 | CAAGAUUAUCCCCUAAAACUCA | AGUUUUAGGGGAUAAUCUUGGA |
| | [SEQ ID NO: 145] | [SEQ ID NO: 146] |
| XD-14810 | AAGAUUAUCCCCUAAAACUAAU | UAGUUUUAGGGGAUAAUCUUGG |
| | [SEQ ID NO: 147] | [SEQ ID NO: 148] |
| XD-14811 | AGAUUAUCCCCUAAAACUCAUA | UGAGUUUUAGGGGAUAAUCUUG |
| | [SEQ ID NO: 149] | [SEQ ID NO: 150] |
| XD-14812 | GAUUAUCCCCUAAAACUCAUAG | AUGAGUUUUAGGGGAUAAUCUU |
| | [SEQ ID NO: 151] | [SEQ ID NO: 152] |
| XD-14813 | AUUAUCCCCUAAAACUCAUAGA | UAUGAGUUUUAGGGGAUAAUCU |
| | [SEQ ID NO: 153] | [SEQ ID NO: 154] |
| XD-14814 | UAUCCCCUAAAACUCAUAGACC | UCUAUGAGUUUUAGGGGAUAAU |
| | [SEQ ID NO: 155] | [SEQ ID NO: 156] |
| XD-14815 | AACUCAUAGACCCAGGUCUACC | UAGACCUGGGUCUAUGAGUUUU |
| | [SEQ ID NO: 157] | [SEQ ID NO: 158] |
| XD-14816 | UCCCCAAGCUGGUAUUAUUACA | UAAUAAUACCAGCUUGGGGAGA |
| | [SEQ ID NO: 159] | [SEQ ID NO: 160] |
| XD-14817 | CCCCAAGCUGGUAUUAUUCAAA | UGAAUAAUACCAGCUUGGGGAG |
| | [SEQ ID NO: 161] | [SEQ ID NO: 162] |
| XD-14818 | CAUCUCCUACGCCUGCUAGUCC | ACUAGCAGGCGUAGGAGAUGCA |
| | [SEQ ID NO: 163] | [SEQ ID NO: 164] |
| XD-14819 | CCUGCUAGUCCUGCAUCGAACA | UUCGAUGCAGGACUAGCAGGCG |
| | [SEQ ID NO: 165] | [SEQ ID NO: 166] |
| XD-14820 | UGCUAGUCCUGCAUCGAACAGA | UGUUCGAUGCAGGACUAGCAGG |
| | [SEQ ID NO: 167] | [SEQ ID NO: 168] |
| XD-14821 | CUGCAUCGAACAGAGCUGUUAC | AACAGCUCUGUUCGAUGCAGGA |
| | [SEQ ID NO: 169] | [SEQ ID NO: 170] |
| XD-14822 | GCAUCGAACAGAGCUGUUAACC | UUAACAGCUCUGUUCGAUGCAG |
| | [SEQ ID NO: 171] | [SEQ ID NO: 172] |
| XD-14823 | ACCCCUUCUAGUGAGGCUAAAG | UUAGCCUCACUAGAAGGGGUAA |
| | [SEQ ID NO: 173] | [SEQ ID NO: 174] |
| XD-14824 | CCCCUUCUAGUGAGGCUAAAGA | UUUAGCCUCACUAGAAGGGGUA |
| | [SEQ ID NO: 175] | [SEQ ID NO: 176] |
| XD-14825 | CCUUCUAGUGAGGCUAAAGAUU | UCUUUAGCCUCACUAGAAGGGG |
| | [SEQ ID NO: 177] | [SEQ ID NO: 178] |
| XD-14826 | UUCUAGUGAGGCUAAAGAUUCC | AAUCUUUAGCCUCACUAGAAGG |
| | [SEQ ID NO: 179] | [SEQ ID NO: 180] |
| XD-14827 | AAUGAAACAUCACCUAGCUUCU | AAGCUAGGUGAUGUUUCAUUGG |
| | [SEQ ID NO: 181] | [SEQ ID NO: 182] |
| XD-14828 | ACAUCACCUAGCUUCUCAAAAG | UUUGAGAAGCUAGGUGAUGUUU |
| | [SEQ ID NO: 183] | [SEQ ID NO: 184] |
| XD-14829 | AUCACCUAGCUUCUCAAAAACU | UUUUUGAGAAGCUAGGUGAUGU |
| | [SEQ ID NO: 185] | [SEQ ID NO: 186] |
| XD-14830 | CCUAGCUUCUCAAAAGCUGAAA | UCAGCUUUUGAGAAGCUAGGUG |
| | [SEQ ID NO: 187] | [SEQ ID NO: 188] |
| XD-14831 | AAAACAAAGGUAUAUCACCAGU | UGGUGAUAUACCUUUGUUUUCA |
| | [SEQ ID NO: 189] | [SEQ ID NO: 190] |
| XD-14832 | UAAGAAUGAUUUUAGGUUAAAG | UUAACCUAAAAUCAUUCUUAAA |
| | [SEQ ID NO: 191] | [SEQ ID NO: 192] |
| XD-14833 | AAGAAUGAUUUUAGGUUACAGC | UGUAACCUAAAAUCAUUCUUAA |
| | [SEQ ID NO: 193] | [SEQ ID NO: 194] |
| XD-14834 | ACUUCUGAAUCUAUGGAUCAAC | UGAUCCAUAGAUUCAGAAGUAG |
| | [SEQ ID NO: 195] | [SEQ ID NO: 196] |
| XD-14835 | UGAAUCUAUGGAUCAACUAAUA | UUAGUUGAUCCAUAGAUUCAGA |
| | [SEQ ID NO: 197] | [SEQ ID NO: 198] |
| XD-14836 | AUCUAUGGAUCAACUACUAAAC | UUAGUAGUUGAUCCAUAGAUUC |
| | [SEQ ID NO: 199] | [SEQ ID NO: 200] |
| XD-14837 | UCUAUGGAUCAACUACUAAACA | UUUAGUAGUUGAUCCAUAGAUU |
| | [SEQ ID NO: 201] | [SEQ ID NO: 202] |
| XD-14838 | UAUGGAUCAACUACUAAACAAA | UGUUUAGUAGUUGAUCCAUAGA |
| | [SEQ ID NO: 203] | [SEQ ID NO: 204] |
| XD-14839 | AUGGAUCAACUACUAAACAAAA | UUGUUUAGUAGUUGAUCCAUAG |
| | [SEQ ID NO: 205] | [SEQ ID NO: 206] |
| XD-14840 | UGGAUCAACUACUAAACAAAAA | UUUGUUUAGUAGUUGAUCCAUA |
| | [SEQ ID NO: 207] | [SEQ ID NO: 208] |
| XD-14841 | GGAUCAACUACUAAACAAAAAU | UUUUGUUUAGUAGUUGAUCCAU |
| | [SEQ ID NO: 209] | [SEQ ID NO: 210] |
| XD-14842 | GCCGAAUAGCCCCAGCAUUUCC | AAAUGCUGGGGCUAUUCGGCUU |
| | [SEQ ID NO: 211] | [SEQ ID NO: 212] |
| XD-14843 | CAGCAUUUCCCCUUCAAUAAUU | UUAUUGAAGGGGAAAUGCUGGG |
| | [SEQ ID NO: 213] | [SEQ ID NO: 214] |
| XD-14844 | UUUCCCCUUCAAUACUUAGUAA | ACUAAGUAUUGAAGGGGAAAUG |
| | [SEQ ID NO: 215] | [SEQ ID NO: 216] |
| XD-14845 | UUCCCCUUCAAUACUUAGUAAC | UACUAAGUAUUGAAGGGGAAAU |
| | [SEQ ID NO: 217] | [SEQ ID NO: 218] |
| XD-14846 | UCCCCUUCAAUACUUAGUAACA | UUACUAAGUAUUGAAGGGGAAA |
| | [SEQ ID NO: 219] | [SEQ ID NO: 220] |
| XD-14847 | CCCCUUCAAUACUUAGUAAAAC | UUUACUAAGUAUUGAAGGGGAA |
| | [SEQ ID NO: 221] | [SEQ ID NO: 222] |
| XD-14848 | UUCAAUACUUAGUAACACGAAG | UCGUGUUACUAAGUAUUGAAGG |
| | [SEQ ID NO: 223] | [SEQ ID NO: 224] |
| XD-14849 | UCAAUACUUAGUAACACGGAGC | UCCGUGUUACUAAGUAUUGAAG |
| | [SEQ ID NO: 225] | [SEQ ID NO: 226] |
| XD-14850 | AUACUUAGUAACACGGAGCACA | UGCUCCGUGUUACUAAGUAUUG |
| | [SEQ ID NO: 227] | [SEQ ID NO: 228] |
| XD-14851 | GUCACUUCCCAAGGGGUUCAGA | UGAACCCCUUGGGAAGUGACCU |
| | [SEQ ID NO: 229] | [SEQ ID NO: 230] |
| XD-14852 | CCAAGGGGUUCAGACUUCCAGC | UGGAAGUCUGAACCCCUUGGGA |
| | [SEQ ID NO: 231] | [SEQ ID NO: 232] |
| XD-14853 | GACGCAGCUGAGCAAGUUAAGA | UUAACUUGCUCAGCUGCGUCUU |
| | [SEQ ID NO: 233] | [SEQ ID NO: 234] |
| XD-14854 | CUGAGCAAGUUAGGAAAUCAAC | UGAUUUCCUAACUUGCUCAGCU |
| | [SEQ ID NO: 235] | [SEQ ID NO: 236] |
| XD-14855 | GAGCAAGUUAGGAAAUCAAAAU | UUUGAUUUCCUAACUUGCUCAG |
| | [SEQ ID NO: 237] | [SEQ ID NO: 238] |
| XD-14856 | CCAAUGCAAAGGAGUUCAAACC | UUUGAACUCCUUUGCAUUGGGA |
| | [SEQ ID NO: 239] | [SEQ ID NO: 240] |
| XD-14857 | GGAGUUCAACCCACGUUCCUUC | AGGAACGUGGGUUGAACUCCUU |
| | [SEQ ID NO: 241] | [SEQ ID NO: 242] |
| XD-14858 | UCUCAGCCAAAGCCUUCUAAUA | UUAGAAGGCUUUGGCUGAGAGA |
| | [SEQ ID NO: 243] | [SEQ ID NO: 244] |
| XD-14859 | CAGCCAAAGCCUUCUACUAACC | UUAGUAGAAGGCUUUGGCUGAG |
| | [SEQ ID NO: 245] | [SEQ ID NO: 246] |
| XD-14860 | AGCCAAAGCCUUCUACUACACC | UGUAGUAGAAGGCUUUGGCUGA |
| | [SEQ ID NO: 247] | [SEQ ID NO: 248] |
| XD-14861 | GCCAAAGCCUUCUACUACCACA | UGGUAGUAGAAGGCUUUGGCUG |
| | [SEQ ID NO: 249] | [SEQ ID NO: 250] |
| XD-14862 | ACCCCAACUUCACCUCGGCAUC | UGCCGAGGUGAAGUUGGGGUAG |
| | [SEQ ID NO: 251] | [SEQ ID NO: 252] |
| XD-14863 | CCCCAACUUCACCUCGGCCUCA | AGGCCGAGGUGAAGUUGGGGUA |
| | [SEQ ID NO: 253] | [SEQ ID NO: 254] |
| XD-14864 | CCCAACUUCACCUCGGCCUAAA | UAGGCCGAGGUGAAGUUGGGGU |
| | [SEQ ID NO: 255] | [SEQ ID NO: 256] |
| XD-14865 | ACUUCACCUCGGCCUCAAGAAC | UCUUGAGGCCGAGGUGAAGUUG |
| | [SEQ ID NO: 257] | [SEQ ID NO: 258] |
| XD-14866 | ACCUCGGCCUCAAGCACAAACU | UUUGUGCUUGAGGCCGAGGUGA |
| | [SEQ ID NO: 259] | [SEQ ID NO: 260] |
| XD-14867 | CCUCGGCCUCAAGCACAACAUA | UGUUGUGCUUGAGGCCGAGGUG |
| | [SEQ ID NO: 261] | [SEQ ID NO: 262] |
| XD-14868 | AGCACAACCUAGCCCAUCUAUG | UAGAUGGGCUAGGUUGUGCUUG |
| | [SEQ ID NO: 263] | [SEQ ID NO: 264] |
| XD-14869 | GCACAACCUAGCCCAUCUAUGG | AUAGAUGGGCUAGGUUGUGCUU |
| | [SEQ ID NO: 265] | [SEQ ID NO: 266] |
| XD-14870 | ACAACCUAGCCCAUCUAUGAUG | UCAUAGAUGGGCUAGGUUGUGC |
| | [SEQ ID NO: 267] | [SEQ ID NO: 268] |
| XD-14871 | CAACCUAGCCCAUCUAUGGUGG | ACCAUAGAUGGGCUAGGUUGUG |
| | [SEQ ID NO: 269] | [SEQ ID NO: 270] |
| XD-14872 | GCCCAUCUAUGGUGGGUCAUCA | AUGACCCACCAUAGAUGGGCUA |
| | [SEQ ID NO: 271] | [SEQ ID NO: 272] |
| XD-14873 | CCCAUCUAUGGUGGGUCAUAAA | UAUGACCCACCAUAGAUGGGCU |
| | [SEQ ID NO: 273] | [SEQ ID NO: 274] |
| XD-14874 | CCAUCUAUGGUGGGUCAUCAAC | UGAUGACCCACCAUAGAUGGGC |
| | [SEQ ID NO: 275] | [SEQ ID NO: 276] |
| XD-14875 | CAUCUAUGGUGGGUCAUCAACA | UUGAUGACCCACCAUAGAUGGG |
| | [SEQ ID NO: 277] | [SEQ ID NO: 278] |
| XD-14876 | GCCAACUCCAGUUUAUACUAAG | UAGUAUAAACUGGAGUUGGCUG |
| | [SEQ ID NO: 279] | [SEQ ID NO: 280] |
| XD-14877 | CACCAAAUAUGAUGUAUCCAGU | UGGAUACAUCAUAUUUGGUGCA |
| | [SEQ ID NO: 281] | [SEQ ID NO: 282] |
| XD-14878 | AGCCCAGGCGUGCAACCUUUAU | AAAGGUUGCACGCCUGGGCUCA |
| | [SEQ ID NO: 283] | [SEQ ID NO: 284] |
| XD-14879 | UACCCAAUACCUAUGACGCACA | UGCGUCAUAGGUAUUGGGUAUA |
| | [SEQ ID NO: 285] | [SEQ ID NO: 286] |
| XD-14880 | CAAUACCUAUGACGCCCAUACC | UAUGGGCGUCAUAGGUAUUGGG |
| | [SEQ ID NO: 287] | [SEQ ID NO: 288] |
| XD-14881 | AUACCUAUGACGCCCAUGCAAG | UGCAUGGGCGUCAUAGGUAUUG |
| | [SEQ ID NO: 289] | [SEQ ID NO: 290] |
| XD-14882 | GAAUCAAGCCAAGACAUAUAGA | UAUAUGUCUUGGCUUGAUUCAC |
| | [SEQ ID NO: 291] | [SEQ ID NO: 292] |
| XD-14883 | AAUCAAGCCAAGACAUAUAAAG | UUAUAUGUCUUGGCUUGAUUCA |
| | [SEQ ID NO: 293] | [SEQ ID NO: 294] |
| XD-14884 | AUCAAGCCAAGACAUAUAGAGC | UCUAUAUGUCUUGGCUUGAUUC |
| | [SEQ ID NO: 295] | [SEQ ID NO: 296] |
| XD-14885 | AAGCCAAGACAUAUAGAGCAGU | UGCUCUAUAUGUCUUGGCUUGA |
| | [SEQ ID NO: 297] | [SEQ ID NO: 298] |
| XD-14886 | CCAAAUAUGCCCCAACAGCAGC | UGCUGUUGGGGCAUAUUUGGUA |
| | [SEQ ID NO: 299] | [SEQ ID NO: 300] |
| XD-14887 | CAAAUAUGCCCCAACAGCGACA | UCGCUGUUGGGGCAUAUUUGGU |
| | [SEQ ID NO: 301] | [SEQ ID NO: 302] |
| XD-14888 | GCAGCGGGCCCACCGAUUGAAG | UCAAUCGGUGGGCCCGCUGCUG |
| | [SEQ ID NO: 303] | [SEQ ID NO: 304] |
| XD-14889 | ACCAGCUUACUCCACGCAAUAU | AUUGCGUGGAGUAAGCUGGUGG |
| | [SEQ ID NO: 305] | [SEQ ID NO: 306] |
| XD-14890 | CCAGCUUACUCCACGCAAUAUG | UAUUGCGUGGAGUAAGCUGGUG |
| | [SEQ ID NO: 307] | [SEQ ID NO: 308] |
| XD-14891 | AGCUUACUCCACGCAAUAUAUU | UAUAUUGCGUGGAGUAAGCUGG |
| | [SEQ ID NO: 309] | [SEQ ID NO: 310] |
| XD-14892 | UCCACGCAAUAUGUUGCCUACA | UAGGCAACAUAUUGCGUGGAGU |
| | [SEQ ID NO: 311] | [SEQ ID NO: 312] |
| XD-14893 | AGUCUCAGCAUCCUCAUGUAUA | UACAUGAGGAUGCUGAGACUGA |
| | [SEQ ID NO: 313] | [SEQ ID NO: 314] |
| XD-14894 | CAGCAUCCUCAUGUCUAUAAUC | UUAUAGACAUGAGGAUGCUGAG |
| | [SEQ ID NO: 315] | [SEQ ID NO: 316] |
| XD-14895 | GUCCUGUAAUACAGGGUAAUGC | AUUACCCUGUAUUACAGGACUA |
| | [SEQ ID NO: 317] | [SEQ ID NO: 318] |
| XD-14896 | ACAGGGUAAUGCUAGAAUGAUG | UCAUUCUAGCAUUACCCUGUAU |
| | [SEQ ID NO: 319] | [SEQ ID NO: 320] |
| XD-14897 | ACUCAGUACGGGGCUCAUGAGC | UCAUGAGCCCCGUACUGAGUUG |
| | [SEQ ID NO: 321] | [SEQ ID NO: 322] |
| XD-14898 | GAGCAGACGCAUGCGAUGUAUG | UACAUCGCAUGCGUCUGCUCAU |
| | [SEQ ID NO: 323] | [SEQ ID NO: 324] |
| XD-14899 | AGCAGACGCAUGCGAUGUAUGC | AUACAUCGCAUGCGUCUGCUCA |
| | [SEQ ID NO: 325] | [SEQ ID NO: 326] |
| XD-14900 | GCAGACGCAUGCGAUGUAUACA | UAUACAUCGCAUGCGUCUGCUC |
| | [SEQ ID NO: 327] | [SEQ ID NO: 328] |
| XD-14901 | CUUGCUCAGCAGUAUGCGCACC | UGCGCAUACUGCUGAGCAAGGG |
| | [SEQ ID NO: 329] | [SEQ ID NO: 330] |
| XD-14902 | CAGCAGUAUGCGCACCCUAACG | UUAGGGUGCGCAUACUGCUGAG |
| | [SEQ ID NO: 331] | [SEQ ID NO: 332] |
| XD-14903 | CAGUAUGCGCACCCUAACGAUA | UCGUUAGGGUGCGCAUACUGCU |
| | [SEQ ID NO: 333] | [SEQ ID NO: 334] |
| XD-14904 | AGUAUGCGCACCCUAACGCUAC | AGCGUUAGGGUGCGCAUACUGC |
| | [SEQ ID NO: 335] | [SEQ ID NO: 336] |
| XD-14905 | GUAUGCGCACCCUAACGCUACC | UAGCGUUAGGGUGCGCAUACUG |
| | [SEQ ID NO: 337] | [SEQ ID NO: 338] |
| XD-14906 | CAGCAGUCAGCCAUUUACCACG | UGGUAAAUGGCUGACUGCUGCU |
| | [SEQ ID NO: 339] | [SEQ ID NO: 340] |
| XD-14907 | AGCCAUUUACCACGCGGGGAUU | UCCCCGCGUGGUAAAUGGCUGA |
| | [SEQ ID NO: 341] | [SEQ ID NO: 342] |
| XD-14908 | UCCAACACGCAGUCGCCACAGA | UGUGGCGACUGCGUGUUGGAGG |
| | [SEQ ID NO: 343] | [SEQ ID NO: 344] |
| XD-14909 | AACACGCAGUCGCCACAGAAUA | UUCUGUGGCGACUGCGUGUUGG |
| | [SEQ ID NO: 345] | [SEQ ID NO: 346] |
| XD-14910 | ACGCAGUCGCCACAGAAUAAUU | UUAUUCUGUGGCGACUGCGUGU |
| | [SEQ ID NO: 347] | [SEQ ID NO: 348] |
| XD-14911 | GCAGUCGCCACAGAAUAGUUUC | AACUAUUCUGUGGCGACUGCGU |
| | [SEQ ID NO: 349] | [SEQ ID NO: 350] |
| XD-14912 | ACAGAAUAGUUUCCCAGCAACA | UUGCUGGGAAACUAUUCUGUGG |
| | [SEQ ID NO: 351] | [SEQ ID NO: 352] |
| XD-14913 | AGUUUCCCAGCAGCACAACAGA | UGUUGUGCUGCUGGGAAACUAU |
| | [SEQ ID NO: 353] | [SEQ ID NO: 354] |
| XD-14914 | ACGAUCCAUCCUUCUCACGUUC | ACGUGAGAAGGAUGGAUCGUAA |
| | [SEQ ID NO: 355] | [SEQ ID NO: 356] |
| XD-14915 | CUCACGUUCAGCCGGCGUAUAC | AUACGCCGGCUGAACGUGAGAA |
| | [SEQ ID NO: 357] | [SEQ ID NO: 358] |
| XD-14916 | UCACGUUCAGCCGGCGUAUACC | UAUACGCCGGCUGAACGUGAGA |
| | [SEQ ID NO: 359] | [SEQ ID NO: 360] |
| XD-14917 | ACGUUCAGCCGGCGUAUACAAA | UGUAUACGCCGGCUGAACGUGA |
| | [SEQ ID NO: 361] | [SEQ ID NO: 362] |
| XD-14918 | CGUUCAGCCGGCGUAUACCAAC | UGGUAUACGCCGGCUGAACGUG |
| | [SEQ ID NO: 363] | [SEQ ID NO: 364] |
| XD-14919 | CCCACAUGGCCCACGUACCUCA | AGGUACGUGGGCCAUGUGGGGU |
| | [SEQ ID NO: 365] | [SEQ ID NO: 366] |
| XD-14920 | CCACAUGGCCCACGUACCUAAG | UAGGUACGUGGGCCAUGUGGGG |
| | [SEQ ID NO: 367] | [SEQ ID NO: 368] |
| XD-14921 | ACAUGGCCCACGUACCUCAAGC | UUGAGGUACGUGGGCCAUGUGG |
| | [SEQ ID NO: 369] | [SEQ ID NO: 370] |
| XD-14922 | CCAACAGCAGUUGUAAGGCUGC | AGCCUUACAACUGCUGUUGGUG |
| | [SEQ ID NO: 371] | [SEQ ID NO: 372] |
| XD-14923 | UCUUGUAACAUCCAAUAGGAAU | UCCUAUUGGAUGUUACAAGAAA |
| | [SEQ ID NO: 373] | [SEQ ID NO: 374] |
| XD-14924 | GACCGAGUAGAGGCAUUUAAGA | UUAAAUGCCUCUACUCGGUCCA |
| | [SEQ ID NO: 375] | [SEQ ID NO: 376] |
| XD-14925 | CGAGUAGAGGCAUUUAGGAACU | UUCCUAAAUGCCUCUACUCGGU |
| | [SEQ ID NO: 377] | [SEQ ID NO: 378] |
| XD-14926 | GGAACUUGGGGGCUAUUCCAUA | UGGAAUAGCCCCCAAGUUCCUA |
| | [SEQ ID NO: 379] | [SEQ ID NO: 380] |
| XD-14927 | ACUUGGGGGCUAUUCCAUAAUU | UUAUGGAAUAGCCCCCAAGUUC |
| | [SEQ ID NO: 381] | [SEQ ID NO: 382] |
| XD-14928 | UUGGGGGCUAUUCCAUAAUUCC | AAUUAUGGAAUAGCCCCCAAGU |
| | [SEQ ID NO: 383] | [SEQ ID NO: 384] |
| XD-14929 | GGGGGCUAUUCCAUAAUUCAAU | UGAAUUAUGGAAUAGCCCCCAA |
| | [SEQ ID NO: 385] | [SEQ ID NO: 386] |
| XD-14930 | GGGGCUAUUCCAUAAUUCCAUA | UGGAAUUAUGGAAUAGCCCCCA |
| | [SEQ ID NO: 387] | [SEQ ID NO: 388] |
| XD-14931 | CUCUGCUUGCCGAAACUGGAAG | UCCAGUUUCGGCAAGCAGAGCU |
| | [SEQ ID NO: 389] | [SEQ ID NO: 390] |
| XD-14932 | UUGCCGAAACUGGAAGUUAUUU | AUAACUUCCAGUUUCGGCAAGC |
| | [SEQ ID NO: 391] | [SEQ ID NO: 392] |
| XD-14933 | AUAACCCUUGAAAGUCAUGAAC | UCAUGACUUUCAAGGGUUAUUA |
| | [SEQ ID NO: 393] | [SEQ ID NO: 394] |
| XD-14934 | UGAACACAUCAGCUAGCAAAAG | UUUGCUAGCUGAUGUGUUCAUG |
| | [SEQ ID NO: 395] | [SEQ ID NO: 396] |
| XD-14935 | UGAUUCUUGCUGCUAUUACUGC | AGUAAUAGCAGCAAGAAUCACU |
| | [SEQ ID NO: 397] | [SEQ ID NO: 398] |
| XD-14936 | UGGAACGCCCUUUUACUAAACU | UUUAGUAAAAGGGCGUUCCAAG |
| | [SEQ ID NO: 399] | [SEQ ID NO: 400] |
| XD-14937 | GGAACGCCCUUUUACUAAAAUU | UUUUAGUAAAAGGGCGUUCCAA |
| | [SEQ ID NO: 401] | [SEQ ID NO: 402] |
| XD-14938 | GAACGCCCUUUUACUAAACUUG | AGUUUAGUAAAAGGGCGUUCCA |
| | [SEQ ID NO: 403] | [SEQ ID NO: 404] |
| XD-14939 | AACGCCCUUUUACUAAACUUGA | AAGUUUAGUAAAAGGGCGUUCC |
| | [SEQ ID NO: 405] | [SEQ ID NO: 406] |
| XD-14940 | AGUAAAUUCUUACCGUCAAACU | UUUGACGGUAAGAAUUUACUGA |
| | [SEQ ID NO: 407] | [SEQ ID NO: 408] |
| XD-14941 | GUAAAUUCUUACCGUCAAAAUG | UUUUGACGGUAAGAAUUUACUG |
| | [SEQ ID NO: 409] | [SEQ ID NO: 410] |
| XD-14942 | UCUUACCGUCAAACUGACGAAU | UCGUCAGUUUGACGGUAAGAAU |
| | [SEQ ID NO: 411] | [SEQ ID NO: 412] |
| XD-14943 | CUUACCGUCAAACUGACGGAUU | UCCGUCAGUUUGACGGUAAGAA |
| | [SEQ ID NO: 413] | [SEQ ID NO: 414] |
| XD-14944 | UUACCGUCAAACUGACGGAUUA | AUCCGUCAGUUUGACGGUAAGA |
| | [SEQ ID NO: 415] | [SEQ ID NO: 416] |
| XD-14945 | ACCGUCAAACUGACGGAUUAUU | UAAUCCGUCAGUUUGACGGUAA |
| | [SEQ ID NO: 417] | [SEQ ID NO: 418] |
| XD-14946 | CCGUCAAACUGACGGAUUAUUA | AUAAUCCGUCAGUUUGACGGUA |
| | [SEQ ID NO: 419] | [SEQ ID NO: 420] |
| XD-14947 | CGUCAAACUGACGGAUUAUUAU | AAUAAUCCGUCAGUUUGACGGU |
| | [SEQ ID NO: 421] | [SEQ ID NO: 422] |
| XD-14948 | GUCAAACUGACGGAUUAUUAUU | UAAUAAUCCGUCAGUUUGACGG |
| | [SEQ ID NO: 423] | [SEQ ID NO: 424] |
| XD-14949 | UCAAACUGACGGAUUAUUAUUU | AUAAUAAUCCGUCAGUUUGACG |
| | [SEQ ID NO: 425] | [SEQ ID NO: 426] |
| XD-14950 | ACUGACGGAUUAUUAUUUAUAA | AUAAAUAAUAAUCCGUCAGUUU |
| | [SEQ ID NO: 427] | [SEQ ID NO: 428] |
| XD-14951 | CUGACGGAUUAUUAUUUAUAAA | UAUAAAUAAUAAUCCGUCAGUU |
| | [SEQ ID NO: 429] | [SEQ ID NO: 430] |
| XD-14952 | AUGAGGUGAUCACUGUCUAAAG | UUAGACAGUGAUCACCUCAUCA |
| | [SEQ ID NO: 431] | [SEQ ID NO: 432] |
| XD-14953 | GAGGUGAUCACUGUCUACAAUG | UUGUAGACAGUGAUCACCUCAU |
| | [SEQ ID NO: 433] | [SEQ ID NO: 434] |
| XD-14954 | CUGUCUACAGUGGUUCAACUUU | AGUUGAACCACUGUAGACAGUG |
| | [SEQ ID NO: 435] | [SEQ ID NO: 436] |

In some embodiments, the isolated siRNA duplexes of the present disclosure, particularly when not delivered as an expression construct or within a vector, comprise at least one modified nucleotide, including a modified base, modified sugar, or modified backbone. siRNA having nucleotide modification(s) may have increased stability, increased specificity, reduced immunogenicity, or a combination thereof. Modified nucleotides may occur on either the guide strand, passenger strand, or both the guide strand and passenger strand.

Modified bases refer to nucleotide bases such as, for example, adenine, guanine, cytosine, thymine, uracil, xanthine, inosine, and queuosine that have been modified by the replacement or addition of one or more atoms or groups. Some examples of modifications on the nucleobase moieties include, but are not limited to, alkylated, halogenated, thiolated, aminated, amidated, or acetylated bases, individually or in combination. More specific examples include, for example, 5-propynyluridine, 5-propynylcytidine, 6-methyladenine, 6-methylguanine, N,N-dimethyladenine, 2-propyladenine, 2-propylguanine, 2-aminoadenine, 1-methylinosine, 3-methyluridine, 5-methylcytidine, 5-methyluridine and other nucleotides having a modification at the 5 position, 5-(2-amino)propyl uridine, 5-halocytidine, 5-halouridine, 4-acetylcytidine, 1-methyladenosine, 2-methyladenosine, 3-methylcytidine, 6-methyluridine, 2-methylguanosine, 7-methylguanosine, 2,2-dimethylguanosine, 5-methylaminoethyluridine, 5-methyloxyuridine, deazanucleotides such as 7-deaza-adenosine, 6-azouridine, 6-azocytidine, 6-azothymidine, 5-methyl-2-thiouridine, other thio bases such as 2-thiouridine and 4-thiouridine and 2-thiocytidine, dihydrouridine, pseudouridine, queuosine, archaeosine, naphthyl and substituted naphthyl groups, any O- and N-alkylated purines and pyrimidines such as N6-methyladenosine, 5-methylcarbonylmethyluridine, uridine 5-oxyacetic acid, pyridine-4-one, pyridine-2-one, phenyl and modified phenyl groups such as aminophenol or 2,4,6-trimethoxy benzene, modified cytosines that act as G-clamp nucleotides, 8-substituted adenines and guanines, 5-substituted uracils and thymines, azapyrimidines, carboxyhydroxyalkyl nucleotides, carboxyalkylaminoalkyl nucleotides, and alkylcarbonylalkylated nucleotides. Sugar modified nucleotides include, but are not limited to 2′-fluoro, 2′-amino and 2′-thio modified ribonucleotides, e.g., 2′-fluoro modified ribonucleotides.

Modified nucleotides may be modified on the sugar moiety, as well as be nucleotides having non-ribosyl sugars or analogs thereof. For example, the sugar moieties may be, or be based on, mannoses, arabinoses, glucopyranoses, galactopyranoses, 4′-thioribose, and other sugars, heterocycles, or carbocycles.

A normal “backbone,” as used herein, refers to the repeatingly alternating sugar-phosphate sequences in a DNA or RNA molecule. The deoxyribose/ribose sugars are joined at both the 3′-hydroxyl and 5′-hydroxyl groups to phosphate groups in ester links, also known as “phosphodiester” bonds or linkages. One or more, or all phosphodiester linkage(s) may be modified as phosphorothioate linkages, boranophosphate linkages, amide linkages, phosphorodithioate linkages, or triazole linkages.

In some embodiments, the inhibitory nucleic acid is a shRNA. In some embodiments, the shRNA is a stem-loop duplex molecule comprising a guide strand and passenger strand of a siRNA duplex as provided herein (e.g., siRNA duplexes of Tables 1 and 19), linked by a spacer sequence, i.e., loop. In some embodiments, loop sequence is 4, 5, 6, 7, 8, 9, 10, 11, 12, 13, 14, 15, 16, 17, 18, 19, 20, 21, 22, 23, 24, 25 nucleotides in length or 4-25, 4-24, 4-23, 4-22, 4-21, 4-20, 4-19, 4-18, 4-17, 4-16, 4-15, 4-14, 4-11, 4-10, 4-9, 4-8, 4-7, 4-6, 5-25, 5-24, 5-23, 5-22, 5-21, 5-20, 5-19, 5-18, 5-17, 5-16, 5-15, 5-14, 5-13, 5-12, 5-11, 5-10, 5-9, 5-8, 5-7, 6-25, 6-24, 6-23, 6-22, 6-21, 6-20, 6-19, 6-18, 6-17, 6-16, 6-15, 6-14, 6-13, 6-12, 6-11, 6-10, 6-9, 6-8, 7-25, 7-24, 7-23, 7-22, 7-21, 7-20, 7-19, 7-18, 7-17, 7-16, 7-15, 7-14, 7-13, 7-12, 7-11, 7-10, 7-9, 8-25, 8-24, 8-23, 8-22, 8-21, 8-20, 8-19, 8-18, 8-11, 8-10, 9-25, 9-24, 9-23, 9-22, 9-21, 9-20, 9-19, 9-18, 9-17, 9-16, 9-15, 9-14, 9-13, 9-12, 9-11, 10-25, 10-24, 10-23, 10-22, 10-21, 10-20, 10-19, 10-18, 10-17, 10-16, 10-15, 10-14, 10-13, 10-12, 11-25, 11-24, 11-23, 11-22, 11-20, 11-19, 11-18, 11-17, 11-16, 11-15, 11-14, 11-13, 12-25, 12-24, 12-23, 12-22, 12-21, 12-20, 12-19, 12-18, 12-17, 12-16, 12-15, or 12-14 nucleotides in length.

In some embodiments, the inhibitory nucleic acid is an isolated miRNA. A miRNA may be a pri-mRNA, a pre-mRNA, mature miRNA, or artificial miRNA. In some embodiments, a miRNA is comprised of a guide strand and passenger strand. In some embodiments, the guide strand and passenger strand are within the same nucleic acid strand, where the guide strand and passenger strand hybridize together to form a self-annealing duplex structure. MiRNA is initially transcribed as a pri-mRNA, which is processed by nuclear nuclease (e.g., Drosha-DGCR8 complex) into pre-mRNA. A pri-mRNA is a single-stranded molecule having a stem-loop structure. In some embodiments, the pri-miRNA is about 100, 150, 200, 300, 400, 500, 600, 700, 800, 900, 1000, 1200, 1300, 1400, 1500, 1600, 1700, 1800, 1900, 2000, 2500, 3000 or more nucleotides in length or about 100-3000, 100-2500, 100-2000, 100-1900, 100-1800, 100-1700, 100-1600, 100-1500, 100-1400, 100-1300, 100-1200, 100-1100, 100-1000, 100-900, 100-800, 100-700, 100-600, 100-500, 100-400, 100-300, 100-200, 100-150, 150-3000, 150-2500, 150-2000, 150-1900, 150-1800, 150-1700, 150-1600, 150-1500, 150-1400, 150-1300, 150-1200, 150-1100, 150-1000, 150-900, 150-800, 150-700, 150-600, 150-500, 150-400, 150-300, 150-200, 200-3000, 200-2500, 200-2000, 200-1900, 200-1800, 200-1700, 200-1600, 200-1500, 200-1400, 200-1300, 200-1200, 200-1100, 200-1000, 200-900, 200-800, 200-700, 200-600, 200-500, 200-400, 200-300, 300-3000, 300-2500, 300-2000, 300-1900, 300-1800, 300-1700, 300-1600, 300-1500, 300-1400, 300-1300, 300-1200, 300-1100, 300-1000, 300-900, 300-800, 300-700, 300-600, 300-500, 300-400, 400-3000, 400-2500, 400-2000, 400-1900, 400-1800, 400-1700, 400-1600, 400-1500, 400-1400, 400-1300, 400-1200, 400-1100, 400-1000, 400-900, 400-800, 400-700, 400-600, 400-500, 500-3000, 500-2500, 500-2000, 500-1900, 500-1800, 500-1700, 500-1600, 500-1500, 500-1400, 500-1300, 500-1200, 500-1100, 500-1000, 500-900, 500-800, 500-700, or 500-600 nucleotides in length.

Pre-miRNA is also a single-stranded molecule having a stem-loop structure. In some embodiments, the pre-miRNA is about 40, 50, 60, 70, 80, 90, 100, 200, 300, 400, or 500 nucleotides in length, or about 40-500, 40-400, 40-300, 40-200, 40-100, 40-90, 40-80, 40-70, 40-60, 40-50, 50-500, 50-400, 50-300, 50-200, 50-100, 50-90, 50-80, 50-70, 60-500, 60-400, 60-300, 60-200, 60-100, 60-90, 60-80, 70-500, 70-400, 70-300, 70-200, 70-100, 70-90, 80-500, 80-400, 80-300, 80-200, 80-100, 90-500, 90-400, 90-300, 90-200, 100-500, 100-400, 100-300, 100-200, 200-500, 200-400, 200-300, 300-500, 300-400, or 400-500 nucleotides in length.

The pre-miRNA is transported from the nucleus to the cytoplasm by exportin-5 and further processed by Dicer to produce a mature, double-stranded miRNA duplex comprising a guide strand and a passenger strand. The mature miRNA duplex is then incorporated into the RNA inducing silencing complex (RISC), mediated by TRBP (HIV transactivating response RNA-binding protein). The passenger strand is generally released and cleaved, while the guide strand remains in RISC and binds to the target mRNA and mediates silencing. In some embodiments, a mature miRNA refers to the guide strand of a mature miRNA duplex. In some embodiments, a mature miRNA is about 19, 20, 21, 22, 23, 24, 25, 26, 27, 28, 29, or 30 nucleotides in length, or ranges from about 19-30 nucleotides, 19-29 nucleotides, 19-28 nucleotides, 19-27 nucleotides, 19-26 nucleotides, 19-25 nucleotides, 19-24 nucleotides, 19-23 nucleotides, 19-21 nucleotides, 20-30 nucleotides, 20-29 nucleotides, 20-28 nucleotides, 20-27 nucleotides, 20-26 nucleotides, 20-25 nucleotides, 20-24 nucleotides, 20-23 nucleotides, 20-22 nucleotides, 21-30 nucleotides, 21-29 nucleotides, 21-28 nucleotides, 21-27 nucleotides, 21-26 nucleotides, 21-25 nucleotides, 21-24 nucleotides, 21-23 nucleotides, 22-30 nucleotides, 22-29 nucleotides, 22-28 nucleotides, 22-27 nucleotides, 22-26 nucleotides, 22-25 nucleotides, 22-24 nucleotides, 23-30 nucleotides, 23-29 nucleotides, 23-28 nucleotides, 23-27 nucleotides, 23-26 nucleotides, 23-25 nucleotides, 24-30 nucleotides, 24-29 nucleotides, 24-28 nucleotides, 24-27 nucleotides, 24-26 nucleotides, 25-30 nucleotides, 25-29 nucleotides, 25-28 nucleotides, 25-27 nucleotides, 26-30 nucleotides, 26-29 nucleotides, 26-28 nucleotides, 27-30 nucleotides, 27-29 nucleotides, or 28-30 nucleotides in length.

Artificial miRNA refers to an endogenous, modified or synthetic pri-mRNA or pre-mRNA scaffold or backbone capable of producing a functional mature miRNA, where the guide strand sequence and passenger strand sequence of the miRNA duplex within the stem region have been replaced with a guide strand sequence and passenger strand sequence of interest that directs silencing of the target mRNA of interest. Artificial miRNA design is described in Eamens et al. (2014) Methods Mol Biol. 1062:211-24 (incorporated by reference in its entirety). Synthetic miRNA backbones are described in U.S. Patent Publication 2008/0313773 (incorporated by reference in its entirety). In some embodiments, the artificial miRNA is about 100-200 nucleotides, 100-175 nucleotides 100-150 nucleotides, 125-200 nucleotides 125-175 nucleotides, or 125-150 nucleotides in length. In some embodiments, the artificial miRNA is about 100 nucleotides, about 120 nucleotides, about 130 nucleotides, about 140 nucleotides, about 150 nucleotides, about 160 nucleotides, about 170 nucleotides, about 180 nucleotides, about 190 nucleotides, or about 200 nucleotides in length.

In some embodiments, the miRNA is a pri-miRNA, a pre-mRNA, an artificial miRNA, or a mature miRNA comprising a guide strand sequence comprising or consisting of the nucleic acid sequence set forth in any one of the guide sequences of Tables 1, 3, 9, 11, 12, 13, 19, 23, 24, and 25, e.g., any one of SEQ ID NOS: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 1176-1288, 1811-1827, 2015, 2065, 2083, 2152, 2203, and 2209.

In some embodiments, the miRNA is a pri-miRNA, a pre-mRNA, an artificial miRNA, or a mature miRNA comprising a guide strand sequence comprising or consisting of the nucleic acid sequence set forth in any one of the guide sequences of Tables 1, 3, 9, 11, 12, 13, 19, 23, 24, and 25, e.g., any one of SEQ ID NOS: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 1176-1288, 1811-1827, 2015, 2065, 2083, 2152, 2203, and 2209, with at least 1, 2, 3, 4, or 5 mismatches to the target ATXN2 mRNA sequence.

In some embodiments, the miRNA is a pri-miRNA, a pre-mRNA, an artificial miRNA, or a mature miRNA comprising a guide strand sequence comprising or consisting of a sequence that is at least 60%, 70%, 75%, 80%, 85%, 90%, 95%, 97%, 99%, or 100% identical to any one of the guide sequences of Tables 1, 3, 9, 11, 12, 13, 19, 23, 24, and 25, e.g., any one of SEQ ID NOS: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 1176-1288, 1811-1827, 2015, 2065, 2083, 2152, 2203, and 2209.

In some embodiments, the miRNA is a pri-miRNA, a pre-mRNA, an artificial miRNA, or a mature miRNA comprising a guide strand sequence comprising or consisting of at least 15, 16, 17, 18, 19, 20, 21, or 22 contiguous nucleotides of a sequence of any one of the guide sequences of Tables 1, 3, 9, 11, 12, 13, 19, 23, 24, and 25, e.g., any one of SEQ ID NOS: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 1176-1288, 1811-1827, 2015, 2065, 2083, 2152, 2203, and 2209, preferably wherein the guide strand sequence retains positions 2-7 (“seed sequence”) of the selected SEQ ID NO.

In some embodiments, the miRNA is a pri-miRNA, a pre-mRNA, an artificial miRNA, or a mature miRNA comprising a guide strand sequence comprising or consisting of a sequence of any one of the guide sequences of Tables 1, 3, 9, 11, 12, 13, 19, 23, 24, and 25, e.g., any one of SEQ ID NOS: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 1176-1288, 1811-1827, 2015, 2065, 2083, 2152, 2203, and 2209, wherein 1, 2, 3, or 4 nucleotides at positions 19-22 differ from the selected SEQ ID NO (variant nucleotide(s)) such that the guide strand sequence is no longer complementary to the ATXN2 target sequence at the variant nucleotide(s).

In some embodiments, the miRNA is a pri-miRNA, a pre-mRNA, an artificial miRNA, or a mature miRNA comprising a guide strand sequence of Table 12. In some embodiments, the miRNA is a pri-miRNA, a pre-mRNA, an artificial miRNA, or a mature miRNA comprising a guide strand sequence comprising or consisting of the nucleic acid sequence set forth in any one of SEQ ID NOS: 12, 14, 40, 60, 100, 104, 108, 112, 124, 126, 128, 166, 198, 220, 242, 302, 306, 308, 330, 336, and 362. In some embodiments, the miRNA is a pri-miRNA, a pre-mRNA, an artificial miRNA, or a mature miRNA comprising a guide strand sequence comprising or consisting of the nucleic acid sequence set forth in any one of SEQ ID NOS: 12, 14, 40, 60, 100, 104, 108, 112, 124, 126, 128, 166, 198, 220, 242, 302, 306, 308, 330, 336, and 362, with at least 1, 2, 3, 4, or 5 mismatches to the target ATXN2 mRNA sequence. In some embodiments, the miRNA is a pri-miRNA, a pre-mRNA, an artificial miRNA, or a mature miRNA comprising a guide strand sequence comprising or consisting of a nucleic acid sequence that is at least 60%, 70%, 75%, 80%, 85%, 90%, 95%, 97%, 99%, or 100% identical to any one of SEQ ID NOS: 12, 14, 40, 60, 100, 104, 108, 112, 124, 126, 128, 166, 198, 220, 242, 302, 306, 308, 330, 336, and 362. In some embodiments, the miRNA is a pri-miRNA, a pre-mRNA, an artificial miRNA, or a mature miRNA comprising a guide strand sequence comprising or consisting of at least 15, 16, 17, 18, 19, 20, 21, or 22 contiguous nucleotides of a sequence of any one of SEQ ID NOS: 12, 14, 40, 60, 100, 104, 108, 112, 124, 126, 128, 166, 198, 220, 242, 302, 306, 308, 330, 336, and 362, preferably wherein the guide strand sequence retains positions 2-7 (“seed sequence”) of the selected SEQ ID NO. In some embodiments, the miRNA is a pri-miRNA, a pre-mRNA, an artificial miRNA, or a mature miRNA comprising a guide strand sequence comprising or consisting of a sequence of any one of SEQ ID NOS: 12, 14, 40, 60, 100, 104, 108, 112, 124, 126, 128, 166, 198, 220, 242, 302, 306, 308, 330, 336, and 362, wherein 1, 2, 3, or 4 nucleotides at positions 19-22 differ from the selected SEQ ID NO (variant nucleotide(s)) such that the guide strand sequence is no longer complementary to the ATXN2 target sequence at the variant nucleotide(s).

In some embodiments, the miRNA is a pri-miRNA, a pre-mRNA, an artificial miRNA, or a mature miRNA comprising a guide strand sequence of Table 13. In some embodiments, the miRNA is a pri-miRNA, a pre-mRNA, an artificial miRNA, or a mature miRNA comprising a guide strand sequence comprising or consisting of the nucleic acid sequence set forth in any one of SEQ ID NOS: 14, 40, 100, 108, 112, 128, 166, 198, 242, 308, 336, and 362. In some embodiments, the miRNA is a pri-miRNA, a pre-mRNA, an artificial miRNA, or a mature miRNA comprising a guide strand sequence comprising or consisting of the nucleic acid sequence set forth in any one of SEQ ID NOS: 14, 40, 100, 108, 112, 128, 166, 198, 242, 308, 336, and 362, with at least 1, 2, 3, 4, or 5 mismatches to the target ATXN2 mRNA sequence. In some embodiments, the miRNA is a pri-miRNA, a pre-mRNA, an artificial miRNA, or a mature miRNA comprising a guide strand sequence comprising or consisting of a nucleic acid sequence that is at least 60%, 70%, 75%, 80%, 85%, 90%, 95%, 97%, 99%, or 100% identical to any one of SEQ ID NOS: 14, 40, 100, 108, 112, 128, 166, 198, 242, 308, 336, and 362. In some embodiments, the miRNA is a pri-miRNA, a pre-mRNA, an artificial miRNA, or a mature miRNA comprising a guide strand sequence comprising or consisting of at least 15, 16, 17, 18, 19, 20, 21, or 22 contiguous nucleotides of a sequence of any one of SEQ ID NOS: 14, 40, 100, 108, 112, 128, 166, 198, 242, 308, 336, and 362, preferably wherein the guide strand sequence retains positions 2-7 (“seed sequence”) of the selected SEQ ID NO. In some embodiments, the miRNA is a pri-miRNA, a pre-mRNA, an artificial miRNA, or a mature miRNA comprising a guide strand sequence comprising or consisting of a sequence of any one of SEQ ID NOS: 14, 40, 100, 108, 112, 128, 166, 198, 242, 308, 336, and 362, wherein 1, 2, 3, or 4 nucleotides at positions 19-22 differ from the selected SEQ ID NO (variant nucleotide(s)) such that the guide strand sequence is no longer complementary to the ATXN2 target sequence at the variant nucleotide(s).

In some embodiments, the miRNA is a pri-miRNA, a pre-mRNA, an artificial miRNA, or a mature miRNA comprising a guide strand sequence of Table 19. In some embodiments, the miRNA is a pri-miRNA, a pre-mRNA, an artificial miRNA, or a mature miRNA comprising a guide strand sequence comprising or consisting of the nucleic acid sequence set forth in any one of SEQ ID NOS:1176-1288, 40, 108, and 166. In some embodiments, the miRNA is a pri-miRNA, a pre-mRNA, an artificial miRNA, or a mature miRNA comprising a guide strand sequence comprising or consisting of the nucleic acid sequence set forth in any one of SEQ ID NOS:1176-1288, 40, 108, and 166, with at least 1, 2, 3, 4, or 5 mismatches to the target ATXN2 mRNA sequence. In some embodiments, the miRNA is a pri-miRNA, a pre-mRNA, an artificial miRNA, or a mature miRNA comprising a guide strand sequence comprising or consisting of a nucleic acid sequence that is at least 60%, 70%, 75%, 80%, 85%, 90%, 95%, 97%, 99%, or 100% identical to any one of SEQ ID NOS:1176-1288, 40, 108, and 166. In some embodiments, the miRNA is a pri-miRNA, a pre-mRNA, an artificial miRNA, or a mature miRNA comprising a guide strand sequence comprising or consisting of at least 15, 16, 17, 18, 19, 20, 21, or 22 contiguous nucleotides of a sequence of any one of SEQ ID NOS: 1176-1288, 40, 108, and 166, preferably wherein the guide strand sequence retains positions 2-7 (“seed sequence”) of the selected SEQ ID NO. In some embodiments, the miRNA is a pri-miRNA, a pre-mRNA, an artificial miRNA, or a mature miRNA comprising a guide strand sequence comprising or consisting of a sequence of any one of SEQ ID NOS:1176-1288, 40, 108, and 166, wherein 1, 2, 3, or 4 nucleotides at positions 19-22 differ from the selected SEQ ID NO (variant nucleotide(s)) such that the guide strand sequence is no longer complementary to the ATXN2 target sequence at the variant nucleotide(s).

In some embodiments, the miRNA is a pri-miRNA, a pre-mRNA, an artificial miRNA, or a mature miRNA comprising a guide strand sequence of Table 23. In some embodiments, the miRNA is a pri-miRNA, a pre-mRNA, an artificial miRNA, or a mature miRNA comprising a guide strand sequence comprising or consisting of the nucleic acid sequence set forth in any one of SEQ ID NOS:1908-2007. In some embodiments, the miRNA is a pri-miRNA, a pre-mRNA, an artificial miRNA, or a mature miRNA comprising a guide strand sequence comprising or consisting of the nucleic acid sequence set forth in any one of SEQ ID NOS:1908-2007, with at least 1, 2, 3, 4, or 5 mismatches to the target ATXN2 mRNA sequence. In some embodiments, the miRNA is a pri-miRNA, a pre-mRNA, an artificial miRNA, or a mature miRNA comprising a guide strand sequence comprising or consisting of a nucleic acid sequence that is at least 60%, 70%, 75%, 80%, 85%, 90%, 95%, 97%, 99%, or 100% identical to any one of SEQ ID NOS:1908-2007. In some embodiments, the miRNA is a pri-miRNA, a pre-mRNA, an artificial miRNA, or a mature miRNA comprising a guide strand sequence comprising or consisting of at least 15, 16, 17, 18, 19, 20, 21, or 22 contiguous nucleotides of a sequence of any one of SEQ ID NOS:1908-2007, preferably wherein the guide strand sequence retains positions 2-7 (“seed sequence”) of the selected SEQ ID NO. In some embodiments, the miRNA is a pri-miRNA, a pre-mRNA, an artificial miRNA, or a mature miRNA comprising a guide strand sequence comprising or consisting of a sequence of any one of SEQ ID NOS:1908-2007, wherein 1, 2, 3, or 4 nucleotides at positions 19-22 differ from the selected SEQ ID NO (variant nucleotide(s)) such that the guide strand sequence is no longer complementary to the ATXN2 target sequence at the variant nucleotide(s).

In some embodiments, the miRNA is a pri-miRNA, a pre-mRNA, an artificial miRNA, or a mature miRNA comprising a guide strand sequence of Table 24. In some embodiments, the miRNA is a pri-miRNA, a pre-mRNA, an artificial miRNA, or a mature miRNA comprising a guide strand sequence comprising or consisting of the nucleic acid sequence set forth in any one of SEQ ID NOS:100, 112, 166, 202, 246, 306, 308, 314, 1180, 1185, 1196, 1200, 1211, 1213, 1215, 1216, 1224, 1811-1822, 1824-1827, 2015, 2065, 2083, 2152, 2203, and 2209. In some embodiments, the miRNA is a pri-miRNA, a pre-mRNA, an artificial miRNA, or a mature miRNA comprising a guide strand sequence comprising or consisting of the nucleic acid sequence set forth in any one of SEQ ID NOS:100, 112, 166, 202, 246, 306, 308, 314, 1180, 1185, 1196, 1200, 1211, 1213, 1215, 1216, 1224, 1811-1822, 1824-1827, 2015, 2065, 2083, 2152, 2203, and 2209, with at least 1, 2, 3, 4, or 5 mismatches to the target ATXN2 mRNA sequence. In some embodiments, the miRNA is a pri-miRNA, a pre-mRNA, an artificial miRNA, or a mature miRNA comprising a guide strand sequence comprising or consisting of a nucleic acid sequence that is at least 60%, 70%, 75%, 80%, 85%, 90%, 95%, 97%, 99%, or 100% identical to any one of SEQ ID NOS:100, 112, 166, 202, 246, 306, 308, 314, 1180, 1185, 1196, 1200, 1211, 1213, 1215, 1216, 1224, 1811-1822, 1824-1827, 2015, 2065, 2083, 2152, 2203, and 2209. In some embodiments, the miRNA is a pri-miRNA, a pre-mRNA, an artificial miRNA, or a mature miRNA comprising a guide strand sequence comprising or consisting of at least 15, 16, 17, 18, 19, 20, 21, or 22 contiguous nucleotides of a sequence of any one of SEQ ID NOS:100, 112, 166, 202, 246, 306, 308, 314, 1180, 1185, 1196, 1200, 1211, 1213, 1215, 1216, 1224, 1811-1822, 1824-1827, 2015, 2065, 2083, 2152, 2203, and 2209, preferably wherein the guide strand sequence retains positions 2-7 (“seed sequence”) of the selected SEQ ID NO. In some embodiments, the miRNA is a pri-miRNA, a pre-mRNA, an artificial miRNA, or a mature miRNA comprising a guide strand sequence comprising or consisting of a sequence of any one of SEQ ID NOS:100, 112, 166, 202, 246, 306, 308, 314, 1180, 1185, 1196, 1200, 1211, 1213, 1215, 1216, 1224, 1811-1822, 1824-1827, 2015, 2065, 2083, 2152, 2203, and 2209, wherein 1, 2, 3, or 4 nucleotides at positions 19-22 differ from the selected SEQ ID NO (variant nucleotide(s)) such that the guide strand sequence is no longer complementary to the ATXN2 target sequence at the variant nucleotide(s).

In some embodiments, the miRNA is a pri-miRNA, a pre-mRNA, an artificial miRNA, or a mature miRNA comprising a guide strand sequence of Table 25. In some embodiments, the miRNA is a pri-miRNA, a pre-mRNA, an artificial miRNA, or a mature miRNA comprising a guide strand sequence comprising or consisting of the nucleic acid sequence set forth in any one of SEQ ID NOS:1185, 1816, 1213, 1819, 2083, 1215, 1216, 1811, and 314. In some embodiments, the miRNA is a pri-miRNA, a pre-mRNA, an artificial miRNA, or a mature miRNA comprising a guide strand sequence comprising or consisting of the nucleic acid sequence set forth in any one of SEQ ID NOS:1185, 1816, 1213, 1819, 2083, 1215, 1216, 1811, and 314, with at least 1, 2, 3, 4, or 5 mismatches to the target ATXN2 mRNA sequence. In some embodiments, the miRNA is a pri-miRNA, a pre-mRNA, an artificial miRNA, or a mature miRNA comprising a guide strand sequence comprising or consisting of a nucleic acid sequence that is at least 60%, 70%, 75%, 80%, 85%, 90%, 95%, 97%, 99%, or 100% identical to any one of SEQ ID NOS:1185, 1816, 1213, 1819, 2083, 1215, 1216, 1811, and 314. In some embodiments, the miRNA is a pri-miRNA, a pre-mRNA, an artificial miRNA, or a mature miRNA comprising a guide strand sequence comprising or consisting of at least 15, 16, 17, 18, 19, 20, 21, or 22 contiguous nucleotides of a sequence of any one of SEQ ID NOS:1185, 1816, 1213, 1819, 2083, 1215, 1216, 1811, and 314, preferably wherein the guide strand sequence retains positions 2-7 (“seed sequence”) of the selected SEQ ID NO. In some embodiments, the miRNA is a pri-miRNA, a pre-mRNA, an artificial miRNA, or a mature miRNA comprising a guide strand sequence comprising or consisting of a sequence of any one of SEQ ID NOS:1185, 1816, 1213, 1819, 2083, 1215, 1216, 1811, and 314, wherein 1, 2, 3, or 4 nucleotides at positions 19-22 differ from the selected SEQ ID NO (variant nucleotide(s)) such that the guide strand sequence is no longer complementary to the ATXN2 target sequence at the variant nucleotide(s).

In some embodiments, the miRNA is a pri-miRNA, a pre-mRNA, an artificial miRNA, or a mature miRNA comprising a guide strand sequence comprising or consisting of the nucleic acid sequence set forth in any one of SEQ ID NOS:1185, 1816, 1213, and 1811. In some embodiments, the miRNA is a pri-miRNA, a pre-mRNA, an artificial miRNA, or a mature miRNA comprising a guide strand sequence comprising or consisting of the nucleic acid sequence set forth in any one of SEQ ID NOS:1185, 1816, 1213, and 1811, with at least 1, 2, 3, 4, or 5 mismatches to the target ATXN2 mRNA sequence. In some embodiments, the miRNA is a pri-miRNA, a pre-mRNA, an artificial miRNA, or a mature miRNA comprising a guide strand sequence comprising or consisting of a nucleic acid sequence that is at least 60%, 70%, 75%, 80%, 85%, 90%, 95%, 97%, 99%, or 100% identical to any one of SEQ ID NOS:1185, 1816, 1213, and 1811. In some embodiments, the miRNA is a pri-miRNA, a pre-mRNA, an artificial miRNA, or a mature miRNA comprising a guide strand sequence comprising or consisting of at least 15, 16, 17, 18, 19, 20, 21, or 22 contiguous nucleotides of a sequence of any one of SEQ ID NOS:1185, 1816, 1213, and 1811, preferably wherein the guide strand sequence retains positions 2-7 (“seed sequence”) of the selected SEQ ID NO. In some embodiments, the miRNA is a pri-miRNA, a pre-mRNA, an artificial miRNA, or a mature miRNA comprising a guide strand sequence comprising or consisting of a sequence of any one of SEQ ID NOS:1185, 1816, 1213, and 1811, wherein 1, 2, 3, or 4 nucleotides at positions 19-22 differ from the selected SEQ ID NO (variant nucleotide(s)) such that the guide strand sequence is no longer complementary to the ATXN2 target sequence at the variant nucleotide(s).

In some embodiments, an artificial miRNA comprises a guide strand sequence according to any of the embodiments described herein, contained within a miR backbone sequence. In some embodiments, the guide strand sequence and passenger strand sequence of the artificial miRNA are contained with a miRNA backbone sequence. In some embodiments, the miRNA backbone sequence is a miR-155 backbone sequence, a miR-155E backbone sequence, a miR-155M backbone sequence, a miR1-1 backbone sequence, a miR-1-1_M backbone sequence, a miR-100 backbone sequence, a miR-100_M backbone sequence, a miR-190A backbone sequence, a miR-124 backbone sequence, a miR-124_M backbone sequence, a miR-16-2 backbone sequence, a miR-132 backbone sequence, a miR-9 backbone sequence, a miR-138-2 backbone sequence, a miR-122 backbone sequence, a miR-122_M backbone sequence, a miR-130a backbone sequence, miR-128 backbone sequence, a miR-144 backbone sequence, a miR-451a backbone sequence, or a miR-223 backbone sequence.

In some embodiments, the miRNA backbone sequence is a miR-155E backbone sequence, a miR-155M backbone sequence, a miR1-1 backbone sequence, a miR-1-1_M backbone sequence, a miR-100 backbone sequence, a miR-100_M backbone sequence, a miR-190a backbone sequence, a miR-190a_M backbone sequence, a miR-124 backbone sequence, a miR-124_M backbone sequence, a miR-132 backbone sequence, a miR-138-2 backbone sequence, a miR-122 backbone sequence, a miR-122_M backbone sequence, a miR-130a backbone sequence, a miR-16-2 backbone sequence, a miR-128 backbone sequence, a miR-144 backbone sequence, a miR-451a backbone sequence, or a miR-223 backbone sequence.

In some embodiments, the miRNA backbone sequence is a miR1-1 backbone sequence, a miR-1-1_M backbone sequence, a miR-100 backbone sequence, a miR-100_M backbone sequence, a miR-122 backbone sequence, a miR-122_M backbone sequence, a miR-124 backbone sequence, a miR-130a backbone sequence, a miR-132 backbone sequence, a miR-138-2 backbone sequence, a miR-144 backbone sequence, a miR-155E backbone sequence, a miR-155M backbone sequence, a miR-190a_M backbone sequence, or a miR-190a_M backbone sequence.

In some embodiments, the miRNA backbone sequence is a miR-100 backbone sequence or miR-100_M backbone sequence.

Table 2 provides examples of DNA sequences representing segments in miR-1-1, miR-100, miR-122, miR-124, miR-128, miR-130a, miR-155E, miR-155-M, and miR-138-2 backbones. Table 21 provides examples of DNA sequences representing segments in miR-1-1, miR-1-1_M, miR-100, miR-100_M, miR-122, miR-122_M, miR-124, miR-124 M, miR-128, miR-130a, miR-155E, miR-155M, miR-138-2, miR-144, miR-190a, miR-190a_M, miR-132, miR-451a, miR-223, and miR-16-2 backbones. It is understood that RNA sequences of the miR backbone segments in Tables 2 and 21 may be obtained by converting the “T” nucleotides in the sequences of Tables 2 and 21 to “U” nucleotides. Artificial miRNAs may be designed to insert desired guide and passenger sequences of the present disclosure into the miRNA backbones as defined in Table 2 or 21, and optionally wherein the passenger sequence is designed according to the rules in Table 8. For example, an artificial miRNA with miR-100 backbone in DNA format (e.g., for insertion into a transfer plasmid) may be designed according to Table 21 comprising from 5′ to 3′:5′ miR context (flanking) sequence of SEQ ID NO:1529; 5′ basal stem sequence of SEQ ID NO:1530; desired guide sequence; loop sequence of SEQ ID NO:1531; desired passenger sequence designed according to the rules in Table 8; 3′ basal stem sequence of SEQ ID NO:1532; and 3′ miR context (flanking) sequence of SEQ ID NO:1533.

TABLE 2
Annotation of miR Backbone Sequences

| | 5′ miR context | | | | | | 3′ miR context |
|---|---|---|---|---|---|---|---|
| | (flanking | | | | | 3′ basal | (flanking |
| miR | segment) | 5′ basal stem | 5p | Terminal loop | 3p | stem | segment) |
| miR-1-1 | catgcagactgcct | TGGG | pas- | TATGGACCTGCTA | guide | CTCA | ggccgggacctctc |
| | gct | [SEQ ID NO: | sen- | AGCTA | | [SEQ ID NO: | tcgccgcactgagg |
| | [SEQ ID NO: | 492] | ger | [SEQ ID NO: | | 494] | ggcactccacacca |
| | 491] | | | 493] | | | cgggggccg |
| | | | | | | | [SEQ ID NO: |
| | | | | | | | 495] |
| miR-100 | CCCAAAAGAGAGAA | CCTGTTGCCACA | guide | GTATTAGTCCG | pas- | TGTGTCTGTTA | CAATCTCACGGACC |
| | GATATTGAGG | [SEQ ID NO: | | [SEQ ID NO: | sen- | GG | TGGGGCTTTGCTTA |
| | [SEQ ID NO: | 497] | | 498] | ger | [SEQ ID NO: | TATGCC |
| | 496] | | | | | 499] | [SEQ ID NO: |
| | | | | | | | 500] |
| miR-122 | ggctacagagttt | CCTTAGCAGAGCT | guide | TGTCTAAACTAT | pas- | TAGCTACTGCT | aatccttccctcga |
| | [SEQ ID NO: | G | | [SEQ ID NO: | sen- | AGGC | taaatgtcttggca |
| | 501] | [SEQ ID NO: | | 503] | ger | [SEQ ID NO: | tcgtttgctttg |
| | | 502] | | | | 504] | [SEQ ID NO: |
| | | | | | | | 505] |
| miR-124 | TTCCTTCCTCAGGA | AGGCCTCTCTC | pas- | ATTTAAATGTCCA | guide | GAATGGGGCTC | GCTGAGCACCGTGG |
| | GAA | [SEQ ID NO: | sen- | TACAAT | | [SEQ ID NO: | GTCGGCGAGGGCCC |
| | [SEQ ID NO: | 507] | ger | [SEQ ID NO: | | 509] | GCCAagga |
| | 506] | | | 508] | | | [SEQ ID NO: |
| | | | | | | | 510] |
| miR-128 | ATTTtgcaataatt | TGAGCTGTTGGA | pas- | GAGGTTTACATTT | guide | TTCAGCTGCTT | ctggcttcttttta |
| | ggccttgttcc | [SEQ ID NO: | sen- | C | | C | ctcaggtttccact |
| | [SEQ ID NO: | 512] | ger | [SEQ ID NO: | | [SEQ ID NO: | gct |
| | 511] | | | 513] | | 514] | [SEQ ID NO: |
| | | | | | | | 515] |
| miR-130a | gcagggccggcatg | TGCTGCTGGCCA | pas- | CTGTCTGCACCTG | guide | TGGCCGTGTAG | Ctacccagcgctgg |
| | cctc | [SEQ ID NO: | sen- | TCACTAG | | TG | ctgcctcctcagca |
| | [SEQ ID NO: | 517] | ger | [SEQ ID NO: | | [SEQ ID NO: | ttg |
| | 516] | | | 518] | | 519] | [SEQ ID NO: |
| | | | | | | | 520] |
| miR-155E | CTGGAGGCTTGCTT | GGGCTGTATGCTG | guide | TTTTGGCCTCTGA | pas- | CAGGACAAGGC | TTTATCAGCACTCA |
| | T | [SEQ ID NO: | | CTGA | sen- | CC | CATGGAACAAATGG |
| | [SEQ ID NO: | 522] | | [SEQ ID NO: | ger | [SEQ ID NO: | CCACCGTG |
| | 521] | | | 523] | | 524] | [SEQ ID NO: |
| | | | | | | | 525] |
| miR-155- | CCTGGAGGCTTGCT | AGGCTGTATGCTG | guide | TTTTGGCCACTGA | pas- | CAGGACACAAG | TGTTACTAGCACTC |
| M | GA | [SEQ ID NO: | | CTGA | sen- | GCC | ACATGGAACAAATG |
| | [SEQ ID NO: | 527] | | [SEQ ID NO: | ger | [SEQ ID NO: | GCCACC |
| | 526] | | | 528] | | 529] | [SEQ ID NO: |
| | | | | | | | 530] |
| miR-138- | gccggcggagttct | CGTTGCTGC | guide | GACGAGCAGCGCA | pas- | GTTGCATCA | tacccatcctctcc |
| 2 | ggtat | [SEQ ID NO: | | TCCTCTTACCC | sen- | [SEQ ID NO: | aggcgagcctcgtg |
| | [SEQ ID NO: | 532] | | [SEQ ID NO: | ger | 534] | ggacc |
| | 531] | | | 533] | | | [SEQ ID NO: |
| | | | | | | | 535] |

In some embodiments, the terminal loop, stem, 5′ flanking segment, 3′ flanking segment, or any combination thereof of the miR-155 backbone sequence, miR1-1 backbone sequence, miR-100 backbone sequence, miR-190A backbone sequence, miR-124 backbone sequence, miR-16-2 backbone sequence, miR-132 backbone sequence, miR-9 backbone sequence, miR-138-2 backbone sequence, miR-122 backbone sequence, miR-130a backbone sequence, miR-128 backbone sequence, miR-144 backbone sequence, miR-451a backbone sequence, or miR-223 backbone sequence is modified (e.g., has nucleotide insertion, deletion, substitution, mismatch, wobble, or any combination thereof).

Sequence motifs that enable efficient processing of pri-miRNA backbones have previously been identified. These include an UG motif at the 5′ end of the pre-miRNA, a mismatched GHG motif in the stem, and a 3′ CNNC motif. In some embodiments, the miR backbone sequence has been modified to incorporate these motifs, including for example, miR-155E backbone sequence, miR-1-1_M backbone, miR-100_M backbone sequence, miR-124_M backbone sequence, and miR-122_M backbone sequence. Such modified miR backbones are labeled herein by the suffix “_M.”

In some embodiments, the miRNA (pri-miRNA, a pre-mRNA, an artificial miRNA, or a mature miRNA) comprises or consists of a guide strand sequence and corresponding passenger strand sequence of any one of the duplexe sequences set forth in Tables 1, 19, 23, and 24. In some embodiments, the passenger strand sequence of the miRNA comprises a sequence that is 100% complementary or perfectly complementary to the guide strand sequence. For example, a guide strand sequence may comprise or consist of a sequence of SEQ ID NO: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, or 436 (guide sequences in Table 1), and the passenger strand sequence may comprise or consist of a sequence of SEQ ID NO: 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, or 435 (passenger sequences in Table 1), respectively. In some embodiments, the passenger strand sequence of the miRNA is not 100% complementary or to the guide strand sequence. For example, a guide strand sequence may comprise or consist of a sequence of SEQ ID NO:1176 and the corresponding passenger strand sequence may comprise or consist of a sequence of SEQ ID NO:1289 (see, Table 19).

In some embodiments, the miRNA (pri-miRNA, a pre-mRNA, an artificial miRNA, or a mature miRNA) comprises a guide strand sequence comprising or consisting of any one of SEQ ID NOS: 12, 14, 40, 60, 100, 104, 108, 112, 124, 126, 128, 166, 198, 220, 242, 302, 306, 308, 330, 336, and 362, and a passenger strand sequence of comprising a sequence that is 100% complementary or perfectly complementary to the guide strand sequence. For example, a guide strand sequence may comprise or consist of a sequence of SEQ ID NO: 12, 14, 40, 60, 100, 104, 108, 112, 124, 126, 128, 166, 198, 220, 242, 302, 306, 308, 330, 336, or 362, and the passenger strand sequence may comprise or consist of a sequence of SEQ ID NO: 11, 13, 39, 59, 99, 103, 107, 111, 123, 125, 127, 165, 197, 219, 241, 301, 305, 307, 329, 335, or 361, respectively.

In some embodiments, the miRNA (pri-miRNA, a pre-mRNA, an artificial miRNA, or a mature miRNA) comprises a guide strand sequence comprising or consisting of any one of SEQ ID NOS: 14, 40, 100, 108, 112, 128, 166, 198, 242, 308, 336, and 362, and the passenger strand sequence of the miRNA comprises or consists of a sequence that is 100% complementary or perfectly complementary to the guide strand. For example, a guide strand sequence may comprise a sequence of SEQ ID NO: 14, 40, 100, 108, 112, 128, 166, 198, 242, 308, 336, or 362, and the passenger strand sequence may comprise a sequence of SEQ ID NO: 13, 39, 99, 107, 111, 127, 165, 197, 241, 307, 335, or 361, respectively.

In some embodiments, the miRNA (pri-miRNA, a pre-mRNA, an artificial miRNA, or a mature miRNA) comprises a guide strand sequence comprising or consisting of any one of the guide sequences of Tables 1, 19, 23, and 24 and the passenger strand sequence comprises or consists of a corresponding passenger sequence of Tables 1, 19, 23, and 24 that has 1, 2, 3, 4, 5, 6, 7, 8, 9, 10, or more insertions, deletions, substitutions, mismatches, wobbles, or any combination thereof relative to the passenger strand sequence of Tables 1, 19, 23 and 24. In some embodiments, the miRNA (pri-miRNA, a pre-mRNA, an artificial miRNA, or a mature miRNA) comprises a guide strand sequence comprising or consisting of any one of SEQ ID NOs: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, and a passenger strand sequence comprising or consisting a sequence of SEQ ID NOS: 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, respectively, wherein the passenger strand sequence has 1, 2, 3, 4, 5, 6, 7, 8, 9, 10, or more insertions, deletions, substitutions, mismatches, wobbles, or any combination thereof relative to the passenger strand sequence of SEQ ID NOS: 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, respectively. In some embodiments, a mismatch is a G→C, C→G, A→T, or T→A conversion in the passenger strand sequence. In some embodiments, a mismatch (to create a bulge with the guide strand) is a G→T, C→A, A→C, or T→G conversion in the passenger strand sequence. In some embodiments, a wobble is a G-U wobble, wherein a C is converted to a T in the passenger strand sequence. In some embodiments, the passenger strand sequence is modified according to the rules of Table 8.

In some embodiments, the miRNA (pri-miRNA, a pre-mRNA, an artificial miRNA, or a mature miRNA) comprises a guide strand sequence comprising or consisting of any one of SEQ ID NOS: 12, 14, 40, 60, 100, 104, 108, 112, 124, 126, 128, 166, 198, 220, 242, 302, 306, 308, 330, 336, and 362, and a passenger strand sequence comprising or consisting of a sequence that has 1, 2, 3, 4, 5, 6, 7, 8, 9, 10, or more insertions, deletions, substitutions, mismatches, wobbles, or any combination thereof relative to the passenger strand sequence comprising or consisting of a sequence of SEQ ID NOS: 11, 13, 39, 59, 99, 103, 107, 11, 123, 125, 127, 165, 197, 219, 241, 301, 305, 307, 329, 335, and 361, respectively. In some embodiments, a mismatch is a G→C, C→G, A→T, or T→A conversion in the passenger strand sequence. In some embodiments, a mismatch (to create a bulge with the guide strand) is a G→T, C→A, A→C, or T→G conversion in the passenger strand sequence. In some embodiments, a wobble is a G-U wobble, wherein a C is converted to a T in the passenger strand sequence. In some embodiments, the passenger strand sequence is modified according to the rules of Table 8.

In some embodiments, the miRNA (pri-miRNA, a pre-mRNA, an artificial miRNA, or a mature miRNA) comprises a guide strand sequence comprising or consisting of any one of SEQ ID NOS: 14, 40, 100, 108, 112, 128, 166, 198, 242, 308, 336, and 362, and a passenger strand sequence comprising or consisting of a sequence that has 1, 2, 3, 4, 5, 6, 7, 8, 9, 10, or more insertions, deletions, substitutions, mismatches, wobbles, or any combination thereof relative to the passenger strand sequence comprising or consisting of a sequence of SEQ ID NO: 13, 39, 99, 107, 111, 127, 165, 197, 241, 307, 335, or 361, respectively. In some embodiments, a mismatch is a G→C, C→G, A→T, or T→A conversion in the passenger strand sequence. In some embodiments, a mismatch (to create a bulge with the guide strand) is a G→T, C→A, A→C, or T→G conversion in the passenger strand sequence. In some embodiments, a wobble is a G-U wobble, wherein a C is converted to a T in the passenger strand sequence. In some embodiments, the passenger strand sequence is modified according to the rules of Table 8.

In some embodiments, the miRNA is an artificial miRNA comprising a guide strand sequence according to any of the embodiments described herein, contained within a miR-155 backbone sequence, miR1-1 backbone sequence, miR-100 backbone sequence, miR-124 backbone sequence, mIR-138-2 backbone sequence, miR-122 backbone sequence, miR-128 backbone sequence, miR-130a backbone sequence, or miR-16-2 backbone sequence, wherein the artificial miRNA comprises a passenger strand sequence that is modified according to Table 8. In some embodiments, the passenger strand sequence comprises a mismatch, wherein a mismatch is a G→C, C→G, A→T, or T→A conversion in the passenger strand sequence; a mismatch (to create a bulge with the guide strand) is a G→T, C→A, A→C, or T→G conversion in the passenger strand sequence; and a wobble is a G-U wobble, wherein a C is converted to a T in the passenger strand sequence.

In some embodiments, an artificial miRNA comprises or consists of a nucleic acid sequence set forth in any one of Tables 3, 9, 11, 19, 23, 24, and 25. In some embodiments, an artificial miRNA comprises or consists of a nucleic acid sequence of any one of SEQ ID NOS: 443-490, 1109-1111, 1114, 1121-1168, 1405-1520, 1908-2007, 2011, 2017, 2021, 2025, 2027, 2031, 2035, 2039, 2041, 2045, 2049, 2053, 2057, 2061, 2067, 2071, 2075, 2079, 2085, 2089, 2093, 2097, 2101, 2105, 2109, 2113, 2117, 2120, 2124, 2128, 2132, 2136, 2140, 2144, 2148, 2154, 2158, 2162, 2166, 2170, 2174, 2176, 2180, 2182, 2184, 2187, 2189, 2191, 2193, 2195, 2197, 2199, 2205, 2211, 2261, 2263, 2265, and 2267.

TABLE 3
ATXN2 Specific amiRNAs

| | Parent | miR | | |
|---|---|---|---|---|
| Parent Guide Sequence | Duplex ID | Backbone | Category | amiRNA Sequence |
| AGGAACGUGGGUUGAACUCCUU | XD-14857 | miR-1-1 | 911 Control | CAUGCAGACUGCCUGCUUGGGAUGGAGUUCAAGGGACGUCGCC |
| [SEQ ID NO: 242] | | | | UUAUGGACCUGCUAAGCUAAGGAACGUCCCUUGAACUCCUUCU |
| | | | | CAGGCCGGGACCUCUCUCGCCGCACUGAGGGGCACUCCACACC |
| | | | | ACGGGGGCC |
| | | | | [SEQ ID NO: 437] |
| AGGAACGUGGGUUGAACUCCUU | XD-14857 | miR-155E | 911 Control | CUGGAGGCUUGCUUUGGGCUGUAUGCUGAGGAACGUCCCUUGA |
| [SEQ ID NO: 242] | | | | ACUCCUUUUUUGGCCUCUGACUGAAAGGAGUUAAGGACGUUCC |
| | | | | UCAGGACAAGGCCCUUUAUCAGCACUCACAUGGAACAAAUGGC |
| | | | | CACCGUG |
| | | | | [SEQ ID NO: 438] |
| UUCGGGUUGAAAUCUGAAGUGU | XD-14790 | miR-155E | 911 Control | CUGGAGGCUUGCUUUGGGCUGUAUGCUGUUCGGGUUCUUAUCU |
| [SEQ ID NO: 108] | | | | GAAGUGUUUUUGGCCUCUGACUGAACACUUCAAUAGAACCCGA |
| | | | | ACAGGACAAGGCCCUUUAUCAGCACUCACAUGGAACAAAUGGC |
| | | | | CACCGUG |
| | | | | [SEQ ID NO: 439] |
| UUCGGGUUGAAAUCUGAAGUGU | XD-14790 | miR-1-1 | 911 Control | CAUGCAGACUGCCUGCUUGGGAGACUUCAGAUAAGAACCGAGA |
| [SEQ ID NO: 108] | | | | AUAUGGACCUGCUAAGCUAUUCGGGUUCUUAUCUGAAGUGUCU |
| | | | | CAGGCCGGGACCUCUCUCGCCGCACUGAGGGGCACUCCACACC |
| | | | | ACGGGGGCC |
| | | | | [SEQ ID NO: 440] |
| UUGAUUUCGAGGAUGUCGCUGG | XD-14800 | miR-155E | 911 Control | CUGGAGGCUUGCUUUGGGCUGUAUGCUGUUGAUUUCCUCGAUG |
| [SEQ ID NO: 128] | | | | UCGCUGGUUUUGGCCUCUGACUGACCAGCGACUCGGGAAAUCA |
| | | | | ACAGGACAAGGCCCUUUAUCAGCACUCACAUGGAACAAAUGGC |
| | | | | CACCGUG |
| | | | | [SEQ ID NO: 441] |
| UUGAUUUCGAGGAUGUCGCUGG | XD-14800 | miR-1-1 | 911 Control | CAUGCAGACUGCCUGCUUGGGCGAGCGACAUCGAGGAAACGCA |
| [SEQ ID NO: 128] | | | | AUAUGGACCUGCUAAGCUAUUGAUUUCCUCGAUGUCGCUGGCU |
| | | | | CAGGCCGGGACCUCUCUCGCCGCACUGAGGGGCACUCCACACC |
| | | | | ACGGGGGCC |
| | | | | [SEQ ID NO: 442] |
| AGAAAUCGUAGACUGAGGCAGU | XD-14743 | miR-1-1 | Atxn2 | CAUGCAGACUGCCUGCUUGGGAGUGCCUCAGUCUACGAUCGUC |
| [SEQ ID NO: 14] | | | targeting | UUAUGGACCUGCUAAGCUAAGAAAUCGUAGACUGAGGCAGUCU |
| | | | | CAGGCCGGGACCUCUCUCGCCGCACUGAGGGGCACUCCACACC |
| | | | | ACGGGGGCC |
| | | | | [SEQ ID NO: 443] |
| AGAAAUCGUAGACUGAGGCAGU | XD-14743 | miR-155E | Atxn2 | CUGGAGGCUUGCUUUGGGCUGUAUGCUGAGAAAUCGUAGACUG |
| [SEQ ID NO: 14] | | | targeting | AGGCAGUUUUUGGCCUCUGACUGAACUGCCUCGUCACGAUUUC |
| | | | | UCAGGACAAGGCCCUUUAUCAGCACUCACAUGGAACAAAUGGC |
| | | | | CACCGUG |
| | | | | [SEQ ID NO: 444] |
| AGAUACGUCAUUUUCCAAAGCC | XD-14766 | miR-1-1 | Atxn2 | CAUGCAGACUGCCUGCUUGGGGCCUUUGGAAAAUGACGUCCUC |
| [SEQ ID NO: 60] | | | targeting | UUAUGGACCUGCUAAGCUAAGAUACGUCAUUUUCCAAAGCCCU |
| | | | | CAGGCCGGGACCUCUCUCGCCGCACUGAGGGGCACUCCACACC |
| | | | | ACGGGGGCC |
| | | | | [SEQ ID NO: 445] |
| AGAUACGUCAUUUUCCAAAGCC | XD-14766 | miR-155E | Atxn2 | CUGGAGGCUUGCUUUGGGCUGUAUGCUGAGAUACGUCAUUUUC |
| [SEQ ID NO: 60] | | | targeting | CAAAGCCUUUUGGCCUCUGACUGAGGCUUUGGAAAGACGUAUC |
| | | | | UCAGGACAAGGCCCUUUAUCAGCACUCACAUGGAACAAAUGGC |
| | | | | CACCGUG |
| | | | | [SEQ ID NO: 446] |
| AGCGUUAGGGUGCGCAUACUGC | XD-14904 | miR-155E | Atxn2 | CUGGAGGCUUGCUUUGGGCUGUAUGCUGAGCGUUAGGGUGCGC |
| [SEQ ID NO : 336] | | | targeting | AUACUGCUUUUGGCCUCUGACUGAGCAGUAUGGCACCUAACGC |
| | | | | UCAGGACAAGGCCCUUUAUCAGCACUCACAUGGAACAAAUGGC |
| | | | | CACCGUG |
| | | | | [SEQ ID NO: 447] |
| AGCGUUAGGGUGCGCAUACUGC | XD-14904 | miR-1-1 | Atxn2 | CAUGCAGACUGCCUGCUUGGGGGAGUAUGCGCACCCUAAGAGC |
| [SEQ ID NO: 336] | | | targeting | UUAUGGACCUGCUAAGCUAAGCGUUAGGGUGCGCAUACUGCCU |
| | | | | CAGGCCGGGACCUCUCUCGCCGCACUGAGGGGCACUCCACACC |
| | | | | ACGGGGGCC |
| | | | | [SEQ ID NO: 448] |
| AGGAACGUGGGUUGAACUCCUU | XD-14857 | miR-155E | Atxn2 | CUGGAGGCUUGCUUUGGGCUGUAUGCUGAGGAACGUGGGUUGA |
| [SEQ ID NO: 242] | | | targeting | ACUCCUUUUUUGGCCUCUGACUGAAAGGAGUUAACCACGUUCC |
| | | | | UCAGGACAAGGCCCUUUAUCAGCACUCACAUGGAACAAAUGGC |
| | | | | CACCGUG |
| | | | | [SEQ ID NO: 449] |
| AGGAACGUGGGUUGAACUCCUU | XD-14857 | miR-1-1 | Atxn2 | CAUGCAGACUGCCUGCUUGGGAUGGAGUUCAACCCACGUCGCC |
| [SEQ ID NO: 242] | | | targeting | UUAUGGACCUGCUAAGCUAAGGAACGUGGGUUGAACUCCUUCU |
| | | | | CAGGCCGGGACCUCUCUCGCCGCACUGAGGGGCACUCCACACC |
| | | | | ACGGGGGCC |
| | | | | [SEQ ID NO: 450] |
| AUAAUAAUCCGUCAGUUUGACG | XD-14949 | miR-155E | Atxn2 | CUGGAGGCUUGCUUUGGGCUGUAUGCUGAUAAUAAUCCGUCAG |
| [SEQ ID NO: 426] | | | targeting | UUUGACGUUUUGGCCUCUGACUGACGUCAAACGACGAUUAUUA |
| | | | | UCAGGACAAGGCCCUUUAUCAGCACUCACAUGGAACAAAUGGC |
| | | | | CACCGUG |
| | | | | [SEQ ID NO: 451] |
| AUAAUAAUCCGUCAGUUUGACG | XD-14949 | miR-1-1 | Atxn2 | CAUGCAGACUGCCUGCUUGGGCCUCAAACUGACGGAUUACGUA |
| [SEQ ID NO: 426] | | | targeting | UUAUGGACCUGCUAAGCUAAUAAUAAUCCGUCAGUUUGACGCU |
| | | | | CAGGCCGGGACCUCUCUCGCCGCACUGAGGGGCACUCCACACC |
| | | | | ACGGGGGCC |
| | | | | [SEQ ID NO: 452] |
| AUACGCGGUGAAUUCUGUCUCC | XD-14787 | miR-155E | Atxn2 | CUGGAGGCUUGCUUUGGGCUGUAUGCUGAUACGCGGUGAAUUC |
| [SEQ ID NO: 102] | | | targeting | UGUCUCCUUUUGGCCUCUGACUGAGGAGACAGAUUACCGCGUA |
| | | | | UCAGGACAAGGCCCUUUAUCAGCACUCACAUGGAACAAAUGGC |
| | | | | CACCGUG |
| | | | | [SEQ ID NO: 453] |
| AUACGCGGUGAAUUCUGUCUCC | XD-14787 | miR-1-1 | Atxn2 | CAUGCAGACUGCCUGCUUGGGGCAGACAGAAUUCACCGCCUUA |
| [SEQ ID NO: 102] | | | targeting | UUAUGGACCUGCUAAGCUAAUACGCGGUGAAUUCUGUCUCCCU |
| | | | | CAGGCCGGGACCUCUCUCGCCGCACUGAGGGGCACUCCACACC |
| | | | | ACGGGGGCC |
| | | | | [SEQ ID NO: 454] |
| AUUAACUACUCUUUGGUCUGAA | XD-14792 | miR-1-1 | Atxn2 | CAUGCAGACUGCCUGCUUGGGUACAGACCAAAGAGUAGUCGAA |
| [SEQ ID NO: 112] | | | targeting | UUAUGGACCUGCUAAGCUAAUUAACUACUCUUUGGUCUGAACU |
| | | | | CAGGCCGGGACCUCUCUCGCCGCACUGAGGGGCACUCCACACC |
| | | | | ACGGGGGCC |
| | | | | [SEQ ID NO: 455] |
| AUUAACUACUCUUUGGUCUGAA | XD-14792 | miR-155E | Atxn2 | CUGGAGGCUUGCUUUGGGCUGUAUGCUGAUUAACUACUCUUUG |
| [SEQ ID NO: 112] | | | targeting | GUCUGAAUUUUGGCCUCUGACUGAUUCAGACCAAGGUAGUUAA |
| | | | | UCAGGACAAGGCCCUUUAUCAGCACUCACAUGGAACAAAUGGC |
| | | | | CACCGUG |
| | | | | [SEQ ID NO: 456] |
| AUUGCGUGGAGUAAGCUGGUGG | XD-14889 | miR-155E | Atxn2 | CUGGAGGCUUGCUUUGGGCUGUAUGCUGAUUGCGUGGAGUAAG |
| [SEQ ID NO: 306] | | | targeting | CUGGUGGUUUUGGCCUCUGACUGACCACCAGCUACCCACGCAA |
| | | | | UCAGGACAAGGCCCUUUAUCAGCACUCACAUGGAACAAAUGGC |
| | | | | CACCGUG |
| | | | | [SEQ ID NO: 457] |
| AUUGCGUGGAGUAAGCUGGUGG | XD-14889 | miR-1-1 | Atxn2 | CAUGCAGACUGCCUGCUUGGGCGACCAGCUUACUCCACGGAAA |
| [SEQ ID NO: 306] | | | targeting | UUAUGGACCUGCUAAGCUAAUUGCGUGGAGUAAGCUGGUGGCU |
| | | | | CAGGCCGGGACCUCUCUCGCCGCACUGAGGGGCACUCCACACC |
| | | | | ACGGGGGCC |
| | | | | [SEQ ID NO: 458] |
| AUUUCGAGGAUGUCGCUGGGCC | XD-14798 | miR-155E | Atxn2 | CUGGAGGCUUGCUUUGGGCUGUAUGCUGAUUUCGAGGAUGUCG |
| [SEQ ID NO: 124] | | | targeting | CUGGGCCUUUUGGCCUCUGACUGAGGCCCAGCACACCUCGAAA |
| | | | | UCAGGACAAGGCCCUUUAUCAGCACUCACAUGGAACAAAUGGC |
| | | | | CACCGUG |
| | | | | [SEQ ID NO: 459] |
| AUUUCGAGGAUGUCGCUGGGCC | XD-14798 | miR-1-1 | Atxn2 | CAUGCAGACUGCCUGCUUGGGGCCCCAGCGACAUCCUCGCCAA |
| [SEQ ID NO: 124] | | | targeting | UUAUGGACCUGCUAAGCUAAUUUCGAGGAUGUCGCUGGGCCCU |
| | | | | CAGGCCGGGACCUCUCUCGCCGCACUGAGGGGCACUCCACACC |
| | | | | ACGGGGGCC |
| | | | | [SEQ ID NO: 460] |
| UAAAUCGUAGACUGAGGCAGUC | XD-14742 | miR-1-1 | Atxn2 | CAUGCAGACUGCCUGCUUGGGGUCUGCCUCAGUCUACGACGUU |
| [SEQ ID NO: 12] | | | targeting | AUAUGGACCUGCUAAGCUAUAAAUCGUAGACUGAGGCAGUCCU |
| | | | | CAGGCCGGGACCUCUCUCGCCGCACUGAGGGGCACUCCACACC |
| | | | | ACGGGGGCC |
| | | | | [SEQ ID NO: 461] |
| UAAAUCGUAGACUGAGGCAGUC | XD-14742 | miR-155E | Atxn2 | CUGGAGGCUUGCUUUGGGCUGUAUGCUGUAAAUCGUAGACUGA |
| [SEQ ID NO: 12] | | | targeting | GGCAGUCUUUUGGCCUCUGACUGAGACUGCCUAGUUACGAUUU |
| | | | | ACAGGACAAGGCCCUUUAUCAGCACUCACAUGGAACAAAUGGC |
| | | | | CACCGUG |
| | | | | [SEQ ID NO: 462] |
| UACGCGGUGAAUUCUGUCUCCC | XD-14786 | miR-1-1 | Atxn2 | CAUGCAGACUGCCUGCUUGGGGCGAGACAGAAUUCACCGGAGU |
| [SEQ ID NO: 100] | | | targeting | AUAUGGACCUGCUAAGCUAUACGCGGUGAAUUCUGUCUCCCCU |
| | | | | CAGGCCGGGACCUCUCUCGCCGCACUGAGGGGCACUCCACACC |
| | | | | ACGGGGGCC |
| | | | | [SEQ ID NO: 463] |
| UACGCGGUGAAUUCUGUCUCCC | XD-14786 | miR-155E | Atxn2 | CUGGAGGCUUGCUUUGGGCUGUAUGCUGUACGCGGUGAAUUCU |
| [SEQ ID NO: 100] | | | targeting | GUCUCCCUUUUGGCCUCUGACUGAGGGAGACAAAUCACCGCGU |
| | | | | ACAGGACAAGGCCCUUUAUCAGCACUCACAUGGAACAAAUGGC |
| | | | | CACCGUG |
| | | | | [SEQ ID NO: 464] |
| UAUACGCGGUGAAUUCUGUCUC | XD-14788 | miR-1-1 | Atxn2 | CAUGCAGACUGCCUGCUUGGGGUGACAGAAUUCACCGCGCGAU |
| [SEQ ID NO: 104] | | | targeting | AUAUGGACCUGCUAAGCUAUAUACGCGGUGAAUUCUGUCUCCU |
| | | | | CAGGCCGGGACCUCUCUCGCCGCACUGAGGGGCACUCCACACC |
| | | | | ACGGGGGCC |
| | | | | [SEQ ID NO: 465] |
| UAUACGCGGUGAAUUCUGUCUC | XD-14788 | miR-155E | Atxn2 | CUGGAGGCUUGCUUUGGGCUGUAUGCUGUAUACGCGGUGAAUU |
| [SEQ ID NO: 104] | | | targeting | CUGUCUCUUUUGGCCUCUGACUGAGAGACAGAUUCCCGCGUAU |
| | | | | ACAGGACAAGGCCCUUUAUCAGCACUCACAUGGAACAAAUGGC |
| | | | | CACCGUG |
| | | | | [SEQ ID NO: 466] |
| UAUUGCGUGGAGUAAGCUGGUG | XD-14890 | miR-1-1 | Atxn2 | CAUGCAGACUGCCUGCUUGGGCUCCAGCUUACUCCACGCCCAU |
| [SEQ ID NO: 308] | | | targeting | AUAUGGACCUGCUAAGCUAUAUUGCGUGGAGUAAGCUGGUGCU |
| | | | | CAGGCCGGGACCUCUCUCGCCGCACUGAGGGGCACUCCACACC |
| | | | | ACGGGGGCC |
| | | | | [SEQ ID NO: 467] |
| UAUUGCGUGGAGUAAGCUGGUG | XD-14890 | miR-155E | Atxn2 | CUGGAGGCUUGCUUUGGGCUGUAUGCUGUAUUGCGUGGAGUAA |
| [SEQ ID NO: 308] | | | targeting | GCUGGUGUUUUGGCCUCUGACUGACACCAGCUACUCACGCAAU |
| | | | | ACAGGACAAGGCCCUUUAUCAGCACUCACAUGGAACAAAUGGC |
| | | | | CACCGUG |
| | | | | [SEQ ID NO: 468] |
| UAUUUCGAGGAUGUCGCUGGGC | XD-14799 | miR-155E | Atxn2 | CUGGAGGCUUGCUUUGGGCUGUAUGCUGUAUUUCGAGGAUGUC |
| [SEQ ID NO: 126] | | | targeting | GCUGGGCUUUUGGCCUCUGACUGAGCCCAGCGCAUCUCGAAAU |
| | | | | ACAGGACAAGGCCCUUUAUCAGCACUCACAUGGAACAAAUGGC |
| | | | | CACCGUG |
| | | | | [SEQ ID NO: 469] |
| UAUUUCGAGGAUGUCGCUGGGC | XD-14799 | miR-1-1 | Atxn2 | CAUGCAGACUGCCUGCUUGGGGGCCAGCGACAUCCUCGACCAU |
| [SEQ ID NO: 126] | | | targeting | AUAUGGACCUGCUAAGCUAUAUUUCGAGGAUGUCGCUGGGCCU |
| | | | | CAGGCCGGGACCUCUCUCGCCGCACUGAGGGGCACUCCACACC |
| | | | | ACGGGGGCC |
| | | | | [SEQ ID NO: 470] |
| UCGCUGUUGGGGCAUAUUUGGU | XD-14887 | miR-1-1 | Atxn2 | CAUGCAGACUGCCUGCUUGGGAGCAAAUAUGCCCCAACACUCG |
| [SEQ ID NO: 302] | | | targeting | AUAUGGACCUGCUAAGCUAUCGCUGUUGGGGCAUAUUUGGUCU |
| | | | | CAGGCCGGGACCUCUCUCGCCGCACUGAGGGGCACUCCACACC |
| | | | | ACGGGGGCC |
| | | | | [SEQ ID NO: 471] |
| UCGCUGUUGGGGCAUAUUUGGU | XD-14887 | miR-155E | Atxn2 | CUGGAGGCUUGCUUUGGGCUGUAUGCUGUCGCUGUUGGGGCAU |
| [SEQ ID NO: 302] | | | targeting | AUUUGGUUUUUGGCCUCUGACUGAACCAAAUAGCCCAACAGCG |
| | | | | ACAGGACAAGGCCCUUUAUCAGCACUCACAUGGAACAAAUGGC |
| | | | | CACCGUG |
| | | | | [SEQ ID NO: 472] |
| UGCGCAUACUGCUGAGCAAGGG | XD-14901 | miR-1-1 | Atxn2 | CAUGCAGACUGCCUGCUUGGGCGCUUGCUCAGCAGUAUGGAGC |
| [SEQ ID NO: 330] | | | targeting | AUAUGGACCUGCUAAGCUAUGCGCAUACUGCUGAGCAAGGGCU |
| | | | | CAGGCCGGGACCUCUCUCGCCGCACUGAGGGGCACUCCACACC |
| | | | | ACGGGGGCC |
| | | | | [SEQ ID NO: 473] |
| UGCGCAUACUGCUGAGCAAGGG | XD-14901 | miR-155E | Atxn2 | CUGGAGGCUUGCUUUGGGCUGUAUGCUGUGCGCAUACUGCUGA |
| [SEQ ID NO: 330] | | | targeting | GCAAGGGUUUUGGCCUCUGACUGACCCUUGCUAGCGUAUGCGC |
| | | | | ACAGGACAAGGCCCUUUAUCAGCACUCACAUGGAACAAAUGGC |
| | | | | CACCGUG |
| | | | | [SEQ ID NO: 474] |
| UGUACCACAACAAAGUCUGAAC | XD-14756 | miR-1-1 | Atxn2 | CAUGCAGACUGCCUGCUUGGGGAUCAGACUUUGUUGUGGCGAC |
| [SEQ ID NO: 40] | | | targeting | AUAUGGACCUGCUAAGCUAUGUACCACAACAAAGUCUGAACCU |
| | | | | CAGGCCGGGACCUCUCUCGCCGCACUGAGGGGCACUCCACACC |
| | | | | ACGGGGGCC |
| | | | | [SEQ ID NO: 475] |
| UGUACCACAACAAAGUCUGAAC | XD-14756 | miR-155E | Atxn2 | CUGGAGGCUUGCUUUGGGCUGUAUGCUGUGUACCACAACAAAG |
| [SEQ ID NO: 40] | | | targeting | UCUGAACUUUUGGCCUCUGACUGAGUUCAGACUUGUGUGGUAC |
| | | | | ACAGGACAAGGCCCUUUAUCAGCACUCACAUGGAACAAAUGGC |
| | | | | CACCGUG |
| | | | | [SEQ ID NO: 476] |
| UGUAUACGCCGGCUGAACGUGA | XD-14917 | miR-1-1 | Atxn2 | CAUGCAGACUGCCUGCUUGGGUGACGUUCAGCCGGCGUACGAC |
| [SEQ ID NO: 362] | | | targeting | AUAUGGACCUGCUAAGCUAUGUAUACGCCGGCUGAACGUGACU |
| | | | | CAGGCCGGGACCUCUCUCGCCGCACUGAGGGGCACUCCACACC |
| | | | | ACGGGGGCC |
| | | | | [SEQ ID NO: 477] |
| UGUAUACGCCGGCUGAACGUGA | XD-14917 | miR-155E | Atxn2 | CUGGAGGCUUGCUUUGGGCUGUAUGCUGUGUAUACGCCGGCUG |
| [SEQ ID NO: 362] | | | targeting | AACGUGAUUUUGGCCUCUGACUGAUCACGUUCGCCGCGUAUAC |
| | | | | ACAGGACAAGGCCCUUUAUCAGCACUCACAUGGAACAAAUGGC |
| | | | | CACCGUG |
| | | | | [SEQ ID NO: 478] |
| UUACUAAGUAUUGAAGGGGAAA | XD-14846 | miR-155E | Atxn2 | CUGGAGGCUUGCUUUGGGCUGUAUGCUGUUACUAAGUAUUGAA |
| [SEQ ID NO: 220] | | | targeting | GGGGAAAUUUUGGCCUCUGACUGAUUUCCCCUCAAACUUAGUA |
| | | | | ACAGGACAAGGCCCUUUAUCAGCACUCACAUGGAACAAAUGGC |
| | | | | CACCGUG |
| | | | | [SEQ ID NO: 479] |
| UUACUAAGUAUUGAAGGGGAAA | XD-14846 | miR-1-1 | Atxn2 | CAUGCAGACUGCCUGCUUGGGUAUCCCCUUCAAUACUUACUUA |
| [SEQ ID NO: 220] | | | targeting | AUAUGGACCUGCUAAGCUAUUACUAAGUAUUGAAGGGGAAACU |
| | | | | CAGGCCGGGACCUCUCUCGCCGCACUGAGGGGCACUCCACACC |
| | | | | ACGGGGGCC |
| | | | | [SEQ ID NO: 480] |
| UUAGUUGAUCCAUAGAUUCAGA | XD-14835 | miR-1-1 | Atxn2 | CAUGCAGACUGCCUGCUUGGGUGUGAAUCUAUGGAUCAAGAUA |
| [SEQ ID NO: 198] | | | targeting | AUAUGGACCUGCUAAGCUAUUAGUUGAUCCAUAGAUUCAGACU |
| | | | | CAGGCCGGGACCUCUCUCGCCGCACUGAGGGGCACUCCACACC |
| | | | | ACGGGGGCC |
| | | | | [SEQ ID NO: 481] |
| UUAGUUGAUCCAUAGAUUCAGA | XD-14835 | miR-155E | Atxn2 | CUGGAGGCUUGCUUUGGGCUGUAUGCUGUUAGUUGAUCCAUAG |
| [SEQ ID NO: 198] | | | targeting | AUUCAGAUUUUGGCCUCUGACUGAUCUGAAUCAUGAUCAACUA |
| | | | | ACAGGACAAGGCCCUUUAUCAGCACUCACAUGGAACAAAUGGC |
| | | | | CACCGUG |
| | | | | [SEQ ID NO: 482] |
| UUCGAUGCAGGACUAGCAGGCG | XD-14819 | miR-155E | Atxn2 | CUGGAGGCUUGCUUUGGGCUGUAUGCUGUUCGAUGCAGGACUA |
| [SEQ ID NO: 166] | | | targeting | GCAGGCGUUUUGGCCUCUGACUGACGCCUGCUGUCUGCAUCGA |
| | | | | ACAGGACAAGGCCCUUUAUCAGCACUCACAUGGAACAAAUGGC |
| | | | | CACCGUG |
| | | | | [SEQ ID NO: 483] |
| UUCGAUGCAGGACUAGCAGGCG | XD-14819 | miR-1-1 | Atxn2 | CAUGCAGACUGCCUGCUUGGGCCCCUGCUAGUCCUGCAUGAGA |
| [SEQ ID NO: 166] | | | targeting | AUAUGGACCUGCUAAGCUAUUCGAUGCAGGACUAGCAGGCGCU |
| | | | | CAGGCCGGGACCUCUCUCGCCGCACUGAGGGGCACUCCACACC |
| | | | | ACGGGGGCC |
| | | | | [SEQ ID NO: 484] |
| UUCGGGUUGAAAUCUGAAGUGU | XD-14790 | miR-155E | Atxn2 | CUGGAGGCUUGCUUUGGGCUGUAUGCUGUUCGGGUUGAAAUCU |
| [SEQ ID NO: 108] | | | targeting | GAAGUGUUUUUGGCCUCUGACUGAACACUUCAAUUCAACCCGA |
| | | | | ACAGGACAAGGCCCUUUAUCAGCACUCACAUGGAACAAAUGGC |
| | | | | CACCGUG |
| | | | | [SEQ ID NO: 485] |
| UUCGGGUUGAAAUCUGAAGUGU | XD-14790 | miR-1-1 | Atxn2 | CAUGCAGACUGCCUGCUUGGGAGACUUCAGAUUUCAACCGAGA |
| [SEQ ID NO: 108] | | | targeting | AUAUGGACCUGCUAAGCUAUUCGGGUUGAAAUCUGAAGUGUCU |
| | | | | CAGGCCGGGACCUCUCUCGCCGCACUGAGGGGCACUCCACACC |
| | | | | ACGGGGGCC |
| | | | | [SEQ ID NO: 486] |
| UUGAUUUCGAGGAUGUCGCUGG | XD-14800 | miR-1-1 | Atxn2 | CAUGCAGACUGCCUGCUUGGGCGAGCGACAUCCUCGAAACGCA |
| [SEQ ID NO: 128] | | | targeting | AUAUGGACCUGCUAAGCUAUUGAUUUCGAGGAUGUCGCUGGCU |
| | | | | CAGGCCGGGACCUCUCUCGCCGCACUGAGGGGCACUCCACACC |
| | | | | ACGGGGGCC |
| | | | | [SEQ ID NO: 487] |
| UUGAUUUCGAGGAUGUCGCUGG | XD-14800 | miR-155E | Atxn2 | CUGGAGGCUUGCUUUGGGCUGUAUGCUGUUGAUUUCGAGGAUG |
| [SEQ ID NO: 128] | | | targeting | UCGCUGGUUUUGGCCUCUGACUGACCAGCGACUCCCGAAAUCA |
| | | | | ACAGGACAAGGCCCUUUAUCAGCACUCACAUGGAACAAAUGGC |
| | | | | CACCGUG |
| | | | | [SEQ ID NO: 488] |
| UUGUACUGGGCACUUGACUCAA | XD-14781 | miR-155E | Atxn2 | CUGGAGGCUUGCUUUGGGCUGUAUGCUGUUGUACUGGGCACUU |
| [SEQ ID NO: 90] | | | targeting | GACUCAAUUUUGGCCUCUGACUGAUUGAGUCAGUGCCAGUACA |
| | | | | ACAGGACAAGGCCCUUUAUCAGCACUCACAUGGAACAAAUGGC |
| | | | | CACCGUG |
| | | | | [SEQ ID NO: 489] |
| UUGUACUGGGCACUUGACUCAA | XD-14781 | miR-1-1 | Atxn2 | CAUGCAGACUGCCUGCUUGGGUAGAGUCAAGUGCCCAGUCCCA |
| [SEQ ID NO: 90] | | | targeting | AUAUGGACCUGCUAAGCUAUUGUACUGGGCACUUGACUCAACU |
| | | | | CAGGCCGGGACCUCUCUCGCCGCACUGAGGGGCACUCCACACC |
| | | | | ACGGGGGCC |
| | | | | [SEQ ID NO: 490] |

In some embodiments, an artificial miRNA comprises or consists of a nucleic acid sequence set forth in Table 3. In some embodiments, an artificial miRNA comprises or consists of a nucleic acid sequence of any one of SEQ ID NOS:443-490.

In some embodiments, an artificial miRNA comprises or consists of a nucleic acid sequence set forth in Table 9. In some embodiments, an artificial miRNA comprises or consists of a nucleic acid sequence of any one of SEQ ID NOS:1109-1111, and 1114.

In some embodiments, an artificial miRNA comprises or consists of a nucleic acid sequence set forth in Table 11. In some embodiments, an artificial miRNA comprises or consists of a nucleic acid sequence of any one of SEQ ID NOS:1121-1168.

In some embodiments, an artificial miRNA comprises or consists of a nucleic acid sequence set forth in Table 19. In some embodiments, an artificial miRNA comprises or consists of a nucleic acid sequence of any one of SEQ ID NOS:1405-1520.

In some embodiments, an artificial miRNA comprises or consists of a nucleic acid sequence set forth in Table 23. In some embodiments, an artificial miRNA comprises or consists of a nucleic acid sequence of any one of SEQ ID NOS:1908-2007.

In some embodiments, an artificial miRNA comprises or consists of a nucleic acid sequence set forth in Table 24. In some embodiments, an artificial miRNA comprises or consists of a nucleic acid sequence of any one of SEQ ID NOS:1908-1934, 1936-1977, 1979-1982, 1984-1994, 1997, 1998, 2000, 2001, 2005-2007, 2011, 2017, 2021, 2025, 2027, 2031, 2035, 2039, 2041, 2045, 2049, 2053, 2057, 2061, 2067, 2071, 2075, 2079, 2085, 2089, 2093, 2097, 2101, 2105, 2109, 2113, 2117, 2120, 2124, 2128, 2132, 2136, 2140, 2144, 2148, 2154, 2158, 2162, 2166, 2170, 2174, 2176, 2180, 2182, 2184, 2187, 2189, 2191, 2193, 2195, 2197, 2199, 2205, 2211, 2261, 2263, 2265, and 2267.

In some embodiments, an artificial miRNA comprises or consists of a nucleic acid sequence set forth in Table 25. In some embodiments, an artificial miRNA comprises or consists of a nucleic acid sequence of any one of SEQ ID NOS:1915, 1982, 1965, 1937, 1985, 1921, and 2021.

### Expression Constructs

In another aspect, the present disclosure provides an isolated nucleic acid comprising an expression construct or expression cassette encoding any one of the inhibitory nucleic acids (e.g., siRNA, shRNA, dsRNA, miRNA, amiRNA, etc.) that inhibit the expression or activity of ATXN2 as described herein.

In some embodiments, the isolated nucleic acid comprises an expression construct encoding an inhibitory nucleic acid that inhibits the expression or activity of ATXN2, wherein the inhibitory nucleic acid comprises a guide strand sequence comprising or consisting of the nucleic acid sequence set forth in any one of the guide sequences of Tables 1, 3, 9, 11, 12, 13, 19, 23, 24, and 25, e.g., SEQ ID NOS: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 1176-1288, 1811-1827, 2015, 2065, 2083, 2152, 2203, and 2209.

In some embodiments, the isolated nucleic acid comprises an expression construct encoding an inhibitory nucleic acid that inhibits the expression or activity of ATXN2, wherein the inhibitory nucleic acid comprises a guide strand sequence comprising or consisting of the nucleic acid sequence set forth in any one of the guide sequences of Tables 1, 3, 9, 11, 12, 13, 19, 23, 24, and 25, e.g., SEQ ID NOS: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 1176-1288, 1811-1827, 2015, 2065, 2083, 2152, 2203, and 2209, with at least 1, 2, 3, 4, or 5 mismatches to the target ATXN2 mRNA sequence.

In some embodiments, the isolated nucleic acid comprises an expression construct encoding an inhibitory nucleic acid that inhibits the expression or activity of ATXN2, wherein the inhibitory nucleic acid comprises a guide strand sequence comprising or consisting of a sequence at least 60%, 70%, 75%, 80%, 85%, 90%, 95%, 97%, 99%, or 100% identical to any one of the guide sequences of Tables 1, 3, 9, 11, 12, 13, 19, 23, 24, and 25, e.g., SEQ ID NOS: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 1176-1288, 1811-1827, 2015, 2065, 2083, 2152, 2203, and 2209.

In some embodiments, the isolated nucleic acid comprises an expression construct encoding an inhibitory nucleic acid that inhibits the expression or activity of ATXN2, wherein the inhibitory nucleic acid comprises a guide strand sequence comprising or consisting of at least 15, 16, 17, 18, 19, 20, 21, or 22 contiguous nucleotides of a sequence of any one of the guide sequences of Tables 1, 3, 9, 11, 12, 13, 19, 23, 24, and 25 e.g., SEQ ID NOS: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 1176-1288, 1811-1827, 2015, 2065, 2083, 2152, 2203, and 2209, preferably wherein the guide strand sequence retains positions 2-7 (“seed sequence”) of the selected SEQ ID NO.

In some embodiments, the isolated nucleic acid comprises an expression construct encoding an inhibitory nucleic acid that inhibits the expression or activity of ATXN2, wherein the inhibitory nucleic acid comprises a guide strand sequence comprising or consisting of a sequence of any one of the guide sequences of Tables 1, 3, 9, 11, 12, 13, 19, 23, 24, and 25, e.g., SEQ ID NOS: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 1176-1288, 1811-1827, 2015, 2065, 2083, 2152, 2203, and 2209, wherein 1, 2, 3, or 4 nucleotides at positions 19-22 differ from the selected SEQ ID NO (variant nucleotide(s)) such that the guide strand sequence is no longer complementary to the ATXN2 target sequence at the variant nucleotide(s).

In some embodiments, the isolated nucleic acid molecule comprises an expression construct encoding an inhibitory nucleic acid that inhibits the expression or activity of ATXN2, wherein the inhibitory nucleic acid comprises a guide strand sequence of Table 12. In some embodiments, the inhibitory nucleic acid comprises a guide strand sequence comprising or consisting of the nucleic acid sequence set forth in any one of SEQ ID NOS: 12, 14, 40, 60, 100, 104, 108, 112, 124, 126, 128, 166, 198, 220, 242, 302, 306, 308, 330, 336, and 362. In some embodiments, the isolated nucleic acid comprises an expression construct encoding an inhibitory nucleic acid that inhibits the expression or activity of ATXN2, wherein the inhibitory nucleic acid comprises a guide strand sequence comprising or consisting of the nucleic acid sequence set forth in any one of SEQ ID NOS: 12, 14, 40, 60, 100, 104, 108, 112, 124, 126, 128, 166, 198, 220, 242, 302, 306, 308, 330, 336, and 362 with at least 1, 2, 3, 4, or 5 mismatches to the target ATXN2 mRNA sequence. In some embodiments, the isolated nucleic acid comprises an expression construct encoding an inhibitory nucleic acid that inhibits the expression or activity of ATXN2, wherein the inhibitory nucleic acid comprises a guide strand sequence comprising or consisting of a nucleic acid sequence that is at least 60%, 70%, 75%, 80%, 85%, 90%, 95%, 97%, 99%, or 100% identical to any one of SEQ ID NOS: 12, 14, 40, 60, 100, 104, 108, 112, 124, 126, 128, 166, 198, 220, 242, 302, 306, 308, 330, 336, and 362. In some embodiments, the isolated nucleic acid comprises an expression construct encoding an inhibitory nucleic acid that inhibits the expression or activity of ATXN2, wherein the inhibitory nucleic acid comprises a guide strand sequence comprising or consisting of at least 15, 16, 17, 18, 19, 20, 21, or 22 contiguous nucleotides of a sequence of any one of SEQ ID NOS: 12, 14, 40, 60, 100, 104, 108, 112, 124, 126, 128, 166, 198, 220, 242, 302, 306, 308, 330, 336, and 362, preferably wherein the guide strand sequence retains positions 2-7 (“seed sequence”) of the selected SEQ ID NO. In some embodiments, the isolated nucleic acid comprises an expression construct encoding an inhibitory nucleic acid that inhibits the expression or activity of ATXN2, wherein the inhibitory nucleic acid comprises a guide strand sequence comprising or consisting of a sequence of any one of SEQ ID NOS: 12, 14, 40, 60, 100, 104, 108, 112, 124, 126, 128, 166, 198, 220, 242, 302, 306, 308, 330, 336, and 362, wherein 1, 2, 3, or 4 nucleotides at positions 19-22 differ from the selected SEQ ID NO (variant nucleotide(s)), such that the guide strand sequence is no longer complementary to the ATXN2 target sequence at the variant nucleotide(s).

In some embodiments, the isolated nucleic acid comprises an expression construct encoding an inhibitory nucleic acid that inhibits the expression or activity of ATXN2, wherein the inhibitory nucleic acid comprises a guide strand sequence of Table 13. In some embodiments, the inhibitory nucleic acid comprises a guide strand sequence comprising or consisting of the nucleic acid sequence set forth in any one of SEQ ID NOS: 14, 40, 100, 108, 112, 128, 166, 198, 242, 308, 336, and 362. In some embodiments, the isolated nucleic acid comprises an expression construct encoding an inhibitory nucleic acid that inhibits the expression or activity of ATXN2, wherein the inhibitory nucleic acid comprises a guide strand sequence comprising or consisting of the nucleic acid sequence set forth in any one of SEQ ID NOS: 14, 40, 100, 108, 112, 128, 166, 198, 242, 308, 336, and 362 with at least 1, 2, 3, 4, or 5 mismatches to the target ATXN2 mRNA sequence. In some embodiments, the isolated nucleic acid comprises an expression construct encoding an inhibitory nucleic acid that inhibits the expression or activity of ATXN2, wherein the inhibitory nucleic acid comprises a guide strand sequence comprising or consisting of a nucleic acid sequence that is at least 60%, 70%, 75%, 80%, 85%, 90%, 95%, 97%, 99%, or 100% identical to any one of SEQ ID NOS: 14, 40, 100, 108, 112, 128, 166, 198, 242, 308, 336, and 362. In some embodiments, the isolated nucleic acid comprises an expression construct encoding an inhibitory nucleic acid that inhibits the expression or activity of ATXN2, wherein the inhibitory nucleic acid comprises a guide strand sequence comprising or consisting of at least 15, 16, 17, 18, 19, 20, 21, or 22 contiguous nucleotides of a sequence of any one of SEQ ID NOS: 14, 40, 100, 108, 112, 128, 166, 198, 242, 308, 336, and 362, preferably wherein the guide strand sequence retains positions 2-7 (“seed sequence”) of the selected SEQ ID NO. In some embodiments, the isolated nucleic acid comprises an expression construct encoding an inhibitory nucleic acid that inhibits the expression or activity of ATXN2, wherein the inhibitory nucleic acid comprises a guide strand sequence comprising or consisting of a sequence of any one of SEQ ID NOS: 14, 40, 100, 108, 112, 128, 166, 198, 242, 308, 336, and 362, wherein 1, 2, 3, or 4 nucleotides at positions 19-22 differ from the selected SEQ ID NO (variant nucleotide(s)), such that the guide strand sequence is no longer complementary to the ATXN2 target sequence at the variant nucleotide(s).

In some embodiments, the isolated nucleic acid comprises an expression construct encoding an inhibitory nucleic acid that inhibits the expression or activity of ATXN2, wherein the inhibitory nucleic acid comprises a guide strand sequence of Table 19. In some embodiments, the inhibitory nucleic acid comprises a guide strand sequence comprising or consisting of the nucleic acid sequence set forth in any one of SEQ ID NOS: 1176-1288, 40, 108, and 166. In some embodiments, the isolated nucleic acid comprises an expression construct encoding an inhibitory nucleic acid that inhibits the expression or activity of ATXN2, wherein the inhibitory nucleic acid comprises a guide strand sequence comprising or consisting of the nucleic acid sequence set forth in any one of SEQ ID NOS: 1176-1288, 40, 108, and 166 with at least 1, 2, 3, 4, or 5 mismatches to the target ATXN2 mRNA sequence. In some embodiments, the isolated nucleic acid comprises an expression construct encoding an inhibitory nucleic acid that inhibits the expression or activity of ATXN2, wherein the inhibitory nucleic acid comprises a guide strand sequence comprising or consisting of a nucleic acid sequence that is at least 60%, 70%, 75%, 80%, 85%, 90%, 95%, 97%, 99%, or 100% identical to any one of SEQ ID NOS:1176-1288, 40, 108, and 166. In some embodiments, the isolated nucleic acid comprises an expression construct encoding an inhibitory nucleic acid that inhibits the expression or activity of ATXN2, wherein the inhibitory nucleic acid comprises a guide strand sequence comprising or consisting of at least 15, 16, 17, 18, 19, 20, 21, or 22 contiguous nucleotides of a sequence of any one of SEQ ID NOS:1176-1288, 40, 108, and 166, preferably wherein the guide strand sequence retains positions 2-7 (“seed sequence”) of the selected SEQ ID NO. In some embodiments, the isolated nucleic acid comprises an expression construct encoding an inhibitory nucleic acid that inhibits the expression or activity of ATXN2, wherein the inhibitory nucleic acid comprises a guide strand sequence comprising or consisting of a sequence of any one of SEQ ID NOS:1176-1288, 40, 108, and 166, wherein 1, 2, 3, or 4 nucleotides at positions 19-22 differ from the selected SEQ ID NO (variant nucleotide(s)), such that the guide strand sequence is no longer complementary to the ATXN2 target sequence at the variant nucleotide(s).

In some embodiments, the isolated nucleic acid comprises an expression construct encoding an inhibitory nucleic acid that inhibits the expression or activity of ATXN2, wherein the inhibitory nucleic acid comprises a guide strand sequence of Table 23. In some embodiments, the inhibitory nucleic acid comprises a guide strand sequence comprising or consisting of the nucleic acid sequence set forth in any one of SEQ ID NOS:1908-2007. In some embodiments, the isolated nucleic acid comprises an expression construct encoding an inhibitory nucleic acid that inhibits the expression or activity of ATXN2, wherein the inhibitory nucleic acid comprises a guide strand sequence comprising or consisting of the nucleic acid sequence set forth in any one of SEQ ID NOS:1908-2007 with at least 1, 2, 3, 4, or 5 mismatches to the target ATXN2 mRNA sequence. In some embodiments, the isolated nucleic acid comprises an expression construct encoding an inhibitory nucleic acid that inhibits the expression or activity of ATXN2, wherein the inhibitory nucleic acid comprises a guide strand sequence comprising or consisting of a nucleic acid sequence that is at least 60%, 70%, 75%, 80%, 85%, 90%, 95%, 97%, 99%, or 100% identical to any one of SEQ ID NOS:1908-2007. In some embodiments, the isolated nucleic acid comprises an expression construct encoding an inhibitory nucleic acid that inhibits the expression or activity of ATXN2, wherein the inhibitory nucleic acid comprises a guide strand sequence comprising or consisting of at least 15, 16, 17, 18, 19, 20, 21, or 22 contiguous nucleotides of a sequence of any one of SEQ ID NOS:1908-2007, preferably wherein the guide strand sequence retains positions 2-7 (“seed sequence”) of the selected SEQ ID NO. In some embodiments, the isolated nucleic acid comprises an expression construct encoding an inhibitory nucleic acid that inhibits the expression or activity of ATXN2, wherein the inhibitory nucleic acid comprises a guide strand sequence comprising or consisting of a sequence of any one of SEQ ID NOS: 1908-2007, wherein 1, 2, 3, or 4 nucleotides at positions 19-22 differ from the selected SEQ ID NO (variant nucleotide(s)), such that the guide strand sequence is no longer complementary to the ATXN2 target sequence at the variant nucleotide(s).

In some embodiments, the isolated nucleic acid comprises an expression construct encoding an inhibitory nucleic acid that inhibits the expression or activity of ATXN2, wherein the inhibitory nucleic acid comprises a guide strand sequence of Table 24. In some embodiments, the inhibitory nucleic acid comprises a guide strand sequence comprising or consisting of the nucleic acid sequence set forth in any one of SEQ ID NOS:100, 112, 166, 202, 246, 306, 308, 314, 1180, 1185, 1196, 1200, 1211, 1213, 1215, 1216, 1224, 1811-1822, 1824-1827, 2015, 2065, 2083, 2152, 2203, and 2209. In some embodiments, the isolated nucleic acid comprises an expression construct encoding an inhibitory nucleic acid that inhibits the expression or activity of ATXN2, wherein the inhibitory nucleic acid comprises a guide strand sequence comprising or consisting of the nucleic acid sequence set forth in any one of SEQ ID NOS:100, 112, 166, 202, 246, 306, 308, 314, 1180, 1185, 1196, 1200, 1211, 1213, 1215, 1216, 1224, 1811-1822, 1824-1827, 2015, 2065, 2083, 2152, 2203, and 2209 with at least 1, 2, 3, 4, or 5 mismatches to the target ATXN2 mRNA sequence. In some embodiments, the isolated nucleic acid comprises an expression construct encoding an inhibitory nucleic acid that inhibits the expression or activity of ATXN2, wherein the inhibitory nucleic acid comprises a guide strand sequence comprising or consisting of a nucleic acid sequence that is at least 60%, 70%, 75%, 80%, 85%, 90%, 95%, 97%, 99%, or 100% identical to any one of SEQ ID NOS:100, 112, 166, 202, 246, 306, 308, 314, 1180, 1185, 1196, 1200, 1211, 1213, 1215, 1216, 1224, 1811-1822, 1824-1827, 2015, 2065, 2083, 2152, 2203, and 2209. In some embodiments, the isolated nucleic acid comprises an expression construct encoding an inhibitory nucleic acid that inhibits the expression or activity of ATXN2, wherein the inhibitory nucleic acid comprises a guide strand sequence comprising or consisting of at least 15, 16, 17, 18, 19, 20, 21, or 22 contiguous nucleotides of a sequence of any one of SEQ ID NOS:100, 112, 166, 202, 246, 306, 308, 314, 1180, 1185, 1196, 1200, 1211, 1213, 1215, 1216, 1224, 1811-1822, 1824-1827, 2015, 2065, 2083, 2152, 2203, and 2209, preferably wherein the guide strand sequence retains positions 2-7 (“seed sequence”) of the selected SEQ ID NO. In some embodiments, the isolated nucleic acid comprises an expression construct encoding an inhibitory nucleic acid that inhibits the expression or activity of ATXN2, wherein the inhibitory nucleic acid comprises a guide strand sequence comprising or consisting of a sequence of any one of SEQ ID NOS:100, 112, 166, 202, 246, 306, 308, 314, 1180, 1185, 1196, 1200, 1211, 1213, 1215, 1216, 1224, 1811-1822, 1824-1827, 2015, 2065, 2083, 2152, 2203, and 2209, wherein 1, 2, 3, or 4 nucleotides at positions 19-22 differ from the selected SEQ ID NO (variant nucleotide(s)), such that the guide strand sequence is no longer complementary to the ATXN2 target sequence at the variant nucleotide(s).

In some embodiments, the isolated nucleic acid comprises an expression construct encoding an inhibitory nucleic acid that inhibits the expression or activity of ATXN2, wherein the inhibitory nucleic acid comprises a guide strand sequence of Table 25. In some embodiments, the inhibitory nucleic acid comprises a guide strand sequence comprising or consisting of the nucleic acid sequence set forth in any one of SEQ ID NOS:1185, 1816, 1213, 1819, 2083, 1215, 1216, 1811, and 314. In some embodiments, the isolated nucleic acid comprises an expression construct encoding an inhibitory nucleic acid that inhibits the expression or activity of ATXN2, wherein the inhibitory nucleic acid comprises a guide strand sequence comprising or consisting of the nucleic acid sequence set forth in any one of SEQ ID NOS:1185, 1816, 1213, 1819, 2083, 1215, 1216, 1811, and 314 with at least 1, 2, 3, 4, or 5 mismatches to the target ATXN2 mRNA sequence. In some embodiments, the isolated nucleic acid comprises an expression construct encoding an inhibitory nucleic acid that inhibits the expression or activity of ATXN2, wherein the inhibitory nucleic acid comprises a guide strand sequence comprising or consisting of a nucleic acid sequence that is at least 60%, 70%, 75%, 80%, 85%, 90%, 95%, 97%, 99%, or 100% identical to any one of SEQ ID NOS:1185, 1816, 1213, 1819, 2083, 1215, 1216, 1811, and 314. In some embodiments, the isolated nucleic acid comprises an expression construct encoding an inhibitory nucleic acid that inhibits the expression or activity of ATXN2, wherein the inhibitory nucleic acid comprises a guide strand sequence comprising or consisting of at least 15, 16, 17, 18, 19, 20, 21, or 22 contiguous nucleotides of a sequence of any one of SEQ ID NOS:1185, 1816, 1213, 1819, 2083, 1215, 1216, 1811, and 314, preferably wherein the guide strand sequence retains positions 2-7 (“seed sequence”) of the selected SEQ ID NO. In some embodiments, the isolated nucleic acid comprises an expression construct encoding an inhibitory nucleic acid that inhibits the expression or activity of ATXN2, wherein the inhibitory nucleic acid comprises a guide strand sequence comprising or consisting of a sequence of any one of SEQ ID NOS:1185, 1816, 1213, 1819, 2083, 1215, 1216, 1811, and 314, wherein 1, 2, 3, or 4 nucleotides at positions 19-22 differ from the selected SEQ ID NO (variant nucleotide(s)), such that the guide strand sequence is no longer complementary to the ATXN2 target sequence at the variant nucleotide(s).

In some embodiments, the isolated nucleic acid comprises an expression construct encoding an inhibitory nucleic acid that inhibits the expression or activity of ATXN2, wherein the inhibitory nucleic acid comprises a guide strand sequence comprising or consisting of the nucleic acid sequence set forth in any one of SEQ ID NOS:1185, 1816, 1213, and 1811. In some embodiments, the isolated nucleic acid comprises an expression construct encoding an inhibitory nucleic acid that inhibits the expression or activity of ATXN2, wherein the inhibitory nucleic acid comprises a guide strand sequence comprising or consisting of the nucleic acid sequence set forth in any one of SEQ ID NOS:1185, 1816, 1213, and 1811, with at least 1, 2, 3, 4, or 5 mismatches to the target ATXN2 mRNA sequence. In some embodiments, the isolated nucleic acid comprises an expression construct encoding an inhibitory nucleic acid that inhibits the expression or activity of ATXN2, wherein the inhibitory nucleic acid comprises a guide strand sequence comprising or consisting of a nucleic acid sequence that is at least 60%, 70%, 75%, 80%, 85%, 90%, 95%, 97%, 99%, or 100% identical to any one of SEQ ID NOS:1185, 1816, 1213, and 1811. In some embodiments, the isolated nucleic acid comprises an expression construct encoding an inhibitory nucleic acid that inhibits the expression or activity of ATXN2, wherein the inhibitory nucleic acid comprises a guide strand sequence comprising or consisting of at least 15, 16, 17, 18, 19, 20, 21, or 22 contiguous nucleotides of a sequence of any one of SEQ ID NOS:1185, 1816, 1213, and 1811, preferably wherein the guide strand sequence retains positions 2-7 (“seed sequence”) of the selected SEQ ID NO. In some embodiments, the isolated nucleic acid comprises an expression construct encoding an inhibitory nucleic acid that inhibits the expression or activity of ATXN2, wherein the inhibitory nucleic acid comprises a guide strand sequence comprising or consisting of a sequence of any one of SEQ ID NOS:1185, 1816, 1213, and 1811, wherein 1, 2, 3, or 4 nucleotides at positions 19-22 differ from the selected SEQ ID NO (variant nucleotide(s)), such that the guide strand sequence is no longer complementary to the ATXN2 target sequence at the variant nucleotide(s).

In some embodiments, the isolated nucleic acid comprises an expression construct encoding a siRNA duplex that targets ATXN2 mRNA to interfere with ATXN2 expression by mRNA degradation or translational inhibition. In some embodiments, the guide strand of the siRNA duplex may be about 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides in length or 18-30, 18-29, 18-28, 18-27, 18-26, 18-25, 18-24, 18-23, 18-22, 18-20, 19-30, 19-29, 19-28, 19-27, 19-26, 19-25, 19-24, 19-23, 19-22, 19-21, 20-30, 20-29, 20-28, 20-27, 20-26, 20-25, 20-24, 20-23, 20-22, 21-30, 21-29, 21-28, 21-27, 21-26, 21-25, 21-24, 21-23, 22-30, 22-29, 22-28, 22-27, 22-26, 22-24, 23-30, 23-29, 23-28, 23-27, 23-26, 23-25, 24-30, 24-29, 24-28, 24-27, 24-26, 25-30, 25-29, 25-28, 25-27, 26-30, 26-29, 26-28, 27-30, 27-29, 28-30 nucleotides in length. In some embodiments, the passenger strand of the siRNA duplex may be about 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides in length or 18-30, 18-29, 18-28, 18-27, 18-26, 18-25, 18-24, 18-23, 18-22, 18-20, 19-30, 19-29, 19-28, 19-27, 19-26, 19-25, 19-24, 19-23, 19-22, 19-21, 20-30, 20-29, 20-28, 20-27, 20-26, 20-25, 20-24, 20-23, 20-22, 21-30, 21-29, 21-28, 21-27, 21-26, 21-25, 21-24, 21-23, 22-30, 22-29, 22-28, 22-27, 22-26, 22-24, 23-30, 23-29, 23-28, 23-27, 23-26, 23-25, 24-30, 24-29, 24-28, 24-27, 24-26, 25-30, 25-29, 25-28, 25-27, 26-30, 26-29, 26-28, 27-30, 27-29, 28-30 nucleotides in length. In some embodiments, the siRNA duplex contains 2 or 3 nucleotide 3′ overhangs on each strand. In some embodiments, the 3′ overhangs are complementary to the ATXN2 transcript. In some embodiments, the guide strand and passenger strand of the siRNA duplex are at least 60%, 65%, 70%, 75%, 80%, 85%, 90%, 95%, 97%, or 100% complementary to each other, not including any nucleotides in overhang(s).

In some embodiments, the isolated nucleic acid comprises an expression construct encoding a siRNA duplex that comprises a guide strand sequence comprising or consisting of the nucleic acid sequence set forth in any one of the guide sequences of Tables 1, 3, 9, 11, 12, 13, 19, 23, and 24, e.g., any one of SEQ ID NOS: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 1176-1288, 1811-1827, 2015, 2065, 2083, 2152, 2203, and 2209.

In some embodiments, the isolated nucleic acid comprises an expression construct encoding a siRNA duplex that comprises a guide strand sequence comprising or consisting of the nucleic acid sequence set forth in any one of the guide sequences of Tables 1, 3, 9, 11, 12, 13, 19, 23, and 24, e.g., any one of SEQ ID NOS: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 1176-1288, 1811-1827, 2015, 2065, 2083, 2152, 2203, and 2209, with at least 1, 2, 3, 4, or 5 mismatches to the target ATXN2 mRNA sequence.

In some embodiments, the isolated nucleic acid comprises an expression construct encoding a siRNA duplex that comprises a guide strand sequence sequence comprising of consisting of a sequence that at least 60%, 70%, 75%, 80%, 85%, 90%, 95%, 97%, 99%, or 100% identical to any one of the guide sequences of Tables 1, 3, 9, 11, 12, 13, 19, 23, and 24, e.g., any one of SEQ ID NOS: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 1176-1288, 1811-1827, 2015, 2065, 2083, 2152, 2203, and 2209.

In some embodiments, the isolated nucleic acid comprises an expression construct encoding a siRNA duplex that comprises a guide strand sequence comprising or consisting of at least 15, 16, 17, 18, 19, 20, 21, or 22 contiguous nucleotides of a sequence of any one of the guide sequences of Tables 1, 3, 9, 11, 12, 13, 19, 23, and 24, e.g., any one of SEQ ID NOS: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 1176-1288, 1811-1827, 2015, 2065, 2083, 2152, 2203, and 2209, preferably wherein the guide strand sequence retains positions 2-7 (“seed sequence”) of the selected SEQ ID NO.

In some embodiments, the isolated nucleic acid comprises an expression construct encoding a siRNA duplex that comprises a guide strand sequence comprising or consisting of any one of the guide sequences of Tables 1, 3, 9, 11, 12, 13, 19, 23, and 24, e.g., any one of SEQ ID NOS: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 1176-1288, 1811-1827, 2015, 2065, 2083, 2152, 2203, and 2209, wherein 1, 2, 3, or 4 nucleotides at positions 19-22 differ from the selected SEQ ID NO (variant nucleotide(s)), such that the guide strand sequence is no longer complementary to the ATXN2 target sequence at the variant nucleotide(s).

In some embodiments, the isolated nucleic acid comprises an expression construct encoding a siRNA duplex that comprises a guide strand sequence of Table 12. In some embodiments, the isolated nucleic acid comprises an expression construct encoding a siRNA duplex that comprises a guide strand sequence comprising or consisting of the nucleic acid sequence set forth in any one of SEQ ID NOS: 12, 14, 40, 60, 100, 104, 108, 112, 124, 126, 128, 166, 198, 220, 242, 302, 306, 308, 330, 336, and 362. In some embodiments, the isolated nucleic acid comprises an expression construct encoding a siRNA duplex that comprises a guide strand sequence comprising or consisting of the nucleic acid sequence set forth in any one of SEQ ID NOS: 12, 14, 40, 60, 100, 104, 108, 112, 124, 126, 128, 166, 198, 220, 242, 302, 306, 308, 330, 336, and 362 with at least 1, 2, 3, 4, or 5 mismatches to the target ATXN2 mRNA sequence. In some embodiments, the siRNA duplex comprises a guide strand sequence comprising or consisting of a nucleic acid sequence that is at least 60%, 70%, 75%, 80%, 85%, 90%, 95%, 97%, 99%, or 100% identical to any one of SEQ ID NOS: 12, 14, 40, 60, 100, 104, 108, 112, 124, 126, 128, 166, 198, 220, 242, 302, 306, 308, 330, 336, and 362. In some embodiments, the isolated nucleic acid comprises an expression construct encoding a siRNA duplex that comprises a guide strand sequence comprising or consisting of at least 15, 16, 17, 18, 19, 20, 21, or 22 contiguous nucleotides of a sequence of any one of SEQ ID NOS: 12, 14, 40, 60, 100, 104, 108, 112, 124, 126, 128, 166, 198, 220, 242, 302, 306, 308, 330, 336, and 362, preferably wherein the guide strand sequence retains positions 2-7 (“seed sequence”) of the selected SEQ ID NO. In some embodiments, the isolated nucleic acid comprises an expression construct encoding a siRNA duplex that comprises a guide strand sequence comprising or consisting of a sequence of any one of SEQ ID NOS: 12, 14, 40, 60, 100, 104, 108, 112, 124, 126, 128, 166, 198, 220, 242, 302, 306, 308, 330, 336, and 362, wherein 1, 2, 3, or 4 nucleotides at positions 19-22 differ from the selected SEQ ID NO (variant nucleotide(s)), such that the guide strand sequence is no longer complementary to the ATXN2 target sequence at the variant nucleotide(s).

In some embodiments, the isolated nucleic acid comprises an expression construct encoding a siRNA duplex that comprises a guide strand sequence of Table 13. In some embodiments, the isolated nucleic acid comprises an expression construct encoding a siRNA duplex that comprises a guide strand sequence comprising or consisting of the nucleic acid sequence set forth in any one of SEQ ID NOS: 14, 40, 100, 108, 112, 128, 166, 198, 242, 308, 336, and 362. In some embodiments, the isolated nucleic acid comprises an expression construct encoding a siRNA duplex that comprises a guide strand sequence comprising or consisting of the nucleic acid sequence set forth in any one of SEQ ID NOS: 14, 40, 100, 108, 112, 128, 166, 198, 242, 308, 336, and 362 with at least 1, 2, 3, 4, or 5 mismatches to the target ATXN2 mRNA sequence. In some embodiments, the isolated nucleic acid comprises an expression construct encoding a siRNA duplex that comprises a guide strand sequence comprising or consisting of a nucleic acid sequence that is at least 60%, 70%, 75%, 80%, 85%, 90%, 95%, 97%, 99%, or 100% identical to any one of SEQ ID NOS: 14, 40, 100, 108, 112, 128, 166, 198, 242, 308, 336, and 362. In some embodiments, the isolated nucleic acid comprises an expression construct encoding a siRNA duplex that comprises a guide strand sequence comprising a guide strand sequence comprising or consisting of at least 15, 16, 17, 18, 19, 20, 21, or 22 contiguous nucleotides of a sequence of any one of SEQ ID NOS: 14, 40, 100, 108, 112, 128, 166, 198, 242, 308, 336, and 362, preferably wherein the guide strand sequence retains positions 2-7 (“seed sequence”) of the selected SEQ ID NO. In some embodiments, the isolated nucleic acid comprises an expression construct encoding a siRNA duplex that comprises a guide strand sequence comprising or consisting of a sequence of any one of SEQ ID NOS: 14, 40, 100, 108, 112, 128, 166, 198, 242, 308, 336, and 362, wherein 1, 2, 3, or 4 nucleotides at positions 19-22 differ from the selected SEQ ID NO (variant nucleotide(s)), such that the guide strand sequence is no longer complementary to the ATXN2 target sequence at the variant nucleotide(s).

In some embodiments, the isolated nucleic acid comprises an expression construct encoding a siRNA duplex that comprises a guide strand sequence of Table 19. In some embodiments, the isolated nucleic acid comprises an expression construct encoding a siRNA duplex that comprises a guide strand sequence comprising or consisting of the nucleic acid sequence set forth in any one of SEQ ID NOS:1176-1288, 40, 108, and 166. In some embodiments, the isolated nucleic acid comprises an expression construct encoding a siRNA duplex that comprises a guide strand sequence comprising or consisting of the nucleic acid sequence set forth in any one of SEQ ID NOS:1176-1288, 40, 108, and 166 with at least 1, 2, 3, 4, or 5 mismatches to the target ATXN2 mRNA sequence. In some embodiments, the isolated nucleic acid comprises an expression construct encoding a siRNA duplex that comprises a guide strand sequence comprising or consisting of a nucleic acid sequence that is at least 60%, 70%, 75%, 80%, 85%, 90%, 95%, 97%, 99%, or 100% identical to any one of SEQ ID NOS:1176-1288, 40, 108, and 166. In some embodiments, the isolated nucleic acid comprises an expression construct encoding a siRNA duplex that comprises a guide strand sequence comprising a guide strand sequence comprising or consisting of at least 15, 16, 17, 18, 19, 20, 21, or 22 contiguous nucleotides of a sequence of any one of SEQ ID NOS:1176-1288, 40, 108, and 166, preferably wherein the guide strand sequence retains positions 2-7 (“seed sequence”) of the selected SEQ ID NO. In some embodiments, the isolated nucleic acid comprises an expression construct encoding a siRNA duplex that comprises a guide strand sequence comprising or consisting of a sequence of any one of SEQ ID NOS:1176-1288, 40, 108, and 166, wherein 1, 2, 3, or 4 nucleotides at positions 19-22 differ from the selected SEQ ID NO (variant nucleotide(s)), such that the guide strand sequence is no longer complementary to the ATXN2 target sequence at the variant nucleotide(s).

In some embodiments, the isolated nucleic acid comprises an expression construct encoding a siRNA duplex that comprises a guide strand sequence of Table 23. In some embodiments, the isolated nucleic acid comprises an expression construct encoding a siRNA duplex that comprises a guide strand sequence comprising or consisting of the nucleic acid sequence set forth in any one of SEQ ID NOS:1908-2007. In some embodiments, the isolated nucleic acid comprises an expression construct encoding a siRNA duplex that comprises a guide strand sequence comprising or consisting of the nucleic acid sequence set forth in any one of SEQ ID NOS:1908-2007 with at least 1, 2, 3, 4, or 5 mismatches to the target ATXN2 mRNA sequence. In some embodiments, the isolated nucleic acid comprises an expression construct encoding a siRNA duplex that comprises a guide strand sequence comprising or consisting of a nucleic acid sequence that is at least 60%, 70%, 75%, 80%, 85%, 90%, 95%, 97%, 99%, or 100% identical to any one of SEQ ID NOS:1908-2007. In some embodiments, the isolated nucleic acid comprises an expression construct encoding a siRNA duplex that comprises a guide strand sequence comprising a guide strand sequence comprising or consisting of at least 15, 16, 17, 18, 19, 20, 21, or 22 contiguous nucleotides of a sequence of any one of SEQ ID NOS:1908-2007, preferably wherein the guide strand sequence retains positions 2-7 (“seed sequence”) of the selected SEQ ID NO. In some embodiments, the isolated nucleic acid comprises an expression construct encoding a siRNA duplex that comprises a guide strand sequence comprising or consisting of a sequence of any one of SEQ ID NOS:1908-2007, wherein 1, 2, 3, or 4 nucleotides at positions 19-22 differ from the selected SEQ ID NO (variant nucleotide(s)), such that the guide strand sequence is no longer complementary to the ATXN2 target sequence at the variant nucleotide(s).

In some embodiments, the isolated nucleic acid comprises an expression construct encoding a siRNA duplex that comprises a guide strand sequence of Table 24. In some embodiments, the isolated nucleic acid comprises an expression construct encoding a siRNA duplex that comprises a guide strand sequence comprising or consisting of the nucleic acid sequence set forth in any one of SEQ ID NOS:100, 112, 166, 202, 246, 306, 308, 314, 1180, 1185, 1196, 1200, 1211, 1213, 1215, 1216, 1224, 1811-1822, 1824-1827, 2015, 2065, 2083, 2152, 2203, and 2209. In some embodiments, the isolated nucleic acid comprises an expression construct encoding a siRNA duplex that comprises a guide strand sequence comprising or consisting of the nucleic acid sequence set forth in any one of SEQ ID NOS:100, 112, 166, 202, 246, 306, 308, 314, 1180, 1185, 1196, 1200, 1211, 1213, 1215, 1216, 1224, 1811-1822, 1824-1827, 2015, 2065, 2083, 2152, 2203, and 2209, with at least 1, 2, 3, 4, or 5 mismatches to the target ATXN2 mRNA sequence. In some embodiments, the isolated nucleic acid comprises an expression construct encoding a siRNA duplex that comprises a guide strand sequence comprising or consisting of a nucleic acid sequence that is at least 60%, 70%, 75%, 80%, 85%, 90%, 95%, 97%, 99%, or 100% identical to any one of SEQ ID NOS:100, 112, 166, 202, 246, 306, 308, 314, 1180, 1185, 1196, 1200, 1211, 1213, 1215, 1216, 1224, 1811-1822, 1824-1827, 2015, 2065, 2083, 2152, 2203, and 2209. In some embodiments, the isolated nucleic acid comprises an expression construct encoding a siRNA duplex that comprises a guide strand sequence comprising a guide strand sequence comprising or consisting of at least 15, 16, 17, 18, 19, 20, 21, or 22 contiguous nucleotides of a sequence of any one of SEQ ID NOS:100, 112, 166, 202, 246, 306, 308, 314, 1180, 1185, 1196, 1200, 1211, 1213, 1215, 1216, 1224, 1811-1822, 1824-1827, 2015, 2065, 2083, 2152, 2203, and 2209, preferably wherein the guide strand sequence retains positions 2-7 (“seed sequence”) of the selected SEQ ID NO. In some embodiments, the isolated nucleic acid comprises an expression construct encoding a siRNA duplex that comprises a guide strand sequence comprising or consisting of a sequence of any one of SEQ ID NOS:100, 112, 166, 202, 246, 306, 308, 314, 1180, 1185, 1196, 1200, 1211, 1213, 1215, 1216, 1224, 1811-1822, 1824-1827, 2015, 2065, 2083, 2152, 2203, and 2209, wherein 1, 2, 3, or 4 nucleotides at positions 19-22 differ from the selected SEQ ID NO (variant nucleotide(s)), such that the guide strand sequence is no longer complementary to the ATXN2 target sequence at the variant nucleotide(s).

In some embodiments, the isolated nucleic acid comprises an expression construct encoding a siRNA duplex that comprises a guide strand sequence of Table 25. In some embodiments, the isolated nucleic acid comprises an expression construct encoding a siRNA duplex that comprises a guide strand sequence comprising or consisting of the nucleic acid sequence set forth in any one of SEQ ID NOS:1185, 1816, 1213, 1819, 2083, 1215, 1216, 1811, and 314. In some embodiments, the isolated nucleic acid comprises an expression construct encoding a siRNA duplex that comprises a guide strand sequence comprising or consisting of the nucleic acid sequence set forth in any one of SEQ ID NOS:1185, 1816, 1213, 1819, 2083, 1215, 1216, 1811, and 314, with at least 1, 2, 3, 4, or 5 mismatches to the target ATXN2 mRNA sequence. In some embodiments, the isolated nucleic acid comprises an expression construct encoding a siRNA duplex that comprises a guide strand sequence comprising or consisting of a nucleic acid sequence that is at least 60%, 70%, 75%, 80%, 85%, 90%, 95%, 97%, 99%, or 100% identical to any one of SEQ ID NOS:1185, 1816, 1213, 1819, 2083, 1215, 1216, 1811, and 314. In some embodiments, the isolated nucleic acid comprises an expression construct encoding a siRNA duplex that comprises a guide strand sequence comprising a guide strand sequence comprising or consisting of at least 15, 16, 17, 18, 19, 20, 21, or 22 contiguous nucleotides of a sequence of any one of SEQ ID NOS:1185, 1816, 1213, 1819, 2083, 1215, 1216, 1811, and 314, preferably wherein the guide strand sequence retains positions 2-7 (“seed sequence”) of the selected SEQ ID NO. In some embodiments, the isolated nucleic acid comprises an expression construct encoding a siRNA duplex that comprises a guide strand sequence comprising or consisting of a sequence of any one of SEQ ID NOS:1185, 1816, 1213, 1819, 2083, 1215, 1216, 1811, and 314, wherein 1, 2, 3, or 4 nucleotides at positions 19-22 differ from the selected SEQ ID NO (variant nucleotide(s)), such that the guide strand sequence is no longer complementary to the ATXN2 target sequence at the variant nucleotide(s).

In some embodiments the isolated nucleic acid comprises an expression construct encoding a siRNA duplex comprising a guide strand sequence and passenger strand sequence of any one of siRNA duplexes provided in Tables 1, 19, 23, and 24. In some embodiments the isolated nucleic acid comprises an expression construct encoding a siRNA duplex comprising a guide strand sequence and passenger strand sequence, comprising or consisting of any one of: SEQ ID NOS:12 and 11; SEQ ID NOS: 14 and 13; SEQ ID NOS: 40 and 39; SEQ ID NOS: 60 and 59; SEQ ID NOS: 100 and 99; SEQ ID NOS: 104 and 103; SEQ ID NOS: 108 and 107; SEQ ID NOS: 112 and 111; SEQ ID NOS: 124 and 123; SEQ ID NOS: 126 and 125; SEQ ID NOS: 128 and 127; SEQ ID NOS: 166 and 165; SEQ ID NOS: 198 and 197; SEQ ID NOS: 220 and 219; SEQ ID NOS: 242 and 241; SEQ ID NOS: 302 and 301; SEQ ID NOS: 306 and 305; SEQ ID NOS: 308 and 307; SEQ ID NOS: 330 and 320; SEQ ID NOS: 336 and 335; and SEQ ID NOS: 362 and 361. In some embodiments the isolated nucleic acid comprises an expression construct encoding a siRNA duplex comprising a guide strand sequence and passenger strand sequence comprising or consisting of any one of: SEQ ID NOS:14 and 13; SEQ ID NOS: 40 and 39; SEQ ID NOS: 100 and 99; SEQ ID NOS: 108 and 107: SEQ ID NOS: 112 and 11; SEQ ID NOS: 128 and 127; SEQ ID NOS: 166 and 165; SEQ ID NOS: 198 and 197; SEQ ID NOS: 242 and 241; SEQ ID NOS: 308 and 307; SEQ ID NOS: 336 and 335; and SEQ ID NOS: 362 and 361.

In some embodiments, the isolated nucleic acid comprises an expression construct encoding a shRNA comprising a guide strand and passenger strand of a siRNA duplex as provided herein, linked by a short spacer sequence, i.e., loop. In some embodiments, loop sequence is 4, 5, 6, 7, 8, 9, or 10 nucleotides in length or 4-10, 4-9, 4-8, 4-7, 4-6, 5-10, 5-9, 5-8, 5-7, 6-9, 6-8, 7-10, 7-9, or 8-10 nucleotides in length.

In some embodiments, the isolated nucleic acid comprises an expression construct encoding a miRNA, such as a pri-miRNA, a pre-mRNA, an artificial miRNA, or a mature miRNA, wherein the miRNA comprises a guide strand sequence comprising or consisting of the nucleic acid sequence set forth in any one of the guide sequences of Tables 1, 3, 9, 11, 12, 13, 19, 23, 24, and 25, e.g., any one of SEQ ID NOS: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 1176-1288, 1811-1827, 2015, 2065, 2083, 2152, 2203, and 2209.

In some embodiments, the isolated nucleic acid comprises an expression construct encoding a miRNA, such as a pri-miRNA, a pre-mRNA, an artificial miRNA, or a mature miRNA, wherein the miRNA comprises a guide strand sequence comprising or consisting of the nucleic acid sequence set forth in any one of the guide sequences of Tables 1, 3, 9, 11, 12, 13, 19, 24, and 25, e.g., any one of SEQ ID NOS: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 1176-1288, 1811-1827, 2015, 2065, 2083, 2152, 2203, and 2209, with at least 1, 2, 3, 4, or 5 mismatches to the target ATXN2 mRNA sequence.

In some embodiments, the isolated nucleic acid comprises an expression construct encoding a miRNA, such as a pri-miRNA, a pre-mRNA, an artificial miRNA, or a mature miRNA, wherein the miRNA comprises a guide strand sequence comprising or consisting of a sequence that is at least 60%, 70%, 75%, 80%, 85%, 90%, 95%, 97%, 99%, or 100% identical to any one of the guide sequences of Tables 1, 3, 9, 11, 12, 13, 19, 23, 24, and 25, e.g., any one of SEQ ID NOS: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 1176-1288, 1811-1827, 2015, 2065, 2083, 2152, 2203, and 2209.

In some embodiments, the isolated nucleic acid comprises an expression construct encoding a miRNA, such as a pri-miRNA, a pre-mRNA, an artificial miRNA, or a mature miRNA, wherein the miRNA comprises a guide strand sequence comprising or consisting of at least 15, 16, 17, 18, 19, 20, 21, or 22 contiguous nucleotides of a sequence of any one of the guide sequences of Tables 1, 3, 9, 11, 12, 13, 19, 23, 24, and 25, e.g., any one of SEQ ID NOS: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 1176-1288, 1811-1827, 2015, 2065, 2083, 2152, 2203, and 2209, preferably wherein the guide strand sequence retains positions 2-7 (“seed sequence”) of the selected SEQ ID NO.

In some embodiments, the isolated nucleic acid comprises an expression construct encoding a miRNA, such as a pri-miRNA, a pre-mRNA, an artificial miRNA, or a mature miRNA, wherein the miRNA comprises a guide strand sequence comprising or consisting of a nucleic acid sequence of any one of the guide sequences of Tables 1, 3, 9, 11, 12, 13, 19, 23, 24, and 25, e.g., any one of SEQ ID NOS: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 1176-1288, 1811-1827, 2015, 2065, 2083, 2152, 2203, and 2209, wherein 1, 2, 3, or 4 nucleotides at positions 19-22 differ from the selected SEQ ID NO (variant nucleotide(s)) such that the guide strand sequence is no longer complementary to the ATXN2 target sequence at the variant nucleotide(s).

In some embodiments, the isolated nucleic acid comprises an expression construct encoding a miRNA, such as a pri-miRNA, a pre-mRNA, an artificial miRNA, or a mature miRNA comprising a guide strand sequence of Table 12. In some embodiments, the isolated nucleic acid comprises an expression construct encoding a miRNA, such as a pri-miRNA, a pre-mRNA, an artificial miRNA, or a mature miRNA comprising a guide strand sequence comprising or consisting of the nucleic acid sequence set forth in any one of SEQ ID NOS: 12, 14, 40, 60, 100, 104, 108, 112, 124, 126, 128, 166, 198, 220, 242, 302, 306, 308, 330, 336, and 362. In some embodiments, the isolated nucleic acid comprises an expression construct encoding a miRNA, such as a pri-miRNA, a pre-mRNA, an artificial miRNA, or a mature miRNA, wherein the miRNA comprises a guide strand sequence comprising or consisting of the nucleic acid sequence set forth in any one of SEQ ID NOS: 12, 14, 40, 60, 100, 104, 108, 112, 124, 126, 128, 166, 198, 220, 242, 302, 306, 308, 330, 336, and 362, with at least 1, 2, 3, 4, or 5 mismatches to the target ATXN2 mRNA sequence. In some embodiments, the isolated nucleic acid comprises an expression construct encoding a miRNA, such as a pri-miRNA, a pre-mRNA, an artificial miRNA, or a mature miRNA, wherein the miRNA comprises a guide strand sequence comprising or consisting of a nucleic acid sequence that is at least 60%, 70%, 75%, 80%, 85%, 90%, 95%, 97%, 99%, or 100% identical to any one of SEQ ID NOS: 12, 14, 40, 60, 100, 104, 108, 112, 124, 126, 128, 166, 198, 220, 242, 302, 306, 308, 330, 336, and 362. In some embodiments, the isolated nucleic acid comprises an expression construct encoding a miRNA, such as a pri-miRNA, a pre-mRNA, an artificial miRNA, or a mature miRNA, wherein the miRNA comprises a guide strand sequence comprising or consisting of at least 15, 16, 17, 18, 19, 20, 21, or 22 contiguous nucleotides of a sequence of any one of SEQ ID NOS: 12, 14, 40, 60, 100, 104, 108, 112, 124, 126, 128, 166, 198, 220, 242, 302, 306, 308, 330, 336, and 362, preferably wherein the guide strand sequence retains positions 2-7 (“seed sequence”) of the selected SEQ ID NO. In some embodiments, the isolated nucleic acid comprises an expression construct encoding a miRNA, such as a pri-miRNA, a pre-mRNA, an artificial miRNA, or a mature miRNA, wherein the miRNA comprises a guide strand sequence comprising or consisting of a sequence of any one of SEQ ID NOS: 12, 14, 40, 60, 100, 104, 108, 112, 124, 126, 128, 166, 198, 220, 242, 302, 306, 308, 330, 336, and 362, wherein 1, 2, 3, or 4 nucleotides at positions 19-22 differ from the selected SEQ ID NO (variant nucleotide(s)) such that the guide strand sequence is no longer complementary to the ATXN2 target sequence at the variant nucleotide(s).

In some embodiments, the isolated nucleic acid comprises an expression construct encoding a miRNA, such as a pri-miRNA, a pre-mRNA, an artificial miRNA, or a mature miRNA comprising a guide strand sequence of Table 13. In some embodiments, the isolated nucleic acid comprises an expression construct encoding a miRNA, such as a pri-miRNA, a pre-mRNA, an artificial miRNA, or a mature miRNA, comprising a guide strand sequence comprising or consisting of the nucleic acid sequence set forth in any one of SEQ ID NOS: 14, 40, 100, 108, 112, 128, 166, 198, 242, 308, 336, and 362. In some embodiments, the isolated nucleic acid comprises an expression construct encoding a miRNA, such as a pri-miRNA, a pre-mRNA, an artificial miRNA, or a mature miRNA, wherein the miRNA comprises a guide strand sequence comprising or consisting of the nucleic acid sequence set forth in any one of SEQ ID NOS: 14, 40, 100, 108, 112, 128, 166, 198, 242, 308, 336, and 362, with at least 1, 2, 3, 4, or 5 mismatches to the target ATXN2 mRNA sequence. In some embodiments, the miRNA is a pri-miRNA, a pre-mRNA, an artificial miRNA, or a mature miRNA wherein the miRNA comprises a guide strand sequence comprising or consisting of a nucleic acid sequence that is at least 60%, 70%, 75%, 80%, 85%, 90%, 95%, 97%, 99%, or 100% identical to any one of SEQ ID NOS: 14, 40, 100, 108, 112, 128, 166, 198, 242, 308, 336, and 362. In some embodiments, the isolated nucleic acid comprises an expression construct encoding a miRNA, such as a pri-miRNA, a pre-mRNA, an artificial miRNA, or a mature miRNA, wherein the miRNA comprises a guide strand sequence comprising or consisting of at least 15, 16, 17, 18, 19, 20, 21, or 22 contiguous nucleotides of a sequence of any one of SEQ ID NOS: 14, 40, 100, 108, 112, 128, 166, 198, 242, 308, 336, and 362, preferably wherein the guide strand sequence retains positions 2-7 (“seed sequence”) of the selected SEQ ID NO. In some embodiments, the isolated nucleic acid comprises an expression construct encoding a miRNA, such as a pri-miRNA, a pre-mRNA, an artificial miRNA, or a mature miRNA, wherein the miRNA comprises a guide strand sequence comprising or consisting of a sequence of any one of SEQ ID NOS: 14, 40, 100, 108, 112, 128, 166, 198, 242, 308, 336, and 362, wherein 1, 2, 3, or 4 nucleotides at positions 19-22 differ from the selected SEQ ID NO (variant nucleotide(s)), such that the guide strand sequence is no longer complementary to the ATXN2 target sequence at the variant nucleotide(s).

In some embodiments, the isolated nucleic acid comprises an expression construct encoding a miRNA, such as a pri-miRNA, a pre-mRNA, an artificial miRNA, or a mature miRNA comprising a guide strand sequence of Table 19. In some embodiments, the isolated nucleic acid comprises an expression construct encoding a miRNA, such as a pri-miRNA, a pre-mRNA, an artificial miRNA, or a mature miRNA, comprising a guide strand sequence comprising or consisting of the nucleic acid sequence set forth in any one of SEQ ID NOS:1176-1288, 40, 108, and 166. In some embodiments, the isolated nucleic acid comprises an expression construct encoding a miRNA, such as a pri-miRNA, a pre-mRNA, an artificial miRNA, or a mature miRNA, wherein the miRNA comprises a guide strand sequence comprising or consisting of the nucleic acid sequence set forth in any one of SEQ ID NOS:1176-1288, 40, 108, and 166, with at least 1, 2, 3, 4, or 5 mismatches to the target ATXN2 mRNA sequence. In some embodiments, the miRNA is a pri-miRNA, a pre-mRNA, an artificial miRNA, or a mature miRNA wherein the miRNA comprises a guide strand sequence comprising or consisting of a nucleic acid sequence that is at least 60%, 70%, 75%, 80%, 85%, 90%, 95%, 97%, 99%, or 100% identical to any one of SEQ ID NOS:1176-1288, 40, 108, and 166. In some embodiments, the isolated nucleic acid comprises an expression construct encoding a miRNA, such as a pri-miRNA, a pre-mRNA, an artificial miRNA, or a mature miRNA, wherein the miRNA comprises a guide strand sequence comprising or consisting of at least 15, 16, 17, 18, 19, 20, 21, or 22 contiguous nucleotides of a sequence of any one of SEQ ID NOS:1176-1288, 40, 108, and 166, preferably wherein the guide strand sequence retains positions 2-7 (“seed sequence”) of the selected SEQ ID NO. In some embodiments, the isolated nucleic acid comprises an expression construct encoding a miRNA, such as a pri-miRNA, a pre-mRNA, an artificial miRNA, or a mature miRNA, wherein the miRNA comprises a guide strand sequence comprising or consisting of a sequence of any one of SEQ ID NOS:1176-1288, 40, 108, and 166, wherein 1, 2, 3, or 4 nucleotides at positions 19-22 differ from the selected SEQ ID NO (variant nucleotide(s)), such that the guide strand sequence is no longer complementary to the ATXN2 target sequence at the variant nucleotide(s).

In some embodiments, the isolated nucleic acid comprises an expression construct encoding a miRNA, such as a pri-miRNA, a pre-mRNA, an artificial miRNA, or a mature miRNA comprising a guide strand sequence of Table 23. In some embodiments, the isolated nucleic acid comprises an expression construct encoding a miRNA, such as a pri-miRNA, a pre-mRNA, an artificial miRNA, or a mature miRNA, comprising a guide strand sequence comprising or consisting of the nucleic acid sequence set forth in any one of SEQ ID NOS:1908-2007. In some embodiments, the isolated nucleic acid comprises an expression construct encoding a miRNA, such as a pri-miRNA, a pre-mRNA, an artificial miRNA, or a mature miRNA, wherein the miRNA comprises a guide strand sequence comprising or consisting of the nucleic acid sequence set forth in any one of SEQ ID NOS:1908-2007, with at least 1, 2, 3, 4, or 5 mismatches to the target ATXN2 mRNA sequence. In some embodiments, the miRNA is a pri-miRNA, a pre-mRNA, an artificial miRNA, or a mature miRNA wherein the miRNA comprises a guide strand sequence comprising or consisting of a nucleic acid sequence that is at least 60%, 70%, 75%, 80%, 85%, 90%, 95%, 97%, 99%, or 100% identical to any one of SEQ ID NOS:1908-2007. In some embodiments, the isolated nucleic acid comprises an expression construct encoding a miRNA, such as a pri-miRNA, a pre-mRNA, an artificial miRNA, or a mature miRNA, wherein the miRNA comprises a guide strand sequence comprising or consisting of at least 15, 16, 17, 18, 19, 20, 21, or 22 contiguous nucleotides of a sequence of any one of SEQ ID NOS: 1908-2007, preferably wherein the guide strand sequence retains positions 2-7 (“seed sequence”) of the selected SEQ ID NO. In some embodiments, the isolated nucleic acid comprises an expression construct encoding a miRNA, such as a pri-miRNA, a pre-mRNA, an artificial miRNA, or a mature miRNA, wherein the miRNA comprises a guide strand sequence comprising or consisting of a sequence of any one of SEQ ID NOS:1908-2007, wherein 1, 2, 3, or 4 nucleotides at positions 19-22 differ from the selected SEQ ID NO (variant nucleotide(s)), such that the guide strand sequence is no longer complementary to the ATXN2 target sequence at the variant nucleotide(s).

In some embodiments, the isolated nucleic acid comprises an expression construct encoding a miRNA, such as a pri-miRNA, a pre-mRNA, an artificial miRNA, or a mature miRNA comprising a guide strand sequence of Table 24. In some embodiments, the isolated nucleic acid comprises an expression construct encoding a miRNA, such as a pri-miRNA, a pre-mRNA, an artificial miRNA, or a mature miRNA, comprising a guide strand sequence comprising or consisting of the nucleic acid sequence set forth in any one of SEQ ID NOS:100, 112, 166, 202, 246, 306, 308, 314, 1180, 1185, 1196, 1200, 1211, 1213, 1215, 1216, 1224, 1811-1822, 1824-1827, 2015, 2065, 2083, 2152, 2203, and 2209. In some embodiments, the isolated nucleic acid comprises an expression construct encoding a miRNA, such as a pri-miRNA, a pre-mRNA, an artificial miRNA, or a mature miRNA, wherein the miRNA comprises a guide strand sequence comprising or consisting of the nucleic acid sequence set forth in any one of SEQ ID NOS:100, 112, 166, 202, 246, 306, 308, 314, 1180, 1185, 1196, 1200, 1211, 1213, 1215, 1216, 1224, 1811-1822, 1824-1827, 2015, 2065, 2083, 2152, 2203, and 2209, with at least 1, 2, 3, 4, or 5 mismatches to the target ATXN2 mRNA sequence. In some embodiments, the miRNA is a pri-miRNA, a pre-mRNA, an artificial miRNA, or a mature miRNA wherein the miRNA comprises a guide strand sequence comprising or consisting of a nucleic acid sequence that is at least 60%, 70%, 75%, 80%, 85%, 90%, 95%, 97%, 99%, or 100% identical to any one of SEQ ID NOS:100, 112, 166, 202, 246, 306, 308, 314, 1180, 1185, 1196, 1200, 1211, 1213, 1215, 1216, 1224, 1811-1822, 1824-1827, 2015, 2065, 2083, 2152, 2203, and 2209. In some embodiments, the isolated nucleic acid comprises an expression construct encoding a miRNA, such as a pri-miRNA, a pre-mRNA, an artificial miRNA, or a mature miRNA, wherein the miRNA comprises a guide strand sequence comprising or consisting of at least 15, 16, 17, 18, 19, 20, 21, or 22 contiguous nucleotides of a sequence of any one of SEQ ID NOS:100, 112, 166, 202, 246, 306, 308, 314, 1180, 1185, 1196, 1200, 1211, 1213, 1215, 1216, 1224, 1811-1822, 1824-1827, 2015, 2065, 2083, 2152, 2203, and 2209, preferably wherein the guide strand sequence retains positions 2-7 (“seed sequence”) of the selected SEQ ID NO. In some embodiments, the isolated nucleic acid comprises an expression construct encoding a miRNA, such as a pri-miRNA, a pre-mRNA, an artificial miRNA, or a mature miRNA, wherein the miRNA comprises a guide strand sequence comprising or consisting of a sequence of any one of SEQ ID NOS:100, 112, 166, 202, 246, 306, 308, 314, 1180, 1185, 1196, 1200, 1211, 1213, 1215, 1216, 1224, 1811-1822, 1824-1827, 2015, 2065, 2083, 2152, 2203, and 2209, wherein 1, 2, 3, or 4 nucleotides at positions 19-22 differ from the selected SEQ ID NO (variant nucleotide(s)), such that the guide strand sequence is no longer complementary to the ATXN2 target sequence at the variant nucleotide(s).

In some embodiments, the isolated nucleic acid comprises an expression construct encoding a miRNA, such as a pri-miRNA, a pre-mRNA, an artificial miRNA, or a mature miRNA comprising a guide strand sequence of Table 25. In some embodiments, the isolated nucleic acid comprises an expression construct encoding a miRNA, such as a pri-miRNA, a pre-mRNA, an artificial miRNA, or a mature miRNA, comprising a guide strand sequence comprising or consisting of the nucleic acid sequence set forth in any one of SEQ ID NOS:1185, 1816, 1213, 1819, 2083, 1215, 1216, 1811, and 314. In some embodiments, the isolated nucleic acid comprises an expression construct encoding a miRNA, such as a pri-miRNA, a pre-mRNA, an artificial miRNA, or a mature miRNA, wherein the miRNA comprises a guide strand sequence comprising or consisting of the nucleic acid sequence set forth in any one of SEQ ID NOS:1185, 1816, 1213, 1819, 2083, 1215, 1216, 1811, and 314, with at least 1, 2, 3, 4, or 5 mismatches to the target ATXN2 mRNA sequence. In some embodiments, the miRNA is a pri-miRNA, a pre-mRNA, an artificial miRNA, or a mature miRNA wherein the miRNA comprises a guide strand sequence comprising or consisting of a nucleic acid sequence that is at least 60%, 70%, 75%, 80%, 85%, 90%, 95%, 97%, 99%, or 100% identical to any one of SEQ ID NOS:1185, 1816, 1213, 1819, 2083, 1215, 1216, 1811, and 314. In some embodiments, the isolated nucleic acid comprises an expression construct encoding a miRNA, such as a pri-miRNA, a pre-mRNA, an artificial miRNA, or a mature miRNA, wherein the miRNA comprises a guide strand sequence comprising or consisting of at least 15, 16, 17, 18, 19, 20, 21, or 22 contiguous nucleotides of a sequence of any one of SEQ ID NOS:1185, 1816, 1213, 1819, 2083, 1215, 1216, 1811, and 314, preferably wherein the guide strand sequence retains positions 2-7 (“seed sequence”) of the selected SEQ ID NO. In some embodiments, the isolated nucleic acid comprises an expression construct encoding a miRNA, such as a pri-miRNA, a pre-mRNA, an artificial miRNA, or a mature miRNA, wherein the miRNA comprises a guide strand sequence comprising or consisting of a sequence of any one of SEQ ID NOS:1185, 1816, 1213, 1819, 2083, 1215, 1216, 1811, and 314, wherein 1, 2, 3, or 4 nucleotides at positions 19-22 differ from the selected SEQ ID NO (variant nucleotide(s)), such that the guide strand sequence is no longer complementary to the ATXN2 target sequence at the variant nucleotide(s).

In some embodiments, the isolated nucleic acid comprises an expression construct encoding a miRNA, such as a pri-miRNA, a pre-mRNA, an artificial miRNA, or a mature miRNA, comprising a guide strand sequence comprising or consisting of the nucleic acid sequence set forth in any one of SEQ ID NOS:1185, 1816, 1213, and 1811. In some embodiments, the isolated nucleic acid comprises an expression construct encoding a miRNA, such as a pri-miRNA, a pre-mRNA, an artificial miRNA, or a mature miRNA, wherein the miRNA comprises a guide strand sequence comprising or consisting of the nucleic acid sequence set forth in any one of SEQ ID NOS:1185, 1816, 1213, and 1811, with at least 1, 2, 3, 4, or 5 mismatches to the target ATXN2 mRNA sequence. In some embodiments, the miRNA is a pri-miRNA, a pre-mRNA, an artificial miRNA, or a mature miRNA wherein the miRNA comprises a guide strand sequence comprising or consisting of a nucleic acid sequence that is at least 60%, 70%, 75%, 80%, 85%, 90%, 95%, 97%, 99%, or 100% identical to any one of SEQ ID NOS: 1185, 1816, 1213, and 1811. In some embodiments, the isolated nucleic acid comprises an expression construct encoding a miRNA, such as a pri-miRNA, a pre-mRNA, an artificial miRNA, or a mature miRNA, wherein the miRNA comprises a guide strand sequence comprising or consisting of at least 15, 16, 17, 18, 19, 20, 21, or 22 contiguous nucleotides of a sequence of any one of SEQ ID NOS:1185, 1816, 1213, and 1811, preferably wherein the guide strand sequence retains positions 2-7 (“seed sequence”) of the selected SEQ ID NO. In some embodiments, the isolated nucleic acid comprises an expression construct encoding a miRNA, such as a pri-miRNA, a pre-mRNA, an artificial miRNA, or a mature miRNA, wherein the miRNA comprises a guide strand sequence comprising or consisting of a sequence of any one of SEQ ID NOS:1185, 1816, 1213, and 1811, wherein 1, 2, 3, or 4 nucleotides at positions 19-22 differ from the selected SEQ ID NO (variant nucleotide(s)), such that the guide strand sequence is no longer complementary to the ATXN2 target sequence at the variant nucleotide(s).

In some embodiments, the isolated nucleic acid comprises an expression construct encoding an artificial miRNA comprising a guide strand sequence according to any of the embodiments described herein, contained within a miR backbone sequence. In some embodiments, the guide strand sequence and passenger strand sequence of the artificial miRNA are contained with a miRNA backbone sequence. In some embodiments, the miRNA backbone sequence is contained within a miR-155 backbone sequence, a miR-155E backbone sequence, a miR-155M backbone sequence, a miR1-1 backbone sequence, a miR-1-1_M backbone sequence, a miR-100 backbone sequence, a miR-100_M backbone sequence, a miR-190A backbone sequence, a miR-124 backbone sequence, a miR-124_M backbone sequence, a miR-16-2 backbone sequence, a miR-132 backbone sequence, a miR-9 backbone sequence, a miR-138-2 backbone sequence, a miR-122 backbone sequence, a miR-122_M backbone sequence, a miR-130a backbone sequence, a miR-128 backbone sequence, a miR-144 backbone sequence, a miR-451a backbone sequence, or a miR-223 backbone sequence. In some embodiments, the terminal loop, stem, 5′ flanking segment, 3′ flanking segment, or any combination thereof of the miR-155 backbone sequence, miR1-1 backbone sequence, miR-100 backbone sequence, miR-190A backbone sequence, miR-124 backbone sequence, miR-16-2 backbone sequence, miR-132 backbone sequence, miR-9 backbone sequence, miR-138-2 backbone sequence, miR-122 backbone sequence, miR-130a backbone sequence, miR-128 backbone sequence, miR-144 backbone sequence, miR-451a backbone sequence, or miR-223 backbone sequence is modified (e.g., nucleotide insertion, deletion, substitution, mismatch, wobble, or any combination thereof).

In some embodiments, the isolated nucleic acid comprises an expression construct encoding a miRNA (pri-miRNA, a pre-mRNA, an artificial miRNA, or a mature miRNA) comprising a guide strand sequence and corresponding passenger strand sequence comprising or consisting of any one of the duplex sequences set forth in Tables 1, 19, 23, and 24. In some embodiments, the passenger strand sequence of the miRNA comprises a sequence that is 100% complementary or perfectly complementary to the guide strand sequence. For example, the encoded guide strand sequence may comprise of consist of a sequence of SEQ ID NO: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, or 436 (guide sequences in Table 1), and the encoded passenger strand sequence may comprise or consist of a sequence of SEQ ID NO: 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, or 435, respectively (passenger sequences in Table 1). In some embodiments, the passenger strand sequence of the miRNA is not 100% complementary or to the guide strand sequence. For example, a guide strand sequence may comprise or consist of a sequence of SEQ ID NO: 1176 and the corresponding passenger strand sequence may comprise or consist of a sequence of SEQ ID NO:1289 (see, Table 19).

In some embodiments, the isolated nucleic acid comprises an expression construct encoding a miRNA (pri-miRNA, a pre-mRNA, an artificial miRNA, or a mature miRNA) comprising a guide strand sequence comprising or consisting of any one of SEQ ID NOS: 12, 14, 40, 60, 100, 104, 108, 112, 124, 126, 128, 166, 198, 220, 242, 302, 306, 308, 330, 336, and 362, and a passenger strand sequence of comprising a sequence that is 100% complementary or perfectly complementary to the guide strand sequence. For example, the encoded guide strand sequence may comprise or consist of a sequence of SEQ ID NO: 12, 14, 40, 60, 100, 104, 108, 112, 124, 126, 128, 166, 198, 220, 242, 302, 306, 308, 330, 336, or 362, and the encoded passenger strand sequence may comprise or consist of a sequence of SEQ ID NO: 11, 13, 39, 59, 99, 103, 107, 111, 123, 125, 127, 165, 197, 219, 241, 301, 305, 307, 329, 335, or 361, respectively.

In some embodiments, the isolated nucleic acid comprises an expression construct encoding a miRNA (pri-miRNA, a pre-mRNA, an artificial miRNA, or a mature miRNA) wherein the miRNA comprises a guide strand sequence comprising or consisting of any one of SEQ ID NOS: 14, 40, 100, 108, 112, 128, 166, 198, 242, 308, 336, and 362, and a passenger strand sequence comprising a sequence that is 100% complementary or perfectly complementary to the guide strand. For example, the encoded guide strand sequence may comprise or consist of a sequence of SEQ ID NO: 14, 40, 100, 108, 112, 128, 166, 198, 242, 308, 336, or 362, and the encoded passenger strand sequence may comprise or consisting of a sequence of SEQ ID NO: 13, 39, 99, 107, 111, 127, 165, 197, 241, 307, 335, or 361, respectively.

In some embodiments, the isolated nucleic acid comprises an expression construct encoding a miRNA (pri-miRNA, a pre-mRNA, an artificial miRNA, or a mature miRNA), wherein the miRNA comprises a guide strand sequence comprising or consisting of any one of the guide sequences of Tables 1, 19, 23, and 24, and the passenger strand sequence comprises or consists of a corresponding passenger sequence of Tables 1, 19, 23, and 24 that has 1, 2, 3, 4, 5, 6, 7, 8, 9, 10, or more insertions, deletions, substitutions, mismatches, wobbles, or any combination thereof relative to the passenger strand sequence of Tables 1, 19, 23, and 24. In some embodiments, the isolated nucleic acid comprises an expression construct encoding a miRNA (pri-miRNA, a pre-mRNA, an artificial miRNA, or a mature miRNA), wherein the miRNA comprises a guide strand sequence comprising or consisting of any one of SEQ ID NOs: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, and a passenger strand sequence comprising or consisting of a sequence that has 1, 2, 3, 4, 5, 6, 7, 8, 9, 10, or more insertions, deletions, substitutions, mismatches, wobbles, or any combination thereof relative to the corresponding passenger strand sequence of SEQ ID NOS: 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435 respectively. In some embodiments, a mismatch is a G→C, C→G, A→T, or T→A conversion in the encoded passenger strand sequence. In some embodiments, a mismatch (to create a bulge with the guide strand) is a G→T, C→A, A→C, or T→G conversion in the encoded passenger strand sequence. In some embodiments, a wobble is a G-U wobble, wherein a C is converted to a T in the encoded passenger strand sequence. In some embodiments, the passenger strand sequence is modified according to the rules of Table 8.

In some embodiments, the isolated nucleic acid comprises an expression construct encoding a miRNA (pri-miRNA, a pre-mRNA, an artificial miRNA, or a mature miRNA) wherein the miRNA comprises a guide strand sequence comprising or consisting of any one of SEQ ID NOS: 12, 14, 40, 60, 100, 104, 108, 112, 124, 126, 128, 166, 198, 220, 242, 302, 306, 308, 330, 336, and 362, and a passenger strand sequence comprising or consisting of a sequence that has 1, 2, 3, 4, 5, 6, 7, 8, 9, 10, or more insertions, deletions, substitutions, mismatches, wobbles, or any combination thereof, relative to the passenger strand sequence comprising or consisting of SEQ ID NOS: 11, 13, 39, 59, 99, 103, 107, 11, 123, 125, 127, 165, 197, 219, 241, 301, 305, 307, 329, 335, and 361, respectively. In some embodiments, a mismatch is a G→C, C→G, A→T, or T→A conversion in the passenger strand sequence. In some embodiments, a mismatch (to create a bulge with the guide strand) is a G→T, C→A, A→C, or T→G conversion in the passenger strand sequence. In some embodiments, a wobble is a G-U wobble, wherein a C is converted to a T in the passenger strand sequence. In some embodiments, the passenger strand sequence is modified according to the rules of Table 8.

In some embodiments, the isolated nucleic acid comprises an expression construct encoding a miRNA (pri-miRNA, a pre-mRNA, an artificial miRNA, or a mature miRNA) wherein the miRNA comprises a guide strand sequence comprising or consisting of any one of SEQ ID NOS: 14, 40, 100, 108, 112, 128, 166, 198, 242, 308, 336, and 362, and a passenger strand sequence comprising a sequence that has 1, 2, 3, 4, 5, 6, 7, 8, 9, 10, or more insertions, deletions, substitutions, mismatches, wobbles, or any combination thereof relative to the passenger strand sequence comprising or consisting of SEQ ID NOS: 13, 39, 99, 107, 111, 127, 165, 197, 241, 307, 335, and 361, respectively. In some embodiments, a mismatch is a G→C, C→G, A→T, or T→A conversion in the encoded passenger strand sequence. In some embodiments, a mismatch (to create a bulge with the guide strand) is a G→T, C→A, A→C, or T→G conversion in the encoded passenger strand sequence. In some embodiments, a wobble is a G-U wobble, wherein a C is converted to a T in the encoded passenger strand sequence. In some embodiments, the passenger strand sequence is modified according to the rules of Table 8.

In some embodiments, the isolated nucleic acid comprises an expression construct encoding an artificial miRNA comprising a guide strand sequence according to any of the embodiments described herein, contained within a miR-155M backbone sequence, miR-155E backbone sequence, miR1-1 backbone sequence, miR-100 backbone sequence, miR-124 backbone sequence, mIR-138-2 backbone sequence, miR-122 backbone sequence, miR-128 backbone sequence, miR-130a backbone sequence, or miR-16-2 backbone sequence, wherein the artificial miRNA comprises a passenger strand sequence that is modified according to Table 8. In some embodiments, the passenger strand sequence comprises a mismatch, wherein a mismatch is a G→C, C→G, A→T, or T→A conversion in the passenger strand sequence; a mismatch (to create a bulge with the guide strand) is a G→T, C→A, A→C, or T→G conversion in the passenger strand sequence; and a wobble is a G-U wobble, wherein a C is converted to a T in the passenger strand sequence.

In some embodiments, the isolated nucleic acid comprises an expression construct encoding an artificial miRNA comprising or consisting of a nucleic acid sequence set forth in any one of Tables 3, 9, 11, 1923, 24, and 25. In some embodiments, the isolated nucleic acid comprises an expression construct encoding an artificial miRNA comprising or consisting of any one of SEQ ID NOS: 443-490, 1109-1111, 1114, 1121-1168, 1405-1520, 1908-2007, 2011, 2017, 2021, 2025, 2027, 2031, 2035, 2039, 2041, 2045, 2049, 2053, 2057, 2061, 2067, 2071, 2075, 2079, 2085, 2089, 2093, 2097, 2101, 2105, 2109, 2113, 2117, 2120, 2124, 2128, 2132, 2136, 2140, 2144, 2148, 2154, 2158, 2162, 2166, 2170, 2174, 2176, 2180, 2182, 2184, 2187, 2189, 2191, 2193, 2195, 2197, 2199, 2205, 2211, 2261, 2263, 2265, and 2267.

In some embodiments, the isolated nucleic acid comprises an expression construct encoding an artificial miRNA that comprises or consists of a nucleic acid sequence set forth in Table 3. In some embodiments, the isolated nucleic acid comprises an expression construct encoding an artificial miRNA that comprises or consists of a nucleic acid sequence of any one of SEQ ID NOS:443-490.

In some embodiments, the isolated nucleic acid comprises an expression construct encoding an artificial miRNA that comprises or consists of a nucleic acid sequence set forth in Table 9. In some embodiments, the isolated nucleic acid comprises an expression construct encoding an artificial miRNA that comprises or consists of a nucleic acid sequence of any one of SEQ ID NOS:1109-1111, and 1114.

In some embodiments, the isolated nucleic acid comprises an expression construct encoding an artificial miRNA that comprises or consists of a nucleic acid sequence set forth in Table 11. In some embodiments, the isolated nucleic acid comprises an expression construct encoding an artificial miRNA that comprises or consists of a nucleic acid sequence of any one of SEQ ID NOS:1121-1168.

In some embodiments, the isolated nucleic acid comprises an expression construct encoding an artificial miRNA that comprises or consists of a nucleic acid sequence set forth in Table 19. In some embodiments, the isolated nucleic acid comprises an expression construct encoding an artificial miRNA that comprises or consists of a nucleic acid sequence of any one of SEQ ID NOS:1405-1520.

In some embodiments, the isolated nucleic acid comprises an expression construct encoding an artificial miRNA that comprises or consists of a nucleic acid sequence set forth in Table 23. In some embodiments, the isolated nucleic acid comprises an expression construct encoding an artificial miRNA that comprises or consists of a nucleic acid sequence of any one of SEQ ID NOS:1908-2007.

In some embodiments, the isolated nucleic acid comprises an expression construct encoding an artificial miRNA that comprises or consists of a nucleic acid sequence set forth in Table 24. In some embodiments, the isolated nucleic acid comprises an expression construct encoding an artificial miRNA that comprises or consists of a nucleic acid sequence of any one of SEQ ID NOS:1908-1934, 1936-1977, 1979-1982, 1984-1994, 1997, 1998, 2000, 2001, 2005-2007, 2011, 2017, 2021, 2025, 2027, 2031, 2035, 2039, 2041, 2045, 2049, 2053, 2057, 2061, 2067, 2071, 2075, 2079, 2085, 2089, 2093, 2097, 2101, 2105, 2109, 2113, 2117, 2120, 2124, 2128, 2132, 2136, 2140, 2144, 2148, 2154, 2158, 2162, 2166, 2170, 2174, 2176, 2180, 2182, 2184, 2187, 2189, 2191, 2193, 2195, 2197, 2199, 2205, 2211, 2261, 2263, 2265, and 2267.

In some embodiments, the isolated nucleic acid comprises an expression construct encoding an artificial miRNA that comprises or consists of a nucleic acid sequence set forth in Table 25. In some embodiments, the isolated nucleic acid comprises an expression construct encoding an artificial miRNA that comprises or consists of a nucleic acid sequence of any one of SEQ ID NOS:1915, 1982, 1965, 1937, 1985, 1921, and 2021.

In some embodiments, expression constructs encoding the inhibitory nucleic acids that target ATXN2 mRNA comprises or consists of any of the guide strand sequences or artificial miRNA sequences disclosed in DNA format. For example, Tables 9, 11, 23, and 24 provide amiRNA sequences in DNA format, which DNA sequence may be inserted into expression constructs. Alternatively, amiRNA sequences provided herein can be converted to DNA format by replacing each “U” nucleotide with a “T” nucleotide.

In some embodiments, the expression construct encodes two or more inhibitory nucleic acids that target an ATXN2 mRNA transcript described herein. In some embodiments, the expression construct encodes an inhibitory nucleic acid that targets ATXN2 transcript and an inhibitory nucleic acid that targets a second target transcript other than ATXN2. In some embodiments, the second target transcript is C9ORF72. Examples of inhibitory nucleic acids targeting C9ORF72 are described in US Patent Publication US2019/0316126 (incorporated by reference in its entirety). In some embodiments, the expression construct encodes an inhibitory nucleic acid that targets ATXN2 transcript and encodes a therapeutic polypeptide or protein.

In some embodiments, the expression construct is monocistronic. In some embodiments, the expression construct is polycistronic (e.g., expression construct encodes two or more peptides or polypeptides). In some embodiments, a nucleic acid sequence encoding a first gene product (e.g., inhibitory nucleic acid targeting ATXN2 mRNA) and a nucleic acid sequence encoding a second gene product within an expression construct are separated by an internal ribosome entry site (IRES), furin cleavage site, or viral 2A peptide. In some embodiments, a viral 2A peptide is a porcine teschovirus-1 (P2A), Thosea asigna virus (T2A), equine rhinitis A virus (E2A), foot-and-mouth disease virus (F2A), B. mori cytoplasmic polyhedrosis virus (BmCPV 2A), B. mori flacherie virus (BmIFV 2A), or variant thereof.

In some embodiments, the expression construct further comprises one or more expression control sequences (regulatory sequences) operably linked with the transgene (e.g., nucleic acid encoding an artificial miRNA). “Operably linked” sequences include expression control sequences that are contiguous with the transgene or act in trans or at a distance from the transgene to control its expression. Examples of expression control sequences include transcription initiation sequences, termination sequences, promoter sequences, enhancer sequences, repressor sequences, splice site sequences, polyadenylation (polyA) signal sequences, or any combination thereof.

In some embodiments, a promoter is an endogenous promoter, synthetic promoter, constitutive promoter, inducible promoter, tissue-specific promoter (e.g., CNS-specific), or cell-specific promoter (neurons, glial cells, or astrocytes). Examples of constitutive promoters include, Rous sarcoma virus (RSV) LTR promoter (optionally with the RSV enhancer), cytomegalovirus (CMV) promoter (optionally with the CMV enhancer), SV40 promoter, and dihydrofolate reductase promoter. Examples of inducible promoters include zinc-inducible sheep metallothionine (MT) promoter, dexamethasone (Dex)-inducible mouse mammary tumor virus (MMTV) promoter, T7 polymerase promoter system, the ecdysone insect promoter, tetracycline-repressible system, tetracycline-inducible system, RU486-inducible system, and the rapamycin-inducible system. Further examples of promoters that may be used include, for example, chicken beta-actin promoter (CBA promoter), a CAG promoter, a H1 promoter, a CD68 promoter, a JeT promoter, synapsin promoter, RNA pol II promoter, or a RNA pol III promoter (e.g., U6, H1, etc.). In some embodiments, the promoter is a tissue-specific RNA pol II promoter. In some embodiments, the tissue-specific RNA pol II promoter is derived from a gene that exhibits neuron-specific expression. In some embodiments, the neuron-specific promoter is a synapsin 1 promoter or synapsin 2 promoter.

In some embodiments, the promoter is an H1 promoter comprising or consisting of the sequence set forth in nucleotides 113-203 of SEQ ID NO:1522. In some embodiments, the promoter is an H1 promoter comprising or consisting of the sequence set forth in nucleotides 1798-1888 of SEQ ID NO:1521. In some embodiments, the promoter is an H1 promoter comprising or consisting of the sequence set forth in nucleotides 113-343 of any one of SEQ ID NOS:2257-2260. In some embodiments, the promoter is an H1 promoter comprising or consisting of the sequence set forth in nucleotides 244-343 of any one of SEQ ID NOS:2257-2260.

In some embodiments, the sequence encoding the inhibitory nucleic acid of the present disclosure is positioned in an untranslated region of an expression construct. In some embodiments, the sequence encoding the inhibitory nucleic acid of the present disclosure is positioned in an intron, a 5′ untranslated region (5′UTR), or a 3′ untranslated region (3′UTR) of the expression construct. In some embodiments, the sequence encoding the inhibitory nucleic acid of the present disclosure is positioned in an intron downstream of the promoter and upstream of an expressed gene.

In some embodiments, the isolated nucleic acid comprises an expression construct encoding an inhibitory nucleic, flanked by two AAV inverted terminal repeats (ITRs) (e.g., 5′ ITR and 3′ ITR). In some embodiments, each AAV ITR is a full length ITR (e.g., approximately 145 bp in length, and containing functional Rep binding site (RBS) and terminal resolution site (trs)). In some embodiments, one of the ITRs is truncated (e.g., shortened or not full-length). In some embodiments, a truncated ITR lacks a functional terminal resolution site (trs) and is used for production of self-complementary AAV vectors (scAAV vectors). In some embodiments, a truncated ITR is a truncated version of AAV2 ITR referred to as AITR (D-sequence and TRS are deleted). In some embodiments, the ITRs are selected from AAV serotypes of AAV1, AAV2, AAV3, AAV4, AAV5, AAV6, AAV7, AAV8, AAV9, AAV9.47, AAV9(hu14), AAV10, AAV11, AAV 12, AAVrh8, AAVrh10, AAV-DJ8, AAV-DJ, AAV-PUP.A, AAV-PHP.B, AAVPHP.B2, AAVPHP.B3, AAVPHP.N/PHP.B-DGT, AAVPHP.B-EST, AAVPHP.B-GGT, AAVPHP.B-ATP, AAVPHP.B-ATT-T, AAVPHP.B-DGT-T, AAVPHP.B-GGT-T, AAVPHP.B-SGS, AAVPHP.B-AQP, AAVPHP.B-QQP, AAVPHP.B-SNP(3), AAVPHP.B-SNP, AAVPHP.B-QGT, AAVPHP.B-NQT, AAVPHP.B-EGS, AAVPHP.B-SGN, AAVPHP.B-EGT, AAVPHP.B-DST, AAVPHP.B-DST, AAVPHP.B-STP, AAVPHP.B-PQP, AAVPHP.B-SQP, AAVPHP.B-QLP, AAVPHP.B-TMP, AAVPHP.B-TTP, AAVPHP.S/G2A12, AAVG2A 15/G2A3, AAVG2B4, AAVG2B5, and variants thereof.

In some embodiments, the isolated nucleic acid molecule comprising an expression construct encoding an inhibitory nucleic acid that inhibits expression or activity of ATXN2 comprises the nucleotide sequence set forth in any one of SEQ ID NOS:2257-2260. In some embodiments, the isolated nucleic acid molecule comprising an expression construct encoding an inhibitory nucleic acid that inhibits expression or activity of ATXN2 comprises the nucleotide sequence set forth in SEQ ID NO:2257. In some embodiments, the isolated nucleic acid molecule comprising an expression construct encoding an inhibitory nucleic acid that inhibits expression or activity of ATXN2 comprises the nucleotide sequence set forth in SEQ ID NO:2258. In some embodiments, the isolated nucleic acid molecule comprising an expression construct encoding an inhibitory nucleic acid that inhibits expression or activity of ATXN2 comprises the nucleotide sequence set forth in SEQ ID NO:2259. In some embodiments, the isolated nucleic acid molecule comprising an expression construct encoding an inhibitory nucleic acid that inhibits expression or activity of ATXN2 comprises the nucleotide sequence set forth in SEQ ID NO:2260.

Additional isolated nucleic acid molecules comprising an expression construct encoding an inhibitory nucleic acid that inhibits expression or activity of ATXN2 may be constructed using the nucleotide sequence set forth in any one of SEQ ID NOS:2257-2260, by substituting the desired inhibitory nucleic acid sequence (e.g., artificial miRNA cassette) of the present disclosure into nucleotide positions 344-481 of any one of SEQ ID NOS:2257-2260.

### Vectors and Host Cells

Inhibitory nucleic acid molecules (siRNAs, shRNAs, miRNAs) described herein can be encoded by vectors. The use of vectors, e.g., AAV, for expressing inhibitory nucleic acids of the present disclosure may allow for continual or controlled expression of inhibitory nucleic acid in the subject, rather than multiple doses of isolated inhibitory nucleic acids to the subject. The present disclosure provides a vector comprising an isolated nucleic acid comprising an expression construct encoding an inhibitory nucleic described herein. A vector can be a plasmid, cosmid, phagemid, bacterial artificial chromosome (BAC) or viral vector. Examples of viral vectors include herpesvirus (HSV) vectors, retroviral vectors, adenoviral vectors, adeno-associated viral (AAV) vectors, lentiviral vectors, baculoviral vectors, and the like. In some embodiments, a retroviral vector is a mouse stem cell virus, murine leukemia virus (e.g. Moloney murine leukemia virus vector), feline leukemia virus, feline sarcoma virus, or avian reticuloendotheliosis virus vector. In some embodiments, a lentiviral vector is a HIV (human immunodeficiency virus, including HIV type 1 and HIV type 2, equine infectious anemia virus, feline immunodeficiency virus (FIV), bovine immune deficiency virus (BIV), and simian immunodeficiency virus (SIV), equine infectious anemia virus, or Maedi-Visna viral vector.

In some embodiments, the vector is an AAV (AAV) vector, such as a recombinant AAV (rAAV) vector, which is produced by recombinant methods. AAV is a single-stranded, non-enveloped DNA virus having a genome that encodes proteins for replication (rep) and the capsid (Cap), flanked by two ITRs, which serve as the origin of replication of the viral genome. AAV also contains a packaging sequence, allowing packaging of the viral genome into an AAV capsid. A recombinant AAV vector (rAAV) may be obtained from the wild type genome of AAV by using molecular methods to remove the all or part of the wild type genome (e.g., Rep, Cap) from the AAV, and replacing with a non-native nucleic acid, such as a heterologous nucleic acid sequence (e.g., a nucleic acid molecule encoding an inhibitory nucleic acid). Typically, for AAV one or both inverted terminal repeat (ITR) sequences are retained in the AAV vector. In some embodiments, the rAAV vector comprises an expression construct encoding an inhibitory nucleic acid of the present disclosure flanked by two cis-acting AAV ITRs (5′ ITR and 3′ ITR). Functional ITR sequences are necessary for the rescue, replication and packaging of the AAV viral particle. Thus, an AAV vector is defined herein to include at least those sequences required in cis for replication and packaging (e.g., functional ITRs) of the virus. In some embodiments, each AAV ITR is a full length ITR (e.g., approximately 145 bp in length, and containing functional Rep binding site (RBS) and terminal resolution site (trs)). In some embodiments, one or both of the ITRs is is modified, e.g., by insertion, deletion, or substitution, provided that the ITRs provide for functional rescue, replication, and packaging. In some embodiments, a modified ITR lacks a functional terminal resolution site (trs) and is used for production of self-complementary AAV vectors (scAAV vectors). In some embodiments, a modified ITR is a truncated version of AAV2 ITR referred to as AITR (D-sequence and TRS are deleted).

In some embodiments, the AAV vector comprises a 5′ ITR comprising or consisting of nucleotides 1-106 of any one of SEQ ID NOS:2257-2260. In some embodiments, the AAV vector comprises a 3′ ITR comprising or consisting of nucleotides 2192-2358 of any one of SEQ ID NOS:2257-2260. In some embodiments, the AAV vector comprises: a 5′ ITR comprising or consisting of nucleotides 1-106 of SEQ ID NO:2257 and a 3′ ITR comprising or consisting of nucleotides 2192-2358 of SEQ ID NO:2257; a 5′ ITR comprising or consisting of nucleotides 1-106 of SEQ ID NO:2258 and a 3′ ITR comprising or consisting of nucleotides 2192-2358 of SEQ ID NO:2258; a 5′ ITR comprising or consisting of nucleotides 1-106 of SEQ ID NO:2259 and a 3′ ITR comprising or consisting of nucleotides 2192-2358 of SEQ ID NO:2259; or a 5′ ITR comprising or consisting of nucleotides 1-106 of SEQ ID NO:2260 and a 3′ ITR comprising or consisting of nucleotides 2192-2358 of SEQ ID NO:2260.

In some embodiments, the rAAV vector is a mammalian serotype AAV vector (e.g., AAV genome and ITRs derived from mammalian serotype AAV), including a primate serotype AAV vector or human serotype AAV vector. In some embodiments, the AAV vector is a chimeric AAV vector. In some embodiments, the ITRs are selected from AAV serotypes of AAV1, AAV2, AAV3, AAV4, AAV5, AAV6, AAV7, AAV8, AAV9, AAV9.47, AAV9(hu14), AAV10, AAV11, AAV 12, AAVrh8, AAVrh10, AAV-DJ8, AAV-DJ, AAV-PUPA, AAV-PHP.B, AAVPHP.B2, AAVPHP.B3, AAVPHP.N/PHP.B-DGT, AAVPHP.B-EST, AAVPHP.B-GGT, AAVPHP.B-ATP, AAVPHP.B-ATT-T, AAVPHP.B-DGT-T, AAVPHP.B-GGT-T, AAVPHP.B-SGS, AAVPHP.B-AQP, AAVPHP.B-QQP, AAVPHP.B-SNP(3), AAVPHP.B-SNP, AAVPHP.B-QGT, AAVPHP.B-NQT, AAVPHP.B-EGS, AAVPHP.B-SGN, AAVPHP.B-EGT, AAVPHP.B-DST, AAVPHP.B-DST, AAVPHP.B-STP, AAVPHP.B-PQP, AAVPHP.B-SQP, AAVPHP.B-QLP, AAVPHP.B-TMP, AAVPHP.B-TTP, AAVPHP.S/G2A12, AAVG2A 15/G2A3, AAVG2B4, AAVG2B5, and variants thereof.

Other expression control sequences may be present in the rAAV vector operably linked to the inhibitory nucleic acid, including one or more of transcription initiation sequences, termination sequences, promoter sequences, enhancer sequences, repressor sequences, splice site sequences, polyadenylation (polyA) signal sequences, or any combination thereof.

AAV preferentially packages a full-length genome, i.e., one that is approximately the same size as the native genome, and is not too big or too small. However, expression cassettes encoding inhibitory nucleic acid sequences are rather small. To avoid packaging of fragmented genomes, a stuffer sequence may be linked to an expression construct encoding inhitory nucleic acids of the present disclosure and flanked by the 5′ ITR and 3′ ITR to expand the packagable genome, resulted in a genome whose size was near-normal in length between the ITRs. In some embodiments, the rAAV vector comprising a stuffer sequence and expression cassette encoding an inhibitory nucleic acid sequence of the present disclosure has a total length of about 4.7 kb between the 5′ ITR and 3′ ITR. In some embodiments, the rAAV vector is a self-complementary rAAV vector comprising a stuffer sequence and expression cassette encoding an inhibitory nucleic acid sequence of the present disclosure and has a total length of about 2.4 kb between the 5′ ITR and 3′ ITR. An exemplary stuffer sequence for use in the rAAV vectors of the present disclosure includes a sequence comprising or consisting of nucleotides 348-2228 of SEQ ID NO:1522 and a sequence comprising or consisting of nucleotides 489-2185 of any one of SEQ ID NOS:2257-2260.

rAAV vectors may have one or more AAV wild type genes deleted in whole or in part. In some embodiments the rAAV vector is replication defective. In some embodiments, the rAAV vector lacks a functional Rep protein and/or capsid protein. In some embodiments, the rAAV vector is a self-complementary AAV (scAAV) vector.

In some embodiments, the rAAV vector comprises from 5′ ITR to 3′ ITR the nucleotide sequence set forth in any one of SEQ ID NOS:2257-2260. In some embodiments, the rAAV vector comprises from 5′ ITR to 3′ ITR the nucleotide sequence set forth in SEQ ID NO:2257. In some embodiments, the rAAV vector comprises from 5′ ITR to 3′ ITR the nucleotide sequence set forth in SEQ ID NO:2258. In some embodiments, the rAAV vector comprises from 5′ ITR to 3′ ITR the nucleotide sequence set forth in SEQ ID NO:2259. In some embodiments, the rAAV vector comprises the nucleotide sequence set forth in SEQ ID NO:2260.

Recombinant AAV vectors of the present disclosure may be encapsidated by one or more AAV capsid proteins to form a rAAV particle. A “rAAV particle” or “rAAV virion” refers to an infectious, replication-defective virus including an AAV protein shell, encapsidating a rAAV vector comprising a transgene of interest, which is flanked on each side by a 5′ AAV ITR and 3′ AAV ITR. A rAAV particle is produced in a suitable host cell which has had sequences specifying a rAAV vector, AAV helper functions and accessory functions introduced therein to render the host cell capable of encoding AAV polypeptides that are required for packaging the rAAV vector (containing the transgene sequence of interest) into infectious rAAV particles for subsequent gene delivery.

Methods of packaging recombinant AAV vector into AAV capsid proteins using host cell culture are known in the art. In some embodiments, one or more of the required components for packaging the rAAV vector, (e.g., Rep sequence, cap sequence, and/or accessory functions) may be provided by a stable host cell that has been engineered to to contain the one or more required components (e.g., by a vector). Expression of the required components for AAV packaging may be under control of an inducible or constitutive promoter in the host packaging cell. AAV helper vectors are commonly used to provide transient expression of AAV rep and/or cap genes, which function in trans, to complement missing AAV functions that are necessary for AAV replication. In some embodiments, AAV helper vectors lack AAV ITRs and can neither replicate nor package themselves. AAV helper vectors can be in the form of a plasmid, phage, transposon, cosmid, virus, or virion.

In some embodiments, rAAV particles may be produced using the triple transfection method (see, e.g., U.S. Pat. No. 6,001,650, incorporated herein by reference in its entirety). In this approach, the rAAV particles are produced by transfecting a host cell with a rAAV vector (comprising a transgene) to be packaged into rAAV particles, an AAV helper vector, and an accessory function vector. In some embodiments, the AAV helper function vector supports efficient AAV vector production without generating any detectable wild-type AAV virions (e.g., AAV virions containing functional rep and cap genes). The accessory function vector encodes nucleotide sequences for non-AAV derived viral and/or cellular functions upon which AAV is dependent for replication (e.g., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus. In some embodiments, a double transfection method, wherein the AAV helper function and accessory function are cloned on a single vector, which is used to generate rAAV particles.

The AAV capsid is an important element in determining these tissue-specificity of the rAAV particle. Thus, a rAAV particle having a capsid tissue specificity can be selected. In some embodiments, the rAAV particle comprises a capsid protein selected from a AAV serotype of AAV1, AAV2, AAV3, AAV4, AAV5, AAV6, AAV7, AAV8, AAV9, AAV9.47, AAV9(hu14), AAV10, AAV11, AAV 12, AAVrh8, AAVrh10, AAV-DJ8, AAV-DJ, AAV-PUPA, AAV-PHP.B, AAVPHP.B2, AAVPHP.B3, AAVPHP.N/PHP.B-DGT, AAVPHP.B-EST, AAVPHP.B-GGT, AAVPHP.B-ATP, AAVPHP.B-ATT-T, AAVPHP.B-DGT-T, AAVPHP.B-GGT-T, AAVPHP.B-SGS, AAVPHP.B-AQP, AAVPHP.B-QQP, AAVPHP.B-SNP(3), AAVPHP.B-SNP, AAVPHP.B-QGT, AAVPHP.B-NQT, AAVPHP.B-EGS, AAVPHP.B-SGN, AAVPHP.B-EGT, AAVPHP.B-DST, AAVPHP.B-DST, AAVPHP.B-STP, AAVPHP.B-PQP, AAVPHP.B-SQP, AAVPHP.B-QLP, AAVPHP.B-TMP, AAVPHP.B-TTP, AAVPHP.S/G2A12, AAVG2A 15/G2A3, AAVG2B4, AAVG2B5, and variants thereof. In some embodiments, the AAV capsid is selected from a serotype that is capable of crossing the blood-brain barrier, e.g., AAV9, AAVrh.10, AAV-PHP-B, or a variant thereof. In some embodiments, the AAV capsid is a chimeric AAV capsid. In some embodiments, the AAV particle is a pseudotyped AAV, having capsid and genome from different AAV serotypes.

In some embodiments, the rAAV particle is capable of transducing cells of the CNS. In some embodiments, the rAAV particle is capable of transducing non-neuronal cells or neuronal cells of the CNS. In some embodiments, the CNS cell is a neuron, glial cell, astrocyte, or microglial cell.

In another aspect, the present disclosure provides host cells transfected with the rAAV comprising the inhibitory nucleic acids or vectors described herein. In some embodiments, the host cell is a prokaryotic cell or a eukaryotic cell. In some embodiments, the host cell is a mammalian cell (e.g., HEK293T, COS cells, HeLa cells, KB cells), bacterial cell (E. coli), yeast cell, insect cell (Sf9, Sf21, Drosophila, mosquito), etc.

### Pharmaceutical Compositions

In some aspects, the disclosure provides pharmaceutical compositions comprising an inhibitory nucleic acid, isolated nucleic acid comprising an expression construct, or vector as described herein and a pharmaceutically acceptable carrier. As used herein, the term “pharmaceutically acceptable” refers to those compounds, materials, compositions, and/or dosage forms which are, within the scope of sound medical judgment, suitable for use in contact with cells and/or tissues without excessive toxicity, irritation, allergic response, or other problem or complication, commensurate with a reasonable benefit/risk ratio.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Each carrier must be “acceptable” in the sense of being compatible with the other ingredients of the formulation and not injurious to the cell or tissue being contacted. Additional ingredients that may be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

As is well known in the medical arts, the dosage for any one patient depends upon many factors, including the patient's size, weight, body surface area, age, the level of expression of inhibitory RNA expression required to achieve a therapeutic effect, stability of the inhibitory nucleic acid, specific disease being treated, stage of disease, sex, time and route of administration, general health, and other drugs being administered concurrently. In some embodiments, a rAAV particle as described herein is administered to a subject in an amount of about 1×10⁶ VG (viral genomes) to about 1×10¹⁶ VG per subject, or about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 2.1×10¹¹, 2.2×10¹¹, 2.3×10¹¹, 2.4×10¹¹, 2.5×10¹¹, 2.6×10¹¹, 2.7×10¹¹, 2.8×10¹¹, 2.9×10¹¹, 3×10¹¹, 4×10¹¹, 5×10¹¹, 6×10¹¹, 7×10¹¹, 7.1×10¹¹, 7.2×10¹¹, 7.3×10¹¹, 7.4×10¹¹, 7.5×10¹¹, 7.6×10¹¹, 7.7×10¹¹, 7.8×10¹¹, 7.9×10¹¹, 8×10¹¹, 9×10¹¹, 1×10¹², 1.1×10¹², 1.2×10¹², 1.3×10¹², 1.4×10¹², 1.5×10¹², 1.6×10¹², 1.7×10¹², 1.8×10¹², 1.9×10¹², 2×10¹², 3×10¹², 4×10¹², 4.1×10¹², 4.2×10¹², 4.3×10¹², 4.4×10¹², 4.5×10¹², 4.6×10¹², 4.7×10¹², 4.8×10¹², 4.9×10¹², 5×10¹², 6×10¹², 7×10¹², 8×10¹², 8.1×10¹², 8.2×10¹², 8.3×10¹², 8.4×10¹², 8.5×10¹², 8.6×10¹², 8.7×10¹², 8.8×10¹², 8.9×10¹², 9×10¹², 1×10¹³, 2×10¹³, 3×10¹³, 4×10¹³, 5×10¹³, 6×10¹³, 6.7×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG/subject. In some embodiments, a rAAV particle as described herein is administered to a subject in an amount of about 1×10⁶ VG/kg to about 1×10¹⁶ VG/kg, or about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 2.1×10¹¹, 2.2×10¹¹, 2.3×10¹¹, 2.4×10¹¹, 2.5×10¹¹, 2.6×10¹¹, 2.7×10¹¹, 2.8×10¹¹, 2.9×10¹¹, 3×10¹¹, 4×10¹¹, 5×10¹¹, 6×10¹¹, 7×10¹¹, 7.1×10¹¹, 7.2×10¹¹, 7.3×10¹¹, 7.4×10¹¹, 7.5×10¹¹, 7.6×10¹¹, 7.7×10¹¹, 7.8×10¹¹, 7.9×10¹¹, 8×10¹¹, 9×10¹¹, 1×10¹², 1.1×10¹², 1.2×10¹², 1.3×10¹², 1.4×10¹², 1.5×10¹², 1.6×10¹², 1.7×10¹², 1.8×10¹², 1.9×10¹², 2×10¹², 3×10¹², 4×10¹², 4.1×10¹², 4.2×10¹², 4.3×10¹², 4.4×10¹², 4.5×10¹², 4.6×10¹², 4.7×10¹², 4.8×10¹², 4.9×10¹², 5×10¹², 6×10¹², 7×10¹², 8×10¹², 8.1×10¹², 8.2×10¹², 8.3×10¹², 8.4×10¹², 8.5×10¹², 8.6×10¹², 8.7×10¹², 8.8×10¹², 8.9×10¹², 9×10¹², 1×10¹³, 2×10¹³, 3×10¹³, 4×10¹³, 5×10¹³, 6×10¹³, 6.7×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶VG/kg.

Pharmaceutical compositions may be administered in a manner appropriate to the disease or condition to be treated (or prevented) as determined by persons skilled in the medical art. An appropriate dose and a suitable duration and frequency of administration of the compositions will be determined by such factors as the health condition of the patient, size of the patient (i.e., weight, mass, or body area), the type and severity of the patient's disease, the particular form of the active ingredient, and the method of administration. In general, an appropriate dose and treatment regimen provide the composition(s) in an amount sufficient to provide therapeutic and/or prophylactic benefit (such as described herein, including an improved clinical outcome, such as more frequent complete or partial remissions, or longer disease-free and/or overall survival, or a lessening of symptom severity). For prophylactic use, a dose should be sufficient to prevent, delay the onset of, or diminish the severity of a disease associated with disease or disorder. Prophylactic benefit of the compositions administered according to the methods described herein can be determined by performing pre-clinical (including in vitro and in vivo animal studies) and clinical studies and analyzing data obtained therefrom by appropriate statistical, biological, and clinical methods and techniques, all of which can readily be practiced by a person skilled in the art.

Compositions (e.g., pharmaceutical compositions) may be administered by any route, including enteral (e.g., oral), parenteral, intravenous, intramuscular, intra-arterial, intramedullary, intrathecal, subpial, intraparenchymal, intrastriatal, intracranial, intracisternal, intra-cerebral, intracerebral ventricular, intraocular, intraventricular, intralumbar, subcutaneous, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. In general, the most appropriate route of administration will depend upon a variety of factors including the nature of the agent (e.g., its stability in the environment of the gastrointestinal tract), and/or the condition of the subject. In some embodiments, compositions are directly injected into the CNS of the subject. In some embodiments, direct injection into the CNS is intracerebral injection, intraparenchymal injection, intrathecal injection, intrastriatal injection, subpial injection, or any combination thereof. In some embodiments, direct injection into the CNS is direct injection into the cerebrospinal fluid (CSF) of the subject, optionally wherein the direct injection is is intracisternal injection, intraventricular injection, and/or intralumbar injection.

In some embodiments, pharmaceutical compositions comprising rAAV particles are formulated to reduce aggregation of rAAV particles, particularly where high rAAV particle concentrations are present (e.g., ^{˜}10¹³ VG/ml or more). Methods for reducing aggregation of rAAV particles are well known in the art and, include, for example, addition of surfactants, pH adjustment, salt concentration adjustment, etc. (See, e.g., Wright F R, et al., Molecular Therapy (2005) 12:171-178, incorporated herein by reference in its entirety).

### Kits

In some embodiments, the compositions provided herein may be assembled into pharmaceutical or research kits to facilitate their use in therapeutic or research use. A kit may include one or more containers comprising: (a) inhibitory nucleic acid, isolated nucleic acid comprising an expression construct, or vector as described herein; (b) instructions for use; and optionally (c) reagents for transducing the kit component (a) into a host cell. In some embodiments, the kit component (a) may be in a pharmaceutical formulation and dosage suitable for a particular use and mode of administration. For example, the kit component (a) may be presented in unit-dose or multi-dose containers, such as sealed ampoules or vials. The components of the kit may require mixing one or more components prior to use or may be prepared in a premixed state. The components of the kit may be in liquid or solid form, and may require addition of a solvent or further dilution. The components of the kit may be sterile. The instructions may be in written or electronic form and may be associated with the kit (e.g., written insert, CD, DVD) or provided via internet or web-based communication. The kit may be shipped and stored at a refrigerated or frozen temperature.

### Methods of Treatment

In another aspect, the present disclosure provides methods for inhibiting the expression or activity of ATXN2 in a cell, comprising administering a composition of the present disclosure (e.g., inhibitory nucleic acid, isolated nucleic acid comprising an expression construct encoding an inhibitory nucleic acid, vector, rAAV particle, pharmaceutical composition) to a cell, thereby inhibiting the expression or activity of ATXN2 in the cell. In some embodiments, the cell is a CNS cell. In some embodiments, the cell is a non-neuronal cell or neuronal cell of the CNS. In some embodiments, the non-neuronal cell of the CNS is a glial cell, astrocyte, or microglial cell. In some embodiments, the cell is in vitro. In some embodiments, the cell is from a subject having one or more symptoms of a neurodegenerative disease or suspected of having a neurodegenerative disease. In some embodiments, the cell expresses an ATXN2 having at least 22, 23, 24, 25, 26, 27, 28, 29, 30, 31, 32, 33, 34 or more CAG trinucleotide (polyglutamine) repeats. In some embodiments, the cell expresses an ATXN2 having about 22 or 23 repeats, 24-32 repeats, or 33-100 or more repeats.

In another aspect, the present disclosure provides methods for inhibiting the expression or activity of ATXN2 in the central nervous system of a subject, comprising administering a composition of the present disclosure (e.g., inhibitory nucleic acid, isolated nucleic acid comprising an expression construct encoding an inhibitory nucleic acid, vector, rAAV particle, pharmaceutical composition) to the subject, thereby inhibiting the expression or activity of ATXN2 in the subject.

In another aspect, the present disclosure provides methods for treating a subject having or suspected of having a neurodegenerative disease, comprising administering a composition of the present disclosure (e.g., inhibitory nucleic acid, isolated nucleic acid comprising an expression construct encoding an inhibitory nucleic acid, vector, rAAV particle, pharmaceutical composition) to the subject, thereby treating the subject. As used herein, the term “treat” refers to preventing or delaying onset of neurodegenerative disease (e.g., ALS/FTD, Alzheimer's disease, Parkinson's disease, etc.); reducing severity of neurodegenerative disease; reducing or preventing development of symptoms characteristic of neurodegenerative disease; preventing worsening of symptoms characteristic of neurodegenerative disease, or any combination thereof.

Neurodegenerative diseases that may be treated in a subject using the compositions of the present disclosure include neurodegenerative diseases where ATXN2 is a causative agent (e.g., SCA2), as well as neurodegenerative diseases where ATXN2 is not the causative agent but modifies TDP-43 pathological aggregation. Neurodegenerative diseases associated with TDP-43 proteinopathy include ALS, FTD, primary lateral sclerosis, progressive muscular atrophy, limbic-predominant age-related TDP-43 encephalopathy, chronic traumatic encephalopathy, dementia with Lewy bodies, corticobasal degeneration, progressive supranuclear palsy (PSP), dementia Parkinsonism ALS complex of guam (G-PDC), Pick's disease, hippocampal sclerosis, Huntington's disease, Parkinson's disease, and Alzheimer's disease.

In some embodiments, the subject is characterized as having an ATXN2 allele having at least 22, 23, 24, 25, 26, 27, 28, 29, 30, 31, 32, 33, 34 or more CAG trinucleotide (polyglutamine) repeats. In some embodiments, the subject is characterized as having an ATXN2 allele having about 22 or 23 repeats, 24-32 repeats, or 33-100 or more repeats.

In some embodiments, the methods for treatment of the present disclosure reduces, prevents, or slows development or progression of one or more symptom characteristic of a neurodegenerative disease. Examples of symptoms characteristic of neurodegenerative disease include motor dysfunction, cognitive dysfunction, emotional/behavioral dysfunction, or any combination thereof. Paralysis, shaking, unsteadiness, rigidity, twitching, muscle weakness, muscle cramping, muscle stiffness, muscle atrophy, difficulty swallowing, difficulty breathing, speech and language difficulties (e.g., slurred speech), slowness of movement, difficulty with walking, dementia, depression, anxiety, or any combination thereof.

In some embodiments, the methods for treatment of the present disclosure of the present disclosure comprise administration as a monotherapy or in combination with one or more additional therapies for the treatment of the neurodegenerative disease. Combination therapy may mean administration of the compositions of the present disclosure (e.g., inhibitory nucleic acid, isolated nucleic acid comprising an expression construct encoding an inhibitory nucleic acid, vector, rAAV particle, pharmaceutical composition) to the subject concurrently, prior to, subsequent to one or more additional therapies. Concurrent administration of combination therapy may mean that the the compositions of the present disclosure (e.g., inhibitory nucleic acid, isolated nucleic acid comprising an expression construct encoding an inhibitory nucleic acid, vector, rAAV particle, pharmaceutical composition) and additional therapy are formulated for administration in the same dosage form or administered in separate dosage forms.

In some embodiments, the one or additional therapies that may be used in combination with the inhibitory nucleic acids of the present disclosure include: inhibitory nucleic acids or antisense oligonucleotides that target neurodegenerative disease related genes or transcripts (e.g., C9ORF72), gene editing agents (e.g., CRISPR, TALEN, ZFN based systems) that target neurodegenerative related genes (e.g., C9ORF72), agents that reduce oxidative stress, such as free radical scavengers (e.g., Radicava (edaravone), bromocriptine); antiglutamate agents (e.g., Riluzole, Topiramate, Lamotrigine, Dextromethorphan, Gabapentin and AMPA receptor antagonist (e.g., Talampanel)); Anti-apoptosis agents (e.g., Minocycline, Sodium phenylbutyrate and Arimoclomol); Anti-inflammatory agents (e.g., ganglioside, Celecoxib, Cyclosporine, Nimesulide, Azathioprine, Cyclophosphamide, Plasmapheresis, Glatiramer acetate and thalidomide); Beta-lactam antibiotics (penicillin and its derivatives, ceftriaxone, and cephalosporin); Dopamine agonists (Pramipexole, Dexpramipexole); and neurotrophic factors (e.g., IGF-1, GDNF, BDNF, CTNF, VEGF, Colivelin, Xaliproden, Thyrotrophin-releasing hormone and ADNF).

In some embodiments, an inhibitory nucleic acid of the present disclosure is administered in combination with an additional therapy targeting C9ORF72. In some embodiments, the additional therapy targeting C9ORF72 comprises an inhibitory nucleic acid targeting C9ORF72 transcript, a C9ORF72 specific antisense oligonucleotide, or a C9ORF72 specific gene editing agent. Examples of C9ORF72 specific therapies are described in U.S. Pat. No. 9,963,699 (antisense oligonucleotides); PCT Publication No. WO2019/032612 (antisense oligonucleotides); U.S. Pat. No. 10,221,414 (antisense oligonucleotides); U.S. Pat. No. 10,407,678 (antisense oligonucleotides); U.S. Pat. No. 9,963,699 (antisense oligonucleotides); US Patent Publication US2019/0316126 (inhibitory nucleic acids); US Patent Publication No. 2019/0167815 (gene editing); PCT Publication No. WO2017/109757 (gene editing), each of which is incorporated by reference in its entirety.

In some embodiments, a subject treated in any of the methods described herein is a mammal (e.g., mouse, rat), preferably a primate (e.g., monkey, chimpanzee), or human.

In any of the methods of treatment described herein, a composition of the present disclosure (e.g., inhibitory nucleic acid, isolated nucleic acid comprising an expression construct encoding an inhibitory nucleic acid, vector, rAAV particle, pharmaceutical composition) may be administered to the subject by intrathecal, subpial, intraparenchymal, intrastriatal, intracranial, intracisternal, intra-cerebral, intracerebral ventricular, intraocular, intraventricular, intralumbar administration, or any combination thereof.

In some embodiments, a composition of the present disclosure (e.g., inhibitory nucleic acid, isolated nucleic acid comprising an expression construct encoding an inhibitory nucleic acid, vector, rAAV particle, pharmaceutical composition) is directly injected into the CNS of the subject. In some embodiments, direct injection into the CNS is intracerebral injection, intraparenchymal injection, intrathecal injection, intrastriatal injection, subpial injection, or any combination thereof. In some embodiments, direct injection into the CNS is direct injection into the cerebrospinal fluid (CSF) of the subject, optionally wherein the direct injection is intracisternal injection, intraventricular injection, intralumbar injection, or any combination thereof.

In some embodiments, the methods of the present disclosure reduces ATXN2 expression or activity in a cell by at least 10%, at least 15%, at least 20%, at least 25%, at least 30%, at least 35%, at least 40%, at least 45%, at least 50%, at least 60%, at least 70%, at least 80%, at least 90% at least 95% or more in a cell compared to the expression level of ATXN2 in a cell that has not been contacted with the inhibitory nucleic acid. In some embodiments, the methods of the present disclosure reduces ATXN2 expression or activity in a cell by 10-20%, 10-30%, 10-40%, 10-50%, 10-60%, 10-70%, 10-80%, 10-90%, 10-95%, 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% compared to the expression level of ATXN2 in a cell that has not been contacted with the inhibitory nucleic acid.

In some embodiments, the methods of the present disclosure reduces ATXN2 expression or activity in the CNS of a subject by at least 10%, at least 15%, at least 20%, at least 25%, at least 30%, at least 35%, at least 40%, at least 45%, at least 50%, at least 60%, at least 70%, at least 80%, at least 90% at least 95% or more in the CNS compared to the expression level of ATXN2 in the CNS of an untreated subject. In some embodiments, the methods of the present disclosure reduces ATXN2 expression or activity in the CNS of a subject by 10-20%, 10-30%, 10-40%, 10-50%, 10-60%, 10-70%, 10-80%, 10-90%, 10-95%, 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% compared to the expression level of ATXN2 in the CNS of an untreated subject.

### EXAMPLES

### Example 1: Design and Testing of siRNA Sequences to Knock Down Human ATAXIN-2

A number of criteria were used to select and design siRNA sequences to knock down ATXN2. The potential siRNA sequences that were initially considered included all possible 22-nucleotide RNAs complementary to ENST00000377617.7 (ATXN2-201). Human transcripts encoding for human Ataxin-2 were first examined. Only sequences found in all five of ATXN2 transcripts, NM_002973.3 (SEQ ID NO:2), ENST00000377617.7, ENST00000550104.5( ), ENST00000608853.5( ), and ENST00000616825.4( ), were selected.

The set of sequences was then filtered by cross-reactivity to the orthologous ATXN2 gene in rhesus and cynomolgous monkey. This allows the sequences to be tested in these species if needed to establish the activity and safety of gene therapies containing these inhibitory nucleic acid sequences prior to therapeutic use in humans. Thus, the sequence was also required to be in rhesus (Macaca mulatta) ATXN2 (NCBI Reference Sequences: XM_015152804.1, XM_015152805.1, XM_015152806.1, XM_015152807.1, XM_015152809.1, XM_015152810.1, XM_015152811.1, XM_015152812.1, XM_015152814.1, (Ensemble ID:) ENSMMUT00000062319, and ENSMMUT00000074794) and cynomolgous monkeys (Macaca fascicularis) ATXN2 (NCBI Reference Sequences: XM_005572266.2, XM_005572267.2, XM_015431532.1, XM_015431533.1, XM_015431534.1, XM_015431535.1, XM_015431536.1, XM_015431537.1, XM_015431538.1, XM_015431539.1, XM_015431540.1, XM_015431541.1, XM_015431542.1, XM_015431543.1, XM_015431544.1, XM_015431546.1, XM_015431547.1, XM_015431548.1, XM_015431549.1, XM_015431550.1, ENSMFAT00000019903.1). The ATXN2 transcript XM_015152813.1 of rhesus was also examined. This transcript was observed to be lacking a component of exon 1 and exon 2 (by comparison to human ATXN2 sequence SEQ ID NO:2). As described above for rhesus sequences, the following Macaca ATXN2 transcripts were identified to lack upstream sequence in exon 1: XM_015431551.1 and ENSMFAT00000019905.1. For these sequences, the exon 1 sequence was added back from human (SEQ ID NO:2) so as not to filter out that sequence. The nucleotide sequence in the ATXN2 gene encoding for the poly-glutamine repeat contains elements likely found elsewhere in the genome in other poly-glutamine repeat sequences. It is possible that automated transcript assignment algorithms, relying on alignment of RNAseq data, would mis-align sequencing reads overlapping with the poly-glutamine-encoding stretch (CAG repeating sequence) elsewhere in the genome, undercounting this sequence. These sequences in the upstream part of ATXN2 were therefore not excluded, except due to non-conservation from human to primate sequences.

Based on an analysis of brain RNAseq, exon 12 skipping is about 3% frequency, so this was not filtered out despite some alternative splice isoforms not including this isoform.

After defining the sequences expected to be present in human ATXN2 and key toxicology species, siRNAs were further selected based on criteria to reduce likelihood of off-target effects and to improve likelihood of strong ATXN2 knockdown. The seed sequences of both the antisense and sense strands of siRNAs, that is, bases 2-7 of the sequences which are known to be key determinants of activity of endogenous microRNAs, were examined for conservation in endogenous miRNAs expressed in human, mouse and rat. Antisense sequences present in any human endogenous miRNA were excluded, as were all sequences that were conserved in both mouse and rat. Sense sequences were excluded if seed regions were conserved in endogenous miRNAs present in more than 2 species out of human, mouse and rat.

A predicted knockdown ranking was calculated by adapting a version of an algorithm published in Pelossof et al. (Nature Biotechnology (2017) 35:350-353). Essentially, a support vector machine was trained on tiled sequencing data, provided in the publication. To generate the points in the space in which the support vector machine attempts to separate training examples which are labeled positive and negative, for good and bad knockdown respectively, features were selected as a weighted degree kernel. Features input to the support vector machine classifier were essentially the same as in Pelossof et al. For the SVM model, the “LibSVM” function from the Shogun module (version 6.1.3, Python version 2.7) was used instead of “SVMlite.”

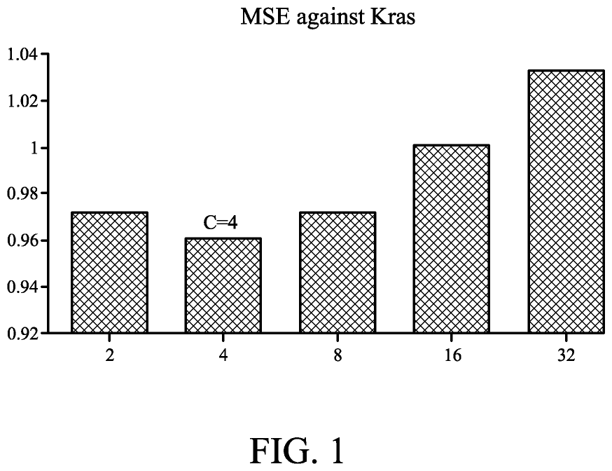

The training set included 18,421 shRNA sequences from the genes PCNA, Trp53, Hras, Rpa3, Mcl1, hMyc, Myc, Bcl2, and Kras, all from the ‘TILE’ data set included in Pelossof et al. The TILE dataset empirically tests the performance of unbiased libraries of shRNAs covering sequences in the 9 genes described. The cost function c was assessed across a range of values training the SVM classifier on all genes except one of the nine left out, and calculating mean squared error on predictions for performance on data from the held-out gene. An example with Kras as the held out gene is shown (FIG. 1). A value of c=4 was selected which minimized the mean square error among values of c tested.

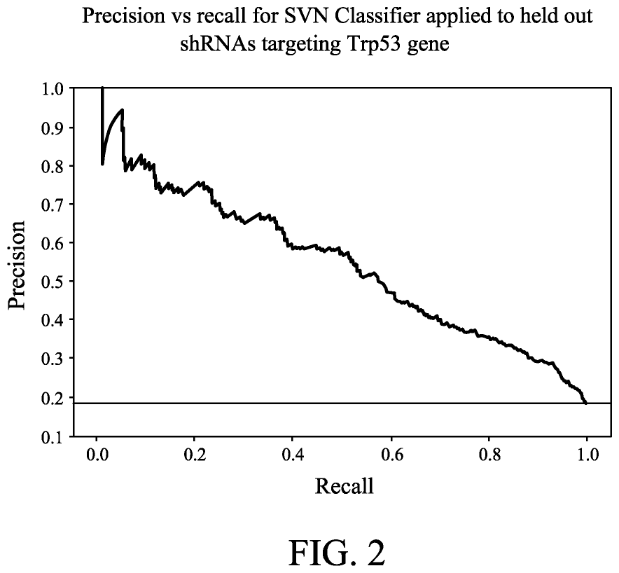

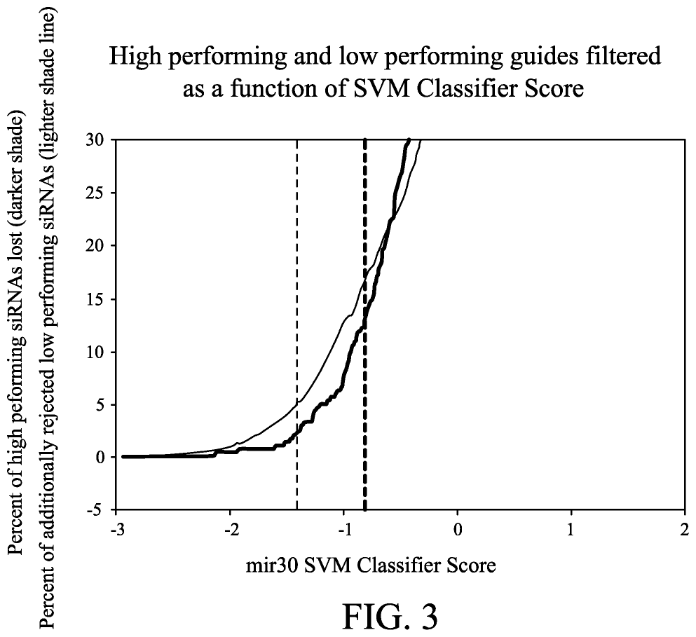

To further assess the performance of the classifier, knockdown data from another gene in the data set (Trp53) was held out after training the classifier on the other 8 genes. FIG. 2 shows a precision-recall curve for the classifier, as trained on data not including the Trp53 shRNAs, predicting performance of shRNA knockdown in the Trp53 targeting shRNAs. That is, after filtering shRNAs by a given classifier score, the fraction of of true positives identified by the classifier (recall) is plotted as a function of the number of true positives versus false positives (precision) (FIG. 2). Additionally, the anticipated cumulative fraction of ‘positive’ shRNAs (high performing) shRNAs that are expected to be lost as the classifier score was increased in stringency was plotted (FIG. 3), alongside the percent improvement in rejection of low-performing shRNAs. A separation in the curves was noted between scores of approximately −1.5 to −0.8, going from roughly the 25^{th} to 50^{th} percentiles of scores for Trp53 targeting shRNAs.

Next, siRNA sequences were triaged by specificity considerations, then ranked by the score from the above classifier. In addition to conservation of the seed sequences with endogenous miRNAs, as described above, metrics of specificity were: (a) comparison of seed sequences (guide bases 2-7) to a published data set of transfected siRNA seed sequences versus cell proliferation (Gaoao et al. Nature Communications (2018) 9:4504), excluding sequences with a >70% reduction of cell proliferation in the published assay; (b) the number of transcripts complementary to the first 19 nucleotides of the guide sequence, with 2 or fewer mismatches, was required to be less than 15; and (c) other considerations such as an internal algorithm of specificity were also factored in but triaged fewer samples than the criteria of (a) and (b).

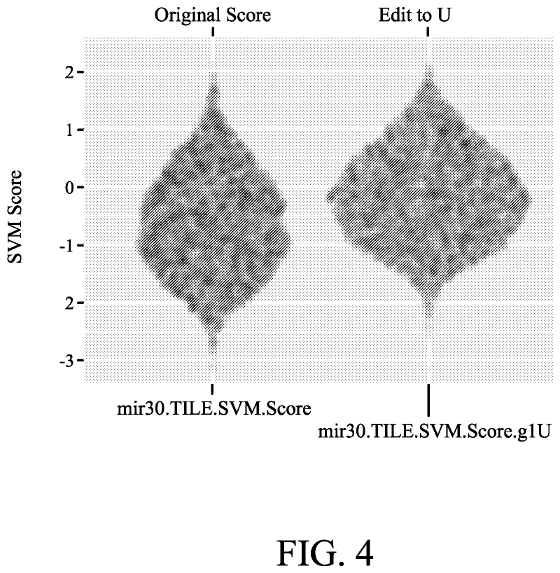

Following filtering by specificity, sequences in the most common ATXN2 transcript, were ranked by SVM score and top-ranked candidate sequences selected. In calculating the SVM classifier score for shRNAs, however, it was found that the classifier score significantly increased for shRNAs beginning with U (FIG. 4). This was consistent with prior prediction algorithms (e.g., Vert et al., BMC Bioinformatics (2006) BMC Bioinformatics 7:520) and literature suggesting that the argonaute 2 binding pocket interacts best with this base, although guide base 1 does not base pair with the target mRNA (Boland et al., EMBO Reports (2010) 11:522-527). Therefore, for shRNA design, if the base was a ‘G’ or ‘C,’ based on complementarity to the target mRNA sequence, that base was replaced with a ‘U’ and the corresponding performance score calculated. The top 93 sequences beginning with A or U (SVM score >−0.8) and 34 sequences edited from a shRNA beginning with G or C, with a more stringent filter (SVM score >0.4).

Additional sequences were included for testing based on other criteria, including: (a) cross-reactivity with ATXN2L. ATXN2L shares considerable amino acid sequence similarity with ATXN2. Homologous genes often execute similar functions in a cell, and it is possible that knockdown of ATXN2L may serve similar therapeutic functions as knocking down ATXN2. Sequences which match both ATXN2 and ATXN2L may therefore have additional therapeutic benefit, and thus, 10 sequences were selected with potential to target both ATXN2 and ATXN2L; (b) sequences meeting a stringent off-target match criteria, with 2 or fewer transcripts matching at 2 or fewer positions in the first 19 nucleotides of the siRNA guide sequence (10 siRNAs), but ignoring SVM-based efficacy prediction; (c) sequences with perfect match or single mismatch to mouse ATXN2 in the first 19 nucleotides of the guide sequence. ‘Single mismatch’ guide sequences were defined as those where only one mismatch occurs between bases 12 and 19 nts against the mouse sequence, and none in bases 1-11. For guide sequences perfect-matching or single-mismatching mouse, the specificity criteria were relaxed, with guide sequences accepted with fewer than 50 complementary transcripts with 2 or fewer mismatches.

Selection of Cell Line to Screen siRNA Candidates

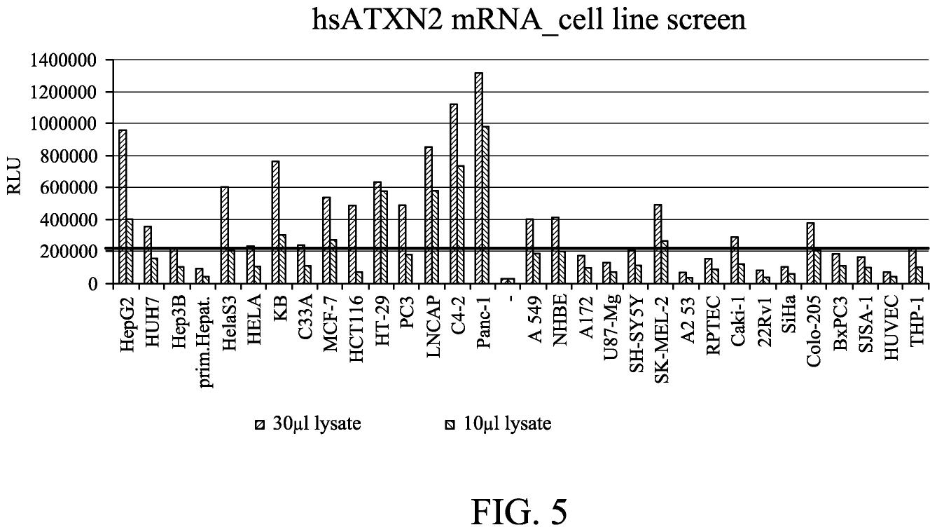

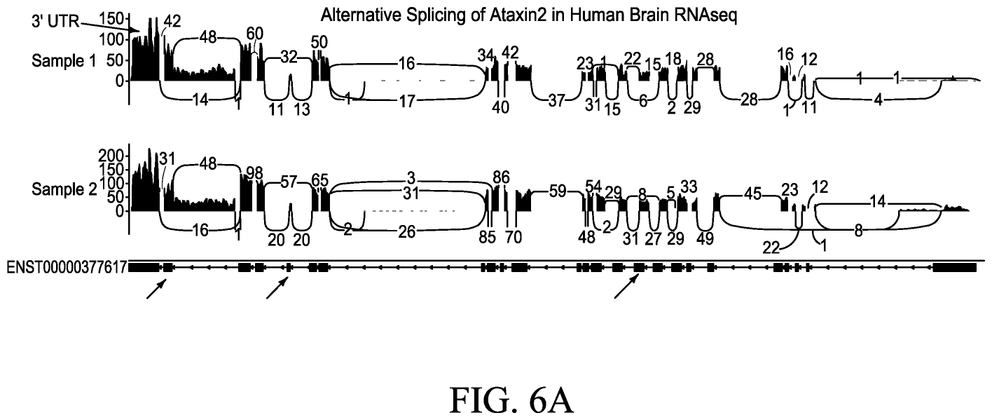

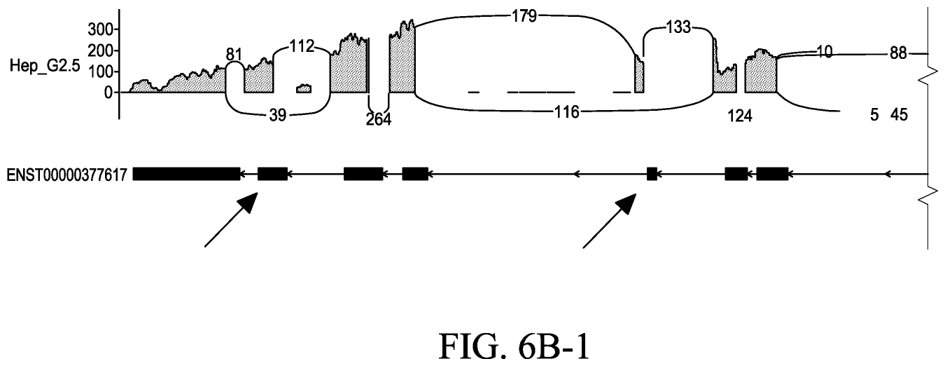

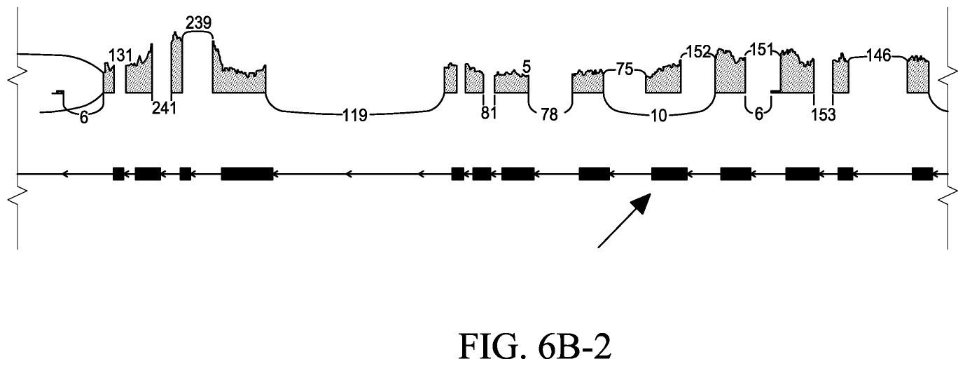

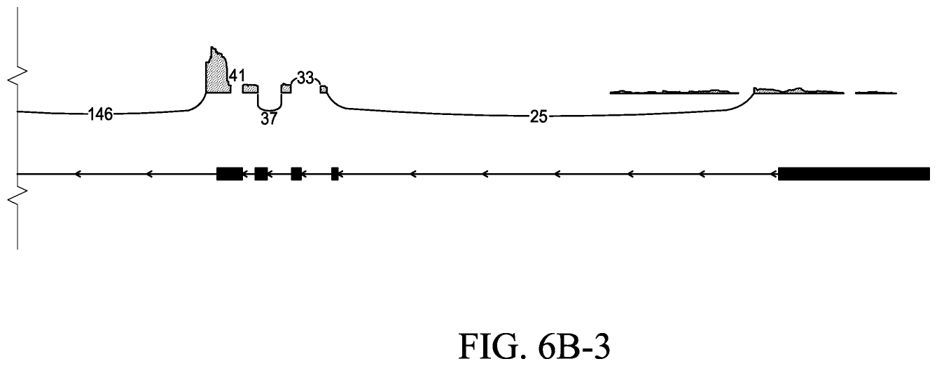

Following selection of siRNAs for testing, an in vitro cell system was established to assess knockdown of ATXN2 by siRNAs. ATXN2 levels were assessed by quantigene assay (Thermo Fisher), across a panel of cell lines (FIG. 5). The cell lines HepG2, KB, HT-29, LNCAP, C4-2 and Panc-1 all showed robust ATXN2 expression. To see if the splice patterns of cells were similar to that of ATXN2 in relevant target tissues, including in neurodegenerative disease states, RNAseq of postmortem human brain (Mayo Clinic Alzheimer's Disease Genetics Studies**; accessed via the synapse.org platform) was examined for splice patterns of ATXN2 and compared to data from cell lines (National Cancer Institute GDC Legacy Archive). In FIG. 6A, alternatively spliced exons were identified by reads crossing genomic regions that skip over the alternatively spliced exons. Exons 10, 21, and 24 in brain are frequently alternatively spliced. Examining alternative splicing in cell lines, HepG2 were similar to human brain (FIG. 6B). This line was selected for ATXN2 siRNA studies because of the high level of ATXN2 expression relative to background and consistent alternative splice patterns.

With regard to the synapse.org platform, study data were provided by the following sources: The Mayo Clinic Alzheimer's Disease Genetic Studies, led by Dr. Nilüfer Ertekin-Taner and Dr. Steven G. Younkin, Mayo Clinic, Jacksonville, Fla. using samples from the Mayo Clinic Study of Aging, the Mayo Clinic Alzheimer's Disease Research Center, and the Mayo Clinic Brain Bank. Data collection was supported through funding by NIA grants P50 AG016574, R01 AG032990, U01 AG046139, R01 AG018023, U01 AG006576, U01 AG006786, R01 AG025711, R01 AG017216, R01 AG003949, NINDS grant R01 NS080820, CurePSP Foundation, and support from Mayo Foundation. The following publications are applicable:
[1] Carrasquillo et. al., Nat Genet. (2009) 41:192-8.
[2] Zou et. al. PLoS Genet. (2012) 8(6):e1002707. [3] Allen et al. Sci Data. (2016) 3:160089.
   Synthesis and Testing of siRNAs

siRNAs were synthesized as 22 nucleotide RNAs, with 20 bp of complementarity (complementarity from positions 1-20, of guide and passenger strands). Here, guide strand refers to the sequence complementary to, or antisense to, the ATXN2 target mRNA, and passenger strand refers to the strand complementary to guide strand. Guide and passenger strands, also referred to as antisense and sense strand RNAs, are shown in Table 1. Sequences were synthesized as guide and passenger strands. All but 6 of the sequences met the following criteria: single strands within 0.05% of calculated mass (by LC/MS). At least 85% of full-length oligonucleotide purity (by HPLC). After annealing guide and passenger strands, duplex purity of >90% by non-denaturing HPLC. Oligonucleotides not meeting these criteria are noted as “FAIL,” but data are included for completeness.

Annealed siRNAs were reverse transfected, adding 20,000 cells per well of a 96-well plate, on top of a solution of lipofectamine 2000 with siRNA to yield a final siRNA concentration in the diluted culture media as noted below, in a volume of 0.5 microliters of transfection solution per well. siRNAs were tested in quadruplicate wells and incubated for 24 hours. ATXN2 and GAPDH levels were assayed in cell lysates by Quantigene assay using ATXN2 and GAPDH probes (Thermo Fisher). The ratio of ATXN2 mRNA levels to levels of the housekeeping gene GAPDH was calculated, and values were normalized to ATXN2/GAPDH ratios obtained for cells mock-treated with lipofectamine not containing siRNA.

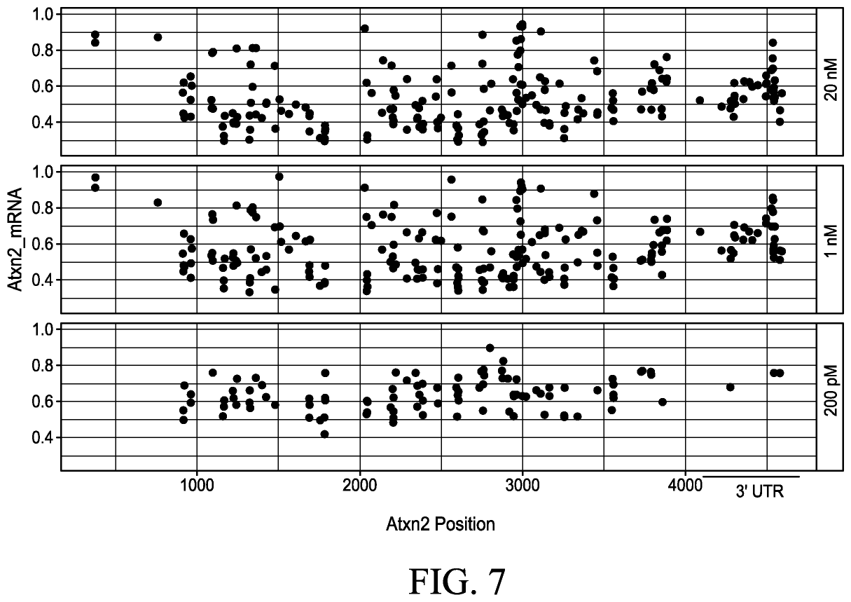

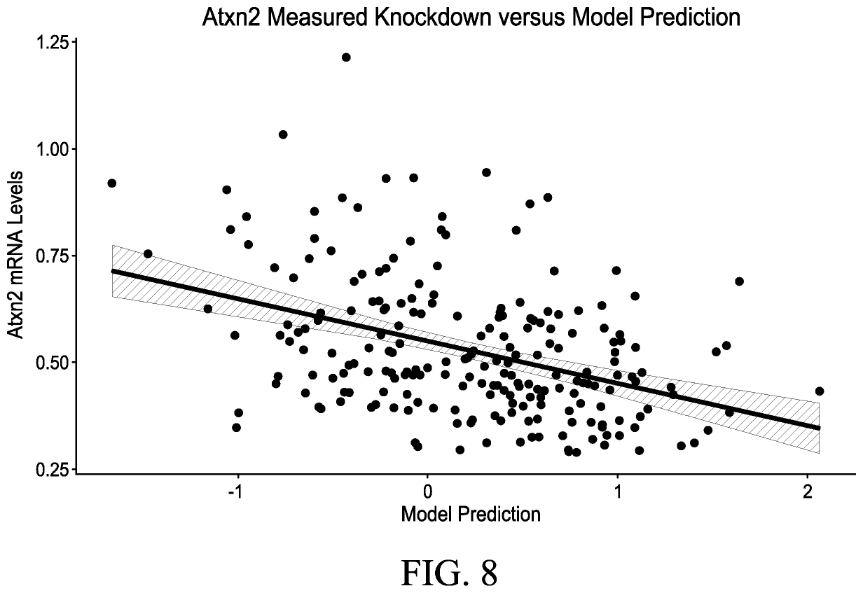

All siRNAs were tested at doses of 20 nM or 1 nM (final calculated concentration of siRNA in cell culture media) for level of ATXN2 following knockdown (Table 4). A significant correlation, as assessed by a linear model fit, was observed plotting the predicted SVM score classifier against the 20 nM siRNA knockdown data (FIG. 8) (p<10⁻⁸, R²=0.15). Subsequently, the top ranked 100 siRNAs, by ATXN2 knockdown from 1 nM siRNA dosing data, were rescreened at 200 pM (Table 5). FIG. 7 plots the knockdown of ATXN2 mRNA for siRNAs as a function for position along the ATXN2 transcript that they transcript.

TABLE 4
siRNA Single Point Testing Data 20 nM-knockdown of ATXN2 mRNA

| siRNA Duplex ID | 20 nM_mean | 20 nM_SD | 1 nM_mean | 1 nM_SD |
|---|---|---|---|---|
| XD-14738 | 84.1 | 6.8 | 91.1 | 3.6 |
| XD-14739 | 88.6 | 4.7 | 96.8 | 6.9 |
| XD-14740 | 87.1 | 3.4 | 82.9 | 3.4 |
| XD-14741 | 56.4 | 2.3 | 54.6 | 3.2 |
| XD-14742 | 44.5 | 2.5 | 44.7 | 3.5 |
| XD-14743 | 44.9 | 2 | 48.1 | 3.5 |
| XD-14744 | 62 | 2.9 | 65.7 | 2.1 |
| XD-14745 | 42.5 | 3.9 | 47.3 | 3.5 |
| XD-14746 | 65.4 | 3.9 | 62.5 | 2 |
| XD-14747 | 43.2 | 2.4 | 41.2 | 1.8 |
| XD-14748 | 52.4 | 1.6 | 49.3 | 1.9 |
| XD-14749 | 60.2 | 3 | 57.4 | 1.3 |
| XD-14750 | 52.3 | 3.3 | 53.5 | 1.2 |
| XD-14751 | 47.7 | 4 | 54.6 | 1.1 |
| XD-14752 | 78.4 | 3.3 | 76.4 | 6.4 |
| XD-14753 | 47.7 | 2.4 | 55 | 1.1 |
| XD-14754 | 47.5 | 8.4 | 50.6 | 2.8 |
| XD-14755 | 79 | 3.6 | 73.3 | 3.9 |
| XD-14756 | 37.6 | 5.2 | 46.8 | 7.8 |
| XD-14757 | 32.5 | 1 | 39.7 | 0.4 |
| XD-14758 | 29.6 | 0.9 | 35.3 | 5.2 |
| XD-14759 | 43.6 | 4.3 | 51.8 | 5.5 |
| XD-14760 | 45 | 1.3 | 52.3 | 3.6 |
| XD-14761 | 40.2 | 1.8 | 54.7 | 4.1 |
| XD-14762 | 39.4 | 2.5 | 47.8 | 2.3 |
| XD-14763 | 39.2 | 3 | 50.6 | 1.7 |
| XD-14764 | 80.9 | 4.4 | 81.2 | 8.7 |
| XD-14765 | 43.1 | 2.9 | 49.5 | 5.1 |
| XD-14766 | 30.3 | 0.7 | 33.1 | 3.6 |
| XD-14767 | 43.6 | 5.6 | 38.5 | 2.5 |
| XD-14768 | 35.9 | 3.3 | 40.9 | 2 |
| XD-14769 | 72 | 7.6 | 78.7 | 2 |
| XD-14770 | 50.7 | 7.6 | 56.9 | 3.1 |
| XD-14771 | 59.8 | 6.5 | 77.4 | 17.7 |
| XD-14772 | 81.1 | 2.1 | 80.2 | 1.1 |
| XD-14773 | 44.2 | 3.1 | 52.1 | 5.2 |
| XD-14774 | 81 | 7.1 | 74.8 | 12.1 |
| XD-14775 | 42.3 | 4.3 | 44.3 | 2.6 |
| XD-14776 | 50.3 | 2.2 | 45.7 | 2.7 |
| XD-14777 | 51 | 5.4 | 53.2 | 1.9 |
| XD-14778 | 71.2 | 5.2 | 69.2 | 6.1 |
| XD-14779 | 36.5 | 2.8 | 34.7 | 4.3 |
| XD-14780 | 121.4 | 8.7 | 97.2 | 6 |
| XD-14781 | 52.7 | 5.9 | 69.7 | 3.3 |
| XD-14782 | 46.3 | 2 | 61.1 | 3.2 |
| XD-14783 | 44.6 | 2.1 | 56.9 | 2.8 |
| XD-14784 | 49.8 | 1.3 | 64.4 | 2.7 |
| XD-14785 | 48.1 | 8.7 | 61.4 | 4.5 |
| XD-14786 | 45.1 | 2.5 | 44.7 | 6.5 |
| XD-14787 | 43.3 | 7.4 | 48.2 | 2.8 |
| XD-14788 | 34.8 | 1.4 | 41.7 | 4.7 |
| XD-14789 | 44.6 | 0.8 | 62.3 | 2.8 |
| XD-14790 | 31.3 | 0.8 | 36.7 | 2.6 |
| XD-14791 | 29.5 | 1.5 | 38.9 | 2.9 |
| XD-14792 | 31.2 | 2.6 | 38 | 0.9 |
| XD-14793 | 38.3 | 1.7 | 47.9 | 2.9 |
| XD-14794 | 34.7 | 1.4 | 38.6 | 1.3 |
| XD-14795 | 36.4 | 5.4 | 47.9 | 4.1 |
| XD-14796 | 91.9 | 7 | 91.1 | 8.3 |
| XD-14797 | 61.9 | 1.2 | 74.9 | 17.3 |
| XD-14798 | 31.2 | 2.3 | 33.9 | 2 |
| XD-14799 | 32.5 | 2.1 | 39.8 | 3.3 |
| XD-14800 | 30.4 | 1.4 | 43.3 | 6 |
| XD-14801 | 32.8 | 3.4 | 36.2 | 0.9 |
| XD-14802 | 56.3 | 1 | 70.5 | 2.4 |
| XD-14803 | 45.2 | 1.3 | 56.8 | 5.2 |
| XD-14804 | 74.3 | 3.6 | 76.2 | 5 |
| XD-14805 | 47.2 | 3.5 | 50.1 | 3.5 |
| XD-14806 | 71.4 | 5.8 | 75 | 4.4 |
| XD-14807 | 42.4 | 2.4 | 52.7 | 3.6 |
| XD-14808 | 42.8 | 2.6 | 53.1 | 5 |
| XD-14809 | 47.4 | 3.3 | 49 | 2.8 |
| XD-14810 | 39.7 | 1.7 | 48 | 1.4 |
| XD-14811 | 38.9 | 4.8 | 46.3 | 0.9 |
| XD-14812 | 40.8 | 4.9 | 47.1 | 2.3 |
| XD-14813 | 57.8 | 5.9 | 59.5 | 2.3 |
| XD-14814 | 103.2 | 29.6 | 81.5 | 2.9 |
| XD-14815 | 54. | 4.4 | 48.6 | 7 |
| XD-14816 | 35.9 | 2.8 | 40.8 | 3.3 |
| XD-14817 | 64 | 11.2 | 66.3 | 1.4 |
| XD-14818 | 49.4 | 2.8 | 49.5 | 1 |
| XD-14819 | 41.9 | 4 | 40.8 | 1.5 |
| XD-14820 | 42.8 | 4.3 | 46 | 1.3 |
| XD-14821 | 47.9 | 3.4 | 63 | 4.3 |
| XD-14822 | 37.3 | 3.1 | 45.5 | 2.9 |
| XD-14823 | 51.7 | 4.6 | 66.4 | 0.4 |
| XD-14824 | 38.6 | 1.3 | 45.5 | 2.3 |
| XD-14825 | 35.9 | 2.7 | 41.3 | 0.9 |
| XD-14826 | 39.2 | 1.5 | 46.1 | 3 |
| XD-14827 | 54.3 5. | 2.7 | 62.3 | 1.4 |
| XD-14828 | 63.8 | 1.9 | 77.1 | 3.5 |
| XD-14829 | 40.1 | 0.6 | 38.2 | 8.1 |
| XD-14830 | 36.7 | 0.7 | 46 | 0.9 |
| XD-14831 | 42.5 | 2.9 | 61.8 | 3.4 |
| XD-14832 | 71.3 | 2 | 95.6 | 1.5 |
| XD-14833 | 56.5 | 2.8 | 75.1 | 2.3 |
| XD-14834 | 38.3 | 1.6 | 47 | 2.6 |
| XD-14835 | 30.6 | 2.4 | 38.5 | 1.6 |
| XD-14836 | 44.5 | 3.2 | 58 | 2.7 |
| XD-14837 | 29.2 | 3.4 | 36.2 | 0.5 |
| XD-14838 | 36.5 | 2 | 46.1 | 1.2 |
| XD-14839 | 32 | 2.9 | 42.5 | 4 |
| XD-14840 | 29.3 | 2.3 | 34.1 | 7.6 |
| XD-14841 | 32.9 | 2.5 | 41.2 | 3.7 |
| XD-14842 | 38.8 | 3.4 | 45.6 | 2.5 |
| XD-14843 | 32.9 | 1.2 | 40 | 1.6 |
| XD-14844 | 88.5 | 4.9 | 84.6 | 4.8 |
| XD-14845 | 72.5 | 9.4 | 67.6 | 1.9 |
| XD-14846 | 29 | 1.7 | 34.6 | 3.4 |
| XD-14847 | 34.1 | 1.2 | 38.6 | 2.5 |
| XD-14848 | 40.4 | 0.5 | 46.3 | 3 |
| XD-14849 | 58.5 | 2.1 | 67 | 1.4 |
| XD-14850 | 34.7 | 0.6 | 38.7 | 0.7 |
| XD-14851 | 46.6 | 3.4 | 47 | 0.9 |
| XD-14852 | 61.4 | 2.4 | 55.9 | 2.6 |
| XD-14853 | 47 | 3.7 | 42 | 2.4 |
| XD-14854 | 43.3 | 5.3 | 40.8 | 4.9 |
| XD-14855 | 45.5 | 1.6 | 43.7 | 2.1 |
| XD-14856 | 43.9 | 3.3 | 40.7 | 5 |
| XD-14857 | 39.5 | 5.3 | 36.1 | 2.3 |
| XD-14858 | 63.8 | 4.7 | 54.1 | 1.1 |
| XD-14859 | 39 | 3.6 | 40.3 | 1 |
| XD-14860 | 35.5 | 3.2 | 36.2 | 1.6 |
| XD-14861 | 35.8 | 2.9 | 42.3 | 2.2 |
| XD-14862 | 85.3 | 2.6 | 84.2 | 7 |
| XD-14863 | 56.3 | 6.3 | 52.6 | 3.4 |
| XD-14864 | 46.3 | 3.1 | 47.2 | 2.3 |
| XD-14865 | 77.5 | 2.6 | 79.6 | 4 |
| XD-14866 | 52.6 | 4.4 | 53.2 | 7.4 |
| XD-14867 | 70.6 | 4.6 | 56.7 | 5.4 |
| XD-14868 | 79.8 | 3.4 | 72.5 | 3.8 |
| XD-14869 | 93.1 | 3 | 89.4 | 3.7 |
| XD-14870 | 60.9 | 0.9 | 65.1 | 4.2 |
| XD-14871 | 86.1 | 1.6 | 94 | 8.6 |
| XD-14872 | 93.1 | 3.6 | 91.8 | 5.5 |
| XD-14873 | 94.4 | 2.3 | 90.2 | 4.6 |
| XD-14874 | 60.7 | 4.3 | 56.9 | 7.5 |
| XD-14875 | 50 | 1.3 | 49.5 | 2.7 |
| XD-14876 | 53.5 | 15.9 | 51.7 | 4.1 |
| XD-14877 | 55 | 4 | 61 | 1.4 |
| XD-14878 | 49.6 | 1.5 | 47.4 | 2.2 |
| XD-14879 | 65 | 2.2 | 64.9 | 2.3 |
| XD-14880 | 47 | 1 | 44.5 | 1.8 |
| XD-14881 | 90.3 | 4.7 | 90.6 | 2 |
| XD-14882 | 39.7 | 1.5 | 40.1 | 5.1 |
| XD-14883 | 58 | 1.4 | 65.9 | 5 |
| XD-14884 | 62.7 | 2.2 | 67.9 | 6.1 |
| XD-14885 | 46.6 | 3.3 | 53.8 | 6.6 |
| XD-14886 | 39.5 | 1.4 | 44.4 | 7.5 |
| XD-14887 | 38.2 | 0.4 | 41.7 | 5.1 |
| XD-14888 | 61.4 | 2.1 | 68.9 | 4.9 |
| XD-14889 | 31.2 | 8.8 | 40.7 | 2.7 |
| XD-14890 | 36.3 | 1.9 | 37.3 | 2.7 |
| XD-14891 | 45.2 | 4 | 46.8 | 2.1 |
| XD-14892 | 49.1 | 2.8 | 62.5 | 1.7 |
| XD-14893 | 41.7 | 2.3 | 49.7 | 6 |
| XD-14894 | 47 | 2.7 | 64.9 | 3 |
| XD-14895 | 53.3 | 4 | 67.2 | 2.3 |
| XD-14896 | 45.1 | 1.3 | 66.8 | 2.4 |
| XD-14897 | 74.3 | 8.9 | 87.7 | 4.6 |
| XD-14898 | 68.4 | 5.8 | 73 | 4.8 |
| XD-14899 | 44.3 | 6.4 | 55 | 1.2 |
| XD-14900 | 45.6 | 2.8 | 47.8 | 1.6 |
| XD-14901 | 47.9 | 12.1 | 41.4 | 1.7 |
| XD-14902 | 56.1 | 6.3 | 52.8 | 2.5 |
| XD-14903 | 52.1 | 4.2 | 46.6 | 2.7 |
| XD-14904 | 47 | 3.7 | 36.6 | 8.4 |
| XD-14905 | 40.6 | 2 | 40.6 | 3.2 |
| XD-14906 | 47.1 | 4.6 | 50.7 | 3.3 |
| XD-14907 | 57 | 4.1 | 51.1 | 2.7 |
| XD-14908 | 58.8 | 6.3 | 51.1 | 4 |
| XD-14909 | 47.1 | 2.8 | 50 | 2.6 |
| XD-14910 | 46.9 | 2.8 | 53.5 | 9.2 |
| XD-14911 | 61.7 | 3.6 | 55 | 4.3 |
| XD-14912 | 58 | 3.8 | 59.3 | 3.9 |
| XD-14913 | 72.1 | 5.5 | 73.3 | 6.4 |
| XD-14914 | 68.9 | 4.7 | 66.3 | 5.7 |
| XD-14915 | 47.4 | 5.1 | 59.1 | 9.5 |
| XD-14916 | 43.1 | 5.7 | 55.6 | 9.4 |
| XD-14917 | 43.4 | 4.6 | 42.7 | 4.3 |
| XD-14918 | 64.2 | 2.3 | 67.4 | 5.7 |
| XD-14919 | 62.5 | 6.6 | 61.8 | 3.8 |
| XD-14920 | 76.1 | 1.8 | 67.6 | 4.2 |
| XD-14921 | 64.4 | 3.9 | 73.9 | 7 |
| XD-14922 | 52.2 | 6.9 | 66.7 | 7.5 |
| XD-14923 | 48.7 | 2.6 | 56.3 | 3.8 |
| XD-14924 | 47.6 | 1 | 51.8 | 2.1 |
| XD-14925 | 51.7 | 3.7 | 56.5 | 6.2 |
| XD-14926 | 43 | 3.9 | 54.9 | 3.2 |
| XD-14927 | 53.3 | 6.2 | 64.3 | 8.2 |
| XD-14928 | 61.7 | 1.7 | 70.4 | 3.8 |
| XD-14929 | 54.7 | 3.9 | 64.4 | 2.8 |
| XD-14930 | 50.1 | 3 | 63.6 | 5.5 |
| XD-14931 | 52.9 | 3.9 | 62.4 | 4.9 |
| XD-14932 | 62.6 | 3.7 | 69 | 3.8 |
| XD-14933 | 62.2 | 5 | 66.7 | 3.3 |
| XD-14934 | 59.7 | 7.6 | 62 | 4.8 |
| XD-14935 | 60.5 | 4.1 | 65.9 | 7.5 |
| XD-14936 | 54.4 | 2.6 | 71.3 | 4.7 |
| XD-14937 | 61.2 | 5.5 | 74.1 | 2.9 |
| XD-14938 | 65.8 | 6.6 | 71.9 | 3.7 |
| XD-14939 | 61.9 | 7.8 | 73.8 | 2.6 |
| XD-14940 | 68.8 | 6.8 | 79.6 | 2.2 |
| XD-14941 | 57.8 | 4.1 | 69.7 | 3.1 |
| XD-14942 | 75.3 | 9.3 | 85.4 | 4.4 |
| XD-14943 | 69.8 | 4.2 | 77.7 | 6.7 |
| XD-14944 | 84.1 | 4.3 | 84.2 | 8.5 |
| XD-14945 | 53.5 | 3 | 55.2 | 2 |
| XD-14946 | 58 | 1.8 | 56.9 | 3.7 |
| XD-14947 | 59.2 | 2.2 | 59.2 | 1.3 |
| XD-14948 | 56.8 | 4.4 | 53.4 | 2.4 |
| XD-14949 | 51.7 | 2.5 | 52.4 | 2.3 |
| XD-14950 | 63.2 | 2.2 | 69.6 | 4.3 |
| XD-14951 | 53.9 | 2.7 | 62.6 | 3.8 |
| XD-14952 | 40.3 | 0.9 | 51.3 | 6.2 |
| XD-14953 | 46.6 | 2.5 | 56.1 | 4.1 |
| XD-14954 | 56.1 | 8.6 | 55.9 | 6.6 |

TABLE 5
siRNA Single Point Testing Data 200 pM

| siRNA Duplex ID | 0.2 nM_mean | 0.2 nM_SD |
|---|---|---|
| XD-14742 | 55.2 | 5.4 |
| XD-14743 | 49.9 | 3.2 |
| XD-14745 | 68.9 | 2.5 |
| XD-14747 | 59.3 | 5.8 |
| XD-14748 | 63.9 | 3.8 |
| XD-14754 | 75.9 | 3.2 |
| XD-14756 | 52 | 8.9 |
| XD-14757 | 57.2 | 6.7 |
| XD-14758 | 60.8 | 3.3 |
| XD-14759 | 60.3 | 3.4 |
| XD-14760 | 65.8 | 4.2 |
| XD-14762 | 61.7 | 7.7 |
| XD-14763 | 58.1 | 5.1 |
| XD-14765 | 72.6 | 6 |
| XD-14766 | 59.5 | 4.1 |
| XD-14767 | 66.2 | 3.5 |
| XD-14768 | 56.5 | 3.7 |
| XD-14773 | 73.1 | 4.5 |
| XD-14775 | 69.1 | 5.1 |
| XD-14776 | 62.5 | 1.8 |
| XD-14779 | 58.2 | 1.3 |
| XD-14786 | 51.1 | 1.4 |
| XD-14787 | 61.7 | 1.5 |
| XD-14788 | 58.1 | 4.7 |
| XD-14790 | 49.7 | 1 |
| XD-14791 | 51.2 | 4.3 |
| XD-14792 | 41.9 | 4.6 |
| XD-14793 | 62.2 | 8.4 |
| XD-14794 | 60.6 | 5.3 |
| XD-14795 | 75.9 | 7.3 |
| XD-14798 | 53.1 | 2.4 |
| XD-14799 | 60.4 | 2.2 |
| XD-14800 | 54.1 | 4 |
| XD-14801 | 59.7 | 13.7 |
| XD-14805 | 56.7 | 2.4 |
| XD-14807 | 66.9 | 7 |
| XD-14809 | 48.4 | 2.9 |
| XD-14810 | 54.5 | 4.6 |
| XD-14811 | 51.1 | 3.5 |
| XD-14812 | 62.3 | 6.6 |
| XD-14815 | 76.1 | 2 |
| XD-14816 | 71.8 | 1.7 |
| XD-14818 | 75.9 | 1.9 |
| XD-14819 | 57.2 | 2.6 |
| XD-14820 | 68.9 | 2.7 |
| XD-14822 | 63.7 | 0.7 |
| XD-14824 | 69.8 | 3.6 |
| XD-14825 | 52.5 | 2.2 |
| XD-14826 | 60.6 | 3.6 |
| XD-14829 | 67.7 | 4.5 |
| XD-14830 | 59 | 5.7 |
| XD-14834 | 67.7 | 2.8 |
| XD-14835 | 51.7 | 12.4 |
| XD-14837 | 63.4 | 10.1 |
| XD-14838 | 73.2 | 4 |
| XD-14839 | 65.7 | 1.9 |
| XD-14840 | 60.7 | 2.7 |
| XD-14841 | 65.5 | 2.2 |
| XD-14842 | 67.7 | 2 |
| XD-14843 | 76.7 | 9.6 |
| XD-14846 | 54.9 | 6.5 |
| XD-14847 | 69.6 | 1.7 |
| XD-14848 | 77.5 | 1.1 |
| XD-14850 | 74.4 | 3.2 |
| XD-14851 | 89.7 | 2.4 |
| XD-14853 | 77 | 3.2 |
| XD-14854 | 73 | 3.6 |
| XD-14855 | 82.4 | 4.2 |
| XD-14856 | 72.7 | 4.7 |
| XD-14857 | 54.4 | 3.6 |
| XD-14859 | 63.8 | 3.2 |
| XD-14860 | 52 | 2.3 |
| XD-14861 | 63.2 | 3.5 |
| XD-14863 | 72.3 | 3.1 |
| XD-14864 | 63.9 | 2.2 |
| XD-14875 | 63.1 | 2.9 |
| XD-14876 | 62.9 | 2.5 |
| XD-14878 | 66.1 | 3.8 |
| XD-14880 | 64.5 | 4.5 |
| XD-14882 | 52.7 | 2.8 |
| XD-14886 | 67.8 | 4.4 |
| XD-14887 | 63.1 | 2.3 |
| XD-14889 | 51.5 | 3.5 |
| XD-14890 | 52.3 | 0.6 |
| XD-14891 | 67.7 | 5.1 |
| XD-14893 | 51.7 | 2.4 |
| XD-14900 | 66.2 | 2.8 |
| XD-14901 | 55.3 | 1 |
| XD-14902 | 72.5 | 2.1 |
| XD-14903 | 69.5 | 2.5 |
| XD-14904 | 62.2 | 2.2 |
| XD-14905 | 63.9 | 3.9 |
| XD-14906 | 76.6 | 4.8 |
| XD-14907 | 77.2 | 8.1 |
| XD-14908 | 76.5 | 6.7 |
| XD-14909 | 75.1 | 7.7 |
| XD-14917 | 59.8 | 7.9 |
| XD-14924 | 68 | 5.5 |
| XD-14949 | 75.9 | 7.4 |
| XD-14952 | 75.8 | 5.2 |

Overall, the siRNA treatment data shows successful ATXN2 mRNA knockdown.

Confirmation of ATXN2 Protein Level Reduction by siRNA Treatment

To assess whether ATXN2 protein levels were also reduced by the informatically predicted siRNAs, 56 siRNAs were resynthesized (44 top ranked siRNAs by knockdown at 200 pM; 2 additional siRNAs near the top ranked, but having ATXN2L cross-reactivity (XD-14776) or mouse cross-reactivity (XD-14887) as characteristics which merited their re-testing; additional 10 siRNAs selected by a joint assessment of the ranking by knockdown at 20 nM dosed siRNA (from the top 55 ranked by knockdown), and also taking into account an informatic prediction of off-target likelihood. These siRNAs were synthesized to a reported purity of 80-85% (Dharmacon). As before, siRNAs were synthesized as 22 nucleotide guide and passenger strands, with a 20 nucleotide complementary sequence between guide base 1-20 and passenger bases 1-20, with 2 nucleotide 3′ overhangs on each strand, and introduced by transient transfection. Three additional controls were included. A non-targeting control (NTC) (Dharmacon, ON-Target plus Control Non-Targeting siRNA #1, D-001810-01-05) and a sequence targeting luciferase controlled for any nonspecific effects of siRNA treatment, including transfection reagents, on ATXN2 signal. For the luciferase control, sense sequence: GGAATTATAATGCTTATCTATA (SEQ ID NO:536); antisense sequence: TAGATAAGCATTATAATTCCTA (SEQ ID NO:537). A ‘SMARTPool’ (SMP), a combination of 4 siRNAs targeting ATXN2 (Dharmacon; ON-TARGETplus Human ATXN2 siRNA SMARTPool, L-011772-00-0005) was used as a positive control for specific targeting of ATXN2. Both the NTC and SMARTPool siRNAs are chemically modified to limit off-target effects.

An imaging based assay used indirect immunofluorescence signal by antibodies against ATXN2 to quantify ATXN2 levels. For these experiments U2OS cells were selected because of their large and uniform cell bodies, which permit good visualization of Ataxin-2 levels in the cytoplasm. siRNAs were introduced by transient transfection, and then 3 days later cells were fixed in paraformaldehyde, and then blocked and immunostained for Ataxin-2 and counterstained with Hoechst dye 33342 to identify cell nuclei.

Images were segmented using custom pipelines developed in Cell Profiler. First, cell nuclei are identified and outlined based on Hoechst 33342 signal. Subsequently, the nuclei outline is expanded to generate a ring. Within this ring, for each cell, the signal from the indirect immunofluorescence channel corresponding to a fluorescent secondary antibody binding to anti-Ataxin-2 is quantified. To calculate the ATXN2 signal for a well, the mean across cells in the well (typically 1000-3500 cells imaged/well) of cellular ATXN2 signal was calculated. The upper quartile ATXN2 signal within the cytoplasmic region was used. By taking the upper quartile of signal, this avoids the influence of signal from segmented regions of the image that may inadvertently not contain cells.

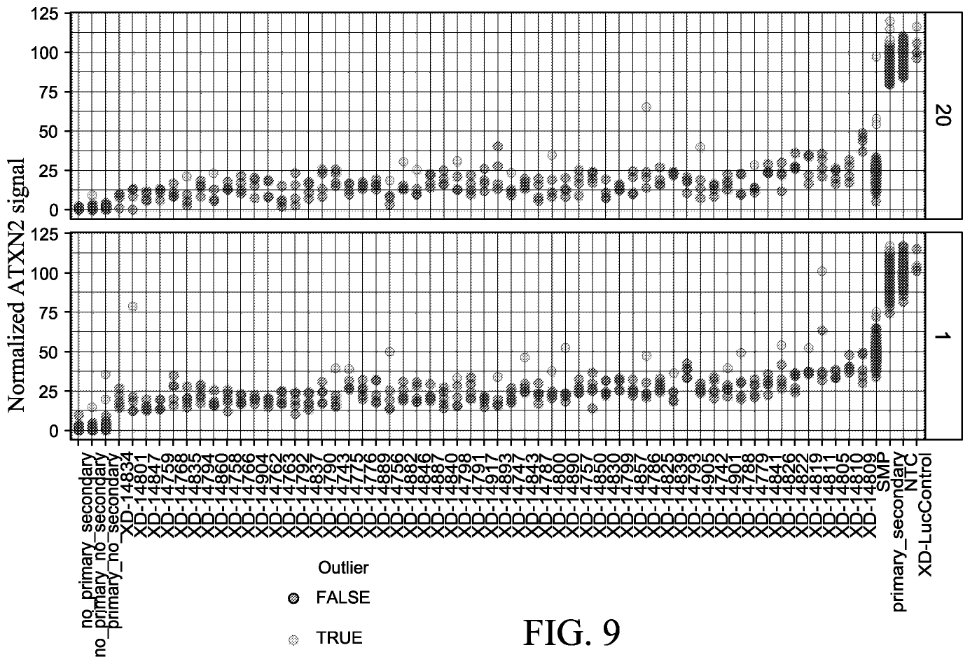

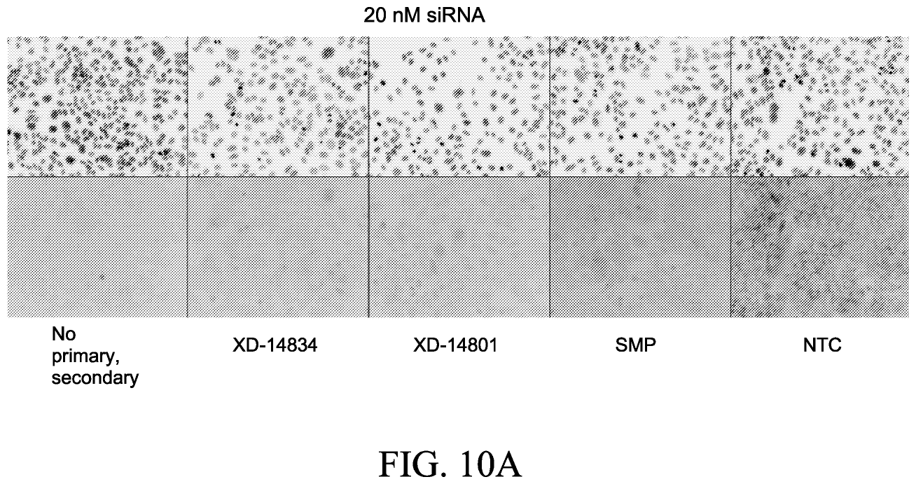

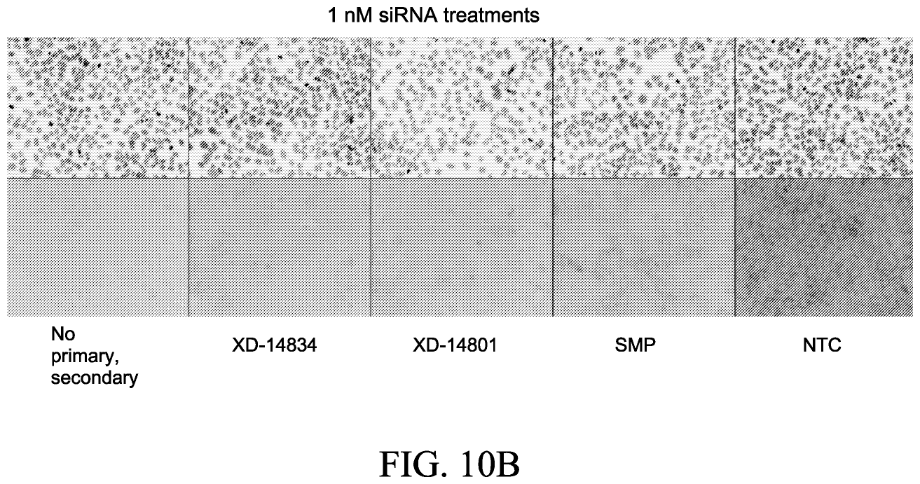

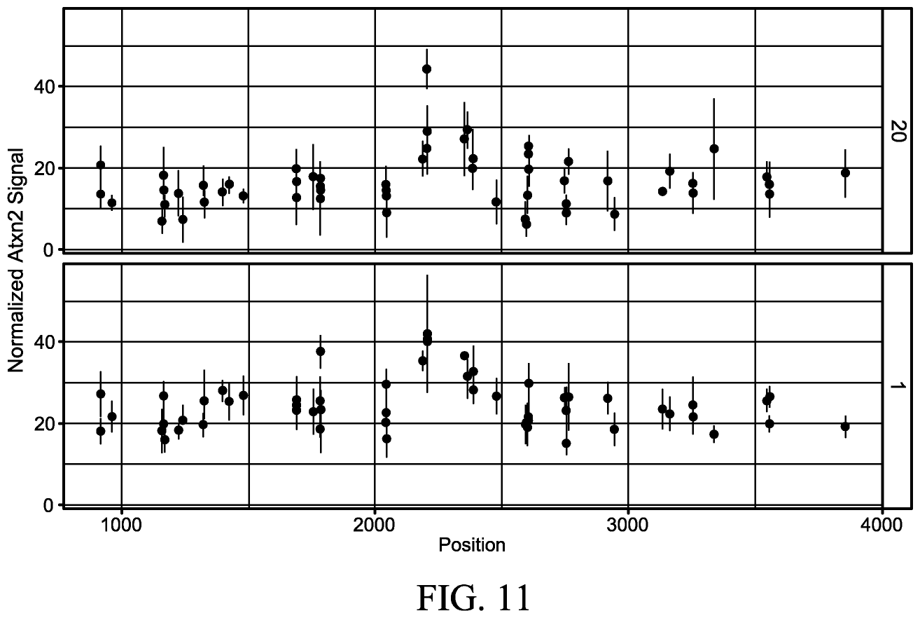

Cells were dosed with 20 or 1 nM siRNA in 96-well format, across multiple plates with controls in each plate. Background was subtracted by, within each imaging plate, wells stained with secondary antibody but not primary antibody, and not transfected. This reflects background intensity due to nonspecific binding of the secondary antibody. Ataxin-2 intensity values were normalized to those from wells transfected with non-targeting control (‘NTC’). From this, normalized ATXN2 signal represents a proxy for degree of protein level knockdown. Importantly, ATXN2 signal was similar for wells treated with luciferase targeting siRNA as with cells treated with NTC control. Note that the ‘NTC’ control (Dharmacon) chemistry is modified to reduce off-target effects whereas all ATXN2-targeting and luciferase-targeting siRNAs tested were unmodified. FIG. 9 quantifies knockdown of ATXN2 signal for siRNAs at 20 and 1 nM dose levels. FIGS. 10A and 10B show representative images from the knockdown experiments, with evidence of clear reduction of Ataxin-2 intensity from the indicated siRNAs. FIG. 11 plots the siRNAs protein knockdown data, at either 20 or 1 nM siRNA, as a function of ATXN2 transcript position. Almost all of these top siRNAs yielded substantial knockdown of siRNA at the protein level. At 1 nM, all of these top siRNAs exceeded the knockdown performance of the SMARTPool siRNA. Tables 6 and 7 display the mean and standard deviation of ATXN2 signal across wells. Sequences of the siRNAs from Tables 6 and 7 are provided in Table 1. For mean and SD calculations, outliers were excluded (outliers defined as wells where value deviated from the median value across wells by more than 1.5 standard deviations and by greater than 1000 normalized ATXN2 signal). Outlier wells are highlighted in FIG. 9.

TABLE 6
ATXN2 protein knockdown, measured by high content
imaging, after SiRNA treatment at 20 nM

| | | Mean | Standard | |
|---|---|---|---|---|
| Treatment/ | Dose | ATXN2 | Deviation | |
| Immunostain | in nM | Signal | ATXN2 Signal | N |
| no_primary_no_ | 20 | −0.7 | 2.2 | 23 |
| secondary | | | | |
| no_primary_ | 20 | 0 | 1.3 | 24 |
| secondary | | | | |
| NTC | 20 | 100 | 6.1 | 64 |
| primary_no_ | 20 | 1.7 | 1.9 | 24 |
| secondary | | | | |
| primary_secondary | 20 | 92.5 | 7.2 | 46 |
| SMP | 20 | 23.4 | 5.8 | 61 |
| XD-14742 | 20 | 13.6 | 3.5 | 4 |
| XD-14743 | 20 | 20.7 | 4.9 | 4 |
| XD-14747 | 20 | 11.4 | 1.9 | 3 |
| XD-14756 | 20 | 6.9 | 3 | 3 |
| XD-14757 | 20 | 18.2 | 6.9 | 4 |
| XD-14758 | 20 | 14.6 | 2.3 | 4 |
| XD-14759 | 20 | 11.1 | 3.2 | 4 |
| XD-14762 | 20 | 13.7 | 5.9 | 4 |
| XD-14763 | 20 | 7.4 | 5.7 | 4 |
| XD-14766 | 20 | 15.7 | 5 | 4 |
| XD-14768 | 20 | 11.6 | 3.9 | 4 |
| XD-14775 | 20 | 14.1 | 3.3 | 4 |
| XD-14776 | 20 | 15.9 | 1.7 | 4 |
| XD-14779 | 20 | 13.2 | 1.5 | 3 |
| XD-14786 | 20 | 19.8 | 4.9 | 3 |
| XD-14787 | 20 | 12.7 | 6.7 | 4 |
| XD-14788 | 20 | 16.7 | 7.4 | 4 |
| XD-14790 | 20 | 17.9 | 8.1 | 4 |
| XD-14791 | 20 | 15.6 | 5.4 | 4 |
| XD-14792 | 20 | 12.5 | 9.1 | 4 |
| XD-14793 | 20 | 17.4 | 4.4 | 4 |
| XD-14794 | 20 | 14.5 | 4.2 | 4 |
| XD-14798 | 20 | 15.9 | 4.7 | 3 |
| XD-14799 | 20 | 14.5 | 1.8 | 4 |
| XD-14800 | 20 | 13.1 | 5.7 | 3 |
| XD-14801 | 20 | 9 | 6.1 | 4 |
| XD-14805 | 20 | 22.2 | 4.3 | 4 |
| XD-14809 | 20 | 44.2 | 4.9 | 4 |
| XD-14810 | 20 | 24.8 | 6.4 | 4 |
| XD-14811 | 20 | 29 | 6.3 | 4 |
| XD-14819 | 20 | 27.1 | 9.1 | 4 |
| XD-14822 | 20 | 29.4 | 4.6 | 4 |
| XD-14825 | 20 | 19.9 | 5.2 | 4 |
| XD-14826 | 20 | 22.2 | 7.5 | 4 |
| XD-14830 | 20 | 11.7 | 5.4 | 4 |
| XD-14834 | 20 | 7.5 | 4.3 | 4 |
| XD-14835 | 20 | 6.2 | 3.5 | 3 |
| XD-14837 | 20 | 13.3 | 4.8 | 4 |
| XD-14839 | 20 | 23.4 | 0.9 | 4 |
| XD-14840 | 20 | 19.7 | 4.2 | 4 |
| XD-14841 | 20 | 25.3 | 2.7 | 4 |
| XD-14843 | 20 | 16.8 | 2.8 | 4 |
| XD-14846 | 20 | 11.2 | 2 | 3 |
| XD-14847 | 20 | 8.9 | 2.8 | 4 |
| XD-14850 | 20 | 21.6 | 3.1 | 4 |
| XD-14857 | 20 | 16.8 | 7.4 | 4 |
| XD-14860 | 20 | 8.7 | 4 | 3 |
| XD-14882 | 20 | 14.2 | 1.3 | 3 |
| XD-14887 | 20 | 19.2 | 4.2 | 4 |
| XD-14889 | 20 | 16.2 | 2.6 | 4 |
| XD-14890 | 20 | 13.8 | 4.9 | 4 |
| XD-14893 | 20 | 24.7 | 12.4 | 4 |
| XD-14901 | 20 | 17.7 | 4 | 4 |
| XD-14904 | 20 | 15.9 | 5.8 | 4 |
| XD-14905 | 20 | 13.6 | 5.7 | 3 |
| XD-14917 | 20 | 18.7 | 5.9 | 4 |
| XD-LucControl | 20 | 101 | 4.8 | 3 |

TABLE 7
ATXN2 protein knockdown, measured by high content imaging, after
siRNA treatment at 1 nM

| Treatment/ | Dose | Mean ATXN2 | Standard Deviation | |
|---|---|---|---|---|
| Immunostain | in nM | Signal | ATXN2 Signal | N |
| no_primary_no_ | 1 | −1.3 | 2.8 | 29 |
| secondary | | | | |
| no_primary_secondary | 1 | 0 | 2.7 | 30 |
| NTC | 1 | 99 | 17.1 | 78 |
| primary_no_secondary | 1 | 1.4 | 2.8 | 28 |
| primary_secondary | 1 | 97.9 | 10 | 62 |
| SMP | 1 | 50.1 | 7.1 | 78 |
| XD-14742 | 1 | 27.2 | 5.5 | 5 |
| XD-14743 | 1 | 18.1 | 3.3 | 4 |
| XD-14747 | 1 | 21.7 | 3.8 | 5 |
| XD-14756 | 1 | 18.2 | 5.5 | 4 |
| XD-14757 | 1 | 26.7 | 3.6 | 5 |
| XD-14758 | 1 | 19.8 | 5.2 | 5 |
| XD-14759 | 1 | 16 | 3.2 | 5 |
| XD-14762 | 1 | 18.3 | 2.3 | 5 |
| XD-14763 | 1 | 20.7 | 3.8 | 5 |
| XD-14766 | 1 | 19.7 | 2.9 | 5 |
| XD-14768 | 1 | 25.5 | 7.6 | 5 |
| XD-14775 | 1 | 28 | 2.5 | 4 |
| XD-14776 | 1 | 25.4 | 4.7 | 5 |
| XD-14779 | 1 | 26.8 | 4.8 | 5 |
| XD-14786 | 1 | 24.4 | 4 | 4 |
| XD-14787 | 1 | 23.2 | 4.6 | 5 |
| XD-14788 | 1 | 25.7 | 5.7 | 4 |
| XD-14790 | 1 | 22.9 | 5.6 | 5 |
| XD-14791 | 1 | 25.5 | 5.9 | 5 |
| XD-14792 | 1 | 18.6 | 5.7 | 5 |
| XD-14793 | 1 | 37.6 | 4 | 5 |
| XD-14794 | 1 | 23.4 | 4.7 | 5 |
| XD-14798 | 1 | 20.2 | 6.1 | 4 |
| XD-14799 | 1 | 29.5 | 3.7 | 5 |
| XD-14800 | 1 | 22.6 | 1.8 | 4 |
| XD-14801 | 1 | 16.2 | 4.5 | 4 |
| XD-14805 | 1 | 35.4 | 2.5 | 5 |
| XD-14809 | 1 | 40 | 8.5 | 5 |
| XD-14810 | 1 | 40.7 | 4.3 | 5 |
| XD-14811 | 1 | 42 | 14.5 | 4 |
| XD-14819 | 1 | 36.6 | 0.7 | 4 |
| XD-14822 | 1 | 31.5 | 5.6 | 5 |
| XD-14825 | 1 | 28.2 | 3.4 | 5 |
| XD-14826 | 1 | 32.7 | 6.5 | 4 |
| XD-14830 | 1 | 26.6 | 4.6 | 5 |
| XD-14834 | 1 | 19.7 | 4.9 | 5 |
| XD-14835 | 1 | 20.3 | 4.9 | 5 |
| XD-14837 | 1 | 19 | 4.6 | 5 |
| XD-14839 | 1 | 21.6 | 3.1 | 4 |
| XD-14840 | 1 | 20.7 | 5.4 | 5 |
| XD-14841 | 1 | 29.8 | 4.9 | 5 |
| XD-14843 | 1 | 26.3 | 2.5 | 4 |
| XD-14846 | 1 | 23.1 | 5.8 | 5 |
| XD-14847 | 1 | 15.1 | 2.9 | 5 |
| XD-14850 | 1 | 26.4 | 8.2 | 5 |
| XD-14857 | 1 | 26.1 | 3.8 | 5 |
| XD-14860 | 1 | 18.5 | 4 | 5 |
| XD-14882 | 1 | 23.5 | 4.9 | 5 |
| XD-14887 | 1 | 22.3 | 4.2 | 5 |
| XD-14889 | 1 | 24.4 | 7 | 5 |
| XD-14890 | 1 | 21.6 | 2.4 | 4 |
| XD-14893 | 1 | 17.3 | 2 | 3 |
| XD-14901 | 1 | 25.6 | 2.9 | 4 |
| XD-14904 | 1 | 19.9 | 2.1 | 5 |
| XD-14905 | 1 | 26.5 | 2.5 | 5 |
| XD-14917 | 1 | 19.2 | 2.7 | 5 |
| XD-LucControl | 1 | 114.6 | 12.1 | 5 |

Remarkably, 53 out of 56 of the ATXN2-targeting sequences achieved greater than 60% ATXN2 signal knockdown by this assay. In this assay, nonspecific antibody signal was not corrected. In subsequent assays (see below), ATXN2 knockout cells were used as controls demonstrating that some ATXN2 antibody background is present. Therefore, the ATXN2 protein level knockdown values here may underestimate the amount of protein knockdown caused by the ATXN2-targeting siRNA treatments.

Selection of Top-Ranked Sequences for Evaluation in siRNA Dose Response and in miRNA Backbones

To assess the potency of guide sequences targeting ATXN2, dose-response profiling of siRNAs and testing of guide sequences in miRNA format of 22 top sequences was conducted. To select top sequences for this detailed profiling, rankings of RNA knockdown for siRNAs at 20 nM and 200 pM were first assessed. In addition to this ranking of RNA knockdown, a method for predicting the number of off-target transcripts that would be influenced by the guide sequence was used, generating a probability of off-targeting score (POTS). https://sispotr.icts.uiowa.edu/sispotr/tools/lookup/evaluate.html) (Boudreau et al., Nucleic Acids Research 2013 41(1):e9). This score considers the seed sequence of the siRNA, and as such is supplementary to the initial assessment of off-target prediction based on the number of transcripts with 2 or fewer mismatches to the first 19 nucleotides of the guide sequence. Going down the knockdown ranks of siRNAs, sequences with increasingly stringent POTS score were favored. Additional criteria evaluated were: proximity to the region of ATXN2 complementarity for other guide sequences; re-examination of the number of transcripts closely complementary to nucleotides 2-19 were taken into account and resulted in the exclusion of two other sequences. The specific predicted off-targets were not examined for the selection of sequences for these experiments.

In addition to top-ranked sequences, two low-performing siRNAs (XD-14781 and XD-14949) that had low mRNA knockdown when assessed as siRNAs at 20 nM or 1 nM, were included to confirm the range and sensitivity of downstream assays.

siRNA Dose Response Versus ATXN2 mRNA Knockdown Testing

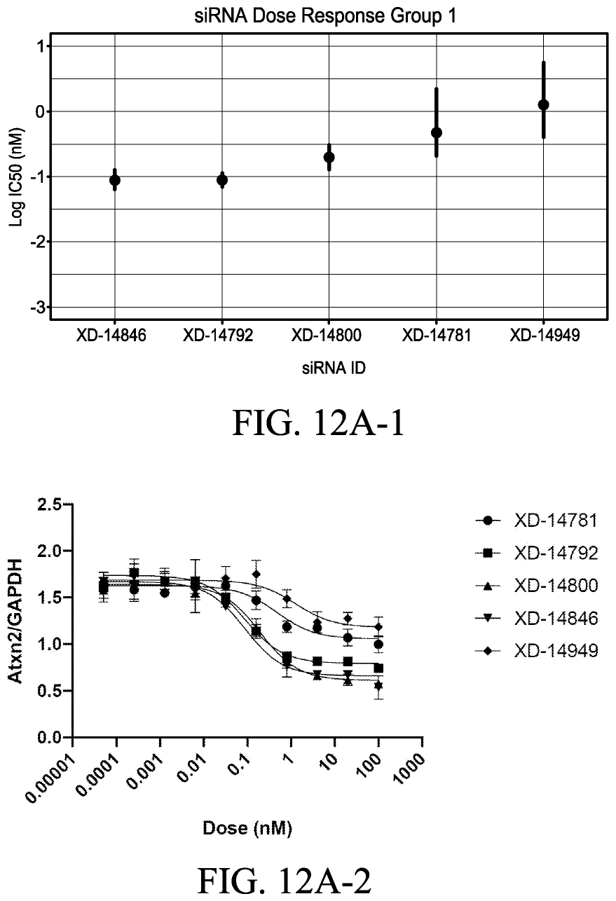

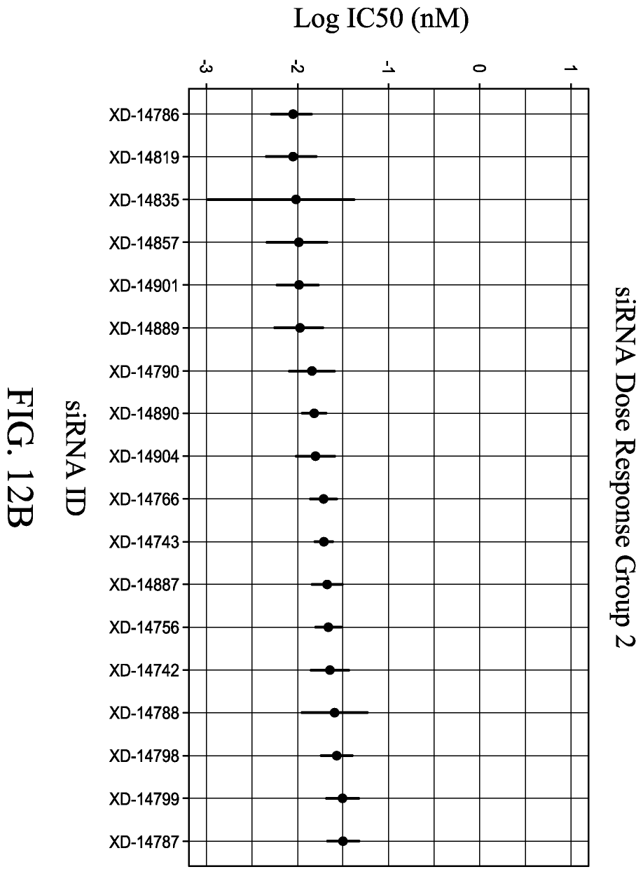

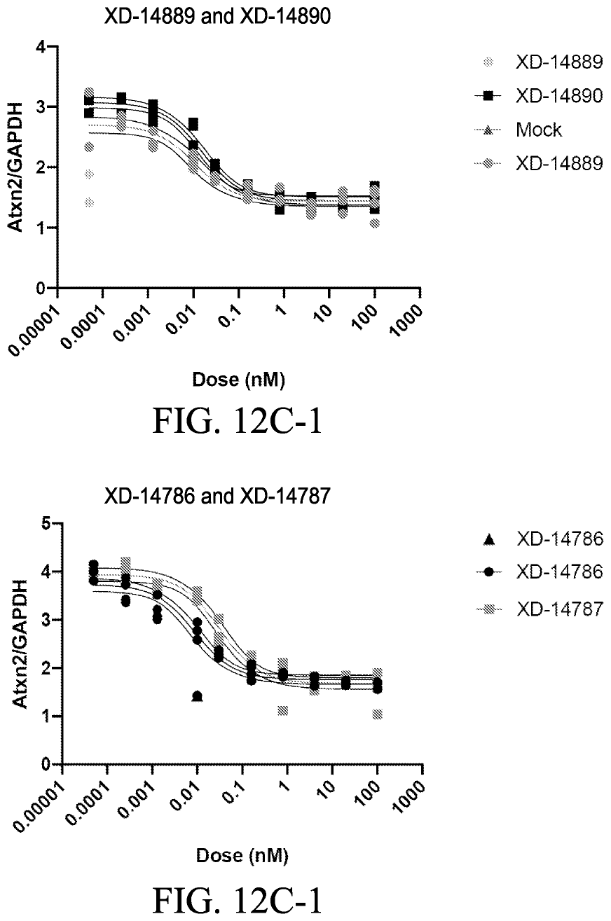

Dose response profiling was performed by testing dilution series of siRNAs transfected into HepG2 cells (FIG. 12), as described above for single-dose experiments. As expected, sequences XD-14781 (guide SEQ ID NO:90; passenger SEQ ID NO:89) and XD-14949 (guide SEQ ID NO:426; passenger SEQ ID NO:425), which had poor performance when assessed at 20 and 1 nM, had low potency and reduced maximal knockdown when assessed in dose response. IC50s of all other top-ranked siRNAs separated from these values. Two batches of testing were performed. Top sequences from one of the batches were estimated to have concentrations achieving half-maximal knockdown of <10 pM, indicating that the top-ranked siRNAs are highly potent. Performances of siRNAs had some dissimilarities between the batches but this was not investigated further, and the sequences were advanced into further testing in miRNA format. This miRNA testing, discussed below, showed that the lowest performing sequences from each batch were separated from the highest performing sequences in efficacy of ATXN2 protein lowering, but that the performance of top siRNAs from the two batches were similar. The miRNA testing is therefore regarded as more relevant for precise ranking of sequences.

Design and Production of ATXN2-Targeting Sequences in miRNA Backbones

Following identification of active siRNA sequences, siRNAs were embedded in miRNAs for expression from DNA vectors. The miR-155 and miR-1-1 backbones were considered.

The miR-155 was originally identified as a promising scaffold for construction of RNA polymerase II-based miRNA vectors due to its location within a conserved non-coding RNA⁸. After initial identification and design of miR-155 shRNA, subsequent sequence improvements increased microprocessor cleavage³. Many groups took the miR-155 scaffold to preclinical use in mice^{10,11}, sheep¹² and non-human primates¹³, enabling gene therapy approaches in genetically-driven human disease.

Initial experiments were conducted using a version of the miR-155 scaffold that, in one previous report, was engineered into an artificial mRNA and used in a mouse in vivo proof of concept study to knockdown HTT¹⁰. Small RNA sequencing had demonstrated high strand bias by this miRNA backbone¹⁰. ATXN2 targeting guide sequences and controls were incorporated into this scaffold sequence, which was termed “mR-155M” and assayed for protein knockdown after transfection of U2OS cells.

To rationally improve miR-155, human genomic sequence was examined, and the span of flanking miR-155 sequence to be used was defined by the region surrounding miR-155 with high evolutionary conservation across similar species. That is, a plot of sequence conservation versus position was visualized, and the genomic position from the endogenous miR-155 at which this sequence conservation dropped off was used to determine how much flanking context around the miR-155 stem structure should be included. Next, the mIR-155 loop was examined for features which might impact the use of this miR in different expression systems. A homotetrameric UUUU in the miR-155 loop was noted. UUUU sequences have been reported to induce Polymerase III termination¹⁴, which would lead to aberrantly truncated miRNAs which do not undergo stem pairing. To interrupt this homotetrameric UUUU, an apical UGU motif within the miR-155 loop was added. This motif additionally has been reported to enhance miRNA processing.^{1,2} In addition to previously engineered UG and CNNC motifs³, a basal stem mismatched GHG motif² was added to improve precise processing.

To expand the number of amiRNA scaffolds beyond the miR-155 backbones, backbones from endogenous miRNAs reported to have high processing precision were prioritized. The miR-1-1 backbone ranks among the highest in processing precision according to reference:¹⁵, has high strand bias by small RNAseq⁵, and the guide strand is on the 3 prime arm of the miRNA stem, which may improve processing accuracy compared to 5 prime-arm positioned guide strands¹⁶. Natively integrated favorable sequence motifs include a basal mismatched GHG motif and downstream CNNC motif. It also has a short context for sequencing and has been successfully engineered for artificial miRNA expression in Drosophila models¹⁷.

Additional miRNA scaffolds that may be considered for the amiRNAs of the present disclosure include:
miR-100 and miR-190a—high throughput screen identified high on-target/off-target ratio¹⁵.
miR-124 and miR-132—both motor-neuron expressed miRNAs do not change expression in an ALS rat model¹⁸. The cell-type specific expression and consistent levels throughout ALS disease course are favorable miRNA characteristics. Neuronal specificity has been confirmed in a sRNAseq cross-tissue expression database¹⁹ (https://ccb-web.cs.uni-saarland.de/tissueatlas/).
miR-9—neuron-specific expression²⁰.
miR-138-2, miR-122, miR-130a, and miR-128 were selected to be naturally asymmetric (either exclusively 5′ or 3′ strand is observed in small RNAseq datasets), highly homogeneous (i.e. high “5′ homogeneity score”¹⁵), not reported to undergo post-transcriptional regulation (e.g. which occurs for clustered miRNAs), are consensus miRNAs on miRBase, have flexible loop structure and simple duplex stem.

To further mimic the miRNA backbones, bulges and mismatches can be inserted into the guide:passenger strand duplex in a manner to replicate the bulge pattern observed in endogenous miRNAs, but applied to artificial miRNAs targeting ATXN2. The modifications that can be done to the passenger strand to introduce these native-miRNA mimicking structures are provided in Table 8.

TABLE 8
Design Rules for Exemplary miRNA formats

| miR | Modifications to passenger sequence |
|---|---|
| miR-1-1 | Mismatch base 2 \| Bulge mismatch transversion base 19 \| |
| | Insert a base between bases 18 and 19, either G or C but |
| | not complementary to the guide |
| miR155 | Delete bases 9 and 13 of passenger |
| (M or E) | |
| miR100 | Bulge mismatch transversion base 11 \| Bulge mismatch |
| | transition bases 7, 18 \| add GU wobble base 17 \| Mismatch |
| | base 22 |
| miR124 | Mismatch base 3 \| Bulge mismatch transition bases 2, 17 \| |
| | Bulge mismatch transversion 12, 16 |
| miR138-2 | Mismatch bases 5, 21 \| Bulge mismatch bases 6 \| Add GU |
| | wobble base 13 \| delete bases 7, 22 |
| miR122 | Bulge mismatch transition base 12 \| Bulge mismatch |
| | transversion bases 20, 21 |
| miR-128 | Truncate guide to 21 bp \| Mismatch bases 1, 2, 12 \| Bulge |
| | mismatch transversion bases 3, 11, 13 \| Insert 2 bases |
| | between bases 20 and 21, not complementary to the guide \| |
| | add GU wobbles bases 5, 7 |
| miR130a | Mismatch base 2 \| Add GU wobbles bases 18, 19 \| bulge |
| | mismatch transversion base 11 \| Bulge mismatch transition |
| | base 22 |
| miR 16-2 | Bulge mismatch transversion bases 11, 12 \| Mismatch base |
| | 22 |

Note:
For the above, ‘passenger’ sequence refers to a sequence complementary to the 22 nucleotides of the guide sequence. This is not the same as passenger sequences as used in describing siRNA duplexes. Mismatch refers to the following substitution rule: G → C, C → G, A → T, T → A. Bule mismatch transition refers to the rule: T → C, C → A, A → C, G → A. Bulge mismatch transversion refers to the rule: G → T, C → A, A → C, T → G. Add GU wobble refers to the rule: If base is C, then convert to T.

Initial Testing of ATXN2 Targeting Guide Sequences in miR155-M and miR1-1 Backbones

As an initial test of the ability of the Atxn2 targeting siRNAs to knock down Atxn2 when embedded in a miRNA context, the guide sequence of XD-14792 (SEQ ID NO:112), which had the highest ranked ATXN2 mRNA knockdown when dosed at 200 pM as an siRNA, was embedded in several miRNA contexts as shown in Table 9. The amiRNA DNA sequences are provided in Table 9 as SEQ ID NOS:538-543. The corresponding amiRNA RNA sequences are provided in Table 9 as SEQ ID NOS:1109-1114, respectively.

TABLE 9
XD-14792 sequences embedded in amiRNAs

| | Target/ | Guide | | miR | Artificial miRNA | Artificial miRNA |
|---|---|---|---|---|---|---|
| Name | Type | Sequence | Variation | Backbone | (DNA Sequence) | (RNA Sequence) |
| XD-14792_miR1-1 | ATXN2 | AUUAACUACUCUU | | 1-1 | CATGCAGACTGCCTGCTTGGGTA | CAUGCAGACUGCCUGCUUGGGU |
| | | UGGUCUGAA | | | CAGACCAAAGAGTAGTCGAATTA | ACAGACCAAAGAGUAGUCGAAU |
| | | [SEQ ID NO: 112] | | | TGGACCTGCTAAGCTAATTAACT | UAUGGACCUGCUAAGCUAAUUA |
| | | | | | ACTCTTTGGTCTGAACTCAGGCC | ACUACUCUUUGGUCUGAACUCA |
| | | | | | GGGACCTCTCTCGCCGCACTGAG | GGCCGGGACCUCUCUCGCCGCAC |
| | | | | | GGGCACTCCACACCACGGGGGCC | UGAGGGGCACUCCACACCACGG |
| | | | | | [SEQ ID NO: 538] | GGGCC |
| | | | | | | [SEQ ID NO: 1109] |
| XD-14792_miR1-1 | ATXN2 | AUUAACUACUCUU | E | 1-1 | CATGCAGACTGCCTGCTTGGGTA | CAUGCAGACUGCCUGCUUGGGU |
| enhanced | | UGGUCUGAA | | | CAGACCAAAGAGTAGTCGAATTA | ACAGACCAAAGAGUAGUCGAAU |
| | | [SEQ ID NO: 112] | | | TGGACCTGCTAAGCTAATTAACT | UAUGGACCUGCUAAGCUAAUUA |
| | | | | | ACTCTTTGGTCTGAACTCAGGCC | ACUACUCUUUGGUCUGAACUCA |
| | | | | | GGGACCTCTTCCGCCGCACTGAG | GGCCGGGACCUCUUCCGCCGCAC |
| | | | | | GGGCACTCCACACCACGGGGGCC | UGAGGGGCACUCCACACCACGG |
| | | | | | [SEQ ID NO: 539] | GGGCC |
| | | | | | | [SEQ ID NO: 1110] |
| XD-14792_miR155 | ATXN2 | AUUAACUACUCUU | | 155 | CCTGGAGGCTTGCTGAAGGCTGT | CCUGGAGGCUUGCUGAAGGCUG |
| | | UGGUCUGAA | | | ATGCTGATTAACTACTCTTTGGTC | UAUGCUGAUUAACUACUCUUUG |
| | | [SEQ ID NO: 112] | | | TGAATTTTGGCCACTGACTGATTC | GUCUGAAUUUUGGCCACUGACU |
| | | | | | AGACCAAGGTAGTTAATCAGGAC | GAUUCAGACCAAGGUAGUUAAU |
| | | | | | ACAAGGCCTGTTACTAGCACTCA | CAGGACACAAGGCCUGUUACUA |
| | | | | | CATGGAACAAATGGCCACCGG | GCACUCACAUGGAACAAAUGGC |
| | | | | | [SEQ ID NO: 540] | CACCGG |
| | | | | | | [SEQ ID NO: 1111] |
| XD14792_911_miR155 | ATXN2 | AUUAACUAGAGUU | | 155 | CCTGGAGGCTTGCTGAAGGCTGT | CCUGGAGGCUUGCUGAAGGCUG |
| | 911 | UGGUCUGAA | | | ATGCTGATTAACTAGAGTTTGGT | UAUGCUGAUUAACUAGAGUUUG |
| | Control | [SEQ ID NO: 544] | | | CTGAATTTTGGCCACTGACT | GUCUGAAUUUUGGCCACUGACU |
| | | | | | GATTCAGACCAACCTAGTTAATC | GAUUCAGACCAACCUAGUUAAU |
| | | | | | AGGACACAAGGCCTGTTACTAGC | CAGGACACAAGGCCUGUUACUA |
| | | | | | ACTCACATGGAACAAATGGC | GCACUCACAUGGAACAAAUGGC |
| | | | | | CACCGG | CACCGG |
| | | | | | [SEQ ID NO: 541] | [SEQ ID NO: 1112] |
| XD-14792_SScr | ATXN2 | AUUAACUAAGUAU | | 155 | CCTGGAGGCTTGCTGAAGGCTGT | CCUGGAGGCUUGCUGAAGGCUG |
| miR155 | SScr | CGGUCUCUU | | | ATGCTGATTAACTAAGTATCGGT | UAUGCUGAUUAACUAAGUAUCG |
| | | [SEQ ID NO: 545] | | | CTCTTTTTTGGCCACTGACTGAAA | GUCUCUUUUUUGGCCACUGACU |
| | | | | | GAGACCATATTAGTTAATCAGGA | GAAAGAGACCAUAUUAGUUAAU |
| | | | | | CACAAGGCCTGTTACTAGCACTC | CAGGACACAAGGCCUGUUACUA |
| | | | | | ACATGGAACAAATGGCCACCGG | GCACUCACAUGGAACAAAUGGC |
| | | | | | [SEQ ID NO: 542] | CACCGG |
| | | | | | | [SEQ ID NO: 1113] |
| XD-14792_miR155 | ATXN2 | AUUAACUACUCUU | S | 155 | CCTGGAGGCTTGCTGAAGGCTGT | CCUGGAGGCUUGCUGAAGGCUG |
| sealed | | UGGUCUGAA | | | ATGCTGATTAACTACTCTTTGGTC | UAUGCUGAUUAACUACUCUUUG |
| | | [SEQ ID NO: 112] | | | TGAATTTTGGCCACTGACTGATTC | GUCUGAAUUUUGGCCACUGACU |
| | | | | | AGACCAAAGAGTAGTTAATCAGG | GAUUCAGACCAAAGAGUAGUUA |
| | | | | | ACACAAGGCCTGTTACTAGCACT | AUCAGGACACAAGGCCUGUUAC |
| | | | | | CACATGGAACAAATGGCCACC | UAGCACUCACAUGGAACAAAUG |
| | | | | | [SEQ ID NO: 543] | GCCACC |
| | | | | | | [SEQ ID NO: 1114] |

In the variation column: “E” refers to “enhanced,” “S” refers to ‘sealed’

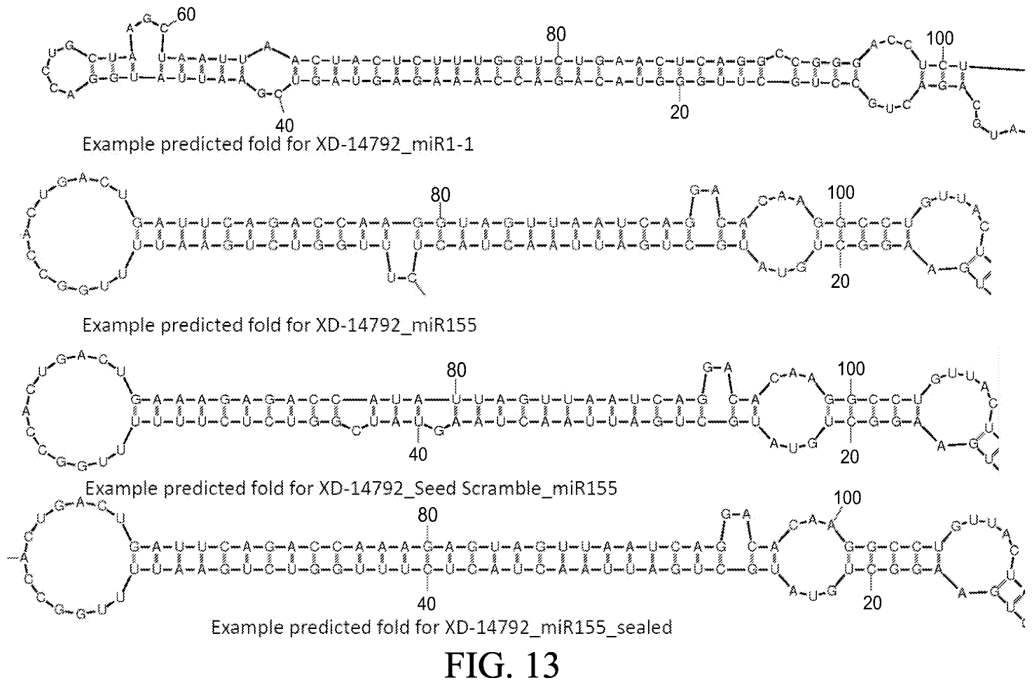

In Table 9, the guide sequences (including the guide sequence, any variants, as well as the parental guide sequence from which they are derived) are shown in RNA form, and the artificial miR sequence is provided in both RNA format, and for when embedded in the vector is shown in DNA form. The miR backbones used include: (a) miR155, preserving a bulge format reported in (Fowler et al., Nucleic Acids Res. (2015) 44:e48); (b) miR155, with no sequence bulges, yielding a perfectly complementary stem (“sealed”); (c) miR1-1, preserving a native bulge format as in the endogenous miRNA; and (d) miR1-1 with the “Enhanced” variation, including a modification in the 3′ arm that in other miRNAs was previously reported to enhance processing (Auyeung et al., Cell 2013). FIG. 13 shows one of the predicted RNA folds of the miRNA stems of several of the constructs, using the web server mfold. Bulges in the stem in the region including or apposed to the guide sequence are apparent, which are designed to mimic the native mismatches of the endogenous forms of the microRNAs from which derive the surrounding context for the guide sequence. As controls (“911 controls”), bases 9, 10, and 11 of XD-14792 guide sequence were modified to be the complementary bases (that is, substituting A->T, T->A, C->G, or G->C); or (“SScr”), in which all bases except bases 1-7 were scrambled. In both cases, any seed-mediated off-target activity (deriving from bases 1-7) should be preserved, whereas the on-target Atxn2 slicing activity should be blocked.

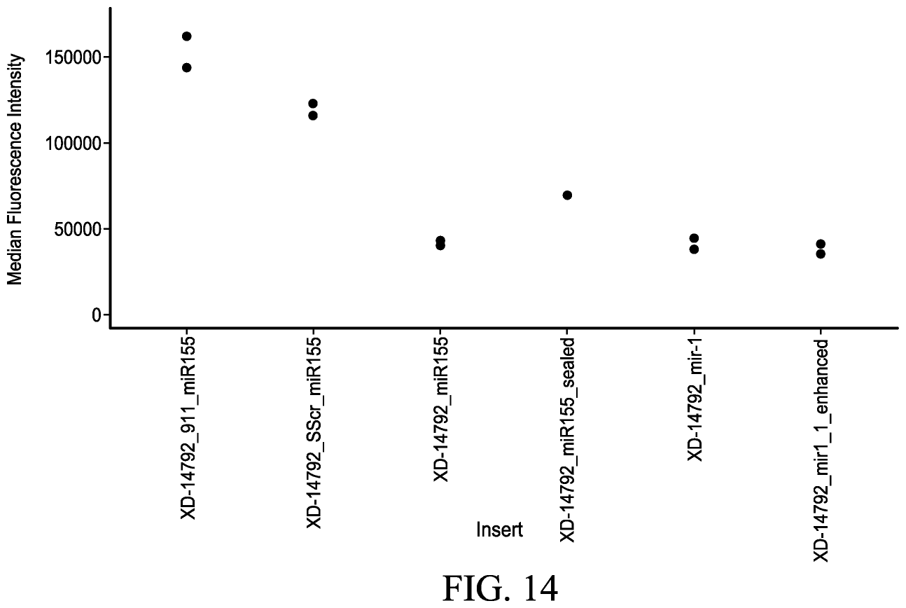

pLVX-EF1A_mCherry-miR-1-1-XD_14890-WPRE_CMV (SEQ ID NO:546) is a representative lentiviral vector that can be used for expressing these artificial microRNAs. Nucleotides 4275-4412 of SEQ ID NO:546 (XD-14890 guide sequence in a miR-1-1 backbone) can be substituted with another artificial miRNA of interest. In this lentiviral vector suitable for packaging into lentivirus, an EF1-alpha promoter drives expression of a mCherry protein. After a stop codon, the amiRNA stem is expressed downstream within a 3′ UTR. Downstream of that a WPRE element (Woodchuck Hepatitis Virus Posttranscriptional Regulatory Element) enhances the stability of the transcript. Adapters may be included upstream or downstream of the artificial miRNA construct to facilitate cloning and downstream detection of the sequences, but these adapters are not expected to influence the performance of the microRNA. A CMV promoter (as in sequence shown), or a PGK promoter (as in plasmids transfected for data shown FIG. 14), downstream, drives expression of the puromycin resistance protein for puromycin selection in mammalian cells. This is a similar design to the vector used in (Kampmann et al., PNAS 2015).

pcDNA3.1 NEGFP STOP ATXN2 3′UTR.gb (SEQ ID NO:547) represents a plasmid used to generate a GFP-ATXN2 reporter line. A CMV promoter was used to drive the expression of a transcript encoding enhanced green fluorescent protein (EGFP). A stop codon at the end of the EGFP open reading frame was followed by the ATXN2 sequence, but removing the initial ATG such that the sequence is expected to not be translated. A separate SV40 promoter downstream drives the expression of the NeoR/KanR protein product which enabled selection of U2OS cells stably integrating the plasmid by G418 selection. EGFP fluorescence was bright and diffuse, and not restricted to the cytoplasm as expected if the ATXN2 protein was translated and fused to the EGFP. Several lines were generated by single-cell cloning after G418 selection, and one line ultimately selected based on uniform fluorescent signal distribution by FACS as well as a larger differential between control-transfected (siNTC) and ATXN2 siRNA-transfected cells.

Constructs with the artificial miRNAs noted above were transfected into U2OS cells stably expressing the GFP-Atxn2 reporter by transient transfection (lipofectamine 3000). Four days later, GFP-ATXN2 levels were quantified by fluorescence automated cell sorting (FACS), gating cells by the expression of the mCherry encoded on the miRNA vector to isolate cells expressing the artificial miRNA construct. FIG. 14 shows median fluorescence intensity signal of GFP intensity. XD-14792 sequences embedded in artificial miRNA backbones miR-155 or miR-1-1 considerably reduced ATXN2 GFP reporter intensity relative to cells expressing control constructs (XD-14792 911 and XD-14792 Sscr, embedded in the miR-155 stem backbone). A ‘sealed’ XD-14792 construct in a miR-155 backbone, in which the stem is perfectly complementary (FIG. 14) did not reduce the ATXN2 GFP reporter signal as much as did XD-14792 when embedded in either miR-155 or miR-1-1 with bulged residues.

Expanded Screening of ATXN2 Targeting Sequences in Artificial microRNA Vectors in Lentiviral Format

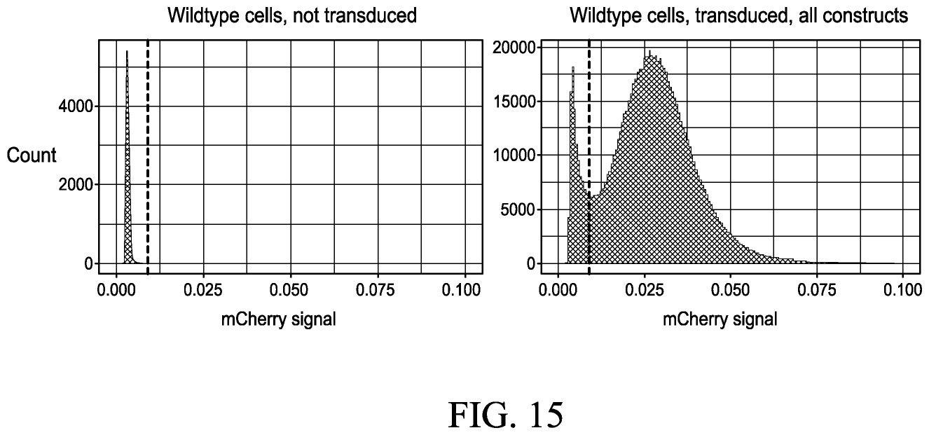

Given the encouraging results with the knockdown of the ATXN2 GFP reporter, a set of ATXN2 targeting sequences was cloned into the artificial microRNA expressing vector described above (SEQ ID NO:546). The same set of ATXN2 targeting sequences as were tested in dose-response testing for mRNA knockdown were incorporated into plasmids to enable lentiviral packaging. Vectors were packaged into lentivirus (see methods below) and transduced into unmodified U2OS cells or U2OS cells deficient for ATXN2 (described below) in a 96-well format, across multiple plates. Each plate had controls to enable plate-wise signal normalization. 3.5 days after transduction, cells were fixed with paraformaldehyde, blocked and stained with anti-ATXN2 antibodies, anti-mCherry antibodies, and Hoechst dye (33342) to demarcate cellular nuclei, and ATXN2 signal was quantified by image segmentation and signal intensity measurement as described above. Transduced and untransduced cells were differentiated by anti-mCherry signal. FIG. 15 shows histograms of the expected mCherry signal for untransduced cells as well as wildtype transduced cells. The threshold was set such that the signal from untransduced wild-type cells did not exceed this threshold, but most of the cells in the right peak of the bimodally distributed signals (right panel, wild-type transduced cells) were considered positive.

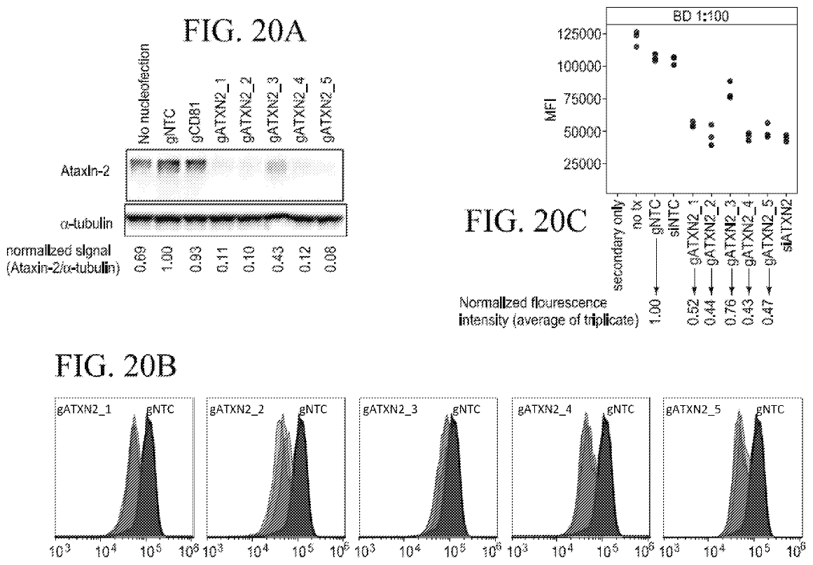

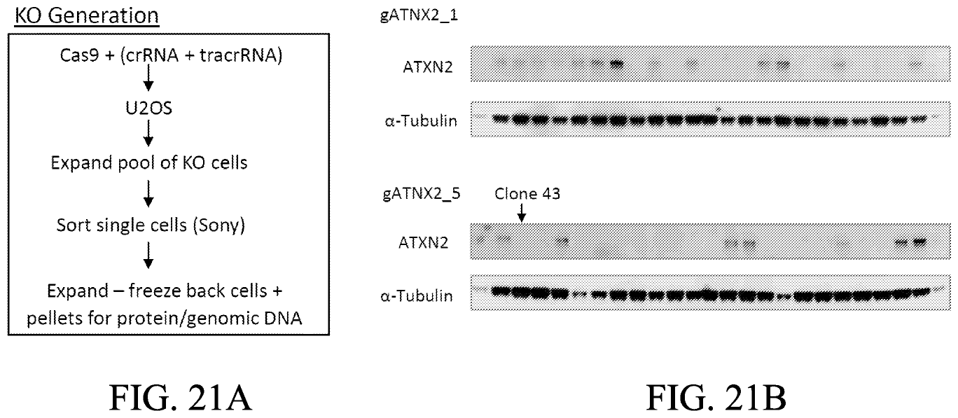

ATXN2 signal was subtracted for background measured in U2OS cells with the ATXN2 gene disrupted by CRISPR and in which ATXN2 protein had been verified to be eliminated by Western analysis. FIGS. 20-21 show the data for the knockout generation process. FIG. 20 shows Western and FACS analysis of Ataxin-2 signal in cells nucleofected with Cas9-gRNA complexes targeting Ataxin-2 or control targets. Robust reduction of Ataxin-2 protein is seen with multiple guides, consistent with editing and disruption of the Ataxin-2 gene. FIG. 21A shows the workstream to generate clonal ATXN2 knockout cells, and FIG. 21B shows Western analysis of single-cell clones derived from Cas9-gRNA nucleofected cells, from which clone 43 was confirmed to be null for Ataxin-2 and selected for further use. The clone was sequenced by Sanger sequencing, and using the ICE tool (Synthego), a mixture of disrupting mutations consistent with disruption of the ATXN2 alleles was confirmed.

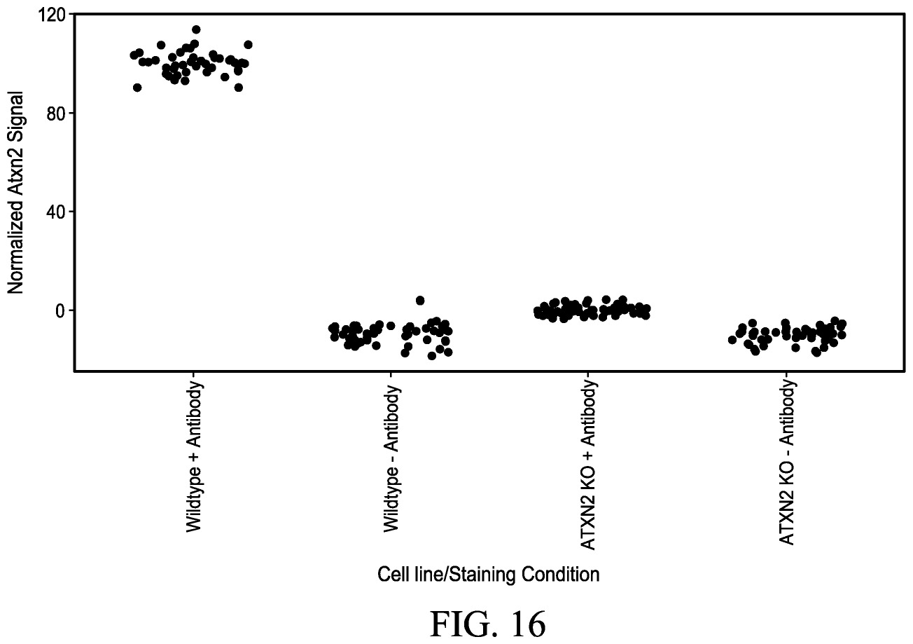

As shown in FIG. 16, the signal in ATXN2 deleted cells was slightly increased relative to wild-type cells that were treated with secondary antibody but not primary anti-Ataxin-2 antibody, suggesting some nonspecific, background binding of the ATXN2 antibody. These cells were not transduced with virus. After background subtraction, signal was normalized relative to ATXN2 signal in untransduced wild-type cells.

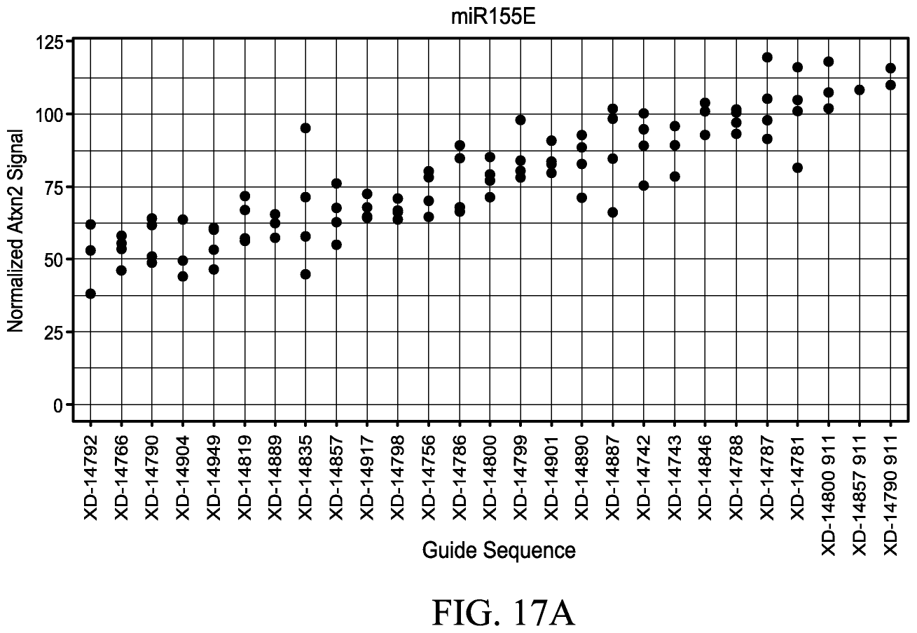

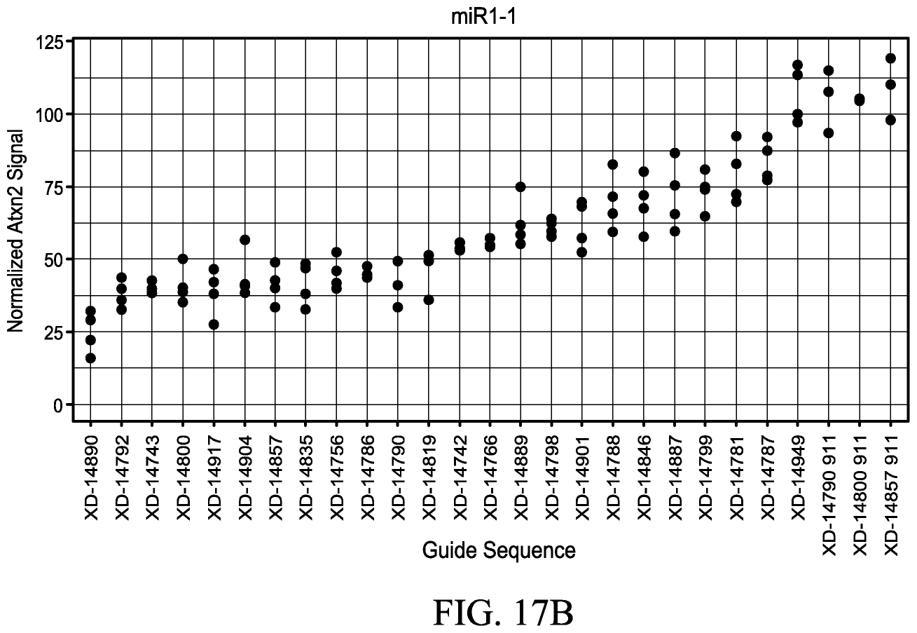

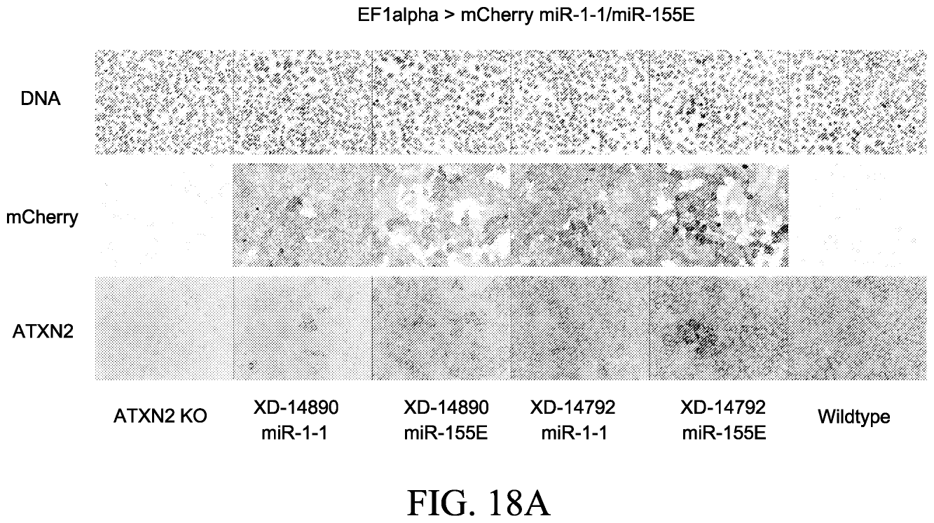

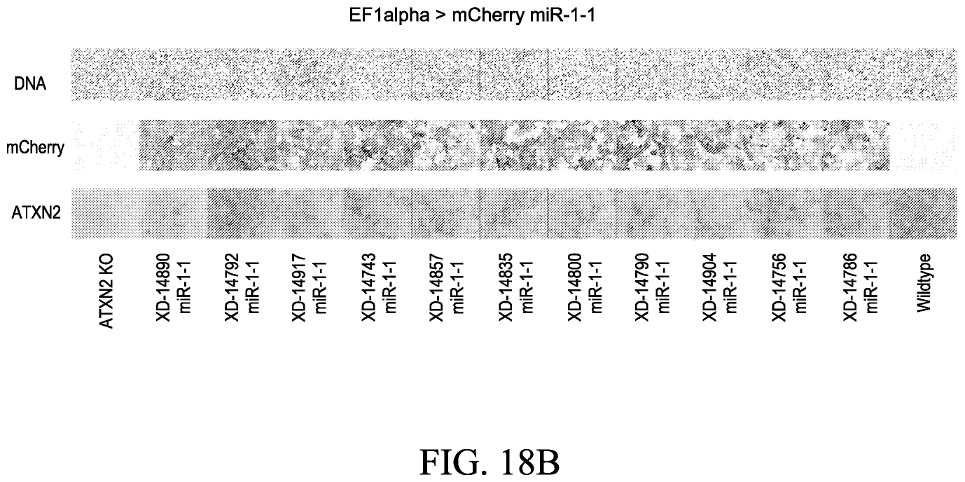

FIG. 17 shows well-level quantification of ATXN2 signal intensities across artificial microRNA constructs, with representative images shown in FIG. 18. Transduced cells were identified by anti-mCherry levels exceeding the threshold defined above. A median of 3355 cells per well were mCherry positive and included for ATXN2 signal calculation, with a range of 2469-4582 cells and standard deviation of 391 cells per well.

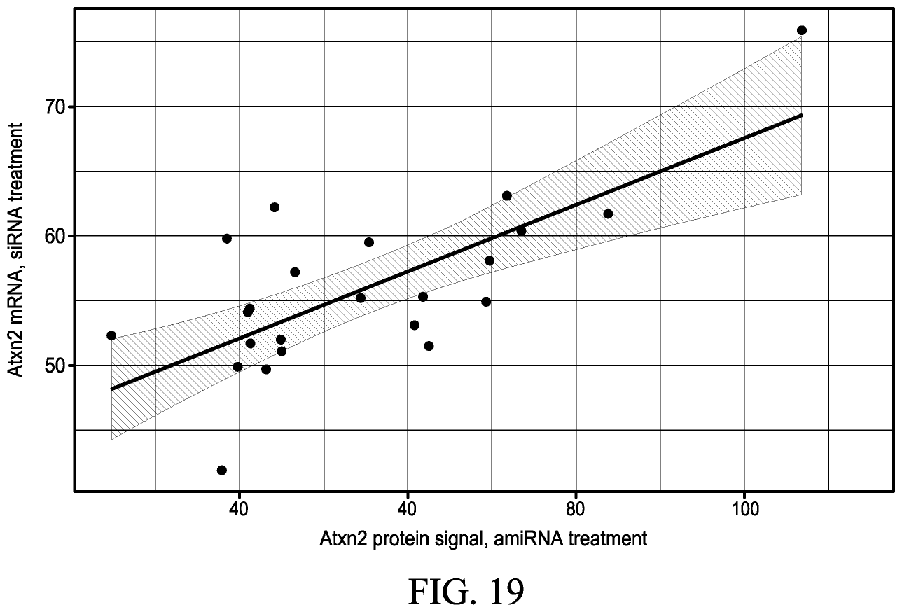

Table 10 shows mean and standard deviations of ATXN2 signals, normalized as above, for sequences, embedded either in the enhanced miR-155 backbone or the miR1-1 backbone (sequences provided in Table 11). In general, for most but not all sequences, ATXN2 knockdown performance was superior when the guide sequence was embedded in the miR1-1 backbone. None of the 911 controls, where the artificial microRNA was engineered such that guide bases 9, 10 and 11 were complemented (A->T, T->A, C->G, or G->C), exhibited knockdown, indicating that the reduction in ATXN2 signal is dependent on the direct RNA interference activity of the microRNAs on the endogenous ATXN2 transcript. Additionally, protein level knockdown across guide sequences, when examined in the miR-1-1 backbone, correlated with mRNA knockdown in HepG2 cells after 200 pM siRNA treatment. (linear model p<0.001; R²=0.5; FIG. 19).

TABLE 10
ATXN2 protein levels following amiRNA treatment

| Duplex | miR | Mean normalized | Standard |
|---|---|---|---|
| ID/Category | Backbone | ATXN2 Signal | Deviation |
| XD-14790 911 | miR-1-1 | 111.7 | 15.9 |
| XD-14790 911 | miR-155E | 116.8 | 5.4 |
| XD-14800 911 | miR-1-1 | 113.3 | 10.1 |
| XD-14800 911 | miR-155E | 111.1 | 8 |
| XD-14857 911 | miR-1-1 | 112.7 | 11.5 |
| XD-14857 911 | miR-155E | 116.7 | 10.1 1 |
| XD-14742 | miR-1-1 | 54.4 | 1.3 |
| XD-14742 | miR-155E | 89.8 | 10.7 |
| XD-14743 | miR-1-1 | 39.8 | 2 |
| XD-14743 | miR-155E | 89.9 | 8.1 |
| XD-14756 | miR-1-1 | 44.9 | 5.4 |
| XD-14756 | miR-155E | 73.2 | 7.3 |
| XD-14766 | miR-1-1 | 55.4 | 1.2 |
| XD-14766 | miR-155E | 53.3 | 5.1 |
| XD-14781 | miR-1-1 | 79.2 | 10.3 |
| XD-14781 | miR-155E | 100.8 | 14.4 |
| XD-14786 | miR-1-1 | 45 | 1.7 |
| XD-14786 | miR-155E | 77 | 11.4 |
| XD-14787 | miR-1-1 | 83.8 | 6.9 |
| XD-14787 | miR-155E | 103.4 | 12 |
| XD-14788 | miR-1-1 | 69.7 | 9.8 |
| XD-14788 | miR-155E | 98 | 3.8 |
| XD-14790 | miR-1-1 | 43.2 | 7.6 |
| XD-14790 | miR-155E | 56.3 | 7.6 |
| XD-14792 | miR-1-1 | 37.9 | 4.8 |
| XD-14792 | miR-155E | 51.5 | 9.9 |
| XD-14798 | miR-1-1 | 60.8 | 2.6 |
| XD-14798 | miR-155E | 66.8 | 3 |
| XD-14799 | miR-1-1 | 73.5 | 6.7 |
| XD-14799 | miR-155E | 84.9 | 8.9 |
| XD-14800 | miR-1-1 | 41 | 6.4 |
| XD-14800 | miR-155E | 78.1 | 5.7 |
| XD-14819 | miR-1-1 | 46.6 | 7.1 |
| XD-14819 | miR-155E | 63 | 7.5 |
| XD-14835 | miR-1-1 | 41.3 | 7.4 |
| XD-14835 | miR-155E | 67.3 | 21.5 |
| XD-14846 | miR-1-1 | 69.3 | 9.3 |
| XD-14846 | miR-155E | 97.5 | 5.6 |
| XD-14857 | miR-1-1 | 41.2 | 6.3 |
| XD-14857 | miR-155E | 65.3 | 8.9 |
| XD-14887 | miR-1-1 | 71.8 | 11.7 |
| XD-14887 | miR-155E | 87.8 | 16.3 |
| XD-14889 | miR-1-1 | 62.5 | 8.8 |
| XD-14889 | miR-155E | 62.6 | 3.7 |
| XD-14890 | miR-1-1 | 24.8 | 7.2 |
| XD-14890 | miR-155E | 83.8 | 9.2 |
| XD-14901 | miR-1-1 | 61.8 | 8.3 |
| XD-14901 | miR-155E | 84 | 4.7 |
| XD-14904 | miR-1-1 | 44.2 | 8.3 |
| XD-14904 | miR-155E | 55.2 | 9.9 |
| XD-14917 | miR-1-1 | 38.5 | 8.1 |
| XD-14917 | miR-155E | 67.2 | 3.8 |
| XD-14949 | miR-1-1 | 106.8 | 9.7 |
| XD-14949 | miR-155E | 55.2 | 6.7 |

Table 11 provides the parent guide RNA sequences, amiRNA sequences, and amiRNA DNA sequences as embedded in microRNA backbone-expressing vectors of both active guide sequences as well as a small set of control sequences. The guide sequence anticipated to be produced in cells is described in RNA form, and the sequence encoding the guide sequence (embedded in miRNA) is provided in DNA form.

TABLE 11
amiRNA Sequences

| | | miR | | | |
|---|---|---|---|---|---|
| Parent Guide | ID | Backbone | Category | amiRNA DNA Sequence | amiRNA RNA Sequence |
| AGGAACGUGGGUU | XD-14857 | miR-1-1 | 911 Control | CATGCAGACTGCCTGCTTGGGATG | CAUGCAGACUGCCUGCUUGGGAU |
| GAACUCCUU [SEQ | | | | GAGTTCAAGGGACGTCGCCTTATG | GGAGUUCAAGGGACGUCGCCUUA |
| ID NO: 242] | | | | GACCTGCTAAGCTAAGGAACGTCC | UGGACCUGCUAAGCUAAGGAACG |
| | | | | CTTGAACTCCTTCTCAGGCCGGGA | UCCCUUGAACUCCUUCUCAGGCCG |
| | | | | CCTCTCTCGCCGCACTGAGGGGCA | GGACCUCUCUCGCCGCACUGAGG |
| | | | | CTCCACACCACGGGGGCC | GGCACUCCACACCACGGGGGCC |
| | | | | [SEQ ID NO: 548] | [SEQ ID NO: 1115] |
| AGGAACGUGGGUU | XD-14857 | miR-155E | 911 Control | CTGGAGGCTTGCTTTGGGCTGTAT | CUGGAGGCUUGCUUUGGGCUGUA |
| GAACUCCUU [SEQ | | | | GCTGAGGAACGTCCCTTGAACTCC | UGCUGAGGAACGUCCCUUGAACU |
| ID NO: 242] | | | | TTTTTTGGCCTCTGACTGAAAGGA | CCUUUUUUGGCCUCUGACUGAAA |
| | | | | GTTAAGGACGTTCCTCAGGACAAG | GGAGUUAAGGACGUUCCUCAGGA |
| | | | | GCCCTTTATCAGCACTCACATGGA | CAAGGCCCUUUAUCAGCACUCAC |
| | | | | ACAAATGGCCACCGTG | AUGGAACAAAUGGCCACCGUG |
| | | | | [SEQ ID NO: 549] | [SEQ ID NO: 1116] |
| UUCGGGUUGAAAU | XD-14790 | miR-155E | 911 Control | CTGGAGGCTTGCTTTGGGCTGTAT | CUGGAGGCUUGCUUUGGGCUGUA |
| CUGAAGUGU [SEQ | | | | GCTGTTCGGGTTCTTATCTGAAGTG | UGCUGUUCGGGUUCUUAUCUGAA |
| ID NO: 108] | | | | TTTTTGGCCTCTGACTGAACACTTC | GUGUUUUUGGCCUCUGACUGAAC |
| | | | | AATAGAACCCGAACAGGACAAGG | ACUUCAAUAGAACCCGAACAGGA |
| | | | | CCCTTTATCAGCACTCACATGGAA | CAAGGCCCUUUAUCAGCACUCAC |
| | | | | CAAATGGCCACCGTG | AUGGAACAAAUGGCCACCGUG |
| | | | | [SEQ ID NO: 550] | [SEQ ID NO: 1117] |
| UUCGGGUUGAAAU | XD-14790 | miR-1-1 | 911 Control | CATGCAGACTGCCTGCTTGGGAGA | CAUGCAGACUGCCUGCUUGGGAG |
| CUGAAGUGU [SEQ | | | | CTTCAGATAAGAACCGAGAATATG | ACUUCAGAUAAGAACCGAGAAUA |
| ID NO: 108] | | | | GACCTGCTAAGCTATTCGGGTTCTT | UGGACCUGCUAAGCUAUUCGGGU |
| | | | | ATCTGAAGTGTCTCAGGCCGGGAC | UCUUAUCUGAAGUGUCUCAGGCC |
| | | | | CTCTCTCGCCGCACTGAGGGGCAC | GGGACCUCUCUCGCCGCACUGAG |
| | | | | TCCACACCACGGGGGCC | GGGCACUCCACACCACGGGGGCC |
| | | | | [SEQ ID NO: 551] | [SEQ ID NO: 1118] |
| UUGAUUUCGAGGA | XD-14800 | miR-155E | 911 Control | CTGGAGGCTTGCTTTGGGCTGTAT | CUGGAGGCUUGCUUUGGGCUGUA |
| UGUCGCUGG [SEQ | | | | GCTGTTGATTTCCTCGATGTCGCTG | UGCUGUUGAUUUCCUCGAUGUCG |
| ID NO: 128] | | | | GTTTTGGCCTCTGACTGACCAGCG | CUGGUUUUGGCCUCUGACUGACC |
| | | | | ACTCGGGAAATCAACAGGACAAG | AGCGACUCGGGAAAUCAACAGGA |
| | | | | GCCCTTTATCAGCACTCACATGGA | CAAGGCCCUUUAUCAGCACUCAC |
| | | | | ACAAATGGCCACCGTG | AUGGAACAAAUGGCCACCGUG |
| | | | | [SEQ ID NO: 552] | [SEQ ID NO: 1119] |
| UUGAUUUCGAGGA | XD-14800 | miR-1-1 | 911 Control | CATGCAGACTGCCTGCTTGGGCGA | CAUGCAGACUGCCUGCUUGGGCG |
| UGUCGCUGG [SEQ | | | | GCGACATCGAGGAAACGCAATATG | AGCGACAUCGAGGAAACGCAAUA |
| ID NO: 128] | | | | GACCTGCTAAGCTATTGATTTCCTC | UGGACCUGCUAAGCUAUUGAUUU |
| | | | | GATGTCGCTGGCTCAGGCCGGGAC | CCUCGAUGUCGCUGGCUCAGGCC |
| | | | | CTCTCTCGCCGCACTGAGGGGCAC | GGGACCUCUCUCGCCGCACUGAG |
| | | | | TCCACACCACGGGGGCC | GGGCACUCCACACCACGGGGGCC |
| | | | | [SEQ ID NO: 553] | [SEQ ID NO: 1120] |
| AGAAAUCGUAGAC | XD-14743 | miR-1-1 | Atxn2 | CATGCAGACTGCCTGCTTGGGAGT | CAUGCAGACUGCCUGCUUGGGAG |
| UGAGGCAGU [SEQ | | | targeting | GCCTCAGTCTACGATCGTCTTATGG | UGCCUCAGUCUACGAUCGUCUUA |
| ID NO: 14] | | | | ACCTGCTAAGCTAAGAAATCGTAG | UGGACCUGCUAAGCUAAGAAAUC |
| | | | | ACTGAGGCAGTCTCAGGCCGGGAC | GUAGACUGAGGCAGUCUCAGGCC |
| | | | | CTCTCTCGCCGCACTGAGGGGCAC | GGGACCUCUCUCGCCGCACUGAG |
| | | | | TCCACACCACGGGGGCC | GGGCACUCCACACCACGGGGGCC |
| | | | | [SEQ ID NO: 554] | [SEQ ID NO: 1121] |
| AGAAAUCGUAGAC | XD-14743 | miR-155E | Atxn2 | CTGGAGGCTTGCTTTGGGCTGTAT | CUGGAGGCUUGCUUUGGGCUGUA |
| UGAGGCAGU [SEQ | | | targeting | GCTGAGAAATCGTAGACTGAGGCA | UGCUGAGAAAUCGUAGACUGAGG |
| ID NO: 14] | | | | GTTTTTGGCCTCTGACTGAACTGCC | CAGUUUUUGGCCUCUGACUGAAC |
| | | | | TCGTCACGATTTCTCAGGACAAGG | UGCCUCGUCACGAUUUCUCAGGA |
| | | | | CCCTTTATCAGCACTCACATGGAA | CAAGGCCCUUUAUCAGCACUCAC |
| | | | | CAAATGGCCACCGTG | AUGGAACAAAUGGCCACCGUG |
| | | | | [SEQ ID NO: 555] | [SEQ ID NO: 1122] |
| AGAUACGUCAUUU | XD-14766 | miR-1-1 | Atxn2 | CATGCAGACTGCCTGCTTGGGGCC | CAUGCAGACUGCCUGCUUGGGGC |
| UCCAAAGCC [SEQ | | | targeting | TTTGGAAAATGACGTCCTCTTATG | CUUUGGAAAAUGACGUCCUCUUA |
| ID NO: 60] | | | | GACCTGCTAAGCTAAGATACGTCA | UGGACCUGCUAAGCUAAGAUACG |
| | | | | TTTTCCAAAGCCCTCAGGCCGGGA | UCAUUUUCCAAAGCCCUCAGGCC |
| | | | | CCTCTCTCGCCGCACTGAGGGGCA | GGGACCUCUCUCGCCGCACUGAG |
| | | | | CTCCACACCACGGGGGCC | GGGCACUCCACACCACGGGGGCC |
| | | | | [SEQ ID NO: 556] | [SEQ ID NO: 1123] |
| AGAUACGUCAUUU | XD-14766 | miR-155E | Atxn2 | CTGGAGGCTTGCTTTGGGCTGTAT | CUGGAGGCUUGCUUUGGGCUGUA |
| UCCAAAGCC | | | targeting | GCTGAGATACGTCATTTTCCAAAG | UGCUGAGAUACGUCAUUUUCCAA |
| [SEQ ID NO: 60] | | | | CCTTTTGGCCTCTGACTGAGGCTTT | AGCCUUUUGGCCUCUGACUGAGG |
| | | | | GGAAAGACGTATCTCAGGACAAGG | CUUUGGAAAGACGUAUCUCAGGA |
| | | | | CCCTTTATCAGCACTCACATGGAA | CAAGGCCCUUUAUCAGCACUCAC |
| | | | | CAAATGGCCACCGTG | AUGGAACAAAUGGCCACCGUG |
| | | | | [SEQ ID NO: 557] | [SEQ ID NO: 1124] |
| AGCGUUAGGGUGC | XD-14904 | miR-155E | Atxn2 | CTGGAGGCTTGCTTTGGGCTGTAT | CUGGAGGCUUGCUUUGGGCUGUA |
| GCAUACUGC [SEQ | | | targeting | GCTGAGCGTTAGGGTGCGCATACT | UGCUGAGCGUUAGGGUGCGCAUA |
| ID NO: 336] | | | | GCTTTTGGCCTCTGACTGAGCAGT | CUGCUUUUGGCCUCUGACUGAGC |
| | | | | ATGGCACCTAACGCTCAGGACAAG | AGUAUGGCACCUAACGCUCAGGA |
| | | | | GCCCTTTATCAGCACTCACATGGA | CAAGGCCCUUUAUCAGCACUCAC |
| | | | | ACAAATGGCCACCGTG | AUGGAACAAAUGGCCACCGUG |
| | | | | [SEQ ID NO: 558] | [SEQ ID NO: 1125] |
| AGCGUUAGGGUGC | XD-14904 | miR-1-1 | Atxn2 | CATGCAGACTGCCTGCTTGGGGGA | CAUGCAGACUGCCUGCUUGGGGG |
| GCAUACUGC [SEQ | | | targeting | GTATGCGCACCCTAAGAGCTTATG | AGUAUGCGCACCCUAAGAGCUUA |
| ID NO: 336] | | | | GACCTGCTAAGCTAAGCGTTAGGG | UGGACCUGCUAAGCUAAGCGUUA |
| | | | | TGCGCATACTGCCTCAGGCCGGGA | GGGUGCGCAUACUGCCUCAGGCC |
| | | | | CCTCTCTCGCCGCACTGAGGGGCA | GGGACCUCUCUCGCCGCACUGAG |
| | | | | CTCCACACCACGGGGGCC | GGGCACUCCACACCACGGGGGCC |
| | | | | [SEQ ID NO: 559] | [SEQ ID NO: 1126] |
| AGGAACGUGGGUU | XD-14857 | miR-155E | Atxn2 | CTGGAGGCTTGCTTTGGGCTGTAT | CUGGAGGCUUGCUUUGGGCUGUA |
| GAACUCCUU [SEQ | | | targeting | GCTGAGGAACGTGGGTTGAACTCC | UGCUGAGGAACGUGGGUUGAACU |
| ID NO: 242] | | | | TTTTTTGGCCTCTGACTGAAAGGA | CCUUUUUUGGCCUCUGACUGAAA |
| | | | | GTTAACCACGTTCCTCAGGACAAG | GGAGUUAACCACGUUCCUCAGGA |
| | | | | GCCCTTTATCAGCACTCACATGGA | CAAGGCCCUUUAUCAGCACUCAC |
| | | | | ACAAATGGCCACCGTG | AUGGAACAAAUGGCCACCGUG |
| | | | | [SEQ ID NO: 560] | [SEQ ID NO: 1127] |
| AGGAACGUGGGUU | XD-14857 | miR-1-1 | Atxn2 | CATGCAGACTGCCTGCTTGGGATG | CAUGCAGACUGCCUGCUUGGGAU |
| GAACUCCUU [SEQ | | | targeting | GAGTTCAACCCACGTCGCCTTATG | GGAGUUCAACCCACGUCGCCUUA |
| ID NO: 242] | | | | GACCTGCTAAGCTAAGGAACGTGG | UGGACCUGCUAAGCUAAGGAACG |
| | | | | GTTGAACTCCTTCTCAGGCCGGGA | UGGGUUGAACUCCUUCUCAGGCC |
| | | | | CCTCTCTCGCCGCACTGAGGGGCA | GGGACCUCUCUCGCCGCACUGAG |
| | | | | CTCCACACCACGGGGGCC | GGGCACUCCACACCACGGGGGCC |
| | | | | [SEQ ID NO: 561] | [SEQ ID NO: 1128] |
| AUAAUAAUCCGUC | XD-14949 | miR-155E | Atxn2 | CTGGAGGCTTGCTTTGGGCTGTAT | CUGGAGGCUUGCUUUGGGCUGUA |
| AGUUUGACG [SEQ | | | targeting | GCTGATAATAATCCGTCAGTTTGA | UGCUGAUAAUAAUCCGUCAGUUU |
| ID NO: 426] | | | | CGTTTTGGCCTCTGACTGACGTCAA | GACGUUUUGGCCUCUGACUGACG |
| | | | | ACGACGATTATTATCAGGACAAGG | UCAAACGACGAUUAUUAUCAGGA |
| | | | | CCCTTTATCAGCACTCACATGGAA | CAAGGCCCUUUAUCAGCACUCAC |
| | | | | CAAATGGCCACCGTG | AUGGAACAAAUGGCCACCGUG |
| | | | | [SEQ ID NO: 562] | [SEQ ID NO: 1129] |
| AUAAUAAUCCGUC | XD-14949 | miR-1-1 | Atxn2 | CATGCAGACTGCCTGCTTGGGCCT | CAUGCAGACUGCCUGCUUGGGCC |
| AGUUUGACG [SEQ | | | targeting | CAAACTGACGGATTACGTATTATG | UCAAACUGACGGAUUACGUAUUA |
| ID NO: 426] | | | | GACCTGCTAAGCTAATAATAATCC | UGGACCUGCUAAGCUAAUAAUAA |
| | | | | GTCAGTTTGACGCTCAGGCCGGGA | UCCGUCAGUUUGACGCUCAGGCC |
| | | | | CCTCTCTCGCCGCACTGAGGGGCA | GGGACCUCUCUCGCCGCACUGAG |
| | | | | CTCCACACCACGGGGGCC | GGGCACUCCACACCACGGGGGCC |
| | | | | [SEQ ID NO: 563] | [SEQ ID NO: 1130] |
| AUACGCGGUGAAU | XD-14787 | miR-155E | Atxn2 | CTGGAGGCTTGCTTTGGGCTGTAT | CUGGAGGCUUGCUUUGGGCUGUA |
| UCUGUCUCC [SEQ | | | targeting | GCTGATACGCGGTGAATTCTGTCT | UGCUGAUACGCGGUGAAUUCUGU |
| ID NO: 102] | | | | CCTTTTGGCCTCTGACTGAGGAGA | CUCCUUUUGGCCUCUGACUGAGG |
| | | | | CAGATTACCGCGTATCAGGACAAG | AGACAGAUUACCGCGUAUCAGGA |
| | | | | GCCCTTTATCAGCACTCACATGGA | CAAGGCCCUUUAUCAGCACUCAC |
| | | | | ACAAATGGCCACCGTG | AUGGAACAAAUGGCCACCGUG |
| | | | | [SEQ ID NO: 564] | [SEQ ID NO: 1131] |
| AUACGCGGUGAAU | XD-14787 | miR-1-1 | Atxn2 | CATGCAGACTGCCTGCTTGGGGCA | CAUGCAGACUGCCUGCUUGGGGC |
| UCUGUCUCC [SEQ | | | targeting | GACAGAATTCACCGCCTTATTATG | AGACAGAAUUCACCGCCUUAUUA |
| ID NO: 102] | | | | GACCTGCTAAGCTAATACGCGGTG | UGGACCUGCUAAGCUAAUACGCG |
| | | | | AATTCTGTCTCCCTCAGGCCGGGA | GUGAAUUCUGUCUCCCUCAGGCC |
| | | | | CCTCTCTCGCCGCACTGAGGGGCA | GGGACCUCUCUCGCCGCACUGAG |
| | | | | CTCCACACCACGGGGGCC | GGGCACUCCACACCACGGGGGCC |
| | | | | [SEQ ID NO: 565] | [SEQ ID NO: 1132] |
| AUUAACUACUCUU | XD-14792 | miR-1-1 | Atxn2 | CATGCAGACTGCCTGCTTGGGTAC | CAUGCAGACUGCCUGCUUGGGUA |
| UGGUCUGAA [SEQ | | | targeting | AGACCAAAGAGTAGTCGAATTATG | CAGACCAAAGAGUAGUCGAAUUA |
| ID NO: 112] | | | | GACCTGCTAAGCTAATTAACTACT | UGGACCUGCUAAGCUAAUUAACU |
| | | | | CTTTGGTCTGAACTCAGGCCGGGA | ACUCUUUGGUCUGAACUCAGGCC |
| | | | | CCTCTCTCGCCGCACTGAGGGGCA | GGGACCUCUCUCGCCGCACUGAG |
| | | | | CTCCACACCACGGGGGCC | GGGCACUCCACACCACGGGGGCC |
| | | | | [SEQ ID NO: 538] | [SEQ ID NO: 1133] |
| AUUAACUACUCUU | XD-14792 | miR-155E | Atxn2 | CTGGAGGCTTGCTTTGGGCTGTAT | CUGGAGGCUUGCUUUGGGCUGUA |
| UGGUCUGAA [SEQ | | | targeting | GCTGATTAACTACTCTTTGGTCTGA | UGCUGAUUAACUACUCUUUGGUC |
| ID NO: 112] | | | | ATTTTGGCCTCTGACTGATTCAGAC | UGAAUUUUGGCCUCUGACUGAUU |
| | | | | CAAGGTAGTTAATCAGGACAAGGC | CAGACCAAGGUAGUUAAUCAGGA |
| | | | | CCTTTATCAGCACTCACATGGAAC | CAAGGCCCUUUAUCAGCACUCAC |
| | | | | AAATGGCCACCGTG | AUGGAACAAAUGGCCACCGUG |
| | | | | [SEQ ID NO: 566] | [SEQ ID NO: 1134] |
| AUUGCGUGGAGUA | XD-14889 | miR-155E | Atxn2 | CTGGAGGCTTGCTTTGGGCTGTAT | CUGGAGGCUUGCUUUGGGCUGUA |
| AGCUGGUGG [SEQ | | | targeting | GCTGATTGCGTGGAGTAAGCTGGT | UGCUGAUUGCGUGGAGUAAGCUG |
| ID NO: 306] | | | | GGTTTTGGCCTCTGACTGACCACC | GUGGUUUUGGCCUCUGACUGACC |
| | | | | AGCTACCCACGCAATCAGGACAAG | ACCAGCUACCCACGCAAUCAGGAC |
| | | | | GCCCTTTATCAGCACTCACATGGA | AAGGCCCUUUAUCAGCACUCACA |
| | | | | ACAAATGGCCACCGTG | UGGAACAAAUGGCCACCGUG |
| | | | | [SEQ ID NO: 567] | [SEQ ID NO: 1135] |
| AUUGCGUGGAGUA | XD-14889 | miR-1-1 | Atxn2 | CATGCAGACTGCCTGCTTGGGCGA | CAUGCAGACUGCCUGCUUGGGCG |
| AGCUGGUGG [SEQ | | | targeting | CCAGCTTACTCCACGGAAATTATG | ACCAGCUUACUCCACGGAAAUUA |
| ID NO: 306] | | | | GACCTGCTAAGCTAATTGCGTGGA | UGGACCUGCUAAGCUAAUUGCGU |
| | | | | GTAAGCTGGTGGCTCAGGCCGGGA | GGAGUAAGCUGGUGGCUCAGGCC |
| | | | | CCTCTCTCGCCGCACTGAGGGGCA | GGGACCUCUCUCGCCGCACUGAG |
| | | | | CTCCACACCACGGGGGCC | GGGCACUCCACACCACGGGGGCC |
| | | | | [SEQ ID NO: 568] | [SEQ ID NO: 1136] |
| AUUUCGAGGAUGU | XD-14798 | miR-155E | Atxn2 | CTGGAGGCTTGCTTTGGGCTGTAT | CUGGAGGCUUGCUUUGGGCUGUA |
| CGCUGGGCC [SEQ | | | targeting | GCTGATTTCGAGGATGTCGCTGGG | UGCUGAUUUCGAGGAUGUCGCUG |
| ID NO: 124] | | | | CCTTTTGGCCTCTGACTGAGGCCCA | GGCCUUUUGGCCUCUGACUGAGG |
| | | | | GCACACCTCGAAATCAGGACAAGG | CCCAGCACACCUCGAAAUCAGGAC |
| | | | | CCCTTTATCAGCACTCACATGGAA | AAGGCCCUUUAUCAGCACUCACA |
| | | | | CAAATGGCCACCGTG | UGGAACAAAUGGCCACCGUG |
| | | | | [SEQ ID NO: 569] | [SEQ ID NO: 1137] |
| AUUUCGAGGAUGU | XD-14798 | miR-1-1 | Atxn2 | CATGCAGACTGCCTGCTTGGGGCC | CAUGCAGACUGCCUGCUUGGGGC |
| CGCUGGGCC [SEQ | | | targeting | CCAGCGACATCCTCGCCAATTATG | CCCAGCGACAUCCUCGCCAAUUAU |
| ID NO: 124] | | | | GACCTGCTAAGCTAATTTCGAGGA | GGACCUGCUAAGCUAAUUUCGAG |
| | | | | TGTCGCTGGGCCCTCAGGCCGGGA | GAUGUCGCUGGGCCCUCAGGCCG |
| | | | | CCTCTCTCGCCGCACTGAGGGGCA | GGACCUCUCUCGCCGCACUGAGG |
| | | | | CTCCACACCACGGGGGCC | GGCACUCCACACCACGGGGGCC |
| | | | | [SEQ ID NO: 570] | [SEQ ID NO: 1138] |
| UAAAUCGUAGACU | XD-14742 | miR-1-1 | Atxn2 | CATGCAGACTGCCTGCTTGGGGTC | UAGACUGAGGCAGUCCUCAGGCC |
| GAGGCAGUC [SEQ | | | targeting | TGCCTCAGTCTACGACGTTATATG | CAUGCAGACUGCCUGCUUGGGGU |
| ID NO: 12] | | | | GACCTGCTAAGCTATAAATCGTAG | CUGCCUCAGUCUACGACGUUAUA |
| | | | | ACTGAGGCAGTCCTCAGGCCGGGA | UGGACCUGCUAAGCUAUAAAUCG |
| | | | | CCTCTCTCGCCGCACTGAGGGGCA | GGGACCUCUCUCGCCGCACUGAG |
| | | | | CTCCACACCACGGGGGCC | GGGCACUCCACACCACGGGGGCC |
| | | | | [SEQ ID NO: 571] | [SEQ ID NO: 1139] |
| UAAAUCGUAGACU | XD-14742 | miR-155E | Atxn2 | CTGGAGGCTTGCTTTGGGCTGTAT | CUGGAGGCUUGCUUUGGGCUGUA |
| GAGGCAGUC [SEQ | | | targeting | GCTGTAAATCGTAGACTGAGGCAG | UGCUGUAAAUCGUAGACUGAGGC |
| ID NO: 12] | | | | TCTTTTGGCCTCTGACTGAGACTGC | AGUCUUUUGGCCUCUGACUGAGA |
| | | | | CTAGTTACGATTTACAGGACAAGG | CUGCCUAGUUACGAUUUACAGGA |
| | | | | CCCTTTATCAGCACTCACATGGAA | CAAGGCCCUUUAUCAGCACUCAC |
| | | | | CAAATGGCCACCGTG | AUGGAACAAAUGGCCACCGUG |
| | | | | [SEQ ID NO: 572] | [SEQ ID NO: 1140] |
| UACGCGGUGAAUU | XD-14786 | miR-1-1 | Atxn2 | CATGCAGACTGCCTGCTTGGGGCG | CAUGCAGACUGCCUGCUUGGGGC |
| CUGUCUCCC [SEQ | | | targeting | AGACAGAATTCACCGGAGTATATG | GAGACAGAAUUCACCGGAGUAUA |
| ID NO: 100] | | | | GACCTGCTAAGCTATACGCGGTGA | UGGACCUGCUAAGCUAUACGCGG |
| | | | | ATTCTGTCTCCCCTCAGGCCGGGA | UGAAUUCUGUCUCCCCUCAGGCC |
| | | | | CCTCTCTCGCCGCACTGAGGGGCA | GGGACCUCUCUCGCCGCACUGAG |
| | | | | CTCCACACCACGGGGGCC | GGGCACUCCACACCACGGGGGCC |
| | | | | [SEQ ID NO: 573] | [SEQ ID NO: 1141] |
| UACGCGGUGAAUU | XD-14786 | miR-155E | Atxn2 | CTGGAGGCTTGCTTTGGGCTGTAT | CUGGAGGCUUGCUUUGGGCUGUA |
| CUGUCUCCC [SEQ | | | targeting | GCTGTACGCGGTGAATTCTGTCTCC | UGCUGUACGCGGUGAAUUCUGUC |
| ID NO: 100] | | | | CTTTTGGCCTCTGACTGAGGGAGA | UCCCUUUUGGCCUCUGACUGAGG |
| | | | | CAAATCACCGCGTACAGGACAAGG | GAGACAAAUCACCGCGUACAGGA |
| | | | | CCCTTTATCAGCACTCACATGGAA | CAAGGCCCUUUAUCAGCACUCAC |
| | | | | CAAATGGCCACCGTG | AUGGAACAAAUGGCCACCGUG |
| | | | | [SEQ ID NO: 574] | [SEQ ID NO: 1142] |
| UAUACGCGGUGAA | XD-14788 | miR-1-1 | Atxn2 | CATGCAGACTGCCTGCTTGGGGTG | CAUGCAGACUGCCUGCUUGGGGU |
| UUCUGUCUC [SEQ | | | targeting | ACAGAATTCACCGCGCGATATATG | GACAGAAUUCACCGCGCGAUAUA |
| ID NO: 104] | | | | GACCTGCTAAGCTATATACGCGGT | UGGACCUGCUAAGCUAUAUACGC |
| | | | | GAATTCTGTCTCCTCAGGCCGGGA | GGUGAAUUCUGUCUCCUCAGGCC |
| | | | | CCTCTCTCGCCGCACTGAGGGGCA | GGGACCUCUCUCGCCGCACUGAG |
| | | | | CTCCACACCACGGGGGCC | GGGCACUCCACACCACGGGGGCC |
| | | | | [SEQ ID NO: 575] | [SEQ ID NO: 1143] |
| UAUACGCGGUGAA | XD-14788 | miR-155E | Atxn2 | CTGGAGGCTTGCTTTGGGCTGTAT | CUGGAGGCUUGCUUUGGGCUGUA |
| UUCUGUCUC [SEQ | | | targeting | GCTGTATACGCGGTGAATTCTGTCT | UGCUGUAUACGCGGUGAAUUCUG |
| ID NO: 104] | | | | CTTTTGGCCTCTGACTGAGAGACA | UCUCUUUUGGCCUCUGACUGAGA |
| | | | | GATTCCCGCGTATACAGGACAAGG | GACAGAUUCCCGCGUAUACAGGA |
| | | | | CCCTTTATCAGCACTCACATGGAA | CAAGGCCCUUUAUCAGCACUCAC |
| | | | | CAAATGGCCACCGTG | AUGGAACAAAUGGCCACCGUG |
| | | | | [SEQ ID NO: 576] | [SEQ ID NO: 1144] |
| UAUUGCGUGGAGU | XD-14890 | miR-1-1 | Atxn2 | CATGCAGACTGCCTGCTTGGGCTC | CAUGCAGACUGCCUGCUUGGGCU |
| AAGCUGGUG [SEQ | | | targeting | CAGCTTACTCCACGCCCATATATG | CCAGCUUACUCCACGCCCAUAUAU |
| ID NO: 308] | | | | GACCTGCTAAGCTATATTGCGTGG | GGACCUGCUAAGCUAUAUUGCGU |
| | | | | AGTAAGCTGGTGCTCAGGCCGGGA | GGAGUAAGCUGGUGCUCAGGCCG |
| | | | | CCTCTCTCGCCGCACTGAGGGGCA | GGACCUCUCUCGCCGCACUGAGG |
| | | | | CTCCACACCACGGGGGCC | GGCACUCCACACCACGGGGGCC |
| | | | | [SEQ ID NO: 577] | [SEQ ID NO: 1145] |
| UAUUGCGUGGAGU | XD-14890 | miR-155E | Atxn2 | CTGGAGGCTTGCTTTGGGCTGTAT | CUGGAGGCUUGCUUUGGGCUGUA |
| AAGCUGGUG [SEQ | | | targeting | GCTGTATTGCGTGGAGTAAGCTGG | UGCUGUAUUGCGUGGAGUAAGCU |
| ID NO: 308] | | | | TGTTTTGGCCTCTGACTGACACCAG | GGUGUUUUGGCCUCUGACUGACA |
| | | | | CTACTCACGCAATACAGGACAAGG | CCAGCUACUCACGCAAUACAGGA |
| | | | | CCCTTTATCAGCACTCACATGGAA | CAAGGCCCUUUAUCAGCACUCAC |
| | | | | CAAATGGCCACCGTG | AUGGAACAAAUGGCCACCGUG |
| | | | | [SEQ ID NO: 578] | [SEQ ID NO: 1146] |
| UAUUUCGAGGAUG | XD-14799 | miR-155E | Atxn2 | CTGGAGGCTTGCTTTGGGCTGTAT | CUGGAGGCUUGCUUUGGGCUGUA |
| UCGCUGGGC [SEQ | | | targeting | GCTGTATTTCGAGGATGTCGCTGG | UGCUGUAUUUCGAGGAUGUCGCU |
| ID NO: 126] | | | | GCTTTTGGCCTCTGACTGAGCCCA | GGGCUUUUGGCCUCUGACUGAGC |
| | | | | GCGCATCTCGAAATACAGGACAAG | CCAGCGCAUCUCGAAAUACAGGA |
| | | | | GCCCTTTATCAGCACTCACATGGA | CAAGGCCCUUUAUCAGCACUCAC |
| | | | | ACAAATGGCCACCGTG | AUGGAACAAAUGGCCACCGUG |
| | | | | [SEQ ID NO: 579] | [SEQ ID NO: 1147] |
| UAUUUCGAGGAUG | XD-14799 | miR-1-1 | Atxn2 | CATGCAGACTGCCTGCTTGGGGGC | CAUGCAGACUGCCUGCUUGGGGG |
| UCGCUGGGC [SEQ | | | targeting | CAGCGACATCCTCGACCATATATG | CCAGCGACAUCCUCGACCAUAUA |
| ID NO: 126] | | | | GACCTGCTAAGCTATATTTCGAGG | UGGACCUGCUAAGCUAUAUUUCG |
| | | | | ATGTCGCTGGGCCTCAGGCCGGGA | AGGAUGUCGCUGGGCCUCAGGCC |
| | | | | CCTCTCTCGCCGCACTGAGGGGCA | GGGACCUCUCUCGCCGCACUGAG |
| | | | | CTCCACACCACGGGGGCC | GGGCACUCCACACCACGGGGGCC |
| | | | | [SEQ ID NO: 580] | [SEQ ID NO: 1148] |
| UCGCUGUUGGGGC | XD-14887 | miR-1-1 | Atxn2 | CATGCAGACTGCCTGCTTGGGAGC | CAUGCAGACUGCCUGCUUGGGAG |
| AUAUUUGGU [SEQ | | | targeting | AAATATGCCCCAACACTCGATATG | CAAAUAUGCCCCAACACUCGAUA |
| ID NO: 302] | | | | GACCTGCTAAGCTATCGCTGTTGG | UGGACCUGCUAAGCUAUCGCUGU |
| | | | | GGCATATTTGGTCTCAGGCCGGGA | UGGGGCAUAUUUGGUCUCAGGCC |
| | | | | CCTCTCTCGCCGCACTGAGGGGCA | GGGACCUCUCUCGCCGCACUGAG |
| | | | | CTCCACACCACGGGGGCC | GGGCACUCCACACCACGGGGGCC |
| | | | | [SEQ ID NO: 581] | [SEQ ID NO: 1149] |
| UCGCUGUUGGGGC | XD-14887 | miR-155E | Atxn2 | CTGGAGGCTTGCTTTGGGCTGTAT | CUGGAGGCUUGCUUUGGGCUGUA |
| AUAUUUGGU [SEQ | | | targeting | GCTGTCGCTGTTGGGGCATATTTG | UGCUGUCGCUGUUGGGGCAUAUU |
| ID NO: 302] | | | | GTTTTTGGCCTCTGACTGAACCAA | UGGUUUUUGGCCUCUGACUGAAC |
| | | | | ATAGCCCAACAGCGACAGGACAA | CAAAUAGCCCAACAGCGACAGGA |
| | | | | GGCCCTTTATCAGCACTCACATGG | CAAGGCCCUUUAUCAGCACUCAC |
| | | | | AACAAATGGCCACCGTG | AUGGAACAAAUGGCCACCGUG |
| | | | | [SEQ ID NO: 582] | [SEQ ID NO: 1150] |
| UGCGCAUACUGCUG | XD-14901 | miR-1-1 | Atxn2 | CATGCAGACTGCCTGCTTGGGCGC | CAUGCAGACUGCCUGCUUGGGCG |
| AGCAAGGG [SEQ ID | | | targeting | TTGCTCAGCAGTATGGAGCATATG | CUUGCUCAGCAGUAUGGAGCAUA |
| NO: 330] | | | | GACCTGCTAAGCTATGCGCATACT | UGGACCUGCUAAGCUAUGCGCAU |
| | | | | GCTGAGCAAGGGCTCAGGCCGGGA | ACUGCUGAGCAAGGGCUCAGGCC |
| | | | | CCTCTCTCGCCGCACTGAGGGGCA | GGGACCUCUCUCGCCGCACUGAG |
| | | | | CTCCACACCACGGGGGCC | GGGCACUCCACACCACGGGGGCC |
| | | | | [SEQ ID NO: 583] | [SEQ ID NO: 1151] |
| UGCGCAUACUGCUG | XD-14901 | miR-155E | Atxn2 | CTGGAGGCTTGCTTTGGGCTGTAT | CUGGAGGCUUGCUUUGGGCUGUA |
| AGCAAGGG [SEQ ID | | | targeting | GCTGTGCGCATACTGCTGAGCAAG | UGCUGUGCGCAUACUGCUGAGCA |
| NO: 330] | | | | GGTTTTGGCCTCTGACTGACCCTTG | AGGGUUUUGGCCUCUGACUGACC |
| | | | | CTAGCGTATGCGCACAGGACAAGG | CUUGCUAGCGUAUGCGCACAGGA |
| | | | | CCCTTTATCAGCACTCACATGGAA | CAAGGCCCUUUAUCAGCACUCAC |
| | | | | CAAATGGCCACCGTG | AUGGAACAAAUGGCCACCGUG |
| | | | | [SEQ ID NO: 584] | [SEQ ID NO: 1152] |
| UGUACCACAACAAA | XD-14756 | miR-1-1 | Atxn2 | CATGCAGACTGCCTGCTTGGGGAT | CAUGCAGACUGCCUGCUUGGGGA |
| GUCUGAAC [SEQ ID | | | targeting | CAGACTTTGTTGTGGCGACATATG | UCAGACUUUGUUGUGGCGACAUA |
| NO: 40] | | | | GACCTGCTAAGCTATGTACCACAA | UGGACCUGCUAAGCUAUGUACCA |
| | | | | CAAAGTCTGAACCTCAGGCCGGGA | CAACAAAGUCUGAACCUCAGGCC |
| | | | | CCTCTCTCGCCGCACTGAGGGGCA | GGGACCUCUCUCGCCGCACUGAG |
| | | | | CTCCACACCACGGGGGCC | GGGCACUCCACACCACGGGGGCC |
| | | | | [SEQ ID NO: 585] | [SEQ ID NO: 1153] |
| UGUACCACAACAAA | XD-14756 | miR-155E | Atxn2 | CTGGAGGCTTGCTTTGGGCTGTAT | CUGGAGGCUUGCUUUGGGCUGUA |
| GUCUGAAC [SEQ ID | | | targeting | GCTGTGTACCACAACAAAGTCTGA | UGCUGUGUACCACAACAAAGUCU |
| NO: 40] | | | | ACTTTTGGCCTCTGACTGAGTTCAG | GAACUUUUGGCCUCUGACUGAGU |
| | | | | ACTTGTGTGGTACACAGGACAAGG | UCAGACUUGUGUGGUACACAGGA |
| | | | | CCCTTTATCAGCACTCACATGGAA | CAAGGCCCUUUAUCAGCACUCAC |
| | | | | CAAATGGCCACCGTG | AUGGAACAAAUGGCCACCGUG |
| | | | | [SEQ ID NO: 586] | [SEQ ID NO: 1154] |
| UGUAUACGCCGGCU | XD-14917 | miR-1-1 | Atxn2 | CATGCAGACTGCCTGCTTGGGTGA | CAUGCAGACUGCCUGCUUGGGUG |
| GAACGUGA [SEQ ID | | | targeting | CGTTCAGCCGGCGTACGACATATG | ACGUUCAGCCGGCGUACGACAUA |
| NO: 362] | | | | GACCTGCTAAGCTATGTATACGCC | UGGACCUGCUAAGCUAUGUAUAC |
| | | | | GGCTGAACGTGACTCAGGCCGGGA | GCCGGCUGAACGUGACUCAGGCC |
| | | | | CCTCTCTCGCCGCACTGAGGGGCA | GGGACCUCUCUCGCCGCACUGAG |
| | | | | CTCCACACCACGGGGGCC | GGGCACUCCACACCACGGGGGCC |
| | | | | [SEQ ID NO: 587] | [SEQ ID NO: 1155] |
| UGUAUACGCCGGCU | XD-14917 | miR-155E | Atxn2 | CTGGAGGCTTGCTTTGGGCTGTAT | CUGGAGGCUUGCUUUGGGCUGUA |
| GAACGUGA [SEQ ID | | | targeting | GCTGTGTATACGCCGGCTGAACGT | UGCUGUGUAUACGCCGGCUGAAC |
| NO: 362] | | | | GATTTTGGCCTCTGACTGATCACGT | GUGAUUUUGGCCUCUGACUGAUC |
| | | | | TCGCCGCGTATACACAGGACAAGG | ACGUUCGCCGCGUAUACACAGGA |
| | | | | CCCTTTATCAGCACTCACATGGAA | CAAGGCCCUUUAUCAGCACUCAC |
| | | | | CAAATGGCCACCGTG | AUGGAACAAAUGGCCACCGUG |
| | | | | [SEQ ID NO: 588] | [SEQ ID NO: 1156] |
| UUACUAAGUAUUG | XD-14846 | miR-155E | Atxn2 | CTGGAGGCTTGCTTTGGGCTGTAT | CUGGAGGCUUGCUUUGGGCUGUA |
| AAGGGGAAA [SEQ | | | targeting | GCTGTTACTAAGTATTGAAGGGGA | UGCUGUUACUAAGUAUUGAAGGG |
| ID NO: 220] | | | | AATTTTGGCCTCTGACTGATTTCCC | GAAAUUUUGGCCUCUGACUGAUU |
| | | | | CTCAAACTTAGTAACAGGACAAGG | UCCCCUCAAACUUAGUAACAGGA |
| | | | | CCCTTTATCAGCACTCACATGGAA | CAAGGCCCUUUAUCAGCACUCAC |
| | | | | CAAATGGCCACCGTG | AUGGAACAAAUGGCCACCGUG |
| | | | | [SEQ ID NO: 589] | [SEQ ID NO: 1157] |
| UUACUAAGUAUUG | XD-14846 | miR-1-1 | Atxn2 | CATGCAGACTGCCTGCTTGGGTAT | CAUGCAGACUGCCUGCUUGGGUA |
| AAGGGGAAA [SEQ | | | targeting | CCCCTTCAATACTTACTTAATATGG | UCCCCUUCAAUACUUACUUAAUA |
| ID NO: 220] | | | | ACCTGCTAAGCTATTACTAAGTATT | UGGACCUGCUAAGCUAUUACUAA |
| | | | | GAAGGGGAAACTCAGGCCGGGAC | GUAUUGAAGGGGAAACUCAGGCC |
| | | | | CTCTCTCGCCGCACTGAGGGGCAC | GGGACCUCUCUCGCCGCACUGAG |
| | | | | TCCACACCACGGGGGCC | GGGCACUCCACACCACGGGGGCC |
| | | | | [SEQ ID NO: 590] | [SEQ ID NO: 1158] |
| UUAGUUGAUCCAU | XD-14835 | miR-1-1 | Atxn2 | CATGCAGACTGCCTGCTTGGGTGT | CAUGCAGACUGCCUGCUUGGGUG |
| AGAUUCAGA [SEQ | | | targeting | GAATCTATGGATCAAGATAATATG | UGAAUCUAUGGAUCAAGAUAAUA |
| ID NO: 198] | | | | GACCTGCTAAGCTATTAGTTGATC | UGGACCUGCUAAGCUAUUAGUUG |
| | | | | CATAGATTCAGACTCAGGCCGGGA | AUCCAUAGAUUCAGACUCAGGCC |
| | | | | CCTCTCTCGCCGCACTGAGGGGCA | GGGACCUCUCUCGCCGCACUGAG |
| | | | | CTCCACACCACGGGGGCC | GGGCACUCCACACCACGGGGGCC |
| | | | | [SEQ ID NO: 591] | [SEQ ID NO: 1159] |
| UUAGUUGAUCCAU | XD-14835 | miR-155E | Atxn2 | CTGGAGGCTTGCTTTGGGCTGTAT | CUGGAGGCUUGCUUUGGGCUGUA |
| AGAUUCAGA [SEQ | | | targeting | GCTGTTAGTTGATCCATAGATTCA | UGCUGUUAGUUGAUCCAUAGAUU |
| ID NO: 198] | | | | GATTTTGGCCTCTGACTGATCTGAA | CAGAUUUUGGCCUCUGACUGAUC |
| | | | | TCATGATCAACTAACAGGACAAGG | UGAAUCAUGAUCAACUAACAGGA |
| | | | | CCCTTTATCAGCACTCACATGGAA | CAAGGCCCUUUAUCAGCACUCAC |
| | | | | CAAATGGCCACCGTG | AUGGAACAAAUGGCCACCGUG |
| | | | | [SEQ ID NO: 592] | [SEQ ID NO: 1160] |
| UUCGAUGCAGGAC | XD-14819 | miR-155E | Atxn2 | CTGGAGGCTTGCTTTGGGCTGTAT | CUGGAGGCUUGCUUUGGGCUGUA |
| UAGCAGGCG [SEQ | | | targeting | GCTGTTCGATGCAGGACTAGCAGG | UGCUGUUCGAUGCAGGACUAGCA |
| ID NO: 166] | | | | CGTTTTGGCCTCTGACTGACGCCTG | GGCGUUUUGGCCUCUGACUGACG |
| | | | | CTGTCTGCATCGAACAGGACAAGG | CCUGCUGUCUGCAUCGAACAGGA |
| | | | | CCCTTTATCAGCACTCACATGGAA | CAAGGCCCUUUAUCAGCACUCAC |
| | | | | CAAATGGCCACCGTG | AUGGAACAAAUGGCCACCGUG |
| | | | | [SEQ ID NO: 593] | [SEQ ID NO: 1161] |
| UUCGAUGCAGGAC | XD-14819 | miR-1-1 | Atxn2 | CATGCAGACTGCCTGCTTGGGCCC | CAUGCAGACUGCCUGCUUGGGCC |
| UAGCAGGCG [SEQ | | | targeting | CTGCTAGTCCTGCATGAGAATATG | CCUGCUAGUCCUGCAUGAGAAUA |
| ID NO: 166] | | | | GACCTGCTAAGCTATTCGATGCAG | UGGACCUGCUAAGCUAUUCGAUG |
| | | | | GACTAGCAGGCGCTCAGGCCGGGA | CAGGACUAGCAGGCGCUCAGGCC |
| | | | | CCTCTCTCGCCGCACTGAGGGGCA | GGGACCUCUCUCGCCGCACUGAG |
| | | | | CTCCACACCACGGGGGCC | GGGCACUCCACACCACGGGGGCC |
| | | | | [SEQ ID NO: 594] | [SEQ ID NO: 1162] |
| UUCGGGUUGAAAU | XD-14790 | miR-155E | Atxn2 | CTGGAGGCTTGCTTTGGGCTGTAT | CUGGAGGCUUGCUUUGGGCUGUA |
| CUGAAGUGU [SEQ | | | targeting | GCTGTTCGGGTTGAAATCTGAAGT | UGCUGUUCGGGUUGAAAUCUGAA |
| ID NO: 108] | | | | GTTTTTGGCCTCTGACTGAACACTT | GUGUUUUUGGCCUCUGACUGAAC |
| | | | | CAATTCAACCCGAACAGGACAAGG | ACUUCAAUUCAACCCGAACAGGA |
| | | | | CCCTTTATCAGCACTCACATGGAA | CAAGGCCCUUUAUCAGCACUCAC |
| | | | | CAAATGGCCACCGTG | AUGGAACAAAUGGCCACCGUG |
| | | | | [SEQ ID NO: 595] | [SEQ ID NO: 1163] |
| UUCGGGUUGAAAU | XD-14790 | miR-1-1 | Atxn2 | CATGCAGACTGCCTGCTTGGGAGA | CAUGCAGACUGCCUGCUUGGGAG |
| CUGAAGUGU [SEQ | | | targeting | CTTCAGATTTCAACCGAGAATATG | ACUUCAGAUUUCAACCGAGAAUA |
| ID NO: 108] | | | | GACCTGCTAAGCTATTCGGGTTGA | UGGACCUGCUAAGCUAUUCGGGU |
| | | | | AATCTGAAGTGTCTCAGGCCGGGA | UGAAAUCUGAAGUGUCUCAGGCC |
| | | | | CCTCTCTCGCCGCACTGAGGGGCA | GGGACCUCUCUCGCCGCACUGAG |
| | | | | CTCCACACCACGGGGGCC | GGGCACUCCACACCACGGGGGCC |
| | | | | [SEQ ID NO: 596] | [SEQ ID NO: 1164] |
| UUGAUUUCGAGGA | XD-14800 | miR-1-1 | Atxn2 | CATGCAGACTGCCTGCTTGGGCGA | CAUGCAGACUGCCUGCUUGGGCG |
| UGUCGCUGG [SEQ | | | targeting | GCGACATCCTCGAAACGCAATATG | AGCGACAUCCUCGAAACGCAAUA |
| ID NO: 128] | | | | GACCTGCTAAGCTATTGATTTCGA | UGGACCUGCUAAGCUAUUGAUUU |
| | | | | GGATGTCGCTGGCTCAGGCCGGGA | CGAGGAUGUCGCUGGCUCAGGCC |
| | | | | CCTCTCTCGCCGCACTGAGGGGCA | GGGACCUCUCUCGCCGCACUGAG |
| | | | | CTCCACACCACGGGGGCC | GGGCACUCCACACCACGGGGGCC |
| | | | | [SEQ ID NO: 597] | [SEQ ID NO: 1165] |
| UUGAUUUCGAGGA | XD-14800 | miR-155E | Atxn2 | CTGGAGGCTTGCTTTGGGCTGTAT | CUGGAGGCUUGCUUUGGGCUGUA |
| UGUCGCUGG [SEQ | | | targeting | GCTGTTGATTTCGAGGATGTCGCT | UGCUGUUGAUUUCGAGGAUGUCG |
| ID NO: 128] | | | | GGTTTTGGCCTCTGACTGACCAGC | CUGGUUUUGGCCUCUGACUGACC |
| | | | | GACTCCCGAAATCAACAGGACAAG | AGCGACUCCCGAAAUCAACAGGA |
| | | | | GCCCTTTATCAGCACTCACATGGA | CAAGGCCCUUUAUCAGCACUCAC |
| | | | | ACAAATGGCCACCGTG | AUGGAACAAAUGGCCACCGUG |
| | | | | [SEQ ID NO: 598] | [SEQ ID NO: 1166] |
| UUGUACUGGGCAC | XD-14781 | miR-155E | Atxn2 | CTGGAGGCTTGCTTTGGGCTGTAT | CUGGAGGCUUGCUUUGGGCUGUA |
| UUGACUCAA [SEQ | | | targeting | GCTGTTGTACTGGGCACTTGACTC | UGCUGUUGUACUGGGCACUUGAC |
| ID NO: 90] | | | | AATTTTGGCCTCTGACTGATTGAGT | UCAAUUUUGGCCUCUGACUGAUU |
| | | | | CAGTGCCAGTACAACAGGACAAGG | GAGUCAGUGCCAGUACAACAGGA |
| | | | | CCCTTTATCAGCACTCACATGGAA | CAAGGCCCUUUAUCAGCACUCAC |
| | | | | CAAATGGCCACCGTG | AUGGAACAAAUGGCCACCGUG |
| | | | | [SEQ ID NO: 599] | [SEQ ID NO: 1167] |
| UUGUACUGGGCAC | XD-14781 | miR-1-1 | Atxn2 | CATGCAGACTGCCTGCTTGGGTAG | CAUGCAGACUGCCUGCUUGGGUA |
| UUGACUCAA [SEQ | | | targeting | AGTCAAGTGCCCAGTCCCAATATG | GAGUCAAGUGCCCAGUCCCAAUA |
| ID NO: 90] | | | | GACCTGCTAAGCTATTGTACTGGG | UGGACCUGCUAAGCUAUUGUACU |
| | | | | CACTTGACTCAACTCAGGCCGGGA | GGGCACUUGACUCAACUCAGGCC |
| | | | | CCTCTCTCGCCGCACTGAGGGGCA | GGGACCUCUCUCGCCGCACUGAG |
| | | | | CTCCACACCACGGGGGCC | GGGCACUCCACACCACGGGGGCC |
| | | | | [SEQ ID NO: 600] | [SEQ ID NO: 1168] |

ATXN2-targeting miRNA guide sequences having at least 25% ATXN2 immunofluorescence signal knockdown are shown in Table 12 (both RNA and DNA versions). ATXN2-targeting miRNA guide sequences having at least 50% ATXN2 immunofluorescence signal knockdown are shown in Table 13 (both RNA and DNA versions).

TABLE 12
miRNA guide sequences with at least 25% knockdown of ATXN2

| siRNA | Guide (antisense sequence) | Guide (antisense sequence) |
|---|---|---|
| duplex ID | (RNA) | (DNA) |
| XD-14742 | UAAAUCGUAGACUGAGGCA | TAAATCGTAGACTGAGGCA |
| | GUC | GTC |
| | [SEQ ID NO: 12] | [SEQ ID NO: 601] |
| XD-14743 | AGAAAUCGUAGACUGAGGC | AGAAATCGTAGACTGAGGC |
| | AGU | AGT |
| | [SEQ ID NO: 14] | [SEQ ID NO: 602] |
| XD-14756 | UGUACCACAACAAAGUCUG | TGTACCACAACAAAGTCTGA |
| | AAC | AC |
| | [SEQ ID NO: 40] | [SEQ ID NO: 603] |
| XD-14766 | AGAUACGUCAUUUUCCAAA | AGATACGTCATTTTCCAAAG |
| | GCC | CC |
| | [SEQ ID NO: 601 | SEQ ID NO: 604] |
| XD-14786 | UACGCGGUGAAUUCUGUCU | TACGCGGTGAATTCTGTCTC |
| | CCC | CC |
| | [SEQ ID NO: 100] | [SEQ ID NO: 605] |
| XD-14788 | UAUACGCGGUGAAUUCUGU | TATACGCGGTGAATTCTGTC |
| | CUC | TC |
| | [SEQ ID NO: 104] | [SEQ ID NO: 606] |
| XD-14790 | UUCGGGUUGAAAUCUGAAG | TTCGGGTTGAAATCTGAAGT |
| | UGU | GT |
| | [SEQ ID NO: 108] | [SEQ ID NO: 607] |
| XD-14792 | AUUAACUACUCUUUGGUCU | ATTAACTACTCTTTGGTCTG |
| | GAA | AA |
| | [SEQ ID NO: 112] | [SEQ ID NO: 608] |
| XD-14798 | AUUUCGAGGAUGUCGCUGG | ATTTCGAGGATGTCGCTGGG |
| | GCC | CC |
| | [SEQ ID NO: 124 | [SEQ ID NO: 609] |
| XD-14799 | UAUUUCGAGGAUGUCGCUG | TATTTCGAGGATGTCGCTGG |
| | GGC | GC |
| | [SEQ ID NO: 126] | [SEQ ID NO: 610] |
| XD-14800 | UUGAUUUCGAGGAUGUCGC | TTGATTTCGAGGATGTCGCT |
| | UGG | GG |
| | [SEQ ID NO: 128] | [SEQ ID NO: 611] |
| XD-14819 | UUCGAUGCAGGACUAGCAG | TTCGATGCAGGACTAGCAGG |
| | GCG | CG |
| | [SEQ ID NO: 166] | [SEQ ID NO: 612] |
| XD-14835 | UUAGUUGAUCCAUAGAUUC | TTAGTTGATCCATAGATTCA |
| | AGA | GA |
| | [SEQ ID NO: 198] | [SEQ ID NO: 613] |
| XD-14846 | UUACUAAGUAUUGAAGGGG | TTACTAAGTATTGAAGGGGA |
| | AAA | AA |
| | [SEQ ID NO: 220] | [SEQ ID NO: 614] |
| XD-14857 | AGGAACGUGGGUUGAACUC | AGGAACGTGGGTTGAACTCC |
| | CUU | TT |
| | [SEQ ID NO: 242] | [SEQ ID NO: 615] |
| XD-14887 | UCGCUGUUGGGGCAUAUUU | TCGCTGTTGGGGCATATTTG |
| | GGU | GT |
| | [SEQ ID NO: 302] | [SEQ ID NO: 616] |
| XD-14889 | AUUGCGUGGAGUAAGCUGG | ATTGCGTGGAGTAAGCTGGT |
| | UGG | GG |
| | [SEQ ID NO: 306] | [SEQ ID NO: 617] |
| XD-14890 | UAUUGCGUGGAGUAAGCUG | TATTGCGTGGAGTAAGCTGG |
| | GUG | TG |
| | [SEQ ID NO: 308] | [SEQ ID NO: 618] |
| XD-14901 | UGCGCAUACUGCUGAGCAA | TGCGCATACTGCTGAGCAAG |
| | GGG | GG |
| | [SEQ ID NO: 330] | [SEQ ID NO: 619] |
| XD-14904 | AGCGUUAGGGUGCGCAUAC | AGCGTTAGGGTGCGCATACT |
| | UGC | GC |
| | [SEQ ID NO: 336] | [SEQ ID NO: 620] |
| XD-14917 | UGUAUACGCCGGCUGAACG | TGTATACGCCGGCTGAACGT |
| | UGA | GA |
| | [SEQ ID NO: 362] | [SEQ ID NO: 621] |

TABLE 13
miRNA sequences with at least 50% knockdown of ATXN2

| SiRNA | Guide (antisense sequence) | Guide (antisense sequence) |
|---|---|---|
| duplex ID | (RNA) | (DNA) |
| XD-14743 | AGAAAUCGUAGACUGAGGC | AGAAATCGTAGACTGAGGC |
| | AGU | AGT |
| | [SEQ ID NO: 14] | [SEQ ID NO: 602] |
| XD-14756 | UGUACCACAACAAAGUCUG | TGTACCACAACAAAGTCTGA |
| | AAC | AC |
| | [SEQ ID NO: 40] | [SEQ ID NO: 603] |
| XD-14786 | UACGCGGUGAAUUCUGUCU | TACGCGGTGAATTCTGTCTC |
| | CCC | CC |
| | [SEQ ID NO: 100] | [SEQ ID NO: 605] |
| XD-14790 | UUCGGGUUGAAAUCUGAAG | TTCGGGTTGAAATCTGAAGT |
| | UGU | GT |
| | [SEQ ID NO: 108] | [SEQ ID NO: 607] |
| XD-14792 | AUUAACUACUCUUUGGUCU | ATTAACTACTCTTTGGTCTG |
| | GAA | AA |
| | [SEQ ID NO: 112] | [SEQ ID NO: 608] |
| XD-14800 | UUGAUUUCGAGGAUGUCGC | TTGATTTCGAGGATGTCGCT |
| | UGG | GG |
| | [SEQ ID NO: 128] | [SEQ ID NO: 611] |
| XD-14819 | UUCGAUGCAGGACUAGCAG | TTCGATGCAGGACTAGCAGG |
| | GCG | CG |
| | [SEQ ID NO: 166] | [SEQ ID NO: 612] |
| XD-14835 | UUAGUUGAUCCAUAGAUUC | TTAGTTGATCCATAGATTCA |
| | AGA | GA |
| | [SEQ ID NO: 198] | [SEQ ID NO: 613] |
| XD-14857 | AGGAACGUGGGUUGAACUC | AGGAACGTGGGTTGAACTCC |
| | CUU | TT |
| | [SEQ ID NO: 242] | [SEQ ID NO: 615] |
| XD-14890 | UAUUGCGUGGAGUAAGCUG | TATTGCGTGGAGTAAGCTGG |
| | GUG | TG |
| | [SEQ ID NO: 308] | [SEQ ID NO: 618] |
| XD-14904 | AGCGUUAGGGUGCGCAUAC | AGCGTTAGGGTGCGCATACT |
| | UGC | GC |
| | [SEQ ID NO: 336] | [SEQ ID NO: 620] |
| XD-14917 | UGUAUACGCCGGCUGAACG | TGTATACGCCGGCTGAACGT |
| | UGA | GA |
| | [SEQ ID NO: 362] | [SEQ ID NO: 621] |

Embedding of miRNAs in AAV Plasmids

miRNA sequences such as the above are envisioned to have a therapeutic benefit for patients with neurodegenerative disease when expressed from an AAV genome. Therefore, miRNA sequences were inserted into AAV cis-plasmids, flanked by AAV2 inverted terminal repeats (ITRs). miRNAs were inserted in an intron, then followed by an exon expressing green fluorescent protein (GFP). After a stop codon, a SV40 poly adenylation sequence was inserted to ensure robust polyadenylation. The miRNA-encoding transcript was inserted downstream of either a CAG or human Synapsin promoter, as Polymerase-II promoters. The sequence was also inserted into a vector downstream of an H1 promoter, with a CBh promoter controlling the expression of GFP downstream of the H1 miRNA insert. Sequences scAAV.Syn.miR1-1.XD14792.GFP.SV40 (SEQ ID NO:622), scAAV.Syn.miR1-1.XD-14887.GFP.SV40 (SEQ ID NO:623), ssAAV.CAG.miR1-1.XD-14792.GFP.SV40pA (SEQ ID NO:624), ssAAV.CAG.miR-1-1.XD-14887.GFP.SV40pA (SEQ ID NO:625) show representative cis-plasmids with miRNA XD-14792 or XD-147887 inserted. For clinical constructs, GFP sequence are replaced by inert sequence, derived from portions of the genome expected to have no effect if expressed. For Synapsin or H1-promoter containing vectors, the insert was flanked by one full-length ITR and one ITR with a truncated terminal resolution site.

AAV plasmids were generated by conventional large-scale DNA preparation and the integrity of ITRs verified by digestion with the restriction endonuclease SmaI, with the expected banding pattern observed. Plasmids were used to package genomes containing the miRNAs into AAV9-capsid encapsidated viruses (Vector Biolabs). AAVs were titered by qPCR with primers against GFP to calculate genome counts per mL.

### AAV Tail Vein Injection

Guide sequences of XD-14792 (SEQ ID NO:112) and XD-14887 (SEQ ID NO:302) are complementary to the mouse ATXN2 transcript, with one base pair mismatch at base 22 of XD-14792. Wu et al. (PLoS One (2011) 6:e28580) and Ohnishi et al. (Biochem Biophys Res Commun (2005) 329:516-21) suggest that these 3′ mismatches do not impair knockdown.

In order to assess the ability of these viruses to knockdown ATXN2 in vivo, concentrated AAV was diluted to a concentration of 3*10¹¹ genome counts per 200 microliters in 0.001% Pluronic F-68 (Gibco #24040-032) in PBS (VWR #K812-500ML). 2 month old C57Bl/6 male mice were each injected intravenously (IV) into the tail vein with 200 microliters of virus (3*10¹¹ GC total injected for viruses with CAG promoters; 2*10¹¹ GC injected for viruses with Synapsin promoters). Fifteen days after injection, mouse tissue was processed for analysis. Following carbon dioxide-induced euthanasia and transcardial perfusion with PBS, tissues were immediately snap-frozen in liquid nitrogen. Samples were subsequently stored at −80° C.

### Western Analysis of ATXN2 Levels:

Protein extraction was performed by cutting approximately 50 mg of right medial liver tissue samples on dry ice, placing each into 500 microliters RIPA buffer (TEKNOVA #50-843-016) supplemented with protease and phosphatase inhibitor tablet (Pierce #A32959), Halt protease inhibitor cocktail (Thermo #1861279) and PMSF (Cell Signaling Technology #8553S). Tissues were disrupted in a Precellys Evolution Homogenizer (tubes=0.5 mL CK14, protocol=3×45 s 5000 rpm, 15 s break, 0° C.). Samples were incubated on ice for 30 min, centrifuged for 15 min at 17,000×g at 4° C., and supernatant was transferred to a fresh tube and stored at −80° C. Protein lysates were quantitated (Pierce, 23225), resulting in approximately 8 μg/μl protein per sample.

The NuPage system (Thermo) was used for gel electrophoresis. 20 μg of each sample was loaded onto 4-12% Bis-Tris protein gels (Thermo, NP0321BOX) and run at constant 200V for 1 hr. Revert 700 (Licor, 926-11010) was used to assay for protein loading. Proteins were transferred onto PVDF membrane (EMD Millipore, IPFL00005) overnight at 4° C. using constant 30V and 90 mA. Membranes were blocked for 1 hr at RT (Rockland, MB-070). Primary antibody incubation was performed overnight rocking at 4° C., including anti-Atxn2 (1:1000, BD, 611378), anti-GFP (1:2000, CST, 2956) and beta-actin (1:2000, CST, 4970). Washing was performed 4×5 min with TBS+0.1% tween-20, and secondary antibodies were incubated for 1 hr rocking at RT (1:15,000 each of 800CW goat anti-mouse and 680RD donkey anti-rabbit, Licor). Membranes were washed again and imaging was performed on an Odyssey Fc Imaging system (Licor). Signal quantitation was by Licor image-studio lite.

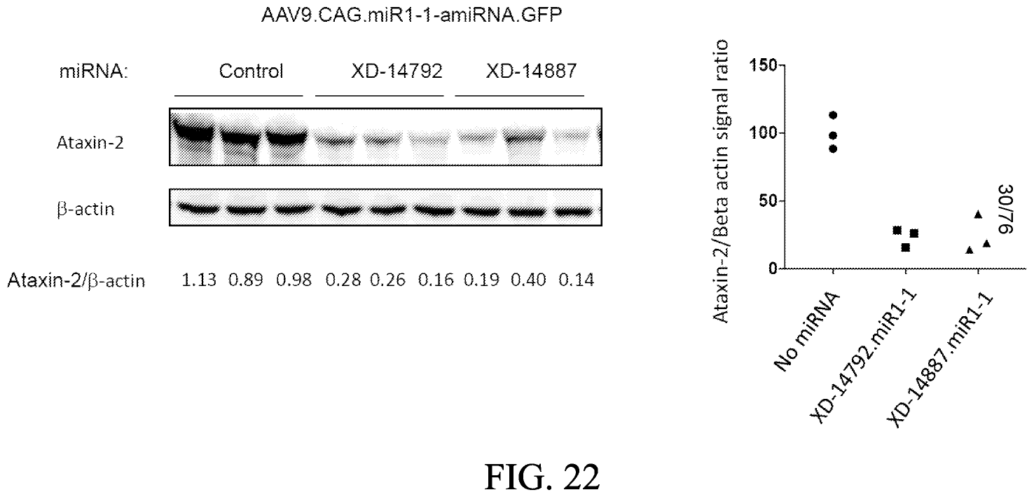

FIG. 22 (left panel) shows Western analysis of tissues from animals dosed with CAG-promoter containing viruses. Liver tissue from animals dosed with viruses expressing miRNA XD-14792 miR1-1 (SEQ ID NO:1133) or XD-14887 miR1-1 (SEQ ID NO:1149) showed a substantial reduction in ATXN2 signal, as quantified by the ratio of ATXN2 immunoblot signal to Beta-actin signal, relative to a control virus lacking a miRNA (FIG. 22 (right panel)). As expected, since expression from the synapsin promoter is CNS-enriched, AAV with a synapsin promoter expressing the same miRNAs showed much less GFP expression, and did not reduce ATXN2 protein levels (data not shown). Therefore, AAV-mediated delivery of ATXN2 targeting miRNAs can modulate ATXN2 protein levels in vivo, consistent with the therapeutic objective.

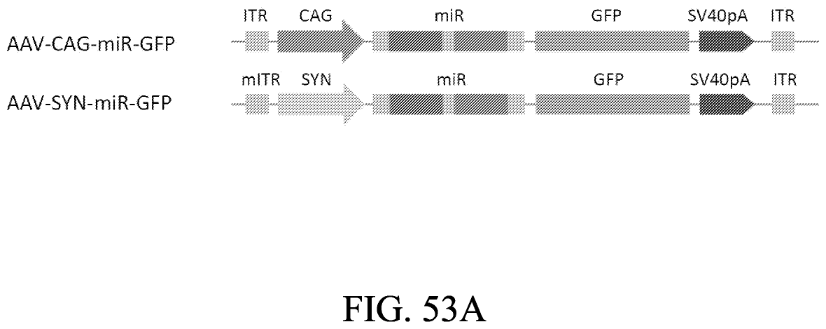

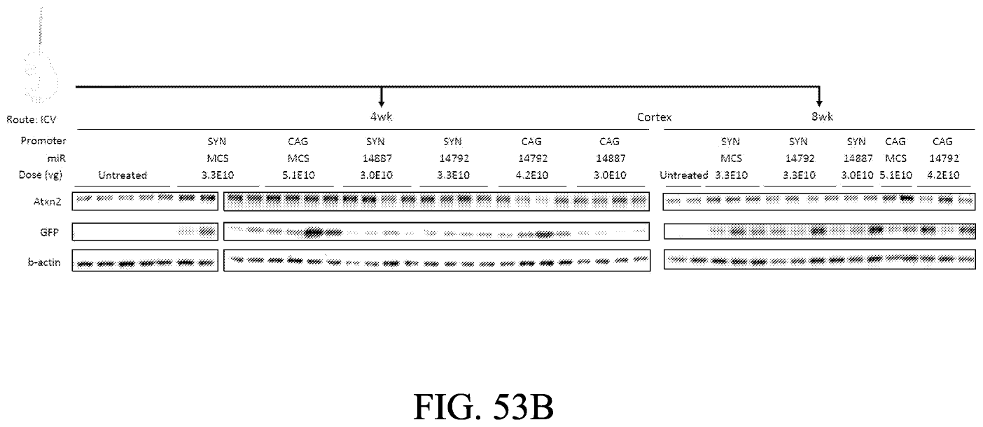

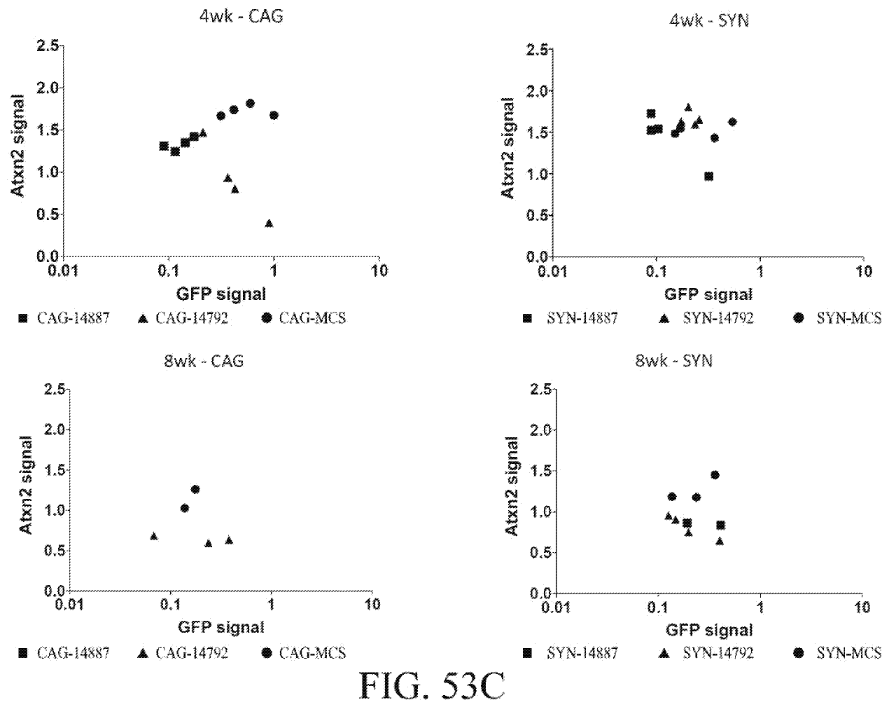

To assess whether ATXN2-targeting amiRNAs expressed from AAV dosed into the cerebrospinal fluid could lower ATXN2 levels, neonatal mice were dosed via the intracerebroventricular route (i.c.v.) at postnatal day 0 with AAV-amiRNAs with either CAG or Synapsin promoters (FIG. 53A). AAV expressing XD-14792 in miR1-1 backbone (SEQ ID NO:1133) or XD-14887 in miR1-1 backbone (SEQ ID NO:1149) were used. As in the intravenous dosing experiment, the vectors also included GFP reporters to allow for identification of transduced cells. Cortex tissue was harvested after either 4 or 8 weeks, and ATXN2 protein levels assessed by Western along with GFP levels (FIGS. 53B-53C). Decreased levels of ATXN2 protein were observed relative to tissue from animals dosed with control, non-amiRNA vectors (MCS) at both 4 and 8 weeks with CAG vectors, for XD-14792 amiRNAs, and at 8 weeks with Synapsin promoter vectors.

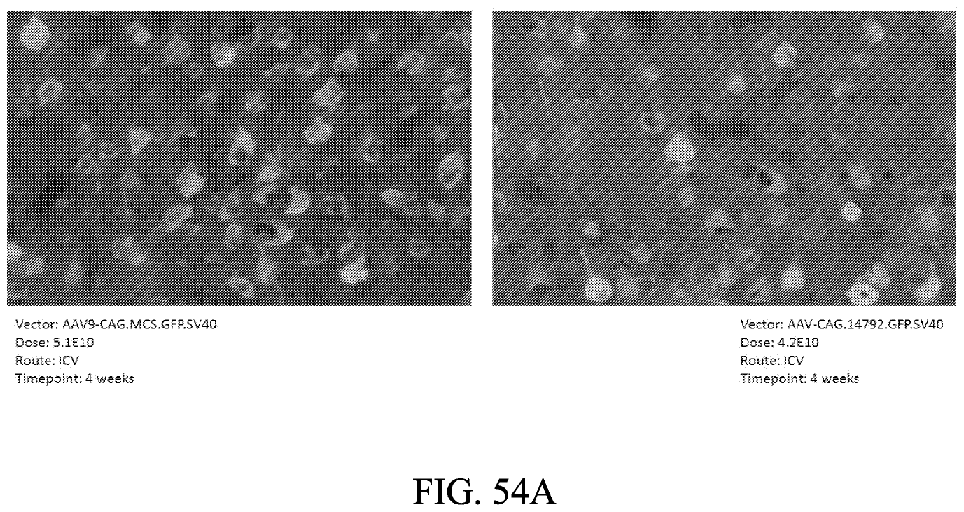

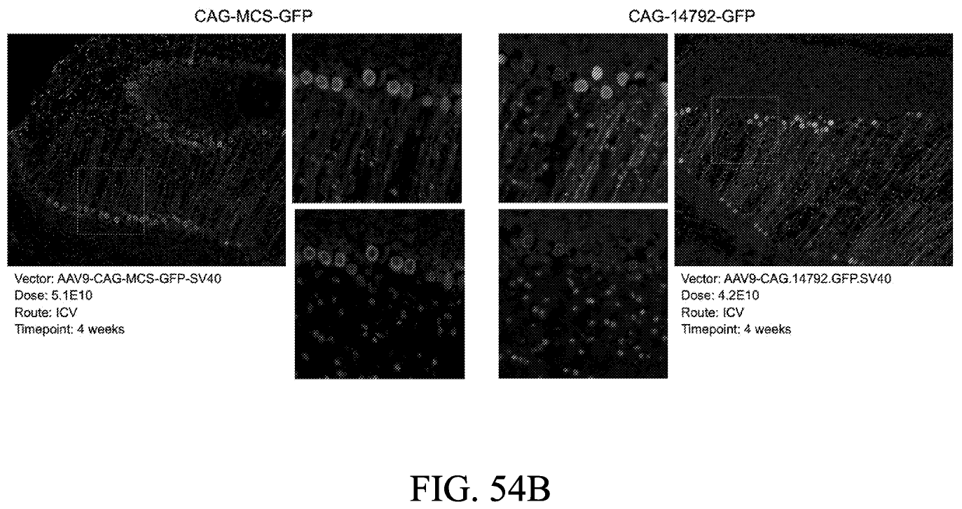

To verify that the cell types that experienced knockdown included the therapeutically intended target cell types, i.e., neurons, fixed cortex from i.c.v. dosed animals was subject to immunofluorescence analysis with antibodies against Atxn2 and GFP. Clear evidence of reduced anti-ATXN2 immunofluorescence signal was seen in the brain of animals dosed with ATXN2 amiRNAs versus animals dosed with control vector. Within individual tissue sections, transduced and untransduced cells can be distinguished by the expression of the GFP reporter. FIG. 54A shows immunofluorescence of cortex; in tissue from animals dosed with ATXN2 amiRNA (XD-14792 in miR-1-1 backbone, SEQ ID NO:1133) expressing AAVs, comparing neurons expressing GFP with neurons without GFP shows a clear reduction in Axn2 signal in GFP expressing neurons, which will also express the active amiRNA, versus neurons without the GFP. In slices from animals treated with vector without an active amiRNA, there is not an apparent difference in Atxn2 expression level between GFP expressing and non-GFP expressing neurons. Similarly, FIG. 54B shows sections of the cerebellum from animals treated with Atxn2 miRNA (XD-14792 in miR-1-1 backbone, SEQ ID NO:1133) expressing AAV or control virus. In animals treated with Atxn2 miRNA (XD-14792 in miR-1-1 backbone, SEQ ID NO: 1133), GFP expressing neurons (which will also express the Atxn2 miRNA) have lower levels of Atxn2 signal.

### Materials and Methods

ATXN2 siRNA Transfection for Immunostaining

U2OS cells (unmodified; wildtype) were seeded at 5,000 cells/well 1 day prior to siRNA transfection in 96-well Flat Clear Bottom Black Polystyrene TC-treated microplates (Corning, P/N 3094). After siRNAs were diluted from stock solutions into Opti-MEM I Reduced Serum Medium (Gibco, P/N 31985-062), transfection mixtures were generated using Lipofectamine RNAiMAX Transfection Reagent (Invitrogen P/N 56532). Transfection mixtures were then aliquoted onto U2OS cells using the Apricot S-PIPETTE S2 and placed into the tissue culture incubator at 37 C/5% CO₂/20% 0₂.

ATXN2 siRNA Immunostaining and Imaging Protocol

Three days after transfection, cells were fixed (4% paraformaldehyde/4% sucrose final), followed by washing (PBS), blocking and permeabilization (IF buffer: 0.5% BSA, 0.2% saponin, 5% goat serum). Primary antibody (BD 611378) was applied to the cells at 1:200 in IF buffer in an overnight incubation. Following PBS washing, cells were incubated in secondary antibody (Life Technologies, Alexa Fluor Plus 488) followed by a DNA stain (Hoechst 33342). After final PBS washing, cells were incubated overnight at 4 C followed by imaging the next day. Using the Thermo Scientific Invitrogen EVOS FL Auto 2 Imaging System with a 20× objective, images were captured by autofocusing on the nuclear DNA stain, capturing the DNA stain, then auto-repositioning to capture the ATXN2 signal with a total of 9 fields imaged per well.

Artificial miRNA Construct Development

Oligonucleotides (Twist) containing Atxn2 targeting shRNAs embedded within miR-1-1 and miR-155E backbones were PCR amplified using regions common to all oligonucleotides (Forward: TAAGCCTGCAGGAATTGCCTAG (SEQ ID NO:626), Reverse: CATGTCTCGACCTGGCTTACTAG (SEQ ID NO:627)). Following amplification, PCR products were verified for the correct sized product by gel electrophoresis. Diluted PCR products were then inserted into a Xba1 and EcoRI-digested pLVX EF1alpha>mCherry CMV>Puro construct, similar to SEQ ID NO:546 using NEB HiFi DNA Assembly Master Mix (NEB P/N M5520AA). A portion of the reaction mixture was then incubated with NEB Stable Competent E. coli cells (NEB P/N C3040H) on ice, heat shocked at 42° C., allowed to recovery on ice, followed by addition of S.O.C. media and incubated at 30° C. The bacterial culture was then applied to LB agar plates with the antibiotic Carbenicillin and grown overnight at 30° C. Individual bacterial colonies were sanger sequence verified (Primer: CATAGCGTAAAAGGAGCAACA (SEQ ID NO:628)). After verifying the correct insert based on the Sanger sequencing, bacterial cultures were grown and the plasmid DNA purified and quantified.

### Virus Production

With sequence-verified constructs, lentivirus was produced using Lenti-X 293T cells (Takara) and the pc-Pack2 Plasmid Mix (Cellecta P/N CPCP-K2A). Using the Lipofectamine 3000 Transfection Kit (Invitrogen, P/N L3000-008), Lenti-X 293Ts were transfected with individual pLVX EF1a>mCherry miR insert CMV>Puro constructs and the pc-Pack2 Plasmid Mix. The transfection-containing media was aspirated and replaced with viral product media (VPM; 293T media+20 mM HEPES (gibco, P/N 15630-08)). VPM was collected 48 hours later and aliquoted into 96-well 2.0 mL Deepwell plates (Thermo, P/N 4222) and frozen at −80° C.

### Viral Transduction

Prior to adding the VPM to cells, U2OS wildtype (unmodified) and ATXN2 knockout (C43) were seeded at 5,000 cells/well 8 hours prior in 96-well Flat Clear Bottom Black Polystyrene TC-treated microplates (Corning, P/N 3094). After adding polybrene (8 μg/ml final, Cellecta, P/N LTDR1), thawed VPM was added using Apricot S-PIPETTE S2. The cells were then placed into the tissue culture incubator at 37° C./5% CO₂/20% O₂. The media on the cells containing the VPM and polybrene was removed 12 hours later and replace with fresh media (U2OS media only) and placed into the tissue culture incubator at 37° C./5% CO₂/20% O₂.

ATXN2 pLVX EF1a>mCherry miR Insert CMV>Puro Immunostaining and Imaging Protocol

Three days after changing the media (3.5 days since after the VPM), cells were fixed (4% paraformaldehyde/4% sucrose final), followed by washing (PBS), blocking and permeabilization (IF buffer: 0.5% BSA, 0.2% saponin, 5% goat serum). Primary antibodies (Atxn2; BD 611378, 1:200 dilution, mCherry; ab205402, 1:1000 dilution) were applied to the cells in IF buffer in an overnight incubation. Following PBS washing, cells were incubated in secondary antibody (Life Technologies, Alexa Fluor Plus 488 and Alexa Fluor Plus 647) followed by a DNA stain (Hoechst 33342). After final PBS washing, cells were incubated overnight at 4° C. followed by imaging the next day. Using the PerkinElmer Operetta CLS High-Content Analysis System with a 20× objective, non-confocal images were captured by autofocusing the bottom of the plate, then capturing the DNA signal, the ATXN2 signal and the mCherry signal with a total of 9 fields imaged per well.

miR-155 and miR-1-1 Transfection and ATXN2 Western Blot

A version of the miR-155 scaffold was engineered into an artificial miRNA and used in a mouse in vivo proof of concept study to knockdown HTT¹⁰. ATXN2-targeting guide sequences and controls were incorporated into this scaffold sequence, which we term “miR-155M,” and assayed for protein knockdown after transfection of U2OS cells.

The “miR-1-1E,” where “E” signifies “enhanced,” is taking the human miR-1-1 scaffold and simply introducing a downstream CNNC motif.

To perform the transfection, U2OS cells were plated at 90,000 cells/well in a 12-well dish, 24 hours later, transfected 2 micrograms/well of the 8 EF1alpha>mCherry constructs (7 with inserts, 1 control) with Lipofectamine 3000 (ThermoFisher). Specifically, each transfection used 2 μL enhancer reagent, 1.5 μL lipofectamine reagent; diluted samples in water to uniform amounts).

Following day imaging with Evos, a good number of mCherry cells observed. Much higher expression level observed in the control vector without insert.

Wells were aspirated and replaced with 1 ml of media with 1 microgram/mL puromycin. Selection occurred over the weekend and then puromycin was removed for recovery.

Three days post-selection, many dead cells were observed. Imaging of mCherry indicated there remained a good number of bright, surviving cells, however. Aspirated media and replaced with prewarmed media containing 200 ng/mL puromycin (a 5-fold dilution).

Two days later (7 days post-transfection), cells were lysed in RIPA buffer with Pierce phosphatase and protease inhibitor tablet.

Western blot was performed and imaged on Odyssey Fc (Licor).

Quantitation of ATXN2 band and control α-tubulin signal intensity was performed with ImageStudio software (LiCor).

### Generation of ATXN2 Knockout in U2OS Cells

ATXN2 knockout cells in U2OS cells was performed using a Cas9-gRNA RNP nucleofection approach. In brief, crRNA and tracrRNA (IDT) were duplexed at equimolar ratios and complexed with recombinant Cas9 (IDT v3) and nucleofected using SF buffer and CM130 program (Lonza 4D nucleofector).

CRISPR guide RNAs were selected from two CRISPR library sources. Three CRISPR guide RNAs (gATXN2_1, gATXN2_2, gATXN2_3) were chosen from the Cellecta CRISPR cutting library (one was not selected due to its upstream position before the 2^{nd} ATG). Two additional guides (gATXN2_4 & gATXN2_5) were chosen from the another CRISPR cutting library reported by Bassik et al.²⁶. Additionally, a non-targeting control guide was chosen from the Cellecta library. CRISPR guide RNA sequences as well as DNA format are provided in Table 14.

TABLE 14
CRISPR Guide RNA Sequences for Targeting ATXN2

| Name | Guide DNA Sequence | Guide RNA Sequence |
|---|---|---|
| gATXN2_1 | AAGTTACTCACCGTAGACTG | AAGUUACUCACCGUAGACUG |
| | [SEQ ID NO: 629] | [SEQ ID NO: 1169] |
| gATXN2_2 | AATAGAGAAGTCATATCCTG | AAUAGAGAAGUCAUAUCCUG |
| | [SEQ ID NO: 630] | [SEQ ID NO: 1170] |
| gATXN2_3 | GTATTGGAAATACCCCCAGT | GUAUUGGAAAUACCCCCAGU |
| | [SEQ ID NO: 631] | [SEQ ID NO: 1171] |
| gNTC | GTAGCGAACGTGTCCGGCGT | GUAGCGAACGUGUCCGGCGU |
| | [SEQ ID NO: 632] | [SEQ ID NO: 1172] |
| gATXN2_4 | GTGAGTTCACCTGCATCCCA | GUGAGUUCACCUGCAUCCCA |
| | [SEQ ID NO: 633] | [SEQ ID NO: 1173] |
| gATXN2_5 | GCTATCAGTGCTAAAGTGAA | GCUAUCAGUGCUAAAGUGAA |
| | [SEQ ID NO: 634] | [SEQ ID NO: 1174] |
| gCD81 | GTTGGCTTCCTGGGCTGCTA | GUUGGCUUCCUGGGCUGCUA |
| (cutting | [SEQ ID NO: 635] | [SEQ ID NO: 1175] |
| control that | | |
| cuts the CD81 | | |
| gene) | | |

Post nucleofection, bulk population of cells were allowed to recover for 5 days and lysed for western blot analysis.

### U2OS Clone Selection

The bulk population of cells were also single cell sorted into 96-well plates for clonal expansion. Because guides gATXN2_1 and gATXN2_5 had the most decrease in ATXN2 protein signal by western blot (˜90% reduction), we proceeded with these cells for single cell cloning. After trypsinization and single cell suspension, a SONY SH800S was used to gate for singlet cells and to sort directly into U2OS growth media. Cells were allowed to grow for ˜2-3 weeks and lysed for genomic DNA extraction for Sanger sequencing and protein extraction for western blotting (10 μg of protein used per lane in this setting)

### ICE Confirmation of Clones

Genomic DNA was extracted using a Qiagen Blood and Tissue Kit. Genomic primers were designed to amplify the genomic region surrounding the guide RNA cut site with the goal of sequencing the cut site by Sanger sequencing and validating an out-of-frame indel pattern consistent with a single clone.

Primer Blast (https://www.ncbi.nlm.nih.gov/tools/primer-blast/) was used with the following settings: For guide 1, we turned off repeat filter and low complexity filter due to the repetitive nature of ATXN2, but otherwise kept the default settings. The import function of Snapgene was used to import “6311” from NCBI. 500 upstream and 500 downstream bases from the protospacer sequence was used to as input for primer blast. Product size was set for 400-1000 and 2 distinct primer pairs were selected (Table 15).

TABLE 15
ATXN2 PCR primers

| Primer Name | Primer Sequence |
|---|---|
| Forward primer | AAGTTTCCTGAGGCCTCCCC [SEQ ID |
| (ICE_gATXN2_1_set1_Fwd) | NO: 636] |
| Reverse primer | TCCAGGCAAGCAGTGCATAG [SEQ ID |
| (ICE_gATXN2_1_set1_Rev) | NO: 637] |
| Product length 522 | |
| Forward primer | AGTTTCCTGAGGCCTCCCC [SEQ ID NO: 638] |
| (ICE_gATXN2_1_set2_Fwd) | |
| Reverse primer | CCCTACCTGTTGTGGGTCTC [SEQ ID |
| (ICE_gATXN2_1_set2_Rev) | NO: 639] |
| Product length 539 | |

Furthermore, amplicon internal sequencing primers were designed for Sanger sequencing in both forward and reverse directions to read the cut site (Table 16). The primer(+) algorithm (http://www.biology.wustl.edu/gcg/prime.html) was used to design the sequencing primers on this web interface (https://www.eurofinsgenomics.eu/en/ecom/tools/sequencing-primer-design/).

TABLE 16
ATXN2 Sequencing Primers

| Primer Name | Primer Sequence |
|---|---|
| ICE_gATXN2_1_Rseq | AGAGCCATCCTTAGGTAGCC [SEQ ID NO: 640] |
| ICE_gATXN2_1_Fseq | GAGGTTTAATTGACTCATGCTCTG [SEQ ID |
| | NO: 641] |

For guide 5, we turned off repeat filter but turned on the low complexity filter, but otherwise kept the default settings. 500 upstream and 500 downstream bases from the protospacer sequence was used to as input for primer blast. Product size was set for 400-1000 and 2 distinct primer pairs were selected (Table 17).

TABLE 17
ATXN2 Primer Sequences

| Primer Name | Primer Sequence |
|---|---|
| Forward primer | AAAACACACCGGCATTTCCC [SEQ ID NO: 642] |
| (ICE_gATXN2_5_set1_Fwd) | |
| Reverse primer | CCTGGGCAACAGAACGAGAC [SEQ ID |
| (ICE_gATXN2_5_set1_Rev) | NO: 643] |
| Product length 853 | |
| Forward primer | AGCAAAACACACCGGCATTT [SEQ ID |
| (ICE_gATXN2_5_set2_Fwd) | NO: 644] |
| Reverse primer | ATCACGCCACTGCATTCCA [SEQ ID NO: 645] |
| (ICE_gATXN2_5_set2_Rev) | |
| Product length | |

Internal sequencing primers were designed by the primer(+) algorithm (Table 18).

TABLE 18
ATXN2 Sequencing Primers

| Primer Name | Primer Sequence |
|---|---|
| ICE_gATXN2_5_Fseq | TCATGAGCATCCACAAGAACAG [SEQ ID NO: 646] |
| ICE_gATXN2_5_Rseq | ACGTGTGTGAGTGTAACTGATTGC [SEQ ID |
| | NO: 647] |

PCR was performed with NEBNext Ultra II Q5 Master Mix (NEB, M0544S) with gDNA and primer pairs indicated above. Amplified products were visualized by agarose gel and correctly sized amplicons were gel purified and submitted for Sanger sequencing with forward and reverse sequencing primers. Chromatogram (.abi files) results were uploaded to the Inference of CRISPR Editing (ICE) tool²⁷ https://ice.synthego.com/#/ for deconvolution of Sanger reads to identify indels.

Clone 43 from guide 5 nucleofection, which verified both by western and Sanger sequencing as a bona fide knock-out clone, was carried forth for further studies.

### Ataxin-2 Western Blot

To prepare lysates, 1×RIPA (Teknova, Tris-HCl 50 mM, NaCl 150 mM, 1% Triton X-100, Sodium Deoxycholate 1%, SDS 0.1%, EDTA 2 mM, pH 7.5) was supplemented with Pierce protease/phosphatase tablet (Thermo, A32959) and incubated 15 min on ice, spun down at 17,000×g at 4° C. for 15 min.

Pierce BCA kit (Thermo Scientific, 23225) was used for protein quantitation and 20 μg of protein was loaded per lane in SDS-PAGE gel electrophoresis (NuPage Bis Tris, Thermo Scientific).

Samples were prepared with 10 or 20 micrograms of protein, 4×LDS loading buffer (NP0007), 10× sample reducing agent (NP0004), water to 20 μl. Samples were heated at 70° C. for 10 min.

Protein size ladders were Precision plus protein dual color standard (Bio-rad, 1610374) or Chameleon® Duo Pre-stained Protein Ladder (Licor, 928-60000).

Samples were loaded onto 1.0 mm×10 well 4-12% Bis-Tris protein gel (NP0301PK2) and gel electrophoresis was run with MOPS SDS running buffer (NP0001) for 1 hr 20 min at constant 200V to resolve higher molecular weight bands.

Tris/glycine transfer buffer was used (Bio-rad, 1610734) without methanol. All components including sponges, filter paper, gel, and membrane were equilibrated at least 15 min with transfer buffer. The PVDF membrane was dipped in methanol for 15 seconds prior to equilibration with transfer buffer. Wet transfer was performed in a Mini Trans-Blot Electrophoretic Transfer Cell (Bio-rad, 1703930) overnight at 4° C. at constant 30V, 90 mA.

After overnight transfer, membranes were air dried for 1 hr at RT. Membranes were rinsed with 1×TBS (no tween) and blocked in Odyssey blocking buffer (LI-COR) at room temperature rocking for 30 min-1 hr.

Membranes were incubated with primary antibodies overnight at 4° C. at 1:1000 dilutions in Odyssey blocking buffer with 0.1% Tween-20. The mouse ATXN2 antibody (BD, 611378) and Rabbit α-Tubulin antibody (CST, 21445) was used as a loading control.

Membranes were washed 4×5 min with TBS-0.1% Tween-20.

Membranes were treated with two secondary antibodies for 1 hr rocking at RT at 1:20,000 dilutions in Odyssey blocking buffer with 0.1% Tween-20 and 0.01% SDS.

The secondary antibodies were IRDye 800CW Goat anti-mouse IgG, (Li-cor, 926-32210) and IRDye 680RD Donkey anti-rabbit IgG (Li-cor, 926-68073). Membranes were washed 4×5 min with TBS-0.1% Tween-20 and rinsed with TBS (no Tween) before imaging on a LI-COR Odyssey scanner (Fc) with both 700 and 800 channels.

### Ataxin-2 FACS

Cells were trypsinized, transferred to a 96-well v-bottom format, each treatment assayed in triplicate, and washed in wash buffer (PBS/0.5% BSA (no EDTA)) and fixed with ice-cold methanol dropwise, incubated on ice for 10 min, then 200 μl of PBS were added and cells were rocked overnight at 4° C.

Cells were spun down at 1000×g, 5 min cold and washed twice with cold FACS wash buffer (PBS/0.5% BSA/2 mM EDTA/0.2% saponin). Primary antibody (BD 611378) was applied at 1:100 and incubated for 1 hr, rocking in 4° C. The buffer was supplemented with 5% goat serum to reduce non-specific binding. Cells were washed twice in cold FACS wash buffer. Cells were incubated in 1:100 secondary antibody (PE/Cy7 Biolegend clone RMG1-1) with cells resuspended in cold FACS wash buffer with 5% goat serum and incubated for 1 hr on ice. Cells were washed twice and resuspended in cold FACS wash buffer and sampled on an Attune (Thermo Scientific).

### Intracerebroventricular Injections

For intracerebroventricular injections, postnatal day 0 pups were cryo-anesthetized and injected at a depth of 2 mm using Hamilton syringes, delivering a maximum volume of 3 uL per each ventricle.

### Immunofluorescence Analysis

Animals dosed i.c.v with rAAV were euthanized 4 weeks after dosing with rAAV, fixed overnight in 4% paraformaldehyde. Tissue was then cryopreserved in cold 30% sucrose, then embedded in OCT media and frozen. 5 micrometer frozen sections were prepared on a cryostat. For staining, sections were thawed and dried, washed twice in PBS, heated in 95 C antigen retrieval solution (citra antigen retrieval, pH 6.0, Vector Labs #H-3300-250) for 10 minutes, then cooled for 30 minutes at room temperature. Sections were then washed 5 minutes each in water, PBS, and PBS-0.25% Triton-X-100, and 10 minutes in PBS. Sections were then blocked with 5% goat serum in PBS for 30 minutes in humidified chambers. Sections were treated with primary antibody solution in PBS+1% BSA, including: Mouse anti-ATXN2 antibody (BD #611378), 1:50; Rabbit anti-GFP antibody (Cell Signaling Technologies #25555), 1:2000 overnight at 4 C. After 3× washes in PBS, sections were incubated with secondary antibody solutions in PBS+1% BSA, including: goat anti-mouse Alexa Fluor 555 (Thermo Scientific #A21424) 1:250, Goat anti-Rabbit Alexa Fluor 488 (Thermo Scientific #A11008), 1:250 for 30 minutes at room temperature. Sections were then washed, and mounted in VectaShield PLUS with Dapi (H-2000-10). Images were collected with a Revolve microscope (Discover Echo).

### Example 2: Identification of High Performing AmiRNAs by Tiled Screen of ATXN2 Targeting miRNAs in Lentiviral Format

As an alternative approach to siRNA screening followed by embedding of the associated guide sequences in miRNA backbones and testing one-by-one, pooled screening of ATXN2-targeting miRNAs was conducted (“Deep Screen 1”).

### ATXN2 Target Sequences

Homo sapiens ATXN2 mRNA (NM_002973, transcript variant 1, SEQ ID NO:2) was used to identify target sequences for the artificial miRNAs. All human and primate cross-reactive sequences were identified and 22-nt guide sequences were designed taking into consideration criteria for effective shRNA and miRNA sequences, including the preference for A or U at guide position 1. Therefore, taking into consideration the 22 nucleotide antisense sequences complementary to the Ataxin-2 construct, if the first guide base was G or C this was converted to a ‘U’, whereas sequences that began with A or U were not changed from the base complementary to the corresponding position on the ATXN2 transcript. As above, U bases are encoded as T in the lentiviral expression construct. In total 2,381 ATXN2-targeting sequences were introduced into a modified variant of the miR-16-2 backbone. Passenger sequences (the sequence on the opposite side of the miRNA stem from the guide sequence) were generated following the rules in Table 8 for this backbone.

### Toxicity Controls

By examining the abundance of elements of the library in cells that had been allowed to grow for lengthy periods of time versus initially transduced cells, the pooled screen can identify elements that alter cellular proliferation or viability. To calibrate the dynamic range of the assay, additional toxic elements were added to the library. Ten essential genes were selected with ten shRNAs each (removing 2 sequences that had polyT sequences deemed problematic because they may serve as termination signals for PolIII). To identify the “essential” gene list, genetic dropout screens performed in parallel with shRNA and CRISPR guide RNAs in the K562 cancer cell line²¹ were examined. Across both screens, genes were rank ordered by shRNA lethality, specifically genes that scored highly in the K562 shRNA dropout by combined Castle score (negative is more lethal). Since toxicity screen was performed in Hela cells, the K562 top genes were intersected and identified the top 10 genes that also scored highly (bayesian factor >100) in a Hela CRISPR cutting dropout screen²². The essential genes selected were: COPB1, COPB2, DHX15, EIF3A, EIF4A3, NUP93, PRPF8, PSMB6, PSMD1, and SF3B2.

To select 10 shRNA targeting each gene, the 25 shRNA/gene in a previously published shRNA library were considered and rank ordered by their performance in the dropout screen¹⁵. Specifically, the shRNAs were rank-ordered by the dropout metric (read counts in replicate 1 and replicate 2 divided by plasmid reads), and the top performing shRNAs that had at least one count across all replicates were selected.

### GFP Controls

GFP controls (n=50) were designed to target two different GFP reporter systems. The first system involved tagging endogenous ATXN2 with the 11^{th} beta strand of GFP (GFP₁₁) in conjunction with overexpression of GFP₁₋₁₀ to constitute a self-complementary GFP system²³, and the second is a GFP-stop-ATXN2 overexpression reporter. The 11^{th} beta strand of GFP was targeted by entirely tiling the transcript with 28 individual 21 nt shRNA, adding an A at guide position 1 to form 22 nt oligomer sequences. Additional shGFP (n=22) were selected to target GFP₁₋₁₀ using the Design portal of the Broad Institute Genetic Perturbation Platform (https://portals.broadinstitute.org/gpp/public/seq/search), using the GFP₁₋₁₀ sequence as input. Although the split GFP system was not ultimately used to read out ATXN2 levels, the 50 shGFP still target the GFP-stop-ATXN2 reporter.

### ATXN2 Transcript Scrambled Controls

Neutral controls were designed that should not have any effect in both the efficacy and toxicity screens. These elements can be used for baseline normalization. The guide sequences targeting ATXN2 were scrambled and 974 of these scrambled guide sequences used to construct amiRNAs as before. After scrambling, the same rules for the first base as with targeting sequence were imposed. Following this correction step, the GC content was adjusted by converting one of the guide bases 2-22 that were A or T, randomly selected, to G or C, randomly chosen, such that overall this set of scrambled controls maintains similar GC content relative to the ATXN2-targeting sequences.

### Promoter Selection

The H1 promoter, an RNA polymerase III promoter, was selected to drive artificial miRNA expression as many groups have used it to achieve robust target knockdown.

### Pooled Library Cloning

The oligonucleotide pool was synthesized on chip (oligo length 172 bp, Agilent), PCR amplified, and cloned into the pRSICPH1 vector (Cellecta) by Bpl1 restriction digestion and T4 ligase ligation. Each individual miRNA cassette was expressed under the control of an H1 promoter and subsequently followed by a short constant region and 17 bp barcode sequence that uniquely tags each miRNA. The elements were designed to contain both miRNA and barcode tags to enable multiple ways to amplify and sequence the constructs to readout the pooled screens. For instance, if PCR amplification bias were to confound the representation of high GC content sequences²⁴, comparison of the abundance of amplicons containing the guide sequence versus the abundance of amplicons containing the FREE barcode would resolve any discrepancies. FREE barcodes were used as they are indel-correcting and robust to DNA synthesis and NGS errors²⁵. The library was checked by Sanger sequencing and next-generation sequence (Illumina) to verify lack of synthesis errors, >99% amiRNA and FREE barcode were correctly paired, and the fold-representation between the top and bottom amiRNAs were within four fold-change.

### Viral Production

Lenti-X 293T (Takara) cells were used to produce lentivirus by transfection of 4^{th} generation packaging plasmids (Lenti-X Packaging single shots, Takara) followed by viral concentration with Lenti-X concentrator and resuspension in PBS. Virus was titered in U2OS and Hela cells by infection and antibiotic selection followed with estimation of viral units and multiplicity of infection (MOI) by Cell-Titer-Glo (Promega).

### Cell Culture and Transfections

U2OS cells and the GFP-ATXN2 reporter cell line were cultured in RPMI-1640 supplemented with 10% fetal bovine serum (FBS) and penicillin/streptomycin/glutamine. Hela cells were cultured in DMEM supplemented with 10% fetal bovine serum (FBS) and penicillin/streptomycin/glutamine.

### Efficacy Screen

Two pooled lentiviral miRNA screens for on-target efficacy were performed to identify miRNA that diminish ATXN2 protein signal, reading out ATXN2 levels by 1) an exogenous GFP-stop-ATXN2 reporter or 2) endogenous ataxin-2 antibody in a FACS assay. Cells were infected with the pooled lentiviral library at a multiplicity of infection (MOI) of 0.1 into (˜5×10⁷ cells) with polybrene (8 μg/ml, EMD Millipore) and distributed across four T225 flasks. Two days post-infection, U2OS cells were selected with puromycin at 2 μg/ml. The MOI was confirmed by cell-titer-glo at day 5 (3 days after selection) in a 96 well format. An unsorted fraction (7×10⁶ cells) was collected at day 7 as a reference control. The remaining cells were washed in wash buffer (PBS/0.5% BSA (no EDTA)) and fixed with ice-cold methanol dropwise while vortexing on day 7, at a ratio of 1 ml methanol/2×10⁶ cells, incubated on ice for 10 min, then 10× volumes of PBS were added and cells were rocked overnight at 4° C.

Cells were spun down at 1000×g, 5 min cold (Corning 500 ml centrifuge tubes, 431123) and resuspended in cold FACS wash buffer (PBS/0.5% BSA/2 mM EDTA/0.2% saponin). Cells were counted and resuspended in 2×10⁶/ml in cold FACS wash buffer.

Primary antibody (BD 611378) was applied at 1:200 and incubated for 1 hr, rocking in 4° C. The buffer was supplemented with 5% goat serum to reduce non-specific binding.

Cells were washed twice in cold FACS wash buffer. Cells were incubated in 1:200 secondary antibody (PE/Cy7 Biolegend clone RMG1-1) with cells resuspend in 2×10⁶/ml cold FACS wash buffer with 5% goat serum and incubated for 1 hr on ice. Cells were washed twice in and resuspended in cold FACS wash buffer at 4-5×10⁶/ml to achieve 1000-2000 events per second on the Sony SH800S (approximately the maximal stable cell velocity on the instrument). Samples were filtered through a cell strainer directly into FACS tubes (FALCON 352235). Sorted cells were collected in 3 mL PBS/10% FBS in 15 ml conicals.

### Dropout Screen

A pooled lentiviral miRNA screen for off-target toxicity was additionally performed, by identifying miRNA dropout between an early and late timepoint. HeLa cells were infected with polybrene (8 μg/ml, EMD Millipore) at a multiplicity of infection of 0.1 at 1000× representation (that is, the number of cells was >10,000× the number of library elements). Two days post-infection, HeLa cells were selected with puromycin at 0.5 micrograms/mL. Cells were passaged for a total of 10 doublings (˜16 days). The screen was performed in triplicate (3 separate infections).

### DNA Processing

Genomic DNA was extracted from each sample using the Machery Nagel Blood L kit (FACS collections; early and late collection timepoints). A two-step PCR was conducted. In a first PCR reaction, an amplicon spanning both the guide and passenger sequences, and downstream past the FREE barcode, was generated. In a second PCR reaction, a nested amplicon was generated spanning either the guide and passenger sequence, or the FREE barcode. The second PCR was designed to incorporate Illumina binding sequences (P5 and P7) and sample index barcodes to enable demultiplexing on Illumina sequencing platforms. Each distinct sample (that is, FACS collection, or timepoint) was given a distinct index. Specifically, the guide and passenger amplicon was single-indexed, with an i7 sequence included upstream of the 6 nt sample barcode and P7 sequence. In contrast, the FREE barcode amplicon was single-indexed on the P5 end and no i7 sequence was included on the P7 end. Samples were sequenced on an Illumina MiSeq such that guide and passenger sequences can be matched in paired reads, with read 1 using a custom primer reading the 22 nt guide sequence, and read 2 being the standard Illumina primer reading the passenger sequence. FREE barcodes were also separately amplified and sequenced, with read 1 being a custom primer reading the 17 nt FREE barcode, and read 2 being a custom primer reading the 6 nt sample barcode. In general, calculations of abundances were highly similar for FREE barcode derived amplicons and guide/passenger sequence containing amplicons (using a lookup table of the association between FREE barcodes and guide/passenger sequences). Analyses below focused on counts of the guide sequences.

### Computational Analysis

Occurrences of each guide sequence were counted, without tolerating sequencing or other errors (that is, no mismatches to the library input guide sequences were tolerated), in read 1 sequences, which directly sequences amiRNA guide sequences. To estimate ATXN2 knockdown efficiency, the abundance of guide sequence counts in the ATXN2 high FACS collection was divided by the abundance of guide sequence counts in the ATXN2 low FACS collection. Sequences that effectively knock down ATXN2 are enriched in the ATXN2 low FACS collection.

To assess whether the guide sequence influences cytotoxicity or reduces proliferation, the ratio of counts of each guide sequence for a pool of cells collected 16 days after library transduction, versus the ratio of counts for the library collected 18 hours after library transduction, were measured.

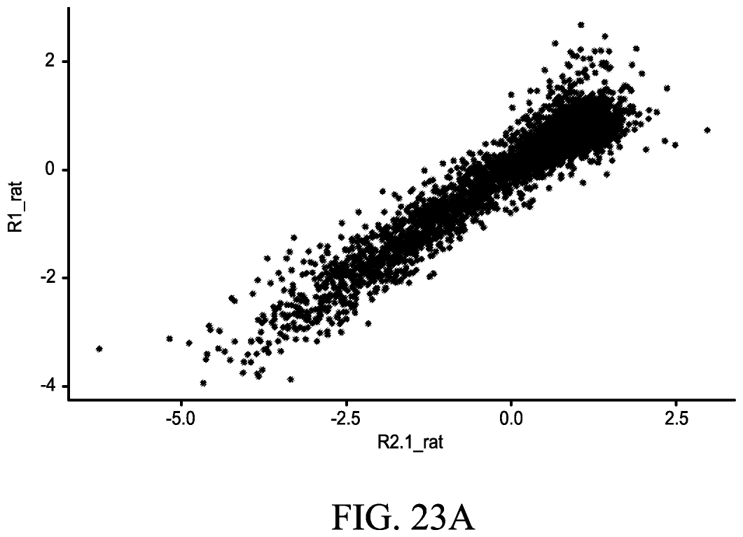

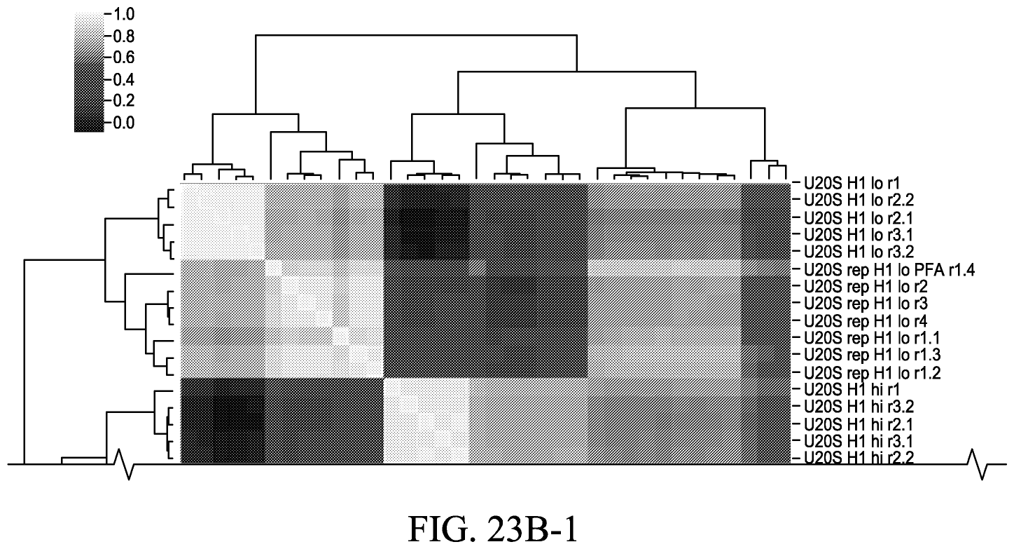

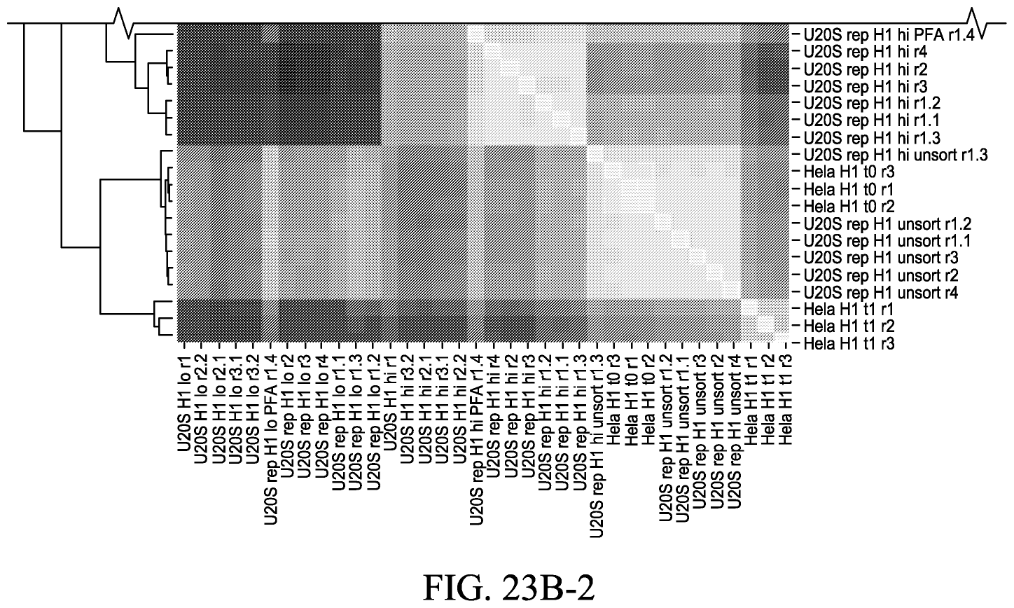

Data was highly consistent across replicates. FIG. 23A plots the high/low count ratios for two independent replicates against one another. Most points fall along y=x, indicating good correlation. FIG. 23B plots the matrix of Spearman correlation coefficients for count values for each condition against all others. The replicates are hierarchically clustered, and clustered blocks represent similar conditions. Note the strong anticorrelation between low and high conditions, as expected given that guides that deplete ATXN2 are expected to be differentially present in the low and high conditions. Note also that conditions where ATXN2 signal was visualized by antibody staining against endogenous Ataxin-2 protein, and conditions where the signal was visualized by fluorescence of the ATXN2 GFP reporter, correlate.

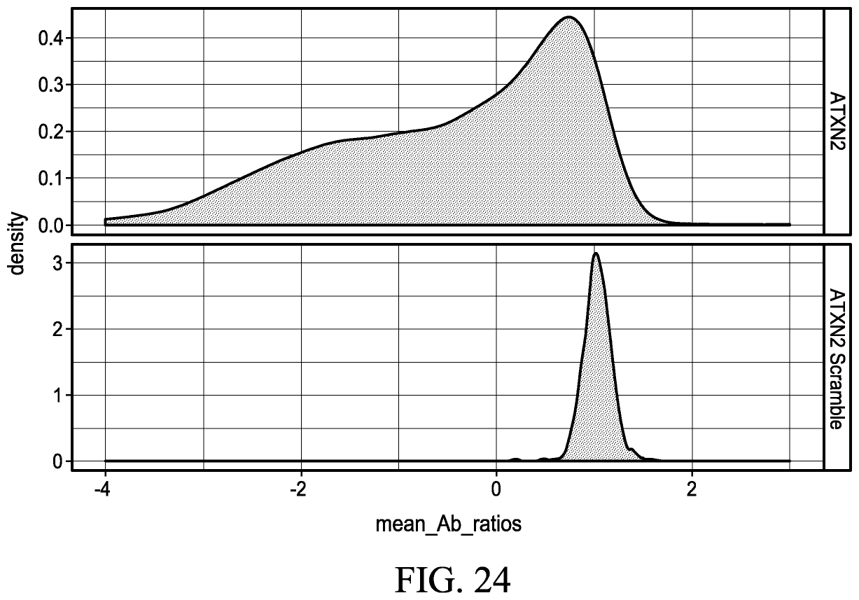

Following the calculation of count ratios, a normalization procedure was taken to rank ATXN2 targeting sequences by their ability to deplete ATXN2 signal. In FIG. 24, histograms for the distribution of high and low condition guide sequence counts for ATXN2 targeting guides, top trellis, and scrambled sequences, bottom trellis, are shown. The ATXN2 scrambled sequences exhibit a sharp, unimodal distribution of ratios of counts in the high and low ATXN2 FACS conditions. The median ratio from this distribution was taken to be no-effect, and the ATXN2 depleting effect of ATXN2 targeting miRNAs was therefore calculated by subtracting this (log base 2-transformed) value.

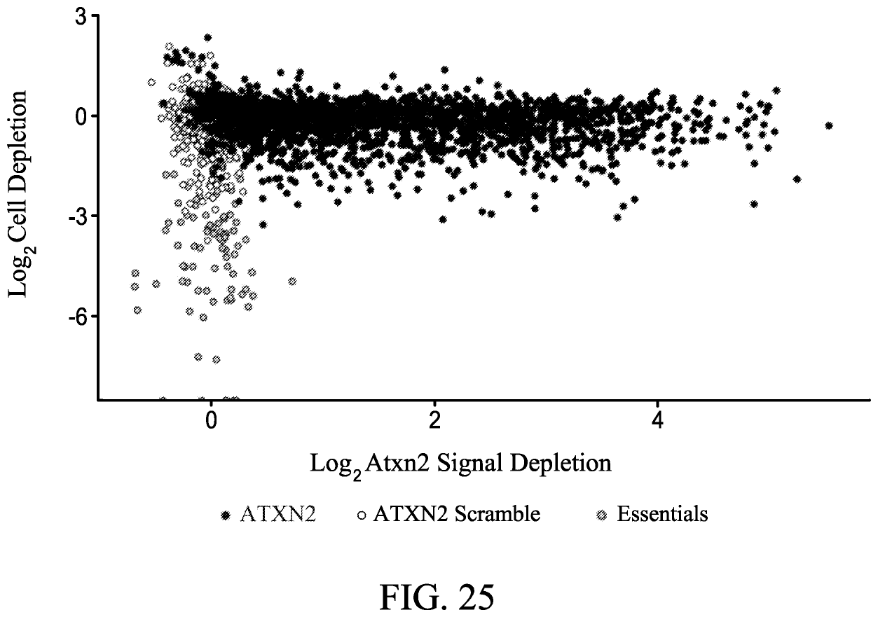

The ability of guide sequences to knock down ATXN2 and the presence of any altered proliferation or cytotoxicity were examined. FIG. 25 shows a plots of three classes of guide sequences in this experiment: ATXN2 targeting sequences, ATXN2 scrambled sequences, and amiRNAs targeting essential genes (predicted to be toxic). As expected, the log base-2 ATXN2 signal depletion (the scramble-baseline-corrected ATXN2 depletion in counts from high to low ATXN2 FACS conditions) was centered around 0 (no effect). However, many of these sequences exhibited remarkable shifts in abundance at a late collection timepoint, 16 days after transduction, versus an early timepoint after transduction. This is consistent with the reported essentiality for these sequences and demonstrates that this system can elicit cellular toxicity or proliferation impairment.

ATXN2 targeting guide sequences fall along a much wider spectrum along the axis of ATXN2 signal depletion compared to amiRNAs targeting essential genes or scrambled sequences, with targeting sequences exceeding 5 logs (base 2), corresponding to approximately 32-fold depletion of cells expressing these amiRNAs in high ATXN2 FACS collections versus low ATXN2 FACS collections.

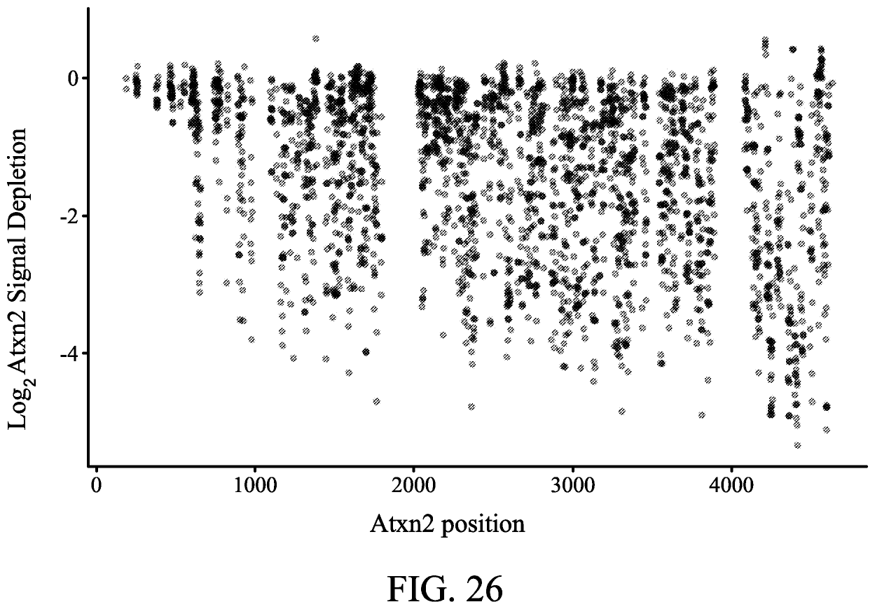

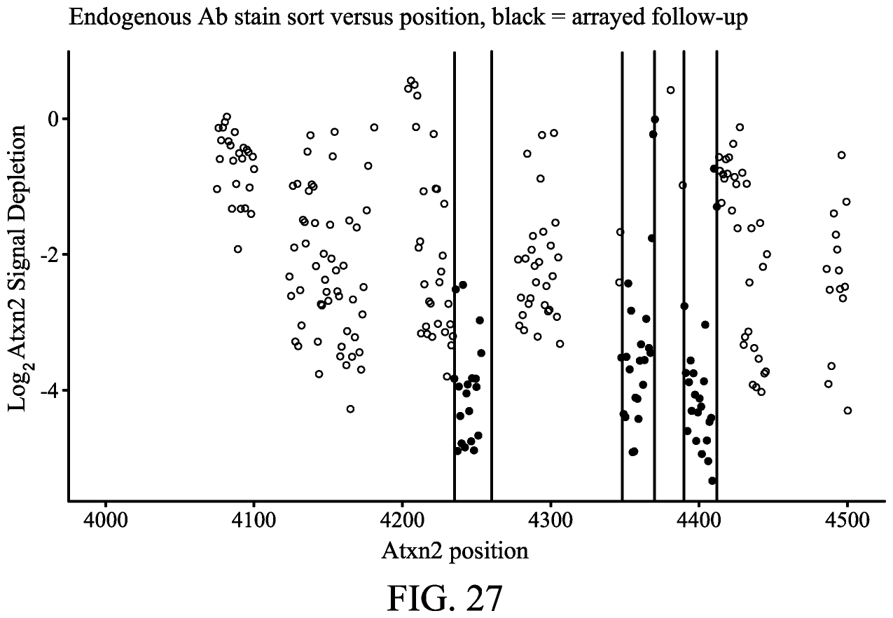

The near complete tiling of the ATXN2 transcript enables the detection of ‘hotspots’ of Ataxin-2 targeting guide sequences, defined by the proximity of their complementary regions of the Ataxin-2 transcript. FIG. 26 shows a plot of the knockdown efficacy, as measured by the depletion of counts for a given guide from the high ATXN2 FACS collection versus low ATXN2 FACS collection. Across the transcript, multiple regions where adjacent ATXN2 targeting guide sequences exhibit strong ATXN2 knockdown are noted. FIG. 27 shows a ‘zoom-in’ of regions within the 3′ UTR of ATXN2, and highlights guide sequences (as dark points) with unusually high ATXN2 lowering, as measured by the count reduction.

### Small RNAseq Confirmation of Pri-miRNA Processing Precision in the Pooled Screen

Guide sequences are excised from a miRNA stem by successive Drosha and Dicer processing. Each enzyme cuts the RNA. In the case of the miR backbone used for this tiled screen of ATXN2, the guide sequence from the corresponding endogenous miRNA (miR 16-2) is excised from the upstream, 5 prime arm, and therefore the guide sequence is cleaved from the parent stem at the 5′ side by Drosha. Because the position of the 5′ cut site determines the composition of the seed sequence, bases 2-7 counting from the 5′ nucleotide, the cutting position is important in determining both on- and off-target activity of the resulting guide sequence. Therefore, small RNAseq was conducted to assess the position of this cut.

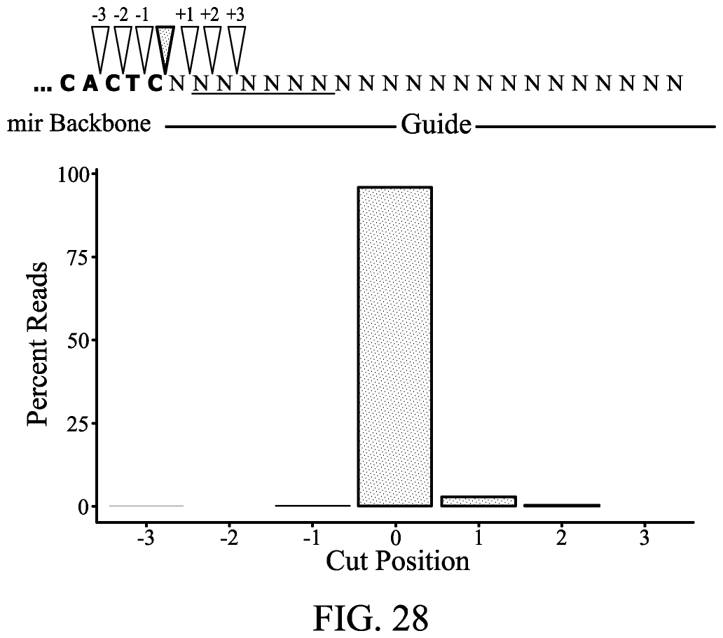

The tiling library, in packaged lentiviral form, was transduced at high multiplicity of infection into U2OS cells. After selection by puromycin to eliminate untransduced cells (the library vector contains a puromycin selection cassette), RNA was extracted by standard methods, and small RNA was purified and ligated with adapters to enable small RNA sequencing using the Nextflex small RNAseq kit v3. After PCR amplification, the resulting library was subject to next-generation sequencing on an Illumina MiSeq. A high proportion of reads had sequences of length 21, 22, and 23 nucleotides, with a peak at 22 nucleotides, consistent with the detection of processed miRNAs (guide and passenger sequences). To examine the precision of 5′ processing, the number of observations of 22-mer sequences matching several models of processed guide sequences were calculated. In one model, the guide sequence was assumed to be correctly processed. In other models, the guide sequence was assumed to be processed either upstream or downstream of the expected nucleotide. If the guide sequence is cut upstream of the intended nucleotide, then the expected upstream bases are incorporated from the miRNA backbone sequence. If the guide sequence is cut downstream of the intended nucleotide, then the first base of the resulting guide sequence is downstream of expected. Because the scrambled sequences in the library do not generally overlap from one another, for example, lowering the likelihood of ‘collisions’ where a guide sequence processed by excision from the stem at a nucleotide one downstream of the intended first nucleotide is the same as a guide sequence aligning to a position in the ATXN2 transcript one bp shifted, the processing position across all scrambling sequences was analyzed and averaged to estimate the most probable cutting position. FIG. 28 plots the percent of reads of the guide sequence with cut position at each nucleotide relative to the intended first nucleotide, and shows a very high proportion of reads begin at the intended position.

Additional ATXN2 Targeting Sequences from Pooled Screen

By examination of the knockdown efficacy against ATXN2 (as measured by depletion from the high versus low ATXN2 FACS collections) across the positions of complementarity to the ATXN2 transcript, several regions of interest were noted where clusters of high performing ATXN2-targeting guide sequences were observed. Table 19 lists these guide sequences, the targeting position of the guide sequences relative to the ATXN2 transcript (SEQ ID NO:2), the guide sequences inserted into the miRNA16-2 backbone (which are also the highest probability sequence that will be generated in the cell according to the above small RNAseq experiments), and the passenger sequences generated for the miR16-2 backbone. The guide sequences, miRNA16-2 formatted passenger sequences, and amiRNA sequences are provided in Table 19 in RNA format and DNA format (e.g., for insertion into a plasmid for AAV). Exemplary passenger RNA sequences (e.g., not modified for a specific miRNA backbone) are also provided in Table 19 in both RNA and DNA format. Efficacy of ATXN2 knockdown is represented by the signal depletion column. Altogether, sequences with high efficacy and low potential for dropout may represent good candidates to incorporate into therapeutic vectors targeting ATXN2.

TABLE 19
Guide sequences in ‘hot spots’ targeting ATXN2 from tiled screen and corresponding passenger and miRNA sequences

| | | | | | Mean | | | |
|---|---|---|---|---|---|---|---|---|
| | | | | | Atxn2 | | | |
| Guide | Guide | 16_2_format_ | 16_2_format_ | siRNA | Log2 | | | |
| Sequence | Sequence | passenger | passenger | Passenger | Signal | Atxn2 | amiRNA Sequence | amiRNA |
| (DNA) | (RNA) | (DNA) | (RNA) | (RNA) | Depletion | Position | (DNA) | Sequence (RNA) |
| TACCACAACAAA | UACCACAACAAA | ATGTTCAGACCC | AUGUUCAGACCC | AUGUUCAGACUU | -2.75633 | 1157 | ACCGGACATACTTGTT | ACCGGACAUACUUGUU |
| GTCTGAACAT | GUCUGAACAU | TGTTGTGGTT | UGUUGUGGUU | UGUUGUGG | | | CCACTCTACCACAACA | CCACUCUACCACAACA |
| [SEQ ID | [SEQ ID | [SEQ ID | [SEQ ID | [SEQ ID | | | AAGTCTGAACATTAGT | AAGUCUGAACAUUAGU |
| NO: 648] | NO: 1176] | NO: 761] | NO: 1289] | NO: 993] | | | GAAATATATATTAAAA | GAAAUAUAUAUUAAAA |
| | | | | | | | TGTTCAGACCCTGTTG | UGUUCAGACCCUGUUG |
| | | | | | | | TGGTTTAGTGTGACAG | UGGUUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 877] | [SEQ ID NO: 1405] |
| TTACCACAACAA | UUACCACAACAA | TGTTCAGACTCC | UGUUCAGACUCC | UGUUCAGACUUU | -2.02091 | 1158 | ACCGGACATACTTGTT | ACCGGACAUACUUGUU |
| AGTCTGAACA | AGUCUGAACA | GTTGTGGTAT | GUUGUGGUAU | GUUGUGGU | | | CCACTCTTACCACAAC | CCACUCUUACCACAAC |
| [SEQ ID | [SEQ ID | [SEQ ID | [SEQ ID | [SEQ ID | | | AAAGTCTGAACATAGT | AAAGUCUGAACAUAGU |
| NO: 649] | NO: 1177] | NO: 762] | NO: 1290] | NO: 994] | | | GAAATATATATTAAAT | GAAAUAUAUAUUAAAU |
| | | | | | | | GTTCAGACTCCGTTGT | GUUCAGACUCCGUUGU |
| | | | | | | | GGTATTAGTGTGACAG | GGUAUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 878] | [SEQ ID NO: 1406] |
| TGTACCACAACA | UGUACCACAACA | GTTCAGACTTCT | GUUCAGACUUCU | GUUCAGACUUUG | -3.6909 | 1159 | ACCGGACATACTTGTT | ACCGGACAUACUUGUU |
| AAGTCTGAAC | AAGUCUGAAC | TTGTGGTACT | UUGUGGUACU | UUGUGGUA | | | CCACTCTGTACCACAA | CCACUCUGUACCACAA |
| [SEQ ID | [SEQ ID | [SEQ ID | [SEQ ID | [SEQ ID | | | CAAAGTCTGAACTAGT | CAAAGUCUGAACUAGU |
| NO: 603] | NO: 40] | NO: 763] | NO: 1291] | NO: 995] | | | GAAATATATATTAAAG | GAAAUAUAUAUUAAAG |
| | (same as | | | | | | TTCAGACTTCTTTGTG | UUCAGACUUCUUUGUG |
| | guide in XD- | | | | | | GTACTTAGTGTGACAG | GUACUUAGUGUGACAG |
| | 14756) | | | | | | GGATACAGCAACTATT | GGAUACAGCAACUAUU |
| | | | | | | | TTATCAA | UUAUCAA |
| | | | | | | | [SEQ ID NO: 879] | [SEQ ID NO: 1407] |
| TTGTACCACAAC | UUGUACCACAAC | TTCAGACTTTTC | UUCAGACUUUUC | UUCAGACUUUGU | -1.52769 | 1160 | ACCGGACATACTTGTT | ACCGGACAUACUUGUU |
| AAAGTCTGAA | AAAGUCUGAA | TGTGGTACAT | UGUGGUACAU | UGUGGUAC | | | CCACTCTTGTACCACA | CCACUCUUGUACCACA |
| [SEQ ID | [SEQ ID | [SEQ ID | [SEQ ID | [SEQ ID | | | ACAAAGTCTGAATAGT | ACAAAGUCUGAAUAGU |
| NO: 650] | NO: 1178] | NO: 764] | NO: 1292] | NO: 996] | | | GAAATATATATTAAAT | GAAAUAUAUAUUAAAU |
| | | | | | | | TCAGACTTTTCTGTGG | UCAGACUUUUCUGUGG |
| | | | | | | | TACATTAGTGTGACAG | UACAUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 880] | [SEQ ID NO: 1408] |
| ACTGTACCACAA | ACUGUACCACAA | TCAGACTTTGCC | UCAGACUUUGCC | UCAGACUUUGUU | -3.46564 | 1161 | ACCGGACATACTTGTT | ACCGGACAUACUUGUU |
| CAAAGTCTGA | CAAAGUCUGA | GTGGTACAGA | GUGGUACAGA | GUGGUACA | | | CCACTCACTGTACCAC | CCACUCACUGUACCAC |
| [SEQ ID | [SEQ ID | [SEQ ID | [SEQ ID | [SEQ ID | | | AACAAAGTCTGATAGT | AACAAAGUCUGAUAGU |
| NO: 651] | NO: 1179] | NO: 765] | NO: 1293] | NO: 997] | | | GAAATATATATTAAAT | GAAAUAUAUAUUAAAU |
| | | | | | | | CAGACTTTGCCGTGGT | CAGACUUUGCCGUGGU |
| | | | | | | | ACAGATAGTGTGACAG | ACAGAUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 881] | [SEQ ID NO: 1409] |
| AACTGTACCACA | AACUGUACCACA | CAGACTTTGTCT | CAGACUUUGUCU | CAGACUUUGUUG | -3.89476 | 1162 | ACCGGACATACTTGTT | ACCGGACAUACUUGUU |
| ACAAAGTCTG | ACAAAGUCUG | TGGTACAGTA | UGGUACAGUA | UGGUACAG | | | CCACTCAACTGTACCA | CCACUCAACUGUACCA |
| [SEQ ID | [SEQ ID | [SEQ ID | [SEQ ID | [SEQ ID | | | CAACAAAGTCTGTAGT | CAACAAAGUCUGUAGU |
| NO: 652] | NO: 1180] | NO: 766] | NO: 1294] | NO: 998] | | | GAAATATATATTAAAC | GAAAUAUAUAUUAAAC |
| | | | | | | | AGACTTTGTCTTGGTA | AGACUUUGUCUUGGUA |
| | | | | | | | CAGTATAGTGTGACAG | CAGUAUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 882] | [SEQ ID NO: 1410] |
| AAACTGTACCAC | AAACUGUACCAC | AGACTTTGTTTC | AGACUUUGUUUC | AGACUUUGUUGU | -2.62277 | 1163 | ACCGGACATACTTGTT | ACCGGACAUACUUGUU |
| AACAAAGTCT | AACAAAGUCU | GGTACAGTTA | GGUACAGUUA | GGUACAGU | | | CCACTCAAACTGTACC | CCACUCAAACUGUACC |
| [SEQ ID | [SEQ ID | [SEQ ID | [SEQ ID | [SEQ ID | | | ACAACAAAGTCTTAGT | ACAACAAAGUCUUAGU |
| NO: 653] | NO: 1181] | NO: 767] | NO: 1295 | NO: 999] | | | GAAATATATATTAAAA | GAAAUAUAUAUUAAAA |
| | | | | | | | GACTTTGTTTCGGTAC | GACUUUGUUUCGGUAC |
| | | | | | | | AGTTATAGTGTGACAG | AGUUAUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 883] | [SEQ ID NO: 1411] |
| TTAAACTGTACC | UUAAACUGUACC | ACTTTGTTGTTT | ACUUUGUUGUUU | ACUUUGUUGUGG | -0.49686 | 1165 | ACCGGACATACTTGTT | ACCGGACAUACUUGUU |
| ACAACAAAGT | ACAACAAAGU | TACAGTTTAT | UACAGUUUAU | UACAGUUU | | | CCACTCTTAAACTGTA | CCACUCUUAAACUGUA |
| [SEQ ID | [SEQ ID | [SEQ ID | [SEQ ID | [SEQ ID | | | CCACAACAAAGTTAGT | CCACAACAAAGUUAGU |
| NO: 654] | NO: 1182] | NO: 768] | NO: 1296] | NO: 1000] | | | GAAATATATATTAAAA | GAAAUAUAUAUUAAAA |
| | (same as | | | | | | CTTTGTTGTTTTACAG | CUUUGUUGUUUUACAG |
| | guide in XD- | | | | | | TTTATTAGTGTGACAG | UUUAUUAGUGUGACAG |
| | 14757) | | | | | | GGATACAGCAACTATT | GGAUACAGCAACUAUU |
| | | | | | | | TTATCAA | UUAUCAA |
| | | | | | | | [SEQ ID NO: 884] | [SEQ ID NO: 1412] |
| TTGCTAACTGGT | UUGCUAACUGGU | GCAAGGGCAAGA | GCAAGGGCAAGA | GCAAGGGCAAAC | -3.48781 | 1479 | ACCGGACATACTTGTT | ACCGGACAUACUUGUU |
| TTGCCCTTGC | UUGCCCUUGC | CAGTTAGCAT | CAGUUAGCAU | CAGUUAGC | | | CCACTCTTGCTAACTG | CCACUCUUGCUAACUG |
| [SEQ ID | [SEQ ID | [SEQ ID | [SEQ ID | [SEQ ID | | | GTTTGCCCTTGCTAGT | GUUUGCCCUUGCUAGU |
| NO: 655] | NO: 1183] | NO: 769] | NO: 1297] | NO: 1001] | | | GAAATATATATTAAAG | GAAAUAUAUAUUAAAG |
| | | | | | | | CAAGGGCAAGACAGTT | CAAGGGCAAGACAGUU |
| | | | | | | | AGCATTAGTGTGACAG | AGCAUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 885] | [SEQ ID NO: 1413] |
| TGGGTTGAAATC | UGGGUUGAAAUC | TCACACTTCATG | UCACACUUCAUG | UCACACUUCAGA | -1.9344 | 1754 | ACCGGACATACTTGTT | ACCGGACAUACUUGUU |
| TGAAGTGTGA | UGAAGUGUGA | TTTCAACCCT | UUUCAACCCU | UUUCAACC | | | CCACTCTGGGTTGAAA | CCACUCUGGGUUGAAA |
| [SEQ ID | [SEQ ID | [SEQ ID | [SEQ ID | [SEQ ID | | | TCTGAAGTGTGATAGT | UCUGAAGUGUGAUAGU |
| NO: 656] | NO: 1184] | NO: 770] | NO: 1298] | NO: 1002] | | | GAAATATATATTAAAT | GAAAUAUAUAUUAAAU |
| | | | | | | | CACACTTCATGTTTCA | CACACUUCAUGUUUCA |
| | | | | | | | ACCCTTAGTGTGACAG | ACCCUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 886] | [SEQ ID NO: 1414] |
| TCGGGTTGAAAT | UCGGGUUGAAAU | CACACTTCAGGC | CACACUUCAGGC | CACACUUCAGAU | -4.71279 | 1755 | ACCGGACATACTTGTT | ACCGGACAUACUUGUU |
| CTGAAGTGTG | CUGAAGUGUG | TTCAACCCGT | UUCAACCCGU | UUCAACCC | | | CCACTCTCGGGTTGAA | CCACUCUCGGGUUGAA |
| [SEQ ID | [SEQ ID | [SEQ ID | [SEQ ID | [SEQ ID | | | ATCTGAAGTGTGTAGT | AUCUGAAGUGUGUAGU |
| NO: 657] | NO: 1185] | NO: 771] | NO: 1299] | NO: 1003] | | | GAAATATATATTAAAC | GAAAUAUAUAUUAAAC |
| | | | | | | | ACACTTCAGGCTTCAA | ACACUUCAGGCUUCAA |
| | | | | | | | CCCGTTAGTGTGACAG | CCCGUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 887] | [SEQ ID NO: 1415] |
| TTCGGGTTGAAA | UUCGGGUUGAAA | ACACTTCAGACC | ACACUUCAGACC | ACACUUCAGAUU | -3.7055 | 1756 | ACCGGACATACTTGTT | ACCGGACAUACUUGUU |
| TCTGAAGTGT | UCUGAAGUGU | TCAACCCGAT | UCAACCCGAU | UCAACCCG | | | CCACTCTTCGGGTTGA | CCACUCUUCGGGUUGA |
| [SEQ ID | [SEQ ID | [SEQ ID | [SEQ ID | [SEQ ID | | | AATCTGAAGTGTTAGT | AAUCUGAAGUGUUAGU |
| NO: 607] | NO: 108] | NO: 772] | NO: 1300] | NO: 1004] | | | GAAATATATATTAAAA | GAAAUAUAUAUUAAAA |
| | (same as | | | | | | CACTTCAGACCTCAAC | CACUUCAGACCUCAAC |
| | guide in XD- | | | | | | CCGATTAGTGTGACAG | CCGAUUAGUGUGACAG |
| | 14790) | | | | | | GGATACAGCAACTATT | GGAUACAGCAACUAUU |
| | | | | | | | TTATCAA | UUAUCAA |
| | | | | | | | [SEQ ID NO: 888] | [SEQ ID NO: 1416] |
| TGATGCAGGACT | UGAUGCAGGACU | TACGCCTGCTGT | UACGCCUGCUGU | UACGCCUGCUAG | -1.91676 | 2351 | ACCGGACATACTTGTT | ACCGGACAUACUUGUU |
| AGCAGGCGTA | AGCAGGCGUA | TCCTGCATCT | UCCUGCAUCU | UCCUGCAU | | | CCACTCTGATGCAGGA | CCACUCUGAUGCAGGA |
| [SEQ ID | [SEQ ID | [SEQ ID | [SEQ ID | [SEQ ID | | | CTAGCAGGCGTATAGT | CUAGCAGGCGUAUAGU |
| NO: 658] | NO: 1186] | NO: 773] | NO: 1301] | NO: 1005] | | | GAAATATATATTAAAT | GAAAUAUAUAUUAAAU |
| | | | | | | | ACGCCTGCTGTTCCTG | ACGCCUGCUGUUCCUG |
| | | | | | | | CATCTTAGTGTGACAG | CAUCUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 889] | [SEQ ID NO: 1417] |
| TCGATGCAGGAC | UCGAUGCAGGAC | ACGCCTGCTATC | ACGCCUGCUAUC | ACGCCUGCUAGU | -2.27165 | 2352 | ACCGGACATACTTGTT | ACCGGACAUACUUGUU |
| TAGCAGGCGT | UAGCAGGCGU | CCTGCATCGT | CCUGCAUCGU | CCUGCAUC | | | CCACTCTCGATGCAGG | CCACUCUCGAUGCAGG |
| [SEQ ID | [SEQ ID | [SEQ ID | [SEQ ID | [SEQ ID | | | ACTAGCAGGCGTTAGT | ACUAGCAGGCGUUAGU |
| NO: 659] | NO: 1187] | NO: 774] | NO: 1302] | NO: 1006] | | | GAAATATATATTAAAA | GAAAUAUAUAUUAAAA |
| | | | | | | | CGCCTGCTATCCCTGC | CGCCUGCUAUCCCUGC |
| | | | | | | | ATCGTTAGTGTGACAG | AUCGUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 890] | [SEQ ID NO: 1418] |
| TTCGATGCAGGA | UUCGAUGCAGGA | CGCCTGCTAGCA | CGCCUGCUAGCA | CGCCUGCUAGUC | -4.78943 | 2353 | ACCGGACATACTTGTT | ACCGGACAUACUUGUU |
| CTAGCAGGCG | CUAGCAGGCG | CTGCATCGAT | CUGCAUCGAU | CUGCAUCG | | | CCACTCTTCGATGCAG | CCACUCUUCGAUGCAG |
| [SEQ ID | [SEQ ID | [SEQ ID | [SEQ ID | [SEQ ID | | | GACTAGCAGGCGTAGT | GACUAGCAGGCGUAGU |
| NO: 612] | NO: 166] | NO: 775] | NO: 1303] | NO: 1007] | | | GAAATATATATTAAAC | GAAAUAUAUAUUAAAC |
| | (same as | | | | | | GCCTGCTAGCACTGCA | GCCUGCUAGCACUGCA |
| | guide in XD- | | | | | | TCGATTAGTGTGACAG | UCGAUUAGUGUGACAG |
| | 14819) | | | | | | GGATACAGCAACTATT | GGAUACAGCAACUAUU |
| | | | | | | | TTATCAA | UUAUCAA |
| | | | | | | | [SEQ ID NO: 891] | [SEQ ID NO: 1419] |
| TTTCGATGCAGG | UUUCGAUGCAGG | GCCTGCTAGTAA | GCCUGCUAGUAA | GCCUGCUAGUCC | -3.98959 | 2354 | ACCGGACATACTTGTT | ACCGGACAUACUUGUU |
| ACTAGCAGGC | ACUAGCAGGC | TGCATCGAAT | UGCAUCGAAU | UGCAUCGA | | | CCACTCTTTCGATGCA | CCACUCUUUCGAUGCA |
| [SEQ ID | [SEQ ID | [SEQ ID | [SEQ ID | [SEQ ID | | | GGACTAGCAGGCTAGT | GGACUAGCAGGCUAGU |
| NO: 660] | NO: 1188] | NO: 776] | NO: 1304] | NO: 1008] | | | GAAATATATATTAAAG | GAAAUAUAUAUUAAAG |
| | | | | | | | CCTGCTAGTAATGCAT | CCUGCUAGUAAUGCAU |
| | | | | | | | CGAATTAGTGTGACAG | CGAAUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 892] | [SEQ ID NO: 1420] |
| TGTTCGATGCAG | UGUUCGAUGCAG | CCTGCTAGTCAC | CCUGCUAGUCAC | CCUGCUAGUCCU | -3.78415 | 2355 | ACCGGACATACTTGTT | ACCGGACAUACUUGUU |
| GACTAGCAGG | GACUAGCAGG | GCATCGAACT | GCAUCGAACU | GCAUCGAA | | | CCACTCTGTTCGATGC | CCACUCUGUUCGAUGC |
| [SEQ ID | [SEQ ID | [SEQ ID | [SEQ ID | [SEQ ID | | | AGGACTAGCAGGTAGT | AGGACUAGCAGGUAGU |
| NO: 661] | NO: 1189] | NO: 777] | NO: 1305] | NO: 1009] | | | GAAATATATATTAAAC | GAAAUAUAUAUUAAAC |
| | (same as | | | | | | CTGCTAGTCACGCATC | CUGCUAGUCACGCAUC |
| | guide in XD- | | | | | | GAACTTAGTGTGACAG | GAACUUAGUGUGACAG |
| | 14820) | | | | | | GGATACAGCAACTATT | GGAUACAGCAACUAUU |
| | | | | | | | TTATCAA | UUAUCAA |
| | | | | | | | [SEQ ID NO: 893] | [SEQ ID NO: 1421] |
| TTGTTCGATGCA | UUGUUCGAUGCA | CTGCTAGTCCCT | CUGCUAGUCCCU | CUGCUAGUCCUG | -2.10173 | 2356 | ACCGGACATACTTGTT | ACCGGACAUACUUGUU |
| GGACTAGCAG | GGACUAGCAG | CATCGAACAT | CAUCGAACAU | CAUCGAAC | | | CCACTCTTGTTCGATG | CCACUCUUGUUCGAUG |
| [SEQ ID | [SEQ ID | [SEQ ID | [SEQ ID | [SEQ ID | | | CAGGACTAGCAGTAGT | CAGGACUAGCAGUAGU |
| NO: 662] | NO: 1190] | NO: 778] | NO: 1306] | NO: 1010] | | | GAAATATATATTAAAC | GAAAUAUAUAUUAAAC |
| | | | | | | | TGCTAGTCCCTCATCG | UGCUAGUCCCUCAUCG |
| | | | | | | | AACATTAGTGTGACAG | AACAUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 894] | [SEQ ID NO: 1422] |
| TCTGTTCGATGC | UCUGUUCGAUGC | TGCTAGTCCTTA | UGCUAGUCCUUA | UGCUAGUCCUGC | -1.98657 | 2357 | ACCGGACATACTTGTT | ACCGGACAUACUUGUU |
| AGGACTAGCA | AGGACUAGCA | ATCGAACAGT | AUCGAACAGU | AUCGAACA | | | CCACTCTCTGTTCGAT | CCACUCUCUGUUCGAU |
| [SEQ ID | [SEQ ID | [SEQ ID | [SEQ ID | [SEQ ID | | | GCAGGACTAGCATAGT | GCAGGACUAGCAUAGU |
| NO: 663] | NO: 1191] | NO: 779] | NO: 1307] | NO: 1011] | | | GAAATATATATTAAAT | GAAAUAUAUAUUAAAU |
| | | | | | | | GCTAGTCCTTAATCGA | GCUAGUCCUUAAUCGA |
| | | | | | | | ACAGTTAGTGTGACAG | ACAGUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 895] | [SEQ ID NO: 1423] |
| TTCTGTTCGATG | UUCUGUUCGAUG | GCTAGTCCTGAG | GCUAGUCCUGAG | GCUAGUCCUGCA | -4.1672 | 2358 | ACCGGACATACTTGTT | ACCGGACAUACUUGUU |
| CAGGACTAGC | CAGGACUAGC | TCGAACAGAT | UCGAACAGAU | UCGAACAG | | | CCACTCTTCTGTTCGA | CCACUCUUCUGUUCGA |
| [SEQ ID | [SEQ ID | [SEQ ID | [SEQ ID | [SEQ ID | | | TGCAGGACTAGCTAGT | UGCAGGACUAGCUAGU |
| NO: 664] | NO: 1192] | NO: 780] | NO: 1308] | NO: 1012] | | | GAAATATATATTAAAG | GAAAUAUAUAUUAAAG |
| | | | | | | | CTAGTCCTGAGTCGAA | CUAGUCCUGAGUCGAA |
| | | | | | | | CAGATTAGTGTGACAG | CAGAUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 896] | [SEQ ID NO: 1424] |
| TCTCTGTTCGAT | UCUCUGUUCGAU | CTAGTCCTGCGC | CUAGUCCUGCGC | CUAGUCCUGCAU | -1.7173 | 2359 | ACCGGACATACTTGTT | ACCGGACAUACUUGUU |
| GCAGGACTAG | GCAGGACUAG | CGAACAGAGT | CGAACAGAGU | CGAACAGA | | | CCACTCTCTCTGTTCG | CCACUCUCUCUGUUCG |
| [SEQ ID | [SEQ ID | [SEQ ID | [SEQ ID | [SEQ ID | | | ATGCAGGACTAGTAGT | AUGCAGGACUAGUAGU |
| NO: 665] | NO: 1193] | NO: 781] | NO: 1309] | NO: 1013] | | | GAAATATATATTAAAC | GAAAUAUAUAUUAAAC |
| | | | | | | | TAGTCCTGCGCCGAAC | UAGUCCUGCGCCGAAC |
| | | | | | | | AGAGTTAGTGTGACAG | AGAGUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 897] | [SEQ ID NO: 1425] |
| TGAGAGAAGGAA | UGAGAGAAGGAA | TCAACCCACGCC | UCAACCCACGCC | UCAACCCACGUU | -0.42433 | 2926 | ACCGGACATACTTGTT | ACCGGACAUACUUGUU |
| CGTGGGTTGA | CGUGGGUUGA | CCTTCTCTCT | CCUUCUCUCU | CCUUCUCU | | | CCACTCTGAGAGAAGG | CCACUCUGAGAGAAGG |
| [SEQ ID | [SEQ ID | [SEQ ID | [ SEQ ID | [SEQ ID | | | AACGTGGGTTGATAGT | AACGUGGGUUGAUAGU |
| NO: 666] | NO: 1194] | NO: 782] | NO: 1310] | NO: 1014] | | | GAAATATATATTAAAT | GAAAUAUAUAUUAAAU |
| | | | | | | | CAACCCACGCCCCTTC | CAACCCACGCCCCUUC |
| | | | | | | | TCTCTTAGTGTGACAG | UCUCUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 898] | [SEQ ID NO: 1426] |
| TTGAGAGAAGGA | UUGAGAGAAGGA | CAACCCACGTCA | CAACCCACGUCA | CAACCCACGUUC | -3.38604 | 2927 | ACCGGACATACTTGTT | ACCGGACAUACUUGUU |
| ACGTGGGTTG | ACGUGGGUUG | CTTCTCTCAT | CUUCUCUCAU | CUUCUCUC | | | CCACTCTTGAGAGAAG | CCACUCUUGAGAGAAG |
| [SEQ ID | [SEQ ID | [SEQ ID | [SEQ ID | [SEQ ID | | | GAACGTGGGTTGTAGT | GAACGUGGGUUGUAGU |
| NO: 667] | NO: 1195] | NO: 783] | NO: 1311] | NO: 1015] | | | GAAATATATATTAAAC | GAAAUAUAUAUUAAAC |
| | | | | | | | AACCCACGTCACTTCT | AACCCACGUCACUUCU |
| | | | | | | | CTCATTAGTGTGACAG | CUCAUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 899] | [SEQ ID NO: 1427] |
| TCTGAGAGAAGG | UCUGAGAGAAGG | AACCCACGTTAA | AACCCACGUUAA | AACCCACGUUCC | -3.5546 | 2928 | ACCGGACATACTTGTT | ACCGGACAUACUUGUU |
| AACGTGGGTT | AACGUGGGUU | TTCTCTCAGT | UUCUCUCAGU | UUCUCUCA | | | CCACTCTCTGAGAGAA | CCACUCUCUGAGAGAA |
| [SEQ ID | [SEQ ID | [SEQ ID | [SEQ ID | [SEQ ID | | | GGAACGTGGGTTTAGT | GGAACGUGGGUUUAGU |
| NO: 668] | NO: 1196] | NO: 784] | NO: 1312] | NO: 1016] | | | GAAATATATATTAAAA | GAAAUAUAUAUUAAAA |
| | | | | | | | ACCCACGTTAATTCTC | ACCCACGUUAAUUCUC |
| | | | | | | | TCAGTTAGTGTGACAG | UCAGUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 900] | [SEQ ID NO: 1428] |
| TGCTGAGAGAAG | UGCUGAGAGAAG | ACCCACGTTCAC | ACCCACGUUCAC | ACCCACGUUCCU | -2.96935 | 2929 | ACCGGACATACTTGTT | ACCGGACAUACUUGUU |
| GAACGTGGGT | GAACGUGGGU | TCTCTCAGCT | UCUCUCAGCU | UCUCUCAG | | | CCACTCTGCTGAGAGA | CCACUCUGCUGAGAGA |
| [SEQ ID | [SEQ ID | [SEQ ID | [SEQ ID | [SEQ ID | | | AGGAACGTGGGTTAGT | AGGAACGUGGGUUAGU |
| NO: 669] | NO: 1197] | NO: 785] | NO: 1313] | NO: 1017] | | | GAAATATATATTAAAA | GAAAUAUAUAUUAAAA |
| | | | | | | | CCCACGTTCACTCTCT | CCCACGUUCACUCUCU |
| | | | | | | | CAGCTTAGTGTGACAG | CAGCUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 901] | [SEQ ID NO: 1429] |
| TGGCTGAGAGAA | UGGCUGAGAGAA | CCCACGTTCCCC | CCCACGUUCCCC | CCCACGUUCCUU | -1.84629 | 2930 | ACCGGACATACTTGTT | ACCGGACAUACUUGUU |
| GGAACGTGGG | GGAACGUGGG | CTCTCAGCCT | CUCUCAGCCU | CUCUCAGC | | | CCACTCTGGCTGAGAG | CCACUCUGGCUGAGAG |
| [SEQ ID | [SEQ ID | [SEQ ID | [SEQ ID | [SEQ ID | | | AAGGAACGTGGGTAGT | AAGGAACGUGGGUAGU |
| NO: 670] | NO: 1198] | NO: 786] | NO: 1314] | NO: 1018] | | | GAAATATATATTAAAC | GAAAUAUAUAUUAAAC |
| | | | | | | | CCACGTTCCCCCTCTC | CCACGUUCCCCCUCUC |
| | | | | | | | AGCCTTAGTGTGACAG | AGCCUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 902] | [SEQ ID NO: 1430] |
| TTGGCTGAGAGA | UUGGCUGAGAGA | CCACGTTCCTCA | CCACGUUCCUCA | CCACGUUCCUUC | -4.19621 | 2931 | ACCGGACATACTTGTT | ACCGGACAUACUUGUU |
| AGGAACGTGG | AGGAACGUGG | TCTCAGCCAT | UCUCAGCCAU | UCUCAGCC | | | CCACTCTTGGCTGAGA | CCACUCUUGGCUGAGA |
| [SEQ ID | [SEQ ID | [SEQ ID | [SEQ ID | [SEQ ID | | | GAAGGAACGTGGTAGT | GAAGGAACGUGGUAGU |
| NO: 671] | NO: 1199] | NO: 787] | NO: 1315] | NO: 1019] | | | GAAATATATATTAAAC | GAAAUAUAUAUUAAAC |
| | | | | | | | CACGTTCCTCATCTCA | CACGUUCCUCAUCUCA |
| | | | | | | | GCCATTAGTGTGACAG | GCCAUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 903] | [SEQ ID NO: 1431] |
| TTTGGCTGAGAG | UUUGGCUGAGAG | CACGTTCCTTAC | CACGUUCCUUAC | CACGUUCCUUCU | -3.26413 | 2932 | ACCGGACATACTTGTT | ACCGGACAUACUUGUU |
| AAGGAACGTG | AAGGAACGUG | CTCAGCCAAT | CUCAGCCAAU | CUCAGCCA | | | CCACTCTTTGGCTGAG | CCACUCUUUGGCUGAG |
| [SEQ ID | [SEQ ID | [SEQ ID | [SEQ ID | [SEQ ID | | | AGAAGGAACGTGTAGT | AGAAGGAACGUGUAGU |
| NO: 672] | NO: 1200] | NO: 788] | NO: 1316] | NO: 1020] | | | GAAATATATATTAAAC | GAAAUAUAUAUUAAAC |
| | | | | | | | ACGTTCCTTACCTCAG | ACGUUCCUUACCUCAG |
| | | | | | | | CCAATTAGTGTGACAG | CCAAUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 904] | [SEQ ID NO: 1432] |
| TTTTGGCTGAGA | UUUUGGCUGAGA | ACGTTCCTTCCA | ACGUUCCUUCCA | ACGUUCCUUCUC | -0.25972 | 2933 | ACCGGACATACTTGTT | ACCGGACAUACUUGUU |
| GAAGGAACGT | GAAGGAACGU | TCAGCCAAAT | UCAGCCAAAU | UCAGCCAA | | | CCACTCTTTTGGCTGA | CCACUCUUUUGGCUGA |
| [SEQ ID | [SEQ ID | [SEQ ID | [SEQ ID | [SEQ ID | | | GAGAAGGAACGTTAGT | GAGAAGGAACGUUAGU |
| NO: 673] | NO: 1201] | NO: 789] | NO: 1317] | NO: 1021] | | | GAAATATATATTAAAA | GAAAUAUAUAUUAAAA |
| | | | | | | | CGTTCCTTCCATCAGC | CGUUCCUUCCAUCAGC |
| | | | | | | | CAAATTAGTGTGACAG | CAAAUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 905] | [SEQ ID NO: 1433] |
| TCTTTGGCTGAG | UCUUUGGCUGAG | CGTTCCTTCTAC | CGUUCCUUCUAC | CGUUCCUUCUCU | -0.75797 | 2934 | ACCGGACATACTTGTT | ACCGGACAUACUUGUU |
| AGAAGGAACG | AGAAGGAACG | CAGCCAAAGT | CAGCCAAAGU | CAGCCAAA | | | CCACTCTCTTTGGCTG | CCACUCUCUUUGGCUG |
| [SEQ ID | [SEQ ID | [SEQ ID | [SEQ ID | [SEQ ID | | | AGAGAAGGAACGTAGT | AGAGAAGGAACGUAGU |
| NO: 674] | NO: 1202] | NO: 790] | NO: 1318] | NO: 1022] | | | GAAATATATATTAAAC | GAAAUAUAUAUUAAAC |
| | | | | | | | GTTCCTTCTACCAGCC | GUUCCUUCUACCAGCC |
| | | | | | | | AAAGTTAGTGTGACAG | AAAGUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 906] | [SEQ ID NO: 1434] |
| TGCTTTGGCTGA | UGCUUUGGCUGA | GTTCCTTCTCCA | GUUCCUUCUCCA | GUUCCUUCUCUC | -1.45481 | 2935 | ACCGGACATACTTGTT | ACCGGACAUACUUGUU |
| GAGAAGGAAC | GAGAAGGAAC | AGCCAAAGCT | AGCCAAAGCU | AGCCAAAG | | | CCACTCTGCTTTGGCT | CCACUCUGCUUUGGCU |
| [SEQ ID | [SEQ ID | [SEQ ID | [SEQ ID | [SEQ ID | | | GAGAGAAGGAACTAGT | GAGAGAAGGAACUAGU |
| NO: 675] | NO: 1203] | NO: 791] | NO: 1319] | NO: 1023] | | | GAAATATATATTAAAG | GAAAUAUAUAUUAAAG |
| | | | | | | | TTCCTTCTCCAAGCCA | UUCCUUCUCCAAGCCA |
| | | | | | | | AAGCTTAGTGTGACAG | AAGCUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 907] | [SEQ ID NO: 1435] |
| AGGCTTTGGCTG | AGGCUUUGGCUG | TTCCTTCTCTAG | UUCCUUCUCUAG | UUCCUUCUCUCA | -0.85197 | 2936 | ACCGGACATACTTGTT | ACCGGACAUACUUGUU |
| AGAGAAGGAA | AGAGAAGGAA | GCCAAAGCCA | GCCAAAGCCA | GCCAAAGC | | | CCACTCAGGCTTTGGC | CCACUCAGGCUUUGGC |
| [SEQ ID | [SEQ ID | [SEQ ID | [SEQ ID | [SEQ ID | | | TGAGAGAAGGAATAGT | UGAGAGAAGGAAUAGU |
| NO: 676] | NO: 1204] | NO: 792] | NO: 1320] | NO: 1024] | | | GAAATATATATTAAAT | GAAAUAUAUAUUAAAU |
| | | | | | | | TCCTTCTCTAGGCCAA | UCCUUCUCUAGGCCAA |
| | | | | | | | AGCCATAGTGTGACAG | AGCCAUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 908] | [SEQ ID NO: 1436] |
| AAGGCTTTGGCT | AAGGCUUUGGCU | TCCTTCTCTCGT | UCCUUCUCUCGU | UCCUUCUCUCAG | -3.87114 | 2937 | ACCGGACATACTTGTT | ACCGGACAUACUUGUU |
| GAGAGAAGGA | GAGAGAAGGA | CCAAAGCCTA | CCAAAGCCUA | CCAAAGCC | | | CCACTCAAGGCTTTGG | CCACUCAAGGCUUUGG |
| [SEQ ID | [SEQ ID | [SEQ ID | [SEQ ID | [SEQ ID | | | CTGAGAGAAGGATAGT | CUGAGAGAAGGAUAGU |
| NO: 677] | NO: 1205] | NO: 793] | NO: 1321] | NO: 1025] | | | GAAATATATATTAAAT | GAAAUAUAUAUUAAAU |
| | | | | | | | CCTTCTCTCGTCCAAA | CCUUCUCUCGUCCAAA |
| | | | | | | | GCCTATAGTGTGACAG | GCCUAUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 909] | [SEQ ID NO: 1437] |
| TAAGGCTTTGGC | UAAGGCUUUGGC | CCTTCTCTCATA | CCUUCUCUCAUA | CCUUCUCUCAGC | -0.34984 | 2938 | ACCGGACATACTTGTT | ACCGGACAUACUUGUU |
| TGAGAGAAGG | UGAGAGAAGG | CAAAGCCTTT | CAAAGCCUUU | CAAAGCCU | | | CCACTCTAAGGCTTTG | CCACUCUAAGGCUUUG |
| [SEQ ID | [SEQ ID | [SEQ ID | [SEQ ID | [SEQ ID | | | GCTGAGAGAAGGTAGT | GCUGAGAGAAGGUAGU |
| NO: 678] | NO: 1206] | NO: 794] | NO: 1322] | NO: 1026] | | | GAAATATATATTAAAC | GAAAUAUAUAUUAAAC |
| | | | | | | | CTTCTCTCATACAAAG | CUUCUCUCAUACAAAG |
| | | | | | | | CCTTTTAGTGTGACAG | CCUUUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 910] | [SEQ ID NO: 1438] |
| AGAAGGCTTTGG | AGAAGGCUUUGG | CTTCTCTCAGAA | CUUCUCUCAGAA | CUUCUCUCAGCC | -2.37082 | 2939 | ACCGGACATACTTGTT | ACCGGACAUACUUGUU |
| CTGAGAGAAG | CUGAGAGAAG | AAAGCCTTCA | AAAGCCUUCA | AAAGCCUU | | | CCACTCAGAAGGCTTT | CCACUCAGAAGGCUUU |
| [SEQ ID | [SEQ ID | [SEQ ID | [SEQ ID | [SEQ ID | | | GGCTGAGAGAAGTAGT | GGCUGAGAGAAGUAGU |
| NO: 679] | NO: 1207] | NO: 795] | NO: 1323] | NO: 1027] | | | GAAATATATATTAAAC | GAAAUAUAUAUUAAAC |
| | | | | | | | TTCTCTCAGAAAAAGC | UUCUCUCAGAAAAAGC |
| | | | | | | | CTTCATAGTGTGACAG | CUUCAUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 911] | [SEQ ID NO: 1439] |
| TAGAAGGCTTTG | UAGAAGGCUUUG | TTCTCTCAGCAG | UUCUCUCAGCAG | UUCUCUCAGCCA | -0.15876 | 2940 | ACCGGACATACTTGTT | ACCGGACAUACUUGUU |
| GCTGAGAGAA | GCUGAGAGAA | AAGCCTTCTT | AAGCCUUCUU | AAGCCUUC | | | CCACTCTAGAAGGCTT | CCACUCUAGAAGGCUU |
| [SEQ ID | [SEQ ID | [SEQ ID | [SEQ ID | [SEQ ID | | | TGGCTGAGAGAATAGT | UGGCUGAGAGAAUAGU |
| NO: 680] | NO: 1208] | NO: 796] | NO: 1324] | NO: 1028] | | | GAAATATATATTAAAT | GAAAUAUAUAUUAAAU |
| | | | | | | | TCTCTCAGCAGAAGCC | UCUCUCAGCAGAAGCC |
| | | | | | | | TTCTTTAGTGTGACAG | UUCUUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 912] | [SEQ ID NO: 1440] |
| TTAGAAGGCTTT | UUAGAAGGCUUU | TCTCTCAGCCGG | UCUCUCAGCCGG | UCUCUCAGCCAA | -0.48849 | 2941 | ACCGGACATACTTGTT | ACCGGACAUACUUGUU |
| GGCTGAGAGA | GGCUGAGAGA | AGCCTTCTAT | AGCCUUCUAU | AGCCUUCU | | | CCACTCTTAGAAGGCT | CCACUCUUAGAAGGCU |
| [SEQ ID | [SEQ ID | [SEQ ID | [SEQ ID | [SEQ ID | | | TTGGCTGAGAGATAGT | UUGGCUGAGAGAUAGU |
| NO: 681] | NO: 1209] | NO: 797] | NO: 1325] | NO: 1029] | | | GAAATATATATTAAAT | GAAAUAUAUAUUAAAU |
| | (same as | | | | | | CTCTCAGCCGGAGCCT | CUCUCAGCCGGAGCCU |
| | guide in XD- | | | | | | TCTATTAGTGTGACAG | UCUAUUAGUGUGACAG |
| | 14858) | | | | | | GGATACAGCAACTATT | GGAUACAGCAACUAUU |
| | | | | | | | TTATCAA | UUAUCAA |
| | | | | | | | [SEQ ID NO: 913] | [SEQ ID NO: 1441] |
| AGTAGAAGGCTT | AGUAGAAGGCUU | CTCTCAGCCAGG | CUCUCAGCCAGG | CUCUCAGCCAAA | -2.61597 | 2942 | ACCGGACATACTTGTT | ACCGGACAUACUUGUU |
| TGGCTGAGAG | UGGCUGAGAG | GCCTTCTACA | GCCUUCUACA | GCCUUCUA | | | CCACTCAGTAGAAGGC | CCACUCAGUAGAAGGC |
| [SEQ ID | [SEQ ID | [SEQ ID | [SEQ ID | [SEQ ID | | | TTTGGCTGAGAGTAGT | UUUGGCUGAGAGUAGU |
| NO: 682] | NO: 1210] | NO: 798] | NO: 1326] | NO: 1030] | | | GAAATATATATTAAAC | GAAAUAUAUAUUAAAC |
| | | | | | | | TCTCAGCCAGGGCCTT | UCUCAGCCAGGGCCUU |
| | | | | | | | CTACATAGTGTGACAG | CUACAUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 914] | [SEQ ID NO: 1442] |
| TAGTAGAAGGCT | UAGUAGAAGGCU | TCTCAGCCAAGT | UCUCAGCCAAGU | UCUCAGCCAAAG | -2.42796 | 2943 | ACCGGACATACTTGTT | ACCGGACAUACUUGUU |
| TTGGCTGAGA | UUGGCUGAGA | CCTTCTACTT | CCUUCUACUU | CCUUCUAC | | | CCACTCTAGTAGAAGG | CCACUCUAGUAGAAGG |
| [SEQ ID | [SEQ ID | [SEQ ID | [SEQ ID | [SEQ ID | | | CTTTGGCTGAGATAGT | CUUUGGCUGAGAUAGU |
| NO: 683] | NO: 1211] | NO: 799] | NO: 1327] | NO: 1031] | | | GAAATATATATTAAAT | GAAAUAUAUAUUAAAU |
| | | | | | | | CTCAGCCAAGTCCTTC | CUCAGCCAAGUCCUUC |
| | | | | | | | TACTTTAGTGTGACAG | UACUUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 915] | [SEQ ID NO: 1443] |
| TTAGTAGAAGGC | UUAGUAGAAGGC | CTCAGCCAAATA | CUCAGCCAAAUA | CUCAGCCAAAGC | -1.95956 | 2944 | ACCGGACATACTTGTT | ACCGGACAUACUUGUU |
| TTTGGCTGAG | UUUGGCUGAG | CTTCTACTAT | CUUCUACUAU | CUUCUACU | | | CCACTCTTAGTAGAAG | CCACUCUUAGUAGAAG |
| [SEQ ID | [SEQ ID | [SEQ ID | [SEQ ID | [SEQ ID | | | GCTTTGGCTGAGTAGT | GCUUUGGCUGAGUAGU |
| NO: 684] | NO: 1212] | NO: 800] | NO: 1328] | NO: 1032] | | | GAAATATATATTAAAC | GAAAUAUAUAUUAAAC |
| | (same as | | | | | | TCAGCCAAATACTTCT | UCAGCCAAAUACUUCU |
| | guide in XD- | | | | | | ACTATTAGTGTGACAG | ACUAUUAGUGUGACAG |
| | 14859) | | | | | | GGATACAGCAACTATT | GGAUACAGCAACUAUU |
| | | | | | | | TTATCAA | UUAUCAA |
| | | | | | | | [SEQ ID NO: 916] | [SEQ ID NO: 1444] |
| TGTAGTAGAAGG | UGUAGUAGAAGG | TCAGCCAAAGAA | UCAGCCAAAGAA | UCAGCCAAAGCC | -4.21076 | 2945 | ACCGGACATACTTGTT | ACCGGACAUACUUGUU |
| CTTTGGCTGA | CUUUGGCUGA | TTCTACTACT | UUCUACUACU | UUCUACUA | | | CCACTCTGTAGTAGAA | CCACUCUGUAGUAGAA |
| [SEQ ID | [SEQ ID | [SEQ ID | [SEQ ID | [SEQ ID | | | GGCTTTGGCTGATAGT | GGCUUUGGCUGAUAGU |
| NO: 685] | NO: 1213] | NO: 801] | NO: 1329] | NO: 1033] | | | GAAATATATATTAAAT | GAAAUAUAUAUUAAAU |
| | (same as | | | | | | CAGCCAAAGAATTCTA | CAGCCAAAGAAUUCUA |
| | guide in XD- | | | | | | CTACTTAGTGTGACAG | CUACUUAGUGUGACAG |
| | 14860) | | | | | | GGATACAGCAACTATT | GGAUACAGCAACUAUU |
| | | | | | | | TTATCAA | UUAUCAA |
| | | | | | | | [SEQ ID NO: 917] | [SEQ ID NO: 1445] |
| TGGTAGTAGAAG | UGGUAGUAGAAG | CAGCCAAAGCAC | CAGCCAAAGCAC | CAGCCAAAGCCU | -2.92638 | 2946 | ACCGGACATACTTGTT | ACCGGACAUACUUGUU |
| GCTTTGGCTG | GCUUUGGCUG | TCTACTACCT | UCUACUACCU | UCUACUAC | | | CCACTCTGGTAGTAGA | CCACUCUGGUAGUAGA |
| [SEQ ID | [SEQ ID | [SEQ ID | [SEQ ID | [SEQ ID | | | AGGCTTTGGCTGTAGT | AGGCUUUGGCUGUAGU |
| NO: 686] | NO: 1214] | NO: 802] | NO: 1330] | NO: 1034] | | | GAAATATATATTAAAC | GAAAUAUAUAUUAAAC |
| | (same as | | | | | | AGCCAAAGCACTCTAC | AGCCAAAGCACUCUAC |
| | guide in XD- | | | | | | TACCTTAGTGTGACAG | UACCUUAGUGUGACAG |
| | 14861) | | | | | | GGATACAGCAACTATT | GGAUACAGCAACUAUU |
| | | | | | | | TTATCAA | UUAUCAA |
| | | | | | | | [SEQ ID NO: 918] | [SEQ ID NO: 1446] |
| TGAACAAGGGGC | UGAACAAGGGGC | TCCCAAATCATA | UCCCAAAUCAUA | UCCCAAAUCAGC | -4.19616 | 3301 | ACCGGACATACTTGTT | ACCGGACAUACUUGUU |
| TGATTTGGGA | UGAUUUGGGA | CCCTTGTTCT | CCCUUGUUCU | CCCUUGUU | | | CCACTCTGAACAAGGG | CCACUCUGAACAAGGG |
| [SEQ ID | [SEQ ID | [SEQ ID | [SEQ ID | [SEQ ID | | | GCTGATTTGGGATAGT | GCUGAUUUGGGAUAGU |
| NO: 687] | NO: 1215] | NO: 803] | NO: 1331] | NO: 1035] | | | GAAATATATATTAAAT | GAAAUAUAUAUUAAAU |
| | | | | | | | CCCAAATCATACCCTT | CCCAAAUCAUACCCUU |
| | | | | | | | GTTCTTAGTGTGACAG | GUUCUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 919] | [SEQ ID NO: 1447] |
| TTGAACAAGGGG | UUGAACAAGGGG | CCCAAATCAGAA | CCCAAAUCAGAA | CCCAAAUCAGCC | -4.85857 | 3302 | ACCGGACATACTTGTT | ACCGGACAUACUUGUU |
| CTGATTTGGG | CUGAUUUGGG | CCTTGTTCAT | CCUUGUUCAU | CCUUGUUC | | | CCACTCTTGAACAAGG | CCACUCUUGAACAAGG |
| [SEQ ID | [SEQ ID | [SEQ ID | [SEQ ID | [SEQ ID | | | GGCTGATTTGGGTAGT | GGCUGAUUUGGGUAGU |
| NO: 688] | NO: 1216] | NO: 804] | NO: 1332] | NO: 1036] | | | GAAATATATATTAAAC | GAAAUAUAUAUUAAAC |
| | | | | | | | CCAAATCAGAACCTTG | CCAAAUCAGAACCUUG |
| | | | | | | | TTCATTAGTGTGACAG | UUCAUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 920] | [SEQ ID NO: 1448] |
| TCTGAACAAGGG | UCUGAACAAGGG | CCAAATCAGCAA | CCAAAUCAGCAA | CCAAAUCAGCCC | -2.36325 | 3303 | ACCGGACATACTTGTT | ACCGGACAUACUUGUU |
| GCTGATTTGG | GCUGAUUUGG | CTTGTTCAGT | CUUGUUCAGU | CUUGUUCA | | | CCACTCTCTGAACAAG | CCACUCUCUGAACAAG |
| [SEQ ID | [SEQ ID | [SEQ ID | [SEQ ID | [SEQ ID | | | GGGCTGATTTGGTAGT | GGGCUGAUUUGGUAGU |
| NO: 689] | NO: 1217] | NO: 805] | NO: 1333] | NO: 1037] | | | GAAATATATATTAAAC | GAAAUAUAUAUUAAAC |
| | | | | | | | CAAATCAGCAACTTGT | CAAAUCAGCAACUUGU |
| | | | | | | | TCAGTTAGTGTGACAG | UCAGUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 921] | [SEQ ID NO: 1449] |
| TGTGGCACATGC | UGUGGCACAUGC | CCCTTGTTCATA | CCCUUGUUCAUA | CCCUUGUUCAGC | -2.10246 | 3313 | ACCGGACATACTTGTT | ACCGGACAUACUUGUU |
| TGAACAAGGG | UGAACAAGGG | ATGTGCCACT | AUGUGCCACU | AUGUGCCA | | | CCACTCTGTGGCACAT | CCACUCUGUGGCACAU |
| [SEQ ID | [SEQ ID | [SEQ ID | [SEQ ID | [SEQ ID | | | GCTGAACAAGGGTAGT | GCUGAACAAGGGUAGU |
| NO: 690] | NO: 1218] | NO: 806] | NO: 1334] | NO: 1038] | | | GAAATATATATTAAAC | GAAAUAUAUAUUAAAC |
| | | | | | | | CCTTGTTCATAATGTG | CCUUGUUCAUAAUGUG |
| | | | | | | | CCACTTAGTGTGACAG | CCACUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 922] | [SEQ ID NO: 1450] |
| TTGCCATCATTC | UUGCCAUCAUUC | GGTAATGCTATG | GGUAAUGCUAUG | GGUAAUGCUAGA | -0.43513 | 3378 | ACCGGACATACTTGTT | ACCGGACAUACUUGUU |
| TAGCATTACC | UAGCAUUACC | ATGATGGCAT | AUGAUGGCAU | AUGAUGGC | | | CCACTCTTGCCATCAT | CCACUCUUGCCAUCAU |
| [SEQ ID | [SEQ ID | [SEQ ID | [SEQ ID | [SEQ ID | | | TCTAGCATTACCTAGT | UCUAGCAUUACCUAGU |
| NO: 691] | NO: 1219] | NO: 807] | NO: 1335] | NO: 1039] | | | GAAATATATATTAAAG | GAAAUAUAUAUUAAAG |
| | | | | | | | GTAATGCTATGATGAT | GUAAUGCUAUGAUGAU |
| | | | | | | | GGCATTAGTGTGACAG | GGCAUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 923] | [SEQ ID NO: 1451] |
| TTGCTGGGAAAC | UUGCUGGGAAAC | CCACAGAATATC | CCACAGAAUAUC | CCACAGAAUAGU | -2.70652 | 3804 | ACCGGACATACTTGTT | ACCGGACAUACUUGUU |
| TATTCTGTGG | UAUUCUGUGG | TTCCCAGCAT | UUCCCAGCAU | UUCCCAGC | | | CCACTCTTGCTGGGAA | CCACUCUUGCUGGGAA |
| [SEQ ID | [SEQ ID | [SEQ ID | [SEQ ID | [SEQ ID | | | ACTATTCTGTGGTAGT | ACUAUUCUGUGGUAGU |
| NO: 692] | NO: 1220] | NO: 808] | NO: 1336] | NO: 1040] | | | GAAATATATATTAAAC | GAAAUAUAUAUUAAAC |
| | (same as | | | | | | CACAGAATATCTTCCC | CACAGAAUAUCUUCCC |
| | guide in XD- | | | | | | AGCATTAGTGTGACAG | AGCAUUAGUGUGACAG |
| | 14861) | | | | | | GGATACAGCAACTATT | GGAUACAGCAACUAUU |
| | | | | | | | TTATCAA | UUAUCAA |
| | | | | | | | [SEQ ID NO: 924] | [SEQ ID NO: 1452] |
| TCTGCTGGGAAA | UCUGCUGGGAAA | CACAGAATAGCC | CACAGAAUAGCC | CACAGAAUAGUU | -4.91212 | 3805 | ACCGGACATACTTGTT | ACCGGACAUACUUGUU |
| CTATTCTGTG | CUAUUCUGUG | TCCCAGCAGT | UCCCAGCAGU | UCCCAGCA | | | CCACTCTCTGCTGGGA | CCACUCUCUGCUGGGA |
| [SEQ ID | [SEQ ID | [SEQ ID | [SEQ ID | [SEQ ID | | | AACTATTCTGTGTAGT | AACUAUUCUGUGUAGU |
| NO: 693] | NO: 1221] | NO: 809] | NO: 1337] | NO: 1041] | | | GAAATATATATTAAAC | GAAAUAUAUAUUAAAC |
| | | | | | | | ACAGAATAGCCTCCCA | ACAGAAUAGCCUCCCA |
| | | | | | | | GCAGTTAGTGTGACAG | GCAGUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 925] | [SEQ ID NO: 1453] |
| TGCTGCTGGGAA | UGCUGCUGGGAA | ACAGAATAGTCC | ACAGAAUAGUCC | ACAGAAUAGUUU | -3.54976 | 3806 | ACCGGACATACTTGTT | ACCGGACAUACUUGUU |
| ACTATTCTGT | ACUAUUCUGU | CCCAGCAGCT | CCCAGCAGCU | CCCAGCAG | | | CCACTCTGCTGCTGGG | CCACUCUGCUGCUGGG |
| [SEQ ID | [SEQ ID | [SEQ ID | [SEQ ID | [SEQ ID | | | AAACTATTCTGTTAGT | AAACUAUUCUGUUAGU |
| NO: 694] | NO: 1222] | NO: 810] | NO: 1338] | NO: 1042] | | | GAAATATATATTAAAA | GAAAUAUAUAUUAAAA |
| | | | | | | | CAGAATAGTCCCCCAG | CAGAAUAGUCCCCCAG |
| | | | | | | | CAGCTTAGTGTGACAG | CAGCUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 926] | [SEQ ID NO: 1454] |
| TGAACGTGAGAA | UGAACGUGAGAA | CGATCCATCCCC | CGAUCCAUCCCC | CGAUCCAUCCUU | -3.0586 | 3844 | ACCGGACATACTTGTT | ACCGGACAUACUUGUU |
| GGATGGATCG | GGAUGGAUCG | CTCACGTTCT | CUCACGUUCU | CUCACGUU | | | CCACTCTGAACGTGAG | CCACUCUGAACGUGAG |
| [SEQ ID | [SEQ ID | [SEQ ID | [SEQ ID | [SEQ ID | | | AAGGATGGATCGTAGT | AAGGAUGGAUCGUAGU |
| NO: 695] | NO: 1223] | NO: 811] | NO: 1339] | NO: 1043] | | | GAAATATATATTAAAC | GAAAUAUAUAUUAAAC |
| | | | | | | | GATCCATCCCCCTCAC | GAUCCAUCCCCCUCAC |
| | | | | | | | GTTCTTAGTGTGACAG | GUUCUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 927] | [SEQ ID NO: 1455] |
| TTGAACGTGAGA | UUGAACGUGAGA | GATCCATCCTCA | GAUCCAUCCUCA | GAUCCAUCCUUC | -4.40112 | 3845 | ACCGGACATACTTGTT | ACCGGACAUACUUGUU |
| AGGATGGATC | AGGAUGGAUC | TCACGTTCAT | UCACGUUCAU | UCACGUUC | | | CCACTCTTGAACGTGA | CCACUCUUGAACGUGA |
| [SEQ ID | [SEQ ID | [SEQ ID | [SEQ ID | [SEQ ID | | | GAAGGATGGATCTAGT | GAAGGAUGGAUCUAGU |
| NO: 696] | NO: 1224] | NO: 812] | NO: 1340] | NO: 1044] | | | GAAATATATATTAAAG | GAAAUAUAUAUUAAAG |
| | | | | | | | ATCCATCCTCATCACG | AUCCAUCCUCAUCACG |
| | | | | | | | TTCATTAGTGTGACAG | UUCAUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 928] | [SEQ ID NO: 1456] |
| TCTGAACGTGAG | UCUGAACGUGAG | ATCCATCCTTAC | AUCCAUCCUUAC | AUCCAUCCUUCU | -2.54571 | 3846 | ACCGGACATACTTGTT | ACCGGACAUACUUGUU |
| AAGGATGGAT | AAGGAUGGAU | CACGTTCAGT | CACGUUCAGU | CACGUUCA | | | CCACTCTCTGAACGTG | CCACUCUCUGAACGUG |
| [SEQ ID | [SEQ ID | [SEQ ID | [SEQ ID | [SEQ ID | | | AGAAGGATGGATTAGT | AGAAGGAUGGAUUAGU |
| NO: 697] | NO: 1225] | NO: 813] | NO: 1341] | NO: 1045] | | | GAAATATATATTAAAA | GAAAUAUAUAUUAAAA |
| | | | | | | | TCCATCCTTACCACGT | UCCAUCCUUACCACGU |
| | | | | | | | TCAGTTAGTGTGACAG | UCAGUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 929] | [SEQ ID NO: 1457] |
| AACTGTTAGCAT | AACUGUUAGCAU | TCCAATAGGAGC | UCCAAUAGGAGC | UCCAAUAGGAAU | -3.8378 | 4235 | ACCGGACATACTTGTT | ACCGGACAUACUUGUU |
| TCCTATTGGA | UCCUAUUGGA | GCTAACAGTA | GCUAACAGUA | GCUAACAG | | | CCACTCAACTGTTAGC | CCACUCAACUGUUAGC |
| [SEQ ID | [SEQ ID | [SEQ ID | [SEQ ID | [SEQ ID | | | ATTCCTATTGGATAGT | AUUCCUAUUGGAUAGU |
| NO: 698] | NO: 1226] | NO: 814] | NO: 1342] | NO: 1046] | | | GAAATATATATTAAAT | GAAAUAUAUAUUAAAU |
| | | | | | | | CCAATAGGAGCGCTAA | CCAAUAGGAGCGCUAA |
| | | | | | | | CAGTATAGTGTGACAG | CAGUAUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 930] | [SEQ ID NO: 1458] |
| TAACTGTTAGCA | UAACUGUUAGCA | CCAATAGGAACT | CCAAUAGGAACU | CCAAUAGGAAUG | -2.52035 | 4236 | ACCGGACATACTTGTT | ACCGGACAUACUUGUU |
| TTCCTATTGG | UUCCUAUUGG | CTAACAGTTT | CUAACAGUUU | CUAACAGU | | | CCACTCTAACTGTTAG | CCACUCUAACUGUUAG |
| [SEQ ID | [SEQ ID | [SEQ ID | [SEQ ID | [SEQ ID | | | CATTCCTATTGGTAGT | CAUUCCUAUUGGUAGU |
| NO: 699] | NO: 1227] | NO: 815] | NO: 1343] | NO: 1047] | | | GAAATATATATTAAAC | GAAAUAUAUAUUAAAC |
| | | | | | | | CAATAGGAACTCTAAC | CAAUAGGAACUCUAAC |
| | | | | | | | AGTTTTAGTGTGACAG | AGUUUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 931] | [SEQ ID NO: 1459] |
| TGAACTGTTAGC | UGAACUGUUAGC | CAATAGGAATTA | CAAUAGGAAUUA | CAAUAGGAAUGC | -4.90789 | 4237 | ACCGGACATACTTGTT | ACCGGACAUACUUGUU |
| ATTCCTATTG | AUUCCUAUUG | TAACAGTTCT | UAACAGUUCU | UAACAGUU | | | CCACTCTGAACTGTTA | CCACUCUGAACUGUUA |
| [SEQ ID | [SEQ ID | [SEQ ID | [ SEQ ID | [SEQ ID | | | GCATTCCTATTGTAGT | GCAUUCCUAUUGUAGU |
| NO: 700] | NO: 1228] | NO: 816] | NO: 1344] | NO: 1048] | | | GAAATATATATTAAAC | GAAAUAUAUAUUAAAC |
| | | | | | | | AATAGGAATTATAACA | AAUAGGAAUUAUAACA |
| | | | | | | | GTTCTTAGTGTGACAG | GUUCUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 932] | [SEQ ID NO: 1460] |
| TTGAACTGTTAG | UUGAACUGUUAG | AATAGGAATGAC | AAUAGGAAUGAC | AAUAGGAAUGCU | -3.95764 | 4238 | ACCGGACATACTTGTT | ACCGGACAUACUUGUU |
| CATTCCTATT | CAUUCCUAUU | AACAGTTCAT | AACAGUUCAU | AACAGUUC | | | CCACTCTTGAACTGTT | CCACUCUUGAACUGUU |
| [SEQ ID | [ SEQ ID | [SEQ ID | [SEQ ID | [SEQ ID | | | AGCATTCCTATTTAGT | AGCAUUCCUAUUUAGU |
| NO: 701] | NO: 1229] | NO: 817] | NO: 1345] | NO: 1049] | | | GAAATATATATTAAAA | GAAAUAUAUAUUAAAA |
| | | | | | | | ATAGGAATGACAACAG | AUAGGAAUGACAACAG |
| | | | | | | | TTCATTAGTGTGACAG | UUCAUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 933] | [SEQ ID NO: 1461] |
| AGTGAACTGTTA | AGUGAACUGUUA | ATAGGAATGCCG | AUAGGAAUGCCG | AUAGGAAUGCUA | -4.39189 | 4239 | ACCGGACATACTTGTT | ACCGGACAUACUUGUU |
| GCATTCCTAT | GCAUUCCUAU | ACAGTTCACA | ACAGUUCACA | ACAGUUCA | | | CCACTCAGTGAACTGT | CCACUCAGUGAACUGU |
| [SEQ ID | [SEQ ID | [SEQ ID | [SEQ ID | [SEQ ID | | | TAGCATTCCTATTAGT | UAGCAUUCCUAUUAGU |
| NO: 702] | NO: 1230] | NO: 818] | NO: 1346] | NO: 1050] | | | GAAATATATATTAAAA | GAAAUAUAUAUUAAAA |
| | | | | | | | TAGGAATGCCGACAGT | UAGGAAUGCCGACAGU |
| | | | | | | | TCACATAGTGTGACAG | UCACAUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 934] | [SEQ ID NO: 1462] |
| AAGTGAACTGTT | AAGUGAACUGUU | TAGGAATGCTGG | UAGGAAUGCUGG | UAGGAAUGCUAA | -4.80102 | 4240 | ACCGGACATACTTGTT | ACCGGACAUACUUGUU |
| AGCATTCCTA | AGCAUUCCUA | CAGTTCACTA | CAGUUCACUA | CAGUUCAC | | | CCACTCAAGTGAACTG | CCACUCAAGUGAACUG |
| [SEQ ID | [ SEQ ID | [SEQ ID | [SEQ ID | [SEQ ID | | | TTAGCATTCCTATAGT | UUAGCAUUCCUAUAGU |
| NO: 703] | NO: 1231] | NO: 819] | NO: 1347] | NO: 1051] | | | GAAATATATATTAAAT | GAAAUAUAUAUUAAAU |
| | | | | | | | AGGAATGCTGGCAGTT | AGGAAUGCUGGCAGUU |
| | | | | | | | CACTATAGTGTGACAG | CACUAUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 935] | [SEQ ID NO: 1463] |
| TAAGTGAACTGT | UAAGUGAACUGU | AGGAATGCTAGA | AGGAAUGCUAGA | AGGAAUGCUAAC | -2.45702 | 4241 | ACCGGACATACTTGTT | ACCGGACAUACUUGUU |
| TAGCATTCCT | UAGCAUUCCU | AGTTCACTTT | AGUUCACUUU | AGUUCACU | | | CCACTCTAAGTGAACT | CCACUCUAAGUGAACU |
| [SEQ ID | [SEQ ID | [SEQ ID | [SEQ ID | [SEQ ID | | | GTTAGCATTCCTTAGT | GUUAGCAUUCCUUAGU |
| NO: 704] | NO: 1232] | NO: 820] | NO: 1348] | NO: 1052] | | | GAAATATATATTAAAA | GAAAUAUAUAUUAAAA |
| | | | | | | | GGAATGCTAGAAGTTC | GGAAUGCUAGAAGUUC |
| | | | | | | | ACTTTTAGTGTGACAG | ACUUUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 936] | [SEQ ID NO: 1464] |
| TCAAGTGAACTG | UCAAGUGAACUG | GGAATGCTAAAG | GGAAUGCUAAAG | GGAAUGCUAACA | -4.855 | 4242 | ACCGGACATACTTGTT | ACCGGACAUACUUGUU |
| TTAGCATTCC | UUAGCAUUCC | GTTCACTTGT | GUUCACUUGU | GUUCACUU | | | CCACTCTCAAGTGAAC | CCACUCUCAAGUGAAC |
| [SEQ ID | [SEQ ID | [SEQ ID | [SEQ ID | [SEQ ID | | | TGTTAGCATTCCTAGT | UGUUAGCAUUCCUAGU |
| NO: 705] | NO: 1233] | NO: 821] | NO: 1349] | NO: 1053] | | | GAAATATATATTAAAG | GAAAUAUAUAUUAAAG |
| | | | | | | | GAATGCTAAAGGTTCA | GAAUGCUAAAGGUUCA |
| | | | | | | | CTTGTTAGTGTGACAG | CUUGUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 937] | [SEQ ID NO: 1465] |
| TGCAAGTGAACT | UGCAAGUGAACU | GAATGCTAACGT | GAAUGCUAACGU | GAAUGCUAACAG | -4.05556 | 4243 | ACCGGACATACTTGTT | ACCGGACAUACUUGUU |
| GTTAGCATTC | GUUAGCAUUC | TTCACTTGCT | UUCACUUGCU | UUCACUUG | | | CCACTCTGCAAGTGAA | CCACUCUGCAAGUGAA |
| [SEQ ID | [SEQ ID | [SEQ ID | [SEQ ID | [SEQ ID | | | CTGTTAGCATTCTAGT | CUGUUAGCAUUCUAGU |
| NO: 706] | NO: 1234] | NO: 822] | NO: 1350] | NO: 1054] | | | GAAATATATATTAAAG | GAAAUAUAUAUUAAAG |
| | | | | | | | AATGCTAACGTTTCAC | AAUGCUAACGUUUCAC |
| | | | | | | | TTGCTTAGTGTGACAG | UUGCUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 938] | [SEQ ID NO: 1466] |
| TTGCAAGTGAAC | UUGCAAGUGAAC | AATGCTAACATC | AAUGCUAACAUC | AAUGCUAACAGU | -3.92834 | 4244 | ACCGGACATACTTGTT | ACCGGACAUACUUGUU |
| TGTTAGCATT | UGUUAGCAUU | TCACTTGCAT | UCACUUGCAU | UCACUUGC | | | CCACTCTTGCAAGTGA | CCACUCUUGCAAGUGA |
| [SEQ ID | [SEQ ID | [SEQ ID | [SEQ ID | [SEQ ID | | | ACTGTTAGCATTTAGT | ACUGUUAGCAUUUAGU |
| NO: 707] | NO: 1235] | NO: 823] | NO: 1351] | NO: 1055] | | | GAAATATATATTAAAA | GAAAUAUAUAUUAAAA |
| | | | | | | | ATGCTAACATCTCACT | AUGCUAACAUCUCACU |
| | | | | | | | TGCATTAGTGTGACAG | UGCAUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 939] | [SEQ ID NO: 1467] |
| ACTGCAAGTGAA | ACUGCAAGUGAA | ATGCTAACAGCC | AUGCUAACAGCC | AUGCUAACAGUU | -4.32689 | 4245 | ACCGGACATACTTGTT | ACCGGACAUACUUGUU |
| CTGTTAGCAT | CUGUUAGCAU | CACTTGCAGA | CACUUGCAGA | CACUUGCA | | | CCACTCACTGCAAGTG | CCACUCACUGCAAGUG |
| [SEQ ID | [SEQ ID | [SEQ ID | [SEQ ID | [SEQ ID | | | AACTGTTAGCATTAGT | AACUGUUAGCAUUAGU |
| NO: 708] | NO: 1236] | NO: 824] | NO: 1352] | NO: 1056] | | | GAAATATATATTAAAA | GAAAUAUAUAUUAAAA |
| | | | | | | | TGCTAACAGCCCACTT | UGCUAACAGCCCACUU |
| | | | | | | | GCAGATAGTGTGACAG | GCAGAUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 940] | [SEQ ID NO: 1468] |
| TACTGCAAGTGA | UACUGCAAGUGA | TGCTAACAGTCA | UGCUAACAGUCA | UGCUAACAGUUC | -4.77129 | 4246 | ACCGGACATACTTGTT | ACCGGACAUACUUGUU |
| ACTGTTAGCA | ACUGUUAGCA | ACTTGCAGTT | ACUUGCAGUU | ACUUGCAG | | | CCACTCTACTGCAAGT | CCACUCUACUGCAAGU |
| [SEQ ID | [SEQ ID | [SEQ ID | [SEQ ID | [SEQ ID | | | GAACTGTTAGCATAGT | GAACUGUUAGCAUAGU |
| NO: 709] | NO: 1237] | NO: 825] | NO: 1353] | NO: 1057] | | | GAAATATATATTAAAT | GAAAUAUAUAUUAAAU |
| | | | | | | | GCTAACAGTCAACTTG | GCUAACAGUCAACUUG |
| | | | | | | | CAGTTTAGTGTGACAG | CAGUUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 941] | [SEQ ID NO: 1469] |
| TCACTGCAAGTG | UCACUGCAAGUG | GCTAACAGTTAG | GCUAACAGUUAG | GCUAACAGUUCA | -3.83727 | 4247 | ACCGGACATACTTGTT | ACCGGACAUACUUGUU |
| AACTGTTAGC | AACUGUUAGC | CTTGCAGTGT | CUUGCAGUGU | CUUGCAGU | | | CCACTCTCACTGCAAG | CCACUCUCACUGCAAG |
| [SEQ ID | [SEQ ID | [SEQ ID | [SEQ ID | [SEQ ID | | | TGAACTGTTAGCTAGT | UGAACUGUUAGCUAGU |
| NO: 710] | NO: 1238] | NO: 826] | NO: 1354] | NO: 1058] | | | GAAATATATATTAAAG | GAAAUAUAUAUUAAAG |
| | | | | | | | CTAACAGTTAGCTTGC | CUAACAGUUAGCUUGC |
| | | | | | | | AGTGTTAGTGTGACAG | AGUGUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 942 | [SEQ ID NO: 1470] |
| TCCACTGCAAGT | UCCACUGCAAGU | CTAACAGTTCGA | CUAACAGUUCGA | CUAACAGUUCAC | -4.9039 | 4248 | ACCGGACATACTTGTT | ACCGGACAUACUUGUU |
| GAACTGTTAG | GAACUGUUAG | TTGCAGTGGT | UUGCAGUGGU | UUGCAGUG | | | CCACTCTCCACTGCAA | CCACUCUCCACUGCAA |
| [SEQ ID | [SEQ ID | [SEQ ID | [SEQ ID | [SEQ ID | | | GTGAACTGTTAGTAGT | GUGAACUGUUAGUAGU |
| NO: 711] | NO: 1239] | NO: 827] | NO: 1355] | NO: 1059] | | | GAAATATATATTAAAC | GAAAUAUAUAUUAAAC |
| | | | | | | | TAACAGTTCGATTGCA | UAACAGUUCGAUUGCA |
| | | | | | | | GTGGTTAGTGTGACAG | GUGGUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 943] | [SEQ ID NO: 1471] |
| TTCCACTGCAAG | UUCCACUGCAAG | TAACAGTTCAAC | UAACAGUUCAAC | UAACAGUUCACU | -3.84653 | 4249 | ACCGGACATACTTGTT | ACCGGACAUACUUGUU |
| TGAACTGTTA | UGAACUGUUA | TGCAGTGGAT | UGCAGUGGAU | UGCAGUGG | | | CCACTCTTCCACTGCA | CCACUCUUCCACUGCA |
| [SEQ ID | [SEQ ID | [SEQ ID | [SEQ ID | [SEQ ID | | | AGTGAACTGTTATAGT | AGUGAACUGUUAUAGU |
| NO: 712] | NO: 1240] | NO: 828] | NO: 1356] | NO: 1060] | | | GAAATATATATTAAAT | GAAAUAUAUAUUAAAU |
| | | | | | | | AACAGTTCAACTGCAG | AACAGUUCAACUGCAG |
| | | | | | | | TGGATTAGTGTGACAG | UGGAUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 944] | [SEQ ID NO: 1472] |
| TTTCCACTGCAA | UUUCCACUGCAA | AACAGTTCACCC | AACAGUUCACCC | AACAGUUCACUU | -3.95941 | 4250 | ACCGGACATACTTGTT | ACCGGACAUACUUGUU |
| GTGAACTGTT | GUGAACUGUU | GCAGTGGAAT | GCAGUGGAAU | GCAGUGGA | | | CCACTCTTTCCACTGC | CCACUCUUUCCACUGC |
| [SEQ ID | [SEQ ID | [SEQ ID | [SEQ ID | [SEQ ID | | | AAGTGAACTGTTTAGT | AAGUGAACUGUUUAGU |
| NO: 713] | NO: 1241] | NO: 829] | NO: 1357] | NO: 1061] | | | GAAATATATATTAAAA | GAAAUAUAUAUUAAAA |
| | | | | | | | ACAGTTCACCCGCAGT | ACAGUUCACCCGCAGU |
| | | | | | | | GGAATTAGTGTGACAG | GGAAUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 945] | [SEQ ID NO: 1473] |
| TCTTCCACTGCA | UCUUCCACUGCA | ACAGTTCACTCT | ACAGUUCACUCU | ACAGUUCACUUG | -4.68439 | 4251 | ACCGGACATACTTGTT | ACCGGACAUACUUGUU |
| AGTGAACTGT | AGUGAACUGU | CAGTGGAAGT | CAGUGGAAGU | CAGUGGAA | | | CCACTCTCTTCCACTG | CCACUCUCUUCCACUG |
| [SEQ ID | [SEQ ID | [SEQ ID | [SEQ ID | [SEQ ID | | | CAAGTGAACTGTTAGT | CAAGUGAACUGUUAGU |
| NO: 714] | NO: 1242] | NO: 830] | NO: 1358] | NO: 1062] | | | GAAATATATATTAAAA | GAAAUAUAUAUUAAAA |
| | | | | | | | CAGTTCACTCTCAGTG | CAGUUCACUCUCAGUG |
| | | | | | | | GAAGTTAGTGTGACAG | GAAGUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 946] | [SEQ ID NO: 1474] |
| ATCTTCCACTGC | AUCUUCCACUGC | CAGTTCACTTTA | CAGUUCACUUUA | CAGUUCACUUGC | -2.98094 | 4252 | ACCGGACATACTTGTT | ACCGGACAUACUUGUU |
| AAGTGAACTG | AAGUGAACUG | AGTGGAAGAA | AGUGGAAGAA | AGUGGAAG | | | CCACTCATCTTCCACT | CCACUCAUCUUCCACU |
| [SEQ ID | [SEQ ID | [SEQ ID | [SEQ ID | [SEQ ID | | | GCAAGTGAACTGTAGT | GCAAGUGAACUGUAGU |
| NO: 715] | NO: 1243] | NO: 831] | NO: 1359] | NO: 1063] | | | GAAATATATATTAAAC | GAAAUAUAUAUUAAAC |
| | | | | | | | AGTTCACTTTAAGTGG | AGUUCACUUUAAGUGG |
| | | | | | | | AAGAATAGTGTGACAG | AAGAAUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 947] | [SEQ ID NO: 1475] |
| TATCTTCCACTG | UAUCUUCCACUG | AGTTCACTTGAG | AGUUCACUUGAG | AGUUCACUUGCA | -3.46197 | 4253 | ACCGGACATACTTGTT | ACCGGACAUACUUGUU |
| CAAGTGAACT | CAAGUGAACU | GTGGAAGATT | GUGGAAGAUU | GUGGAAGA | | | CCACTCTATCTTCCAC | CCACUCUAUCUUCCAC |
| [SEQ ID | [SEQ ID | [SEQ ID | [SEQ ID | [SEQ ID | | | TGCAAGTGAACTTAGT | UGCAAGUGAACUUAGU |
| NO: 716] | NO: 1244] | NO: 832] | NO: 1360] | NO: 1064] | | | GAAATATATATTAAAA | GAAAUAUAUAUUAAAA |
| | | | | | | | GTTCACTTGAGGTGGA | GUUCACUUGAGGUGGA |
| | | | | | | | AGATTTAGTGTGACAG | AGAUUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 948] | [SEQ ID NO: 1476] |
| TGGCAAGCAGAG | UGGCAAGCAGAG | GGTACCCCAGAC | GGUACCCCAGAC | GGUACCCCAGCU | -3.53049 | 4348 | ACCGGACATACTTGTT | ACCGGACAUACUUGUU |
| CTGGGGTACC | CUGGGGUACC | CTGCTTGCCT | CUGCUUGCCU | CUGCUUGC | | | CCACTCTGGCAAGCAG | CCACUCUGGCAAGCAG |
| [SEQ ID | [SEQ ID | [SEQ ID | [SEQ ID | [SEQ ID | | | AGCTGGGGTACCTAGT | AGCUGGGGUACCUAGU |
| NO: 717] | NO: 1245] | NO: 833] | NO: 1361] | NO: 1065] | | | GAAATATATATTAAAG | GAAAUAUAUAUUAAAG |
| | | | | | | | GTACCCCAGACCTGCT | GUACCCCAGACCUGCU |
| | | | | | | | TGCCTTAGTGTGACAG | UGCCUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 949] | [SEQ ID NO: 1477] |
| TCGGCAAGCAGA | UCGGCAAGCAGA | GTACCCCAGCCA | GUACCCCAGCCA | GUACCCCAGCUC | -4.36361 | 4349 | ACCGGACATACTTGTT | ACCGGACAUACUUGUU |
| GCTGGGGTAC | GCUGGGGUAC | TGCTTGCCGT | UGCUUGCCGU | UGCUUGCC | | | CCACTCTCGGCAAGCA | CCACUCUCGGCAAGCA |
| [SEQ ID | [SEQ ID | [SEQ ID | [SEQ ID | [SEQ ID | | | GAGCTGGGGTACTAGT | GAGCUGGGGUACUAGU |
| NO: 718] | NO: 1246] | NO: 834] | NO: 1362] | NO: 1066] | | | GAAATATATATTAAAG | GAAAUAUAUAUUAAAG |
| | | | | | | | TACCCCAGCCATGCTT | UACCCCAGCCAUGCUU |
| | | | | | | | GCCGTTAGTGTGACAG | GCCGUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 950] | [SEQ ID NO: 1478] |
| TTCGGCAAGCAG | UUCGGCAAGCAG | TACCCCAGCTAC | UACCCCAGCUAC | UACCCCAGCUCU | -4.40568 | 4350 | ACCGGACATACTTGTT | ACCGGACAUACUUGUU |
| AGCTGGGGTA | AGCUGGGGUA | GCTTGCCGAT | GCUUGCCGAU | GCUUGCCG | | | CCACTCTTCGGCAAGC | CCACUCUUCGGCAAGC |
| [SEQ ID | [SEQ ID | [SEQ ID | [SEQ ID | [SEQ ID | | | AGAGCTGGGGTATAGT | AGAGCUGGGGUAUAGU |
| NO: 719] | NO: 1247] | NO: 835] | NO: 1363] | NO: 1067] | | | GAAATATATATTAAAT | GAAAUAUAUAUUAAAU |
| | | | | | | | ACCCCAGCTACGCTTG | ACCCCAGCUACGCUUG |
| | | | | | | | CCGATTAGTGTGACAG | CCGAUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 951] | [SEQ ID NO: 1479] |
| TTTCGGCAAGCA | UUUCGGCAAGCA | ACCCCAGCTCCT | ACCCCAGCUCCU | ACCCCAGCUCUG | -3.51989 | 4351 | ACCGGACATACTTGTT | ACCGGACAUACUUGUU |
| GAGCTGGGGT | GAGCUGGGGU | CTTGCCGAAT | CUUGCCGAAU | CUUGCCGA | | | CCACTCTTTCGGCAAG | CCACUCUUUCGGCAAG |
| [SEQ ID | [SEQ ID | [SEQ ID | [SEQ ID | [SEQ ID | | | CAGAGCTGGGGTTAGT | CAGAGCUGGGGUUAGU |
| NO: 720] | NO: 1248] | NO: 836] | NO: 1364] | NO: 1068] | | | GAAATATATATTAAAA | GAAAUAUAUAUUAAAA |
| | | | | | | | CCCCAGCTCCTCTTGC | CCCCAGCUCCUCUUGC |
| | | | | | | | CGAATTAGTGTGACAG | CGAAUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 952] | [SEQ ID NO: 1480] |
| TTTTCGGCAAGC | UUUUCGGCAAGC | CCCCAGCTCTTA | CCCCAGCUCUUA | CCCCAGCUCUGC | -2.43883 | 4352 | ACCGGACATACTTGTT | ACCGGACAUACUUGUU |
| AGAGCTGGGG | AGAGCUGGGG | TTGCCGAAAT | UUGCCGAAAU | UUGCCGAA | | | CCACTCTTTTCGGCAA | CCACUCUUUUCGGCAA |
| [SEQ ID | [SEQ ID | [SEQ ID | [SEQ ID | [SEQ ID | | | GCAGAGCTGGGGTAGT | GCAGAGCUGGGGUAGU |
| NO: 721] | NO: 1249] | NO: 837] | NO: 1365] | NO: 1069] | | | GAAATATATATTAAAC | GAAAUAUAUAUUAAAC |
| | | | | | | | CCCAGCTCTTATTGCC | CCCAGCUCUUAUUGCC |
| | | | | | | | GAAATTAGTGTGACAG | GAAAUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 953] | [SEQ ID NO: 1481] |
| AGTTTCGGCAAG | AGUUUCGGCAAG | CCCAGCTCTGAC | CCCAGCUCUGAC | CCCAGCUCUGCU | -3.70458 | 4353 | ACCGGACATACTTGTT | ACCGGACAUACUUGUU |
| CAGAGCTGGG | CAGAGCUGGG | TGCCGAAACA | UGCCGAAACA | UGCCGAAA | | | CCACTCAGTTTCGGCA | CCACUCAGUUUCGGCA |
| [SEQ ID | [SEQ ID | [SEQ ID | [SEQ ID | [SEQ ID | | | AGCAGAGCTGGGTAGT | AGCAGAGCUGGGUAGU |
| NO: 722] | NO: 1250] | NO: 838] | NO: 1366] | NO: 1070] | | | GAAATATATATTAAAC | GAAAUAUAUAUUAAAC |
| | | | | | | | CCAGCTCTGACTGCCG | CCAGCUCUGACUGCCG |
| | | | | | | | AAACATAGTGTGACAG | AAACAUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 954] | [SEQ ID NO: 1482] |
| TAGTTTCGGCAA | UAGUUUCGGCAA | CCAGCTCTGCCC | CCAGCUCUGCCC | CCAGCUCUGCUU | -2.834 | 4354 | ACCGGACATACTTGTT | ACCGGACAUACUUGUU |
| GCAGAGCTGG | GCAGAGCUGG | GCCGAAACTT | GCCGAAACUU | GCCGAAAC | | | CCACTCTAGTTTCGGC | CCACUCUAGUUUCGGC |
| [SEQ ID | [SEQ ID | [SEQ ID | [SEQ ID | [SEQ ID | | | AAGCAGAGCTGGTAGT | AAGCAGAGCUGGUAGU |
| NO: 723] | NO: 1251] | NO: 839] | NO: 1367] | NO: 1071] | | | GAAATATATATTAAAC | GAAAUAUAUAUUAAAC |
| | | | | | | | CAGCTCTGCCCGCCGA | CAGCUCUGCCCGCCGA |
| | | | | | | | AACTTTAGTGTGACAG | AACUUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 955] | [SEQ ID NO: 1483] |
| TCAGTTTCGGCA | UCAGUUUCGGCA | CAGCTCTGCTCT | CAGCUCUGCUCU | CAGCUCUGCUUG | -4.92603 | 4355 | ACCGGACATACTTGTT | ACCGGACAUACUUGUU |
| AGCAGAGCTG | AGCAGAGCUG | CCGAAACTGT | CCGAAACUGU | CCGAAACU | | | CCACTCTCAGTTTCGG | CCACUCUCAGUUUCGG |
| [SEQ ID | [SEQ ID | [SEQ ID | [SEQ ID | [SEQ ID | | | CAAGCAGAGCTGTAGT | CAAGCAGAGCUGUAGU |
| NO: 724] | NO: 1252] | NO: 840] | NO: 1368] | NO: 1072] | | | GAAATATATATTAAAC | GAAAUAUAUAUUAAAC |
| | | | | | | | AGCTCTGCTCTCCGAA | AGCUCUGCUCUCCGAA |
| | | | | | | | ACTGTTAGTGTGACAG | ACUGUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 956] | [SEQ ID NO: 1484] |
| TCCAGTTTCGGC | UCCAGUUUCGGC | AGCTCTGCTTTA | AGCUCUGCUUUA | AGCUCUGCUUGC | -4.91921 | 4356 | ACCGGACATACTTGTT | ACCGGACAUACUUGUU |
| AAGCAGAGCT | AAGCAGAGCU | CGAAACTGGT | CGAAACUGGU | CGAAACUG | | | CCACTCTCCAGTTTCG | CCACUCUCCAGUUUCG |
| [SEQ ID | [SEQ ID | [SEQ ID | [SEQ ID | [SEQ ID | | | GCAAGCAGAGCTTAGT | GCAAGCAGAGCUUAGU |
| NO: 725] | NO: 1253] | NO: 841] | NO: 1369] | NO: 1073] | | | GAAATATATATTAAAA | GAAAUAUAUAUUAAAA |
| | (same as | | | | | | GCTCTGCTTTACGAAA | GCUCUGCUUUACGAAA |
| | guide in XD- | | | | | | CTGGTTAGTGTGACAG | CUGGUUAGUGUGACAG |
| | 14861) | | | | | | GGATACAGCAACTATT | GGAUACAGCAACUAUU |
| | | | | | | | TTATCAA | UUAUCAA |
| | | | | | | | [SEQ ID NO: 957] | [SEQ ID NO: 1485] |
| TTCCAGTTTCGG | UUCCAGUUUCGG | GCTCTGCTTGAA | GCUCUGCUUGAA | GCUCUGCUUGCC | -4.12374 | 4357 | ACCGGACATACTTGTT | ACCGGACAUACUUGUU |
| CAAGCAGAGC | CAAGCAGAGC | GAAACTGGAT | GAAACUGGAU | GAAACUGG | | | CCACTCTTCCAGTTTC | CCACUCUUCCAGUUUC |
| [SEQ ID | [SEQ ID | [SEQ ID | [SEQ ID | [SEQ ID | | | GGCAAGCAGAGCTAGT | GGCAAGCAGAGCUAGU |
| NO: 726] | NO: 1254] | NO: 842] | NO: 1370] | NO: 1074] | | | GAAATATATATTAAAG | GAAAUAUAUAUUAAAG |
| | | | | | | | CTCTGCTTGAAGAAAC | CUCUGCUUGAAGAAAC |
| | | | | | | | TGGATTAGTGTGACAG | UGGAUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 958] | [SEQ ID NO: 1486] |
| TTTCCAGTTTCG | UUUCCAGUUUCG | CTCTGCTTGCAT | CUCUGCUUGCAU | CUCUGCUUGCCG | -4.13551 | 4358 | ACCGGACATACTTGTT | ACCGGACAUACUUGUU |
| GCAAGCAGAG | GCAAGCAGAG | AAACTGGAAT | AAACUGGAAU | AAACUGGA | | | CCACTCTTTCCAGTTT | CCACUCUUUCCAGUUU |
| [SEQ ID | [SEQ ID | [SEQ ID | [SEQ ID | [SEQ ID | | | CGGCAAGCAGAGTAGT | CGGCAAGCAGAGUAGU |
| NO: 727] | NO: 1255] | NO: 843] | NO: 1371] | NO: 1075] | | | GAAATATATATTAAAC | GAAAUAUAUAUUAAAC |
| | | | | | | | TCTGCTTGCATAAACT | UCUGCUUGCAUAAACU |
| | | | | | | | GGAATTAGTGTGACAG | GGAAUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 959] | [SEQ ID NO: 1487] |
| ACTTCCAGTTTC | ACUUCCAGUUUC | TCTGCTTGCCTG | UCUGCUUGCCUG | UCUGCUUGCCGA | -4.43988 | 4359 | ACCGGACATACTTGTT | ACCGGACAUACUUGUU |
| GGCAAGCAGA | GGCAAGCAGA | AACTGGAAGA | AACUGGAAGA | AACUGGAA | | | CCACTCACTTCCAGTT | CCACUCACUUCCAGUU |
| [SEQ ID | [SEQ ID | [SEQ ID | [SEQ ID | [SEQ ID | | | TCGGCAAGCAGATAGT | UCGGCAAGCAGAUAGU |
| NO: 728] | NO: 1256] | NO: 844] | NO: 1372] | NO: 1076] | | | GAAATATATATTAAAT | GAAAUAUAUAUUAAAU |
| | | | | | | | CTGCTTGCCTGAACTG | CUGCUUGCCUGAACUG |
| | | | | | | | GAAGATAGTGTGACAG | GAAGAUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 960] | [SEQ ID NO: 1488] |
| AACTTCCAGTTT | AACUUCCAGUUU | CTGCTTGCCGGG | CUGCUUGCCGGG | CUGCUUGCCGAA | -3.57411 | 4360 | ACCGGACATACTTGTT | ACCGGACAUACUUGUU |
| CGGCAAGCAG | CGGCAAGCAG | ACTGGAAGTA | ACUGGAAGUA | ACUGGAAG | | | CCACTCAACTTCCAGT | CCACUCAACUUCCAGU |
| [SEQ ID | [SEQ ID | [SEQ ID | [SEQ ID | [SEQ ID | | | TTCGGCAAGCAGTAGT | UUCGGCAAGCAGUAGU |
| NO: 729] | NO: 1257] | NO: 845] | NO: 1373] | NO: 1077] | | | GAAATATATATTAAAC | GAAAUAUAUAUUAAAC |
| | | | | | | | TGCTTGCCGGGACTGG | UGCUUGCCGGGACUGG |
| | | | | | | | AAGTATAGTGTGACAG | AAGUAUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 961] | [SEQ ID NO: 1489] |
| TAACTTCCAGTT | UAACUUCCAGUU | TGCTTGCCGAGG | UGCUUGCCGAGG | UGCUUGCCGAAA | -3.33142 | 4361 | ACCGGACATACTTGTT | ACCGGACAUACUUGUU |
| TCGGCAAGCA | UCGGCAAGCA | CTGGAAGTTT | CUGGAAGUUU | CUGGAAGU | | | CCACTCTAACTTCCAG | CCACUCUAACUUCCAG |
| [SEQ ID | [SEQ ID | [SEQ ID | [SEQ ID | [SEQ ID | | | TTTCGGCAAGCATAGT | UUUCGGCAAGCAUAGU |
| NO: 730] | NO: 1258] | NO: 846] | NO: 1374] | NO: 1078] | | | GAAATATATATTAAAT | GAAAUAUAUAUUAAAU |
| | | | | | | | GCTTGCCGAGGCTGGA | GCUUGCCGAGGCUGGA |
| | | | | | | | AGTTTTAGTGTGACAG | AGUUUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 962] | [SEQ ID NO: 1490] |
| ATAACTTCCAGT | AUAACUUCCAGU | GCTTGCCGAAGA | GCUUGCCGAAGA | GCUUGCCGAAAC | -3.93535 | 4362 | ACCGGACATACTTGTT | ACCGGACAUACUUGUU |
| TTCGGCAAGC | UUCGGCAAGC | TGGAAGTTAA | UGGAAGUUAA | UGGAAGUU | | | CCACTCATAACTTCCA | CCACUCAUAACUUCCA |
| [SEQ ID | [SEQ ID | [SEQ ID | [SEQ ID | [SEQ ID | | | GTTTCGGCAAGCTAGT | GUUUCGGCAAGCUAGU |
| NO: 731] | NO: 1259] | NO: 847] | NO: 1375] | NO: 1079] | | | GAAATATATATTAAAG | GAAAUAUAUAUUAAAG |
| | (same as | | | | | | CTTGCCGAAGATGGAA | CUUGCCGAAGAUGGAA |
| | guide in XD- | | | | | | GTTAATAGTGTGACAG | GUUAAUAGUGUGACAG |
| | 14932) | | | | | | GGATACAGCAACTATT | GGAUACAGCAACUAUU |
| | | | | | | | TTATCAA | UUAUCAA |
| | | | | | | | [SEQ ID NO: 963] | [SEQ ID NO: 1491] |
| AATAACTTCCAG | AAUAACUUCCAG | CTTGCCGAAAAC | CUUGCCGAAAAC | CUUGCCGAAACU | -3.56666 | 4363 | ACCGGACATACTTGTT | ACCGGACAUACUUGUU |
| TTTCGGCAAG | UUUCGGCAAG | GGAAGTTATA | GGAAGUUAUA | GGAAGUUA | | | CCACTCAATAACTTCC | CCACUCAAUAACUUCC |
| [SEQ ID | [SEQ ID | [SEQ ID | [SEQ ID | [SEQ ID | | | AGTTTCGGCAAGTAGT | AGUUUCGGCAAGUAGU |
| NO: 732] | NO: 1260] | NO: 848] | NO: 1376] | NO: 1080] | | | GAAATATATATTAAAC | GAAAUAUAUAUUAAAC |
| | | | | | | | TTGCCGAAAACGGAAG | UUGCCGAAAACGGAAG |
| | | | | | | | TTATATAGTGTGACAG | UUAUAUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 964] | [SEQ ID NO: 1492] |
| AAATAACTTCCA | AAAUAACUUCCA | TTGCCGAAACCT | UUGCCGAAACCU | UUGCCGAAACUG | -2.9553 | 4364 | ACCGGACATACTTGTT | ACCGGACAUACUUGUU |
| GTTTCGGCAA | GUUUCGGCAA | GAAGTTATTA | GAAGUUAUUA | GAAGUUAU | | | CCACTCAAATAACTTC | CCACUCAAAUAACUUC |
| [SEQ ID | [SEQ ID | [SEQ ID | [SEQ ID | [SEQ ID | | | CAGTTTCGGCAATAGT | CAGUUUCGGCAAUAGU |
| NO: 733] | NO: 1261] | NO: 849] | NO: 1377] | NO: 1081] | | | GAAATATATATTAAAT | GAAAUAUAUAUUAAAU |
| | | | | | | | TGCCGAAACCTGAAGT | UGCCGAAACCUGAAGU |
| | | | | | | | TATTATAGTGTGACAG | UAUUAUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 965] | [SEQ ID NO: 1493] |
| ATAAATAACTTC | AUAAAUAACUUC | GCCGAAACTGTG | GCCGAAACUGUG | GCCGAAACUGGA | -3.3862 | 4366 | ACCGGACATACTTGTT | ACCGGACAUACUUGUU |
| CAGTTTCGGC | CAGUUUCGGC | AGTTATTTAA | AGUUAUUUAA | AGUUAUUU | | | CCACTCATAAATAACT | CCACUCAUAAAUAACU |
| [SEQ ID | [SEQ ID | [SEQ ID | [SEQ ID | [SEQ ID | | | TCCAGTTTCGGCTAGT | UCCAGUUUCGGCUAGU |
| NO: 734] | NO: 1262] | NO: 850] | NO: 1378] | NO: 1082] | | | GAAATATATATTAAAG | GAAAUAUAUAUUAAAG |
| | | | | | | | CCGAAACTGTGAGTTA | CCGAAACUGUGAGUUA |
| | | | | | | | TTTAATAGTGTGACAG | UUUAAUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 966] | [SEQ ID NO: 1494] |
| AATAAATAACTT | AAUAAAUAACUU | CCGAAACTGGGG | CCGAAACUGGGG | CCGAAACUGGAA | -3.45626 | 4367 | ACCGGACATACTTGTT | ACCGGACAUACUUGUU |
| CCAGTTTCGG | CCAGUUUCGG | GTTATTTATA | GUUAUUUAUA | GUUAUUUA | | | CCACTCAATAAATAAC | CCACUCAAUAAAUAAC |
| [SEQ ID | [SEQ ID | [SEQ ID | [SEQ ID | [SEQ ID | | | TTCCAGTTTCGGTAGT | UUCCAGUUUCGGUAGU |
| NO: 735] | NO: 1263] | NO: 851] | NO: 1379] | NO: 1083] | | | GAAATATATATTAAAC | GAAAUAUAUAUUAAAC |
| | | | | | | | CGAAACTGGGGGTTAT | CGAAACUGGGGGUUAU |
| | | | | | | | TTATATAGTGTGACAG | UUAUAUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 967] | [SEQ ID NO: 1495] |
| AAATAAATAACT | AAAUAAAUAACU | CGAAACTGGAGT | CGAAACUGGAGU | CGAAACUGGAAG | -1.76663 | 4368 | ACCGGACATACTTGTT | ACCGGACAUACUUGUU |
| TCCAGTTTCG | UCCAGUUUCG | TTATTTATTA | UUAUUUAUUA | UUAUUUAU | | | CCACTCAAATAAATAA | CCACUCAAAUAAAUAA |
| [SEQ ID | [SEQ ID | [SEQ ID | [SEQ ID | [SEQ ID | | | CTTCCAGTTTCGTAGT | CUUCCAGUUUCGUAGU |
| NO: 736] | NO: 1264] | NO: 852] | NO: 1380] | NO: 1084] | | | GAAATATATATTAAAC | GAAAUAUAUAUUAAAC |
| | | | | | | | GAAACTGGAGTTTATT | GAAACUGGAGUUUAUU |
| | | | | | | | TATTATAGTGTGACAG | UAUUAUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 968] | [SEQ ID NO: 1496] |
| AAAATAAATAAC | AAAAUAAAUAAC | GAAACTGGAATC | GAAACUGGAAUC | GAAACUGGAAGU | -0.22922 | 4369 | ACCGGACATACTTGTT | ACCGGACAUACUUGUU |
| TTCCAGTTTC | UUCCAGUUUC | TATTTATTTA | UAUUUAUUUA | UAUUUAUU | | | CCACTCAAAATAAATA | CCACUCAAAAUAAAUA |
| [SEQ ID | [SEQ ID | [SEQ ID | [SEQ ID | [SEQ ID | | | ACTTCCAGTTTCTAGT | ACUUCCAGUUUCUAGU |
| NO: 737] | NO: 1265] | NO: 853] | NO: 1381] | NO: 1085] | | | GAAATATATATTAAAG | GAAAUAUAUAUUAAAG |
| | | | | | | | AAACTGGAATCTATTT | AAACUGGAAUCUAUUU |
| | | | | | | | ATTTATAGTGTGACAG | AUUUAUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 969] | [SEQ ID NO: 1497] |
| AAAAATAAATAA | AAAAAUAAAUAA | AAACTGGAAGCC | AAACUGGAAGCC | AAACUGGAAGUU | -0.00312 | 4370 | ACCGGACATACTTGTT | ACCGGACAUACUUGUU |
| CTTCCAGTTT | CUUCCAGUUU | ATTTATTTTA | AUUUAUUUUA | AUUUAUUU | | | CCACTCAAAAATAAAT | CCACUCAAAAAUAAAU |
| [SEQ ID | [SEQ ID | [SEQ ID | [SEQ ID | [SEQ ID | | | AACTTCCAGTTTTAGT | AACUUCCAGUUUUAGU |
| NO: 738] | NO: 1266] | NO: 854] | NO: 1382] | NO: 1086] | | | GAAATATATATTAAAA | GAAAUAUAUAUUAAAA |
| | | | | | | | AACTGGAAGCCATTTA | AACUGGAAGCCAUUUA |
| | | | | | | | TTTTATAGTGTGACAG | UUUUAUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 970] | [SEQ ID NO: 1498] |
| ATGACTTTCAAG | AUGACUUUCAAG | TTTAATAACCAC | UUUAAUAACCAC | UUUAAUAACCCU | -2.77085 | 4390 | ACCGGACATACTTGTT | ACCGGACAUACUUGUU |
| GGTTATTAAA | GGUUAUUAAA | TGAAAGTCAA | UGAAAGUCAA | UGAAAGUC | | | CCACTCATGACTTTCA | CCACUCAUGACUUUCA |
| [SEQ ID | [SEQ ID | [SEQ ID | [SEQ ID | [SEQ ID | | | AGGGTTATTAAATAGT | AGGGUUAUUAAAUAGU |
| NO: 739] | NO: 1267] | NO: 855] | NO: 1383] | NO: 1087] | | | GAAATATATATTAAAT | GAAAUAUAUAUUAAAU |
| | | | | | | | TTAATAACCACTGAAA | UUAAUAACCACUGAAA |
| | | | | | | | GTCAATAGTGTGACAG | GUCAAUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 971] | [SEQ ID NO: 1499] |
| TATGACTTTCAA | UAUGACUUUCAA | TTAATAACCCCC | UUAAUAACCCCC | UUAAUAACCCUU | -3.75832 | 4391 | ACCGGACATACTTGTT | ACCGGACAUACUUGUU |
| GGGTTATTAA | GGGUUAUUAA | GAAAGTCATT | GAAAGUCAUU | GAAAGUCA | | | CCACTCTATGACTTTC | CCACUCUAUGACUUUC |
| [SEQ ID | [SEQ ID | [SEQ ID | [SEQ ID | [SEQ ID | | | AAGGGTTATTAATAGT | AAGGGUUAUUAAUAGU |
| NO: 740] | NO: 1268] | NO: 856] | NO: 1384] | NO: 1088] | | | GAAATATATATTAAAT | GAAAUAUAUAUUAAAU |
| | | | | | | | TAATAACCCCCGAAAG | UAAUAACCCCCGAAAG |
| | | | | | | | TCATTTAGTGTGACAG | UCAUUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 972] | [SEQ ID NO: 1500] |
| TCATGACTTTCA | UCAUGACUUUCA | TAATAACCCTCT | UAAUAACCCUCU | UAAUAACCCUUG | -4.61593 | 4392 | ACCGGACATACTTGTT | ACCGGACAUACUUGUU |
| AGGGTTATTA | AGGGUUAUUA | AAAGTCATGT | AAAGUCAUGU | AAAGUCAU | | | CCACTCTCATGACTTT | CCACUCUCAUGACUUU |
| [SEQ ID | [SEQ ID | [SEQ ID | [SEQ ID | [SEQ ID | | | CAAGGGTTATTATAGT | CAAGGGUUAUUAUAGU |
| NO: 741] | NO: 1269] | NO: 857] | NO: 1385] | NO: 1089] | | | GAAATATATATTAAAT | GAAAUAUAUAUUAAAU |
| | (same as | | | | | | AATAACCCTCTAAAGT | AAUAACCCUCUAAAGU |
| | guide in XD- | | | | | | CATGTTAGTGTGACAG | CAUGUUAGUGUGACAG |
| | 14933) | | | | | | GGATACAGCAACTATT | GGAUACAGCAACUAUU |
| | | | | | | | TTATCAA | UUAUCAA |
| | | | | | | | [SEQ ID NO: 973] | [SEQ ID NO: 1501] |
| TTCATGACTTTC | UUCAUGACUUUC | AATAACCCTTTG | AAUAACCCUUUG | AAUAACCCUUGA | -3.88921 | 4393 | ACCGGACATACTTGTT | ACCGGACAUACUUGUU |
| AAGGGTTATT | AAGGGUUAUU | AAGTCATGAT | AAGUCAUGAU | AAGUCAUG | | | CCACTCTTCATGACTT | CCACUCUUCAUGACUU |
| [SEQ ID | [SEQ ID | [SEQ ID | [SEQ ID | [SEQ ID | | | TCAAGGGTTATTTAGT | UCAAGGGUUAUUUAGU |
| NO: 742] | NO: 1270] | NO: 858] | NO: 1386] | NO: 1090] | | | GAAATATATATTAAAA | GAAAUAUAUAUUAAAA |
| | | | | | | | ATAACCCTTTGAAGTC | AUAACCCUUUGAAGUC |
| | | | | | | | ATGATTAGTGTGACAG | AUGAUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 974] | [SEQ ID NO: 1502] |
| TTTCATGACTTT | UUUCAUGACUUU | ATAACCCTTGGG | AUAACCCUUGGG | AUAACCCUUGAA | -3.57278 | 4394 | ACCGGACATACTTGTT | ACCGGACAUACUUGUU |
| CAAGGGTTAT | CAAGGGUUAU | AGTCATGAAT | AGUCAUGAAU | AGUCAUGA | | | CCACTCTTTCATGACT | CCACUCUUUCAUGACU |
| [SEQ ID | [SEQ ID | [SEQ ID | [SEQ ID | [SEQ ID | | | TTCAAGGGTTATTAGT | UUCAAGGGUUAUUAGU |
| NO: 743] | NO: 1271] | NO: 859] | NO: 1387] | NO: 1091] | | | GAAATATATATTAAAA | GAAAUAUAUAUUAAAA |
| | | | | | | | TAACCCTTGGGAGTCA | UAACCCUUGGGAGUCA |
| | | | | | | | TGAATTAGTGTGACAG | UGAAUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 975] | [SEQ ID NO: 1503] |
| TGTTCATGACTT | UGUUCAUGACUU | TAACCCTTGAGG | UAACCCUUGAGG | UAACCCUUGAAA | -4.31654 | 4395 | ACCGGACATACTTGTT | ACCGGACAUACUUGUU |
| TCAAGGGTTA | UCAAGGGUUA | GTCATGAACT | GUCAUGAACU | GUCAUGAA | | | CCACTCTGTTCATGAC | CCACUCUGUUCAUGAC |
| [SEQ ID | [SEQ ID | [SEQ ID | [SEQ ID | [SEQ ID | | | TTTCAAGGGTTATAGT | UUUCAAGGGUUAUAGU |
| NO: 744] | NO: 1272] | NO: 860] | NO: 1388] | NO: 1092] | | | GAAATATATATTAAAT | GAAAUAUAUAUUAAAU |
| | | | | | | | AACCCTTGAGGGTCAT | AACCCUUGAGGGUCAU |
| | | | | | | | GAACTTAGTGTGACAG | GAACUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 976] | [SEQ ID NO: 1504] |
| TTGTTCATGACT | UUGUUCAUGACU | AACCCTTGAAGT | AACCCUUGAAGU | AACCCUUGAAAG | -3.76092 | 4396 | ACCGGACATACTTGTT | ACCGGACAUACUUGUU |
| TTCAAGGGTT | UUCAAGGGUU | TCATGAACAT | UCAUGAACAU | UCAUGAAC | | | CCACTCTTGTTCATGA | CCACUCUUGUUCAUGA |
| [SEQ ID | [SEQ ID | [SEQ ID | [SEQ ID | [SEQ ID | | | CTTTCAAGGGTTTAGT | CUUUCAAGGGUUUAGU |
| NO: 745] | NO: 1273] | NO: 861] | NO: 1389] | NO: 1093] | | | GAAATATATATTAAAA | GAAAUAUAUAUUAAAA |
| | | | | | | | ACCCTTGAAGTTCATG | ACCCUUGAAGUUCAUG |
| | | | | | | | AACATTAGTGTGACAG | AACAUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 977] | [SEQ ID NO: 1505] |
| TGTGTTCATGAC | UGUGUUCAUGAC | ACCCTTGAAATC | ACCCUUGAAAUC | ACCCUUGAAAGU | -4.07971 | 4397 | ACCGGACATACTTGTT | ACCGGACAUACUUGUU |
| TTTCAAGGGT | UUUCAAGGGU | CATGAACACT | CAUGAACACU | CAUGAACA | | | CCACTCTGTGTTCATG | CCACUCUGUGUUCAUG |
| [SEQ ID | [SEQ ID | [SEQ ID | [SEQ ID | [SEQ ID | | | ACTTTCAAGGGTTAGT | ACUUUCAAGGGUUAGU |
| NO: 746] | NO: 1274] | NO: 862] | NO: 1390] | NO: 1094] | | | GAAATATATATTAAAA | GAAAUAUAUAUUAAAA |
| | | | | | | | CCCTTGAAATCCATGA | CCCUUGAAAUCCAUGA |
| | | | | | | | ACACTTAGTGTGACAG | ACACUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 978] | [SEQ ID NO: 1506] |
| ATGTGTTCATGA | AUGUGUUCAUGA | CCCTTGAAAGCA | CCCUUGAAAGCA | CCCUUGAAAGUC | -4.76135 | 4398 | ACCGGACATACTTGTT | ACCGGACAUACUUGUU |
| CTTTCAAGGG | CUUUCAAGGG | ATGAACACAA | AUGAACACAA | AUGAACAC | | | CCACTCATGTGTTCAT | CCACUCAUGUGUUCAU |
| [SEQ ID | [SEQ ID | [SEQ ID | [SEQ ID | [SEQ ID | | | GACTTTCAAGGGTAGT | GACUUUCAAGGGUAGU |
| NO: 747] | NO: 1275] | NO: 863] | NO: 1391] | NO: 1095] | | | GAAATATATATTAAAC | GAAAUAUAUAUUAAAC |
| | | | | | | | CCTTGAAAGCAATGAA | CCUUGAAAGCAAUGAA |
| | | | | | | | CACAATAGTGTGACAG | CACAAUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 979] | [SEQ ID NO: 1507] |
| TATGTGTTCATG | UAUGUGUUCAUG | CCTTGAAAGTAG | CCUUGAAAGUAG | CCUUGAAAGUCA | -4.33797 | 4399 | ACCGGACATACTTGTT | ACCGGACAUACUUGUU |
| ACTTTCAAGG | ACUUUCAAGG | TGAACACATT | UGAACACAUU | UGAACACA | | | CCACTCTATGTGTTCA | CCACUCUAUGUGUUCA |
| [SEQ ID | [SEQ ID | [SEQ ID | [SEQ ID | [SEQ ID | | | TGACTTTCAAGGTAGT | UGACUUUCAAGGUAGU |
| NO: 748] | NO: 1276] | NO: 864] | NO: 1392] | NO: 1096] | | | GAAATATATATTAAAC | GAAAUAUAUAUUAAAC |
| | | | | | | | CTTGAAAGTAGTGAAC | CUUGAAAGUAGUGAAC |
| | | | | | | | ACATTTAGTGTGACAG | ACAUUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 980] | [SEQ ID NO: 1508] |
| TGATGTGTTCAT | UGAUGUGUUCAU | CTTGAAAGTCGC | CUUGAAAGUCGC | CUUGAAAGUCAU | -4.13084 | 4400 | ACCGGACATACTTGTT | ACCGGACAUACUUGUU |
| GACTTTCAAG | GACUUUCAAG | GAACACATCT | GAACACAUCU | GAACACAU | | | CCACTCTGATGTGTTC | CCACUCUGAUGUGUUC |
| [SEQ ID | [SEQ ID | [SEQ ID | [SEQ ID | [SEQ ID | | | ATGACTTTCAAGTAGT | AUGACUUUCAAGUAGU |
| NO: 749] | NO: 1277] | NO: 865] | NO: 1393] | NO: 1097] | | | GAAATATATATTAAAC | GAAAUAUAUAUUAAAC |
| | | | | | | | TTGAAAGTCGCGAACA | UUGAAAGUCGCGAACA |
| | | | | | | | CATCTTAGTGTGACAG | CAUCUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 981] | [SEQ ID NO: 1509] |
| TTGATGTGTTCA | UUGAUGUGUUCA | TTGAAAGTCACT | UUGAAAGUCACU | UUGAAAGUCAUG | -4.24964 | 4401 | ACCGGACATACTTGTT | ACCGGACAUACUUGUU |
| TGACTTTCAA | UGACUUUCAA | AACACATCAT | AACACAUCAU | AACACAUC | | | CCACTCTTGATGTGTT | CCACUCUUGAUGUGUU |
| [SEQ ID | [SEQ ID | [SEQ ID | [SEQ ID | [SEQ ID | | | CATGACTTTCAATAGT | CAUGACUUUCAAUAGU |
| NO: 750] | NO: 1278] | NO: 866] | NO: 1394] | NO: 1098] | | | GAAATATATATTAAAT | GAAAUAUAUAUUAAAU |
| | | | | | | | TGAAAGTCACTAACAC | UGAAAGUCACUAACAC |
| | | | | | | | ATCATTAGTGTGACAG | AUCAUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 982] | [SEQ ID NO: 1510] |
| TCTGATGTGTTC | UCUGAUGUGUUC | TGAAAGTCATTG | UGAAAGUCAUUG | UGAAAGUCAUGA | -4.95673 | 4402 | ACCGGACATACTTGTT | ACCGGACAUACUUGUU |
| ATGACTTTCA | AUGACUUUCA | ACACATCAGT | ACACAUCAGU | ACACAUCA | | | CCACTCTCTGATGTGT | CCACUCUCUGAUGUGU |
| [SEQ ID | [SEQ ID | [SEQ ID | [SEQ ID | [SEQ ID | | | TCATGACTTTCATAGT | UCAUGACUUUCAUAGU |
| NO: 751] | NO: 1279] | NO: 867] | NO: 1395] | NO: 1099] | | | GAAATATATATTAAAT | GAAAUAUAUAUUAAAU |
| | | | | | | | GAAAGTCATTGACACA | GAAAGUCAUUGACACA |
| | | | | | | | TCAGTTAGTGTGACAG | UCAGUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 983] | [SEQ ID NO: 1511] |
| AGCTGATGTGTT | AGCUGAUGUGUU | GAAAGTCATGGG | GAAAGUCAUGGG | GAAAGUCAUGAA | -3.87865 | 4403 | ACCGGACATACTTGTT | ACCGGACAUACUUGUU |
| CATGACTTTC | CAUGACUUUC | CACATCAGCA | CACAUCAGCA | CACAUCAG | | | CCACTCAGCTGATGTG | CCACUCAGCUGAUGUG |
| [SEQ ID | [SEQ ID | [SEQ ID | [SEQ ID | [SEQ ID | | | TTCATGACTTTCTAGT | UUCAUGACUUUCUAGU |
| NO: 752] | NO: 1280] | NO: 868] | NO: 1396] | NO: 1100] | | | GAAATATATATTAAAG | GAAAUAUAUAUUAAAG |
| | | | | | | | AAAGTCATGGGCACAT | AAAGUCAUGGGCACAU |
| | | | | | | | CAGCATAGTGTGACAG | CAGCAUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 984] | [SEQ ID NO: 1512] |
| TAGCTGATGTGT | UAGCUGAUGUGU | AAAGTCATGAGA | AAAGUCAUGAGA | AAAGUCAUGAAC | -3.03908 | 4404 | ACCGGACATACTTGTT | ACCGGACAUACUUGUU |
| TCATGACTTT | UCAUGACUUU | ACATCAGCTT | ACAUCAGCUU | ACAUCAGC | | | CCACTCTAGCTGATGT | CCACUCUAGCUGAUGU |
| [SEQ ID | [SEQ ID | [SEQ ID | [SEQ ID | [SEQ ID | | | GTTCATGACTTTTAGT | GUUCAUGACUUUUAGU |
| NO: 753] | NO: 1281] | NO: 869] | NO: 1397] | NO: 1101] | | | GAAATATATATTAAAA | GAAAUAUAUAUUAAAA |
| | | | | | | | AAGTCATGAGAACATC | AAGUCAUGAGAACAUC |
| | | | | | | | AGCTTTAGTGTGACAG | AGCUUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 985] | [SEQ ID NO: 1513] |
| TTAGCTGATGTG | UUAGCUGAUGUG | AAGTCATGAAAG | AAGUCAUGAAAG | AAGUCAUGAACA | -4. 75357 | 4405 | ACCGGACATACTTGTT | ACCGGACAUACUUGUU |
| TTCATGACTT | UUCAUGACUU | CATCAGCTAT | CAUCAGCUAU | CAUCAGCU | | | CCACTCTTAGCTGATG | CCACUCUUAGCUGAUG |
| [SEQ ID | [SEQ ID | [SEQ ID | [SEQ ID | [SEQ ID | | | TGTTCATGACTTTAGT | UGUUCAUGACUUUAGU |
| NO: 754] | NO: 1282] | NO: 870] | NO: 1398] | NO: 1102] | | | GAAATATATATTAAAA | GAAAUAUAUAUUAAAA |
| | | | | | | | AGTCATGAAAGCATCA | AGUCAUGAAAGCAUCA |
| | | | | | | | GCTATTAGTGTGACAG | GCUAUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 986] | [SEQ ID NO: 1514] |
| TCTAGCTGATGT | UCUAGCUGAUGU | AGTCATGAACGA | AGUCAUGAACGA | AGUCAUGAACAC | -5.05901 | 4406 | ACCGGACATACTTGTT | ACCGGACAUACUUGUU |
| GTTCATGACT | GUUCAUGACU | ATCAGCTAGT | AUCAGCUAGU | AUCAGCUA | | | CCACTCTCTAGCTGAT | CCACUCUCUAGCUGAU |
| [SEQ ID | [SEQ ID | [SEQ ID | [SEQ ID | [SEQ ID | | | GTGTTCATGACTTAGT | GUGUUCAUGACUUAGU |
| NO: 755] | NO: 1283] | NO: 871] | NO: 1399] | NO: 1103] | | | GAAATATATATTAAAA | GAAAUAUAUAUUAAAA |
| | | | | | | | GTCATGAACGAATCAG | GUCAUGAACGAAUCAG |
| | | | | | | | CTAGTTAGTGTGACAG | CUAGUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 987] | [SEQ ID NO: 1515] |
| TGCTAGCTGATG | UGCUAGCUGAUG | GTCATGAACAAG | GUCAUGAACAAG | GUCAUGAACACA | -4.47567 | 4407 | ACCGGACATACTTGTT | ACCGGACAUACUUGUU |
| TGTTCATGAC | UGUUCAUGAC | TCAGCTAGCT | UCAGCUAGCU | UCAGCUAG | | | CCACTCTGCTAGCTGA | CCACUCUGCUAGCUGA |
| [SEQ ID | [SEQ ID | [SEQ ID | [SEQ ID | [SEQ ID | | | TGTGTTCATGACTAGT | UGUGUUCAUGACUAGU |
| NO: 756] | NO: 1284] | NO: 872] | NO: 1400] | NO: 1104] | | | GAAATATATATTAAAG | GAAAUAUAUAUUAAAG |
| | | | | | | | TCATGAACAAGTCAGC | UCAUGAACAAGUCAGC |
| | | | | | | | TAGCTTAGTGTGACAG | UAGCUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 988] | [SEQ ID NO: 1516] |
| TTGCTAGCTGAT | UUGCUAGCUGAU | TCATGAACACGC | UCAUGAACACGC | UCAUGAACACAU | -4.42302 | 4408 | ACCGGACATACTTGTT | ACCGGACAUACUUGUU |
| GTGTTCATGA | GUGUUCAUGA | CAGCTAGCAT | CAGCUAGCAU | CAGCUAGC | | | CCACTCTTGCTAGCTG | CCACUCUUGCUAGCUG |
| [SEQ ID | [SEQ ID | [SEQ ID | [SEQ ID | [SEQ ID | | | ATGTGTTCATGATAGT | AUGUGUUCAUGAUAGU |
| NO: 757] | NO: 1285] | NO: 873] | NO: 1401] | NO: 1105] | | | GAAATATATATTAAAT | GAAAUAUAUAUUAAAU |
| | | | | | | | CATGAACACGCCAGCT | CAUGAACACGCCAGCU |
| | | | | | | | AGCATTAGTGTGACAG | AGCAUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 989] | [SEQ ID NO: 1517] |
| TTTGCTAGCTGA | UUUGCUAGCUGA | CATGAACACACA | CAUGAACACACA | CAUGAACACAUC | -5.35102 | 4409 | ACCGGACATACTTGTT | ACCGGACAUACUUGUU |
| TGTGTTCATG | UGUGUUCAUG | AGCTAGCAAT | AGCUAGCAAU | AGCUAGCA | | | CCACTCTTTGCTAGCT | CCACUCUUUGCUAGCU |
| [SEQ ID | [SEQ ID | [SEQ ID | [SEQ ID | [SEQ ID | | | GATGTGTTCATGTAGT | GAUGUGUUCAUGUAGU |
| NO: 758] | NO: 1286] | NO: 874 ] | NO: 1402] | NO: 1106] | | | GAAATATATATTAAAC | GAAAUAUAUAUUAAAC |
| | (same as | | | | | | ATGAACACACAAGCTA | AUGAACACACAAGCUA |
| | guide in XD- | | | | | | GCAATTAGTGTGACAG | GCAAUUAGUGUGACAG |
| | 14934) | | | | | | GGATACAGCAACTATT | GGAUACAGCAACUAUU |
| | | | | | | | TTATCAA | UUAUCAA |
| | | | | | | | [SEQ ID NO: 990] | [SEQ ID NO: 1518] |
| TTTTGCTAGCTG | UUUUGCUAGCUG | ATGAACACATAG | AUGAACACAUAG | AUGAACACAUCA | -0.73644 | 4410 | ACCGGACATACTTGTT | ACCGGACAUACUUGUU |
| ATGTGTTCAT | AUGUGUUCAU | GCTAGCAAAT | GCUAGCAAAU | GCUAGCAA | | | CCACTCTTTTGCTAGC | CCACUCUUUUGCUAGC |
| [SEQ ID | [SEQ ID | [SEQ ID | [SEQ ID | [SEQ ID | | | TGATGTGTTCATTAGT | UGAUGUGUUCAUUAGU |
| NO: 759] | NO: 1287] | NO: 875] | NO: 1403] | NO: 1107] | | | GAAATATATATTAAAA | GAAAUAUAUAUUAAAA |
| | | | | | | | TGAACACATAGGCTAG | UGAACACAUAGGCUAG |
| | | | | | | | CAAATTAGTGTGACAG | CAAAUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 991] | [SEQ ID NO: 1519] |
| TCTTTTGCTAGC | UCUUUUGCUAGC | GAACACATCATA | GAACACAUCAUA | GAACACAUCAGC | -1.29476 | 4412 | ACCGGACATACTTGTT | ACCGGACAUACUUGUU |
| TGATGTGTTC | UGAUGUGUUC | TAGCAAAAGT | UAGCAAAAGU | UAGCAAAA | | | CCACTCTCTTTTGCTA | CCACUCUCUUUUGCUA |
| [SEQ ID | [SEQ ID | [SEQ ID | [SEQ ID | [SEQ ID | | | GCTGATGTGTTCTAGT | GCUGAUGUGUUCUAGU |
| NO: 760] | NO: 1288] | NO: 876] | NO: 1404] | NO: 1108] | | | GAAATATATATTAAAG | GAAAUAUAUAUUAAAG |
| | | | | | | | AACACATCATATAGCA | AACACAUCAUAUAGCA |
| | | | | | | | AAAGTTAGTGTGACAG | AAAGUUAGUGUGACAG |
| | | | | | | | GGATACAGCAACTATT | GGAUACAGCAACUAUU |
| | | | | | | | TTATCAA | UUAUCAA |
| | | | | | | | [SEQ ID NO: 992] | [SEQ ID NO: 1520] |
| TGTCAAGTTTAG | | CGCCCTTTTAAC | | CGCCCUUUUACU | -4.72 | 4502 | ACCGGACATACTTGTT | |
| TAAAAGGGCG | | AAACTTGACT | | AAACUUGA | | | CCACTCTGTCAAGTTT | |
| [SEQ ID | | [SEQ ID | | [SEQ ID | | | AGTAAAAGGGCGTAGT | |
| NO: 2269] | | NO: 2270] | | NO: 2271] | | | GAAATATATATTAAAC | |
| | | | | | | | GCCCTTTTAACAAACT | |
| | | | | | | | TGACTTAGTGTGACAG | |
| | | | | | | | GGATACAGCAACTATT | |
| | | | | | | | TTATCAA | |
| | | | | | | | [SEQ ID NO: 2272] | |

### Example 3: Testing of Top Hits from Pooled Screen in Lentiviral Transduction of Human Neurons

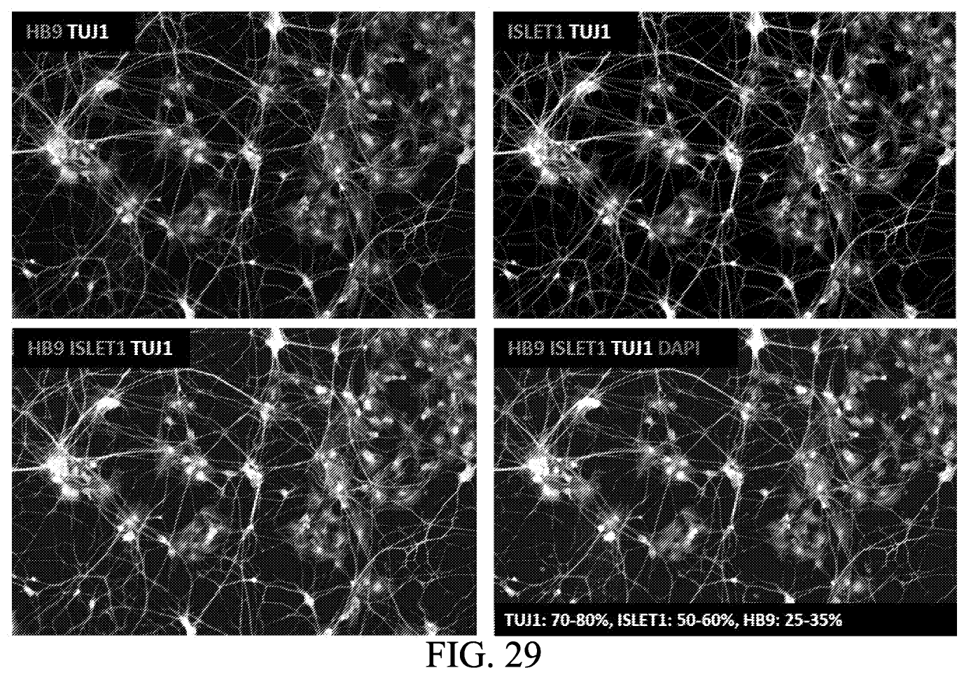

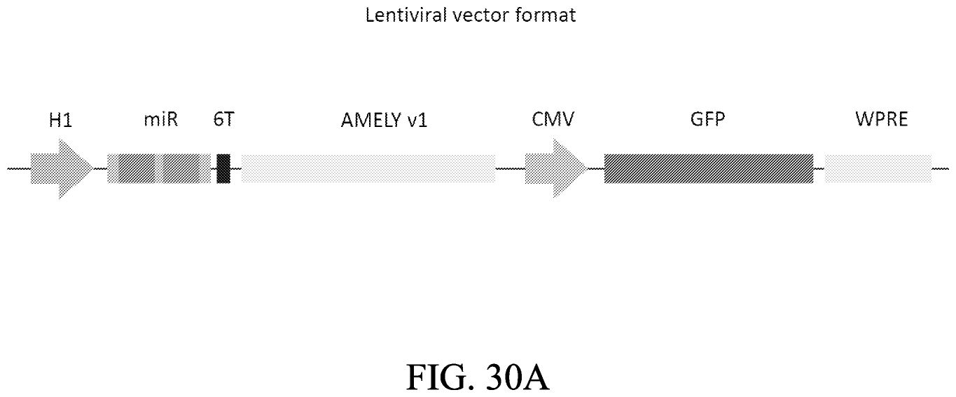

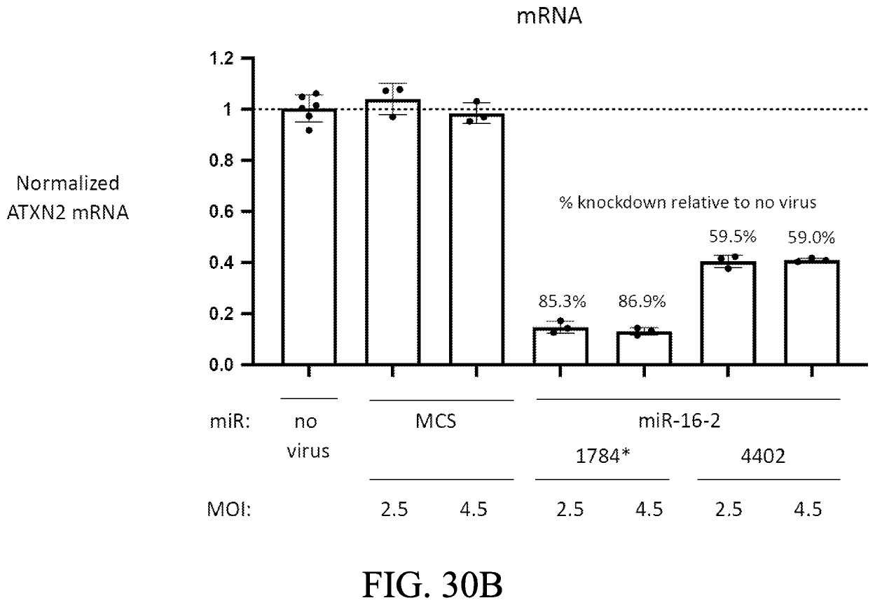

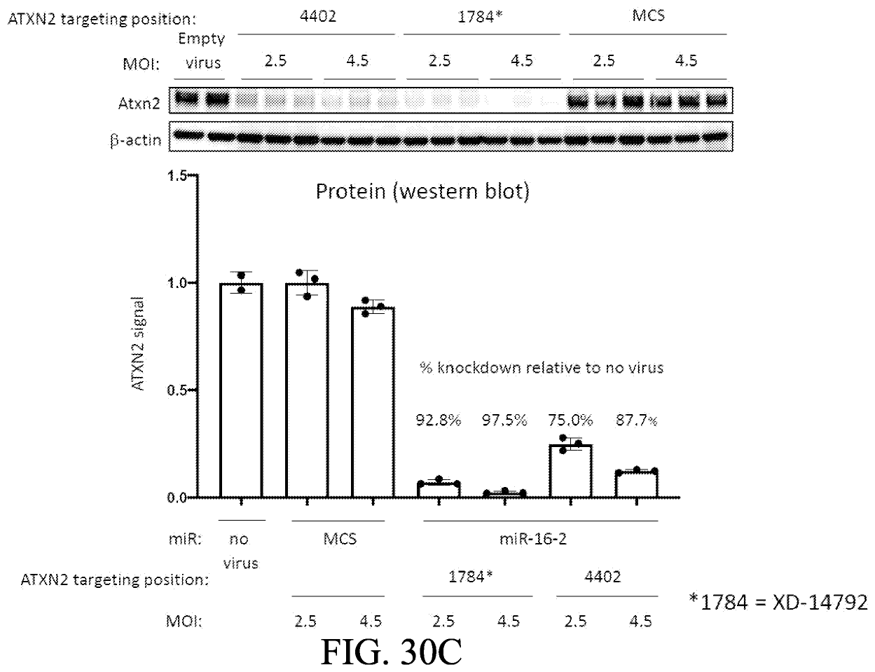

Several top hits from pooled Deep Screen 1 (Example 2) were cloned into lentiviral vectors, packaged, and tested in stem-cell derived motor neuron cultures for knockdown of ATXN2 mRNA and protein. An example lentiviral vector is given in H1-miR-16-2_1755-AMELY_V1_CMV_GFP_lenti (SEQ ID NO:1521) which contains a amiRNA targeting position 1755 of ATXN2 transcript embedded in a miR-16-2 backbone, or the other vectors described here. The amiRNA sequence in the vector (e.g., nucleotides 1889-2020 of SEQ ID NO:1521) may replaced with the corresponding amiR or control non-miRNA sequence (MCS) but the rest of the vector is left unchanged.) Characterization of motor neurons (FIG. 29) shows that cultures (differentiation protocol described in below methods) generated cultures enriched for motor neurons, with elaborated neuronal processes. amiRNAs were embedded in lentiviral vectors (FIG. 30A) with an H1 promoter as well as a GFP expression cassette. In a first experiment, two amiRNAs, targeting ATXN2 at position 1784 (guide sequence SEQ ID NO:112) in the coding sequence or ATXN2 at position 4402 (guide sequence SEQ ID NO:1279) having miR-16-2 backbones were tested at two different doses. Strong knockdown of ATXN2 mRNA and protein was detected by qPCR analysis of mRNA and Western analysis of protein, respectively (FIGS. 30B-30C). Protein levels as measured in this assay showed a greater fractional reduction of protein levels than mRNA levels, indicating that measurements of mRNA may represent at least the amount of ATXN2 protein reduced by a given amiRNA. Surprisingly, the amiRNA targeting the ATXN2 coding sequence (1784) yielded greater knockdown than the amiRNA targeting the 3′ UTR (4402), which is different than the relative performance of those amiRNAs in Deep Screen 1.

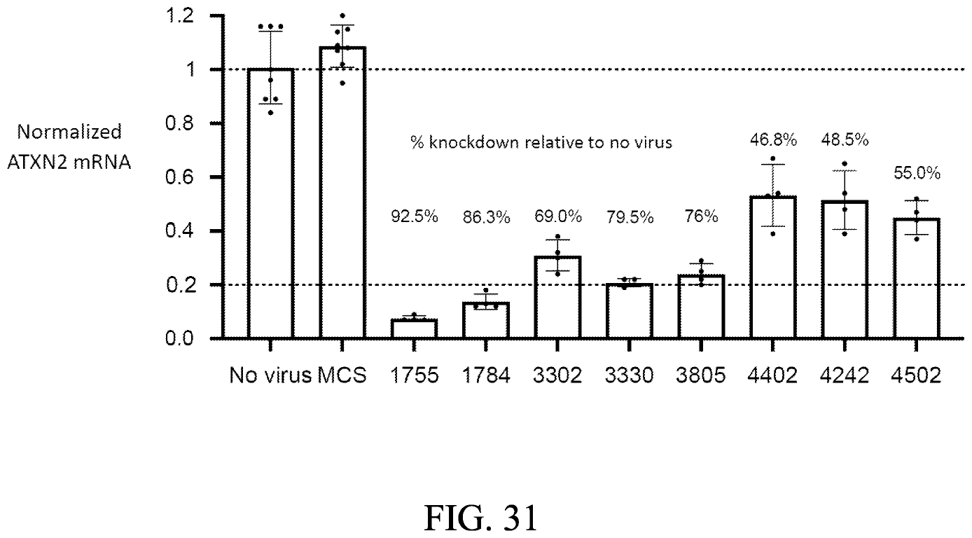

As a further investigation of amiRNA targeting the coding region versus the 3′ UTR, a second experiment was done (FIG. 31). In this case, all neurons were treated at a dose intermediate between the two levels tested in the first human neuron lentiviral dosing experiment. As before, amiRNAs targeting the coding sequence (1755 (guide sequence SEQ ID NO:1185), 1784 (guide sequence SEQ ID NO:112), 3302 (guide sequence SEQ ID NO:1216), 3330 (guide sequence SEQ ID NO:1811), and 3805 (guide sequence SEQ ID NO:1221) yielded stronger knockdown than amiRNAs targeting the 3′ UTR (4402 (guide sequence SEQ ID NO:1279), 4242 (guide sequence SEQ ID NO:1233), and 4502) in these neuronal cultures. The amount of mRNA reduction exceeded 75% for some amiRNAs, such as 1755 (guide sequence SEQ ID NO:1185), 1784 (guide sequence SEQ ID NO:112) and 3330 (guide sequence SEQ ID NO:1811).

### Methods

### Motor Neuron Production

Induced pluripotent stem cells (GM25256, Coriell Institute) were cultured in feeder-free conditions, in mTeSR1 media on Matrigel coated plates, according to standard procedures. To begin differentiation, iPSC colonies grown in 6-well dishes were dissociated with 500 uL ReLeSR, incubating 3 minutes at 37 C, and gently agitated. 1 mL of complete mTeSR1 media is added to stop dissociation. Cell suspension was collected, ReLeSR removed and cells resuspended in N2B27 differentiation media: 50 mL of 50% mTeSR1 and 50% NB27 differentiation media (50% DMEM-F12, 50% Neurobasal medium, 1×N-2 supplement, 1×B-27 supplement, XenoFree, 0.5× penicillin-streptomycin, 1×2-mercaptoethanol, 20 uM L-ascorbic acid). Rock Inhibitor Y-27632 (5 micromolar), LDN (200 nM), SB 431542 (40 micromolar), and Chir 99021 (3 micromolar) were added. Cell suspension was then transferred to a 75 cm² ultra low attachment U-flask for 24 hours. Cells then aggregated into small spheroids.

Media changes were then performed on days 2, 4, 6, 9, and 12. Media included (all based in N2B27 differentiation media): Day 2: Retinoic acid (1 micromolar), SAG (1 micromolar), LDN-193189 (0.2 micromolar), SB 431542 hydrate (40 micromolar), CHIR 99021 (3 micromolar). Day 4: Retinoic acid (1 micromolar), SAG (1 micromolar), LDN-193189 (0.2 micromolar). Day 6: Retinoic acid (1 micromolar); SAG (1 micromolar). Day 9: Retinoic acid (1 micromolar), SAG (1 micromolar), DAPT (10 micromolar). Day 12: DAPT (10 micromolar). By day 14, neuronal spheroids were present and were dissociated to plate motor neurons.

Neuronal spheroids were then dissociated with a papain:DNAse solution and triturated 4-5×. Cell suspensions were then divided into wells of 6-well plates; and after a 15 minute incubation, further triturated. Following this dissociation, enzyme was inactivated with a DMEM and knockout serum replacement (KOSR) mix, centrifuged, washed again in 90% DMEM/10% KOSR, centrifuged, and resuspended in complete neurobasal media: Neurobasal medium, 1×N-2 supplement, 1×B-27 supplement, XenoFree, 0.5× penicillin-streptomycin, 20 uM L-ascorbic acid, 1% KOSR, Rock Inhibitor Y-27632 (5 micromolar), GDNF (10 ng/mL), BDNF (20 ng/mL), CNTF (10 ng/mL), DAPT (5 micromolar). Cells were then centrifuged again, resuspended in complete neurobasal media, passed through a 40 micron cell strainer, counted via trypan blue staining and a hemocytometer, then diluted to 20K/well (96-well format) or 200K/well (24-well format) for plating in PDL/Laminin coated plates. Cells were cultured in a volume of neurobasal media: 200 uL/well (96-well format) or 1 mL/well (24-well format).

The PDL/Laminin coating was done by treating plates with a 100 microgram/mL solution of poly-D-lysine in PBS overnight at 4 C; washing 3 times with PBS; then treating plates overnight at 4 C with a 50 microgram/mL solution of laminin in PBS.

48 hours after plating, 50% of media was replaced with neuron maintenance media (Neurobasal, with 1×N-2 supplement, 1×Xeno-Free B-27 supplement, 0.5× penicillin-streptomycin, 20 micromolar L-ascorbic acid, with 10 ng/mL GDNF, 10 ng/mL BDNF, 10 ng/mL CNTF), including DAPT (5 micromolar). Thereafter, 50% of media was replaced 3 times per week, not including DAPT.

References relevant to the above protocol include: (Highly efficient neural conversion of human ES and iPS cells by dual inhibition of SMAD signaling (Chambers et al., Nat Biotechnol (2009) 27:275-280) and (Maury et al., Nat Biotechnol (2014) 33:89-96).

Reagents and Equipment for iPSC Embryoid Body Formation

| Reagent | Vendor | Cat. No. |
|---|---|---|
| DMEM/F-12 | ThermoFisher | 11320-033 |
| Neurobasal Medium | | 21103049 |
| N-2 Supplement (100X) | | 17502-048 |
| B-27 Supplement (50X), XenoFree | | A1486701 |
| Phosphate-Buffered Saline (PBS) pH 7.4 | | 10010023 |
| Penicillin-Streptomycin 100x | | 15140122 |
| 2-Mercaptoethanol 1000x | | 21985023 |
| L-Ascorbic Acid | Millipore Sigma | A4403- |
| | | 100MG |
| Rock Inhibitor (Y-27632), 5MG | | Y0503-5MG |
| SB 431542 hydrate | | S4317-5MG |
| Retinoic Acid | | R2625-50MG |
| DAPT | | D5942-5MG |
| SAG, SHH agonist | | 566660-5MG |
| MTeSR1 cGMP, feeder-free maintenance | StemCell | 85850 |
| medium kit | Technologies, | |
| ReLeSR | Inc | 05872 |
| LDN-193189 in solution | Fisher Scientific | NC0689818 |
| CHIR 99021 10MG | Tocris | 4423 |

| Equipment/Supply | Vendor | Cat. No. |
|---|---|---|
| Ultra-Low Attachment 75 cm² U-Flask | Corning | 3814 |
| 6-well plate | Corning | 353046 |
| Nalgene Rapid-Flow 500 mL Filter Units | VWR (Nalgene) | 73520-984 |

### Reagents and Embryoid Body Dissociation and Motor Neuron Culture:

| Reagent | Vendor | Cat. No. |
|---|---|---|
| Neurobasal Medium | ThermoFisher | 21103049 |
| DMEM/F-12 | | 11320-033 |
| N-2 Supplement (100X) | | 17502-048 |
| B-27 Supplement (50X), XenoFree | | A1486701 |
| 2-Mercaptoethanol 1000x | | 21985023 |
| Phosphate-Buffered Saline (PBS) pH 7.4 | | 10010-049 |
| Penicillin-Streptomycin 100x | | 15140122 |
| KnockOut Serum Replacement | | 10828010 |
| Laminin Mouse Protein, 1 mg in | | 23017015 |
| solution | | |
| Rock Inhibitor (Y-27632), 5 mg | Millipore Sigma | Y0503-5MG |
| DAPT | | D5942-5MG |
| L-Ascorbic Acid | | A4403- |
| | | 100MG |
| Poly-D-Lysine solution, 1 mg/mL | | A-003-E |
| Recombinant Human GDNF, 50 μg | R&D Systems | 212-GD-050 |
| Recombinant Human BDNF, 50 μg | | 248-BDB-050 |
| Recombinant Human CNTF, 50 μg | | 257-NT-050 |
| Papain, ≥100 units per vial | Worthington | LK003178 |
| DNase, ≥1,000 Kunitz units per vial | | LK003172 |

### Equipment for Embryoid Body Dissociation and Motor Neuron Culture

| Equipment/Supply | Vendor | Cat. No. |
|---|---|---|
| 6-well Clear TC-treated Plates | Corning | 353046 |
| 12-well Clear TC-treated Plates | Corning | 353043 |
| 24-well Clear TC-treated Plates | Corning | 353047 |
| 96-well optic clear bottom, black wall | PerkinElmer | 6005550 |
| microplate | | |
| Nalgene Rapid-Flow 500 mL Filter Units | VWR (Nalgene) | 73520-984 |

### Lentiviral Production

To test the efficacy of miR16-2 embedded guides in stem-cell derived motor neurons, amiRNAs were expressed from an H1 promoter embedded within a lentiviral construct as described above. Lentivirus was generated with Lenti-X 293T (Takara, 632180) cells transfected with psPAX2 (Cellecta, P/N CPCP-PAX2) and pMD2.2 (Cellecta, CPCP-PM2G) using Lipofectamine LTX and PLUS Reagent (Thermo, P/N 15338-100). The following day after transfection media was changed to include ViralBoost Reagent (Alstem, P/N VB100) and then 2 days later the viral production media was filtered and concentrated using Lenti-X Concentrator (Takara, P/N 631232) and resuspended in N2B27 media.

qPCR Analysis

Stem-cell derived motor neurons were transduced, and 7 days post-transduction, media was removed, washed with PBS and cells lysed with Buffer RLT supplemented with beta-Mercaptoethanol. RNA was purified using Qiagen RNeasy Plus Mini Kit (Qiagen, P/N 74134) and reverse-transcribed using SuperScript VILO cDNA Synthesis Kit (Thermo, P/N 11754250). Using TaqMan Fast Advanced Master Mix (Thermo, P/N 4444556) and QuantStudio 6 Flex Real-Time PCR System (Thermo), Ct values were calculated using primer/probe sets to ATXN2 (Thermo, Hs01002847_m1), GUSB (Thermo, Hs00939627_m1), and B2M (Thermo, Hs00187842_m1). The average Ct across 4 replicates was calculated, and using the delta-delta Ct method, the delta Ct was calculated for ATXN2 to each internal control, then the delta-delta Ct was calculated to the average of the untreated conditions. The mean of the normalized values to untreated conditions were calculated and graphed as shown.

Western Analysis of ATXN2 Levels from Neurons Treated with ATXN2 amiRNA Expressing Lentiviruses

Protein extraction was performed by placing plates on ice, aspirating media, and adding 50-100 microliters cold RIPA buffer (TEKNOVA #50-843-016) supplemented with protease and phosphatase inhibitor tablet (Pierce #A32959), Halt protease inhibitor cocktail (Thermo #1861279) and PMSF (Cell Signaling Technology #8553S). Individual cell lifters were used to scrape each well thoroughly, plates were tilted and lysates were harvested and incubated on ice for an additional 30 min. Samples were centrifuged for 15 min at 17,000×g at 4° C., and supernatant was transferred to a fresh tube and stored at −80° C. Protein lysates were quantitated (Pierce, 23225), resulting in approximately 40 μg total protein per sample.

The NuPage system (Thermo) was used for gel electrophoresis. Five μg of each sample was loaded onto 4-12% Bis-Tris protein gels (Thermo, NP0321BOX) and run at constant 200V for 1 hr. Revert 700 (Licor, 926-11010) was used to assay for protein loading. Proteins were transferred onto PVDF membrane (EMD Millipore, IPFL00005) overnight at 4° C. using constant 30V and 90 mA. Membranes were blocked for 1 hr at RT (Rockland, MB-070). Primary antibody incubation was performed overnight rocking at 4° C., including anti-Atxn2 (1:1000, BD, 611378), anti-GFP (1:2000, CST, 2956) and beta-actin (1:2000, CST, 4970). Washing was performed 4×5 min with TBS+0.1% tween-20, and secondary antibodies were incubated for 1 hr rocking at RT (1:15,000 each of 800CW goat anti-mouse and 680RD donkey anti-rabbit, Licor). Membranes were washed again and imaging was performed on an Odyssey Fc Imaging system (Licor). Signal quantitation was by Licor image-studio lite.

### Example 4: Embedding of Top Hits from Pooled Screen in AAV Cis-Plasmids and AAV Production

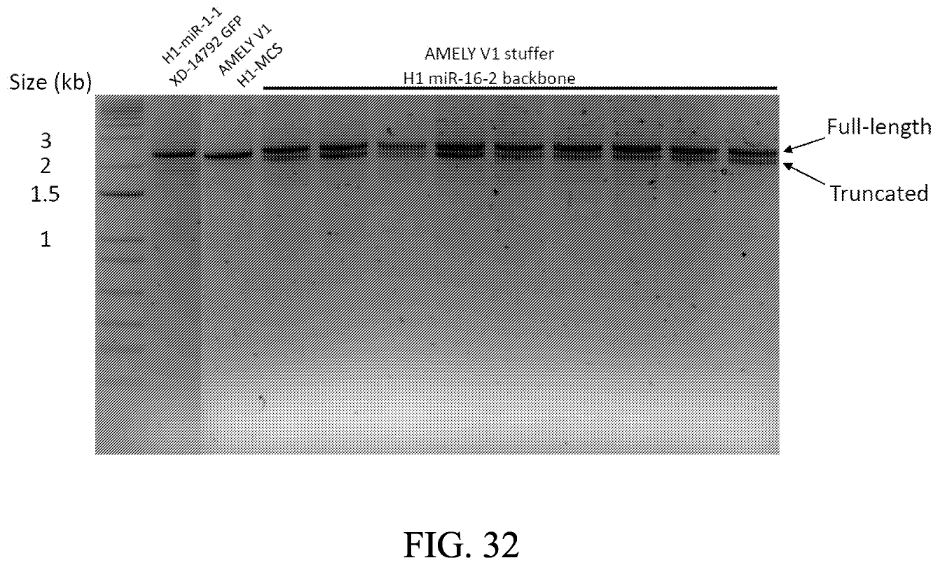

To test the ability of top performing amiRNAs identified from the pooled screen to knock down ATXN2 when embedded in AAV, 10 top miRNAs were cloned downstream of a H1 promoter (nucleotides 113-203 of SEQ ID NO:1522) in a cis plasmid (transfer plasmid) for AAV production. An example of a plasmid sequence (5′ ITR to 3′ ITR) (scAAV_AMELY_V1_H1_micropool_ITR_to_ITR) comprises the nucleotide sequence of SEQ ID NO:1522; where the desired amiRNA embedded in a miRNA backbone is inserted in nucleotides 204-341 of SEQ ID NO:1522. After AAV9 production by triple transfection of HEK293T cells with the cis-plasmid and helper plasmids and harvest of encapsidated AAV, vector genome DNA was extracted with Quick-DNA Viral Kit (Zymo, P/N D3015) to assess vector integrity. Purified vector was quantified using Qubit dsDNA HS Assay Kit (Thermo, P/N Q32854) and vector genome size was assessed by agarose gel electrophoresis and stained SyberSafe for visualization. Vector genome size was assessed by agarose gel electrophoresis (FIG. 32). Surprisingly, two bands were observed. The upper band migrated at the expected size 2284 bp, whereas the lower band migrated farther than the calculated vector size, or smaller in length than the full-length vector. Extraction of the band representing the full-length vector and subsequent Sanger sequencing with a primer amplifying towards the embedded aimRNA resulted in successful sequencing of the expected amiRNA. Whereas extraction of the smaller DNA product and sequencing failed to sequence through the embedded aimRNA, suggesting that the lower band might represent a vector truncation centered around the artificial miRNA, as noted in (Xie et al., Molecular Therapy (2013) 28:422-430). Calculation of predicted DNA secondary structure for miRs in the miR16-2 backbone using mfold (Zuker Nucleic Acids Research (2003) 31:3406-15) showed this sequence to form strong secondary structure, with Gibbs free binding energy of −26.78.

Using ImageJ, the individual vector genome lanes of an image gathered with the SyberSafe stained DNA gel were selected, the intensity of the lane plotted, and peaks quantified. Using the calculated lengths of the full-length and miR-centered truncated vector genomes of 2284 and 2077 bp respectively, the relative staining-intensity-derived molarity of each was calculated. With these values, the percentage full-length vector was calculated as the percentage of full-length divided by the combined amount of full-length and miR-centered truncated vector genomes (Table 20)

TABLE 20
Percentage of full-length vector genome

| H1 AMELY | percentage full-length |
|---|---|
| V1 guide | vector genome |
| miR16-2-XD-14792 | 68.3 |
| miR16-2-1479 | 56.7 |
| miR16-2-1755 | 70.3 |
| miR16-2-3330 | 62.5 |
| miR16-2-4402 | 64.1 |
| miR16-2-4405 | 67.8 |
| miR16-2-4406 | 71.0 |
| miR16-2-4409 | 67.2 |
| miR16-2-4502 | 64.9 |

### Example 5: Second Pooled miRNA Screen

Given the truncation observed in AAV vectors expressing miR16-2 embedded amiRNAs, a second pooled amiRNA screen was devised to embed the guide sequences from the top ATXN2 miRNA hits from the first pooled screen into a diverse set of 20 miRNA backbones.

### ATXN2 Targeting Sequence Selection for DS2

ATXN2 targeting sequences presumed to be efficacious and safe were selected from Deep Screen 1 to enter “Deep Screen 2.” Sequences that were enriched in the low ATXN2 signal FACS bin and demonstrated low dropout (minimal change in representation comparing an early to a late timepoint) were prioritized. To calibrate the dynamic range of the assay, some sequences with high dropout were additionally included. Since there may be biological variability in the processing precision of the mature guide strand, guides bracketing efficacious guides (by position along the ATXN2 transcript) were additionally entered into Deep Screen 2.

Essential Gene Control miRNA Selection

A subset of the essential gene targeting amiRNAs with either ‘high’ or ‘medium’ dropout, with respect to other essential-gene targeting amiRNAs, were selected for Deep Screen 2 based on performance in Deep Screen 1.

### 911 Controls

A subset of sequences targeting ATXN2 were paired with their cognate 911 controls. In a 911 control, bases 9, 10, and 11 of the guide strand are complemented, along with corresponding change in the passenger strand, such that the resulting mature miRNA does not slice the target mRNA of the original guide. Because many aspects of amiRNA ‘off-target’ activity are presumed to occur through binding interactions with the seed region (bases 2-8), these 911 controls should in principle display a similar off-target profile as the original miRNA and should help distinguish on- and off-target activity.

### ATXN2 Scramble Controls

A subset of the miRNA scramble controls from Deep Screen 1 was carried over into Deep Screen 2. These were considered for mean centering the data.

### ATXN2 Backbone Selection, Processing Enhancement Motifs, and Passenger Variations

MicroRNA backbones were selected for naturally exhibiting high processing precision, high guide to passenger ratio, and efficient target knockdown as an artificial miRNA. Both miRNA performance in functional screens and 5′ guide processing homogeneity were considered¹⁻⁴.

Primary miRNA transcript sequence was identified in miRbase. The extended sequence contexts around the miRNAs were ascertained in EntrezGene. Surrounding 5′ and 3′ sequence with high mammalian conservation were used to define final 138 nt miRNA-embedded fragments that would be inserted into the pooled library.

Mfold and RNAfold were used to examine folding patterns and to consider Gibbs free energy, as there is evidence that high Gibbs free energy derived from extensive secondary structure in the miRNA may produce miR-centered truncations when later cloned and produced into AAV.

The basal stem, loop, and guide and passenger sequences were defined by stem loop folding predictions on miRbase and Mfold. The rules for passenger variations such as bulges and other asymmetries were chosen to mimic non-complementary base pairing in the endogenous hairpin stem and incorporated into the library construction algorithms.

Sequence motifs that enable efficient processing of pri-miRNA backbones have previously been identified. These include an UG motif at the 5′ end of the pre-miRNA, a mismatched GHG motif in the stem, and a 3′ CNNC motif Many of the primary miRNA transcripts selected naturally contain these motifs. Some of these motifs were artificially incorporated into five backbones, and these resulting miRNA backbones are denoted by “_M” (e.g., “miR-1-1_M”). Table 21 provides miRNA backbone sequences (in DNA format) used in Deep Screen 2. The RNA sequences of the miRNA backbone are provided by converting the “T” nucleotides in the sequences of Table 21 to “U” nucleotides.

TABLE 21
miRNA backbone sequences used in Deep Screen 2

| miR_with | 5′ miR context | | | | | | 3′ miR context |
|---|---|---|---|---|---|---|---|
| suffix | (flanking) | 5′ basal stem | 5p | loop | 3p | 3′ basal stem | (flanking) |
| miR-1-1 | catgcagactgcctgct | TGGG | passenger | TATGGACCTGCTAA | guide | CTCA | ggccgggacctctctegccg |
| | [SEQ ID NO: 1523] | | | GCTA | | | cactgaggggcactccaca |
| | | | | [SEQ ID NO: 1524] | | | ccacgggggccg |
| | | | | | | | [SEQ ID NO: 1525] |
| miR-1-1_M | catgcagactgcctgct | TGGG | passenger | TATGGACCTGCTAA | guide | CTCA | ggccgggacctcttccgccg |
| | [SEQ ID NO: 1526] | | | GCTA | | | cactgaggggcactccaca |
| | | | | [SEQ ID NO: 1527] | | | ccacgggggccg |
| | | | | | | | [SEQ ID NO: 1528] |
| miR-100 | CCCAAAAGAGA | CCTGTTGCCAC | guide | GTATTAGTCCG | passenger | TGTGTCTGTTA | CAATCTCACGGA |
| | GAAGATATTGA | A | | [SEQ ID NO: 1531] | | GG | CCTGGGGCTTTGC |
| | GG | [SEQ ID | | | | [SEQ ID NO: 1532] | TTATATGCC |
| | [SEQ ID NO: 1529] | NO: 1530] | | | | | [SEQ ID NO: 1533] |
| miR-100_M | CCCAAAAGAGA | CCTGTTGCCAC | guide | GTATTAGTCCG | passenger | TGTGTCTGTTA | CtATtcCACGGACC |
| | GAAGATATTGAt | A | | [SEQ ID NO: 1536] | | GG | TGGGGCTTTGCTT |
| | G | [SEQ ID | | | | [SEQ ID NO: 1537] | ATATGCC |
| | [SEQ ID NO: 1534] | NO: 1535] | | | | | [SEQ ID NO: 1538] |
| miR-122 | ggctacagagttt | CCTTAGCAGAG | guide | TGTCTAAACTAT | passenger | TAGCTACTGCT | aatccttccctcgataaatgtc |
| | [SEQ ID NO: 1539] | CTG | | [SEQ ID NO: 1541] | | AGGC | ttggcatcgtttgctttg |
| | | [SEQ ID | | | | [SEQ ID NO: 1542] | [SEQ ID NO: 1543] |
| | | NO: 1540] | | | | | |
| miR-122 M | ggctacagagttt | GCTTAGCAGAG | guide | TGTCTAAACTAT | passenger | TAGCTACTGCT | catccttccctcgataaatgtc |
| | [SEQ ID NO: 1544] | CTG | | [SEQ ID NO: 1546] | | AGGC | ttggcatcgtttgctttg |
| | | [SEQ ID | | | | [SEQ ID NO: 1547] | [SEQ ID NO: 1548] |
| | | NO: 1545] | | | | | |
| miR-124 | TTCCTTCCTCAG | AGGCCTCTCTC | passenger | ATTTAAATGTCCAT | guide | GAATGGGGCTG | GCTGAGCACCGT |
| | GAGAA | [SEQ ID | | ACAAT | | [SEQ ID NO: 1552] | GGGTCGGCGAGG |
| | [SEQ ID NO: 1549] | NO: 1550] | | [SEQ ID NO: 1551] | | | GCCCGCCAagga |
| | | | | | | | [SEQ ID NO: 1553] |
| miR-124 M | TTCCTTCCTCAG | tGGCCTCTCTC | passenger | ATTTAAATGTCCAT | guide | GAATGGGGCTt | aCTGccgcaCGTGG |
| | GAGAA | [SEQ ID | | ACAAT | | [SEQ ID NO: 1557] | GTCGGCGAGGGC |
| | [SEQ ID NO: 1554] | NO: 1555] | | [SEQ ID NO: 1556] | | | CCGCCAagga |
| | | | | | | | [SEQ ID NO: 1558] |
| miR-128 | ATTTtgcaataattggc | TGAGCTGTTGG | passenger | GAGGTTTACATTTC | guide | TTCAGCTGCTTC | ctggcttctttttactcaggttt |
| | cttgttcc | A | | [SEQ ID NO: 1561] | | [SEQ ID NO: 1562] | ccactgct |
| | [SEQ ID NO: 1559] | [SEQ ID | | | | | [SEQ ID NO: 1563] |
| | | NO: 1560] | | | | | |
| miR-130a | gcagggccggcatgcct | TGCTGCTGGCC | passenger | CTGTCTGCACCTGTC | guide | TGGCCGTGTAG | ctacccagcgctggctgcct |
| | c | A | | ACTAG | | TG | cctcagcattg |
| | [SEQ ID NO: 1564] | [SEQ ID | | [SEQ ID NO: 1566] | | [SEQ ID NO: 1567] | [SEQ ID NO: 1568] |
| | | NO: 1565] | | | | | |
| miR-155E | CTGGAGGCTTG | GGGCTGTATGC | guide | TTTTGGCCTCTGACT | passenger | CAGGACAAGGC | TTTATCAGCACTC |
| | CTTT | TG | | GA | | CC | ACATGGAACAAA |
| | [SEQ ID NO: 1569] | [SEQ ID | | [SEQ ID NO: 1571] | | [SEQ ID NO: 1572] | TGGCCACCGTG |
| | | NO: 1570] | | | | | [SEQ ID NO: 1573] |
| miR-155M | CCTGGAGGCTT | AGGCTGTATGC | guide | TTTTGGCCACTGACT | passenger | CAGGACACAAG | TGTTACTAGCACT |
| | GCTGA | TG | | GA | | GCC | CACATGGAACAA |
| | [SEQ ID NO: 1574] | [SEQ ID | | [SEQ ID NO: 1576] | | [SEQ ID NO: 1577] | ATGGCCACC |
| | | NO: 15751 | | | | | [SEQ ID NO: 1578] |
| miR-138-2 | gccggcggagttctggta | CGTTGCTGC | guide | GACGAGCAGCGCAT | passenger | GTTGCATCA | tacccatcctctccaggcga |
| | t | | | CCTCTTACCC | | | gcctcgtgggaccGG |
| | [SEQ ID NO: 1579] | | | [SEQ ID NO: 1580] | | | [SEQ ID NO: 1581] |
| miR-144 | TCAAGCCATGC | TGGGGCCCTGG | passenger | AGTTTGCGATGAGA | guide | AGTCCGGGCAC | AGCTCTGGAGCC |
| | TTCCTGTGCCCC | CT | | CAC | | CCCC | TGACAAGGAggaca |
| | CAG | [SEQ ID | | [SEQ ID NO: 1584] | | [SEQ ID NO: 1585] | [SEQ ID NO: 1586] |
| | [SEQ ID NO: 1582] | NO: 1583] | | | | | |
| miR-190a | GAGCTCAGTCA | TGCAGGCCTCT | guide | TGTTATTTAATCCA | passenger | CTACAGTGTCT | CTGTCTCCGGGG |
| | AACCTGGATGC | GTG | | [SEQ ID NO: 1589] | | TGCC | GTTCCTAATAAA |
| | CTTTTC | [SEQ ID | | | | [SEQ ID NO: 1590] | G |
| | [SEQ ID NO: 1587] | NO: 1588] | | | | | [SEQ ID NO: 1591] |
| miR-190a_ | GAGCTCAGTCA | TGCAGGCgTCT | guide | TGTTATTTAATCCA | passenger | CTACAGTCTCTT | CTGTCTCCGGGG |
| M | AACCTGGATGC | GTG | | [SEQ ID NO: 1594] | | GCC | GTTCCTAATAAA |
| | CTTTTC | [SEQ ID | | | | [SEQ ID NO: 1595] | G |
| | [SEQ ID NO: 1592] | NO: 15931 | | | | | SEQ ID NO: 1596] |
| miR-132 | GCCGTCCGCGC | CCGCCCCCGCG | Passenger | CTGTGGGAACTGGA | guide | CCCCGCAGCAC | CGCGCCACGCCG |
| | GCC | TCTCCAGGG | | GG | | GCCCACGCGC | CGCCCCGAGCC |
| | [SEQ ID NO: 1597] | [SEQ ID | | [SEQ ID NO: 1599] | | [SEQ ID NO: 1600] | [SEQ ID NO: 1601] |
| | | NO: 1598] | | | | | |
| miR-451a | GCTCTCTGCTC | CTTGGGAATGG | guide | None | passenger | TCTTGCTATACC | AAACGTGCCAGG |
| | AGCCTGTCACA | CAAGG | | | | CAGA | AAGAGAACTCAG |
| | ACCTACTGACT | [SEQ ID | | | | [SEQ ID NO: 1604] | [SEQ ID NO: 1605] |
| | GCCAGGGCA | NO: 1603] | | | | | |
| | [SEQ ID NO: 1602] | | | | | | |
| miR-223 | TCCCCACAGAA | CCTGGCCTCCT | passenger | CTCCATGTGGTAGA | guide | AGTGCGGCACA | CTCTAGGCC |
| | GCTCTTGG | GCAGTGCCACG | | G | | TGCTTACCAG | |
| | [SEQ ID NO: 1606] | CT | | [SEQ ID NO: 1608] | | [SEQ ID NO: 1609] | |
| | | [SEQ ID | | | | | |
| | | NO: 1607] | | | | | |
| miR-16-2 | TTATGTTTGGAT | GTTCCACTC | guide | TAGTGAAATATATA | passenger | TAGTGTGAC | AGGGATACAGCA |
| | GAACTGACATA | | | TTAAA | | | ACTATTTTATCAA |
| | CTT | | | [SEQ ID NO: 1611] | | | TTGTTT |
| | [SEQ ID NO: 1610] | | | | | | [SEQ ID NO: 1612] |

Oligonucleotides were designed that embedded the guide sequences described in Table 19 into miRNA backbones, using flanking sequences as defined in Table 21, and with passenger sequences defined by the rules in Table 8. For example, an artificial miRNA with miR-100 backbone in DNA format for insertion into a transfer plasmid may be designed comprising from 5′ to 3′:5′ miR context (flanking) sequence of SEQ ID NO:1529; 5′ basal stem sequence of SEQ ID NO:1530; desired guide sequence; loop sequence of SEQ ID NO:1531; desired passenger sequence designed according to the rules in Table 8; 3′ basal stem sequence of SEQ ID NO:1532; and 3′ miR context (flanking) sequence of SEQ ID NO:1533. The artificial miRNA in RNA format may be obtained by converting the “T” nucleotides in these sequences to “U” nucleotides. The pooled library oligonucleotides were cloned into a lentiviral plasmid pLVX-EF1A-miR-CMV-Puro (5′ LTR to 3′ LTR sequence comprises the nucleotide sequence of SEQ ID:1613) with an EF1alpha promoter to express the amiR, and a CMV promoter to express a PuroR selection marker. The artificial miRNA oligonucleotide may be inserted at nucleotides 3126-3263 of SEQ ID NO:1613. After packaging the library in the plasmid, library composition was assessed by sequencing, and it was noted that the abundance of miRs embedded in the miR-16-2 backbone was in general substantially less than other backbones. One potential explanation would be that during library amplification—when all library elements undergo PCR amplification—elements including the miR-16-2 backbone are amplified less efficiently than other backbones. This could perhaps be because of the strong DNA hairpin that forms with the miR-16-2 backbone. Due to the low number of miR-16-2 backbone elements remaining in the library, counts of miR-16-2 containing guides were low and therefore noisy, and not included in further analyses.

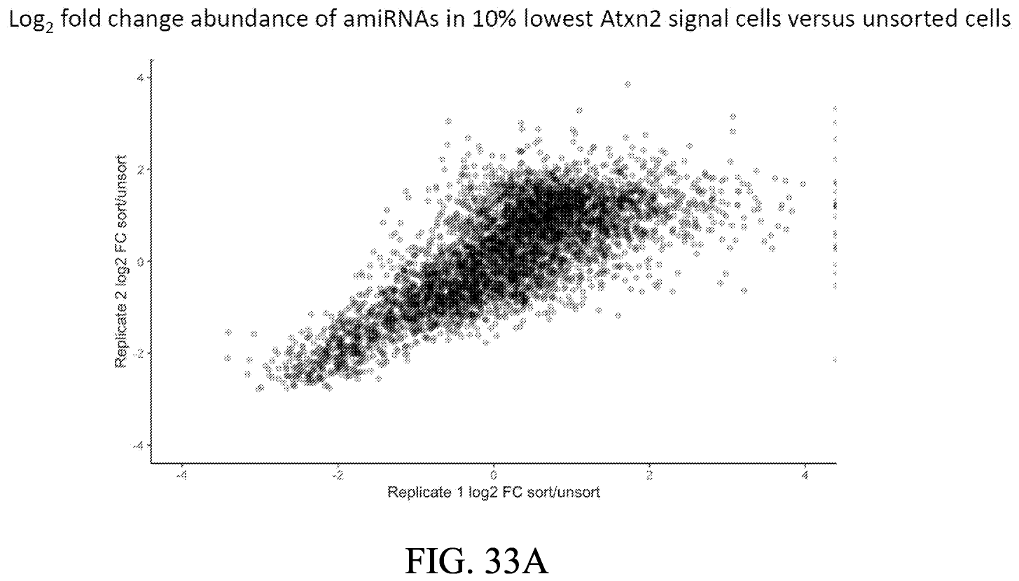

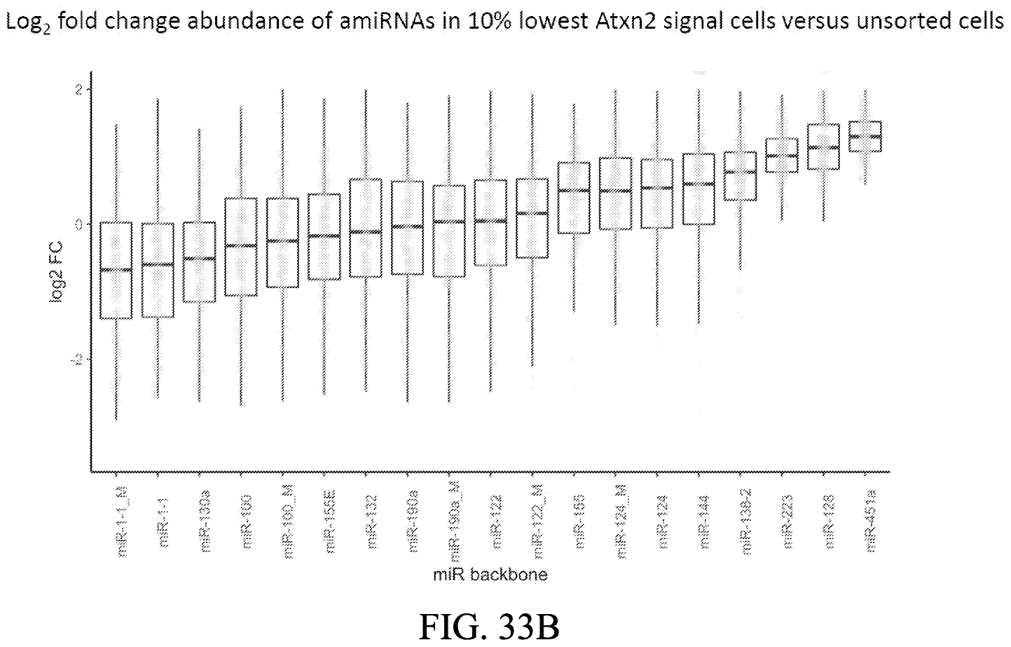

After cloning, packaging, and execution of screen (see methods), sequencing data were analyzed essentially as for Deep Screen 1. Abundance of library elements were calculated by number of sequencing reads exactly matching input library elements. In this screen no baseline subtraction was done for either ATXN2 levels or for dropout. FIG. 33A shows a scatterplot plotting the correspondence in the ATXN2 knockdown metric for two screen replicates against each other. In this case what is plotted is the ratio of abundance of sequence reads for guide elements in the 10% low-ATXN2 signal sort bin versus an unsorted sort bin. There is good correspondence for elements that have low ratios for unsorted/10% low-ATXN2 signal—that is, elements that induce ATXN2 depletion—but there is less correspondence for elements with similar abundance in the unsorted and 10% unsorted bin.

FIG. 33B shows boxplots of knockdown performance of miRs embedded in the shown backbones; Table 22 shows the median and 95^{th} percentiles of performance. By this metric, some of the top performing miRs, as measured by median performing miRNA, were miR-1-1_M, miR1-1, miR-130a, miR-100, and miR-100_M. It is noted, however, that there were top miRNAs in each of these backbones that, as measured by this assay (ratio of counts of guides in a low-Atxn2 sorted pool versus guides in unsorted cells), performed similarly across miR backbones. Therefore, this assay made available multiple miR backbones with strong performance. This was likely due to good processing of the artificial pri-miRNA by the microprocessor and dicer complexes.

TABLE 22
Performance of miRNAs Across miRNA Backbones

| | miR_with_suffix | median | ninety_fifth |
|---|---|---|---|
| 1 | miR-1-1_M | −0.6772802 | −2.2788937 |
| 2 | miR-1-1 | −0.5968477 | −2.1875722 |
| 3 | miR-130a | −0.5111669 | −2.157525 |
| 4 | miR-100 | −0.2912209 | −2.0887075 |
| 5 | miR-100_M | −0.2094112 | −1.9758731 |
| 6 | miR-155E | −0.1742495 | −1.8818528 |
| 7 | miR-132 | −0.1144013 | −2.0204587 |
| 8 | miR-190a | −0.030798 | −2.0813007 |
| 9 | miR-190a_M | 0.04198442 | −2.1250904 |
| 10 | miR-122 | 0.05806267 | −1.9039473 |
| 11 | miR-122_M | 0.17311752 | −1.851009 |
| 12 | miR-155M | 0.50609504 | −1.11192 |
| 13 | miR-124_M | 0.55091372 | −0.9016042 |
| 14 | miR-124 | 0.57242321 | −0.95078 |
| 15 | miR-144 | 0.71869783 | −1.3372836 |
| 16 | miR-138-2 | 0.77876566 | −0.8666664 |
| 17 | miR-223 | 1.04743176 | 0.46242634 |

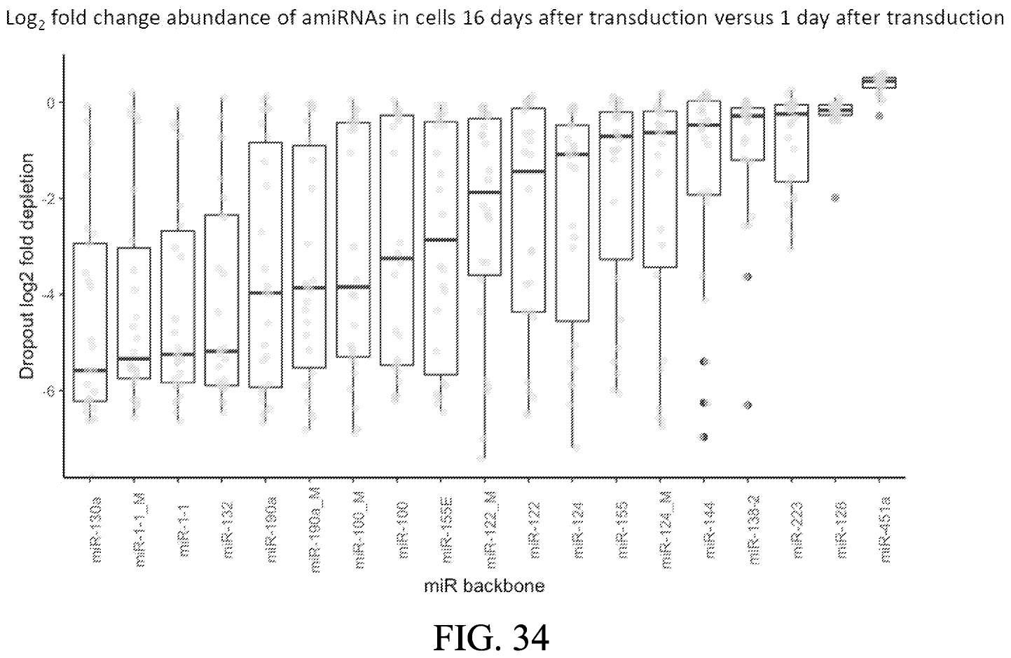

The depletion of elements targeting essential genes was also used as an orthogonal evaluation of miR backbone performance. FIG. 34 shows boxplots of the depletion of elements from the 18-day timepoint versus the 1-day post transduction timepoint. There is a similar ranking of ‘performance’ of the various miR backbones by this metric compared to the ATXN2 knockdown metric. This may be because of the ranking of miR backbones in processing to yield mature amiRNAs.

Table 23 lists the top 100 amiRNAs, ranked by mean enrichment in the ATXN2 low signal sorted cells. The miR backbone, guide sequence, targeting position within the complementary ATXN2 transcript sequence, passenger sequence, and the amiRNA sequence (including the miR backbone, loop, ATXN2 targeting guide and passenger), are provided in both RNA and DNA format. The ‘passenger’ sequence refers to sequence complementary to the guide sequence, but including bulges and mismatches designed according to the rules set forth in Table 8 to mimic endogenous miRNA structure. Note that after processing of the pri-miRNA, the passenger strand will likely initiate 1-3 nt downstream of the nucleotide shown in the table, and include 1-3 nt beyond the last nucleotide listed, derived from the miR cassette. Table 24 lists the top 10 amiRNAs for each miR backbone, excluding low performing backbones. Top amiRNAs were ranked by mean enrichment of sequence counts of the given amiR constructs in the ATXN2 low signal sorted cells. The miR backbone, guide sequence, targeting position within the complementary ATXN2 sequence, passenger sequence, and the amiRNA sequence are provided in RNA and DNA format.

TABLE 23
Top 100 amiRNAs

| Atxn2 | | Atxn2 | | Guide | Passenger | | Guide | Passenger | |
|---|---|---|---|---|---|---|---|---|---|
| Targeting | miR | low/unsort | T1/T0 | Sequence | Sequence | miR Cassette | Sequence | Sequence | miR Cassette |
| Position | Backbone | log2 FC | log2 FC | (DNA) | (DNA) | (DNA) | (RNA) | (RNA) | (RNA) |
| 2945 | miR-1-1_M | -2.89804 | 0.152222 | TGTAGTAG | TGAGCCAA | CATGCAGACTGC | UGUAGUAG | UGAGCCAA | CAUGCAGACUG |
| | | | | AAGGCTTTG | AGCCTTCTA | CTGCTTGGGTGA | AAGGCUUU | AGCCUUCU | CCUGCUUGGGU |
| | | | | GCTGA | CCGACA | GCCAAAGCCTTC | GGCUGA | ACCGACA | GAGCCAAAGCC |
| | | | | [SEQ ID | [SEQ ID | TACCGACATATG | [SEQ ID | [SEQ ID | UUCUACCGACA |
| | | | | NO: 685] | NO: 1633] | GACCTGCTAAGC | NO: 1213] | NO: 1828] | UAUGGACCUGC |
| | | | | | | TATGTAGTAGAA | (Same guide as | | UAAGCUAUGUA |
| | | | | | | GGCTTTGGCTGA | XD-14860) | | GUAGAAGGCUU |
| | | | | | | CTCAGGCCGGG | | | UGGCUGACUCA |
| | | | | | | ACCTCTTCCGCC | | | GGCCGGGACCU |
| | | | | | | GCACTGAGGGG | | | CUUCCGCCGCA |
| | | | | | | CACTCCACACCA | | | CUGAGGGGCAC |
| | | | | | | CGGGGGCC | | | UCCACACCACG |
| | | | | | | [SEQ ID NO: 1711] | | | GGGGCC |
| | | | | | | | | | [SEQ ID NO: 1908] |
| 3330 | miR-190a | -2.86183 | 0.46905 | TATGCTGAG | CCATTATCA | GAGCTCAGTCA | UAUGCUGA | CCAUUAUC | GAGCUCAGUCA |
| | | | | ACTGATAAT | GTCTCAGCA | AACCTGGATGCC | GACUGAUA | AGUCUCAG | AACCUGGAUGC |
| | | | | GTGG | CC | TTTTCTGCAGGC | AUGUGG | CACC | CUUUUCUGCAG |
| | | | | [SEQ ID | [SEQ ID | CTCTGTGTATGC | [SEQ ID | SEQ ID | GCCUCUGUGUA |
| | | | | NO: 1614] | NO: 1634] | TGAGACTGATA | NO: 1811] | NO: 1829] | UGCUGAGACUG |
| | | | | | | ATGTGGTGTTAT | | | AUAAUGUGGUG |
| | | | | | | TTAATCCACCAT | | | UUAUUUAAUCC |
| | | | | | | TATCAGTCTCAG | | | ACCAUUAUCAG |
| | | | | | | CACCCTACAGTG | | | UCUCAGCACCC |
| | | | | | | TCTTGCCCTGTC | | | UACAGUGUCUU |
| | | | | | | TCCGGGGGTTCC | | | GCCCUGUCUCC |
| | | | | | | TAATAAAG | | | GGGGGUUCCUA |
| | | | | | | [SEQ ID NO: 1712] | | | AUAAAG |
| | | | | | | | | | [SEQ ID NO: 1909] |
| 3043 | miR-144 | -2.80802 | 0.325474 | TTTGGTGCA | CCGGTTTGT | TCAAGCCATGCT | UUUGGUGC | CCGGUUUG | UCAAGCCAUGC |
| | | | | AAACAAAC | TTATGCACC | TCCTGTGCCCCC | AAAACAAA | UUUAUGCA | UUCCUGUGCCC |
| | | | | AGGCT | AAA | AGTGGGGCCCT | CAGGCU | CCAAA | CCAGUGGGGCC |
| | | | | [SEQ ID | [SEQ ID | GGCTCCGGTTTG | [SEQ ID | [SEQ ID | CUGGCUCCGGU |
| | | | | NO: 1615] | NO: 1635] | TTTATGCACCAA | NO: 1812] | NO: 1830] | UUGUUUAUGCA |
| | | | | | | AAGTTTGCGATG | | | CCAAAAGUUUG |
| | | | | | | AGACACTTTGGT | | | CGAUGAGACAC |
| | | | | | | GCAAAACAAAC | | | UUUGGUGCAAA |
| | | | | | | AGGAGTCCGGG | | | ACAAACAGGAG |
| | | | | | | CACCCCCAGCTC | | | UCCGGGCACCC |
| | | | | | | TGGAGCCTGAC | | | CCAGCUCUGGA |
| | | | | | | AAGGAGGACA | | | GCCUGACAAGG |
| | | | | | | [SEQ ID NO: 1713] | | | AGGACA |
| | | | | | | | | | [SEQ ID NO: 1910] |
| 2602 | miR-144 | -2.76068 | -0.25422 | TTTAGTAGT | TCGATGGAT | TCAAGCCATGCT | UUUAGUAG | UCGAUGGA | UCAAGCCAUGC |
| | | | | TGATCCATA | CATACTACT | TCCTGTGCCCCC | UUGAUCCA | UCAUACUA | UUCCUGUGCCC |
| | | | | GATT | AAA | AGTGGGGCCCT | UAGAUU | CUAAA | CCAGUGGGGCC |
| | | | | [SEQ ID | [SEQ ID | GGCTTCGATGGA | [SEQ ID | [SEQ ID | CUGGCUUCGAU |
| | | | | NO: 1616] | NO: 1636] | TCATACTACTAA | NO: 202] | NO: 1831] | GGAUCAUACUA |
| | | | | | | AAGTTTGCGATG | (Same guide as | | CUAAAAGUUUG |
| | | | | | | AGACACTTTAGT | XD-14837) | | CGAUGAGACAC |
| | | | | | | AGTTGATCCATA | | | UUUAGUAGUUG |
| | | | | | | GAAGTCCGGGC | | | AUCCAUAGAAG |
| | | | | | | ACCCCCAGCTCT | | | UCCGGGCACCC |
| | | | | | | GGAGCCTGACA | | | CCAGCUCUGGA |
| | | | | | | AGGAGGACA | | | GCCUGACAAGG |
| | | | | | | [SEQ ID NO: 1714] | | | AGGACA |
| | | | | | | | | | [SEQ ID NO: 1911] |
| 3341 | miR-1-1_M | -2.70967 | 0.193529 | ATAGACAT | GACTCAGC | CATGCAGACTGC | AUAGACAU | GACUCAGC | CAUGCAGACUG |
| | | | | GAGGATGC | ATCCTCATG | CTGCTTGGGGAC | GAGGAUGC | AUCCUCAU | CCUGCUUGGGG |
| | | | | TGAGAC | TGATAT | TCAGCATCCTCA | UGAGAC | GUGAUAU | ACUCAGCAUCC |
| | | | | [SEQ ID | SEQ ID | TGTGATATTATG | [SEQ ID | [SEQ ID | UCAUGUGAUAU |
| | | | | NO: 1617] | NO: 1637] | GACCTGCTAAGC | NO: 1813] | NO: 1832] | UAUGGACCUGC |
| | | | | | | TAATAGACATG | | | UAAGCUAAUAG |
| | | | | | | AGGATGCTGAG | | | ACAUGAGGAUG |
| | | | | | | ACCTCAGGCCG | | | CUGAGACCUCA |
| | | | | | | GGACCTCTTCCG | | | GGCCGGGACCU |
| | | | | | | CCGCACTGAGG | | | CUUCCGCCGCA |
| | | | | | | GGCACTCCACAC | | | CUGAGGGGCAC |
| | | | | | | CACGGGGGCC | | | UCCACACCACG |
| | | | | | | [SEQ ID NO: 1715] | | | GGGGCC |
| | | | | | | | | | [SEQ ID NO: 1912] |
| 3302 | miR-100 | -2.68521 | -0.3818 | TTGAACAA | CCCAAACC | CCCAAAAGAGA | UUGAACAA | CCCAAACC | CCCAAAAGAGA |
| | | | | GGGGCTGA | AGACCCTTG | GAAGATATTGA | GGGGCUGA | AGACCCUU | GAAGAUAUUGA |
| | | | | TTTGGG | CTCAT | GGCCTGTTGCCA | UUUGGG | GCUCAU | GGCCUGUUGCC |
| | | | | [SEQ ID | [SEQ ID | CATTGAACAAG | [SEQ ID | [SEQ ID | ACAUUGAACAA |
| | | | | NO: 688] | NO: 1638] | GGGCTGATTTGG | NO: 1216] | NO: 1833] | GGGGCUGAUUU |
| | | | | | | GGTATTAGTCCG | | | GGGGUAUUAGU |
| | | | | | | CCCAAACCAGA | | | CCGCCCAAACC |
| | | | | | | CCCTTGCTCATT | | | AGACCCUUGCU |
| | | | | | | GTGTCTGTTAGG | | | CAUUGUGUCUG |
| | | | | | | CAATCTCACGGA | | | UUAGGCAAUCU |
| | | | | | | CCTGGGGCTTTG | | | CACGGACCUGG |
| | | | | | | CTTATATGCC | | | GGCUUUGCUUA |
| | | | | | | [SEQ ID NO: 1716] | | | UAUGCC |
| | | | | | | | | | [SEQ ID NO: 1913] |
| 3043 | miR-100 | -2.67985 | -0.0584 | TTTGGTGCA | AGCCTGCTT | CCCAAAAGAGA | UUUGGUGC | AGCCUGCU | CCCAAAAGAGA |
| | | | | AAACAAAC | GGTTTGCAA | GAAGATATTGA | AAAACAAA | UGGUUUGC | GAAGAUAUUGA |
| | | | | AGGCT | CAAT | GGCCTGTTGCCA | CAGGCU | AACAAU | GGCCUGUUGCC |
| | | | | [SEQ ID | [SEQ ID | CATTTGGTGCAA | [SEQ ID | [SEQ ID | ACAUUUGGUGC |
| | | | | NO: 1615] | NO: 1639] | AACAAACAGGC | NO: 1812] | NO: 1834] | AAAACAAACAG |
| | | | | | | TGTATTAGTCCG | | | GCUGUAUUAGU |
| | | | | | | AGCCTGCTTGGT | | | CCGAGCCUGCU |
| | | | | | | TTGCAACAATTG | | | UGGUUUGCAAC |
| | | | | | | TGTCTGTTAGGC | | | AAUUGUGUCUG |
| | | | | | | AATCTCACGGAC | | | UUAGGCAAUCU |
| | | | | | | CTGGGGCTTTGC | | | CACGGACCUGG |
| | | | | | | TTATATGCC | | | GGCUUUGCUUA |
| | | | | | | [SEQ ID NO: 1717] | | | UAUGCC |
| | | | | | | | | | [SEQ ID NO: 1914] |
| 1755 | miR-100 | -2.66915 | -0.04161 | TCGGGTTGA | CACACTCCA | CCCAAAAGAGA | UCGGGUUG | CACACUCC | CCCAAAAGAGA |
| | | | | AATCTGAA | GCTTTCAAA | GAAGATATTGA | AAAUCUGA | AGCUUUCA | GAAGAUAUUGA |
| | | | | GTGTG | CCGT | GGCCTGTTGCCA | AGUGUG | AACCGU | GGCCUGUUGCC |
| | | | | [SEQ ID | [SEQ ID | CATCGGGTTGAA | [SEQ ID | [SEQ ID | ACAUCGGGUUG |
| | | | | NO: 657] | NO: 1640] | ATCTGAAGTGTG | NO: 1185] | NO: 1835] | AAAUCUGAAGU |
| | | | | | | GTATTAGTCCGC | | | GUGGUAUUAGU |
| | | | | | | ACACTCCAGCTT | | | CCGCACACUCC |
| | | | | | | TCAAACCGTTGT | | | AGCUUUCAAAC |
| | | | | | | GTCTGTTAGGCA | | | CGUUGUGUCUG |
| | | | | | | ATCTCACGGACC | | | UUAGGCAAUCU |
| | | | | | | TGGGGCTTTGCT | | | CACGGACCUGG |
| | | | | | | TATATGCC | | | |
| | | | | | | [SEQ ID NO: 1718] | | | GGCUUUGCUUA |
| | | | | | | | | | UAUGCC |
| | | | | | | | | | [SEQ ID NO: 1915] |
| 3272 | miR-100 | -2.65898 | -0.1606 | AGGACTGT | GCAATACG | CCCAAAAGAGA | AGGACUGU | GCAAUACG | CCCAAAAGAGA |
| | | | | AGGCAACA | TTTCCTACA | GAAGATATTGA | AGGCAACA | UUUCCUAC | GAAGAUAUUGA |
| | | | | TATTGC | ATCCA | GGCCTGTTGCCA | UAUUGC | AAUCCA | GGCCUGUUGCC |
| | | | | [SEQ ID | [SEQ ID | CAAGGACTGTA | [SEQ ID | [SEQ ID | ACAAGGACUGU |
| | | | | NO: 1618] | NO: 1641] | GGCAACATATTG | NO: 1814] | NO: 1836] | AGGCAACAUAU |
| | | | | | | CGTATTAGTCCG | | | UGCGUAUUAGU |
| | | | | | | GCAATACGTTTC | | | CCGGCAAUACG |
| | | | | | | CTACAATCCATG | | | UUUCCUACAAU |
| | | | | | | TGTCTGTTAGGC | | | CCAUGUGUCUG |
| | | | | | | AATCTCACGGAC | | | UUAGGCAAUCU |
| | | | | | | CTGGGGCTTTGC | | | CACGGACCUGG |
| | | | | | | TTATATGCC | | | GGCUUUGCUUA |
| | | | | | | [SEQ ID NO: 1719] | | | UAUGCC |
| | | | | | | | | | [SEQ ID NO: 1916] |
| 967 | miR- | -2.64307 | 0.113083 | ACTGATGTA | TGGCATATA | GAGCTCAGTCA | ACUGAUGU | UGGCAUAU | GAGCUCAGUCA |
| | 190a_M | | | AGTATATG | CTTACATCA | AACCTGGATGCC | AAGUAUAU | ACUUACAU | AACCUGGAUGC |
| | | | | AACCA | AG | TTTTCTGCAGGC | GAACCA | CAAG | CUUUUCUGCAG |
| | | | | [SEQ ID | [SEQ ID | GTCTGTGACTGA | [SEQ ID | [SEQ ID | GCGUCUGUGAC |
| | | | | NO: 1619] | NO: 1642] | TGTAAGTATATG | NO: 1815] | NO: 1837] | UGAUGUAAGUA |
| | | | | | | AACCATGTTATT | | | UAUGAACCAUG |
| | | | | | | TAATCCATGGCA | | | UUAUUUAAUCC |
| | | | | | | TATACTTACATC | | | AUGGCAUAUAC |
| | | | | | | AAGCTACAGTCT | | | UUACAUCAAGC |
| | | | | | | CTTGCCCTGTCT | | | UACAGUCUCUU |
| | | | | | | CCGGGGGTTCCT | | | GCCCUGUCUCC |
| | | | | | | AATAAAG | | | GGGGGUUCCUA |
| | | | | | | [SEQ ID NO: 1720] | | | AUAAAG |
| | | | | | | | | | [SEQ ID NO: 1917] |
| 3302 | miR-190a | -2.64186 | 0.124524 | TTGAACAA | CCCATCAGC | GAGCTCAGTCA | UUGAACAA | CCCAUCAG | GAGCUCAGUCA |
| | | | | GGGGCTGA | CCCTTGTTC | AACCTGGATGCC | GGGGCUGA | CCCCUUGU | AACCUGGAUGC |
| | | | | TTTGGG | CC | TTTTCTGCAGGC | UUUGGG | UCCC | CUUUUCUGCAG |
| | | | | [SEQ ID | [SEQ ID | CTCTGTGTTGAA | [SEQ ID | [SEQ ID | GCCUCUGUGUU |
| | | | | NO: 688] | NO: 1643] | CAAGGGGCTGA | NO: 1216] | NO: 1838] | GAACAAGGGGC |
| | | | | | | TTTGGGTGTTAT | | | UGAUUUGGGUG |
| | | | | | | TTAATCCACCCA | | | UUAUUUAAUCC |
| | | | | | | TCAGCCCCTTGT | | | ACCCAUCAGCC |
| | | | | | | TCCCCTACAGTG | | | CCUUGUUCCCC |
| | | | | | | TCTTGCCCTGTC | | | UACAGUGUCUU |
| | | | | | | TCCGGGGGTTCC | | | GCCCUGUCUCC |
| | | | | | | TAATAAAG | | | GGGGGUUCCUA |
| | | | | | | [SEQ ID NO: 1721] | | | AUAAAG |
| | | | | | | | | | [SEQ ID NO: 1918] |
| 3302 | miR-130a | -2.62809 | 0.136636 | TTGAACAA | CGCAAATC | GCAGGGCCGGC | UUGAACAA | CGCAAAUC | GCAGGGCCGGC |
| | | | | GGGGCTGA | AGACCCTTG | ATGCCTCTGCTG | GGGGCUGA | AGACCCUU | AUGCCUCUGCU |
| | | | | TTTGGG | TTCAC | CTGGCCACGCA | UUUGGG | GUUCAC | GCUGGCCACGC |
| | | | | [SEQ ID | [SEQ ID | AATCAGACCCTT | [SEQ ID | [SEQ ID | AAAUCAGACCC |
| | | | | NO: 688] | NO: 1644] | GTTCACCTGTCT | NO: 1216] | NO: 1839] | UUGUUCACCUG |
| | | | | | | GCACCTGTCACT | | | UCUGCACCUGU |
| | | | | | | AGTTGAACAAG | | | CACUAGUUGAA |
| | | | | | | GGGCTGATTTGG | | | CAAGGGGCUGA |
| | | | | | | GTGGCCGTGTAG | | | UUUGGGUGGCC |
| | | | | | | TGCTACCCAGCG | | | GUGUAGUGCUA |
| | | | | | | CTGGCTGCCTCC | | | CCCAGCGCUGG |
| | | | | | | TCAGCATTG | | | CUGCCUCCUCA |
| | | | | | | [SEQ ID NO: 1722] | | | GCAUUG |
| | | | | | | | | | [SEQ ID NO: 1919] |
| 1755 | miR-1-1_M | -2.62482 | 0.169485 | TCGGGTTGA | CTCACTTCA | CATGCAGACTGC | UCGGGUUG | CUCACUUC | CAUGCAGACUG |
| | | | | AATCTGAA | GATTTCAAC | CTGCTTGGGCTC | AAAUCUGA | AGAUUUCA | CCUGCUUGGGC |
| | | | | GTGTG | GACGA | ACTTCAGATTTC | AGUGUG | ACGACGA | UCACUUCAGAU |
| | | | | [SEQ ID | [SEQ ID | AACGACGATAT | [SEQ ID | [SEQ ID | UUCAACGACGA |
| | | | | NO: 657] | NO: 1645] | GGACCTGCTAA | NO: 1185] | NO: 1840] | UAUGGACCUGC |
| | | | | | | GCTATCGGGTTG | | | UAAGCUAUCGG |
| | | | | | | AAATCTGAAGT | | | GUUGAAAUCUG |
| | | | | | | GTGCTCAGGCCG | | | AAGUGUGCUCA |
| | | | | | | GGACCTCTTCCG | | | GGCCGGGACCU |
| | | | | | | CCGCACTGAGG | | | CUUCCGCCGCA |
| | | | | | | GGCACTCCACAC | | | CUGAGGGGCAC |
| | | | | | | CACGGGGGCC | | | UCCACACCACG |
| | | | | | | [SEQ ID NO: 1723] | | | GGGGCC |
| | | | | | | | | | [SEQ ID NO: 1920] |
| 3302 | miR-100_M | -2.62238 | -0.19177 | TTGAACAA | CCCAAACC | CCCAAAAGAGA | UUGAACAA | CCCAAACC | CCCAAAAGAGA |
| | | | | GGGGCTGA | AGACCCTTG | GAAGATATTGAT | GGGGCUGA | AGACCCUU | GAAGAUAUUGA |
| | | | | TTTGGG | CTCAT | GCCTGTTGCCAC | UUUGGG | GCUCAU | UGCCUGUUGCC |
| | | | | [SEQ ID | [SEQ ID | ATTGAACAAGG | [SEQ ID | [SEQ ID | ACAUUGAACAA |
| | | | | NO: 688] | NO: 1638] | GGCTGATTTGGG | NO: 1216] | NO: 1833] | GGGGCUGAUUU |
| | | | | | | GTATTAGTCCGC | | | GGGGUAUUAGU |
| | | | | | | CCAAACCAGAC | | | CCGCCCAAACC |
| | | | | | | CCTTGCTCATTG | | | AGACCCUUGCU |
| | | | | | | TGTCTGTTAGGC | | | CAUUGUGUCUG |
| | | | | | | TATTCCACGGAC | | | UUAGGCUAUUC |
| | | | | | | CTGGGGCTTTGC | | | CACGGACCUGG |
| | | | | | | TTATATGCC | | | GGCUUUGCUUA |
| | | | | | | [SEQ ID NO: 1724] | | | UAUGCC |
| | | | | | | | | | [SEQ ID NO: 1921] |
| 1755 | miR-122_M | -2.60043 | -0.03541 | TCGGGTTGA | CACACTTCA | GGCTACAGAGTT | UCGGGUUG | CACACUUC | GGCUACAGAGU |
| | | | | AATCTGAA | GACTTCAAC | TGCTTAGCAGAG | AAAUCUGA | AGACUUCA | UUGCUUAGCAG |
| | | | | GTGTG | CATA | CTGTCGGGTTGA | AGUGUG | ACCAUA | AGCUGUCGGGU |
| | | | | [SEQ ID | [SEQ ID | AATCTGAAGTGT | [SEQ ID | [SEQ ID | UGAAAUCUGAA |
| | | | | NO: 657] | NO: 1646] | GTGTCTAAACTA | NO: 1185] | NO: 1841] | GUGUGUGUCUA |
| | | | | | | TCACACTTCAGA | | | AACUAUCACAC |
| | | | | | | CTTCAACCATAT | | | UUCAGACUUCA |
| | | | | | | AGCTACTGCTAG | | | ACCAUAUAGCU |
| | | | | | | GCCATCCTTCCC | | | ACUGCUAGGCC |
| | | | | | | TCGATAAATGTC | | | AUCCUUCCCUC |
| | | | | | | TTGGCATCGTTT | | | GAUAAAUGUCU |
| | | | | | | GCTTTG | | | UGGCAUCGUUU |
| | | | | | | [SEQ ID NO: 1725] | | | GCUUUG |
| | | | | | | | | | [SEQ ID NO: 1922] |
| 1755 | miR-1-1 | -2.5734 | -0.10352 | TCGGGTTGA | CTCACTTCA | CATGCAGACTGC | UCGGGUUG | CUCACUUC | CAUGCAGACUG |
| | | | | AATCTGAA | GATTTCAAC | CTGCTTGGGCTC | AAAUCUGA | AGAUUUCA | CCUGCUUGGGC |
| | | | | GTGTG | GACGA | ACTTCAGATTTC | AGUGUG | ACGACGA | UCACUUCAGAU |
| | | | | [SEQ ID | [SEQ ID | AACGACGATAT | [SEQ ID | [SEQ ID | UUCAACGACGA |
| | | | | NO: 657] | NO: 1645] | GGACCTGCTAA | NO: 1185] | NO: 1840] | UAUGGACCUGC |
| | | | | | | GCTATCGGGTTG | | | UAAGCUAUCGG |
| | | | | | | AAATCTGAAGT | | | GUUGAAAUCUG |
| | | | | | | GTGCTCAGGCCG | | | AAGUGUGCUCA |
| | | | | | | GGACCTCTCTCG | | | GGCCGGGACCU |
| | | | | | | CCGCACTGAGG | | | CUCUCGCCGCA |
| | | | | | | GGCACTCCACAC | | | CUGAGGGGCAC |
| | | | | | | CACGGGGGCC | | | UCCACACCACG |
| | | | | | | [SEQ ID NO: 1726] | | | GGGGCC |
| | | | | | | | | | [SEQ ID NO: 1923] |
| 3302 | miR-1-1_M | -2.57066 | 0.05742 | TTGAACAA | CGCAAATC | CATGCAGACTGC | UUGAACAA | CGCAAAUC | CAUGCAGACUG |
| | | | | GGGGCTGA | AGCCCCTTG | CTGCTTGGGCGC | GGGGCUGA | AGCCCCUU | CCUGCUUGGGC |
| | | | | TTTGGG | TCGCAA | AAATCAGCCCCT | UUUGGG | GUCGCAA | GCAAAUCAGCC |
| | | | | [SEQ ID | [SEQ ID | TGTCGCAATATG | [SEQ ID | [SEQ ID | CCUUGUCGCAA |
| | | | | NO: 688] | NO: 1647] | GACCTGCTAAGC | NO: 1216] | NO: 1842] | UAUGGACCUGC |
| | | | | | | TATTGAACAAG | | | UAAGCUAUUGA |
| | | | | | | GGGCTGATTTGG | | | ACAAGGGGCUG |
| | | | | | | GCTCAGGCCGG | | | AUUUGGGCUCA |
| | | | | | | GACCTCTTCCGC | | | GGCCGGGACCU |
| | | | | | | CGCACTGAGGG | | | CUUCCGCCGCA |
| | | | | | | GCACTCCACACC | | | CUGAGGGGCAC |
| | | | | | | ACGGGGGCC | | | UCCACACCACG |
| | | | | | | [SEQ ID NO: 1727] | | | GGGGCC |
| | | | | | | | | | [SEQ ID NO: 1924] |
| 1755 | miR-130a | -2.56989 | 0.011195 | TCGGGTTGA | CTCACTTCA | GCAGGGCCGGC | UCGGGUUG | CUCACUUC | GCAGGGCCGGC |
| | | | | AATCTGAA | GCTTTCAAT | ATGCCTCTGCTG | AAAUCUGA | AGCUUUCA | AUGCCUCUGCU |
| | | | | GTGTG | TCGC | CTGGCCACTCAC | AGUGUG | AUUCGC | GCUGGCCACUC |
| | | | | [SEQ ID | [SEQ ID | TTCAGCTTTCAA | [SEQ ID | [SEQ ID | ACUUCAGCUUU |
| | | | | NO: 657] | NO: 1648] | TTCGCCTGTCTG | NO: 1185] | NO: 1843] | CAAUUCGCCUG |
| | | | | | | CACCTGTCACTA | | | UCUGCACCUGU |
| | | | | | | GTCGGGTTGAA | | | CACUAGUCGGG |
| | | | | | | ATCTGAAGTGTG | | | UUGAAAUCUGA |
| | | | | | | TGGCCGTGTAGT | | | AGUGUGUGGCC |
| | | | | | | GCTACCCAGCGC | | | GUGUAGUGCUA |
| | | | | | | TGGCTGCCTCCT | | | CCCAGCGCUGG |
| | | | | | | CAGCATTG | | | CUGCCUCCUCA |
| | | | | | | [SEQ ID NO: 1728] | | | GCAUUG |
| | | | | | | | | | [SEQ ID NO: 1925] |
| 3272 | miR-100_M | -2.56927 | -0.01914 | AGGACTGT | GCAATACG | CCCAAAAGAGA | AGGACUGU | GCAAUACG | CCCAAAAGAGA |
| | | | | AGGCAACA | TTTCCTACA | GAAGATATTGAT | AGGCAACA | UUUCCUAC | GAAGAUAUUGA |
| | | | | TATTGC | ATCCA | GCCTGTTGCCAC | UAUUGC | AAUCCA | UGCCUGUUGCC |
| | | | | [SEQ ID | [SEQ ID | AAGGACTGTAG | [SEQ ID | [SEQ ID | ACAAGGACUGU |
| | | | | NO: 1618] | NO: 1641] | GCAACATATTGC | NO: 1814] | NO: 1836] | AGGCAACAUAU |
| | | | | | | GTATTAGTCCGG | | | UGCGUAUUAGU |
| | | | | | | CAATACGTTTCC | | | CCGGCAAUACG |
| | | | | | | TACAATCCATGT | | | UUUCCUACAAU |
| | | | | | | GTCTGTTAGGCT | | | CCAUGUGUCUG |
| | | | | | | ATTCCACGGACC | | | UUAGGCUAUUC |
| | | | | | | TGGGGCTTTGCT | | | CACGGACCUGG |
| | | | | | | TATATGCC | | | GGCUUUGCUUA |
| | | | | | | [SEQ ID NO: 1729] | | | UAUGCC |
| | | | | | | | | | [SEQ ID NO: 1926] |
| 3301 | miR-130a | -2.55451 | -0.07529 | TGAACAAG | TGCCAAATC | GCAGGGCCGGC | UGAACAAG | UGCCAAAU | GCAGGGCCGGC |
| | | | | GGGCTGATT | ATCCCCTTG | ATGCCTCTGCTG | GGGCUGAU | CAUCCCCU | AUGCCUCUGCU |
| | | | | TGGGA | TTCC | CTGGCCATGCCA | UUGGGA | UGUUCC | GCUGGCCAUGC |
| | | | | [SEQ ID | [SEQ ID | AATCATCCCCTT | [SEQ ID | [SEQ ID | CAAAUCAUCCC |
| | | | | NO: 687] | NO: 1649] | GTTCCCTGTCTG | NO: 1215] | NO: 1844] | CUUGUUCCCUG |
| | | | | | | CACCTGTCACTA | | | UCUGCACCUGU |
| | | | | | | GTGAACAAGGG | | | CACUAGUGAAC |
| | | | | | | GCTGATTTGGGA | | | AAGGGGCUGAU |
| | | | | | | TGGCCGTGTAGT | | | UUGGGAUGGCC |
| | | | | | | GCTACCCAGCGC | | | GUGUAGUGCUA |
| | | | | | | TGGCTGCCTCCT | | | CCCAGCGCUGG |
| | | | | | | CAGCATTG | | | CUGCCUCCUCA |
| | | | | | | [SEQ ID NO: 1730] | | | GCAUUG |
| | | | | | | | | | [SEQ ID NO: 1927] |
| 2943 | miR-1-1 | -2.54936 | 0.148183 | TAGTAGAA | TGTCAGCCA | CATGCAGACTGC | UAGUAGAA | UGUCAGCC | CAUGCAGACUG |
| | | | | GGCTTTGGC | AAGCCTTCT | CTGCTTGGGTGT | GGCUUUGG | AAAGCCUU | CCUGCUUGGGU |
| | | | | TGAGA | CCCTA | CAGCCAAAGCC | CUGAGA | CUCCCUA | GUCAGCCAAAG |
| | | | | [SEQ ID | [SEQ ID | TTCTCCCTATAT | [SEQ ID | [SEQ ID | CCUUCUCCCUA |
| | | | | NO: 683] | NO: 1650] | GGACCTGCTAA | NO: 1211] | NO: 1845] | UAUGGACCUGC |
| | | | | | | GCTATAGTAGA | | | UAAGCUAUAGU |
| | | | | | | AGGCTTTGGCTG | | | AGAAGGCUUUG |
| | | | | | | AGACTCAGGCC | | | GCUGAGACUCA |
| | | | | | | GGGACCTCTCTC | | | GGCCGGGACCU |
| | | | | | | GCCGCACTGAG | | | CUCUCGCCGCA |
| | | | | | | GGGCACTCCAC | | | CUGAGGGGCAC |
| | | | | | | ACCACGGGGGC | | | UCCACACCACG |
| | | | | | | C | | | GGGGCC |
| | | | | | | [SEQ ID NO: 1731] | | | [SEQ ID NO: 1928] |
| 3338 | miR-1-1 | -2.52716 | -0.2585 | TACATGAG | TGAGTCTCA | CATGCAGACTGC | UACAUGAG | UGAGUCUC | CAUGCAGACUG |
| | | | | GATGCTGA | GCATCCTCA | CTGCTTGGGTGA | GAUGCUGA | AGCAUCCU | CCUGCUUGGGU |
| | | | | GACTGA | CGGTA | GTCTCAGCATCC | GACUGA | CACGGUA | GAGUCUCAGCA |
| | | | | [SEQ ID | [SEQ ID | TCACGGTATATG | [SEQ ID | [SEQ ID | UCCUCACGGUA |
| | | | | NO: 1620] | NO: 1651] | GACCTGCTAAGC | NO: 314] | NO: 1846] | UAUGGACCUGC |
| | | | | | | TATACATGAGG | (Same guide as | | UAAGCUAUACA |
| | | | | | | ATGCTGAGACTG | XD-14893) | | UGAGGAUGCUG |
| | | | | | | ACTCAGGCCGG | | | AGACUGACUCA |
| | | | | | | GACCTCTCTCGC | | | GGCCGGGACCU |
| | | | | | | CGCACTGAGGG | | | CUCUCGCCGCA |
| | | | | | | GCACTCCACACC | | | CUGAGGGGCAC |
| | | | | | | ACGGGGGCC | | | UCCACACCACG |
| | | | | | | [SEQ ID NO: 1732] | | | GGGGCC |
| | | | | | | | | | [SEQ ID NO: 1929] |
| 3302 | miR- | -2.52395 | 0.249656 | TTGAACAA | CCCATCAGC | GAGCTCAGTCA | UUGAACAA | CCCAUCAG | GAGCUCAGUCA |
| | 190a_M | | | GGGGCTGA | CCCTTGTTC | AACCTGGATGCC | GGGGCUGA | CCCCUUGU | AACCUGGAUGC |
| | | | | TTTGGG | CC | TTTTCTGCAGGC | UUUGGG | UCCC | CUUUUCUGCAG |
| | | | | [SEQ ID | [SEQ ID | GTCTGTGTTGAA | [SEQ ID | [SEQ ID | GCGUCUGUGUU |
| | | | | NO: 688] | NO: 1643] | CAAGGGGCTGA | NO: 1216] | NO: 1838] | GAACAAGGGGC |
| | | | | | | TTTGGGTGTTAT | | | UGAUUUGGGUG |
| | | | | | | TTAATCCACCCA | | | UUAUUUAAUCC |
| | | | | | | TCAGCCCCTTGT | | | ACCCAUCAGCC |
| | | | | | | TCCCCTACAGTC | | | CCUUGUUCCCC |
| | | | | | | TCTTGCCCTGTC | | | UACAGUCUCUU |
| | | | | | | TCCGGGGGTTCC | | | GCCCUGUCUCC |
| | | | | | | TAATAAAG | | | GGGGGUUCCUA |
| | | | | | | [SEQ ID NO: 1733] | | | AUAAAG |
| | | | | | | | | | [SEQ ID NO: 1930] |
| 3302 | miR-155E | -2.52291 | 0.018122 | TTGAACAA | CCCAAATC | CTGGAGGCTTGC | UUGAACAA | CCCAAAUC | CUGGAGGCUUG |
| | | | | GGGGCTGA | GCCCTTGTT | TTTGGGCTGTAT | GGGGCUGA | GCCCUUGU | CUUUGGGCUGU |
| | | | | TTTGGG | CAA | GCTGTTGAACAA | UUUGGG | UCAA | AUGCUGUUGAA |
| | | | | [SEQ ID | [SEQ ID | GGGGCTGATTTG | [SEQ ID | [SEQ ID | CAAGGGGCUGA |
| | | | | NO: 688] | NO: 1652] | GGTTTTGGCCTC | NO: 1216] | NO: 1847] | UUUGGGUUUUG |
| | | | | | | TGACTGACCCAA | | | GCCUCUGACUG |
| | | | | | | ATCGCCCTTGTT | | | ACCCAAAUCGC |
| | | | | | | CAACAGGACAA | | | CCUUGUUCAAC |
| | | | | | | GGCCCTTTATCA | | | AGGACAAGGCC |
| | | | | | | GCACTCACATGG | | | CUUUAUCAGCA |
| | | | | | | AACAAATGGCC | | | CUCACAUGGAA |
| | | | | | | ACCGTGGG | | | CAAAUGGCCAC |
| | | | | | | [SEQ ID NO: 1734] | | | CGUGGG |
| | | | | | | | | | [SEQ ID NO: 1931] |
| 3302 | miR-1-1 | -2.51939 | -0.06675 | TTGAACAA | CGCAAATC | CATGCAGACTGC | UUGAACAA | CGCAAAUC | CAUGCAGACUG |
| | | | | GGGGCTGA | AGCCCCTTG | CTGCTTGGGCGC | GGGGCUGA | AGCCCCUU | CCUGCUUGGGC |
| | | | | TTTGGG | TCGCAA | AAATCAGCCCCT | UUUGGG | GUCGCAA | GCAAAUCAGCC |
| | | | | [SEQ ID | [SEQ ID | TGTCGCAATATG | [SEQ ID | [SEQ ID | CCUUGUCGCAA |
| | | | | NO: 688] | NO: 1647] | GACCTGCTAAGC | NO: 1216] | NO: 1842] | UAUGGACCUGC |
| | | | | | | TATTGAACAAG | | | UAAGCUAUUGA |
| | | | | | | GGGCTGATTTGG | | | ACAAGGGGCUG |
| | | | | | | GCTCAGGCCGG | | | AUUUGGGCUCA |
| | | | | | | GACCTCTCTCGC | | | GGCCGGGACCU |
| | | | | | | CGCACTGAGGG | | | CUCUCGCCGCA |
| | | | | | | GCACTCCACACC | | | CUGAGGGGCAC |
| | | | | | | ACGGGGGCC | | | UCCACACCACG |
| | | | | | | [SEQ ID NO: 1735] | | | GGGGCC |
| | | | | | | | | | [SEQ ID NO: 1932] |
| 2586 | miR-155E | -2.5179 | -0.10172 | TAGATTCAG | CCAAGTTCA | CTGGAGGCTTGC | UAGAUUCA | CCAAGUUC | CUGGAGGCUUG |
| | | | | AAGTAGAA | CTCTGAATC | TTTGGGCTGTAT | GAAGUAGA | ACUCUGAA | CUUUGGGCUGU |
| | | | | CTTGG | TA | GCTGTAGATTCA | ACUUGG | UCUA | AUGCUGUAGAU |
| | | | | [SEQ ID | [SEQ ID | GAAGTAGAACT | [SEQ ID | [SEQ ID | UCAGAAGUAGA |
| | | | | NO: 1621] | NO: 1653] | TGGTTTTGGCCT | NO: 1816] | NO: 1848] | ACUUGGUUUUG |
| | | | | | | CTGACTGACCAA | | | GCCUCUGACUG |
| | | | | | | GTTCACTCTGAA | | | ACCAAGUUCAC |
| | | | | | | TCTACAGGACA | | | UCUGAAUCUAC |
| | | | | | | AGGCCCTTTATC | | | AGGACAAGGCC |
| | | | | | | AGCACTCACATG | | | CUUUAUCAGCA |
| | | | | | | GAACAAATGGC | | | CUCACAUGGAA |
| | | | | | | CACCGTGGG | | | CAAAUGGCCAC |
| | | | | | | [SEQ ID NO: 1736] | | | CGUGGG |
| | | | | | | | | | [SEQ ID NO: 1933] |
| 3341 | miR-1-1 | -2.50291 | -0.1115 | ATAGACAT | GACTCAGC | CATGCAGACTGC | AUAGACAU | GACUCAGC | CAUGCAGACUG |
| | | | | GAGGATGC | ATCCTCATG | CTGCTTGGGGAC | GAGGAUGC | AUCCUCAU | CCUGCUUGGGG |
| | | | | TGAGAC | TGATAT | TCAGCATCCTCA | UGAGAC | GUGAUAU | ACUCAGCAUCC |
| | | | | [SEQ ID | [SEQ ID | TGTGATATTATG | [SEQ ID | [SEQ ID | UCAUGUGAUAU |
| | | | | NO: 1617] | NO: 1637] | GACCTGCTAAGC | NO: 1813] | NO: 1832] | UAUGGACCUGC |
| | | | | | | TAATAGACATG | | | UAAGCUAAUAG |
| | | | | | | AGGATGCTGAG | | | ACAUGAGGAUG |
| | | | | | | ACCTCAGGCCG | | | CUGAGACCUCA |
| | | | | | | GGACCTCTCTCG | | | GGCCGGGACCU |
| | | | | | | CCGCACTGAGG | | | CUCUCGCCGCA |
| | | | | | | GGCACTCCACAC | | | CUGAGGGGCAC |
| | | | | | | CACGGGGGCC | | | UCCACACCACG |
| | | | | | | [SEQ ID NO: 1737] | | | GGGGCC |
| | | | | | | | | | [SEQ ID NO: 1934] |
| 1580 | miR-190a | -2.50168 | -0.06321 | ACTGGAATT | AGCTTCAG | GAGCTCAGTCA | ACUGGAAU | AGCUUCAG | GAGCUCAGUCA |
| | | | | TCTCTGAAC | AGAAATTC | AACCTGGATGCC | UUCUCUGA | AGAAAUUC | AACCUGGAUGC |
| | | | | TGCT | CAAG | TTTTCTGCAGGC | ACUGCU | CAAG | CUUUUCUGCAG |
| | | | | [SEQ ID | [SEQ ID | CTCTGTGACTGG | [SEQ ID | [SEQ ID | GCCUCUGUGAC |
| | | | | NO: 1622] | NO: 1654] | AATTTCTCTGAA | NO: 1817] | NO: 1849] | UGGAAUUUCUC |
| | | | | | | CTGCTTGTTATT | | | UGAACUGCUUG |
| | | | | | | TAATCCAAGCTT | | | UUAUUUAAUCC |
| | | | | | | CAGAGAAATTC | | | AAGCUUCAGAG |
| | | | | | | CAAGCTACAGT | | | AAAUUCCAAGC |
| | | | | | | GTCTTGCCCTGT | | | UACAGUGUCUU |
| | | | | | | CTCCGGGGGTTC | | | GCCCUGUCUCC |
| | | | | | | CTAATAAAG | | | GGGGGUUCCUA |
| | | | | | | [SEQ ID NO: 1738] | | | AUAAAG |
| | | | | | | | | | [SEQ ID NO: 1935] |
| 3332 | miR-122 | -2.50104 | -0.209 | AGGATGCT | ACATTATCA | GGCTACAGAGTT | AGGAUGCU | ACAUUAUC | GGCUACAGAGU |
| | | | | GAGACTGA | GTATCAGC | TCCTTAGCAGAG | GAGACUGA | AGUAUCAG | UUCCUUAGCAG |
| | | | | TAATGT | ATAAT | CTGAGGATGCTG | UAAUGU | CAUAAU | AGCUGAGGAUG |
| | | | | [SEQ ID | [SEQ ID | AGACTGATAAT | [SEQ ID | [SEQ ID | CUGAGACUGAU |
| | | | | NO: 1623] | NO: 1655] | GTTGTCTAAACT | NO: 1818] | NO: 1850] | AAUGUUGUCUA |
| | | | | | | ATACATTATCAG | | | AACUAUACAUU |
| | | | | | | TATCAGCATAAT | | | AUCAGUAUCAG |
| | | | | | | TAGCTACTGCTA | | | CAUAAUUAGCU |
| | | | | | | GGCAATCCTTCC | | | ACUGCUAGGCA |
| | | | | | | CTCGATAAATGT | | | AUCCUUCCCUC |
| | | | | | | CTTGGCATCGTT | | | GAUAAAUGUCU |
| | | | | | | TGCTTTG | | | UGGCAUCGUUU |
| | | | | | | [SEQ ID NO: 1739] | | | GCUUUG |
| | | | | | | | | | [SEQ ID NO: 1936] |
| 3133 | miR-100_M | -2.49525 | 0.238806 | TATGTCTTG | CAGTGACTC | CCCAAAAGAGA | UAUGUCUU | CAGUGACU | CCCAAAAGAGA |
| | | | | GCTTGATTC | ACGCCAAG | GAAGATATTGAT | GGCUUGAU | CACGCCAA | GAAGAUAUUGA |
| | | | | ACTG | CCATT | GCCTGTTGCCAC | UCACUG | GCCAUU | UGCCUGUUGCC |
| | | | | [SEQ ID | [SEQ ID | ATATGTCTTGGC | [SEQ ID | [SEQ ID | ACAUAUGUCUU |
| | | | | NO: 1624] | NO: 1656] | TTGATTCACTGG | NO: 1819] | NO: 1851] | GGCUUGAUUCA |
| | | | | | | TATTAGTCCGCA | | | CUGGUAUUAGU |
| | | | | | | GTGACTCACGCC | | | CCGCAGUGACU |
| | | | | | | AAGCCATTTGTG | | | CACGCCAAGCC |
| | | | | | | TCTGTTAGGCTA | | | AUUUGUGUCUG |
| | | | | | | TTCCACGGACCT | | | UUAGGCUAUUC |
| | | | | | | GGGGCTTTGCTT | | | CACGGACCUGG |
| | | | | | | ATATGCC | | | GGCUUUGCUUA |
| | | | | | | [SEQ ID NO: 1740] | | | UAUGCC |
| | | | | | | | | | [SEQ ID NO: 1937] |
| 3341 | miR-122 | -2.4857 | 0.187025 | ATAGACAT | GTCTCAGCA | GGCTACAGAGTT | AUAGACAU | GUCUCAGC | GGCUACAGAGU |
| | | | | GAGGATGC | TCATCATGT | TCCTTAGCAGAG | GAGGAUGC | AUCAUCAU | UUCCUUAGCAG |
| | | | | TGAGAC | CGCT | CTGATAGACATG | UGAGAC | GUCGCU | AGCUGAUAGAC |
| | | | | [SEQ ID | [SEQ ID | AGGATGCTGAG | [SEQ ID | [SEQ ID | AUGAGGAUGCU |
| | | | | NO: 1617] | NO: 1657] | ACTGTCTAAACT | NO: 1813] | NO: 1852] | GAGACUGUCUA |
| | | | | | | ATGTCTCAGCAT | | | AACUAUGUCUC |
| | | | | | | CATCATGTCGCT | | | AGCAUCAUCAU |
| | | | | | | TAGCTACTGCTA | | | GUCGCUUAGCU |
| | | | | | | GGCAATCCTTCC | | | ACUGCUAGGCA |
| | | | | | | CTCGATAAATGT | | | AUCCUUCCCUC |
| | | | | | | CTTGGCATCGTT | | | GAUAAAUGUCU |
| | | | | | | TGCTTTG | | | UGGCAUCGUUU |
| | | | | | | [SEQ ID NO: 1741] | | | GCUUUG |
| | | | | | | | | | [SEQ ID NO: 1938] |
| 3341 | miR-132 | -2.48196 | 0.14613 | ATAGACAT | GCCTCAGC | GCCGTCCGCGCG | AUAGACAU | GCCUCAGC | GCCGUCCGCGC |
| | | | | GAGGATGC | ATCATAATG | CCCCGCCCCCGC | GAGGAUGC | AUCAUAAU | GCCCCGCCCCC |
| | | | | TGAGAC | TCTAT | GTCTCCAGGGGC | UGAGAC | GUCUAU | GCGUCUCCAGG |
| | | | | [SEQ ID | [SEQ ID | CTCAGCATCATA | [SEQ ID | [SEQ ID | GGCCUCAGCAU |
| | | | | NO: 1617] | NO: 1658] | ATGTCTATCTGT | NO: 1813] | NO: 1853] | CAUAAUGUCUA |
| | | | | | | GGGAACTGGAG | | | UCUGUGGGAAC |
| | | | | | | GATAGACATGA | | | UGGAGGAUAGA |
| | | | | | | GGATGCTGAGA | | | CAUGAGGAUGC |
| | | | | | | CCCCCGCAGCAC | | | UGAGACCCCCG |
| | | | | | | GCCCACGCGCC | | | CAGCACGCCCA |
| | | | | | | GCGCCACGCCG | | | CGCGCCGCGCC |
| | | | | | | CGCCCCGAGCC | | | ACGCCGCGCCC |
| | | | | | | [SEQ ID NO: 1742] | | | CGAGCC |
| | | | | | | | | | [SEQ ID NO: 1939] |
| 3330 | miR-1-1_M | -2.47751 | 0.392579 | TATGCTGAG | CGACATTAT | CATGCAGACTGC | UAUGCUGA | CGACAUUA | CAUGCAGACUG |
| | | | | ACTGATAAT | CAGTCTCAG | CTGCTTGGGCGA | GACUGAUA | UCAGUCUC | CCUGCUUGGGC |
| | | | | GTGG | GAATA | CATTATCAGTCT | AUGUGG | AGGAAUA | GACAUUAUCAG |
| | | | | [SEQ ID | [SEQ ID | CAGGAATATAT | [SEQ ID | [SEQ ID | UCUCAGGAAUA |
| | | | | NO: 1614] | NO: 1659] | GGACCTGCTAA | NO: 1811] | NO: 1854] | UAUGGACCUGC |
| | | | | | | GCTATATGCTGA | | | UAAGCUAUAUG |
| | | | | | | GACTGATAATGT | | | CUGAGACUGAU |
| | | | | | | GGCTCAGGCCG | | | AAUGUGGCUCA |
| | | | | | | GGACCTCTTCCG | | | GGCCGGGACCU |
| | | | | | | CCGCACTGAGG | | | CUUCCGCCGCA |
| | | | | | | GGCACTCCACAC | | | CUGAGGGGCAC |
| | | | | | | CACGGGGGCC | | | UCCACACCACG |
| | | | | | | [SEQ ID NO: 1743] | | | GGGGCC |
| | | | | | | | | | [SEQ ID NO: 1940] |
| 3255 | miR-144 | -2.47679 | -0.08595 | ATTGCGTGG | ACAAGCTT | TCAAGCCATGCT | AUUGCGUG | ACAAGCUU | UCAAGCCAUGC |
| | | | | AGTAAGCT | ACTGCCAC | TCCTGTGCCCCC | GAGUAAGC | ACUGCCAC | UUCCUGUGCCC |
| | | | | GGTGG | GCAAT | AGTGGGGCCCT | UGGUGG | GCAAU | CCAGUGGGGCC |
| | | | | [SEQ ID | [SEQ ID | GGCTACAAGCTT | [SEQ ID | [SEQ ID | CUGGCUACAAG |
| | | | | NO: 617] | NO: 1660] | ACTGCCACGCA | NO: 306] | NO: 1855] | CUUACUGCCAC |
| | | | | | | ATAGTTTGCGAT | (Same guide as | | GCAAUAGUUUG |
| | | | | | | GAGACACATTG | XD-14889) | | CGAUGAGACAC |
| | | | | | | CGTGGAGTAAG | | | AUUGCGUGGAG |
| | | | | | | CTGGTAGTCCGG | | | UAAGCUGGUAG |
| | | | | | | GCACCCCCAGCT | | | UCCGGGCACCC |
| | | | | | | CTGGAGCCTGAC | | | CCAGCUCUGGA |
| | | | | | | AAGGAGGACA | | | GCCUGACAAGG |
| | | | | | | [SEQ ID NO: 1744] | | | AGGACA |
| | | | | | | | | | [SEQ ID NO: 1941] |
| 1755 | miR-100 M | -2.4723 | -0.07055 | TCGGGTTGA | CACACTCCA | CCCAAAAGAGA | UCGGGUUG | CACACUCC | CCCAAAAGAGA |
| | | | | AATCTGAA | GCTTTCAAA | GAAGATATTGAT | AAAUCUGA | AGCUUUCA | GAAGAUAUUGA |
| | | | | GTGTG | CCGT | GCCTGTTGCCAC | AGUGUG | AACCGU | UGCCUGUUGCC |
| | | | | SEQ ID | [SEQ ID | ATCGGGTTGAA | [SEQ ID | [SEQ ID | ACAUCGGGUUG |
| | | | | NO: 657] | NO: 1640] | ATCTGAAGTGTG | NO: 1185] | NO: 1835] | AAAUCUGAAGU |
| | | | | | | GTATTAGTCCGC | | | GUGGUAUUAGU |
| | | | | | | ACACTCCAGCTT | | | CCGCACACUCC |
| | | | | | | TCAAACCGTTGT | | | AGCUUUCAAAC |
| | | | | | | GTCTGTTAGGCT | | | CGUUGUGUCUG |
| | | | | | | ATTCCACGGACC | | | UUAGGCUAUUC |
| | | | | | | TGGGGCTTTGCT | | | CACGGACCUGG |
| | | | | | | TATATGCC | | | GGCUUUGCUUA |
| | | | | | | [SEQ ID NO: 1745] | | | UAUGCC |
| | | | | | | | | | [SEQ ID NO: 1942] |
| 2586 | miR- | -2.46486 | 0.014777 | TAGATTCAG | CCATTCTAC | GAGCTCAGTCA | UAGAUUCA | CCAUUCUA | GAGCUCAGUCA |
| | 190a_M | | | AAGTAGAA | TTCTGAATC | AACCTGGATGCC | GAAGUAGA | CUUCUGAA | AACCUGGAUGC |
| | | | | CTTGG | CC | TTTTCTGCAGGC | ACUUGG | UCCC | CUUUUCUGCAG |
| | | | | SEQ ID | [SEQ ID | GTCTGTGTAGAT | [SEQ ID | [SEQ ID | GCGUCUGUGUA |
| | | | | NO: 1621] | NO: 1661] | TCAGAAGTAGA | NO: 1816] | NO: 1856] | GAUUCAGAAGU |
| | | | | | | ACTTGGTGTTAT | | | AGAACUUGGUG |
| | | | | | | TTAATCCACCAT | | | UUAUUUAAUCC |
| | | | | | | TCTACTTCTGAA | | | ACCAUUCUACU |
| | | | | | | TCCCCTACAGTC | | | UCUGAAUCCCC |
| | | | | | | TCTTGCCCTGTC | | | UACAGUCUCUU |
| | | | | | | TCCGGGGGTTCC | | | GCCCUGUCUCC |
| | | | | | | TAATAAAG | | | GGGGGUUCCUA |
| | | | | | | [SEQ ID NO: 1746] | | | AUAAAG |
| | | | | | | | | | [SEQ ID NO: 1943] |
| 2586 | miR-1-1_M | -2.46317 | 0.179187 | TAGATTCAG | CGAAGTTCT | CATGCAGACTGC | UAGAUUCA | CGAAGUUC | CAUGCAGACUG |
| | | | | AAGTAGAA | ACTTCTGAA | CTGCTTGGGCGA | GAAGUAGA | UACUUCUG | CCUGCUUGGGC |
| | | | | CTTGG | CGCTA | AGTTCTACTTCT | ACUUGG | AACGCUA | GAAGUUCUACU |
| | | | | [SEQ ID | [SEQ ID | GAACGCTATATG | SEQ ID | [SEQ ID | UCUGAACGCUA |
| | | | | NO: 1621] | NO: 1662] | GACCTGCTAAGC | NO: 1816] | NO: 1857] | UAUGGACCUGC |
| | | | | | | TATAGATTCAGA | | | UAAGCUAUAGA |
| | | | | | | AGTAGAACTTG | | | UUCAGAAGUAG |
| | | | | | | GCTCAGGCCGG | | | AACUUGGCUCA |
| | | | | | | GACCTCTTCCGC | | | GGCCGGGACCU |
| | | | | | | CGCACTGAGGG | | | CUUCCGCCGCA |
| | | | | | | GCACTCCACACC | | | CUGAGGGGCAC |
| | | | | | | ACGGGGGCC | | | UCCACACCACG |
| | | | | | | [SEQ ID NO: 1747] | | | GGGGCC |
| | | | | | | | | | [SEQ ID NO: 1944] |
| 3272 | miR-1-1_M | -2.45517 | 0.050153 | AGGACTGT | GGAATATG | CATGCAGACTGC | AGGACUGU | GGAAUAUG | CAUGCAGACUG |
| | | | | AGGCAACA | TTGCCTACA | CTGCTTGGGGGA | AGGCAACA | UUGCCUAC | CCUGCUUGGGG |
| | | | | TATTGC | GCGCCT | ATATGTTGCCTA | UAUUGC | AGCGCCU | GAAUAUGUUGC |
| | | | | SEQ ID | SEQ ID | CAGCGCCTTATG | [SEQ ID | [SEQ ID | CUACAGCGCCU |
| | | | | NO: 1618] | NO: 1663] | GACCTGCTAAGC | NO: 1814] | NO: 1858] | UAUGGACCUGC |
| | | | | | | TAAGGACTGTA | | | UAAGCUAAGGA |
| | | | | | | GGCAACATATTG | | | CUGUAGGCAAC |
| | | | | | | CCTCAGGCCGG | | | AUAUUGCCUCA |
| | | | | | | GACCTCTTCCGC | | | GGCCGGGACCU |
| | | | | | | CGCACTGAGGG | | | CUUCCGCCGCA |
| | | | | | | GCACTCCACACC | | | CUGAGGGGCAC |
| | | | | | | ACGGGGGCC | | | UCCACACCACG |
| | | | | | | [SEQ ID NO: 1748] | | | GGGGCC |
| | | | | | | | | | [SEQ ID NO: 1945] |
| 2943 | miR-1-1_M | -2.45216 | 0.338577 | TAGTAGAA | TGTCAGCCA | CATGCAGACTGC | UAGUAGAA | UGUCAGCC | CAUGCAGACUG |
| | | | | GGCTTTGGC | AAGCCTTCT | CTGCTTGGGTGT | GGCUUUGG | AAAGCCUU | CCUGCUUGGGU |
| | | | | TGAGA | CCCTA | CAGCCAAAGCC | CUGAGA | CUCCCUA | GUCAGCCAAAG |
| | | | | SEQ ID | [SEQ ID | TTCTCCCTATAT | [SEQ ID | [SEQ ID | CCUUCUCCCUA |
| | | | | NO: 683] | NO: 1650] | GGACCTGCTAA | NO: 1211] | NO: 1845] | UAUGGACCUGC |
| | | | | | | GCTATAGTAGA | | | UAAGCUAUAGU |
| | | | | | | AGGCTTTGGCTG | | | AGAAGGCUUUG |
| | | | | | | AGACTCAGGCC | | | GCUGAGACUCA |
| | | | | | | GGGACCTCTTCC | | | GGCCGGGACCU |
| | | | | | | GCCGCACTGAG | | | CUUCCGCCGCA |
| | | | | | | GGGCACTCCAC | | | CUGAGGGGCAC |
| | | | | | | ACCACGGGGGC | | | UCCACACCACG |
| | | | | | | C | | | GGGGCC |
| | | | | | | [SEQ ID NO: 1749] | | | [SEQ ID NO: 1946] |
| 3043 | miR- | -2.44458 | 0.289334 | TTTGGTGCA | AGCGTTTGT | GAGCTCAGTCA | UUUGGUGC | AGCGUUUG | GAGCUCAGUCA |
| | 190a M | | | AAACAAAC | TTTGCACCA | AACCTGGATGCC | AAAACAAA | UUUUGCAC | AACCUGGAUGC |
| | | | | AGGCT | CC | TTTTCTGCAGGC | CAGGCU | CACC | CUUUUCUGCAG |
| | | | | [SEQ ID | [SEQ ID | GTCTGTGTTTGG | [SEQ ID | [SEQ ID | GCGUCUGUGUU |
| | | | | NO: 1615] | NO: 1664] | TGCAAAACAAA | NO: 1812] | NO: 1859] | UGGUGCAAAAC |
| | | | | | | CAGGCTTGTTAT | | | AAACAGGCUUG |
| | | | | | | TTAATCCAAGCG | | | UUAUUUAAUCC |
| | | | | | | TTTGTTTTGCAC | | | AAGCGUUUGUU |
| | | | | | | CACCCTACAGTC | | | UUGCACCACCC |
| | | | | | | TCTTGCCCTGTC | | | UACAGUCUCUU |
| | | | | | | TCCGGGGGTTCC | | | GCCCUGUCUCC |
| | | | | | | TAATAAAG | | | GGGGGUUCCUA |
| | | | | | | [SEQ ID NO: 1750] | | | AUAAAG |
| | | | | | | | | | [SEQ ID NO: 1947] |
| 1578 | miR-100 | -2.43871 | -0.43493 | TGGAATTTC | ACAGCAAT | CCCAAAAGAGA | UGGAAUUU | ACAGCAAU | CCCAAAAGAGA |
| | | | | TCTGAACTG | TCCGAGAA | GAAGATATTGA | CUCUGAAC | UCCGAGAA | GAAGAUAUUGA |
| | | | | CTGT | ACTCCT | GGCCTGTTGCCA | UGCUGU | ACUCCU | GGCCUGUUGCC |
| | | | | [SEQ ID | [SEQ ID | CATGGAATTTCT | [SEQ ID | [SEQ ID | ACAUGGAAUUU |
| | | | | NO: 1626] | NO: 1665] | CTGAACTGCTGT | NO: 1820] | NO: 1860] | CUCUGAACUGC |
| | | | | | | GTATTAGTCCGA | | | UGUGUAUUAGU |
| | | | | | | CAGCAATTCCGA | | | CCGACAGCAAU |
| | | | | | | GAAACTCCTTGT | | | UCCGAGAAACU |
| | | | | | | GTCTGTTAGGCA | | | CCUUGUGUCUG |
| | | | | | | ATCTCACGGACC | | | UUAGGCAAUCU |
| | | | | | | TGGGGCTTTGCT | | | CACGGACCUGG |
| | | | | | | TATATGCC | | | GGCUUUGCUUA |
| | | | | | | [SEQ ID NO: 1751] | | | UAUGCC |
| | | | | | | | | | [SEQ ID NO: 1948] |
| 3133 | miR-122_M | -2.43848 | 0.197681 | TATGTCTTG | CAGTGAAT | GGCTACAGAGTT | UAUGUCUU | CAGUGAAU | GGCUACAGAGU |
| | | | | GCTTGATTC | CAAACCAA | TGCTTAGCAGAG | GGCUUGAU | CAAACCAA | UUGCUUAGCAG |
| | | | | ACTG | GACCGA | CTGTATGTCTTG | UCACUG | GACCGA | AGCUGUAUGUC |
| | | | | [SEQ ID | [SEQ ID | GCTTGATTCACT | [SEQ ID | [SEQ ID | UUGGCUUGAUU |
| | | | | NO: 1624] | NO: 1666] | GTGTCTAAACTA | NO: 1819] | NO: 1861] | CACUGUGUCUA |
| | | | | | | TCAGTGAATCAA | | | AACUAUCAGUG |
| | | | | | | ACCAAGACCGA | | | AAUCAAACCAA |
| | | | | | | TAGCTACTGCTA | | | GACCGAUAGCU |
| | | | | | | GGCCATCCTTCC | | | ACUGCUAGGCC |
| | | | | | | CTCGATAAATGT | | | AUCCUUCCCUC |
| | | | | | | CTTGGCATCGTT | | | GAUAAAUGUCU |
| | | | | | | TGCTTTG | | | UGGCAUCGUUU |
| | | | | | | [SEQ ID NO: 1752] | | | GCUUUG |
| | | | | | | | | | [SEQ ID NO: 1949] |
| 3341 | miR-130a | -2.43802 | 0.12379 | ATAGACAT | GACTCAGC | GCAGGGCCGGC | AUAGACAU | GACUCAGC | GCAGGGCCGGC |
| | | | | GAGGATGC | ATACTCATG | ATGCCTCTGCTG | GAGGAUGC | AUACUCAU | AUGCCUCUGCU |
| | | | | TGAGAC | TTTAC | CTGGCCAGACTC | UGAGAC | GUUUAC | GCUGGCCAGAC |
| | | | | [SEQ ID | [SEQ ID | AGCATACTCATG | [SEQ ID | [SEQ ID | UCAGCAUACUC |
| | | | | NO: 1617] | NO: 1667] | TTTACCTGTCTG | NO: 1813] | NO: 1862] | AUGUUUACCUG |
| | | | | | | CACCTGTCACTA | | | UCUGCACCUGU |
| | | | | | | GATAGACATGA | | | CACUAGAUAGA |
| | | | | | | GGATGCTGAGA | | | CAUGAGGAUGC |
| | | | | | | CTGGCCGTGTAG | | | UGAGACUGGCC |
| | | | | | | TGCTACCCAGCG | | | GUGUAGUGCUA |
| | | | | | | CTGGCTGCCTCC | | | CCCAGCGCUGG |
| | | | | | | TCAGCATTG | | | CUGCCUCCUCA |
| | | | | | | [SEQ ID NO: 1753] | | | GCAUUG |
| | | | | | | | | | [SEQ ID NO: 1950] |
| 3330 | miR-130a | -2.43606 | 0.221874 | TATGCTGAG | CGACATTAT | GCAGGGCCGGC | UAUGCUGA | CGACAUUA | GCAGGGCCGGC |
| | | | | ACTGATAAT | CCGTCTCAG | ATGCCTCTGCTG | GACUGAUA | UCCGUCUC | AUGCCUCUGCU |
| | | | | GTGG | TATC | CTGGCCACGAC | AUGUGG | AGUAUC | GCUGGCCACGA |
| | | | | [SEQ ID | [SEQ ID | ATTATCCGTCTC | [SEQ ID | [SEQ ID | CAUUAUCCGUC |
| | | | | NO: 1614] | NO: 1668] | AGTATCCTGTCT | NO: 1811] | NO: 1863] | UCAGUAUCCUG |
| | | | | | | GCACCTGTCACT | | | UCUGCACCUGU |
| | | | | | | AGTATGCTGAG | | | CACUAGUAUGC |
| | | | | | | ACTGATAATGTG | | | UGAGACUGAUA |
| | | | | | | GTGGCCGTGTAG | | | AUGUGGUGGCC |
| | | | | | | TGCTACCCAGCG | | | GUGUAGUGCUA |
| | | | | | | CTGGCTGCCTCC | | | CCCAGCGCUGG |
| | | | | | | TCAGCATTG | | | CUGCCUCCUCA |
| | | | | | | [SEQ ID NO: 1754] | | | GCAUUG |
| | | | | | | | | | [SEQ ID NO: 1951] |
| 3255 | miR-130a | -2.43262 | 0.01754 | ATTGCGTGG | CGACCAGC | GCAGGGCCGGC | AUUGCGUG | CGACCAGC | GCAGGGCCGGC |
| | | | | AGTAAGCT | TTCCTCCAC | ATGCCTCTGCTG | GAGUAAGC | UUCCUCCA | AUGCCUCUGCU |
| | | | | GGTGG | GTAAC | CTGGCCACGACC | UGGUGG | CGUAAC | GCUGGCCACGA |
| | | | | [SEQ ID | [SEQ ID | AGCTTCCTCCAC | [SEQ ID | [SEQ ID | CCAGCUUCCUC |
| | | | | NO: 617] | NO: 1669] | GTAACCTGTCTG | NO: 306] | NO: 1864] | CACGUAACCUG |
| | | | | | | CACCTGTCACTA | (Same guide as | | UCUGCACCUGU |
| | | | | | | GATTGCGTGGA | XD-14889) | | CACUAGAUUGC |
| | | | | | | GTAAGCTGGTG | | | GUGGAGUAAGC |
| | | | | | | GTGGCCGTGTAG | | | UGGUGGUGGCC |
| | | | | | | TGCTACCCAGCG | | | GUGUAGUGCUA |
| | | | | | | CTGGCTGCCTCC | | | CCCAGCGCUGG |
| | | | | | | TCAGCATTG | | | CUGCCUCCUCA |
| | | | | | | [SEQ ID NO: 1755] | | | GCAUUG |
| | | | | | | | | | [SEQ ID NO: 1952] |
| 3302 | miR-132 | -2.43028 | 0.018802 | TTGAACAA | CACAAATC | GCCGTCCGCGCG | UUGAACAA | CACAAAUC | GCCGUCCGCGC |
| | | | | GGGGCTGA | AGCACATT | CCCCGCCCCCGC | GGGGCUGA | AGCACAUU | GCCCCGCCCCC |
| | | | | TTTGGG | GTTCAA | GTCTCCAGGGCA | UUUGGG | GUUCAA | GCGUCUCCAGG |
| | | | | [SEQ ID | [SEQ ID | CAAATCAGCAC | [SEQ ID | [SEQ ID | GCACAAAUCAG |
| | | | | NO: 688] | NO: 1670] | ATTGTTCAACTG | NO: 1216] | NO: 1865] | CACAUUGUUCA |
| | | | | | | TGGGAACTGGA | | | ACUGUGGGAAC |
| | | | | | | GGTTGAACAAG | | | UGGAGGUUGAA |
| | | | | | | GGGCTGATTTGG | | | CAAGGGGCUGA |
| | | | | | | GCCCCGCAGCA | | | UUUGGGCCCCG |
| | | | | | | CGCCCACGCGCC | | | CAGCACGCCCA |
| | | | | | | GCGCCACGCCG | | | CGCGCCGCGCC |
| | | | | | | CGCCCCGAGCC | | | ACGCCGCGCCC |
| | | | | | | [SEQ ID NO: 1756] | | | CGAGCC |
| | | | | | | | | | [SEQ ID NO: 1953] |
| 3133 | miR-130a | -2.42664 | 0.412413 | TATGTCTTG | CTGTGAATC | GCAGGGCCGGC | UAUGUCUU | CUGUGAAU | GCAGGGCCGGC |
| | | | | GCTTGATTC | ACGCCAAG | ATGCCTCTGCTG | GGCUUGAU | CACGCCAA | AUGCCUCUGCU |
| | | | | ACTG | ATATC | CTGGCCACTGTG | UCACUG | GAUAUC | GCUGGCCACUG |
| | | | | [SEQ ID | [SEQ ID | AATCACGCCAA | [SEQ ID | [SEQ ID | UGAAUCACGCC |
| | | | | NO: 1624] | NO: 1671] | GATATCCTGTCT | NO: 1819] | NO: 1866] | AAGAUAUCCUG |
| | | | | | | GCACCTGTCACT | | | UCUGCACCUGU |
| | | | | | | AGTATGTCTTGG | | | CACUAGUAUGU |
| | | | | | | CTTGATTCACTG | | | CUUGGCUUGAU |
| | | | | | | TGGCCGTGTAGT | | | UCACUGUGGCC |
| | | | | | | GCTACCCAGCGC | | | GUGUAGUGCUA |
| | | | | | | TGGCTGCCTCCT | | | CCCAGCGCUGG |
| | | | | | | CAGCATTG | | | CUGCCUCCUCA |
| | | | | | | [SEQ ID NO: 1757] | | | GCAUUG |
| | | | | | | | | | [SEQ ID NO: 1954] |
| 3302 | miR 155-M | -2.42428 | 0.285147 | TTGAACAA | CCCAAATC | CCTGGAGGCTTG | UUGAACAA | CCCAAAUC | CCUGGAGGCUU |
| | | | | GGGGCTGA | GCCCTTGTT | CTGAAGGCTGTA | GGGGCUGA | GCCCUUGU | GCUGAAGGCUG |
| | | | | TTTGGG | CAA | TGCTGTTGAACA | UUUGGG | UCAA | UAUGCUGUUGA |
| | | | | [SEQ ID | [SEQ ID | AGGGGCTGATTT | [SEQ ID | [SEQ ID | ACAAGGGGCUG |
| | | | | NO: 688] | NO: 1652] | GGGTTTTGGCCA | NO: 1216] | NO: 1847] | AUUUGGGUUUU |
| | | | | | | CTGACTGACCCA | | | GGCCACUGACU |
| | | | | | | AATCGCCCTTGT | | | GACCCAAAUCG |
| | | | | | | TCAACAGGACA | | | CCCUUGUUCAA |
| | | | | | | CAAGGCCTGTTA | | | CAGGACACAAG |
| | | | | | | CTAGCACTCACA | | | GCCUGUUACUA |
| | | | | | | TGGAACAAATG | | | GCACUCACAUG |
| | | | | | | GCCACCGG | | | GAACAAAUGGC |
| | | | | | | [SEQ ID NO: 1758] | | | CACCGG |
| | | | | | | | | | [SEQ ID NO: 1955] |
| 2586 | miR 155-M | -2.4228 | -0.13209 | TAGATTCAG | CCAAGTTCA | CCTGGAGGCTTG | UAGAUUCA | CCAAGUUC | CCUGGAGGCUU |
| | | | | AAGTAGAA | CTCTGAATC | CTGAAGGCTGTA | GAAGUAGA | ACUCUGAA | GCUGAAGGCUG |
| | | | | CTTGG | TA | TGCTGTAGATTC | ACUUGG | UCUA | UAUGCUGUAGA |
| | | | | [SEQ ID | [SEQ ID | AGAAGTAGAAC | [SEQ ID | [SEQ ID | UUCAGAAGUAG |
| | | | | NO: 1621] | NO: 1653] | TTGGTTTTGGCC | NO: 1816] | NO: 1848] | AACUUGGUUUU |
| | | | | | | ACTGACTGACCA | | | GGCCACUGACU |
| | | | | | | AGTTCACTCTGA | | | GACCAAGUUCA |
| | | | | | | ATCTACAGGAC | | | CUCUGAAUCUA |
| | | | | | | ACAAGGCCTGTT | | | CAGGACACAAG |
| | | | | | | ACTAGCACTCAC | | | GCCUGUUACUA |
| | | | | | | ATGGAACAAAT | | | GCACUCACAUG |
| | | | | | | GGCCACCGG | | | GAACAAAUGGC |
| | | | | | | [SEQ ID NO: 1759] | | | CACCGG |
| | | | | | | | | | [SEQ ID NO: 1956] |
| 1755 | miR-122 | -2.42198 | 0.034447 | TCGGGTTGA | CACACTTCA | GGCTACAGAGTT | UCGGGUUG | CACACUUC | GGCUACAGAGU |
| | | | | AATCTGAA | GACTTCAAC | TCCTTAGCAGAG | AAAUCUGA | AGACUUCA | UUCCUUAGCAG |
| | | | | GTGTG | CATA | CTGTCGGGTTGA | AGUGUG | ACCAUA | AGCUGUCGGGU |
| | | | | [SEQ ID | [SEQ ID | AATCTGAAGTGT | [SEQ ID | [SEQ ID | UGAAAUCUGAA |
| | | | | NO: 657] | NO: 1646] | GTGTCTAAACTA | NO: 1185] | NO: 1841] | GUGUGUGUCUA |
| | | | | | | TCACACTTCAGA | | | AACUAUCACAC |
| | | | | | | CTTCAACCATAT | | | UUCAGACUUCA |
| | | | | | | AGCTACTGCTAG | | | ACCAUAUAGCU |
| | | | | | | GCAATCCTTCCC | | | ACUGCUAGGCA |
| | | | | | | TCGATAAATGTC | | | AUCCUUCCCUC |
| | | | | | | TTGGCATCGTTT | | | GAUAAAUGUCU |
| | | | | | | GCTTTG | | | UGGCAUCGUUU |
| | | | | | | [SEQ ID NO: 1760] | | | GCUUUG |
| | | | | | | | | | [SEQ ID NO: 1957] |
| 2945 | miR-1-1 | -2.42094 | -0.06694 | TGTAGTAG | TGAGCCAA | CATGCAGACTGC | UGUAGUAG | UGAGCCAA | CAUGCAGACUG |
| | | | | AAGGCTTTG | AGCCTTCTA | CTGCTTGGGTGA | AAGGCUUU | AGCCUUCU | CCUGCUUGGGU |
| | | | | GCTGA | CCGACA | GCCAAAGCCTTC | GGCUGA | ACCGACA | GAGCCAAAGCC |
| | | | | [SEQ ID | [SEQ ID | TACCGACATATG | [SEQ ID | [SEQ ID | UUCUACCGACA |
| | | | | NO: 685] | NO: 1633] | GACCTGCTAAGC | NO: 1213] | NO: 1828] | UAUGGACCUGC |
| | | | | | | TATGTAGTAGAA | (Same guide as | | UAAGCUAUGUA |
| | | | | | | GGCTTTGGCTGA | XD-14860) | | GUAGAAGGCUU |
| | | | | | | CTCAGGCCGGG | | | UGGCUGACUCA |
| | | | | | | ACCTCTCTCGCC | | | GGCCGGGACCU |
| | | | | | | GCACTGAGGGG | | | CUCUCGCCGCA |
| | | | | | | CACTCCACACCA | | | CUGAGGGGCAC |
| | | | | | | CGGGGGCC | | | UCCACACCACG |
| | | | | | | [SEQ ID NO: 1761] | | | GGGGCC |
| | | | | | | | | | [SEQ ID NO: 1958 |
| 1755 | miR-132 | -2.41844 | 0.196175 | TCGGGTTGA | CCCACTTCA | GCCGTCCGCGCG | UCGGGUUG | CCCACUUC | GCCGUCCGCGC |
| | | | | AATCTGAA | GAGTGCAA | CCCCGCCCCCGC | AAAUCUGA | AGAGUGCA | GCCCCGCCCCC |
| | | | | GTGTG | CCCGA | GTCTCCAGGGCC | AGUGUG | ACCCGA | GCGUCUCCAGG |
| | | | | TSEQ ID | [SEQ ID | CACTTCAGAGTG | [SEQ ID | [SEQ ID | GCCCACUUCAG |
| | | | | NO: 657] | NO: 1672] | CAACCCGACTGT | NO: 1185] | NO: 1867] | AGUGCAACCCG |
| | | | | | | GGGAACTGGAG | | | ACUGUGGGAAC |
| | | | | | | GTCGGGTTGAA | | | UGGAGGUCGGG |
| | | | | | | ATCTGAAGTGTG | | | UUGAAAUCUGA |
| | | | | | | CCCCGCAGCAC | | | AGUGUGCCCCG |
| | | | | | | GCCCACGCGCC | | | CAGCACGCCCA |
| | | | | | | GCGCCACGCCG | | | CGCGCCGCGCC |
| | | | | | | CGCCCCGAGCC | | | ACGCCGCGCCC |
| | | | | | | [SEQ ID NO: 1762] | | | CGAGCC |
| | | | | | | | | | [SEQ ID NO: 1959] |
| 3301 | miR-1-1 | -2.41082 | 0.051034 | TGAACAAG | TGCCAAATC | CATGCAGACTGC | UGAACAAG | UGCCAAAU | CAUGCAGACUG |
| | | | | GGGCTGATT | AGCCCCTTG | CTGCTTGGGTGC | GGGCUGAU | CAGCCCCU | CCUGCUUGGGU |
| | | | | TGGGA | CGTCA | CAAATCAGCCCC | UUGGGA | UGCGUCA | GCCAAAUCAGC |
| | | | | [SEQ ID | [SEQ ID | TTGCGTCATATG | [SEQ ID | [SEQ ID | CCCUUGCGUCA |
| | | | | NO: 687] | NO: 1673] | GACCTGCTAAGC | NO: 1215] | NO: 1868] | UAUGGACCUGC |
| | | | | | | TATGAACAAGG | | | UAAGCUAUGAA |
| | | | | | | GGCTGATTTGGG | | | CAAGGGGCUGA |
| | | | | | | ACTCAGGCCGG | | | UUUGGGACUCA |
| | | | | | | GACCTCTCTCGC | | | GGCCGGGACCU |
| | | | | | | CGCACTGAGGG | | | CUCUCGCCGCA |
| | | | | | | GCACTCCACACC | | | CUGAGGGGCAC |
| | | | | | | ACGGGGGCC | | | UCCACACCACG |
| | | | | | | [SEQ ID NO: 1763] | | | GGGGCC |
| | | | | | | | | | [SEQ ID NO: 1960] |
| 2602 | miR-132 | -2.40987 | -0.18915 | TTTAGTAGT | ACTCTATGG | GCCGTCCGCGCG | UUUAGUAG | ACUCUAUG | GCCGUCCGCGC |
| | | | | TGATCCATA | ATAACCTAC | CCCCGCCCCCGC | UUGAUCCA | GAUAACCU | GCCCCGCCCCC |
| | | | | GATT | TAAA | GTCTCCAGGGAC | UAGAUU | ACUAAA | GCGUCUCCAGG |
| | | | | SEQ ID | SEQ ID | TCTATGGATAAC | ISEQ ID | [SEQ ID | GACUCUAUGGA |
| | | | | NO: 1616] | NO: 1674] | CTACTAAACTGT | NO: 202] | NO: 1869] | UAACCUACUAA |
| | | | | | | GGGAACTGGAG | (Same guide as | | ACUGUGGGAAC |
| | | | | | | GTTTAGTAGTTG | XD-14837) | | UGGAGGUUUAG |
| | | | | | | ATCCATAGATTC | | | UAGUUGAUCCA |
| | | | | | | CCCGCAGCACG | | | UAGAUUCCCCG |
| | | | | | | CCCACGCGCCGC | | | CAGCACGCCCA |
| | | | | | | GCCACGCCGCG | | | CGCGCCGCGCC |
| | | | | | | CCCCGAGCC | | | ACGCCGCGCCC |
| | | | | | | [SEQ ID NO: 1764] | | | CGAGCC |
| | | | | | | | | | [SEQ ID NO: 1961] |
| 3302 | miR-122 M | -2.40308 | -0.14782 | TTGAACAA | CCCAAATC | GGCTACAGAGTT | UUGAACAA | CCCAAAUC | GGCUACAGAGU |
| | | | | GGGGCTGA | AGCACCTTG | TGCTTAGCAGAG | GGGGCUGA | AGCACCUU | UUGCUUAGCAG |
| | | | | TTTGGG | TTACA | CTGTTGAACAAG | UUUGGG | GUUACA | AGCUGUUGAAC |
| | | | | SEQ ID | [SEQ ID | GGGCTGATTTGG | [SEQ ID | SEQ ID | AAGGGGCUGAU |
| | | | | NO: 688] | NO: 1675] | GTGTCTAAACTA | NO: 1216] | NO: 1870] | UUGGGUGUCUA |
| | | | | | | TCCCAAATCAGC | | | AACUAUCCCAA |
| | | | | | | ACCTTGTTACAT | | | AUCAGCACCUU |
| | | | | | | AGCTACTGCTAG | | | GUUACAUAGCU |
| | | | | | | GCCATCCTTCCC | | | ACUGCUAGGCC |
| | | | | | | TCGATAAATGTC | | | AUCCUUCCCUC |
| | | | | | | TTGGCATCGTTT | | | GAUAAAUGUCU |
| | | | | | | GCTTTG | | | UGGCAUCGUUU |
| | | | | | | [SEQ ID NO: 1765] | | | GCUUUG |
| | | | | | | | | | [SEQ ID NO: 1962] |
| 1755 | miR-155E | -2.39604 | -0.03915 | TCGGGTTGA | CACACTTCG | CTGGAGGCTTGC | UCGGGUUG | CACACUUC | CUGGAGGCUUG |
| | | | | AATCTGAA | ATTCAACCC | TTTGGGCTGTAT | AAAUCUGA | GAUUCAAC | CUUUGGGCUGU |
| | | | | GTGTG | GA | GCTGTCGGGTTG | AGUGUG | CCGA | AUGCUGUCGGG |
| | | | | [SEQ ID | [SEQ ID | AAATCTGAAGT | [SEQ ID | [SEQ ID | UUGAAAUCUGA |
| | | | | NO: 657] | NO: 1676] | GTGTTTTGGCCT | NO: 1185] | NO: 1871] | AGUGUGUUUUG |
| | | | | | | CTGACTGACACA | | | GCCUCUGACUG |
| | | | | | | CTTCGATTCAAC | | | ACACACUUCGA |
| | | | | | | CCGACAGGACA | | | UUCAACCCGAC |
| | | | | | | AGGCCCTTTATC | | | AGGACAAGGCC |
| | | | | | | AGCACTCACATG | | | CUUUAUCAGCA |
| | | | | | | GAACAAATGGC | | | CUCACAUGGAA |
| | | | | | | CACCGTGGG | | | CAAAUGGCCAC |
| | | | | | | [SEQ ID NO: 1766] | | | CGUGGG |
| | | | | | | | | | [SEQ ID NO: 1963] |
| 3842 | miR-130a | -2.39495 | -0.55009 | AACGTGAG | TTCGATCCA | GCAGGGCCGGC | AACGUGAG | UUCGAUCC | GCAGGGCCGGC |
| | | | | AAGGATGG | TACTTCTCA | ATGCCTCTGCTG | AAGGAUGG | AUACUUCU | AUGCCUCUGCU |
| | | | | ATCGTA | TGTC | CTGGCCATTCGA | AUCGUA | CAUGUC | GCUGGCCAUUC |
| | | | | [SEQ ID | [SEQ ID | TCCATACTTCTC | [SEQ ID | [SEQ ID | GAUCCAUACUU |
| | | | | NO: 1625] | NO: 1677] | ATGTCCTGTCTG | NO: 1824] | NO: 1872] | CUCAUGUCCUG |
| | | | | | | CACCTGTCACTA | | | UCUGCACCUGU |
| | | | | | | GAACGTGAGAA | | | CACUAGAACGU |
| | | | | | | GGATGGATCGT | | | GAGAAGGAUGG |
| | | | | | | ATGGCCGTGTAG | | | AUCGUAUGGCC |
| | | | | | | TGCTACCCAGCG | | | GUGUAGUGCUA |
| | | | | | | CTGGCTGCCTCC | | | CCCAGCGCUGG |
| | | | | | | TCAGCATTG | | | CUGCCUCCUCA |
| | | | | | | [SEQ ID NO: 1767] | | | GCAUUG |
| | | | | | | | | | [SEQ ID NO: 1964 |
| 2945 | miR-100 | -2.38747 | -0.10088 | TGTAGTAG | TCAGCCCA | CCCAAAAGAGA | UGUAGUAG | UCAGCCCA | CCCAAAAGAGA |
| | | | | AAGGCTTTG | AGACTTCTA | GAAGATATTGA | AAGGCUUU | AGACUUCU | GAAGAUAUUGA |
| | | | | GCTGA | ATACT | GGCCTGTTGCCA | GGCUGA | AAUACU | GGCCUGUUGCC |
| | | | | SEQ ID | [SEQ ID | CATGTAGTAGA | [SEQ ID | [SEQ ID | ACAUGUAGUAG |
| | | | | NO: 685] | NO: 1678] | AGGCTTTGGCTG | NO: 1213] | NO: 1873] | AAGGCUUUGGC |
| | | | | | | AGTATTAGTCCG | (Same guide as | | UGAGUAUUAGU |
| | | | | | | TCAGCCCAAGA | XD-14860) | | CCGUCAGCCCA |
| | | | | | | CTTCTAATACTT | | | AGACUUCUAAU |
| | | | | | | GTGTCTGTTAGG | | | ACUUGUGUCUG |
| | | | | | | CAATCTCACGGA | | | UUAGGCAAUCU |
| | | | | | | CCTGGGGCTTTG | | | CACGGACCUGG |
| | | | | | | CTTATATGCC | | | GGCUUUGCUUA |
| | | | | | | [SEQ ID NO: 1768] | | | UAUGCC |
| | | | | | | | | | [SEQ ID NO: 1965] |
| 2586 | miR-130a | -2.38521 | 0.042149 | TAGATTCAG | CGAAGTTCT | GCAGGGCCGGC | UAGAUUCA | CGAAGUUC | GCAGGGCCGGC |
| | | | | AAGTAGAA | AATTCTGAA | ATGCCTCTGCTG | GAAGUAGA | UAAUUCUG | AUGCCUCUGCU |
| | | | | CTTGG | TCTC | CTGGCCACGAA | ACUUGG | AAUCUC | GCUGGCCACGA |
| | | | | [SEQ ID | [SEQ ID | GTTCTAATTCTG | [SEQ ID | [SEQ ID | AGUUCUAAUUC |
| | | | | NO: 1621] | NO: 1679] | AATCTCCTGTCT | NO: 1816] | NO: 1874] | UGAAUCUCCUG |
| | | | | | | GCACCTGTCACT | | | UCUGCACCUGU |
| | | | | | | AGTAGATTCAG | | | CACUAGUAGAU |
| | | | | | | AAGTAGAACTT | | | UCAGAAGUAGA |
| | | | | | | GGTGGCCGTGTA | | | ACUUGGUGGCC |
| | | | | | | GTGCTACCCAGC | | | GUGUAGUGCUA |
| | | | | | | GCTGGCTGCCTC | | | CCCAGCGCUGG |
| | | | | | | CTCAGCATTG | | | CUGCCUCCUCA |
| | | | | | | [SEQ ID NO: 1769] | | | GCAUUG |
| | | | | | | | | | [SEQ ID NO: 1966] |
| 3301 | miR-1-1_M | -2.38494 | 0.327128 | TGAACAAG | TGCCAAATC | CATGCAGACTGC | UGAACAAG | UGCCAAAU | CAUGCAGACUG |
| | | | | GGGCTGATT | AGCCCCTTG | CTGCTTGGGTGC | GGGCUGAU | CAGCCCCU | CCUGCUUGGGU |
| | | | | TGGGA | CGTCA | CAAATCAGCCCC | UUGGGA | UGCGUCA | GCCAAAUCAGC |
| | | | | [SEQ ID | [SEQ ID | TTGCGTCATATG | [SEQ ID | [SEQ ID | CCCUUGCGUCA |
| | | | | NO: 687] | NO: 1673] | GACCTGCTAAGC | NO: 1215] | NO: 1868] | UAUGGACCUGC |
| | | | | | | TATGAACAAGG | | | UAAGCUAUGAA |
| | | | | | | GGCTGATTTGGG | | | CAAGGGGCUGA |
| | | | | | | ACTCAGGCCGG | | | UUUGGGACUCA |
| | | | | | | GACCTCTTCCGC | | | GGCCGGGACCU |
| | | | | | | CGCACTGAGGG | | | CUUCCGCCGCA |
| | | | | | | GCACTCCACACC | | | CUGAGGGGCAC |
| | | | | | | ACGGGGGCC | | | UCCACACCACG |
| | | | | | | [SEQ ID NO: 1770] | | | GGGGCC |
| | | | | | | | | | [SEQ ID NO: 1967] |
| 1755 | miR-190a | -2.38113 | 0.329455 | TCGGGTTGA | CACTTCAGA | GAGCTCAGTCA | UCGGGUUG | CACUUCAG | GAGCUCAGUCA |
| | | | | AATCTGAA | TTTCAACCC | AACCTGGATGCC | AAAUCUGA | AUUUCAAC | AACCUGGAUGC |
| | | | | GTGTG | AC | TTTTCTGCAGGC | AGUGUG | CCAC | CUUUUCUGCAG |
| | | | | [SEQ ID | [SEQ ID | CTCTGTGTCGGG | [SEQ ID | [SEQ ID | GCCUCUGUGUC |
| | | | | NO: 657] | NO: 1680] | TTGAAATCTGAA | NO: 1185] | NO: 1875] | GGGUUGAAAUC |
| | | | | | | GTGTGTGTTATT | | | UGAAGUGUGUG |
| | | | | | | TAATCCACACTT | | | UUAUUUAAUCC |
| | | | | | | CAGATTTCAACC | | | ACACUUCAGAU |
| | | | | | | CACCTACAGTGT | | | UUCAACCCACC |
| | | | | | | CTTGCCCTGTCT | | | UACAGUGUCUU |
| | | | | | | CCGGGGGTTCCT | | | GCCCUGUCUCC |
| | | | | | | AATAAAG | | | GGGGGUUCCUA |
| | | | | | | [SEQ ID NO: 1771] | | | AUAAAG |
| | | | | | | | | | [SEQ ID NO: 1968] |
| 2602 | miR-1-1_M | -2.37919 | 0.040602 | TTTAGTAGT | ATTCTATGG | CATGCAGACTGC | UUUAGUAG | AUUCUAUG | CAUGCAGACUG |
| | | | | TGATCCATA | ATCAACTAC | CTGCTTGGGATT | UUGAUCCA | GAUCAACU | CCUGCUUGGGA |
| | | | | GATT | CGAAA | CTATGGATCAAC | UAGAUU | ACCGAAA | UUCUAUGGAUC |
| | | | | [SEQ ID | [SEQ ID | TACCGAAATATG | [SEQ ID | [SEQ ID | AACUACCGAAA |
| | | | | NO: 1616] | NO: 1681] | GACCTGCTAAGC | NO: 202] | NO: 1876] | UAUGGACCUGC |
| | | | | | | TATTTAGTAGTT | (Same guide as | | UAAGCUAUUUA |
| | | | | | | GATCCATAGATT | XD-14837) | | GUAGUUGAUCC |
| | | | | | | CTCAGGCCGGG | | | AUAGAUUCUCA |
| | | | | | | ACCTCTTCCGCC | | | GGCCGGGACCU |
| | | | | | | GCACTGAGGGG | | | CUUCCGCCGCA |
| | | | | | | CACTCCACACCA | | | CUGAGGGGCAC |
| | | | | | | CGGGGGCC | | | UCCACACCACG |
| | | | | | | [SEQ ID NO: 1772] | | | GGGGCC |
| | | | | | | | | | [SEQ ID NO: 1969] |
| 1231 | miR-100_M | -2.37228 | -0.26919 | TTCACTTTA | CTGCTACCA | CCCAAAAGAGA | UUCACUUU | CUGCUACC | CCCAAAAGAGA |
| | | | | GCACTGAT | GGGCTAAA | GAAGATATTGAT | AGCACUGA | AGGGCUAA | GAAGAUAUUGA |
| | | | | AGCAG | ATGAT | GCCTGTTGCCAC | UAGCAG | AAUGAU | UGCCUGUUGCC |
| | | | | [SEQ ID | [SEQ ID | ATTCACTTTAGC | [SEQ ID | [SEQ ID | ACAUUCACUUU |
| | | | | NO: 1627] | NO: 1682] | ACTGATAGCAG | NO: 1825] | NO: 1877] | AGCACUGAUAG |
| | | | | | | GTATTAGTCCGC | | | CAGGUAUUAGU |
| | | | | | | TGCTACCAGGGC | | | CCGCUGCUACC |
| | | | | | | TAAAATGATTGT | | | AGGGCUAAAAU |
| | | | | | | GTCTGTTAGGCT | | | GAUUGUGUCUG |
| | | | | | | ATTCCACGGACC | | | UUAGGCUAUUC |
| | | | | | | TGGGGCTTTGCT | | | CACGGACCUGG |
| | | | | | | TATATGCC | | | GGCUUUGCUUA |
| | | | | | | [SEQ ID NO: 1773] | | | UAUGCC |
| | | | | | | | | | [SEQ ID NO: 1970] |
| 3273 | miR-155E | -2.36984 | -0.0801 | TAGGACTGT | CAATATGTG | CTGGAGGCTTGC | UAGGACUG | CAAUAUGU | CUGGAGGCUUG |
| | | | | AGGCAACA | CCACAGTCC | TTTGGGCTGTAT | UAGGCAAC | GCCACAGU | CUUUGGGCUGU |
| | | | | TATTG | TA | GCTGTAGGACTG | AUAUUG | CCUA | AUGCUGUAGGA |
| | | | | [SEQ ID | [SEQ ID | TAGGCAACATAT | [SEQ ID | [SEQ ID | CUGUAGGCAAC |
| | | | | NO: 1628] | NO: 1683] | TGTTTTGGCCTC | NO: 1821] | NO: 1878] | AUAUUGUUUUG |
| | | | | | | TGACTGACAATA | | | GCCUCUGACUG |
| | | | | | | TGTGCCACAGTC | | | ACAAUAUGUGC |
| | | | | | | CTACAGGACAA | | | CACAGUCCUAC |
| | | | | | | GGCCCTTTATCA | | | AGGACAAGGCC |
| | | | | | | GCACTCACATGG | | | CUUUAUCAGCA |
| | | | | | | AACAAATGGCC | | | CUCACAUGGAA |
| | | | | | | ACCGTGGG | | | CAAAUGGCCAC |
| | | | | | | [SEQ ID NO: 1774] | | | CGUGGG |
| | | | | | | | | | [SEQ ID NO: 1971] |
| 3301 | miR-190a | -2.36785 | 0.195332 | TGAACAAG | TCCAATCAG | GAGCTCAGTCA | UGAACAAG | UCCAAUCA | GAGCUCAGUCA |
| | | | | GGGCTGATT | CCCCTTGTT | AACCTGGATGCC | GGGCUGAU | GCCCCUUG | AACCUGGAUGC |
| | | | | TGGGA | AC | TTTTCTGCAGGC | UUGGGA | UUAC | CUUUUCUGCAG |
| | | | | [SEQ ID | [SEQ ID | CTCTGTGTGAAC | [SEQ ID | [SEQ ID | GCCUCUGUGUG |
| | | | | NO: 687] | NO: 1684] | AAGGGGCTGAT | NO: 1215] | NO: 1879] | AACAAGGGGCU |
| | | | | | | TTGGGATGTTAT | | | GAUUUGGGAUG |
| | | | | | | TTAATCCATCCA | | | UUAUUUAAUCC |
| | | | | | | ATCAGCCCCTTG | | | AUCCAAUCAGC |
| | | | | | | TTACCTACAGTG | | | CCCUUGUUACC |
| | | | | | | TCTTGCCCTGTC | | | UACAGUGUCUU |
| | | | | | | TCCGGGGGTTCC | | | GCCCUGUCUCC |
| | | | | | | TAATAAAG | | | GGGGGUUCCUA |
| | | | | | | [SEQ ID NO: 1775] | | | AUAAAG |
| | | | | | | | | | [SEQ ID NO: 1972] |
| 3043 | miR-100_M | -2.35911 | 0.045827 | TTTGGTGCA | AGCCTGCTT | CCCAAAAGAGA | UUUGGUGC | AGCCUGCU | CCCAAAAGAGA |
| | | | | AAACAAAC | GGTTTGCAA | GAAGATATTGAT | AAAACAAA | UGGUUUGC | GAAGAUAUUGA |
| | | | | AGGCT | CAAT | GCCTGTTGCCAC | CAGGCU | AACAAU | UGCCUGUUGCC |
| | | | | [SEQ ID | [SEQ ID | ATTTGGTGCAAA | [SEQ ID | [SEQ ID | ACAUUUGGUGC |
| | | | | NO: 1615] | NO: 1639] | ACAAACAGGCT | NO: 1812] | NO: 1834] | AAAACAAACAG |
| | | | | | | GTATTAGTCCGA | | | GCUGUAUUAGU |
| | | | | | | GCCTGCTTGGTT | | | CCGAGCCUGCU |
| | | | | | | TGCAACAATTGT | | | UGGUUUGCAAC |
| | | | | | | GTCTGTTAGGCT | | | AAUUGUGUCUG |
| | | | | | | ATTCCACGGACC | | | UUAGGCUAUUC |
| | | | | | | TGGGGCTTTGCT | | | CACGGACCUGG |
| | | | | | | TATATGCC | | | GGCUUUGCUUA |
| | | | | | | [SEQ ID NO: 1776] | | | UAUGCC |
| | | | | | | | | | [SEQ ID NO: 1973] |
| 2586 | miR-122 | -2.35707 | 0.153236 | TAGATTCAG | CCAAGTTCT | GGCTACAGAGTT | UAGAUUCA | CCAAGUUC | GGCUACAGAGU |
| | | | | AAGTAGAA | ACCTCTGAA | TCCTTAGCAGAG | GAAGUAGA | UACCUCUG | UUCCUUAGCAG |
| | | | | CTTGG | TAGA | CTGTAGATTCAG | ACUUGG | AAUAGA | AGCUGUAGAUU |
| | | | | [SEQ ID | [SEQ ID | AAGTAGAACTT | [SEQ ID | [SEQ ID | CAGAAGUAGAA |
| | | | | NO: 1621] | NO: 1685] | GGTGTCTAAACT | NO: 1816] | NO: 1880] | CUUGGUGUCUA |
| | | | | | | ATCCAAGTTCTA | | | AACUAUCCAAG |
| | | | | | | CCTCTGAATAGA | | | UUCUACCUCUG |
| | | | | | | TAGCTACTGCTA | | | AAUAGAUAGCU |
| | | | | | | GGCAATCCTTCC | | | ACUGCUAGGCA |
| | | | | | | CTCGATAAATGT | | | AUCCUUCCCUC |
| | | | | | | CTTGGCATCGTT | | | GAUAAAUGUCU |
| | | | | | | TGCTTTG | | | UGGCAUCGUUU |
| | | | | | | [SEQ ID NO: 1777] | | | GCUUUG |
| | | | | | | | | | [SEQ ID NO: 1974] |
| 1755 | miR- | -2.34216 | 0.272299 | TCGGGTTGA | CACTTCAGA | GAGCTCAGTCA | UCGGGUUG | CACUUCAG | GAGCUCAGUCA |
| | 190a_M | | | AATCTGAA | TTTCAACCC | AACCTGGATGCC | AAAUCUGA | AUUUCAAC | AACCUGGAUGC |
| | | | | GTGTG | AC | TTTTCTGCAGGC | AGUGUG | CCAC | CUUUUCUGCAG |
| | | | | [SEQ ID | [SEQ ID | GTCTGTGTCGGG | [SEQ ID | [SEQ ID | GCGUCUGUGUC |
| | | | | NO: 657] | NO: 1680] | TTGAAATCTGAA | NO: 1185] | NO: 1875] | GGGUUGAAAUC |
| | | | | | | GTGTGTGTTATT | | | UGAAGUGUGUG |
| | | | | | | TAATCCACACTT | | | UUAUUUAAUCC |
| | | | | | | CAGATTTCAACC | | | ACACUUCAGAU |
| | | | | | | CACCTACAGTCT | | | UUCAACCCACC |
| | | | | | | CTTGCCCTGTCT | | | UACAGUCUCUU |
| | | | | | | CCGGGGGTTCCT | | | GCCCUGUCUCC |
| | | | | | | AATAAAG | | | GGGGGUUCCUA |
| | | | | | | [SEQ ID NO: 1778] | | | AUAAAG |
| | | | | | | | | | [SEQ ID NO: 1975] |
| 1784 | miR-132 | -2.34143 | -0.26117 | ATTAACTAC | TCCAGACC | GCCGTCCGCGCG | AUUAACUA | UCCAGACC | GCCGUCCGCGC |
| | | | | TCTTTGGTC | AAATATTA | CCCCGCCCCCGC | CUCUUUGG | AAAUAUUA | GCCCCGCCCCC |
| | | | | TGAA | GTTAAT | GTCTCCAGGGTC | UCUGAA | GUUAAU | GCGUCUCCAGG |
| | | | | [SEQ ID | [SEQ ID | CAGACCAAATA | [SEQ ID | [SEQ ID | GUCCAGACCAA |
| | | | | NO: 608] | NO: 1686] | TTAGTTAATCTG | NO: 112] | NO: 1881] | AUAUUAGUUAA |
| | | | | | | TGGGAACTGGA | (Same guide as | | UCUGUGGGAAC |
| | | | | | | GGATTAACTACT | XD-14792) | | UGGAGGAUUAA |
| | | | | | | CTTTGGTCTGAA | | | CUACUCUUUGG |
| | | | | | | CCCCGCAGCAC | | | UCUGAACCCCG |
| | | | | | | GCCCACGCGCC | | | CAGCACGCCCA |
| | | | | | | GCGCCACGCCG | | | CGCGCCGCGCC |
| | | | | | | CGCCCCGAGCC | | | ACGCCGCGCCC |
| | | | | | | [SEQ ID NO: 1779] | | | CGAGCC |
| | | | | | | | | | [SEQ ID NO: 1976] |
| 3272 | miR-155E | -2.34055 | -0.19485 | AGGACTGT | GCAATATGT | CTGGAGGCTTGC | AGGACUGU | GCAAUAUG | CUGGAGGCUUG |
| | | | | AGGCAACA | GCTACAGTC | TTTGGGCTGTAT | AGGCAACA | UGCUACAG | CUUUGGGCUGU |
| | | | | TATTGC | CT | GCTGAGGACTGT | UAUUGC | UCCU | AUGCUGAGGAC |
| | | | | [SEQ ID | [SEQ ID | AGGCAACATATT | [SEQ ID | [SEQ ID | UGUAGGCAACA |
| | | | | NO: 1618] | NO: 1687] | GCTTTTGGCCTC | NO: 1814] | NO: 1882] | UAUUGCUUUUG |
| | | | | | | TGACTGAGCAAT | | | GCCUCUGACUG |
| | | | | | | ATGTGCTACAGT | | | AGCAAUAUGUG |
| | | | | | | CCTCAGGACAA | | | CUACAGUCCUC |
| | | | | | | GGCCCTTTATCA | | | AGGACAAGGCC |
| | | | | | | GCACTCACATGG | | | CUUUAUCAGCA |
| | | | | | | AACAAATGGCC | | | CUCACAUGGAA |
| | | | | | | ACCGTGGG | | | CAAAUGGCCAC |
| | | | | | | [SEQ ID NO: 1780] | | | CGUGGG |
| | | | | | | | | | [SEQ ID NO: 1977] |
| 1159 | miR-1-1_M | -2.33035 | 0.359288 | TGTACCACA | GATCAGAC | CATGCAGACTGC | UGUACCAC | GAUCAGAC | CAUGCAGACUG |
| | | | | ACAAAGTC | TTTGTTGTG | CTGCTTGGGGAT | AACAAAGU | UUUGUUGU | CCUGCUUGGGG |
| | | | | TGAAC | GCGACA | CAGACTTTGTTG | CUGAAC | GGCGACA | AUCAGACUUUG |
| | | | | [SEQ ID | [SEQ ID | TGGCGACATATG | [SEQ ID | [SEQ ID | UUGUGGCGACA |
| | | | | NO: 603] | NO: 1688] | GACCTGCTAAGC | NO: 40] | NO: 1883] | UAUGGACCUGC |
| | | | | | | TATGTACCACAA | (Same guide as | | UAAGCUAUGUA |
| | | | | | | CAAAGTCTGAA | XD-14756) | | CCACAACAAAG |
| | | | | | | CCTCAGGCCGG | | | UCUGAACCUCA |
| | | | | | | GACCTCTTCCGC | | | GGCCGGGACCU |
| | | | | | | CGCACTGAGGG | | | CUUCCGCCGCA |
| | | | | | | GCACTCCACACC | | | CUGAGGGGCAC |
| | | | | | | ACGGGGGCC | | | UCCACACCACG |
| | | | | | | [SEQ ID NO: 1781] | | | GGGGCC |
| | | | | | | | | | [SEQ ID NO: 1978] |
| 3269 | miR- | -2.3297 | 0.037293 | ACTGTAGG | CACAATAT | GAGCTCAGTCA | ACUGUAGG | CACAAUAU | GAGCUCAGUCA |
| | 190a_M | | | CAACATATT | GTTGCCTAC | AACCTGGATGCC | CAACAUAU | GUUGCCUA | AACCUGGAUGC |
| | | | | GCGTG | AAG | TTTTCTGCAGGC | UGCGUG | CAAG | CUUUUCUGCAG |
| | | | | SEQ ID | [SEQ ID | GTCTGTGACTGT | [SEQ ID | [SEQ ID | GCGUCUGUGAC |
| | | | | NO: 1629] | NO: 1689] | AGGCAACATATT | NO: 1822] | NO: 1884] | UGUAGGCAACA |
| | | | | | | GCGTGTGTTATT | | | UAUUGCGUGUG |
| | | | | | | TAATCCACACAA | | | UUAUUUAAUCC |
| | | | | | | TATGTTGCCTAC | | | ACACAAUAUGU |
| | | | | | | AAGCTACAGTCT | | | UGCCUACAAGC |
| | | | | | | CTTGCCCTGTCT | | | UACAGUCUCUU |
| | | | | | | CCGGGGGTTCCT | | | GCCCUGUCUCC |
| | | | | | | AATAAAG | | | GGGGGUUCCUA |
| | | | | | | [SEQ ID NO: 1782] | | | AUAAAG |
| | | | | | | | | | [SEQ ID NO: 1979] |
| 3043 | miR-1-1 | -2.32683 | 0.097086 | TTTGGTGCA | ACCCTGTTT | CATGCAGACTGC | UUUGGUGC | ACCCUGUU | CAUGCAGACUG |
| | | | | AAACAAAC | GTTTTGCAC | CTGCTTGGGACC | AAAACAAA | UGUUUUGC | CCUGCUUGGGA |
| | | | | AGGCT | GAAAA | CTGTTTGTTTTG | CAGGCU | ACGAAAA | CCCUGUUUGUU |
| | | | | [SEQ ID | [SEQ ID | CACGAAAATAT | [SEQ ID | [SEQ ID | UUGCACGAAAA |
| | | | | NO: 1615] | NO: 1690] | GGACCTGCTAA | NO: 1812] | NO: 1885] | UAUGGACCUGC |
| | | | | | | GCTATTTGGTGC | | | UAAGCUAUUUG |
| | | | | | | AAAACAAACAG | | | GUGCAAAACAA |
| | | | | | | GCTCTCAGGCCG | | | ACAGGCUCUCA |
| | | | | | | GGACCTCTCTCG | | | GGCCGGGACCU |
| | | | | | | CCGCACTGAGG | | | CUCUCGCCGCA |
| | | | | | | GGCACTCCACAC | | | CUGAGGGGCAC |
| | | | | | | CACGGGGGCC | | | UCCACACCACG |
| | | | | | | [SEQ ID NO: 1783] | | | GGGGCC |
| | | | | | | | | | [SEQ ID NO: 1980] |
| 3273 | miR-1-1 | -2.32464 | -0.13929 | TAGGACTGT | CTATATGTT | CATGCAGACTGC | UAGGACUG | CUAUAUGU | CAUGCAGACUG |
| | | | | AGGCAACA | GCCTACAGT | CTGCTTGGGCTA | UAGGCAAC | UGCCUACA | CCUGCUUGGGC |
| | | | | TATTG | GACTA | TATGTTGCCTAC | AUAUUG | GUGACUA | UAUAUGUUGCC |
| | | | | [SEQ ID | [SEQ ID | AGTGACTATATG | [SEQ ID | [SEQ ID | UACAGUGACUA |
| | | | | NO: 1628] | NO: 1691] | GACCTGCTAAGC | NO: 1821] | NO: 1886] | UAUGGACCUGC |
| | | | | | | TATAGGACTGTA | | | UAAGCUAUAGG |
| | | | | | | GGCAACATATTG | | | ACUGUAGGCAA |
| | | | | | | CTCAGGCCGGG | | | CAUAUUGCUCA |
| | | | | | | ACCTCTCTCGCC | | | GGCCGGGACCU |
| | | | | | | GCACTGAGGGG | | | CUCUCGCCGCA |
| | | | | | | CACTCCACACCA | | | CUGAGGGGCAC |
| | | | | | | CGGGGGCC | | | UCCACACCACG |
| | | | | | | [SEQ ID NO: 1784] | | | GGGGCC |
| | | | | | | | | | [SEQ ID NO: 1981] |
| 2586 | miR-100 | -2.32164 | -0.18294 | TAGATTCAG | CCAAGTCCT | CCCAAAAGAGA | UAGAUUCA | CCAAGUCC | CCCAAAAGAGA |
| | | | | AAGTAGAA | AATTCTGAC | GAAGATATTGA | GAAGUAGA | UAAUUCUG | GAAGAUAUUGA |
| | | | | CTTGG | TCTT | GGCCTGTTGCCA | ACUUGG | ACUCUU | GGCCUGUUGCC |
| | | | | [SEQ ID | [SEQ ID | CATAGATTCAGA | [SEQ ID | [SEQ ID | ACAUAGAUUCA |
| | | | | NO: 1621] | NO: 1692] | AGTAGAACTTG | NO: 1816] | NO: 1887] | GAAGUAGAACU |
| | | | | | | GGTATTAGTCCG | | | UGGGUAUUAGU |
| | | | | | | CCAAGTCCTAAT | | | CCGCCAAGUCC |
| | | | | | | TCTGACTCTTTG | | | UAAUUCUGACU |
| | | | | | | TGTCTGTTAGGC | | | CUUUGUGUCUG |
| | | | | | | AATCTCACGGAC | | | UUAGGCAAUCU |
| | | | | | | CTGGGGCTTTGC | | | CACGGACCUGG |
| | | | | | | TTATATGCC | | | GGCUUUGCUUA |
| | | | | | | [SEQ ID NO: 1785] | | | UAUGCC |
| | | | | | | | | | [SEQ ID NO: 1982] |
| 2585 | miR-1-1_M | -2.31178 | 0.12336 | AGATTCAG | GGCAAGTT | CATGCAGACTGC | AGAUUCAG | GGCAAGUU | CAUGCAGACUG |
| | | | | AAGTAGAA | CTACTTCTG | CTGCTTGGGGGC | AAGUAGAA | CUACUUCU | CCUGCUUGGGG |
| | | | | CTTGGC | ACCTCT | AAGTTCTACTTC | CUUGGC | GACCUCU | GCAAGUUCUAC |
| | | | | [SEQ ID | [SEQ ID | TGACCTCTTATG | [SEQ ID | [SEQ ID | UUCUGACCUCU |
| | | | | NO: 1630] | NO: 1693] | GACCTGCTAAGC | NO: 1823] | NO: 1888] | UAUGGACCUGC |
| | | | | | | TAAGATTCAGA | | | UAAGCUAAGAU |
| | | | | | | AGTAGAACTTG | | | UCAGAAGUAGA |
| | | | | | | GCCTCAGGCCG | | | ACUUGGCCUCA |
| | | | | | | GGACCTCTTCCG | | | GGCCGGGACCU |
| | | | | | | CCGCACTGAGG | | | CUUCCGCCGCA |
| | | | | | | GGCACTCCACAC | | | CUGAGGGGCAC |
| | | | | | | CACGGGGGCC | | | UCCACACCACG |
| | | | | | | [SEQ ID NO: 1786] | | | GGGGCC |
| | | | | | | | | | [SEQ ID NO: 1983] |
| 3273 | miR-122 | -2.30907 | -0.11515 | TAGGACTGT | CAATATGTT | GGCTACAGAGTT | UAGGACUG | CAAUAUGU | GGCUACAGAGU |
| | | | | AGGCAACA | GCATACAG | TCCTTAGCAGAG | UAGGCAAC | UGCAUACA | UUCCUUAGCAG |
| | | | | TATTG | TCAGA | CTGTAGGACTGT | AUAUUG | GUCAGA | AGCUGUAGGAC |
| | | | | [SEQ ID | [SEQ ID | AGGCAACATATT | [SEQ ID | [SEQ ID | UGUAGGCAACA |
| | | | | NO: 1628] | NO: 1694] | GTGTCTAAACTA | NO: 1821] | NO: 1889] | UAUUGUGUCUA |
| | | | | | | TCAATATGTTGC | | | AACUAUCAAUA |
| | | | | | | ATACAGTCAGAT | | | UGUUGCAUACA |
| | | | | | | AGCTACTGCTAG | | | GUCAGAUAGCU |
| | | | | | | GCAATCCTTCCC | | | ACUGCUAGGCA |
| | | | | | | TCGATAAATGTC | | | AUCCUUCCCUC |
| | | | | | | TTGGCATCGTTT | | | GAUAAAUGUCU |
| | | | | | | GCTTTG | | | UGGCAUCGUUU |
| | | | | | | [SEQ ID NO: 1787] | | | GCUUUG |
| | | | | | | | | | [SEQ ID NO: 1984] |
| 3301 | miR-100_M | -2.30882 | -0.2279 | TGAACAAG | TCCCAACTC | CCCAAAAGAGA | UGAACAAG | UCCCAACU | CCCAAAAGAGA |
| | | | | GGGCTGATT | ATCCCCTTA | GAAGATATTGAT | GGGCUGAU | CAUCCCCU | GAAGAUAUUGA |
| | | | | TGGGA | TTCT | GCCTGTTGCCAC | UUGGGA | UAUUCU | UGCCUGUUGCC |
| | | | | [SEQ ID | [SEQ ID | ATGAACAAGGG | [SEQ ID | [SEQ ID | ACAUGAACAAG |
| | | | | NO: 687] | NO: 1695] | GCTGATTTGGGA | NO: 1215] | NO: 1890] | GGGCUGAUUUG |
| | | | | | | GTATTAGTCCGT | | | GGAGUAUUAGU |
| | | | | | | CCCAACTCATCC | | | CCGUCCCAACU |
| | | | | | | CCTTATTCTTGT | | | CAUCCCCUUAU |
| | | | | | | GTCTGTTAGGCT | | | UCUUGUGUCUG |
| | | | | | | ATTCCACGGACC | | | UUAGGCUAUUC |
| | | | | | | TGGGGCTTTGCT | | | CACGGACCUGG |
| | | | | | | TATATGCC | | | GGCUUUGCUUA |
| | | | | | | [SEQ ID NO: 1788] | | | UAUGCC |
| | | | | | | | | | [SEQ ID NO: 1985] |
| 2602 | miR-130a | -2.30701 | -0.14983 | TTTAGTAGT | ATTCTATGG | GCAGGGCCGGC | UUUAGUAG | AUUCUAUG | GCAGGGCCGGC |
| | | | | TGATCCATA | AGCAACTA | ATGCCTCTGCTG | UUGAUCCA | GAGCAACU | AUGCCUCUGCU |
| | | | | GATT | TTAAC | CTGGCCAATTCT | UAGAUU | AUUAAC | GCUGGCCAAUU |
| | | | | [SEQ ID | [SEQ ID | ATGGAGCAACT | [SEQ ID | [SEQ ID | CUAUGGAGCAA |
| | | | | NO: 1616] | NO: 1696] | ATTAACCTGTCT | NO: 202] | NO: 1891] | CUAUUAACCUG |
| | | | | | | GCACCTGTCACT | (Same guide as | | UCUGCACCUGU |
| | | | | | | AGTTTAGTAGTT | XD-14837) | | CACUAGUUUAG |
| | | | | | | GATCCATAGATT | | | UAGUUGAUCCA |
| | | | | | | TGGCCGTGTAGT | | | UAGAUUUGGCC |
| | | | | | | GCTACCCAGCGC | | | GUGUAGUGCUA |
| | | | | | | TGGCTGCCTCCT | | | CCCAGCGCUGG |
| | | | | | | CAGCATTG | | | CUGCCUCCUCA |
| | | | | | | [SEQ ID NO: 1789] | | | GCAUUG |
| | | | | | | | | | SEQ ID NO: 1986] |
| 3255 | miR- | -2.30348 | 0.418205 | ATTGCGTGG | CCAAGCTTA | GAGCTCAGTCA | AUUGCGUG | CCAAGCUU | GAGCUCAGUCA |
| | 190a_M | | | AGTAAGCT | CTCCACGCA | AACCTGGATGCC | GAGUAAGC | ACUCCACG | AACCUGGAUGC |
| | | | | GGTGG | CG | TTTTCTGCAGGC | UGGUGG | CACG | CUUUUCUGCAG |
| | | | | [SEQ ID | [SEQ ID | GTCTGTGATTGC | [SEQ ID | [SEQ ID | GCGUCUGUGAU |
| | | | | NO: 617] | NO: 1697] | GTGGAGTAAGC | NO: 306] | NO: 1892] | UGCGUGGAGUA |
| | | | | | | TGGTGGTGTTAT | (Same guide as | | AGCUGGUGGUG |
| | | | | | | TTAATCCACCAA | XD-14889) | | UUAUUUAAUCC |
| | | | | | | GCTTACTCCACG | | | ACCAAGCUUAC |
| | | | | | | CACGCTACAGTC | | | UCCACGCACGC |
| | | | | | | TCTTGCCCTGTC | | | UACAGUCUCUU |
| | | | | | | TCCGGGGGTTCC | | | GCCCUGUCUCC |
| | | | | | | TAATAAAG | | | GGGGGUUCCUA |
| | | | | | | [SEQ ID NO: 1790] | | | AUAAAG |
| | | | | | | | | | [SEQ ID NO: 1987] |
| 3338 | miR-132 | -2.2984 | -0.03531 | TACATGAG | TAAGTCTCA | GCCGTCCGCGCG | UACAUGAG | UAAGUCUC | GCCGUCCGCGC |
| | | | | GATGCTGA | GCCTACTCA | CCCCGCCCCCGC | GAUGCUGA | AGCCUACU | GCCCCGCCCCC |
| | | | | GACTGA | TGTA | GTCTCCAGGGTA | GACUGA | CAUGUA | GCGUCUCCAGG |
| | | | | [SEQ ID | [SEQ ID | AGTCTCAGCCTA | [SEQ ID | [SEQ ID | GUAAGUCUCAG |
| | | | | NO: 1620] | NO: 1698] | CTCATGTACTGT | NO: 314] | NO: 1893] | CCUACUCAUGU |
| | | | | | | GGGAACTGGAG | (Same guide as | | ACUGUGGGAAC |
| | | | | | | GTACATGAGGA | XD-14893) | | UGGAGGUACAU |
| | | | | | | TGCTGAGACTGA | | | GAGGAUGCUGA |
| | | | | | | CCCCGCAGCAC | | | GACUGACCCCG |
| | | | | | | GCCCACGCGCC | | | CAGCACGCCCA |
| | | | | | | GCGCCACGCCG | | | CGCGCCGCGCC |
| | | | | | | CGCCCCGAGCC | | | ACGCCGCGCCC |
| | | | | | | [SEQ ID NO: 1791] | | | CGAGCC |
| | | | | | | | | | [SEQ ID NO: 1988] |
| 3842 | miR-1-1 | -2.27963 | -0.68707 | AACGTGAG | TTCGATCCA | CATGCAGACTGC | AACGUGAG | UUCGAUCC | CAUGCAGACUG |
| | | | | AAGGATGG | TCCTTCTCA | CTGCTTGGGTTC | AAGGAUGG | AUCCUUCU | CCUGCUUGGGU |
| | | | | ATCGTA | GAGTT | GATCCATCCTTC | AUCGUA | CAGAGUU | UCGAUCCAUCC |
| | | | | [SEQ ID | [SEQ ID | TCAGAGTTTATG | [SEQ ID | [SEQ ID | UUCUCAGAGUU |
| | | | | NO: 1625] | NO: 1699] | GACCTGCTAAGC | NO: 1824] | NO: 1894] | UAUGGACCUGC |
| | | | | | | TAAACGTGAGA | | | UAAGCUAAACG |
| | | | | | | AGGATGGATCG | | | UGAGAAGGAUG |
| | | | | | | TACTCAGGCCGG | | | GAUCGUACUCA |
| | | | | | | GACCTCTCTCGC | | | GGCCGGGACCU |
| | | | | | | CGCACTGAGGG | | | CUCUCGCCGCA |
| | | | | | | GCACTCCACACC | | | CUGAGGGGCAC |
| | | | | | | ACGGGGGCC | | | UCCACACCACG |
| | | | | | | [SEQ ID NO: 1792] | | | GGGGCC |
| | | | | | | | | | [SEQ ID NO: 1989] |
| 3330 | miR-155E | -2.26554 | 0.033188 | TATGCTGAG | CCACATTAC | CTGGAGGCTTGC | UAUGCUGA | CCACAUUA | CUGGAGGCUUG |
| | | | | ACTGATAAT | AGCTCAGC | TTTGGGCTGTAT | GACUGAUA | CAGCUCAG | CUUUGGGCUGU |
| | | | | GTGG | ATA | GCTGTATGCTGA | AUGUGG | CAUA | AUGCUGUAUGC |
| | | | | SEQ ID | [SEQ ID | GACTGATAATGT | [SEQ ID | SEQ ID | UGAGACUGAUA |
| | | | | NO: 1614] | NO: 1700] | GGTTTTGGCCTC | NO: 1811] | NO: 1895] | AUGUGGUUUUG |
| | | | | | | TGACTGACCACA | | | GCCUCUGACUG |
| | | | | | | TTACAGCTCAGC | | | ACCACAUUACA |
| | | | | | | ATACAGGACAA | | | GCUCAGCAUAC |
| | | | | | | GGCCCTTTATCA | | | AGGACAAGGCC |
| | | | | | | GCACTCACATGG | | | CUUUAUCAGCA |
| | | | | | | AACAAATGGCC | | | CUCACAUGGAA |
| | | | | | | ACCGTGGG | | | CAAAUGGCCAC |
| | | | | | | [SEQ ID NO: 1793] | | | CGUGGG |
| | | | | | | | | | [SEQ ID NO: 1990] |
| 1162 | miR-122 | -2.2647 | -0.33024 | AACTGTACC | CAGACTTTG | GGCTACAGAGTT | AACUGUAC | CAGACUUU | GGCUACAGAGU |
| | | | | ACAACAAA | TTATGGTAC | TCCTTAGCAGAG | CACAACAA | GUUAUGGU | UUCCUUAGCAG |
| | | | | GTCTG | ATGT | CTGAACTGTACC | AGUCUG | ACAUGU | AGCUGAACUGU |
| | | | | [SEQ ID | [SEQ ID | ACAACAAAGTC | [SEQ ID | [SEQ ID | ACCACAACAAA |
| | | | | NO: 652] | NO: 1701] | TGTGTCTAAACT | NO: 1180] | NO: 1896] | GUCUGUGUCUA |
| | | | | | | ATCAGACTTTGT | | | AACUAUCAGAC |
| | | | | | | TATGGTACATGT | | | UUUGUUAUGGU |
| | | | | | | TAGCTACTGCTA | | | ACAUGUUAGCU |
| | | | | | | GGCAATCCTTCC | | | ACUGCUAGGCA |
| | | | | | | CTCGATAAATGT | | | AUCCUUCCCUC |
| | | | | | | CTTGGCATCGTT | | | GAUAAAUGUCU |
| | | | | | | TGCTTTG | | | UGGCAUCGUUU |
| | | | | | | [SEQ ID NO: 1794] | | | GCUUUG |
| | | | | | | | | | [SEQ ID NO: 1991] |
| 3301 | miR-122_M | -2.26423 | 0.053866 | TGAACAAG | TCCCAAATC | GGCTACAGAGTT | UGAACAAG | UCCCAAAU | GGCUACAGAGU |
| | | | | GGGCTGATT | AGACCCTTG | TGCTTAGCAGAG | GGGCUGAU | CAGACCCU | UUGCUUAGCAG |
| | | | | TGGGA | TGAA | CTGTGAACAAG | UUGGGA | UGUGAA | AGCUGUGAACA |
| | | | | [SEQ ID | [SEQ ID | GGGCTGATTTGG | [SEQ ID | SEQ ID | AGGGGCUGAUU |
| | | | | NO: 687] | NO: 1702] | GATGTCTAAACT | NO: 1215] | NO: 1897] | UGGGAUGUCUA |
| | | | | | | ATTCCCAAATCA | | | AACUAUUCCCA |
| | | | | | | GACCCTTGTGAA | | | AAUCAGACCCU |
| | | | | | | TAGCTACTGCTA | | | UGUGAAUAGCU |
| | | | | | | GGCCATCCTTCC | | | ACUGCUAGGCC |
| | | | | | | CTCGATAAATGT | | | AUCCUUCCCUC |
| | | | | | | CTTGGCATCGTT | | | GAUAAAUGUCU |
| | | | | | | TGCTTTG | | | UGGCAUCGUUU |
| | | | | | | [SEQ ID NO: 1795] | | | GCUUUG |
| | | | | | | | | | [SEQ ID NO: 1992] |
| 3341 | miR- | -2.26383 | 0.394316 | ATAGACAT | GTCAGCATC | GAGCTCAGTCA | AUAGACAU | GUCAGCAU | GAGCUCAGUCA |
| | 190a_M | | | GAGGATGC | CTCATGTCT | AACCTGGATGCC | GAGGAUGC | CCUCAUGU | AACCUGGAUGC |
| | | | | TGAGAC | CG | TTTTCTGCAGGC | UGAGAC | CUCG | CUUUUCUGCAG |
| | | | | [SEQ ID | [SEQ ID | GTCTGTGATAGA | [SEQ ID | [SEQ ID | GCGUCUGUGAU |
| | | | | NO: 1617] | NO: 1703] | CATGAGGATGCT | NO: 1813] | NO: 1898] | AGACAUGAGGA |
| | | | | | | GAGACTGTTATT | | | UGCUGAGACUG |
| | | | | | | TAATCCAGTCAG | | | UUAUUUAAUCC |
| | | | | | | CATCCTCATGTC | | | AGUCAGCAUCC |
| | | | | | | TCGCTACAGTCT | | | UCAUGUCUCGC |
| | | | | | | CTTGCCCTGTCT | | | UACAGUCUCUU |
| | | | | | | CCGGGGGTTCCT | | | GCCCUGUCUCC |
| | | | | | | AATAAAG | | | GGGGGUUCCUA |
| | | | | | | [SEQ ID NO: 1796] | | | AUAAAG |
| | | | | | | | | | [SEQ ID NO: 1993] |
| 2945 | miR-100_M | -2.26187 | 0.044364 | TGTAGTAG | TCAGCCCA | CCCAAAAGAGA | UGUAGUAG | UCAGCCCA | CCCAAAAGAGA |
| | | | | AAGGCTTTG | AGACTTCTA | GAAGATATTGAT | AAGGCUUU | AGACUUCU | GAAGAUAUUGA |
| | | | | GCTGA | ATACT | GCCTGTTGCCAC | GGCUGA | AAUACU | UGCCUGUUGCC |
| | | | | [SEQ ID | [SEQ ID | ATGTAGTAGAA | [SEQ ID | [SEQ ID | ACAUGUAGUAG |
| | | | | NO: 685] | NO: 1678] | GGCTTTGGCTGA | NO: 1213] | NO: 1873] | AAGGCUUUGGC |
| | | | | | | GTATTAGTCCGT | (Same guide as | | UGAGUAUUAGU |
| | | | | | | CAGCCCAAGAC | XD-14860) | | CCGUCAGCCCA |
| | | | | | | TTCTAATACTTG | | | AGACUUCUAAU |
| | | | | | | TGTCTGTTAGGC | | | ACUUGUGUCUG |
| | | | | | | TATTCCACGGAC | | | UUAGGCUAUUC |
| | | | | | | CTGGGGCTTTGC | | | CACGGACCUGG |
| | | | | | | TTATATGCC | | | GGCUUUGCUUA |
| | | | | | | [SEQ ID NO: 1797] | | | UAUGCC |
| | | | | | | | | | [SEQ ID NO: 1994] |
| 1756 | miR-1-1_M | -2.26119 | 0.153277 | TTCGGGTTG | AGACTTCA | CATGCAGACTGC | UUCGGGUU | AGACUUCA | CAUGCAGACUG |
| | | | | AAATCTGA | GATTTCAAC | CTGCTTGGGAGA | GAAAUCUG | GAUUUCAA | CCUGCUUGGGA |
| | | | | AGTGT | CGAGAA | CTTCAGATTTCA | AAGUGU | CCGAGAA | GACUUCAGAUU |
| | | | | [SEQ ID | [SEQ ID | ACCGAGAATAT | [SEQ ID | [SEQ ID | UCAACCGAGAA |
| | | | | NO: 607] | NO: 1704] | GGACCTGCTAA | NO: 108] | NO: 1907] | UAUGGACCUGC |
| | | | | | | GCTATTCGGGTT | (Same guide as | | UAAGCUAUUCG |
| | | | | | | GAAATCTGAAG | XD-14790) | | GGUUGAAAUCU |
| | | | | | | TGTCTCAGGCCG | | | GAAGUGUCUCA |
| | | | | | | GGACCTCTTCCG | | | GGCCGGGACCU |
| | | | | | | CCGCACTGAGG | | | CUUCCGCCGCA |
| | | | | | | GGCACTCCACAC | | | CUGAGGGGCAC |
| | | | | | | CACGGGGGCC | | | UCCACACCACG |
| | | | | | | [SEQ ID NO: 1798] | | | GGGGCC |
| | | | | | | | | | [SEQ ID NO: 1995] |
| 3845 | miR-1-1_M | -2.26073 | -0.20695 | TTGAACGTG | GTTCCATCC | CATGCAGACTGC | UUGAACGU | GUUCCAUC | CAUGCAGACUG |
| | | | | AGAAGGAT | TTCTCACGT | CTGCTTGGGGTT | GAGAAGGA | CUUCUCAC | CCUGCUUGGGG |
| | | | | GGATC | CGCAA | CCATCCTTCTCA | UGGAUC | GUCGCAA | UUCCAUCCUUC |
| | | | | [SEQ ID | [SEQ ID | CGTCGCAATATG | [SEQ ID | [SEQ ID | UCACGUCGCAA |
| | | | | NO: 696] | NO: 1705] | GACCTGCTAAGC | NO: 1224] | NO: 1906] | UAUGGACCUGC |
| | | | | | | TATTGAACGTGA | | | UAAGCUAUUGA |
| | | | | | | GAAGGATGGAT | | | ACGUGAGAAGG |
| | | | | | | CCTCAGGCCGG | | | AUGGAUCCUCA |
| | | | | | | GACCTCTTCCGC | | | GGCCGGGACCU |
| | | | | | | CGCACTGAGGG | | | CUUCCGCCGCA |
| | | | | | | GCACTCCACACC | | | CUGAGGGGCAC |
| | | | | | | ACGGGGGCC | | | UCCACACCACG |
| | | | | | | [SEQ ID NO: 1799] | | | GGGGCC |
| | | | | | | | | | [SEQ ID NO: 1996] |
| 3043 | miR-190a | -2.26041 | 0.277711 | TTTGGTGCA | AGCGTTTGT | GAGCTCAGTCA | UUUGGUGC | AGCGUUUG | GAGCUCAGUCA |
| | | | | AAACAAAC | TTTGCACCA | AACCTGGATGCC | AAAACAAA | UUUUGCAC | AACCUGGAUGC |
| | | | | AGGCT | CC | TTTTCTGCAGGC | CAGGCU | CACC | CUUUUCUGCAG |
| | | | | [SEQ ID | [SEQ ID | CTCTGTGTTTGG | [SEQ ID | [SEQ ID | GCCUCUGUGUU |
| | | | | NO: 1615] | NO: 1664] | TGCAAAACAAA | NO: 1812] | NO: 1859] | UGGUGCAAAAC |
| | | | | | | CAGGCTTGTTAT | | | AAACAGGCUUG |
| | | | | | | TTAATCCAAGCG | | | UUAUUUAAUCC |
| | | | | | | TTTGTTTTGCAC | | | AAGCGUUUGUU |
| | | | | | | CACCCTACAGTG | | | UUGCACCACCC |
| | | | | | | TCTTGCCCTGTC | | | UACAGUGUCUU |
| | | | | | | TCCGGGGGTTCC | | | GCCCUGUCUCC |
| | | | | | | TAATAAAG | | | GGGGGUUCCUA |
| | | | | | | [SEQ ID NO: 1800] | | | AUAAAG |
| | | | | | | | | | [SEQ ID NO: 1997] |
| 1162 | miR-155E | -2.26036 | -0.20831 | AACTGTACC | CAGACTTTT | CTGGAGGCTTGC | AACUGUAC | CAGACUUU | CUGGAGGCUUG |
| | | | | ACAACAAA | TGGGTACA | TTTGGGCTGTAT | CACAACAA | UUGGGUAC | CUUUGGGCUGU |
| | | | | GTCTG | GTT | GCTGAACTGTAC | AGUCUG | AGUU | AUGCUGAACUG |
| | | | | [SEQ ID | [SEQ ID | CACAACAAAGT | [SEQ ID | [SEQ ID | UACCACAACAA |
| | | | | NO: 652] | NO: 1706] | CTGTTTTGGCCT | NO: 1180] | NO: 1905] | AGUCUGUUUUG |
| | | | | | | CTGACTGACAG | | | GCCUCUGACUG |
| | | | | | | ACTTTTTGGGTA | | | ACAGACUUUUU |
| | | | | | | CAGTTCAGGAC | | | GGGUACAGUUC |
| | | | | | | AAGGCCCTTTAT | | | AGGACAAGGCC |
| | | | | | | CAGCACTCACAT | | | CUUUAUCAGCA |
| | | | | | | GGAACAAATGG | | | CUCACAUGGAA |
| | | | | | | CCACCGTGGG | | | CAAAUGGCCAC |
| | | | | | | [SEQ ID NO: 1801] | | | CGUGGG |
| | | | | | | | | | [SEQ ID NO: 1998] |
| 3273 | miR-1-1_M | -2.24883 | 0.09481 | TAGGACTGT | CTATATGTT | CATGCAGACTGC | UAGGACUG | CUAUAUGU | CAUGCAGACUG |
| | | | | AGGCAACA | GCCTACAGT | CTGCTTGGGCTA | UAGGCAAC | UGCCUACA | CCUGCUUGGGC |
| | | | | TATTG | GACTA | TATGTTGCCTAC | AUAUUG | GUGACUA | UAUAUGUUGCC |
| | | | | [SEQ ID | [SEQ ID | AGTGACTATATG | [SEQ ID | [SEQ ID | UACAGUGACUA |
| | | | | NO: 1628] | NO: 1691] | GACCTGCTAAGC | NO: 1821] | NO: 1886] | UAUGGACCUGC |
| | | | | | | TATAGGACTGTA | | | UAAGCUAUAGG |
| | | | | | | GGCAACATATTG | | | ACUGUAGGCAA |
| | | | | | | CTCAGGCCGGG | | | CAUAUUGCUCA |
| | | | | | | ACCTCTTCCGCC | | | GGCCGGGACCU |
| | | | | | | GCACTGAGGGG | | | CUUCCGCCGCA |
| | | | | | | CACTCCACACCA | | | CUGAGGGGCAC |
| | | | | | | CGGGGGCC | | | UCCACACCACG |
| | | | | | | [SEQ ID NO: 1802] | | | GGGGCC |
| | | | | | | | | | [SEQ ID NO: 1999] |
| 2586 | miR-190a | -2.24846 | 0.172174 | TAGATTCAG | CCATTCTAC | GAGCTCAGTCA | UAGAUUCA | CCAUUCUA | GAGCUCAGUCA |
| | | | | AAGTAGAA | TTCTGAATC | AACCTGGATGCC | GAAGUAGA | CUUCUGAA | AACCUGGAUGC |
| | | | | CTTGG | CC | TTTTCTGCAGGC | ACUUGG | UCCC | CUUUUCUGCAG |
| | | | | [SEQ ID | [SEQ ID | CTCTGTGTAGAT | SEQ ID | [SEQ ID | GCCUCUGUGUA |
| | | | | NO: 1621] | NO: 1661] | TCAGAAGTAGA | NO: 1816] | NO: 1856] | GAUUCAGAAGU |
| | | | | | | ACTTGGTGTTAT | | | AGAACUUGGUG |
| | | | | | | TTAATCCACCAT | | | UUAUUUAAUCC |
| | | | | | | TCTACTTCTGAA | | | ACCAUUCUACU |
| | | | | | | TCCCCTACAGTG | | | UCUGAAUCCCC |
| | | | | | | TCTTGCCCTGTC | | | UACAGUGUCUU |
| | | | | | | TCCGGGGGTTCC | | | GCCCUGUCUCC |
| | | | | | | TAATAAAG | | | |
| | | | | | | [SEQ ID NO: 1803] | | | GGGGGUUCCUA |
| | | | | | | | | | AUAAAG |
| | | | | | | | | | [SEQ ID NO: 2000] |
| 3132 | miR-122 M | -2.24518 | 0.049515 | ATGTCTTGG | CCAGTGAA | GGCTACAGAGTT | AUGUCUUG | CCAGUGAA | GGCUACAGAGU |
| | | | | CTTGATTCA | TCACGCCA | TGCTTAGCAGAG | GCUUGAUU | UCACGCCA | UUGCUUAGCAG |
| | | | | CTGG | AGAACT | CTGATGTCTTGG | CACUGG | AGAACU | AGCUGAUGUCU |
| | | | | [SEQ ID | [SEQ ID | CTTGATTCACTG | [SEQ ID | [SEQ ID | UGGCUUGAUUC |
| | | | | NO: 1631] | NO: 1707] | GTGTCTAAACTA | NO: 1826] | NO: 1904] | ACUGGUGUCUA |
| | | | | | | TCCAGTGAATCA | | | AACUAUCCAGU |
| | | | | | | CGCCAAGAACTT | | | GAAUCACGCCA |
| | | | | | | AGCTACTGCTAG | | | AGAACUUAGCU |
| | | | | | | GCCATCCTTCCC | | | ACUGCUAGGCC |
| | | | | | | TCGATAAATGTC | | | AUCCUUCCCUC |
| | | | | | | TTGGCATCGTTT | | | GAUAAAUGUCU |
| | | | | | | GCTTTG | | | UGGCAUCGUUU |
| | | | | | | [SEQ ID NO: 1804] | | | GCUUUG |
| | | | | | | | | | [SEQ ID NO: 2001] |
| 3842 | miR-1-1_M | -2.24315 | -0.38269 | AACGTGAG | TTCGATCCA | CATGCAGACTGC | AACGUGAG | UUCGAUCC | CAUGCAGACUG |
| | | | | AAGGATGG | TCCTTCTCA | CTGCTTGGGTTC | AAGGAUGG | AUCCUUCU | CCUGCUUGGGU |
| | | | | ATCGTA | GAGTT | GATCCATCCTTC | AUCGUA | CAGAGUU | UCGAUCCAUCC |
| | | | | [SEQ ID | [SEQ ID | TCAGAGTTTATG | [SEQ ID | [SEQ ID | UUCUCAGAGUU |
| | | | | NO: 1625] | NO: 1699] | GACCTGCTAAGC | NO: 1824] | NO: 1894] | UAUGGACCUGC |
| | | | | | | TAAACGTGAGA | | | UAAGCUAAACG |
| | | | | | | AGGATGGATCG | | | UGAGAAGGAUG |
| | | | | | | TACTCAGGCCGG | | | GAUCGUACUCA |
| | | | | | | GACCTCTTCCGC | | | GGCCGGGACCU |
| | | | | | | CGCACTGAGGG | | | CUUCCGCCGCA |
| | | | | | | GCACTCCACACC | | | CUGAGGGGCAC |
| | | | | | | ACGGGGGCC | | | UCCACACCACG |
| | | | | | | [SEQ ID NO: 1805] | | | GGGGCC |
| | | | | | | | | | [SEQ ID NO: 2002] |
| 2928 | miR-1-1 | -2.24149 | 0.19907 | TCTGAGAG | ATCCCACGT | CATGCAGACTGC | UCUGAGAG | AUCCCACG | CAUGCAGACUG |
| | | | | AAGGAACG | TCCTTCTCT | CTGCTTGGGATC | AAGGAACG | UUCCUUCU | CCUGCUUGGGA |
| | | | | TGGGTT | GAAGA | CCACGTTCCTTC | UGGGUU | CUGAAGA | UCCCACGUUCC |
| | | | | [SEQ ID | [SEQ ID | TCTGAAGATATG | [SEQ ID | [SEQ ID | UUCUCUGAAGA |
| | | | | NO: 668] | NO: 1708] | GACCTGCTAAGC | NO: 1196] | NO: 1902] | UAUGGACCUGC |
| | | | | | | TATCTGAGAGA | | | UAAGCUAUCUG |
| | | | | | | AGGAACGTGGG | | | AGAGAAGGAAC |
| | | | | | | TTCTCAGGCCGG | | | GUGGGUUCUCA |
| | | | | | | GACCTCTCTCGC | | | GGCCGGGACCU |
| | | | | | | CGCACTGAGGG | | | CUCUCGCCGCA |
| | | | | | | GCACTCCACACC | | | CUGAGGGGCAC |
| | | | | | | ACGGGGGCC | | | UCCACACCACG |
| | | | | | | [SEQ ID NO: 1806] | | | GGGGCC |
| | | | | | | | | | [SEQ ID NO: 2003] |
| 1231 | miR-1-1_M | -2.24102 | 0.10817 | TTCACTTTA | CAGCTATCA | CATGCAGACTGC | UUCACUUU | CAGCUAUC | CAUGCAGACUG |
| | | | | GCACTGAT | GTGCTAAA | CTGCTTGGGCAG | AGCACUGA | AGUGCUAA | CCUGCUUGGGC |
| | | | | AGCAG | GCGGAA | CTATCAGTGCTA | UAGCAG | AGCGGAA | AGCUAUCAGUG |
| | | | | [SEQ ID | [SEQ ID | AAGCGGAATAT | [SEQ ID | [SEQ ID | CUAAAGCGGAA |
| | | | | NO: 1627] | NO: 1709] | GGACCTGCTAA | NO: 1825] | NO: 1903] | UAUGGACCUGC |
| | | | | | | GCTATTCACTTT | | | UAAGCUAUUCA |
| | | | | | | AGCACTGATAG | | | CUUUAGCACUG |
| | | | | | | CAGCTCAGGCC | | | AUAGCAGCUCA |
| | | | | | | GGGACCTCTTCC | | | GGCCGGGACCU |
| | | | | | | GCCGCACTGAG | | | CUUCCGCCGCA |
| | | | | | | GGGCACTCCAC | | | CUGAGGGGCAC |
| | | | | | | ACCACGGGGGC | | | UCCACACCACG |
| | | | | | | C | | | GGGGCC |
| | | | | | | [SEQ ID NO: 1807] | | | [SEQ ID NO: 2004] |
| 1578 | miR-100 M | -2.24051 | -0.33444 | TGGAATTTC | ACAGCAAT | CCCAAAAGAGA | UGGAAUUU | ACAGCAAU | CCCAAAAGAGA |
| | | | | TCTGAACTG | TCCGAGAA | GAAGATATTGAT | CUCUGAAC | UCCGAGAA | GAAGAUAUUGA |
| | | | | CTGT | ACTCCT | GCCTGTTGCCAC | UGCUGU | ACUCCU | UGCCUGUUGCC |
| | | | | [SEQ ID | [SEQ ID | ATGGAATTTCTC | [SEQ ID | [SEQ ID | ACAUGGAAUUU |
| | | | | NO: 1626] | NO: 1665] | TGAACTGCTGTG | NO: 1820] | NO: 1860] | CUCUGAACUGC |
| | | | | | | TATTAGTCCGAC | | | UGUGUAUUAGU |
| | | | | | | AGCAATTCCGA | | | CCGACAGCAAU |
| | | | | | | GAAACTCCTTGT | | | UCCGAGAAACU |
| | | | | | | GTCTGTTAGGCT | | | CCUUGUGUCUG |
| | | | | | | ATTCCACGGACC | | | UUAGGCUAUUC |
| | | | | | | TGGGGCTTTGCT | | | CACGGACCUGG |
| | | | | | | TATATGCC | | | GGCUUUGCUUA |
| | | | | | | [SEQ ID NO: 1808] | | | UAUGCC |
| | | | | | | | | | [SEQ ID NO: 2005] |
| 967 | miR-190a | -2.23671 | -0.08895 | ACTGATGTA | TGGCATATA | GAGCTCAGTCA | ACUGAUGU | UGGCAUAU | GAGCUCAGUCA |
| | | | | AGTATATG | CTTACATCA | AACCTGGATGCC | AAGUAUAU | ACUUACAU | AACCUGGAUGC |
| | | | | AACCA | AG | TTTTCTGCAGGC | GAACCA | CAAG | CUUUUCUGCAG |
| | | | | [SEQ ID | [SEQ ID | CTCTGTGACTGA | [SEQ ID | [SEQ ID | GCCUCUGUGAC |
| | | | | NO: 1619] | NO: 1642] | TGTAAGTATATG | NO: 1815] | NO: 1837] | UGAUGUAAGUA |
| | | | | | | AACCATGTTATT | | | UAUGAACCAUG |
| | | | | | | TAATCCATGGCA | | | UUAUUUAAUCC |
| | | | | | | TATACTTACATC | | | AUGGCAUAUAC |
| | | | | | | AAGCTACAGTGT | | | UUACAUCAAGC |
| | | | | | | CTTGCCCTGTCT | | | UACAGUGUCUU |
| | | | | | | CCGGGGGTTCCT | | | GCCCUGUCUCC |
| | | | | | | AATAAAG | | | GGGGGUUCCUA |
| | | | | | | [SEQ ID NO: 1809] | | | AUAAAG |
| | | | | | | | | | [SEQ ID NO: 2006] |
| 1436 | miR155-M | -2.22884 | 0.141256 | TGAGTTATC | GCCCTTAGA | CCTGGAGGCTTG | UGAGUUAU | GCCCUUAG | CCUGGAGGCUU |
| | | | | TCTTTCTAA | AGGATAAC | CTGAAGGCTGTA | CUCUUUCU | AAGGAUAA | GCUGAAGGCUG |
| | | | | GGGC | TCA | TGCTGTGAGTTA | AAGGGC | CUCA | UAUGCUGUGAG |
| | | | | [SEQ ID | [SEQ ID | TCTCTTTCTAAG | [SEQ ID | [SEQ ID | UUAUCUCUUUC |
| | | | | NO: 1632] | NO: 1710] | GGCTTTTGGCCA | NO: 1827] | NO: 1900] | UAAGGGCUUUU |
| | | | | | | CTGACTGAGCCC | | | GGCCACUGACU |
| | | | | | | TTAGAAGGATA | | | GAGCCCUUAGA |
| | | | | | | ACTCACAGGAC | | | AGGAUAACUCA |
| | | | | | | ACAAGGCCTGTT | | | CAGGACACAAG |
| | | | | | | ACTAGCACTCAC | | | GCCUGUUACUA |
| | | | | | | ATGGAACAAAT | | | GCACUCACAUG |
| | | | | | | GGCCACCGG | | | GAACAAAUGGC |
| | | | | | | [SEQ ID NO: 1810] | | | CACCGG |
| | | | | | | | | | [SEQ ID NO: 2007] |

TABLE 24
Top 10 miRNAs for each miR backbone

| | Atxn2 | | | | | | | | |
|---|---|---|---|---|---|---|---|---|---|
| | Tar- | Atxn2 | | | | | | | |
| | get- | low/ | | Guide | | | | | |
| miR | ing | unsort | T1/T0 | Se- | Passenger | MiR | Guide | Passenger | MiR |
| Back- | Posi- | log2 | log2 | quence | Sequence | Cassette | Sequence | Sequence | Cassette |
| bone | tion | FC | FC | (DNA) | (DNA) | (DNA) | (RNA) | (RNA) | (RNA) |
| miR- | 1755 | −2.5734 | −0.10352 | TCGGGTTGAA | CTCACTTCAG | CATGCAGACT | UCGGGUUGAA | CUCACUUCAG | CAUGCAGACU |
| 1-1 | | | | ATCTGAAGTG | ATTTCAACGA | GCCTGCTTGG | AUCUGAAGUG | AUUUCAACGA | GCCUGCUUGG |
| | | | | TG | CGA | GCTCACTTCA | UG | CGA | GCUCACUUCA |
| | | | | [SEQ ID | [SEQ ID | GATTTCAACG | [SEQ ID | [SEQ ID | GAUUUCAACG |
| | | | | NO: | NO: | ACGATATGGA | NO: | NO: | ACGAUAUGGA |
| | | | | 657] | 1645] | CCTGCTAAGC | 1185] | 1840] | CCUGCUAAGC |
| | | | | | | TATCGGGTTG | | | UAUCGGGUUG |
| | | | | | | AAATCTGAAG | | | AAAUCUGAAG |
| | | | | | | TGTGCTCAGG | | | UGUGCUCAGG |
| | | | | | | CCGGGACCTC | | | CCGGGACCUC |
| | | | | | | TCTCGCCGCA | | | UCUCGCCGCA |
| | | | | | | CTGAGGGGCA | | | CUGAGGGGCA |
| | | | | | | CTCCACACCA | | | CUCCACACCA |
| | | | | | | CGGGGGCC | | | CGGGGGCC |
| | | | | | | [SEQ ID | | | [SEQ ID |
| | | | | | | NO: | | | NO: |
| | | | | | | 1726] | | | 1923] |
| miR- | 2943 | −2.54936 | 0.148183 | TAGTAGAAGG | TGTCAGCCAA | CATGCAGACT | UAGUAGAAGG | UGUCAGCCAA | CAUGCAGACU |
| 1-1 | | | | CTTTGGCTGA | AGCCTTCTCC | GCCTGCTTGG | CUUUGGCUGA | AGCCUUCUCC | GCCUGCUUGG |
| | | | | GA | CTA | GTGTCAGCCA | GA | CUA | GUGUCAGCCA |
| | | | | [SEQ ID | [SEQ ID | AAGCCTTCTC | [SEQ ID | [SEQ ID | AAGCCUUCUC |
| | | | | NO: | NO: | CCTATATGGA | NO: | NO: | CCUAUAUGGA |
| | | | | 683] | 1650] | CCTGCTAAGC | 1211] | 1845] | CCUGCUAAGC |
| | | | | | | TATAGTAGAA | | | UAUAGUAGAA |
| | | | | | | GGCTTTGGCT | | | GGCUUUGGCU |
| | | | | | | GAGACTCAGG | | | GAGACUCAGG |
| | | | | | | CCGGGACCTC | | | CCGGGACCUC |
| | | | | | | TCTCGCCGCA | | | UCUCGCCGCA |
| | | | | | | CTGAGGGGCA | | | CUGAGGGGCA |
| | | | | | | CTCCACACCA | | | CUCCACACCA |
| | | | | | | CGGGGGCC | | | CGGGGGCC |
| | | | | | | [SEQ ID | | | [SEQ ID |
| | | | | | | NO: | | | NO: |
| | | | | | | 1731] | | | 1928] |
| miR- | 3338 | −2.52716 | −0.2585 | TACATGAGGA | TGAGTCTCAG | CATGCAGACT | UACAUGAGGA | UGAGUCUCAG | CAUGCAGACU |
| 1-1 | | | | TGCTGAGACT | CATCCTCACG | GCCTGCTTGG | UGCUGAGACU | CAUCCUCACG | GCCUGCUUGG |
| | | | | GA | GTA | GTGAGTCTCA | GA | GUA | GUGAGUCUCA |
| | | | | [SEQ ID | [SEQ ID | GCATCCTCAC | [SEQ ID | [SEQ ID | GCAUCCUCAC |
| | | | | NO: | NO: | GGTATATGGA | NO: | NO: | GGUAUAUGGA |
| | | | | 1620] | 1651] | CCTGCTAAGC | 314] | 1846] | CCUGCUAAGC |
| | | | | | | TATACATGAG | (Same | | UAUACAUGAG |
| | | | | | | GATGCTGAGA | guide | | GAUGCUGAGA |
| | | | | | | CTGACTCAGG | as | | CUGACUCAGG |
| | | | | | | CCGGGAC | XD- | | CCGGGACCUC |
| | | | | | | CTCT | 14893 | | U |
| | | | | | | CTCGCC | | | CUCGCCGCAC |
| | | | | | | GCACTGAGGG | | | UGAGGGGCAC |
| | | | | | | GCACTCCACA | | | UCCACACCAC |
| | | | | | | CCACGGGGGC | | | GGGGGCC |
| | | | | | | C | | | [SEQ ID |
| | | | | | | [SEQ ID | | | NO: |
| | | | | | | NO: | | | 1929] |
| | | | | | | 1732] | | | |
| miR- | 3302 | −2.51939 | −0.06675 | TTGAACAAGG | CGCAAATCAG | CATGCAGACT | UUGAACAAGG | CGCAAAUCAG | CAUGCAGACU |
| 1-1 | | | | GGCTGATTTG | CCCCTTGTCG | GCCTGCTTGG | GGCUGAUUUG | CCCCUUGUCG | GCCUGCUUGG |
| | | | | GG | CAA | GCGCAAATCA | GG | CAA | GCGCAAAUCA |
| | | | | [SEQ ID | [SEQ ID | GCCCCTTGTC | [SEQ ID | [SEQ ID | GCCCCUUGUC |
| | | | | NO: | NO: | GCAATATGGA | NO: | NO: | GCAAUAUGGA |
| | | | | 688] | 1647] | CCTGCTAAGC | 1216] | 1842] | CCUGCUAAGC |
| | | | | | | TATTGAACAA | | | UAUUGAACAA |
| | | | | | | GGGGCTGATT | | | GGGGCUGAUU |
| | | | | | | TGGGCTCAGG | | | UGGGCUCAGG |
| | | | | | | CCGGGACCTC | | | CCGGGACCUC |
| | | | | | | TCTCGCCGCA | | | UCUCGCCGCA |
| | | | | | | CTGAGGGGCA | | | CUGAGGGGCA |
| | | | | | | CTCCACACCA | | | CUCCACACCA |
| | | | | | | CGGGGGCC | | | CGGGGGCC |
| | | | | | | [SEQ ID | | | [SEQ ID |
| | | | | | | NO: | | | NO: |
| | | | | | | 1735] | | | 1932] |
| miR- | 3341 | −2.50291 | −0.1115 | ATAGACATGA | GACTCAGCAT | CATGCAGACT | AUAGACAUGA | GACUCAGCAU | CAUGCAGACU |
| 1-1 | | | | GGATGCTGAG | CCTCATGTGA | GCCTGCTTGG | GGAUGCUGAG | CCUCAUGUGA | GCCUGCUUGG |
| | | | | AC | TAT | GGACTCAGCA | AC | UAU | GGACUCAGCA |
| | | | | [SEQ ID | [SEQ ID | TCCTCATGTG | [SEQ ID | [SEQ ID | UCCUCAUGUG |
| | | | | NO: | NO: | ATATTATGGA | NO: | NO: | AUAUUAUGGA |
| | | | | 1617] | 1637] | CCTGCTAAGC | 1813] | 1832] | CCUGCUAAGC |
| | | | | | | TAATAGACAT | | | UAAUAGACAU |
| | | | | | | GAGGATGCTG | | | GAGGAUGCUG |
| | | | | | | AGACCTCAGG | | | AGACCUCAGG |
| | | | | | | CCGGGACCTC | | | CCGGGACCUC |
| | | | | | | TCTCGCCGCA | | | UCUCGCCGCA |
| | | | | | | CTGAGGGGCA | | | CUGAGGGGCA |
| | | | | | | CTCCACACCA | | | CUCCACACCA |
| | | | | | | CGGGGGCC | | | CGGGGGCC |
| | | | | | | [SEQ ID | | | [SEQ ID |
| | | | | | | NO: | | | NO: |
| | | | | | | 1737] | | | 1934] |
| miR- | 2945 | −2.42094 | −0.06694 | TGTAGTAGAA | TGAGCCAAAG | CATGCAGACT | UGUAGUAGAA | UGAGCCAAAG | CAUGCAGACU |
| 1-1 | | | | GGCTTTGGCT | CCTTCTACCG | GCCTGCTTGG | GGCUUUGGCU | CCUUCUACCG | GCCUGCUUGG |
| | | | | GA | ACA | GTGAGCCAAA | GA | ACA | GUGAGCCAAA |
| | | | | [SEQ ID | [SEQ ID | GCCTTCTACC | [SEQ ID | [SEQ ID | GCCUUCUACC |
| | | | | NO: | NO: | GACATATGGA | NO: | NO: | GACAUAUGGA |
| | | | | 685] | 1633] | CCTGCTAAGC | 1213] | 1828] | CCUGCUAAGC |
| | | | | | | TATGTAGTAG | (Same | | UAUGUAGUAG |
| | | | | | | AAGGCTTTGG | guide | | AAGGCUUUGG |
| | | | | | | CTGACTCAGG | as | | CUGACUCAGG |
| | | | | | | CCGGGACCTC | XD- | | CCGGGACCUC |
| | | | | | | TCTCGCCGCA | 14860) | | UCUCGCCGCA |
| | | | | | | C | | | CUGAG |
| | | | | | | TGAGGGGCAC | | | GGGCACUCCA |
| | | | | | | TCCACACCAC | | | CACCACGGGG |
| | | | | | | GGGGGCC | | | GCC |
| | | | | | | [SEQ ID | | | [SEQ ID |
| | | | | | | NO: | | | NO: |
| | | | | | | 1761] | | | 1958] |
| miR- | 3301 | −2.41082 | 0.051034 | TGAACAAGGG | TGCCAAATCA | CATGCAGACT | UGAACAAGGG | UGCCAAAUCA | CAUGCAGACU |
| 1-1 | | | | GCTGATTTGG | GCCCCTTGCG | GCCTGCTTGG | GCUGAUUUGG | GCCCCUUGCG | GCCUGCUUGG |
| | | | | GA | TCA | GTGCCAAATC | GA | UCA | GUGCCAAAUC |
| | | | | [SEQ ID | [SEQ ID | AGCCCCTTGC | [SEQ ID | [SEQ ID | AGCCCCUUGC |
| | | | | NO: | NO: | GTCATATGGA | NO: | NO: | GUCAUAUGGA |
| | | | | 687] | 1673] | CCTGCTAAGC | 1215] | 1868] | CCUGCUAAGC |
| | | | | | | TATGAACAAG | | | UAUGAACAAG |
| | | | | | | GGGCTGATTT | | | GGGCUGAUUU |
| | | | | | | GGGACTCAGG | | | GGGACUCAGG |
| | | | | | | CCGGGACCTC | | | CCGGGACCUC |
| | | | | | | TCTCGCCGCA | | | UCUCGCCGCA |
| | | | | | | CTGAGGGGCA | | | CUGAGGGGCA |
| | | | | | | CTCCACACCA | | | CUCCACACCA |
| | | | | | | CGGGGGCC | | | CGGGGGCC |
| | | | | | | [SEQ ID | | | [SEQ ID |
| | | | | | | NO: | | | NO: |
| | | | | | | 1763] | | | 1960] |
| miR- | 3043 | −2.32683 | 0.097086 | TTTGGTGCAA | ACCCTGTTTG | CATGCAGACT | UUUGGUGCAA | ACCCUGUUUG | CAUGCAGACU |
| 1-1 | | | | AACAAACAGG | TTTTGCACGA | GCCTGCTTGG | AACAAACAGG | UUUUGCACGA | GCCUGCUUGG |
| | | | | CT | AAA | GACCCTGTTT | CU | AAA | GACCCUGUUU |
| | | | | [SEQ ID | [SEQ ID | GTTTTGCACG | [SEQ ID | [SEQ ID | GUUUUGCACG |
| | | | | NO: | NO: | AAAATATGGA | NO: | NO: | AAAAUAUGGA |
| | | | | 1615] | 1690] | CCTGCTAAGC | 1812] | 1885] | CCUGCUAAGC |
| | | | | | | TATTTGGTGC | | | UAUUUGGUGC |
| | | | | | | AAAACAAACA | | | AAAACAAACA |
| | | | | | | GGCTCTCAGG | | | GGCUCUCAGG |
| | | | | | | CCGGGACCTC | | | CCGGGACCUC |
| | | | | | | TCTCGCCGCA | | | UCUCGCCGCA |
| | | | | | | CTGAGGGGCA | | | CUGAGGGGCA |
| | | | | | | CTCCACACCA | | | CUCCACACCA |
| | | | | | | CGGGGGcc | | | CGGGGGCC |
| | | | | | | [SEQ ID | | | [SEQ ID |
| | | | | | | NO: | | | NO: |
| | | | | | | 1783] | | | 1980] |
| miR- | 3273 | −2.32464 | −0.13929 | TAGGACTGTA | CTATATGTTG | CATGCAGACT | UAGGACUGUA | CUAUAUGUUG | CAUGCAGACU |
| 1-1 | | | | GGCAACATAT | CCTACAGTGA | GCCTGCTTGG | GGCAACAUAU | CCUACAGUGA | GCCUGCUUGG |
| | | | | TG | CTA | GCTATATGTT | UG | CUA | GCUAUAUGUU |
| | | | | [SEQ ID | [SEQ ID | GCCTACAGTG | [SEQ ID | [SEQ ID | GCCUACAGUG |
| | | | | NO: | NO: | ACTATATGGA | NO: | NO: | ACUAUAUGGA |
| | | | | 1628] | 1691] | CCTGCTAAGC | 1821] | 1886] | CCUGCUAAGC |
| | | | | | | TATAGGACTG | | | UAUAGGACUG |
| | | | | | | TAGGCAACAT | | | UAGGCAACAU |
| | | | | | | ATTGCTCAGG | | | AUUGCUCAGG |
| | | | | | | CCGGGACCTC | | | CCGGGACCUC |
| | | | | | | TCTCGCCGCA | | | UCUCGCCGCA |
| | | | | | | CTGAGGGGCA | | | CUGA |
| | | | | | | CTCCA | | | |
| | | | | | | CACCACGGGG | | | GGGGCACUCC |
| | | | | | | GCC | | | ACACCACGGG |
| | | | | | | [SEQ ID | | | GGCC |
| | | | | | | NO: | | | [SEQ ID |
| | | | | | | 1784] | | | NO: |
| | | | | | | | | | 1981] |
| miR- | 3842 | −2.27963 | −0.68707 | AACGTGAGAA | TTCGATCCAT | CATGCAGACT | AACGUGAGAA | UUCGAUCCAU | CAUGCAGACU |
| 1-1 | | | | GGATGGATCG | CCTTCTCAGA | GCCTGCTTGG | GGAUGGAUCG | CCUUCUCAGA | GCCUGCUUGG |
| | | | | TA | GTT | GTTCGATCCA | UA | GUU | GUUCGAUCCA |
| | | | | [SEQ ID | [SEQ ID | TCCTTCTCAG | [SEQ ID | [SEQ ID | UCCUUCUCAG |
| | | | | NO: | NO: | AGTTTATGGA | NO: | NO: | AGUUUAUGGA |
| | | | | 1625] | 1699] | CCTGCTAAGC | 1824] | 1894] | CCUGCUAAGC |
| | | | | | | TAAACGTGAG | | | UAAACGUGAG |
| | | | | | | AAGGATGGAT | | | AAGGAUGGAU |
| | | | | | | CGTACTCAGG | | | CGUACUCAGG |
| | | | | | | CCGGGACCTC | | | CCGGGACCUC |
| | | | | | | TCTCGCCGCA | | | UCUCGCCGCA |
| | | | | | | CTGAGGGGCA | | | CUGAGGGGCA |
| | | | | | | CTCCACACCA | | | CUCCACACCA |
| | | | | | | CGGGGGCC | | | CGGGGGCC |
| | | | | | | [SEQ ID | | | [SEQ ID |
| | | | | | | NO: | | | NO: |
| | | | | | | 1792] | | | 1989] |
| miR- | 2945 | −2.89804 | 0.152222 | TGTAGTAGAA | TGAGCCAAAG | CATGCAGACT | UGUAGUAGAA | UGAGCCAAAG | CAUGCAGACU |
| 1-1_M | | | | GGCTTTGGCT | CCTTCTACCG | GCCTGCTTGG | GGCUUUGGCU | CCUUCUACCG | GCCUGCUUGG |
| | | | | GA | ACA | GTGAGCCAAA | GA | ACA | GUGAGCCAAA |
| | | | | [SEQ ID | [SEQ ID | GCCTTCTACC | [SEQ ID | [SEQ ID | GCCUUCUACC |
| | | | | NO: | NO: | GACATATGGA | NO: | NO: | GACAUAUGGA |
| | | | | 685] | 1633] | CCTGCTAAGC | 1213] | 1828] | CCUGCUAAGC |
| | | | | | | TATGTAGTAG | (Same | | UAUGUAGUAG |
| | | | | | | AAGGCTTTGG | guide | | AAGGCUUUGG |
| | | | | | | CTGACTCAGG | as | | CUGACUCAGG |
| | | | | | | CCGGGACCTC | XD- | | CCGGGACCUC |
| | | | | | | TTCCGCCGCA | 14860) | | UUCCGCCGCA |
| | | | | | | CTGAGGGGCA | | | CUGAGGGGCA |
| | | | | | | CTCCACACCA | | | CUCCACACCA |
| | | | | | | CGGGGGCC | | | CGGGGGCC |
| | | | | | | [SEQ ID | | | [SEQ ID |
| | | | | | | NO: | | | NO: |
| | | | | | | 1711] | | | 1908] |
| miR- | 3341 | −2.70967 | 0.193529 | ATAGACATGA | GACTCAGCAT | CATGCAGACT | AUAGACAUGA | GACUCAGCAU | CAUGCAGACU |
| 1-1_M | | | | GGATGCTGAG | CCTCATGTGA | GCCTGCTTGG | GGAUGCUGAG | CCUCAUGUGA | GCCUGCUUGG |
| | | | | AC | TAT | GGACTCAGCA | AC | UAU | GGACUCAGCA |
| | | | | [SEQ ID | [SEQ ID | TCCTCATGTG | [SEQ ID | [SEQ ID | UCCUCAUGUG |
| | | | | NO: | NO: | ATATTATGGA | NO: | NO: | AUAUUAUGGA |
| | | | | 1617] | 1637] | CCTGCTAAGC | 1813] | 1832] | CCUGCUAAGC |
| | | | | | | TAATAGACAT | | | UAAUAGACAU |
| | | | | | | GAGGATGCTG | | | GAGGAUGCUG |
| | | | | | | AGACCTCAGG | | | AGACCUCAGG |
| | | | | | | CCGGGACCTC | | | CCGGGACCUC |
| | | | | | | TTCCGCCGCA | | | UUCCGCCGCA |
| | | | | | | CTGAGGGGCA | | | CUGAGGGGCA |
| | | | | | | CTCCA | | | CUCCACACCA |
| | | | | | | CACCACGGGG | | | CGGGGGCC |
| | | | | | | GCC | | | [SEQ ID |
| | | | | | | [SEQ ID | | | NO: |
| | | | | | | NO: | | | 1912] |
| | | | | | | 1715] | | | |
| miR- | 1755 | −2.62482 | 0.169485 | TCGGGTTGAA | CTCACTTCAG | CATGCAGACT | UCGGGUUGAA | CUCACUUCAG | CAUGCAGACU |
| 1-1_M | | | | ATCTGAAGTG | ATTTCAACGA | GCCTGCTTGG | AUCUGAAGUG | AUUUCAACGA | GCCUGCUUGG |
| | | | | TG | CGA | GCTCACTTCA | UG | CGA | GCUCACUUCA |
| | | | | [SEQ ID | [SEQ ID | GATTTCAACG | [SEQ ID | [SEQ ID | GAUUUCAACG |
| | | | | NO: | NO: | ACGATATGGA | NO: | NO: | ACGAUAUGGA |
| | | | | 657] | 1645] | CCTGCTAAGC | 1185] | 1840] | CCUGCUAAGC |
| | | | | | | TATCGGGTTG | | | UAUCGGGUUG |
| | | | | | | AAATCTGAAG | | | AAAUCUGAAG |
| | | | | | | TGTGCTCAGG | | | UGUGCUCAGG |
| | | | | | | CCGGGACCTC | | | CCGGGACCUC |
| | | | | | | TTCCGCCGCA | | | UUCCGCCGCA |
| | | | | | | CTGAGGGGCA | | | CUGAGGGGCA |
| | | | | | | CTCCACACCA | | | CUCCACACCA |
| | | | | | | CGGGGGCC | | | CGGGGGCC |
| | | | | | | [SEQ ID | | | [SEQ ID |
| | | | | | | NO: | | | NO: |
| | | | | | | 1723] | | | 1920] |
| miR- | 3302 | −2.57066 | 0.05742 | TTGAACAAGG | CGCAAATCAG | CATGCAGACT | UUGAACAAGG | CGCAAAUCAG | CAUGCAGACU |
| 1-1_M | | | | GGCTGATTTG | CCCCTTGTCG | GCCTGCTTGG | GGCUGAUUUG | CCCCUUGUCG | GCCUGCUUGG |
| | | | | GG | CAA | GCGCAAATCA | GG | CAA | GCGCAAAUCA |
| | | | | [SEQ ID | [SEQ ID | GCCCCTTGTC | [SEQ ID | [SEQ ID | GCCCCUUGUC |
| | | | | NO: | NO: | GCAATATGGA | NO: | NO: | GCAAUAUGGA |
| | | | | 688] | 1647] | CCTGCTAAGC | 1216] | 1842] | CCUGCUAAGC |
| | | | | | | TATTGAACAA | | | UAUUGAACAA |
| | | | | | | GGGGCTGATT | | | GGGGCUGAUU |
| | | | | | | TGGGCTCAGG | | | UGGGCUCAGG |
| | | | | | | CCGGGACCTC | | | CCGGGACCUC |
| | | | | | | TTCCGCCGCA | | | UUCCGCCGCA |
| | | | | | | CTGAGGGGCA | | | CUGAGGGGCA |
| | | | | | | CTCCACACCA | | | CUCCACACCA |
| | | | | | | CGGGGGCC | | | CGGGGGCC |
| | | | | | | [SEQ ID | | | [SEQ ID |
| | | | | | | NO: | | | NO: |
| | | | | | | 1727] | | | 1924] |
| miR- | 3330 | −2.47751 | 0.392579 | TATGCTGAGA | CGACATTATC | CATGCAGACT | UAUGCUGAGA | CGACAUUAUC | CAUGCAGACU |
| 1-1_M | | | | CTGATAATGT | AGTCTCAGGA | GCCTGCTTGG | CUGAUAAUGU | AGUCUCAGGA | GCCUGCUUGG |
| | | | | GG | ATA | GCGACATTAT | GG | AUA | GCGACAUUAU |
| | | | | [SEQ ID | [SEQ ID | CAGTCTCAGG | [SEQ ID | [SEQ ID | CAGUCUCAGG |
| | | | | NO: | NO: | AATATATGGA | NO: | NO: | AAUAUAUGGA |
| | | | | 1614] | 1659] | CCTGCTAAGC | 1811] | 1854] | CCUGCUAAGC |
| | | | | | | TATATGCTGA | | | UAUAUGCUGA |
| | | | | | | GACTGATAAT | | | GACUGAUAAU |
| | | | | | | GTGGCTCAGG | | | GUGGCUCAGG |
| | | | | | | CCGGGACCTC | | | CCGGGACCUC |
| | | | | | | TTCCGCCGCA | | | UUCCGCCGCA |
| | | | | | | CTGAGGGGCA | | | CUGAGGGGCA |
| | | | | | | CTC | | | CUCCACACCA |
| | | | | | | CACACCACGG | | | CGGGGGCC |
| | | | | | | GGGCC | | | [SEQ ID |
| | | | | | | [SEQ ID | | | NO: |
| | | | | | | NO: | | | 1940] |
| | | | | | | 1743] | | | |
| miR- | 2586 | −2.46317 | 0.179187 | TAGATTCAGA | CGAAGTTCTA | CATGCAGACT | UAGAUUCAGA | CGAAGUUCUA | CAUGCAGACU |
| 1-1M | | | | AGTAGAACTT | CTTCTGAACG | GCCTGCTTGG | AGUAGAACUU | CUUCUGAACG | GCCUGCUUGG |
| | | | | GG | CTA | GCGAAGTTCT | GG | CUA | GCGAAGUUCU |
| | | | | [SEQ ID | [SEQ ID | ACTTCTGAAC | [SEQ ID | [SEQ ID | ACUUCUGAAC |
| | | | | NO: | NO: | GCTATATGGA | NO: | NO: | GCUAUAUGGA |
| | | | | 1621] | 1662] | CCTGCTAAGC | 1816] | 1857] | CCUGCUAAGC |
| | | | | | | TATAGATTCA | | | UAUAGAUUCA |
| | | | | | | GAAGTAGAAC | | | GAAGUAGAAC |
| | | | | | | TTGGCTCAGG | | | UUGGCUCAGG |
| | | | | | | CCGGGACCTC | | | CCGGGACCUC |
| | | | | | | TTCCGCCGCA | | | UUCCGCCGCA |
| | | | | | | CTGAGGGGCA | | | CUGAGGGGCA |
| | | | | | | CTCCACACCA | | | CUCCACACCA |
| | | | | | | CGGGGGCC | | | CGGGGGCC |
| | | | | | | [SEQ ID | | | [SEQ ID |
| | | | | | | NO: | | | NO: |
| | | | | | | 1747] | | | 1944] |
| miR- | 3272 | −2.45517 | 0.050153 | AGGACTGTAG | GGAATATGTT | CATGCAGACT | AGGACUGUAG | GGAAUAUGUU | CAUGCAGACU |
| 1-1_M | | | | GCAACATATT | GCCTACAGCG | GCCTGCTTGG | GCAACAUAUU | GCCUACAGCG | GCCUGCUUGG |
| | | | | GC | CCT | GGGAATATGT | GC | CCU | GGGAAUAUGU |
| | | | | [SEQ ID | [SEQ ID | TGCCTACAGC | [SEQ ID | [SEQ ID | UGCCUACAGC |
| | | | | NO: | NO: | GCCTTATGGA | NO: | NO: | GCCUUAUGGA |
| | | | | 1618] | 1663] | CCTGCTAAGC | 1814] | 1858] | CCUGCUAAGC |
| | | | | | | TAAGGACTGT | | | UAAGGACUGU |
| | | | | | | AGGCAACATA | | | AGGCAACAUA |
| | | | | | | TTGCCTCAGG | | | UUGCCUCAGG |
| | | | | | | CCGGGACCTC | | | CCGGGACCUC |
| | | | | | | TTCCGCCGCA | | | UUCCGCCGCA |
| | | | | | | CTGAGGGGCA | | | CUGAGGGGCA |
| | | | | | | CTCCACACCA | | | CUCCACACCA |
| | | | | | | CGGGGGCC | | | CGGGGGCC |
| | | | | | | [SEQ ID | | | [SEQ ID |
| | | | | | | NO: | | | NO: |
| | | | | | | 1748] | | | 1945] |
| miR- | 2943 | −2.45216 | 0.338577 | TAGTAGAAGG | TGTCAGCCAA | CATGCAGACT | UAGUAGAAGG | UGUCAGCCAA | CAUGCAGACU |
| 1-1_M | | | | CTTTGGCTGA | AGCCTTCTCC | GCCTGCTTGG | CUUUGGCUGA | AGCCUUCUCC | GCCUGCUUGG |
| | | | | GA | CTA | GTGTCAGCCA | GA | CUA | GUGUCAGCCA |
| | | | | [SEQ ID | [SEQ ID | AAGCCTTCTC | [SEQ ID | [SEQ ID | AAGCCUUCUC |
| | | | | NO: | NO: | CCTATATGGA | NO: | NO: | CCUAUAUGGA |
| | | | | 683] | 1650] | CCTGCTAAGC | 1211] | 1845] | CCUGCUAAGC |
| | | | | | | TATAGTAGAA | | | UAUAGUAGAA |
| | | | | | | GGCTTTGGCT | | | GGCUUUGGCU |
| | | | | | | GAGACTCAGG | | | GAGACUCAGG |
| | | | | | | CCGGGACCTC | | | CCGGGACCUC |
| | | | | | | TTCCGCCGCA | | | UUCCGCCGCA |
| | | | | | | CTGAGGGGCA | | | CUGAGGGGCA |
| | | | | | | CTCC | | | CUCCACACCA |
| | | | | | | ACACCACGGG | | | CGGGGGCC |
| | | | | | | GGCC | | | [SEQ ID |
| | | | | | | [SEQ ID | | | NO: |
| | | | | | | NO: | | | 1946] |
| | | | | | | 1749] | | | |
| miR- | 3301 | −2.38494 | 0.327128 | TGAACAAGGG | TGCCAAATCA | CATGCAGACT | UGAACAAGGG | UGCCAAAUCA | CAUGCAGACU |
| 1-1_M | | | | GCTGATTTGG | GCCCCTTGCG | GCCTGCTTGG | GCUGAUUUGG | GCCCCUUGCG | GCCUGCUUGG |
| | | | | GA | TCA | GTGCCAAATC | GA | UCA | GUGCCAAAUC |
| | | | | [SEQ ID | [SEQ ID | AGCCCCTTGC | [SEQ ID | [SEQ ID | AGCCCCUUGC |
| | | | | NO: | NO: | GTCATATGGA | NO: | NO: | GUCAUAUGGA |
| | | | | 687] | 1673] | CCTGCTAAGC | 1215] | 1868] | CCUGCUAAGC |
| | | | | | | TATGAACAAG | | | UAUGAACAAG |
| | | | | | | GGGCTGATTT | | | GGGCUGAUUU |
| | | | | | | GGGACTCAGG | | | GGGACUCAGG |
| | | | | | | CCGGGACCTC | | | CCGGGACCUC |
| | | | | | | TTCCGCCGCA | | | UUCCGCCGCA |
| | | | | | | CTGAGGGGCA | | | CUGAGGGGCA |
| | | | | | | CTCCACACCA | | | CUCCACACCA |
| | | | | | | CGGGGGcc | | | CGGGGGCC |
| | | | | | | [SEQ ID | | | [SEQ ID |
| | | | | | | NO: | | | NO: |
| | | | | | | 1770] | | | 1967] |
| miR- | 2602 | −2.37919 | 0.040602 | TTTAGTAGTT | ATTCTATGGA | CATGCAGACT | UUUAGUAGUU | AUUCUAUGGA | CAUGCAGACU |
| 1-1_M | | | | GATCCATAGA | TCAACTACCG | GCCTGCTTGG | GAUCCAUAGA | UCAACUACCG | GCCUGCUUGG |
| | | | | TT | AAA | GATTCTATGG | UU | AAA | GAUUCUAUGG |
| | | | | [SEQ ID | [SEQ ID | ATCAACTACC | [SEQ ID | [SEQ ID | AUCAACUACC |
| | | | | NO: | NO: | GAAATATGGA | NO: | NO: | GAAAUAUGGA |
| | | | | 1616] | 1681] | CCTGCTAAGC | 202] | 1876] | CCUGCUAAGC |
| | | | | | | TATTTAGTAG | (Same | | UAUUUAGUAG |
| | | | | | | TTGATCCATA | guide | | UUGAUCCAUA |
| | | | | | | GATTCTCAGG | as | | GAUUCUCAGG |
| | | | | | | CCGGGACCTC | XD- | | CCGGGACCUC |
| | | | | | | TTCCGCCGCA | 14837) | | UUCCGCCGCA |
| | | | | | | CTGAGGGGCA | | | CUGAGGGGCA |
| | | | | | | CTCCACACCA | | | CUCCACACCA |
| | | | | | | CGGGGGCC | | | CGGGGGCC |
| | | | | | | [SEQ ID | | | [SEQ ID |
| | | | | | | NO: | | | NO: |
| | | | | | | 1772] | | | 1969] |
| miR- | 3302 | −2.68521 | −0.3818 | TTGAACAAGG | CCCAAACCAG | CCCAAAAGAG | UUGAACAAGG | CCCAAACCAG | CCCAAAAGAG |
| 100 | | | | GGCTGATTTG | ACCCTTGCTC | AGAAGATATT | GGCUGAUUUG | ACCCUUGCUC | AGAAGAUAUU |
| | | | | GG | AT | GAGGCCTGTT | GG | AU | GAGGCCUGUU |
| | | | | [SEQ ID | [SEQ ID | GCCACATTGA | [SEQ ID | [SEQ ID | GCCACAUUGA |
| | | | | NO: | NO: | ACAAGGGGCT | NO: | NO: | ACAAGGGGCU |
| | | | | 688] | 1638] | GATTTGGGGT | 1216] | 1833] | GAUUUGGGGU |
| | | | | | | ATTAGTCCGC | | | AUUAGUCCGC |
| | | | | | | CCAAACCAGA | | | CCAAACCAGA |
| | | | | | | CCCTTGCTCA | | | CCCUUGCUCA |
| | | | | | | TTGTGTCTGT | | | UUGUGUCUGU |
| | | | | | | TAGGCAATCT | | | UAGGCAAUCU |
| | | | | | | CACGGACCTG | | | CACGGACCUG |
| | | | | | | GGGC | | | GGGCUUUGCU |
| | | | | | | TTTGCTTATA | | | UAUAUGCC |
| | | | | | | TGCC | | | [SEQ ID |
| | | | | | | [SEQ ID | | | NO: |
| | | | | | | NO: | | | 1913] |
| | | | | | | 1716] | | | |
| miR- | 3043 | −2.67985 | −0.0584 | TTTGGTGCAA | AGCCTGCTTG | CCCAAAAGAG | UUUGGUGCAA | AGCCUGCUUG | CCCAAAAGAG |
| 100 | | | | AACAAACAGG | GTTTGCAACA | AGAAGATATT | AACAAACAGG | GUUUGCAACA | AGAAGAUAUU |
| | | | | CT | AT | GAGGCCTGTT | CU | AU | GAGGCCUGUU |
| | | | | [SEQ ID | [SEQ ID | GCCACATTTG | [SEQ ID | [SEQ ID | GCCACAUUUG |
| | | | | NO: | NO: | GTGCAAAACA | NO: | NO: | GUGCAAAACA |
| | | | | 1615] | 1639] | AACAGGCTGT | 1812] | 1834] | AACAGGCUGU |
| | | | | | | ATTAGTCCGA | | | AUUAGUCCGA |
| | | | | | | GCCTGCTTGG | | | GCCUGCUUGG |
| | | | | | | TTTGCAACAA | | | UUUGCAACAA |
| | | | | | | TTGTGTCTGT | | | UUGUGUCUGU |
| | | | | | | TAGGCAATCT | | | UAGGCAAUCU |
| | | | | | | CACGGACCTG | | | CACGGACCUG |
| | | | | | | GGGCTTTGCT | | | GGGCUUUGCU |
| | | | | | | TATATGCC | | | UAUAUGCC |
| | | | | | | [SEQ ID | | | [SEQ ID |
| | | | | | | NO: | | | NO: |
| | | | | | | 1717] | | | 1914] |
| miR- | 1755 | −2.66915 | −0.04161 | TCGGGTTGAA | CACACTCCAG | CCCAAAAGAG | UCGGGUUGAA | CACACUCCAG | CCCAAAAGAG |
| 100 | | | | ATCTGAAGTG | CTTTCAAACC | AGAAGATATT | AUCUGAAGUG | CUUUCAAACC | AGAAGAUAUU |
| | | | | TG | GT | GAGGCCTGTT | UG | GU | GAGGCCUGUU |
| | | | | [SEQ ID | [SEQ ID | GCCACATCGG | [SEQ ID | [SEQ ID | GCCACAUCGG |
| | | | | NO: | NO: | GTTGAAATCT | NO: | NO: | GUUGAAAUCU |
| | | | | 657] | 1640] | GAAGTGTGGT | 1185] | 1835] | GAAGUGUGGU |
| | | | | | | ATTAGTCCGC | | | AUUAGUCCGC |
| | | | | | | ACACTCCAGC | | | ACACUCCAGC |
| | | | | | | TTTCAAACCG | | | UUUCAAACCG |
| | | | | | | TTGTGTCTGT | | | UUGUGUCUGU |
| | | | | | | TAGGCAATCT | | | UAGGCAAUCU |
| | | | | | | CACGGACCTG | | | CACGGACCUG |
| | | | | | | GGGCTTTGCT | | | GGGCUUUGCU |
| | | | | | | TATATGCC | | | UAUAUGCC |
| | | | | | | [SEQ ID | | | [SEQ ID |
| | | | | | | NO: | | | NO: |
| | | | | | | 1718] | | | 1915] |
| miR- | 3272 | −2.65898 | −0.1606 | AGGACTGTAG | GCAATACGTT | CCCAAAAGAG | AGGACUGUAG | GCAAUACGUU | CCCAAAAGAG |
| 100 | | | | GCAACATATT | TCCTACAATC | AGAAGATATT | GCAACAUAUU | UCCUACAAUC | AGAAGAUAUU |
| | | | | GC | CA | GAGGCCTGTT | GC | CA | GAGGCCUGUU |
| | | | | [SEQ ID | [SEQ ID | GCCACAAGGA | [SEQ ID | [SEQ ID | GCCACAAGGA |
| | | | | NO: | NO: | CTGTAGGCAA | NO: | NO: | CUGUAGGCAA |
| | | | | 1618] | 1641] | CATATTGCGT | 1814] | 1836] | CAUAUUGCGU |
| | | | | | | ATTAGTCCGG | | | AUUAGUCCGG |
| | | | | | | CAATACGTTT | | | CAAUACGUUU |
| | | | | | | CCTACAATCC | | | CCUACAAUCC |
| | | | | | | ATGTGTCTGT | | | AUGUGUCUGU |
| | | | | | | TAGGCAATCT | | | UAGGCAAUCU |
| | | | | | | CACGGACCTG | | | CACGGACCUG |
| | | | | | | GGGC | | | GGGCUUUGCU |
| | | | | | | TTTGCTTATA | | | UAUAUGCC |
| | | | | | | TGCC | | | [SEQ ID |
| | | | | | | [SEQ ID | | | NO: |
| | | | | | | NO: | | | 1916] |
| | | | | | | 1719] | | | |
| miR- | 1578 | −2.43871 | −0.43493 | TGGAATTTCT | ACAGCAATTC | CCCAAAAGAG | UGGAAUUUCU | ACAGCAAUUC | CCCAAAAGAG |
| 100 | | | | CTGAACTGCT | CGAGAAACTC | AGAAGATATT | CUGAACUGCU | CGAGAAACUC | AGAAGAUAUU |
| | | | | GT | CT | GAGGCCTGTT | GU | CU | GAGGCCUGUU |
| | | | | [SEQ ID | [SEQ ID | GCCACATGGA | [SEQ ID | [SEQ ID | GCCACAUGGA |
| | | | | NO: | NO: | ATTTCTCTGA | NO: | NO: | AUUUCUCUGA |
| | | | | 1626] | 1665] | ACTGCTGTGT | 1820] | 1860] | ACUGCUGUGU |
| | | | | | | ATTAGTCCGA | | | AUUAGUCCGA |
| | | | | | | CAGCAATTCC | | | CAGCAAUUCC |
| | | | | | | GAGAAACTCC | | | GAGAAACUCC |
| | | | | | | TTGTGTCTGT | | | UUGUGUCUGU |
| | | | | | | TAGGCAATCT | | | UAGGCAAUCU |
| | | | | | | CACGGACCTG | | | CACGGACCUG |
| | | | | | | GGGCTTTGCT | | | GGGCUUUGCU |
| | | | | | | TATATGCC | | | UAUAUGCC |
| | | | | | | [SEQ ID | | | [SEQ ID |
| | | | | | | NO: | | | NO: |
| | | | | | | 1751] | | | 1948] |
| miR- | 2945 | −2.38747 | −0.10088 | TGTAGTAGAA | TCAGCCCAAG | CCCAAAAGAG | UGUAGUAGAA | UCAGCCCAAG | CCCAAAAGAG |
| 100 | | | | GGCTTTGGCT | ACTTCTAATA | AGAAGATATT | GGCUUUGGCU | ACUUCUAAUA | AGAAGAUAUU |
| | | | | GA | CT | GAGGCCTGTT | GA | CU | GAGGCCUGUU |
| | | | | [SEQ ID | [SEQ ID | GCCACATGTA | [SEQ ID | [SEQ ID | GCCACAUGUA |
| | | | | NO: | NO: | GTAGAAGGCT | NO: | NO: | GUAGAAGGCU |
| | | | | 685] | 1678] | TTGGCTGAGT | 1213] | 1873] | UUGGCUGAGU |
| | | | | | | ATTAGTCCGT | (Same | | AUUAGUCCGU |
| | | | | | | CAGCCCAAGA | guide | | CAGCCCAAGA |
| | | | | | | CTTCTAATAC | as | | CUUCUAAUAC |
| | | | | | | TTGTGTCTGT | XD- | | UUGUGUCUGU |
| | | | | | | TAGGCAATCT | 14860) | | UAGGCAAUCU |
| | | | | | | CACGGACCTG | | | CACGGACCUG |
| | | | | | | GGGCTTTGCT | | | GGGCUUUGCU |
| | | | | | | TATATGCC | | | UAUAUGCC |
| | | | | | | [SEQ ID | | | [SEQ ID |
| | | | | | | NO: | | | NO: |
| | | | | | | 1768] | | | 1965] |
| miR- | 2586 | −2.32164 | −0.18294 | TAGATTCAGA | CCAAGTCCTA | CCCAAAAGAG | UAGAUUCAGA | CCAAGUCCUA | CCCAAAAGAG |
| 100 | | | | AGTAGAACTT | ATTCTGACTC | AGAAGATATT | AGUAGAACUU | AUUCUGACUC | AGAAGAUAUU |
| | | | | GG | TT | GAGGCCTGTT | GG | UU | GAGGCCUGUU |
| | | | | [SEQ ID | [SEQ ID | GCCACATAGA | [SEQ ID | [SEQ ID | GCCACAUAGA |
| | | | | NO: | NO: | TTCAGAAGTA | NO: | NO: | UUCAGAAGUA |
| | | | | 1621] | 1692] | GAACTTGGGT | 1816] | 1887] | GAACUUGGGU |
| | | | | | | ATTAGTCCGC | | | AUUAGUCCGC |
| | | | | | | CAAGTCCTAA | | | CAAGUCCUAA |
| | | | | | | TTCTGACTCT | | | UUCUGACUCU |
| | | | | | | TTGTGTCTGT | | | UUGUGUCUGU |
| | | | | | | TAGGCAATCT | | | UAGGCAAUCU |
| | | | | | | CACGGACCTG | | | CACGGACCUG |
| | | | | | | GGGCTTTGCT | | | GGGCUUUGCU |
| | | | | | | TATATGCC | | | UAUAUGCC |
| | | | | | | [SEQ ID | | | [SEQ ID |
| | | | | | | NO: | | | NO: |
| | | | | | | 1785] | | | 1982] |
| miR- | 3341 | −2.20897 | 0.022563 | ATAGACATGA | GTCTCAACAT | CCCAAAAGAG | AUAGACAUGA | GUCUCAACAU | CCCAAAAGAG |
| 100 | | | | GGATGCTGAG | ACTCATGCCT | AGAAGATATT | GGAUGCUGAG | ACUCAUGCCU | AGAAGAUAUU |
| | | | | AC | AA | GAGGCCTGTT | AC | AA | GAGGCCUGUU |
| | | | | [SEQ ID | [SEQ ID | GCCACAATAG | [SEQ ID | [SEQ ID | GCCACAAUAG |
| | | | | NO: | NO: | ACATGAGGAT | NO: | NO: | ACAUGAGGAU |
| | | | | 1617] | 2008] | GCTGAGACGT | 1813] | 2010] | GCUGAGACGU |
| | | | | | | ATTAGTCCGG | | | AUUAGUCCGG |
| | | | | | | TCTCAACATA | | | UCUCAACAUA |
| | | | | | | CTCATGCCTA | | | CUCAUGCCUA |
| | | | | | | ATGTGTCTGT | | | AUGUGUCUGU |
| | | | | | | TAGGCAATCT | | | UAGGCAAUCU |
| | | | | | | CACGGACCTG | | | CACGGACCUG |
| | | | | | | GGGCTTTGCT | | | GGGCUUUGCU |
| | | | | | | TATATGCC | | | UAUAUGCC |
| | | | | | | [SEQ ID | | | [SEQ ID |
| | | | | | | NO: | | | NO: |
| | | | | | | 2009] | | | 2011] |
| miR- | 893 | −2.15837 | −0.34705 | TTTGTTACTG | CAGAGGCCGA | CCCAAAAGAG | UUUGUUACUG | CAGAGGCCGA | CCCAAAAGAG |
| 100 | | | | TTTCGACCTC | CACAGTACCA | AGAAGATATT | UUUCGACCUC | CACAGUACCA | AGAAGAUAUU |
| | | | | TG | AT | GAGGCCTGTT | UG | AU | GAGGCCUGUU |
| | | | | [SEQ ID | [SEQ ID | GCCACATTTG | [SEQ ID | [SEQ ID | GCCACAUUUG |
| | | | | NO: | NO: | TTACTGTTTC | NO: | NO: | UUACUGUUUC |
| | | | | 2012] | 2013] | GACCTCTGGT | 2015] | 2016] | GACCUCUGGU |
| | | | | | | ATTAGTCCGC | | | AUUAGUCCGC |
| | | | | | | AGAGGCCGAC | | | AGAGGCCGAC |
| | | | | | | ACAGTACCAA | | | ACAGUACCAA |
| | | | | | | TTGTGTCTGT | | | UUGUGUCUGU |
| | | | | | | TAGGCAATCT | | | UAGGCAAUCU |
| | | | | | | CACGGACCTG | | | CACGGACCUG |
| | | | | | | GGGCTTTGCT | | | GGGCUUUGCU |
| | | | | | | TATATGCC | | | UAUAUGCC |
| | | | | | | [SEQ ID | | | [SEQ ID |
| | | | | | | NO: | | | NO: |
| | | | | | | 2014] | | | 2017] |
| miR- | 3330 | −2.15241 | 0.12385 | TATGCTGAGA | CCACATCATC | CCCAAAAGAG | UAUGCUGAGA | CCACAUCAUC | CCCAAAAGAG |
| 100 | | | | CTGATAATGT | CGTCTCAACA | AGAAGATATT | CUGAUAAUGU | CGUCUCAACA | AGAAGAUAUU |
| | | | | GG | TT | GAGGCCTGTT | GG | UU | GAGGCCUGUU |
| | | | | [SEQ ID | [SEQ ID | GCCACATATG | [SEQ ID | [SEQ ID | GCCACAUAUG |
| | | | | NO: | NO: | CTGAGACTGA | NO: | NO: | CUGAGACUGA |
| | | | | 1614] | 2018] | TAATGTGGGT | 1811] | 2020] | UAAUGUGGGU |
| | | | | | | ATTAGTCCGC | | | AUUAGUCCGC |
| | | | | | | CACATCATCC | | | CACAUCAUCC |
| | | | | | | GTCTCAACAT | | | GUCUCAACAU |
| | | | | | | TTGTGTCTGT | | | UUGUGUCUGU |
| | | | | | | TAGGCAATCT | | | UAGGCAAUCU |
| | | | | | | CACGGACCTG | | | CACGGACCUG |
| | | | | | | GGGCTTTGCT | | | GGGCUUUGCU |
| | | | | | | TATATGCC | | | UAUAUGCC |
| | | | | | | [SEQ ID | | | [SEQ ID |
| | | | | | | NO: | | | NO: |
| | | | | | | 2019] | | | 2021] |
| miR- | 3302 | −2.62238 | −0.19177 | TTGAACAAGG | CCCAAACCAG | CCCAAAAGAG | UUGAACAAGG | CCCAAACCAG | CCCAAAAGAG |
| 100M | | | | GGCTGATTTG | ACCCTTGCTC | AGAAGATATT | GGCUGAUUUG | ACCCUUGCUC | AGAAGAUAUU |
| | | | | GG | AT | GATGCCTGTT | GG | AU | GAUGCCUGUU |
| | | | | [SEQ ID | [SEQ ID | GCCACATTGA | [SEQ ID | [SEQ ID | GCCACAUUGA |
| | | | | NO: | NO: | ACAAGGGGCT | NO: | NO: | ACAAGGGGCU |
| | | | | 688] | 1638] | GATTTGGGGT | 1216] | 1833] | GAUUUGGGGU |
| | | | | | | ATTAGTCCGC | | | AUUAGUCCGC |
| | | | | | | CCAAACCAGA | | | CCAAACCAGA |
| | | | | | | CCCTTGCTCA | | | CCCUUGCUCA |
| | | | | | | TTGTGTCTGT | | | UUGUGUCUGU |
| | | | | | | TAGGCTATTC | | | UAGGCUAUUC |
| | | | | | | CACGGACCTG | | | CACGGACCUG |
| | | | | | | GGGCTTTGCT | | | GGGCUUUGCU |
| | | | | | | TATATGCC | | | UAUAUGCC |
| | | | | | | [SEQ ID | | | [SEQ ID |
| | | | | | | NO: | | | NO: |
| | | | | | | 1724] | | | 1921] |
| miR- | 3272 | −2.56927 | −0.01914 | AGGACTGTAG | GCAATACGTT | CCCAAAAGAG | AGGACUGUAG | GCAAUACGUU | CCCAAAAGAG |
| 100_M | | | | GCAACATATT | TCCTACAATC | AGAAGATATT | GCAACAUAUU | UCCUACAAUC | AGAAGAUAUU |
| | | | | GC | CA | GATGCCTGTT | GC | CA | GAUGCCUGUU |
| | | | | [SEQ ID | [SEQ ID | GCCACAAGGA | [SEQ ID | [SEQ ID | GCCACAAGGA |
| | | | | NO: | NO: | CTGTAGGCAA | NO: | NO: | CUGUAGGCAA |
| | | | | 1618] | 1641] | CATATTGCGT | 1814] | 1836] | CAUAUUGCGU |
| | | | | | | ATTAGTCCGG | | | AUUAGUCCGG |
| | | | | | | CAATACGTTT | | | CAAUACGUUU |
| | | | | | | CCTACAATCC | | | CCUACAAUCC |
| | | | | | | ATGTGTCTGT | | | AUGUGUCUGU |
| | | | | | | TAGGCTATTC | | | UAGGCUAUUC |
| | | | | | | CACGGACCTG | | | CACGGACCUG |
| | | | | | | GGGCTTTGCT | | | GGGCUUUGCU |
| | | | | | | TATATGCC | | | UAUAUGCC |
| | | | | | | [SEQ ID | | | [SEQ ID |
| | | | | | | NO: | | | NO: |
| | | | | | | 17291 | | | 1926] |
| miR- | 3133 | −2.49525 | 0.238806 | TATGTCTTGG | CAGTGACTCA | CCCAAAAGAG | UAUGUCUUGG | CAGUGACUCA | CCCAAAAGAG |
| 100_M | | | | CTTGATTCAC | CGCCAAGCCA | AGAAGATATT | CUUGAUUCAC | CGCCAAGCCA | AGAAGAUAUU |
| | | | | TG | TT | GATGCCTGTT | UG | UU | GAUGCCUGUU |
| | | | | [SEQ ID | [SEQ ID | GCCACATATG | [SEQ ID | [SEQ ID | GCCACAUAUG |
| | | | | NO: | NO: | TCTTGGCTTG | NO: | NO: | UCUUGGCUUG |
| | | | | 1624] | 1656] | ATTCACTGGT | 1819] | 1851] | AUUCACUGGU |
| | | | | | | ATTAGTCCGC | | | AUUAGUCCGC |
| | | | | | | AGTGACTCAC | | | AGUGACUCAC |
| | | | | | | GCCAAGCCAT | | | GCCAAGCCAU |
| | | | | | | TTGTGTCTGT | | | UUGUGUCUGU |
| | | | | | | TAGGCTATTC | | | UAGGCUAUUC |
| | | | | | | CACGGACCTG | | | CACGGACCUG |
| | | | | | | GGGCTTTGCT | | | GGGCUUUGCU |
| | | | | | | TATATGCC | | | UAUAUGCC |
| | | | | | | [SEQ ID | | | [SEQ ID |
| | | | | | | NO: | | | NO: |
| | | | | | | 1740] | | | 1937] |
| miR- | 1755 | −2.4723 | −0.07055 | TCGGGTTGAA | CACACTCCAG | CCCAAAAGAG | UCGGGUUGAA | CACACUCCAG | CCCAAAAGAG |
| 100_M | | | | ATCTGAAGTG | CTTTCAAACC | AGAAGATATT | AUCUGAAGUG | CUUUCAAACC | AGAAGAUAUU |
| | | | | TG | GT | GATGCCTGTT | UG | GU | GAUGCCUGUU |
| | | | | [SEQ ID | [SEQ ID | GCCACAT | [SEQ ID | [SEQ ID | GCCACAUC |
| | | | | NO: | NO: | CGGGTTGAAA | NO: | NO: | GGGUUGAAAU |
| | | | | 657] | 1640] | TCTGAAGTGT | 1185] | 1835] | CUGAAGUGUG |
| | | | | | | GGTATTAGTC | | | GUAUUAGUCC |
| | | | | | | CGCACACTCC | | | GCACACUCCA |
| | | | | | | AGCTTTCAAA | | | GCUUUCAAAC |
| | | | | | | CCGTTGTGTC | | | CGUUGUGUCU |
| | | | | | | TGTTAGGCTA | | | GUUAGGCUAU |
| | | | | | | TTCCACGGAC | | | UCCACGGACC |
| | | | | | | CTGGGGCTTT | | | UGGGGCUUUG |
| | | | | | | GCTTATATGC | | | CUUAUAUGCC |
| | | | | | | C | | | [SEQ ID |
| | | | | | | [SEQ ID | | | NO: |
| | | | | | | NO: | | | 1942] |
| | | | | | | 1745] | | | |
| miR- | 1231 | −2.37228 | −0.26919 | TTCACTTTAG | CTGCTACCAG | CCCAAAAGAG | UUCACUUUAG | CUGCUACCAG | CCCAAAAGAG |
| 100_M | | | | CACTGATAGC | GGCTAAAATG | AGAAGATATT | CACUGAUAGC | GGCUAAAAUG | AGAAGAUAUU |
| | | | | AG | AT | GATGCCTGTT | AG | AU | GAUGCCUGUU |
| | | | | [SEQ ID | [SEQ ID | GCCACATTCA | [SEQ ID | [SEQ ID | GCCACAUUCA |
| | | | | NO: | NO: | CTTTAGCACT | NO: | NO: | CUUUAGCACU |
| | | | | 1627] | 1682] | GATAGCAGGT | 1825] | 1877] | GAUAGCAGGU |
| | | | | | | ATTAGTCCGC | | | AUUAGUCCGC |
| | | | | | | TGCTACCAGG | | | UGCUACCAGG |
| | | | | | | GCTAAAATGA | | | GCUAAAAUGA |
| | | | | | | TTGTGTCTGT | | | UUGUGUCUGU |
| | | | | | | TAGGCTATTC | | | UAGGCUAUUC |
| | | | | | | CACGGACCTG | | | CACGGACCUG |
| | | | | | | GGGCTTTGCT | | | GGGCUUUGCU |
| | | | | | | TATATGCC | | | UAUAUGCC |
| | | | | | | [SEQ ID | | | [SEQ ID |
| | | | | | | NO: | | | NO: |
| | | | | | | 1773] | | | 1970] |
| miR- | 3043 | −2.35911 | 0.045827 | TTTGGTGCAA | AGCCTGCTTG | CCCAAAAGAG | UUUGGUGCAA | AGCCUGCUUG | CCCAAAAGAG |
| 100_M | | | | AACAAACAGG | GTTTGCAACA | AGAAGATATT | AACAAACAGG | GUUUGCAACA | AGAAGAUAUU |
| | | | | CT | AT | GATGCCTGTT | CU | AU | GAUGCCUGUU |
| | | | | [SEQ ID | [SEQ ID | GCCACATTTG | [SEQ ID | [SEQ ID | GCCACAUUUG |
| | | | | NO: | NO: | GTGCAAAACA | NO: | NO: | GUGCAAAACA |
| | | | | 1615] | 1639] | AACAGGCTGT | 1812] | 1834] | AACAGGCUGU |
| | | | | | | ATTAGTCCGA | | | AUUAGUCCGA |
| | | | | | | GCCTGCTTGG | | | GCCUGCUUGG |
| | | | | | | TTTGCAACAA | | | UUUGCAACAA |
| | | | | | | TTGTGTCTGT | | | UUGUGUCUGU |
| | | | | | | TAGGCTATTC | | | UAGGCUAUUC |
| | | | | | | CACGGACCTG | | | CACGGACCUG |
| | | | | | | GGGCTTTGCT | | | GGGCUUUGCU |
| | | | | | | TATATGCC | | | UAUAUGCC |
| | | | | | | [SEQ ID | | | [SEQ ID |
| | | | | | | NO: | | | NO: |
| | | | | | | 1776] | | | 1973] |
| miR- | 3301 | −2.30882 | −0.2279 | TGAACAAGGG | TCCCAACTCA | CCCAAAAGAG | UGAACAAGGG | UCCCAACUCA | CCCAAAAGAG |
| 100_M | | | | GCTGATTTGG | TCCCCTTATT | AGAAGATATT | GCUGAUUUGG | UCCCCUUAUU | AGAAGAUAUU |
| | | | | GA | CT | GATGCCTGTT | GA | CU | GAUGCCUGUU |
| | | | | [SEQ ID | [SEQ ID | GCCACATGAA | [SEQ ID | [SEQ ID | GCCACAUGAA |
| | | | | NO: | NO: | CAAGGGGCTG | NO: | NO: | CAAGGGGCUG |
| | | | | 687] | 1695] | ATTTGGGAGT | 1215] | 1890] | AUUUGGGAGU |
| | | | | | | ATTAGTCCGT | | | AUUAGUCCGU |
| | | | | | | CCCAACTCAT | | | CCCAACUCAU |
| | | | | | | CCCCTTA | | | CCCCUUAU |
| | | | | | | TTCTTGTGTC | | | UCUUGUGUCU |
| | | | | | | TGTTAGGCTA | | | GUUAGGCUAU |
| | | | | | | TTCCACGGAC | | | UCCACGGACC |
| | | | | | | CTGGGGCTTT | | | UGGGGCUUUG |
| | | | | | | GCTTATATGC | | | CUUAUAUGCC |
| | | | | | | C | | | [SEQ ID |
| | | | | | | [SEQ ID | | | NO: |
| | | | | | | NO: | | | 1985] |
| | | | | | | 1788] | | | |
| miR- | 2945 | −2.26187 | 0.044364 | TGTAGTAGAA | TCAGCCCAAG | CCCAAAAGAG | UGUAGUAGAA | UCAGCCCAAG | CCCAAAAGAG |
| 100_M | | | | GGCTTTGGCT | ACTTCTAATA | AGAAGATATT | GGCUUUGGCU | ACUUCUAAUA | AGAAGAUAUU |
| | | | | GA | CT | GATGCCTGTT | GA | CU | GAUGCCUGUU |
| | | | | [SEQ ID | [SEQ ID | GCCACATGTA | [SEQ ID | [SEQ ID | GCCACAUGUA |
| | | | | NO: | NO: | GTAGAAGGCT | NO: | NO: | GUAGAAGGCU |
| | | | | 685] | 1678] | TTGGCTGAGT | 1213] | 1873] | UUGGCUGAGU |
| | | | | | | ATTAGTCCGT | (Same | | AUUAGUCCGU |
| | | | | | | CAGCCCAAGA | guide | | CAGCCCAAGA |
| | | | | | | CTTCTAATAC | as | | CUUCUAAUAC |
| | | | | | | TTGTGTCTGT | XD- | | UUGUGUCUGU |
| | | | | | | TAGGCTATTC | 14860) | | UAGGCUAUUC |
| | | | | | | CACGGACCTG | | | CACGGACCUG |
| | | | | | | GGGCTTTGCT | | | GGGCUUUGCU |
| | | | | | | TATATGCC | | | UAUAUGCC |
| | | | | | | [SEQ ID | | | [SEQ ID |
| | | | | | | NO: | | | NO: |
| | | | | | | 1797] | | | 1994] |
| miR- | 1578 | −2.24051 | −0.33444 | TGGAATTTCT | ACAGCAATTC | CCCAAAAGAG | UGGAAUUUCU | ACAGCAAUUC | CCCAAAAGAG |
| 100_M | | | | CTGAACTGCT | CGAGAAACTC | AGAAGATATT | CUGAACUGCU | CGAGAAACUC | AGAAGAUAUU |
| | | | | GT | CT | GATGCCTGTT | GU | CU | GAUGCCUGUU |
| | | | | [SEQ ID | [SEQ ID | GCCACATGGA | [SEQ ID | [SEQ ID | GCCACAUGGA |
| | | | | NO: | NO: | ATTTCTCTGA | NO: | NO: | AUUUCUCUGA |
| | | | | 1626] | 1665] | ACTGCTGTGT | 1820] | 1860] | ACUGCUGUGU |
| | | | | | | ATTAGTCCGA | | | AUUAGUCCGA |
| | | | | | | CAGCAATTCC | | | CAGCAAUUCC |
| | | | | | | GAGAAACTCC | | | GAGAAACUCC |
| | | | | | | TTGTGTCTGT | | | UUGUGUCUGU |
| | | | | | | TAGGCTATTC | | | UAGGCUAUUC |
| | | | | | | CACGGACCTG | | | CACGGACCUG |
| | | | | | | GGGCTTTGCT | | | GGGCUUUGCU |
| | | | | | | TATATGCC | | | UAUAUGCC |
| | | | | | | [SEQ ID | | | [SEQ ID |
| | | | | | | NO: | | | NO: |
| | | | | | | 1808] | | | 2005] |
| miR- | 1580 | −2.12895 | −0.08237 | ACTGGAATTT | AGCAGTCCAG | CCCAAAAGAG | ACUGGAAUUU | AGCAGUCCAG | CCCAAAAGAG |
| 100_M | | | | CTCTGAACTG | CGAAATTACA | AGAAGATATT | CUCUGAACUG | CGAAAUUACA | AGAAGAUAUU |
| | | | | CT | GA | GATGCCTGTT | CU | GA | GAUGCCUGUU |
| | | | | [SEQ ID | [SEQ ID | GCCACAACTG | [SEQ ID | [SEQ ID | GCCACAACUG |
| | | | | NO: | NO: | GAATTTCTCT | NO: | NO: | GAAUUUCUCU |
| | | | | 1622] | 2022] | GAACTGCTGT | 1817] | 2024] | GAACUGCUGU |
| | | | | | | ATTAGTCCGA | | | AUUAGUCCGA |
| | | | | | | GCAGTCCAGC | | | GCAGUCCAGC |
| | | | | | | GAAATTACAG | | | GAAAUUACAG |
| | | | | | | ATGTGTCTGT | | | AUGUGUCUGU |
| | | | | | | TAGGCTATTC | | | UAGGCUAUUC |
| | | | | | | CACGGACCTG | | | CACGGACCUG |
| | | | | | | GGGCTTTGCT | | | GGGCUUUGCU |
| | | | | | | TATATGCC | | | UAUAUGCC |
| | | | | | | [SEQ ID | | | [SEQ ID |
| | | | | | | NO: | | | NO: |
| | | | | | | 2023] | | | 2025] |
| miR- | 3332 | −2.50104 | −0.209 | AGGATGCTGA | ACATTATCAG | GGCTACAGAG | AGGAUGCUGA | ACAUUAUCAG | GGCUACAGAG |
| 122 | | | | GACTGATAAT | TATCAGCATA | TTTCCTTAGC | GACUGAUAAU | UAUCAGCAUA | UUUCCUUAGC |
| | | | | GT | AT | AGAGCTGAGG | GU | AU | AGAGCUGAGG |
| | | | | [SEQ ID | [SEQ ID | ATGCTGAGAC | [SEQ ID | [SEQ ID | AUGCUGAGAC |
| | | | | NO: | NO: | TGATAATGTT | NO: | NO: | UGAUAAUGUU |
| | | | | 1623] | 1655] | GTCTAAACTA | 1818] | 1850] | GUCUAAACUA |
| | | | | | | TACATTATCA | | | UACAUUAUCA |
| | | | | | | GTATCAGCAT | | | GUAUCAGCAU |
| | | | | | | AATTAGCTAC | | | AAUUAGCUAC |
| | | | | | | TGCTAGGCAA | | | UGCUAGGCAA |
| | | | | | | TCCTTCCCTC | | | UCCUUCCCUC |
| | | | | | | GATAAATGTC | | | GAUAAAUGUC |
| | | | | | | TTGGCATCGT | | | UUGGCAUCGU |
| | | | | | | TTGCTTTG | | | UUGCUUUG |
| | | | | | | [SEQ ID | | | [SEQ ID |
| | | | | | | NO: | | | NO: |
| | | | | | | 1739] | | | 1936] |
| miR- | 3341 | −2.4857 | 0.187025 | ATAGACATGA | GTCTCAGCAT | GGCTACAGAG | AUAGACAUGA | GUCUCAGCAU | GGCUACAGAG |
| 122 | | | | GGATGCTGAG | CATCATGTCG | TTTCCTTAGC | GGAUGCUGAG | CAUCAUGUCG | UUUCCUUAGC |
| | | | | AC | CT | AGAGCTGATA | AC | CU | AGAGCUGAUA |
| | | | | [SEQ ID | [SEQ ID | GACATGAGGA | [SEQ ID | [SEQ ID | GACAUGAGGA |
| | | | | NO: | NO: | TGCTGAGACT | NO: | NO: | UGCUGAGACU |
| | | | | 1617] | 1657] | GTCTAAACTA | 1813] | 1852] | GUCUAAACUA |
| | | | | | | TGTCTCAGCA | | | UGUCUCAGCA |
| | | | | | | TCATCATGTC | | | UCAUCAUGUC |
| | | | | | | GCTTAGCTAC | | | GCUUAGCUAC |
| | | | | | | TGCTAGGCAA | | | UGCUAGGCAA |
| | | | | | | TCCTTCCCTC | | | UCCUUCCCUC |
| | | | | | | GATAAATGTC | | | GAUAAAUGUC |
| | | | | | | TTGGCATCGT | | | UUGGCAUCGU |
| | | | | | | TTGCTTTG | | | UUGCUUUG |
| | | | | | | [SEQ ID | | | [SEQ ID |
| | | | | | | NO: | | | NO: |
| | | | | | | 1741] | | | 1938] |
| miR- | 1755 | −2.42198 | 0.034447 | TCGGGTTGAA | CACACTTCAG | GGCTACAGAG | UCGGGUUGAA | CACACUUCAG | GGCUACAGAG |
| 122 | | | | ATCTGAAGTG | ACTTCAACCA | TTTCCTTAGC | AUCUGAAGUG | ACUUCAACCA | UUUCCUUAGC |
| | | | | TG | TA | AGAGCTGTCG | UG | UA | AGAGCUGUCG |
| | | | | [SEQ ID | [SEQ ID | GGTTGAAATC | [SEQ ID | [SEQ ID | GGUUGAAAUC |
| | | | | NO: | NO: | TGAAGTGTGT | NO: | NO: | UGAAGUGUGU |
| | | | | 657] | 1646] | GTCTAAACTA | 1185] | 1841] | GUCUAAACUA |
| | | | | | | TCACACTTCA | | | UCACACUUCA |
| | | | | | | GACTTCAACC | | | GACUUCAACC |
| | | | | | | ATATAGCTAC | | | AUAUAGCUAC |
| | | | | | | TGCTAGGCAA | | | UGCUAGGCAA |
| | | | | | | TCCTTCCCTC | | | UCCUUCCCUC |
| | | | | | | GATAAATGTC | | | GAUAAAUGUC |
| | | | | | | TTGGCATCGT | | | UUGGCAUCGU |
| | | | | | | TTGCTTTG | | | UUGCUUUG |
| | | | | | | [SEQ ID | | | [SEQ ID |
| | | | | | | NO: | | | NO: |
| | | | | | | 1760] | | | 1957] |
| miR- | 2586 | −2.35707 | 0.153236 | TAGATTCAGA | CCAAGTTCTA | GGCTACAGAG | UAGAUUCAGA | CCAAGUUCUA | GGCUACAGAG |
| 122 | | | | AGTAGAACTT | CCTCTGAATA | TTTCCTTAGC | AGUAGAACUU | CCUCUGAAUA | UUUCCUUAGC |
| | | | | GG | GA | AGAGCTGTAG | GG | GA | AGAGCUGUAG |
| | | | | [SEQ ID | [SEQ ID | ATTCAGAAGT | [SEQ ID | [SEQ ID | AUUCAGAAGU |
| | | | | NO: | NO: | AGAACTTGGT | NO: | NO: | AGAACUUGGU |
| | | | | 1621] | 1685] | GTCTAAACTA | 1816] | 1880] | GUCUAAACUA |
| | | | | | | TCCAAGTTCT | | | UCCAAGUUCU |
| | | | | | | ACCTCTGAAT | | | ACCUCUGAAU |
| | | | | | | AGATAGCTAC | | | AGAUAGCUAC |
| | | | | | | TGCTAGGCAA | | | UGCUAGGCAA |
| | | | | | | TCCTTCCCTC | | | UCCUUCCCUC |
| | | | | | | GATAAATGTC | | | GAUAAAUGUC |
| | | | | | | TTGGCATCGT | | | UUGGCAUCGU |
| | | | | | | TTGCTTTG | | | UUGCUUUG |
| | | | | | | [SEQ ID | | | [SEQ ID |
| | | | | | | NO: | | | NO: |
| | | | | | | 1777] | | | 1974] |
| miR- | 3273 | −2.30907 | −0.11515 | TAGGACTGTA | CAATATGTTG | GGCTACAGAG | UAGGACUGUA | CAAUAUGUUG | GGCUACAGAG |
| 122 | | | | GGCAACATAT | CATACAGTCA | TTTCCTTAGC | GGCAACAUAU | CAUACAGUCA | UUUCCUUAGC |
| | | | | TG | GA | AGAGCTGTAG | UG | GA | AGAGCUGUAG |
| | | | | [SEQ ID | [SEQ ID | GACTGTAGGC | [SEQ ID | [SEQ ID | GACUGUAGGC |
| | | | | NO: | NO: | AACATATTGT | NO: | NO: | AACAUAUUGU |
| | | | | 1628] | 1694] | GTCTAAACTA | 1821] | 1889] | GUCUAAACUA |
| | | | | | | TCAATATGTT | | | UCAAUAUGUU |
| | | | | | | GCATACAGTC | | | GCAUACAGUC |
| | | | | | | AGATAGCTAC | | | AGAUAGCUAC |
| | | | | | | TGCTAGGCAA | | | UGCUAGGCAA |
| | | | | | | TCCTTCCCTC | | | UCCUUCCCUC |
| | | | | | | GATAAATGTC | | | GAUAAAUGUC |
| | | | | | | TTGGCATCGT | | | UUGGCAUCGU |
| | | | | | | TTGCTTTG | | | UUGCUUUG |
| | | | | | | [SEQ ID | | | [SEQ ID |
| | | | | | | NO: | | | NO: |
| | | | | | | 1787] | | | 1984] |
| miR- | 1162 | −2.2647 | −0.33024 | AACTGTACCA | CAGACTTTGT | GGCTACAGAG | AACUGUACCA | CAGACUUUGU | GGCUACAGAG |
| 122 | | | | CAACAAAGTC | TATGGTACAT | TTTCCTTAGC | CAACAAAGUC | UAUGGUACAU | UUUCCUUAGC |
| | | | | TG | GT | AGAGCTGAAC | UG | GU | AGAGCUGAAC |
| | | | | [SEQ ID | [SEQ ID | TGTACCACAA | [SEQ ID | [SEQ ID | UGUACCACAA |
| | | | | NO: | NO: | CAAAGTCTGT | NO: | NO: | CAAAGUCUGU |
| | | | | 652] | 1701] | GTCTAAACTA | 1180] | 1896] | GUCUAAACUA |
| | | | | | | TCAGACTTTG | | | UCAGACUUUG |
| | | | | | | TTATGGTACA | | | UUAUGGUACA |
| | | | | | | TGTTAGCTAC | | | UGUUAGCUAC |
| | | | | | | TGCTAGGCAA | | | UGCUAGGCAA |
| | | | | | | TCCTTCCCTC | | | UCCUUCCCUC |
| | | | | | | GATAAATGTC | | | GAUAAAUGUC |
| | | | | | | TTGGCATCGT | | | UUGGCAUCGU |
| | | | | | | TTGCTTTG | | | UUGCUUUG |
| | | | | | | [SEQ ID | | | [SEQ ID |
| | | | | | | NO: | | | NO: |
| | | | | | | 1794] | | | 1991] |
| miR- | 3301 | −2.1436 | 0.009402 | TGAACAAGGG | TCCCAAATCA | GGCTACAGAG | UGAACAAGGG | UCCCAAAUCA | GGCUACAGAG |
| 122 | | | | GCTGATTTGG | GACCCTTGTG | TTTCCTTAGC | GCUGAUUUGG | GACCCUUGUG | UUUCCUUAGC |
| | | | | GA | AA | AGAGCTGTGA | GA | AA | AGAGCUGUGA |
| | | | | [SEQ ID | [SEQ ID | ACAAGGGGCT | [SEQ ID | [SEQ ID | ACAAGGGGCU |
| | | | | NO: | NO: | GATTTGGGAT | NO: | NO: | GAUUUGGGAU |
| | | | | 687] | 1702] | GTCTAAACTA | 1215] | 1897] | GUCUAAACUA |
| | | | | | | TTCCCAAATC | | | UUCCCAAAUC |
| | | | | | | AGACCCTTGT | | | AGACCCUUGU |
| | | | | | | GAATAGCTAC | | | GAAUAGCUAC |
| | | | | | | TGCTAGGCAA | | | UGCUAGGCAA |
| | | | | | | TCCTTCCCTC | | | UCCUUCCCUC |
| | | | | | | GATAAATGTC | | | GAUAAAUGUC |
| | | | | | | TTGGCATCGT | | | UUGGCAUCGU |
| | | | | | | TTGCTTTG | | | UUGCUUUG |
| | | | | | | [SEQ ID | | | [SEQ ID |
| | | | | | | NO: | | | NO: |
| | | | | | | 2026] | | | 2027] |
| miR- | 3330 | −2.06194 | 0.224544 | TATGCTGAGA | CCACATTATC | GGCTACAGAG | UAUGCUGAGA | CCACAUUAUC | GGCUACAGAG |
| 122 | | | | CTGATAATGT | AATCTCAGCC | TTTCCTTAGC | CUGAUAAUGU | AAUCUCAGCC | UUUCCUUAGC |
| | | | | GG | GA | AGAGCTGTAT | GG | GA | AGAGCUGUAU |
| | | | | [SEQ ID | [SEQ ID | GCTGAGACTG | [SEQ ID | [SEQ ID | GCUGAGACUG |
| | | | | NO: | NO: | ATAATGTGGT | NO: | NO: | AUAAUGUGGU |
| | | | | 1614] | 2028] | GTCTAAACTA | 1811] | 2030] | GUCUAAACUA |
| | | | | | | TCCACATTAT | | | UCCACAUUAU |
| | | | | | | CAATCTCAGC | | | CAAUCUCAGC |
| | | | | | | CGATAGCTAC | | | CGAUAGCUAC |
| | | | | | | TGCTAGGCAA | | | UGCUAGGCAA |
| | | | | | | TCCTTCCCTC | | | UCCUUCCCUC |
| | | | | | | GATAAATGTC | | | GAUAAAUGUC |
| | | | | | | TTGGCATCGT | | | UUGGCAUCGU |
| | | | | | | TTGCTTTG | | | UUGCUUUG |
| | | | | | | [SEQ ID | | | [SEQ ID |
| | | | | | | NO: | | | NO: |
| | | | | | | 2029] | | | 2031] |
| miR- | 3272 | −2.04615 | −0.54779 | AGGACTGTAG | GCAATATGTT | GGCTACAGAG | AGGACUGUAG | GCAAUAUGUU | GGCUACAGAG |
| 122 | | | | GCAACATATT | GACTACAGTA | TTTCCTTAGC | GCAACAUAUU | GACUACAGUA | UUUCCUUAGC |
| | | | | GC | AT | AGAGCTGAGG | GC | AU | AGAGCUGAGG |
| | | | | [SEQ ID | [SEQ ID | ACTGTAGGCA | [SEQ ID | [SEQ ID | ACUGUAGGCA |
| | | | | NO: | NO: | ACATATTGCT | NO: | NO: | ACAUAUUGCU |
| | | | | 1618] | 2032] | GTCTAAACTA | 1814] | 2034] | GUCUAAACUA |
| | | | | | | TGCAATATGT | | | UGCAAUAUGU |
| | | | | | | TGACTACAGT | | | UGACUACAGU |
| | | | | | | AATTAGCTAC | | | AAUUAGCUAC |
| | | | | | | TGCTAGGCAA | | | UGCUAGGCAA |
| | | | | | | TCCTTCCCTC | | | UCCUUCCCUC |
| | | | | | | GATAAATGTC | | | GAUAAAUGUC |
| | | | | | | TTGGCATCGT | | | UUGGCAUCGU |
| | | | | | | TTGCTTTG | | | UUGCUUUG |
| | | | | | | [SEQ ID | | | [SEQ ID |
| | | | | | | NO: | | | NO: |
| | | | | | | 2033] | | | 2261 |
| miR- | 3133 | −2.03169 | 0.05703 | TATGTCTTGG | CAGTGAATCA | GGCTACAGAG | UAUGUCUUGG | CAGUGAAUCA | GGCUACAGAG |
| 122 | | | | CTTGATTCAC | AACCAAGACC | TTTCCTTAGC | CUUGAUUCAC | AACCAAGACC | UUUCCUUAGC |
| | | | | TG | GA | AGAGCTGTAT | UG | GA | AGAGCUGUAU |
| | | | | [SEQ ID | [SEQ ID | GTCTTGGCTT | [SEQ ID | [SEQ ID | GUCUUGGCUU |
| | | | | NO: | NO: | GATTCACTGT | NO: | NO: | GAUUCACUGU |
| | | | | 1624] | 1666] | GTCTAAACTA | 1819] | 1861] | GUCUAAACUA |
| | | | | | | TCAGTGAATC | | | UCAGUGAAUC |
| | | | | | | AAACCAAGAC | | | AAACCAAGAC |
| | | | | | | CGATAGCTAC | | | CGAUAGCUAC |
| | | | | | | TGCTAGGCAA | | | UGCUAGGCAA |
| | | | | | | TCCTTCCCTC | | | UCCUUCCCUC |
| | | | | | | GATAAATGTC | | | GAUAAAUGUC |
| | | | | | | TTGGCATCGT | | | UUGGCAUCGU |
| | | | | | | TTGCTTTG | | | UUGCUUUG |
| | | | | | | [SEQ ID | | | [SEQ ID |
| | | | | | | NO: | | | NO: |
| | | | | | | 2262 | | | 2263 |
| miR- | 1755 | −2.60043 | −0.03541 | TCGGGTTGAA | CACACTTCAG | GGCTACAGAG | UCGGGUUGAA | CACACUUCAG | GGCUACAGAG |
| 122_M | | | | ATCTGAAGTG | ACTTCAACCA | TTTGCTTAGC | AUCUGAAGUG | ACUUCAACCA | UUUGCUUAGC |
| | | | | TG | TA | AGAGCTGTCG | UG | UA | AGAGCUGUCG |
| | | | | [SEQ ID | [SEQ ID | GGTTGAAATC | [SEQ ID | [SEQ ID | GGUUGAAAUC |
| | | | | NO: | NO: | TGAAGTGTGT | NO: | NO: | UGAAGUGUGU |
| | | | | 657] | 1646] | GTCTAAACTA | 1185] | 1841] | GUCUAAACUA |
| | | | | | | TCACACTTCA | | | UCACACUUCA |
| | | | | | | GACTTCAACC | | | GACUUCAACC |
| | | | | | | ATATAGCTAC | | | AUAUAGCUAC |
| | | | | | | TGCTAGGCCA | | | UGCUAGGCCA |
| | | | | | | TCCTTCCCTC | | | UCCUUCCCUC |
| | | | | | | GATAAATGTC | | | GAUAAAUGUC |
| | | | | | | TTGGCATCGT | | | UUGGCAUCGU |
| | | | | | | TTGCTTTG | | | UUGCUUUG |
| | | | | | | [SEQ ID | | | [SEQ ID |
| | | | | | | NO: | | | NO: |
| | | | | | | 1725] | | | 1922] |
| miR- | 3133 | −2.43848 | 0.197681 | TATGTCTTGG | CAGTGAATCA | GGCTACAGAG | UAUGUCUUGG | CAGUGAAUCA | GGCUACAGAG |
| 122_M | | | | CTTGATTCAC | AACCAAGACC | TTTGCTTAGC | CUUGAUUCAC | AACCAAGACC | UUUGCUUAGC |
| | | | | TG | GA | AGAGCTGTAT | UG | GA | AGAGCUGUAU |
| | | | | [SEQ ID | [SEQ ID | GTCTTGGCTT | [SEQ ID | [SEQ ID | GUCUUGGCUU |
| | | | | NO: | NO: | GATTCACTGT | NO: | NO: | GAUUCACUGU |
| | | | | 1624] | 1666] | GTCTAAACTA | 1819] | 1861] | GUCUAAACUA |
| | | | | | | TCAGTGAATC | | | UCAGUGAAUC |
| | | | | | | AAACCAAGAC | | | AAACCAAGAC |
| | | | | | | CGATAGCTAC | | | CGAUAGCUAC |
| | | | | | | TGCTAGGCCA | | | UGCUAGGCCA |
| | | | | | | TCCTTCCCTC | | | UCCUUCCCUC |
| | | | | | | GATAAATGTC | | | GAUAAAUGUC |
| | | | | | | TTGGCATCGT | | | UUGGCAUCGU |
| | | | | | | TTGCTTTG | | | UUGCUUUG |
| | | | | | | [SEQ ID | | | [SEQ ID |
| | | | | | | NO: | | | NO: |
| | | | | | | 1752] | | | 1949] |
| miR- | 3302 | −2.40308 | −0.14782 | TTGAACAAGG | CCCAAATCAG | GGCTACAGAG | UUGAACAAGG | CCCAAAUCAG | GGCUACAGAG |
| 122_M | | | | GGCTGATTTG | CACCTTGTTA | TTTGCTTAGC | GGCUGAUUUG | CACCUUGUUA | UUUGCUUAGC |
| | | | | GG | CA | AGAGCTGTTG | GG | CA | AGAGCUGUUG |
| | | | | [SEQ ID | [SEQ ID | AACAAGGGGC | [SEQ ID | [SEQ ID | AACAAGGGGC |
| | | | | NO: | NO: | TGATTTGG | NO: | NO: | UGAUUUGGG |
| | | | | 688] | 1675] | GTGTCTAAAC | 1216] | 1870] | UGUCUAAACU |
| | | | | | | TATCCCAAAT | | | AUCCCAAAUC |
| | | | | | | CAGCACCTTG | | | AGCACCUUGU |
| | | | | | | TTACATAGCT | | | UACAUAGCUA |
| | | | | | | ACTGCTAGGC | | | CUGCUAGGCC |
| | | | | | | CATCCTTCCC | | | AUCCUUCCCU |
| | | | | | | TCGATAAATG | | | CGAUAAAUGU |
| | | | | | | TCTTGGCATC | | | CUUGGCAUCG |
| | | | | | | GTTTGCTT | | | UUUGCUUUG |
| | | | | | | TG | | | [SEQ ID |
| | | | | | | [SEQ ID | | | NO: |
| | | | | | | NO: | | | 1962] |
| | | | | | | 1765] | | | |
| miR- | 3301 | −2.26423 | 0.053866 | TGAACAAGGG | TCCCAAATCA | GGCTACAGAG | UGAACAAGGG | UCCCAAAUCA | GGCUACAGAG |
| 122_M | | | | GCTGATTTGG | GACCCTTGTG | TTTGCTTAGC | GCUGAUUUGG | GACCCUUGUG | UUUGCUUAGC |
| | | | | GA | AA | AGAGCTGTGA | GA | AA | AGAGCUGUGA |
| | | | | [SEQ ID | [SEQ ID | ACAAGGGGCT | [SEQ ID | [SEQ ID | ACAAGGGGCU |
| | | | | NO: | NO: | GATTTGGGAT | NO: | NO: | GAUUUGGGAU |
| | | | | 687] | 1702] | GTCTAAACTA | 1215] | 1897] | GUCUAAACUA |
| | | | | | | TTCCCAAATC | | | UUCCCAAAUC |
| | | | | | | AGACCCTTGT | | | AGACCCUUGU |
| | | | | | | GAATAGCTAC | | | GAAUAGCUAC |
| | | | | | | TGCTAGGCCA | | | UGCUAGGCCA |
| | | | | | | TCCTTCCCTC | | | UCCUUCCCUC |
| | | | | | | GATAAATGTC | | | GAUAAAUGUC |
| | | | | | | TTGGCATCGT | | | UUGGCAUCGU |
| | | | | | | TTGCTTTG | | | UUGCUUUG |
| | | | | | | [SEQ ID | | | [SEQ ID |
| | | | | | | NO: | | | NO: |
| | | | | | | 1795] | | | 1992] |
| miR- | 3132 | −2.24518 | 0.049515 | ATGTCTTGGC | CCAGTGAATC | GGCTACAGAG | AUGUCUUGGC | CCAGUGAAUC | GGCUACAGAG |
| 122M | | | | TTGATTCACT | ACGCCAAGAA | TTTGCTTAGC | UUGAUUCACU | ACGCCAAGAA | UUUGCUUAGC |
| | | | | GG | CT | AGAGCTGATG | GG | CU | AGAGCUGAUG |
| | | | | [SEQ ID | [SEQ ID | TCTTGGCTTG | [SEQ ID | [SEQ ID | UCUUGGCUUG |
| | | | | NO: | NO: | ATTCACTGGT | NO: | NO: | AUUCACUGGU |
| | | | | 1631] | 1707] | GTCTAAACTA | 1826] | 1904] | GUCUAAACUA |
| | | | | | | TCCAGTGAAT | | | UCCAGUGAAU |
| | | | | | | CACGCCAAGA | | | CACGCCAAGA |
| | | | | | | ACTTAGCTAC | | | ACUUAGCUAC |
| | | | | | | TGCTAGGCCA | | | UGCUAGGCCA |
| | | | | | | TCCTTCCCTC | | | UCCUUCCCUC |
| | | | | | | GATAAATGTC | | | GAUAAAUGUC |
| | | | | | | TTGGCATCGT | | | UUGGCAUCGU |
| | | | | | | TTGCTTTG | | | UUGCUUUG |
| | | | | | | [SEQ ID | | | [SEQ ID |
| | | | | | | NO: | | | NO: |
| | | | | | | 1804] | | | 2001] |
| miR- | 3341 | −2.10774 | 0.117999 | ATAGACATGA | GTCTCAGCAT | GGCTACAGAG | AUAGACAUGA | GUCUCAGCAU | GGCUACAGAG |
| 122_M | | | | GGATGCTGAG | CATCATGTCG | TTTGCTTAGC | GGAUGCUGAG | CAUCAUGUCG | UUUGCUUAGC |
| | | | | AC | CT | AGAGCTGATA | AC | CU | AGAGCUGAUA |
| | | | | [SEQ ID | [SEQ ID | GACATGAGGA | [SEQ ID | [SEQ ID | GACAUGAGGA |
| | | | | NO: | NO: | TGCTGAGACT | NO: | NO: | UGCUGAGACU |
| | | | | 1617] | 1657] | GTCTAAACTA | 1813] | 1852] | GUCUAAACUA |
| | | | | | | TGTCTCAGCA | | | UGUCUCAGCA |
| | | | | | | TCAT | | | UCAU |
| | | | | | | CATGTCGCTT | | | CAUGUCGCUU |
| | | | | | | AGCTACTGCT | | | AGCUACUGCU |
| | | | | | | AGGCCATCCT | | | AGGCCAUCCU |
| | | | | | | TCCCTCGATA | | | UCCCUCGAUA |
| | | | | | | AATGTCTTGG | | | AAUGUCUUGG |
| | | | | | | CATCGTTTGC | | | CAUCGUUUGC |
| | | | | | | TTTG | | | UUUG |
| | | | | | | [SEQ ID | | | [SEQ ID |
| | | | | | | NO: | | | NO: |
| | | | | | | 2264] | | | 2265] |
| miR- | 3332 | −2.06545 | −0.17324 | AGGATGCTGA | ACATTATCAG | GGCTACAGAG | AGGAUGCUGA | ACAUUAUCAG | GGCUACAGAG |
| 122M | | | | GACTGATAAT | TATCAGCATA | TTTGCTTAGC | GACUGAUAAU | UAUCAGCAUA | UUUGCUUAGC |
| | | | | GT | AT | AGAGCTGAGG | GU | AU | AGAGCUGAGG |
| | | | | [SEQ ID | [SEQ ID | ATGCTGAGAC | [SEQ ID | [SEQ ID | AUGCUGAGAC |
| | | | | NO: | NO: | TGATAATGTT | NO: | NO: | UGAUAAUGUU |
| | | | | 1623] | 1655] | GTCTAAACTA | 1818] | 1850] | GUCUAAACUA |
| | | | | | | TACATTATCA | | | UACAUUAUCA |
| | | | | | | GTATCAGCAT | | | GUAUCAGCAU |
| | | | | | | AATTAGCTAC | | | AAUUAGCUAC |
| | | | | | | TGCTAGGCCA | | | UGCUAGGCCA |
| | | | | | | TCCTTCCCTC | | | UCCUUCCCUC |
| | | | | | | GATAAATGTC | | | GAUAAAUGUC |
| | | | | | | TTGGCATCGT | | | UUGGCAUCGU |
| | | | | | | TTGCTTTG | | | UUGCUUUG |
| | | | | | | [SEQ ID | | | [SEQ ID |
| | | | | | | NO: | | | NO: |
| | | | | | | 2266] | | | 2267] |
| miR- | 3272 | −2.0434 | −0.02715 | AGGACTGTAG | GCAATATGTT | GGCTACAGAG | AGGACUGUAG | GCAAUAUGUU | GGCUACAGAG |
| 122M | | | | GCAACATATT | GACTACAGTA | TTTGCTTAGC | GCAACAUAUU | GACUACAGUA | UUUGCUUAGC |
| | | | | GC | AT | AGAGCTGAGG | GC | AU | AGAGCUGAGG |
| | | | | [SEQ ID | [SEQ ID | ACTGTAGGCA | [SEQ ID | [SEQ ID | ACUGUAGGCA |
| | | | | NO: | NO: | ACATATTGCT | NO: | NO: | ACAUAUUGCU |
| | | | | 1618] | 2032] | GTCTAAACTA | 1814] | 2034] | GUCUAAACUA |
| | | | | | | TGCAATATGT | | | UGCAAUAUGU |
| | | | | | | TGACTACAGT | | | UGACUACAGU |
| | | | | | | AATTAGCTAC | | | AAUUAGCUAC |
| | | | | | | TGCTAGGCCA | | | UGCUAGGCCA |
| | | | | | | TCCTTCCCTC | | | UCCUUCCCUC |
| | | | | | | GATAAATGTC | | | GAUAAAUGUC |
| | | | | | | TTGGCATCGT | | | UUGGCAUCGU |
| | | | | | | TTGCTTTG | | | UUGCUUUG |
| | | | | | | [SEQ ID | | | [SEQ ID |
| | | | | | | NO: | | | NO: |
| | | | | | | 2268] | | | 2035] |
| miR- | 3338 | −1.99732 | −0.07815 | TACATGAGGA | TCAGTCTCAG | GGCTACAGAG | UACAUGAGGA | UCAGUCUCAG | GGCUACAGAG |
| 122_M | | | | TGCTGAGACT | CCTCCTCATT | TTTGCTTAGC | UGCUGAGACU | CCUCCUCAUU | UUUGCUUAGC |
| | | | | GA | GA | AGAGCTGTAC | GA | GA | AGAGCUGUAC |
| | | | | [SEQ ID | [SEQ ID | ATGAGGATGC | [SEQ ID | [SEQ ID | AUGAGGAUGC |
| | | | | NO: | NO: | TGAGACTGAT | NO: | NO: | UGAGACUGAU |
| | | | | 1620] | 2036] | GTCTAAACTA | 314] | 2038] | GUCUAAACUA |
| | | | | | | TTCAGTCTCA | (Same | | UUCAGUCUCA |
| | | | | | | GCCTCCTCAT | guide | | GCCUCCUCAU |
| | | | | | | TGATAGC | as | | UGAUAGC |
| | | | | | | TACTGCTAGG | XD- | | UACUGCUAGG |
| | | | | | | CCATCCTTCC | 14893) | | CCAUCCUUCC |
| | | | | | | CTCGATAAAT | | | CUCGAUAAAU |
| | | | | | | GTCTTGGCAT | | | GUCUUGGCAU |
| | | | | | | CGTTTGCTTT | | | CGUUUGCUUU |
| | | | | | | G | | | G |
| | | | | | | [SEQ ID | | | [SEQ ID |
| | | | | | | NO: | | | NO: |
| | | | | | | 2037] | | | 2039] |
| miR- | 1162 | −1.99443 | −0.12785 | AACTGTACCA | CAGACTTTGT | GGCTACAGAG | AACUGUACCA | CAGACUUUGU | GGCUACAGAG |
| 122M | | | | CAACAAAGTC | TATGGTACAT | TTTGCTTAGC | CAACAAAGUC | UAUGGUACAU | UUUGCUUAGC |
| | | | | TG | GT | AGAGCTGAAC | UG | GU | AGAGCUGAAC |
| | | | | [SEQ ID | [SEQ ID | TGTACCACAA | [SEQ ID | [SEQ ID | UGUACCACAA |
| | | | | NO: | NO: | CAAAGTCTGT | NO: | NO: | CAAAGUCUGU |
| | | | | 652] | 1701] | GTCTAAACTA | 1180] | 1896] | GUCUAAACUA |
| | | | | | | TCAGACTTTG | | | UCAGACUUUG |
| | | | | | | TTATGGTACA | | | UUAUGGUACA |
| | | | | | | TGTTAGCTAC | | | UGUUAGCUAC |
| | | | | | | TGCTAGGCCA | | | UGCUAGGCCA |
| | | | | | | TCCTTCCCTC | | | UCCUUCCCUC |
| | | | | | | GATAAATGTC | | | GAUAAAUGUC |
| | | | | | | TTGGCATCGT | | | UUGGCAUCGU |
| | | | | | | TTGCTTTG | | | UUGCUUUG |
| | | | | | | [SEQ ID | | | [SEQ ID |
| | | | | | | NO: | | | NO: |
| | | | | | | 2040] | | | 2041] |
| miR- | 2928 | −1.82454 | 0.049772 | TCTGAGAGAA | ACGCCACGTT | TTCCTTCCTC | UCUGAGAGAA | ACGCCACGUU | UUCCUUCCUC |
| 124 | | | | GGAACGTGGG | CATTCGATCA | AGGAGAAAGG | GGAACGUGGG | CAUUCGAUCA | AGGAGAAAGG |
| | | | | TT | GA | CCTCTCTCAC | UU | GA | CCUCUCUCAC |
| | | | | [SEQ ID | [SEQ ID | GCCACGTTCA | [SEQ ID | [SEQ ID | GCCACGUUCA |
| | | | | NO: | NO: | TTCGATCAGA | NO: | NO: | UUCGAUCAGA |
| | | | | 668] | 2042] | ATTTAAATGT | 1196] | 2044] | AUUUAAAUGU |
| | | | | | | CCATACAATT | | | CCAUACAAUU |
| | | | | | | CTGAGAGAAG | | | CUGAGAGAAG |
| | | | | | | GAACGTGGGT | | | GAACGUGGGU |
| | | | | | | TGAATGGGGC | | | UGAAUGGGGC |
| | | | | | | TGGCTGAGCA | | | UGGCUGAGCA |
| | | | | | | CCGTGGGTCG | | | CCGUGGGUCG |
| | | | | | | GCGAGGGCCC | | | GCGAGGGCCC |
| | | | | | | GCCAAGGA | | | GCCAAGGA |
| | | | | | | [SEQ ID | | | [SEQ ID |
| | | | | | | NO: | | | NO: |
| | | | | | | 2043] | | | 2045] |
| miR- | 3043 | −1.7881 | 0.031105 | TTTGGTGCAA | AAGCTGTTTG | TTCCTTCCTC | UUUGGUGCAA | AAGCUGUUUG | UUCCUUCCUC |
| 124 | | | | AACAAACAGG | TGTTGACCCA | AGGAGAAAGG | AACAAACAGG | UGUUGACCCA | AGGAGAAAGG |
| | | | | CT | AA | CCTCTCTCAA | CU | AA | CCUCUCUCAA |
| | | | | [SEQ ID | [SEQ ID | GCTGTTTGTG | [SEQ ID | [SEQ ID | GCUGUUUGUG |
| | | | | NO: | NO: | TTGACCCAAA | NO: | NO: | UUGACCCAAA |
| | | | | 1615] | 2046] | ATTTAAATGT | 1812] | 2048] | AUUUAAAUGU |
| | | | | | | CCATACAATT | | | CCAUACAAUU |
| | | | | | | TTGGTGCAAA | | | UUGGUGCAAA |
| | | | | | | ACAAACAGGC | | | ACAAACAGGC |
| | | | | | | TGAATGGGGC | | | UGAAUGGGGC |
| | | | | | | TGGCTGAGCA | | | UGGCUGAGCA |
| | | | | | | CCGTGGGTCG | | | CCGUGGGUCG |
| | | | | | | GCGAGGGCCC | | | GCGAGGGCCC |
| | | | | | | GCCAAGGA | | | GCCAAGGA |
| | | | | | | [SEQ ID | | | [SEQ ID |
| | | | | | | NO: | | | NO: |
| | | | | | | 2047] | | | 2049] |
| miR- | 3256 | −1.65786 | 0.035613 | TATTGCGTGG | CCGCAGCTTA | TTCCTTCCTC | UAUUGCGUGG | CCGCAGCUUA | UUCCUUCCUC |
| 124 | | | | AGTAAGCTGG | CGCCAAACAA | AGGAGAAAGG | AGUAAGCUGG | CGCCAAACAA | AGGAGAAAGG |
| | | | | TG | TA | CCTCTCTCCC | UG | UA | CCUCUCUCCC |
| | | | | [SEQ ID | [SEQ ID | GCAGCTTACG | [SEQ ID | [SEQ ID | GCAGCUUACG |
| | | | | NO: | NO: | CCAAACAATA | NO: | NO: | CCAAACAAUA |
| | | | | 618] | 2050] | ATTTAAATGT | 308] | 2052] | AUUUAAAUGU |
| | | | | | | CCATACAATT | (Same | | CCAUACAAUU |
| | | | | | | ATTGCGTGGA | as | | AUUGCGUGGA |
| | | | | | | GTAAGCTGGT | XD- | | GUAAGCUGGU |
| | | | | | | GGAATGGGGC | 14890) | | GGAAUGGGGC |
| | | | | | | TGGCTGAGCA | | | UGGCUGAGCA |
| | | | | | | CCGTGGGTCG | | | CCGUGGGUCG |
| | | | | | | GCGAGGGCCC | | | GCGAGGGCCC |
| | | | | | | GCCAAGGA | | | GCCAAGGA |
| | | | | | | [SEQ ID | | | [SEQ ID |
| | | | | | | NO: | | | NO: |
| | | | | | | 2051] | | | 2053] |
| miR- | 2353 | −1.64493 | 0.114403 | TTCGATGCAG | CAGCTGCTAG | TTCCTTCCTC | UUCGAUGCAG | CAGCUGCUAG | UUCCUUCCUC |
| 124 | | | | GACTAGCAGG | TACTGACTCG | AGGAGAAAGG | GACUAGCAGG | UACUGACUCG | AGGAGAAAGG |
| | | | | CG | AA | CCTCTCTCCA | CG | AA | CCUCUCUCCA |
| | | | | [SEQ ID | [SEQ ID | GCTGCTAGTA | [SEQ ID | [SEQ ID | GCUGCUAGUA |
| | | | | NO: | NO: | CTGACTCGAA | NO: | NO: | CUGACUCGAA |
| | | | | 612] | 2054] | ATTTAAATGT | 166] | 2056] | AUUUAAAUGU |
| | | | | | | CCATACAATT | (Same | | CCAUACAAUU |
| | | | | | | TCGATGCAGG | as | | UCGAUGCAGG |
| | | | | | | ACTAGCAGGC | XD- | | ACUAGCAGGC |
| | | | | | | GGAATGGGGC | 14819) | | GGAAUGGGGC |
| | | | | | | TGGCTGAGCA | | | UGGCUGAGCA |
| | | | | | | CCGTGGGTCG | | | CCGUGGGUCG |
| | | | | | | GCGAGGGCCC | | | GCGAGGGCCC |
| | | | | | | GCCAAGGA | | | GCCAAGGA |
| | | | | | | [SEQ ID | | | [SEQ ID |
| | | | | | | NO: | | | NO: |
| | | | | | | 2055] | | | 2057] |
| miR- | 1231 | −1.5099 | −0.27848 | TTCACTTTAG | CCCCTATCAG | TTCCTTCCTC | UUCACUUUAG | CCCCUAUCAG | UUCCUUCCUC |
| 124 | | | | CACTGATAGC | TTCTACCGTG | AGGAGAAAGG | CACUGAUAGC | UUCUACCGUG | AGGAGAAAGG |
| | | | | AG | AA | CCTCTCTCCC | AG | AA | CCUCUCUCCC |
| | | | | [SEQ ID | [SEQ ID | CCTATCAGTT | [SEQ ID | [SEQ ID | CCUAUCAGUU |
| | | | | NO: | NO: | CTACCGTGAA | NO: | NO: | CUACCGUGAA |
| | | | | 1627] | 2058] | ATTTAAATGT | 1825] | 2060] | AUUUAAAUGU |
| | | | | | | CCATACAATT | | | CCAUACAAUU |
| | | | | | | TCACTTTAGC | | | UCACUUUAGC |
| | | | | | | ACTGATAGCA | | | ACUGAUAGCA |
| | | | | | | GGAATGGGGC | | | GGAAUGGGGC |
| | | | | | | TGGCTGAGCA | | | UGGCUGAGCA |
| | | | | | | CCG | | | CCGUG |
| | | | | | | TGGGTCGGCG | | | GGUCGGCGAG |
| | | | | | | AGGGCCCGCC | | | GGCCCGCCAA |
| | | | | | | AAGGA | | | GGA |
| | | | | | | [SEQ ID | | | [SEQ ID |
| | | | | | | NO: | | | NO: |
| | | | | | | 2059] | | | 2061] |
| miR- | 3546 | −1.46171 | −0.28183 | TCGCATACTG | TAGCTTGCTC | TTCCTTCCTC | UCGCAUACUG | UAGCUUGCUC | UUCCUUCCUC |
| 124 | | | | CTGAGCAAGG | ATCAGGCTGC | AGGAGAAAGG | CUGAGCAAGG | AUCAGGCUGC | AGGAGAAAGG |
| | | | | GA | GA | CCTCTCTCTA | GA | GA | CCUCUCUCUA |
| | | | | [SEQ ID | [SEQ ID | GCTTGCTCAT | [SEQ ID | [SEQ ID | GCUUGCUCAU |
| | | | | NO: | NO: | CAGGCTGCGA | NO: | NO: | CAGGCUGCGA |
| | | | | 2062] | 2063] | ATTTAAATGT | 2065] | 2066] | AUUUAAAUGU |
| | | | | | | CCATACAATT | | | CCAUACAAUU |
| | | | | | | CGCATACTGC | | | CGCAUACUGC |
| | | | | | | TGAGCAAGGG | | | UGAGCAAGGG |
| | | | | | | AGAATGGGGC | | | AGAAUGGGGC |
| | | | | | | TGGCTGAGCA | | | UGGCUGAGCA |
| | | | | | | CCGTGGGTCG | | | CCGUGGGUCG |
| | | | | | | GCGAGGGCCC | | | GCGAGGGCCC |
| | | | | | | GCCAAGGA | | | GCCAAGGA |
| | | | | | | [SEQ ID | | | [SEQ ID |
| | | | | | | NO: | | | NO: |
| | | | | | | 2064] | | | 2067] |
| miR- | 893 | −1.42389 | −0.31114 | TTTGTTACTG | CCCAGGTCGA | TTCCTTCCTC | UUUGUUACUG | CCCAGGUCGA | UUCCUUCCUC |
| 124 | | | | TTTCGACCTC | ACCAGGCACA | AGGAGAAAGG | UUUCGACCUC | ACCAGGCACA | AGGAGAAAGG |
| | | | | TG | AA | CCTCTCTCCC | UG | AA | CCUCUCUCCC |
| | | | | [SEQ ID | [SEQ ID | CAGGTCGAAC | [SEQ ID | [SEQ ID | CAGGUCGAAC |
| | | | | NO: | NO: | CAGGCACAAA | NO: | NO: | CAGGCACAAA |
| | | | | 2012] | 2068] | ATTTAAATGT | 2015] | 2070] | AUUUAAAUGU |
| | | | | | | CCATACAATT | | | CCAUACAAUU |
| | | | | | | TTGTTACTGT | | | UUGUUACUGU |
| | | | | | | TTCGACCTCT | | | UUCGACCUCU |
| | | | | | | GGAATGGGGC | | | GGAAUGGGGC |
| | | | | | | TGGCTGAGCA | | | UGGCUGAGCA |
| | | | | | | CCGTGGGTCG | | | CCGUGGGUCG |
| | | | | | | GCGAGGGCCC | | | GCGAGGGCCC |
| | | | | | | GCCAAGGA | | | GCCAAGGA |
| | | | | | | [SEQ ID | | | [SEQ ID |
| | | | | | | NO: | | | NO: |
| | | | | | | 2069] | | | 2071] |
| miR- | 2602 | −1.34373 | 0.049733 | TTTAGTAGTT | ACACTATGGA | TTCCTTCCTC | UUUAGUAGUU | ACACUAUGGA | UUCCUUCCUC |
| 124 | | | | GATCCATAGA | TAAACGCCTA | AGGAGAAAGG | GAUCCAUAGA | UAAACGCCUA | AGGAGAAAGG |
| | | | | TT | AA | CCTCTCTCAC | UU | AA | CCUCUCUCAC |
| | | | | [SEQ ID | [SEQ ID | ACTATGGATA | [SEQ ID | [SEQ ID | ACUAUGGAUA |
| | | | | NO: | NO: | AACGCCTAAA | NO: | NO: | AACGCCUAAA |
| | | | | 1616] | 2072] | ATTTAAATGT | 202] | 2074] | AUUUAAAUGU |
| | | | | | | CCATACAATT | (Same | | CCAUACAAUU |
| | | | | | | TTAGTAGTTG | guide | | UUAGUAGUUG |
| | | | | | | ATCCATAGAT | as | | AUCCAUAGAU |
| | | | | | | TGAATGGGGC | XD- | | UGAAUGGGGC |
| | | | | | | TGGCTGAGCA | 14837) | | UGGCUGAGCA |
| | | | | | | CCGTGGGTCG | | | CCG |
| | | | | | | GCG | | | UGGGU |
| | | | | | | AGGGCCCGCC | | | CGGCG |
| | | | | | | AAGGA | | | AGGGCCCGCC |
| | | | | | | [SEQ ID | | | AAGGA |
| | | | | | | NO: | | | [SEQ ID |
| | | | | | | 2073] | | | NO: |
| | | | | | | | | | 2075] |
| miR- | 2944 | −1.33679 | 0.075742 | TTAGTAGAAG | CCGAGCCAAA | TTCCTTCCTC | UUAGUAGAAG | CCGAGCCAAA | UUCCUUCCUC |
| 124 | | | | GCTTTGGCTG | GACTTACACT | AGGAGAAAGG | GCUUUGGCUG | GACUUACACU | AGGAGAAAGG |
| | | | | AG | AA | CCTCTCTCCC | AG | AA | CCUCUCUCCC |
| | | | | [SEQ ID | [SEQ ID | GAGCCAAAGA | [SEQ ID | [SEQ ID | GAGCCAAAGA |
| | | | | NO: | NO: | CTTACACTAA | NO: | NO: | CUUACACUAA |
| | | | | 684] | 2076] | ATTTAAATGT | 246] | 2078] | AUUUAAAUGU |
| | | | | | | CCATACAATT | (Same | | CCAUACAAUU |
| | | | | | | TAGTAGAAGG | guide | | UAGUAGAAGG |
| | | | | | | CTTTGGCTGA | as | | CUUUGGCUGA |
| | | | | | | GGAATGGGGC | XD- | | GGAAUGGGGC |
| | | | | | | TGGCTGAGCA | 14859) | | UGGCUGAGCA |
| | | | | | | CCGTGGGTCG | | | CCGUGGGUCG |
| | | | | | | GCGAGGGCCC | | | GCGAGGGCCC |
| | | | | | | GCCAAGGA | | | GCCAAGGA |
| | | | | | | [SEQ ID | | | [SEQ ID |
| | | | | | | NO: | | | NO: |
| | | | | | | 2077] | | | 2079] |
| miR- | 3270 | −1.2645 | 0.050943 | TACTGTAGGC | AACCAATATG | TTCCTTCCTC | UACUGUAGGC | AACCAAUAUG | UUCCUUCCUC |
| 124 | | | | AACATATTGC | TGGCCGCCAG | AGGAGAAAGG | AACAUAUUGC | UGGCCGCCAG | AGGAGAAAGG |
| | | | | GT | TA | CCTCTCTCAA | GU | UA | CCUCUCUCAA |
| | | | | [SEQ ID | [SEQ ID | CCAATATGTG | [SEQ ID | [SEQ ID | CCAAUAUGUG |
| | | | | NO: | NO: | GCCGCCAGTA | NO: | NO: | GCCGCCAGUA |
| | | | | 2080] | 2081] | ATTTAAATGT | 2083] | 2084] | AUUUAAAUGU |
| | | | | | | CCATACAATT | | | CCAUACAAUU |
| | | | | | | ACTGTAGGCA | | | ACUGUAGGCA |
| | | | | | | ACATATTGCG | | | ACAUAUUGCG |
| | | | | | | TGAATGGGGC | | | UGAAUGGGGC |
| | | | | | | TGGCTGAGCA | | | UGGCUGAGCA |
| | | | | | | CCGTGGGTCG | | | CCGUGGGUCG |
| | | | | | | GCGAGGGCCC | | | GCGAGGGCCC |
| | | | | | | GCCAAGGA | | | GCCAAGGA |
| | | | | | | [SEQ ID | | | [SEQ ID |
| | | | | | | NO: | | | NO: |
| | | | | | | 2082] | | | 2085] |
| miR- | 3302 | −2.62809 | 0.136636 | TTGAACAAGG | CGCAAATCAG | GCAGGGCCGG | UUGAACAAGG | CGCAAAUCAG | GCAGGGCCGG |
| 130a | | | | GGCTGATTTG | ACCCTTGTTC | CATGCCTCTG | GGCUGAUUUG | ACCCUUGUUC | CAUGCCUCUG |
| | | | | GG | AC | CTGCTGGCCA | GG | AC | CUGCUGGCCA |
| | | | | [SEQ ID | [SEQ ID | CGCAAATCAG | [SEQ ID | [SEQ ID | CGCAAAUCAG |
| | | | | NO: | NO: | ACCCTTGTTC | NO: | NO: | ACCCUUGUUC |
| | | | | 688] | 1644] | ACCTGTCTGC | 1216] | 1839] | ACCUGUCUGC |
| | | | | | | ACCTGTCACT | | | ACCUGUCACU |
| | | | | | | AGTTGAACAA | | | AGUUGAACAA |
| | | | | | | GGGGCTGATT | | | GGGGCUGAUU |
| | | | | | | TGGGTGGCCG | | | UGGGUGGCCG |
| | | | | | | TGTAGTGCTA | | | UGUAGUGCUA |
| | | | | | | CCCAGCGCTG | | | CCCAGCGCUG |
| | | | | | | GCTGCCTCCT | | | GCUGCCUCCU |
| | | | | | | CAGCATTG | | | CAGCAUUG |
| | | | | | | [SEQ ID | | | [SEQ ID |
| | | | | | | NO: | | | NO: |
| | | | | | | 1722] | | | 1919] |
| miR- | 1755 | −2.56989 | 0.011195 | TCGGGTTGAA | CTCACTTCAG | GCAGGGCCGG | UCGGGUUGAA | CUCACUUCAG | GCAGGGCCGG |
| 130a | | | | ATCTGAAGTG | CTTTCAATTC | CATGCCTCTG | AUCUGAAGUG | CUUUCAAUUC | CAUGCCUCUG |
| | | | | TG | GC | CTGCTGGCCA | UG | GC | CUGCUGGCCA |
| | | | | [SEQ ID | [SEQ ID | CTCACTTCAG | [SEQ ID | [SEQ ID | CUCACUUCAG |
| | | | | NO: | NO: | CTTTCAATTC | NO: | NO: | CUUUCAAUUC |
| | | | | 657] | 1648] | GCCTGTCTGC | 1185] | 1843] | GCCUGUCUGC |
| | | | | | | ACCTGTCACT | | | ACCUGUCACU |
| | | | | | | AGTCGGGTTG | | | AGUCGGGUUG |
| | | | | | | AAATCTGAAG | | | AAAUCUGAAG |
| | | | | | | TGTGTGGCCG | | | UGUGUGGCCG |
| | | | | | | TGTAGTGCTA | | | UGUAGUGCUA |
| | | | | | | CCCAGCGCTG | | | CCCAGCGCUG |
| | | | | | | GCTGCCTCCT | | | GCUGCCUCCU |
| | | | | | | CAGCATTG | | | CAGCAUUG |
| | | | | | | [SEQ ID | | | [SEQ ID |
| | | | | | | NO: | | | NO: |
| | | | | | | 1728] | | | 1925] |
| miR- | 3301 | −2.55451 | −0.07529 | TGAACAAGGG | TGCCAAATCA | GCAGGGCCGG | UGAACAAGGG | UGCCAAAUCA | GCAGGGCCGG |
| 130a | | | | GCTGATTTGG | TCCCCTTGTT | CATGCCTCTG | GCUGAUUUGG | UCCCCUUGUU | CAUGCCUCUG |
| | | | | GA | CC | CTGCTGGCCA | GA | CC | CUGCUGGCCA |
| | | | | [SEQ ID | [SEQ ID | TGCCAAATCA | [SEQ ID | [SEQ ID | UGCCAAAUCA |
| | | | | NO: | NO: | TCCCCTTGTT | NO: | NO: | UCCCCUUGUU |
| | | | | 687] | 1649] | CCCTGTCTGC | 1215] | 1844] | CCCUGUCUGC |
| | | | | | | ACCTGTCACT | | | ACCUGUCACU |
| | | | | | | AGTGAACAAG | | | AGUGAACAAG |
| | | | | | | GGGCTGATTT | | | GGGCUGAUUU |
| | | | | | | GGGATGGCCG | | | GGGAUGGCCG |
| | | | | | | TGTAGTGCTA | | | UGUAGUGCUA |
| | | | | | | CCCAGCGCTG | | | CCCAGCGCUG |
| | | | | | | GCTGCCTCCT | | | GCUGCCUCCU |
| | | | | | | CAGCATTG | | | CAGCAUUG |
| | | | | | | [SEQ ID | | | [SEQ ID |
| | | | | | | NO: | | | NO: |
| | | | | | | 1730] | | | 1927] |
| miR- | 3341 | −2.43802 | 0.12379 | ATAGACATGA | GACTCAGCAT | GCAGGGCCGG | AUAGACAUGA | GACUCAGCAU | GCAGGGCCGG |
| 130a | | | | GGATGCTGAG | ACTCATGTTT | CATGCCTCTG | GGAUGCUGAG | ACUCAUGUUU | CAUGCCUCUG |
| | | | | AC | AC | CTGCTGGCCA | AC | AC | CUGCUGGCCA |
| | | | | [SEQ ID | [SEQ ID | GACTCAGCAT | [SEQ ID | [SEQ ID | GACUCAGCAU |
| | | | | NO: | NO: | ACTCATGTTT | NO: | NO: | ACUCAUGUUU |
| | | | | 1617] | 1667] | ACCTGTCTGC | 1813] | 1862] | ACCUGUCUGC |
| | | | | | | ACCTGTCACT | | | ACCUGUCACU |
| | | | | | | AGATAGACAT | | | AGAUAGACAU |
| | | | | | | GAGGATGCTG | | | GAGGAUGCUG |
| | | | | | | AGACTGGCCG | | | AGACUGGCCG |
| | | | | | | TGTAGTGCTA | | | UGUAGUGCUA |
| | | | | | | CCCAGCGCTG | | | CCCAGCGCUG |
| | | | | | | GCTGCCTCCT | | | GCUGCCUCCU |
| | | | | | | CAGCATTG | | | CAGCAUUG |
| | | | | | | [SEQ ID | | | [SEQ ID |
| | | | | | | NO: | | | NO: |
| | | | | | | 1753] | | | 1950] |
| miR- | 3330 | −2.43606 | 0.221874 | TATGCTGAGA | CGACATTATC | GCAGGGCCGG | UAUGCUGAGA | CGACAUUAUC | GCAGGGCCGG |
| 130a | | | | CTGATAATGT | CGTCTCAGTA | CATGCCTCTG | CUGAUAAUGU | CGUCUCAGUA | CAUGCCUCUG |
| | | | | GG | TC | CTGCTGGCCA | GG | UC | CUGCUGGCCA |
| | | | | [SEQ ID | [SEQ ID | CGACATTATC | [SEQ ID | [SEQ ID | CGACAUUAUC |
| | | | | NO: | NO: | CGTCTCAGTA | NO: | NO: | CGUCUCAGUA |
| | | | | 1614] | 1668] | TCCTGTCTGC | 1811] | 1863] | UCCUGUCUGC |
| | | | | | | ACCTGTCACT | | | ACCUGUCACU |
| | | | | | | AGTATGCTGA | | | AGUAUGCUGA |
| | | | | | | GACTGATAAT | | | GACUGAUAAU |
| | | | | | | GTGGTGGCCG | | | GUGGUGGCCG |
| | | | | | | TGTAGTGCTA | | | UGUAGUGCUA |
| | | | | | | CCCAGCGCTG | | | CCCAGCGCUG |
| | | | | | | GCTGCCTCCT | | | GCUGCCUCCU |
| | | | | | | CAGCATTG | | | CAGCAUUG |
| | | | | | | [SEQ ID | | | [SEQ ID |
| | | | | | | NO: | | | NO: |
| | | | | | | 1754] | | | 1951] |
| miR- | 3255 | −2.43262 | 0.01754 | ATTGCGTGGA | CGACCAGCTT | GCAGGGCCGG | AUUGCGUGGA | CGACCAGCUU | GCAGGGCCGG |
| 130a | | | | GTAAGCTGGT | CCTCCACGTA | CATGCCTCTG | GUAAGCUGGU | CCUCCACGUA | CAUGCCUCUG |
| | | | | GG | AC | CTGCTGGCCA | GG | AC | CUGCUGGCCA |
| | | | | [SEQ ID | [SEQ ID | CGACCAGCTT | [SEQ ID | [SEQ ID | CGACCAGCUU |
| | | | | NO: | NO: | CCTCCACGTA | NO: | NO: | CCUCCACGUA |
| | | | | 617] | 1669] | ACCTGTCTGC | 306] | 1864] | ACCUGUCUGC |
| | | | | | | ACCTGTCACT | (Same | | ACCUGUCACU |
| | | | | | | AGATTGCGTG | guide | | AGAUUGCGUG |
| | | | | | | GAGTAAGCTG | as | | GAGUAAGCUG |
| | | | | | | GTGGTGGCCG | XD- | | GUGGUGGCCG |
| | | | | | | TGTAGTGCTA | 14889) | | UGUAGUGCUA |
| | | | | | | CCCAGCGCTG | | | CCCAGCGCUG |
| | | | | | | GCTGCCTCCT | | | GCUGCCUCCU |
| | | | | | | CAGCATTG | | | CAGCAUUG |
| | | | | | | [SEQ ID | | | [SEQ ID |
| | | | | | | NO: | | | NO: |
| | | | | | | 1755] | | | 1952] |
| miR- | 3133 | −2.42664 | 0.412413 | TATGTCTTGG | CTGTGAATCA | GCAGGGCCGG | UAUGUCUUGG | CUGUGAAUCA | GCAGGGCCGG |
| 130a | | | | CTTGATTCAC | CGCCAAGATA | CATGCCTCTG | CUUGAUUCAC | CGCCAAGAUA | CAUGCCUCUG |
| | | | | TG | TC | CTGCTGGCCA | UG | UC | CUGCUGGCCA |
| | | | | [SEQ ID | [SEQ ID | CTGTGAATCA | [SEQ ID | [SEQ ID | CUGUGAAUCA |
| | | | | NO: | NO: | CGCCAAGATA | NO: | NO: | CGCCAAGAUA |
| | | | | 1624] | 1671] | TCCTGTCTGC | 1819] | 1866] | UCCUGUCUGC |
| | | | | | | ACCTGTCACT | | | ACCUGUCACU |
| | | | | | | AGTATGTCTT | | | AGUAUGUCUU |
| | | | | | | GGCTTGATTC | | | GGCUUGAUUC |
| | | | | | | ACTGTGGCCG | | | ACUGUGGCCG |
| | | | | | | TGTAGTGCTA | | | UGUAGUGCUA |
| | | | | | | CCCAGCGCTG | | | CCCAGCGCUG |
| | | | | | | GCTGCCTCCT | | | GCUGCCUCCU |
| | | | | | | CAGCATTG | | | CAGCAUUG |
| | | | | | | [SEQ ID | | | [SEQ ID |
| | | | | | | NO: | | | NO: |
| | | | | | | 1757] | | | 1954] |
| miR- | 3842 | −2.39495 | −0.55009 | AACGTGAGAA | TTCGATCCAT | GCAGGGCCGG | AACGUGAGAA | UUCGAUCCAU | GCAGGGCCGG |
| 130a | | | | GGATGGATCG | ACTTCTCATG | CATGCCTCTG | GGAUGGAUCG | ACUUCUCAUG | CAUGCCUCUG |
| | | | | TA | TC | CTGCTGGCCA | UA | UC | CUGCUGGCCA |
| | | | | [SEQ ID | [SEQ ID | TTCGATCCAT | [SEQ ID | [SEQ ID | UUCGAUCCAU |
| | | | | NO: | NO: | ACTTCTCATG | NO: | NO: | ACUUCUCAUG |
| | | | | 1625] | 1677] | T | 1824] | 1872] | UC |
| | | | | | | CCTGTCTGCA | | | CUGUCUGCAC |
| | | | | | | CCTGTCACTA | | | CUGUCACUAG |
| | | | | | | GAACGTGAGA | | | AACGUGAGAA |
| | | | | | | AGGATGGATC | | | GGAUGGAUCG |
| | | | | | | GTATGGCCGT | | | UAUGGCCGUG |
| | | | | | | GTAGTGCTAC | | | UAGUGCUACC |
| | | | | | | CCAGCGCTGG | | | CAGCGCUGGC |
| | | | | | | CTGCCTCCTC | | | UGCCUCCUCA |
| | | | | | | AGCATTG | | | GCAUUG |
| | | | | | | [SEQ ID | | | [SEQ ID |
| | | | | | | NO: | | | NO: |
| | | | | | | 1767] | | | 1964] |
| miR- | 2586 | −2.38521 | 0.042149 | TAGATTCAGA | CGAAGTTCTA | GCAGGGCCGG | UAGAUUCAGA | CGAAGUUCUA | GCAGGGCCGG |
| 130a | | | | AGTAGAACTT | ATTCTGAATC | CATGCCTCTG | AGUAGAACUU | AUUCUGAAUC | CAUGCCUCUG |
| | | | | GG | TC | CTGCTGGCCA | GG | UC | CUGCUGGCCA |
| | | | | [SEQ ID | [SEQ ID | CGAAGTTCTA | [SEQ ID | [SEQ ID | CGAAGUUCUA |
| | | | | NO: | NO: | ATTCTGAATC | NO: | NO: | AUUCUGAAUC |
| | | | | 1621] | 1679] | TCCTGTCTGC | 1816] | 1874] | UCCUGUCUGC |
| | | | | | | ACCTGTCACT | | | ACCUGUCACU |
| | | | | | | AGTAGATTCA | | | AGUAGAUUCA |
| | | | | | | GAAGTAGAAC | | | GAAGUAGAAC |
| | | | | | | TTGGTGGCCG | | | UUGGUGGCCG |
| | | | | | | TGTAGTGCTA | | | UGUAGUGCUA |
| | | | | | | CCCAGCGCTG | | | CCCAGCGCUG |
| | | | | | | GCTGCCTCCT | | | GCUGCCUCCU |
| | | | | | | CAGCATTG | | | CAGCAUUG |
| | | | | | | [SEQ ID | | | [SEQ ID |
| | | | | | | NO: | | | NO: |
| | | | | | | 1769] | | | 1966] |
| miR- | 2602 | −2.30701 | −0.14983 | TTTAGTAGTT | ATTCTATGGA | GCAGGGCCGG | UUUAGUAGUU | AUUCUAUGGA | GCAGGGCCGG |
| 130a | | | | GATCCATAGA | GCAACTATTA | CATGCCTCTG | GAUCCAUAGA | GCAACUAUUA | CAUGCCUCUG |
| | | | | TT | AC | CTGCTGGCCA | UU | AC | CUGCUGGCCA |
| | | | | [SEQ ID | [SEQ ID | ATTCTATGGA | [SEQ ID | [SEQ ID | AUUCUAUGGA |
| | | | | NO: | NO: | GCAACTATTA | NO: | NO: | GCAACUAUUA |
| | | | | 1616] | 1696] | ACCTGTCTGC | 202] | 1891] | ACCUGUCUGC |
| | | | | | | ACCTGTCACT | (Same | | ACCUGUCACU |
| | | | | | | AGTTTAGTAG | guide | | AGUUUAGUAG |
| | | | | | | TTGATCCATA | as | | UUGAUCCAUA |
| | | | | | | GATTTGGCCG | XD- | | GAUUUGGCCG |
| | | | | | | TGTAGTGCTA | 14837) | | UGUAGUGCUA |
| | | | | | | CCCAGCGCTG | | | CCCAGCGCUG |
| | | | | | | GCTGCCTCCT | | | GCUGCCUCCU |
| | | | | | | CAGCATTG | | | CAGCAUUG |
| | | | | | | [SEQ ID | | | [SEQ ID |
| | | | | | | NO: | | | NO: |
| | | | | | | 1789] | | | 1986] |
| miR- | 3341 | −2.48196 | 0.14613 | ATAGACATGA | GCCTCAGCAT | GCCGTCCGCG | AUAGACAUGA | GCCUCAGCAU | GCCGUCCGCG |
| 132 | | | | GGATGCTGAG | CATAATGTCT | CGCCCCGCCC | GGAUGCUGAG | CAUAAUGUCU | CGCCCCGCCC |
| | | | | AC | AT | CCGCGTCTCC | AC | AU | CCGCGUCUCC |
| | | | | [SEQ ID | [SEQ ID | AGGGGCCTCA | [SEQ ID | [SEQ ID | AGGGGCCUCA |
| | | | | NO: | NO: | GCATCATAAT | NO: | NO: | GCAUCAUAAU |
| | | | | 1617] | 1658] | GTCTATCTGT | 1813] | 1853] | GUCUAUCUGU |
| | | | | | | GGGAACTGGA | | | GGGAACUGGA |
| | | | | | | GGATAGACAT | | | GGAUAGACAU |
| | | | | | | GAGGA | | | GAGGAUG |
| | | | | | | TGCTGAGACC | | | CUGAGACCCC |
| | | | | | | CCCGCAGCAC | | | CGCAGCACGC |
| | | | | | | GCCCACGCGC | | | CCACGCGCCG |
| | | | | | | CGCGCCACGC | | | CGCCACGCCG |
| | | | | | | CGCGCCCCGA | | | CGCCCCGAGC |
| | | | | | | GCC | | | C |
| | | | | | | [SEQ ID | | | [SEQ ID |
| | | | | | | NO: | | | NO: |
| | | | | | | 1742] | | | 1939] |
| miR- | 3302 | −2.43028 | 0.018802 | TTGAACAAGG | CACAAATCAG | GCCGTCCGCG | UUGAACAAGG | CACAAAUCAG | GCCGUCCGCG |
| 132 | | | | GGCTGATTTG | CACATTGTTC | CGCCCCGCCC | GGCUGAUUUG | CACAUUGUUC | CGCCCCGCCC |
| | | | | GG | AA | CCGCGTCTCC | GG | AA | CCGCGUCUCC |
| | | | | [SEQ ID | [SEQ ID | AGGGCACAAA | [SEQ ID | [SEQ ID | AGGGCACAAA |
| | | | | NO: | NO: | TCAGCACATT | NO: | NO: | UCAGCACAUU |
| | | | | 688] | 1670] | GTTCAACTGT | 1216] | 1865] | GUUCAACUGU |
| | | | | | | GGGAACTGGA | | | GGGAACUGGA |
| | | | | | | GGTTGAACAA | | | GGUUGAACAA |
| | | | | | | GGGGCTGATT | | | GGGGCUGAUU |
| | | | | | | TGGGCCCCGC | | | UGGGCCCCGC |
| | | | | | | AGCACGCCCA | | | AGCACGCCCA |
| | | | | | | CGCGCCGCGC | | | CGCGCCGCGC |
| | | | | | | CACGCCGCGC | | | CACGCCGCGC |
| | | | | | | CCCGAGCC | | | CCCGAGCC |
| | | | | | | [SEQ ID | | | [SEQ ID |
| | | | | | | NO: | | | NO: |
| | | | | | | 1756] | | | 1953] |
| miR- | 1755 | −2.41844 | 0.196175 | TCGGGTTGAA | CCCACTTCAG | GCCGTCCGCG | UCGGGUUGAA | CCCACUUCAG | GCCGUCCGCG |
| 132 | | | | ATCTGAAGTG | AGTGCAACCC | CGCCCCGCCC | AUCUGAAGUG | AGUGCAACCC | CGCCCCGCCC |
| | | | | TG | GA | CCGCGTCTCC | UG | GA | CCGCGUCUCC |
| | | | | [SEQ ID | [SEQ ID | AGGGCCCACT | [SEQ ID | [SEQ ID | AGGGCCCACU |
| | | | | NO: | NO: | TCAGAGTGCA | NO: | NO: | UCAGAGUGCA |
| | | | | 657] | 1672] | ACCCGACTGT | 1185] | 1867] | ACCCGACUGU |
| | | | | | | GGGAACTGGA | | | GGGAACUGGA |
| | | | | | | GGTCGGGTTG | | | GGUCGGGUUG |
| | | | | | | AAATCTGAAG | | | AAAUCUGAAG |
| | | | | | | TGTGCCCCGC | | | UGUGCCCCGC |
| | | | | | | AGCACGCCCA | | | AGCACGCCCA |
| | | | | | | CGCGCCGCGC | | | CGCGCCGCGC |
| | | | | | | CACGCCGCGC | | | CACGCCGCGC |
| | | | | | | CCCGAGCC | | | CCCGAGCC |
| | | | | | | [SEQ ID | | | [SEQ ID |
| | | | | | | NO: | | | NO: |
| | | | | | | 1762] | | | 1959] |
| miR- | 2602 | −2.40987 | −0.18915 | TTTAGTAGTT | ACTCTATGGA | GCCGTCCGCG | UUUAGUAGUU | ACUCUAUGGA | GCCGUCCGCG |
| 132 | | | | GATCCATAGA | TAACCTACTA | CGCCCCGCCC | GAUCCAUAGA | UAACCUACUA | CGCCCCGCCC |
| | | | | TT | AA | CCGCGTCTCC | UU | AA | CCGCGUCUCC |
| | | | | [SEQ ID | [SEQ ID | AGGGACTCTA | [SEQ ID | [SEQ ID | AGGGACUCUA |
| | | | | NO: | NO: | TGGATAACCT | NO: | NO: | UGGAUAACCU |
| | | | | 1616] | 1674] | ACTAAACTGT | 202] | 1869] | ACUAAACUGU |
| | | | | | | GGGAACTGGA | (Same | | GGGAACUGGA |
| | | | | | | GGTTTAGTAG | guide | | GGUUUAGUAG |
| | | | | | | TTGATCCATA | as | | UUGAUCCAUA |
| | | | | | | GATTCCCCG | XD- | | GAUUCCCCGC |
| | | | | | | CAGCACGCCC | 14837) | | A |
| | | | | | | ACGCGCCGCG | | | GCACGCCCAC |
| | | | | | | CCACGCCGCG | | | GCGCCGCGCC |
| | | | | | | CCCCGAGCC | | | ACGCCGCGCC |
| | | | | | | [SEQ ID | | | CCGAGCC |
| | | | | | | NO: | | | [SEQ ID |
| | | | | | | 1764] | | | NO: |
| | | | | | | | | | 1961] |
| miR- | 1784 | −2.34143 | −0.26117 | ATTAACTACT | TCCAGACCAA | GCCGTCCGCG | AUUAACUACU | UCCAGACCAA | GCCGUCCGCG |
| 132 | | | | CTTTGGTCTG | ATATTAGTTA | CGCCCCGCCC | CUUUGGUCUG | AUAUUAGUUA | CGCCCCGCCC |
| | | | | AA | AT | CCGCGTCTCC | AA | AU | CCGCGUCUCC |
| | | | | [SEQ ID | [SEQ ID | AGGGTCCAGA | [SEQ ID | [SEQ ID | AGGGUCCAGA |
| | | | | NO: | NO: | CCAAATATTA | NO: | NO: | CCAAAUAUUA |
| | | | | 608] | 1686] | GTTAATCTGT | 112] | 1881] | GUUAAUCUGU |
| | | | | | | GGGAACTGGA | (Same | | GGGAACUGGA |
| | | | | | | GGATTAACTA | guide | | GGAUUAACUA |
| | | | | | | CTCTTTGGTC | as | | CUCUUUGGUC |
| | | | | | | TGAACCCCGC | XD- | | UGAACCCCGC |
| | | | | | | AGCACGCCCA | 14792) | | AGCACGCCCA |
| | | | | | | CGCGCCGCGC | | | CGCGCCGCGC |
| | | | | | | CACGCCGCGC | | | CACGCCGCGC |
| | | | | | | CCCGAGCC | | | CCCGAGCC |
| | | | | | | [SEQ ID | | | [SEQ ID |
| | | | | | | NO: | | | NO: |
| | | | | | | 1779] | | | 1976] |
| miR- | 3338 | −2.2984 | −0.03531 | TACATGAGGA | TAAGTCTCAG | GCCGTCCGCG | UACAUGAGGA | UAAGUCUCAG | GCCGUCCGCG |
| 132 | | | | TGCTGAGACT | CCTACTCATG | CGCCCCGCCC | UGCUGAGACU | CCUACUCAUG | CGCCCCGCCC |
| | | | | GA | TA | CCGCGTCTCC | GA | UA | CCGCGUCUCC |
| | | | | [SEQ ID | [SEQ ID | AGGGTAAGTC | [SEQ ID | [SEQ ID | AGGGUAAGUC |
| | | | | NO: | NO: | TCAGCCTACT | NO: | NO: | UCAGCCUACU |
| | | | | 1620] | 1698] | CATGTACTGT | 314] | 1893] | CAUGUACUGU |
| | | | | | | GGGAACTGGA | (Same | | GGGAACUGGA |
| | | | | | | GGTACATGAG | guide | | GGUACAUGAG |
| | | | | | | GATGCTGAGA | as | | GAUGCUGAGA |
| | | | | | | CTGACCCCGC | XD- | | CUGACCCCGC |
| | | | | | | AGCACGCCCA | 14893) | | AGCACGCCCA |
| | | | | | | CGCGCCGCGC | | | CGCGCCGCGC |
| | | | | | | CACGCCGCGC | | | CACGCCGCGC |
| | | | | | | CCCGAGCC | | | CCCGAGCC |
| | | | | | | [SEQ ID | | | [SEQ ID |
| | | | | | | NO: | | | NO: |
| | | | | | | 1791] | | | 1988] |
| miR- | 2945 | −2.18195 | 0.054975 | TGTAGTAGAA | TAAGCCAAAG | GCCGTCCGCG | UGUAGUAGAA | UAAGCCAAAG | GCCGUCCGCG |
| 132 | | | | GGCTTTGGCT | CATGCTACTA | CGCCCCGCCC | GGCUUUGGCU | CAUGCUACUA | CGCCCCGCCC |
| | | | | GA | CA | CCGCGTCTCC | GA | CA | CCGCGUCUCC |
| | | | | [SEQ ID | [SEQ ID | AGGGTAAGCC | [SEQ ID | [SEQ ID | AGGGUAAGCC |
| | | | | NO: | NO: | AAAGCATGCT | NO: | NO: | AAAGCAUGCU |
| | | | | 685] | 2086] | ACTACACTGT | 1213] | 2088] | ACUACACUGU |
| | | | | | | GGGAACTGGA | (Same | | GGGAACUGGA |
| | | | | | | GGTGTAGTAG | guide | | GGUGUAGUAG |
| | | | | | | AAGGCTTTGG | as | | AAGGCUUUGG |
| | | | | | | CTGACCCCGC | XD- | | CUGACCCCGC |
| | | | | | | AGCACGCCCA | 14860) | | AGCACGCCCA |
| | | | | | | | | | CGCG |
| | | | | | | CGCGCCGCGC | | | CCGCGCCACG |
| | | | | | | CACGCCGCGC | | | CCGCGCCCCG |
| | | | | | | CCCGAGCC | | | AGCC |
| | | | | | | [SEQ ID | | | [SEQ ID |
| | | | | | | NO: | | | NO: |
| | | | | | | 2087] | | | 2089] |
| miR- | 3256 | −2.15429 | 0.256242 | TATTGCGTGG | CCCCAGCTTA | GCCGTCCGCG | UAUUGCGUGG | CCCCAGCUUA | GCCGUCCGCG |
| 132 | | | | AGTAAGCTGG | CGCAACGCAA | CGCCCCGCCC | AGUAAGCUGG | CGCAACGCAA | CGCCCCGCCC |
| | | | | TG | TA | CCGCGTCTCC | UG | UA | CCGCGUCUCC |
| | | | | [SEQ ID | [SEQ ID | AGGGCCCCAG | [SEQ ID | [SEQ ID | AGGGCCCCAG |
| | | | | NO: | NO: | CTTACGCAAC | NO: | NO: | CUUACGCAAC |
| | | | | 618] | 2090] | GCAATACTGT | 308] | 2092] | GCAAUACUGU |
| | | | | | | GGGAACTGGA | (Same | | GGGAACUGGA |
| | | | | | | GGTATTGCGT | as | | GGUAUUGCGU |
| | | | | | | GGAGTAAGCT | XD- | | GGAGUAAGCU |
| | | | | | | GGTGCCCCGC | 14890) | | GGUGCCCCGC |
| | | | | | | AGCACGCCCA | | | AGCACGCCCA |
| | | | | | | CGCGCCGCGC | | | CGCGCCGCGC |
| | | | | | | CACGCCGCGC | | | CACGCCGCGC |
| | | | | | | CCCGAGCC | | | CCCGAGCC |
| | | | | | | [SEQ ID | | | [SEQ ID |
| | | | | | | NO: | | | NO: |
| | | | | | | 2091] | | | 2093] |
| miR- | 3255 | −2.09429 | 0.188946 | ATTGCGTGGA | CAACCAGCTT | GCCGTCCGCG | AUUGCGUGGA | CAACCAGCUU | GCCGUCCGCG |
| 132 | | | | GTAAGCTGGT | AATACACGCA | CGCCCCGCCC | GUAAGCUGGU | AAUACACGCA | CGCCCCGCCC |
| | | | | GG | AT | CCGCGTCTCC | GG | AU | CCGCGUCUCC |
| | | | | [SEQ ID | [SEQ ID | AGGGCAACCA | [SEQ ID | [SEQ ID | AGGGCAACCA |
| | | | | NO: | NO: | GCTTAATACA | NO: | NO: | GCUUAAUACA |
| | | | | 617] | 2094] | CGCAATCTGT | 306] | 2096] | CGCAAUCUGU |
| | | | | | | GGGAACTGGA | (Same | | GGGAACUGGA |
| | | | | | | GGATTGCGTG | guide | | GGAUUGCGUG |
| | | | | | | GAGTAAGCTG | as | | GAGUAAGCUG |
| | | | | | | GTGGCCCCGC | XD- | | GUGGCCCCGC |
| | | | | | | AGCACGCCCA | 14889) | | AGCACGCCCA |
| | | | | | | CGCGCCGCGC | | | CGCGCCGCGC |
| | | | | | | CACGCCGCGC | | | CACGCCGCGC |
| | | | | | | CCCGAGCC | | | CCCGAGCC |
| | | | | | | [SEQ ID | | | [SEQ ID |
| | | | | | | NO: | | | NO: |
| | | | | | | 2095] | | | 2097] |
| miR- | 2928 | −2.08992 | 0.223976 | TCTGAGAGAA | ACCCCACGTT | GCCGTCCGCG | UCUGAGAGAA | ACCCCACGUU | GCCGUCCGCG |
| 132 | | | | GGAACGTGGG | CATGCTCTCA | CGCCCCGCCC | GGAACGUGGG | CAUGCUCUCA | CGCCCCGCCC |
| | | | | TT | GA | CCGCGTCTCC | UU | GA | CCGCGUCUCC |
| | | | | [SEQ ID | [SEQ ID | AGGGACCCCA | [SEQ ID | [SEQ ID | AGGGACCCCA |
| | | | | NO: | NO: | CGTTCATGCT | NO: | NO: | CGUUCAUGCU |
| | | | | 668] | 2098] | CTCAGACTGT | 1196] | 2100] | CUCAGACUGU |
| | | | | | | GGGAACTGGA | | | GGGAACUGGA |
| | | | | | | GGTCTGAGAG | | | GGUCUGAGAG |
| | | | | | | AAGGAACGTG | | | AAGGAACGUG |
| | | | | | | GGTTCCCCGC | | | GGUUCCCCGC |
| | | | | | | AGCACGCCCA | | | AGCACGCCCA |
| | | | | | | CGCGCCGCGC | | | CGCG |
| | | | | | | CAC | | | CCGCGCCACG |
| | | | | | | GCCGCGCCCC | | | CCGCGCCCCG |
| | | | | | | GAGCC | | | AGCC |
| | | | | | | [SEQ ID | | | [SEQ ID |
| | | | | | | NO: | | | NO: |
| | | | | | | 2099] | | | 2101] |
| miR- | 3338 | −2.01915 | −0.127 | TACATGAGGA | TCAGAACAGC | GCCGGCGGAG | UACAUGAGGA | UCAGAACAGC | GCCGGCGGAG |
| 138-2 | | | | TGCTGAGACT | ATCCTCATGA | TTCTGGTATC | UGCUGAGACU | AUCCUCAUGA | UUCUGGUAUC |
| | | | | GA | [SEQ ID | GTTGCTGCTA | GA | [SEQ ID | GUUGCUGCUA |
| | | | | [SEQ ID | NO: | CATGAGGATG | [SEQ ID | NO: | CAUGAGGAUG |
| | | | | NO: | 2102] | CTGAGACTGA | NO: | 2104] | CUGAGACUGA |
| | | | | 1620] | | GACGAGCAGC | 314] | | GACGAGCAGC |
| | | | | | | GCATCCTCTT | (Same | | GCAUCCUCUU |
| | | | | | | ACCCTCAGAA | guide | | ACCCUCAGAA |
| | | | | | | CAGCATCCTC | as | | CAGCAUCCUC |
| | | | | | | ATGAGTTGCA | XD- | | AUGAGUUGCA |
| | | | | | | TCATACCCAT | 14893) | | UCAUACCCAU |
| | | | | | | CCTCTCCAGG | | | CCUCUCCAGG |
| | | | | | | CGAGCCTCGT | | | CGAGCCUCGU |
| | | | | | | GGGACCGG | | | GGGACCGG |
| | | | | | | [SEQ ID | | | [SEQ ID |
| | | | | | | NO: | | | NO: |
| | | | | | | 2103] | | | 2105] |
| miR- | 3330 | −1.83218 | 0.060443 | TATGCTGAGA | CCACTGATCA | GCCGGCGGAG | UAUGCUGAGA | CCACUGAUCA | GCCGGCGGAG |
| 138-2 | | | | CTGATAATGT | GTCTCAGCAA | TTCTGGTATC | CUGAUAAUGU | GUCUCAGCAA | UUCUGGUAUC |
| | | | | GG | [SEQ ID | GTTGCTGCTA | GG | [SEQ ID | GUUGCUGCUA |
| | | | | [SEQ ID | NO: | TGCTGAGACT | [SEQ ID | NO: | UGCUGAGACU |
| | | | | NO: | 2106] | GATAATGTGG | NO: | 2108] | GAUAAUGUGG |
| | | | | 1614] | | GACGAGCAGC | 1811] | | GACGAGCAGC |
| | | | | | | GCATCCTCTT | | | GCAUCCUCUU |
| | | | | | | ACCCCCACTG | | | ACCCCCACUG |
| | | | | | | ATCAGTCTCA | | | AUCAGUCUCA |
| | | | | | | GCAAGTTGCA | | | GCAAGUUGCA |
| | | | | | | TCATACCCAT | | | UCAUACCCAU |
| | | | | | | CCTCTCCAGG | | | CCUCUCCAGG |
| | | | | | | CGAGCCTCGT | | | CGAGCCUCGU |
| | | | | | | GGGACCGG | | | GGGACCGG |
| | | | | | | [SEQ ID | | | [SEQ ID |
| | | | | | | NO: | | | NO: |
| | | | | | | 2107] | | | 2109] |
| miR- | 3043 | −1.6949 | 0.083253 | TTTGGTGCAA | AGCCATTTGT | GCCGGCGGAG | UUUGGUGCAA | AGCCAUUUGU | GCCGGCGGAG |
| 138-2 | | | | AACAAACAGG | TTTGCACCAT | TTCTGGTATC | AACAAACAGG | UUUGCACCAU | UUCUGGUAUC |
| | | | | CT | [SEQ ID | GTTGCTGCTT | CU | [SEQ ID | GUUGCUGCUU |
| | | | | [SEQ ID | NO: | TGGTGCAAAA | [SEQ ID | NO: | UGGUGCAAAA |
| | | | | NO: | 2110] | CAAACAGGCT | NO: | 2112] | CAAACAGGCU |
| | | | | 1615] | | GACGAGCAGC | 1812] | | GACGAGCAGC |
| | | | | | | GCATCCTCTT | | | GCAUCCUCUU |
| | | | | | | ACCCAGCCAT | | | ACCCAGCCAU |
| | | | | | | TTGTTTTGCA | | | UUGUUUUGCA |
| | | | | | | CCATGTTGCA | | | CCAUGUUGCA |
| | | | | | | TCATACCCAT | | | UCAUACCCAU |
| | | | | | | CCTCTCCAGG | | | CCUCUCCAGG |
| | | | | | | CGAGCCTCGT | | | CGAGCCUCGU |
| | | | | | | GGGACCGG | | | GGGACCGG |
| | | | | | | [SEQ ID | | | [SEQ ID |
| | | | | | | NO: | | | NO: |
| | | | | | | 2111] | | | 2113] |
| miR- | 1689 | −1.57706 | −0.53719 | TACGCGGTGA | GGGACCAGAA | GCCGGCGGAG | UACGCGGUGA | GGGACCAGAA | GCCGGCGGAG |
| 138-2 | | | | ATTCTGTCTC | TTCACCGCGA | TTCTGGTATC | AUUCUGUCUC | UUCACCGCGA | UUCUGGUAUC |
| | | | | CC | [SEQ ID | GTTGCTGCTA | CC | [SEQ ID | GUUGCUGCUA |
| | | | | [SEQ ID | NO: | CGCGGTGAAT | [SEQ ID | NO: | CGCGGUGAAU |
| | | | | NO: | 2114] | TCTGTCTCCC | NO: | 2116] | UCUGUCUCCC |
| | | | | 605] | | GACGAGCAGC | 100] | | GACGAGCAGC |
| | | | | | | GCATCCTCTT | (Same | | GCAUCCUCUU |
| | | | | | | ACCCGGGACC | guide | | ACCCGGGACC |
| | | | | | | AGAATTCACC | as | | AGAAUUCACC |
| | | | | | | GCGAGTTGCA | XD- | | GCGAGUUGCA |
| | | | | | | TCATACCCAT | 14786) | | UCAUACCCAU |
| | | | | | | CCTCTCCAGG | | | CCUCUCCAGG |
| | | | | | | CGAGCCTCGT | | | CGAGCCUCGU |
| | | | | | | GGGACCGG | | | GGGACCGG |
| | | | | | | [SEQ ID | | | [SEQ ID |
| | | | | | | NO: | | | NO: |
| | | | | | | 2115] | | | 2117] |
| miR- | 3270 | −1.41498 | 0.087791 | TACTGTAGGC | ACGCTCATGT | GCCGGCGGAG | UACUGUAGGC | ACGCUCAUGU | GCCGGCGGAG |
| 138-2 | | | | AACATATTGC | TGCCTACAGA | TTCTGGTATC | AACAUAUUGC | UGCCUACAGA | UUCUGGUAUC |
| | | | | GT | [SEQ ID | GTTGCTGCTA | GU | [SEQ ID | GUUGCUGCUA |
| | | | | [SEQ ID | NO: | CTGTAGGCAA | [SEQ ID | NO: | CUGUAGGCAA |
| | | | | NO: | 1901] | CATATTGCGT | NO: | 2119] | CAUAUUGCGU |
| | | | | 2080] | | GACGAGCAGC | 2083] | | GACGAGCAGC |
| | | | | | | GCATCCTCTT | | | GCAUCCUCUU |
| | | | | | | ACCCACGCTC | | | ACCCACGCUC |
| | | | | | | ATGTTGCCTA | | | AUGUUGCCUA |
| | | | | | | CAGAGTTGCA | | | CAGAGUUGCA |
| | | | | | | TCATACCCAT | | | UCAUACCCAU |
| | | | | | | CCTCTCCAGG | | | CCUCUCCAGG |
| | | | | | | CGAGCCTCGT | | | CGAGCCUCGU |
| | | | | | | GGGACCGG | | | GGGACCGG |
| | | | | | | [SEQ ID | | | [SEQ ID |
| | | | | | | NO: | | | NO: |
| | | | | | | 2118] | | | 2120] |
| miR- | 2928 | −1.36141 | 0.122452 | TCTGAGAGAA | AACCGCGTTC | GCCGGCGGAG | UCUGAGAGAA | AACCGCGUUC | GCCGGCGGAG |
| 138-2 | | | | GGAACGTGGG | CTTCTCTCAC | TTCTGGTATC | GGAACGUGGG | CUUCUCUCAC | UUCUGGUAUC |
| | | | | TT | [SEQ ID | GTTGCTGCTC | UU | [SEQ ID | GUUGCUGCUC |
| | | | | [SEQ ID | NO: | TGAGAGAAGG | [SEQ ID | NO: | UGAGAGAAGG |
| | | | | NO: | 2121] | AACGTGGGTT | NO: | 2123] | AACGUGGGUU |
| | | | | 668] | | GACGAGCAGC | 1196] | | GACGAGCAGC |
| | | | | | | GCATCCTCTT | | | GCAUCCUCUU |
| | | | | | | ACCCAACCGC | | | ACCCAACCGC |
| | | | | | | GTTCCTTCTC | | | GUUCCUUCUC |
| | | | | | | TCACGTTGCA | | | UCACGUUGCA |
| | | | | | | TCATACCCAT | | | UCAUACCCAU |
| | | | | | | CCTCTCCAGG | | | CCUCUCCAGG |
| | | | | | | CGAGCCTCGT | | | CGAGCCUCGU |
| | | | | | | GGGACCGG | | | GGGACCGG |
| | | | | | | [SEQ ID | | | [SEQ ID |
| | | | | | | NO: | | | NO: |
| | | | | | | 2122] | | | 2124] |
| miR- | 3273 | −1.32764 | 0.033491 | TAGGACTGTA | CAATTGTTGC | GCCGGCGGAG | UAGGACUGUA | CAAUUGUUGC | GCCGGCGGAG |
| 138-2 | | | | GGCAACATAT | CTACAGTCCA | TTCTGGTATC | GGCAACAUAU | CUACAGUCCA | UUCUGGUAUC |
| | | | | TG | [SEQ ID | GTTGCTGCTA | UG | [SEQ ID | GUUGCUGCUA |
| | | | | [SEQ ID | NO: | GGACTGTAGG | [SEQ ID | NO: | GGACUGUAGG |
| | | | | NO: | 2125] | CAACATATTG | NO: | 2127] | CAACAUAUUG |
| | | | | 1628] | | GACGAGCAGC | 1821] | | GACGAGCAGC |
| | | | | | | GCATCCTCTT | | | GCAUCCUCUU |
| | | | | | | ACCCCAATTG | | | ACCCCAAUUG |
| | | | | | | TTGCCTACAG | | | UUGCCUACAG |
| | | | | | | TCCAGTTGCA | | | UCCAGUUGCA |
| | | | | | | TCATACCCAT | | | UCAUACCCAU |
| | | | | | | CCTCTCCAGG | | | CCUCUCCAGG |
| | | | | | | CGAGCCTCGT | | | CGAGCCUCGU |
| | | | | | | GGGACCGG | | | GGGACCGG |
| | | | | | | [SEQ ID | | | [SEQ ID |
| | | | | | | NO: | | | NO: |
| | | | | | | 2126] | | | 2128] |
| miR- | 3255 | −1.19435 | 0.020971 | ATTGCGTGGA | CCACGCCTTA | GCCGGCGGAG | AUUGCGUGGA | CCACGCCUUA | GCCGGCGGAG |
| 138-2 | | | | GTAAGCTGGT | CTCCACGCAT | TTCTGGTATC | GUAAGCUGGU | CUCCACGCAU | UUCUGGUAUC |
| | | | | GG | [SEQ ID | GTTGCTGCAT | GG | [SEQ ID | GUUGCUGCAU |
| | | | | [SEQ ID | NO: | TGCGTGGAGT | [SEQ ID | NO: | UGCGUGGAGU |
| | | | | NO: | 2129] | AAGCTGGTGG | NO: | 2131] | AAGCUGGUGG |
| | | | | 617] | | GACGAGCAGC | 306] | | GACGAGCAGC |
| | | | | | | GCATCCTCTT | (Same | | GCAUCCUCUU |
| | | | | | | ACCCCCACGC | guide | | ACCCCCACGC |
| | | | | | | CTTACTCCAC | as | | CUUACUCCAC |
| | | | | | | GCATGTTGCA | XD- | | GCAUGUUGCA |
| | | | | | | TCATACCCAT | 14889) | | UCAUACCCAU |
| | | | | | | CCTCTCCAGG | | | CCUCUCCAGG |
| | | | | | | CGAGCCTCGT | | | CGAGCCUCGU |
| | | | | | | GGGACCGG | | | GGGACCGG |
| | | | | | | [SEQ ID | | | [SEQ ID |
| | | | | | | NO: | | | NO: |
| | | | | | | 2130] | | | 2132] |
| miR- | 3302 | −1.02575 | −0.2011 | TTGAACAAGG | CCCATCCAGC | GCCGGCGGAG | UUGAACAAGG | CCCAUCCAGC | GCCGGCGGAG |
| 138-2 | | | | GGCTGATTTG | CTCTTGTTCT | TTCTGGTATC | GGCUGAUUUG | CUCUUGUUCU | UUCUGGUAUC |
| | | | | GG | [SEQ ID | GTTGCTGCTT | GG | [SEQ ID | GUUGCUGCUU |
| | | | | [SEQ ID | NO: | GAACAAGGGG | [SEQ ID | NO: | GAACAAGGGG |
| | | | | NO: | 2133] | CTGATTTGGG | NO: | 2135] | CUGAUUUGGG |
| | | | | 688] | | GACGAGCAGC | 1216] | | GACGAGCAGC |
| | | | | | | GCATCCTCTT | | | GCAUCCUCUU |
| | | | | | | ACCCCCCATC | | | ACCCCCCAUC |
| | | | | | | CAGCCTCTTG | | | CAGCCUCUUG |
| | | | | | | TTCTGTTGCA | | | UUCUGUUGCA |
| | | | | | | TCATACCCAT | | | UCAUACCCAU |
| | | | | | | CCTCTCCAGG | | | CCUCUCCAGG |
| | | | | | | CGAGCCTCGT | | | CGAGCCUCGU |
| | | | | | | GGGACCGG | | | GGGACCGG |
| | | | | | | [SEQ ID | | | [SEQ ID |
| | | | | | | NO: | | | NO: |
| | | | | | | 2134] | | | 2136] |
| miR- | 1436 | −0.97653 | 0.111912 | TGAGTTATCT | GCCCAGGAAA | GCCGGCGGAG | UGAGUUAUCU | GCCCAGGAAA | GCCGGCGGAG |
| 138-2 | | | | CTTTCTAAGG | GAGATAACTG | TTCTGGTATC | CUUUCUAAGG | GAGAUAACUG | UUCUGGUAUC |
| | | | | GC | [SEQ ID | GTTGCTGCTG | GC | [SEQ ID | GUUGCUGCUG |
| | | | | [SEQ ID | NO: | AGTTATCTCT | [SEQ ID | NO: | AGUUAUCUCU |
| | | | | NO: | 2137] | TTCTAAGGGC | NO: | 2139] | UUCUAAGGGC |
| | | | | 1632] | | GACGAGCAGC | 1827] | | GACGAGCAGC |
| | | | | | | GCATCCTCTT | | | GCAUCCUCUU |
| | | | | | | ACCCGCCCAG | | | ACCCGCCCAG |
| | | | | | | GAAAGAGATA | | | GAAAGAGAUA |
| | | | | | | ACTGGTTGCA | | | ACUGGUUGCA |
| | | | | | | TCATACCCAT | | | UCAUACCCAU |
| | | | | | | CCTCTCCAGG | | | CCUCUCCAGG |
| | | | | | | CGAGCCTCGT | | | CGAGCCUCGU |
| | | | | | | GGGACCGG | | | GGGACCGG |
| | | | | | | [SEQ ID | | | [SEQ ID |
| | | | | | | NO: | | | NO: |
| | | | | | | 2138] | | | 2140] |
| miR- | 3043 | −2.80802 | 0.325474 | TTTGGTGCAA | CCGGTTTGTT | TCAAGCCATG | UUUGGUGCAA | CCGGUUUGUU | UCAAGCCAUG |
| 144 | | | | AACAAACAGG | TATGCACCAA | CTTCCTGTGC | AACAAACAGG | UAUGCACCAA | CUUCCUGUGC |
| | | | | CT | A | CCCCAGTGGG | CU | A | CCCCAGUGGG |
| | | | | [SEQ ID | [SEQ ID | GCCCTGGCTC | [SEQ ID | [SEQ ID | GCCCUGGCUC |
| | | | | NO: | NO: | CGGTTTGTTT | NO: | NO: | CGGUUUGUUU |
| | | | | 1615] | 1635] | ATGCACCAAA | 1812] | 1830] | AUGCACCAAA |
| | | | | | | AGTTTGCGAT | | | AGUUUGCGAU |
| | | | | | | GAGACACTTT | | | GAGACACUUU |
| | | | | | | GGTGCAAAAC | | | GGUGCAAAAC |
| | | | | | | AAACAGGAGT | | | AAACAGGAGU |
| | | | | | | CCGGGCACCC | | | CCGGGCACCC |
| | | | | | | CCAGCTCTGG | | | CCAGCUCUGG |
| | | | | | | AGCCTGACAA | | | AGCCUGACAA |
| | | | | | | GGAGGACA | | | GGAGGACA |
| | | | | | | [SEQ ID | | | [SEQ ID |
| | | | | | | NO: | | | NO: |
| | | | | | | 1713] | | | 1910] |
| miR- | 2602 | −2.76068 | −0.25422 | TTTAGTAGTT | TCGATGGATC | TCAAGCCATG | UUUAGUAGUU | UCGAUGGAUC | UCAAGCCAUG |
| 144 | | | | GATCCATAGA | ATACTACTAA | CTTCCTGTGC | GAUCCAUAGA | AUACUACUAA | CUUCCUGUGC |
| | | | | TT | A | CCCCAGTGGG | UU | A | CCCCAGUGGG |
| | | | | [SEQ ID | [SEQ ID | GCCCTGGCTT | [SEQ ID | [SEQ ID | GCCCUGGCUU |
| | | | | NO: | NO: | CGATGGATCA | NO: | NO: | CGAUGGAUCA |
| | | | | 1616] | 1636] | TACTACTAAA | 202] | 1831] | UACUACUAAA |
| | | | | | | AGTTTGCGAT | (Same | | AGUUUGCGAU |
| | | | | | | GAGACACTTT | guide | | GAGACACUUU |
| | | | | | | AGTAGTTGAT | as | | AGUAGUUGAU |
| | | | | | | CCATAGAAGT | XD- | | CCAUAGAAGU |
| | | | | | | CCGGGCACCC | 14837) | | CCGGGCACCC |
| | | | | | | CCAGCTCTGG | | | CCAGCUCUGG |
| | | | | | | AGCCTGACAA | | | AGCCUGACAA |
| | | | | | | GGAGGACA | | | GGAGGACA |
| | | | | | | [SEQ ID | | | [SEQ ID |
| | | | | | | NO: | | | NO: |
| | | | | | | 1714] | | | 1911] |
| miR- | 3255 | −2.47679 | −0.08595 | ATTGCGTGGA | ACAAGCTTAC | TCAAGCCATG | AUUGCGUGGA | ACAAGCUUAC | UCAAGCCAUG |
| 144 | | | | GTAAGCTGGT | TGCCACGCAA | CTTCCTGTGC | GUAAGCUGGU | UGCCACGCAA | CUUCCUGUGC |
| | | | | GG | T | CCCCAGTGGG | GG | U | CCCCAGUGGG |
| | | | | [SEQ ID | [SEQ ID | GCCCTGGCTA | [SEQ ID | [SEQ ID | GCCCUGGCUA |
| | | | | NO: | NO: | CAAGCTTACT | NO: | NO: | CAAGCUUACU |
| | | | | 617] | 1660] | GCCACGCAAT | 306] | 1855] | GCCACGCAAU |
| | | | | | | AGTTTGCGAT | (Same | | AGUUUGCGAU |
| | | | | | | GAGACACATT | guide | | GAGACACAUU |
| | | | | | | GCGTGGAGTA | as | | GCGUGGAGUA |
| | | | | | | AGCTGGTAGT | XD- | | AGCUGGUAGU |
| | | | | | | CCGGGCACCC | 14889) | | CCGGGCACCC |
| | | | | | | CCAGCTCTGG | | | CCAGCUCUGG |
| | | | | | | AGCCTGACAA | | | AGCCUGACAA |
| | | | | | | GGAGGACA | | | GGAGGACA |
| | | | | | | [SEQ ID | | | [SEQ ID |
| | | | | | | NO: | | | NO: |
| | | | | | | 1744] | | | 1941] |
| miR- | 1231 | −2.16776 | −0.36035 | TTCACTTTAG | GCGATCAGTG | TCAAGCCATG | UUCACUUUAG | GCGAUCAGUG | UCAAGCCAUG |
| 144 | | | | CACTGATAGC | CATAAAGTGA | CTTCCTGTGC | CACUGAUAGC | CAUAAAGUGA | CUUCCUGUGC |
| | | | | AG | A | CCCCAGTGGG | AG | A | CCCCAGUGGG |
| | | | | [SEQ ID | [SEQ ID | GCCCTGGCTG | [SEQ ID | [SEQ ID | GCCCUGGCUG |
| | | | | NO: | NO: | CGATCAGTGC | NO: | NO: | CGAUCAGUGC |
| | | | | 1627] | 2141] | ATAAAGTGAA | 1825] | 2143] | AUAAAGUGAA |
| | | | | | | AGTTTGCGAT | | | AGUUUGCGAU |
| | | | | | | GAGACACTTC | | | GAGACACUUC |
| | | | | | | ACTTTAGCAC | | | ACUUUAGCAC |
| | | | | | | TGATAGCAGT | | | UGAUAGCAGU |
| | | | | | | CCGGGCACCC | | | CCGGGCACCC |
| | | | | | | CCAGCTCTGG | | | CCAGCUCUGG |
| | | | | | | AGCCTGACAA | | | AGCCUGACAA |
| | | | | | | GGAGGACA | | | GGAGGACA |
| | | | | | | [SEQ ID | | | [SEQ ID |
| | | | | | | NO: | | | NO: |
| | | | | | | 2142] | | | 2144] |
| miR- | 3845 | −2.11363 | −0.36405 | TTGAACGTGA | TCAATCCTTC | TCAAGCCATG | UUGAACGUGA | UCAAUCCUUC | UCAAGCCAUG |
| 144 | | | | GAAGGATGGA | TGCACGTTCA | CTTCCTGTGC | GAAGGAUGGA | UGCACGUUCA | CUUCCUGUGC |
| | | | | TC | A | CCCCAGTGGG | UC | A | CCCCAGUGGG |
| | | | | [SEQ ID | [SEQ ID | GCCCTGGCTT | [SEQ ID | [SEQ ID | GCCCUGGCUU |
| | | | | NO: | NO: | CAATCCTTCT | NO: | NO: | CAAUCCUUCU |
| | | | | 696] | 2145] | GCACGTTCAA | 1224] | 2147] | GCACGUUCAA |
| | | | | | | AGTTTGCGAT | | | AGUUUGCGAU |
| | | | | | | GAGACACTTG | | | GAGACACUUG |
| | | | | | | AACGTGAGAA | | | AACGUGAGAA |
| | | | | | | GGATGGAAGT | | | GGAUGGAAGU |
| | | | | | | CCGGGCACCC | | | CCGGGCACCC |
| | | | | | | CCAGCTCTGG | | | CCAGCUCUGG |
| | | | | | | AGCCTGACAA | | | AGCCUGACAA |
| | | | | | | GGAGGACA | | | GGAGGACA |
| | | | | | | [SEQ ID | | | [SEQ ID |
| | | | | | | NO: | | | NO: |
| | | | | | | 2146] | | | 2148] |
| miR- | 915 | −2.04132 | −0.09574 | AAATCGTAGA | ACGGCCTCAG | TCAAGCCATG | AAAUCGUAGA | ACGGCCUCAG | UCAAGCCAUG |
| 144 | | | | CTGAGGCAGT | TGCTACGATT | CTTCCTGTGC | CUGAGGCAGU | UGCUACGAUU | CUUCCUGUGC |
| | | | | CC | T | CCCCAGTGGG | CC | U | CCCCAGUGGG |
| | | | | [SEQ ID | [SEQ ID | GCCCTGGCTA | [SEQ ID | [SEQ ID | GCCCUGGCUA |
| | | | | NO: | NO: | CGGCCTCAGT | NO: | NO: | CGGCCUCAGU |
| | | | | 2149] | 2150] | GCTACGATTT | 2152] | 2153] | GCUACGAUUU |
| | | | | | | AGTTTGCGAT | | | AGUUUGCGAU |
| | | | | | | GAGACACAAA | | | GAGACACAAA |
| | | | | | | TCGTAGACTG | | | UCGUAGACUG |
| | | | | | | AGGCAGTAGT | | | AGGCAGUAGU |
| | | | | | | CCGGGCACCC | | | CCGGGCACCC |
| | | | | | | CCAGCTCTGG | | | CCAGCUCUGG |
| | | | | | | AGCCTGACAA | | | AGCCUGACAA |
| | | | | | | GGAGGACA | | | GGAGGACA |
| | | | | | | [SEQ ID | | | [SEQ ID |
| | | | | | | NO: | | | NO: |
| | | | | | | 2151] | | | 2154] |
| miR- | 3301 | −1.88293 | 0.071952 | TGAACAAGGG | CCCAATCAGC | TCAAGCCATG | UGAACAAGGG | CCCAAUCAGC | UCAAGCCAUG |
| 144 | | | | GCTGATTTGG | CGCCTTGTTC | CTTCCTGTGC | GCUGAUUUGG | CGCCUUGUUC | CUUCCUGUGC |
| | | | | GA | A | CCCCAGTGGG | GA | A | CCCCAGUGGG |
| | | | | [SEQ ID | [SEQ ID | GCCCTGGCTC | [SEQ ID | [SEQ ID | GCCCUGGCUC |
| | | | | NO: | NO: | CCAATCAGCC | NO: | NO: | CCAAUCAGCC |
| | | | | 687] | 2155] | GCCTTGTTCA | 1215] | 2157] | GCCUUGUUCA |
| | | | | | | AGTTTGCGAT | | | AGUUUGCGAU |
| | | | | | | GAGACACTGA | | | GAGACACUGA |
| | | | | | | ACAAGGGGCT | | | ACAAGGGGCU |
| | | | | | | GATTTGGAGT | | | GAUUUGGAGU |
| | | | | | | CCGGGCACCC | | | CCGGGCACCC |
| | | | | | | CCAGCTCTGG | | | CCAGCUCUGG |
| | | | | | | AGCCTGACAA | | | AGCCUGACAA |
| | | | | | | GGAGGACA | | | GGAGGACA |
| | | | | | | [SEQ ID | | | [SEQ ID |
| | | | | | | NO: | | | NO: |
| | | | | | | 2156] | | | 2158] |
| miR- | 2932 | −1.63 | 0.064183 | TTTGGCTGAG | CGGTCCTTCT | TCAAGCCATG | UUUGGCUGAG | CGGUCCUUCU | UCAAGCCAUG |
| 144 | | | | AGAAGGAACG | CATCAGCCAA | CTTCCTGTGC | AGAAGGAACG | CAUCAGCCAA | CUUCCUGUGC |
| | | | | TG | A | CCCCAGTGGG | UG | A | CCCCAGUGGG |
| | | | | [SEQ ID | [SEQ ID | GCCCTGGCTC | [SEQ ID | [SEQ ID | GCCCUGGCUC |
| | | | | NO: | NO: | GGTCCTTCTC | NO: | NO: | GGUCCUUCUC |
| | | | | 672] | 2159] | ATCAGCCAAA | 1200] | 2161] | AUCAGCCAAA |
| | | | | | | AGTTTGCGAT | | | AGUUUGCGAU |
| | | | | | | GAGACACTTT | | | GAGACACUUU |
| | | | | | | GGCTGAGAGA | | | GGCUGAGAGA |
| | | | | | | AGGAACGAGT | | | AGGAACGAGU |
| | | | | | | CCGGGCACCC | | | CCGGGCACCC |
| | | | | | | CCAGCTCTGG | | | CCAGCUCUGG |
| | | | | | | AGCCTGACAA | | | AGCCUGACAA |
| | | | | | | GGAGGACA | | | GGAGGACA |
| | | | | | | [SEQ ID | | | [SEQ ID |
| | | | | | | NO: | | | NO: |
| | | | | | | 2160] | | | 2162] |
| miR- | 3842 | −1.57752 | −0.42898 | AACGTGAGAA | CGCTCCATCC | TCAAGCCATG | AACGUGAGAA | CGCUCCAUCC | UCAAGCCAUG |
| 144 | | | | GGATGGATCG | TATCTCACGT | CTTCCTGTGC | GGAUGGAUCG | UAUCUCACGU | CUUCCUGUGC |
| | | | | TA | T | CCCCAGTGGG | UA | U | CCCCAGUGGG |
| | | | | [SEQ ID | [SEQ ID | GCCCTGGCTC | [SEQ ID | [SEQ ID | GCCCUGGCUC |
| | | | | NO: | NO: | GCTCCATCCT | NO: | NO: | GCUCCAUCCU |
| | | | | 1625] | 2163] | ATCTCACGTT | 1824] | 2165] | AUCUCACGUU |
| | | | | | | AGTTTGCGAT | | | AGUUUGCGAU |
| | | | | | | GAGACACAAC | | | GAGACACAAC |
| | | | | | | GTGAGAAGGA | | | GUGAGAAGGA |
| | | | | | | TGGATCGAGT | | | UGGAUCGAGU |
| | | | | | | CCGGGCACCC | | | CCGGGCACCC |
| | | | | | | CCAGCTCTGG | | | CCAGCUCUGG |
| | | | | | | AGCCTGACAA | | | AGCCUGACAA |
| | | | | | | GGAGGACA | | | GGAGGACA |
| | | | | | | [SEQ ID | | | [SEQ ID |
| | | | | | | NO: | | | NO: |
| | | | | | | 2164] | | | 2166] |
| miR- | 2353 | −1.4808 | 0.451244 | TTCGATGCAG | CCGGCTAGTC | TCAAGCCATG | UUCGAUGCAG | CCGGCUAGUC | UCAAGCCAUG |
| 144 | | | | GACTAGCAGG | CATGCATCGA | CTTCCTGTGC | GACUAGCAGG | CAUGCAUCGA | CUUCCUGUGC |
| | | | | CG | A | CCCCAGTGGG | CG | A | CCCCAGUGGG |
| | | | | [SEQ ID | [SEQ ID | GCCCTGGCTC | [SEQ ID | [SEQ ID | GCCCUGGCUC |
| | | | | NO: | NO: | CGGCTAGTCC | NO: | NO: | CGGCUAGUCC |
| | | | | 612] | 2167] | ATGCATCGAA | 166] | 2169] | AUGCAUCGAA |
| | | | | | | AGTTTGCGAT | (Same | | AGUUUGCGAU |
| | | | | | | GAGACACTTC | as | | GAGACACUUC |
| | | | | | | GATGCAGGAC | XD- | | GAUGCAGGAC |
| | | | | | | TAGCAGGAGT | 14819) | | UAGCAGGAGU |
| | | | | | | CCGGGCACCC | | | CCGGGCACCC |
| | | | | | | CCAGCTCTGG | | | CCAGCUCUGG |
| | | | | | | AGCCTGACAA | | | AGCCUGACAA |
| | | | | | | GGAGGACA | | | GGAGGACA |
| | | | | | | [SEQ ID | | | [SEQ ID |
| | | | | | | NO: | | | NO: |
| | | | | | | 2168] | | | 2170] |
| miR- | 3302 | −2.52291 | 0.018122 | TTGAACAAGG | CCCAAATCGC | CTGGAGGCTT | UUGAACAAGG | CCCAAAUCGC | CUGGAGGCUU |
| 155E | | | | GGCTGATTTG | CCTTGTTCAA | GCTTTGGGCT | GGCUGAUUUG | CCUUGUUCAA | GCUUUGGGCU |
| | | | | GG | [SEQ ID | GTATGCTGTT | GG | [SEQ ID | GUAUGCUGUU |
| | | | | [SEQ ID | NO: | GAACAAGGGG | [SEQ ID | NO: | GAACAAGGGG |
| | | | | NO: | 1652] | CTGATTTGGG | NO: | 1847] | CUGAUUUGGG |
| | | | | 688] | | TTTTGGCCTC | 1216] | | UUUUGGCCUC |
| | | | | | | TGACTGACCC | | | UGACUGACCC |
| | | | | | | AAATCGCCCT | | | AAAUCGCCCU |
| | | | | | | TGTTCAACAG | | | UGUUCAACAG |
| | | | | | | GACAAGGCCC | | | GACAAGGCCC |
| | | | | | | TTTATCAGCA | | | UUUAUCAGCA |
| | | | | | | CTCACATGGA | | | CUCACAUGGA |
| | | | | | | ACAAATGGCC | | | ACAAAUGGCC |
| | | | | | | ACCGTGGG | | | ACCGUGGG |
| | | | | | | [SEQ ID | | | [SEQ ID |
| | | | | | | NO: | | | NO: |
| | | | | | | 1734] | | | 1931] |
| miR- | 2586 | −2.5179 | −0.10172 | TAGATTCAGA | CCAAGTTCAC | CTGGAGGCTT | UAGAUUCAGA | CCAAGUUCAC | CUGGAGGCUU |
| 155E | | | | AGTAGAACTT | TCTGAATCTA | GCTTTGGGCT | AGUAGAACUU | UCUGAAUCUA | GCUUUGGGCU |
| | | | | GG | [SEQ ID | GTATGCTGTA | GG | [SEQ ID | GUAUGCUGUA |
| | | | | [SEQ ID | NO: | GATTCAGAAG | [SEQ ID | NO: | GAUUCAGAAG |
| | | | | NO: | 1653] | TAGAACTTGG | NO: | 1848] | UAGAACUUGG |
| | | | | 1621] | | TTTTGGCCTC | 1816] | | UUUUGGCCUC |
| | | | | | | TGACTGACCA | | | UGACUGACCA |
| | | | | | | AGTTCACTCT | | | AGUUCACUCU |
| | | | | | | GAATCTACAG | | | GAAUCUACAG |
| | | | | | | GACAAGGCCC | | | GACAAGGCCC |
| | | | | | | TTTATCAGCA | | | UUUAUCAGCA |
| | | | | | | CTCACATGGA | | | CUCACAUGGA |
| | | | | | | ACAAATGGCC | | | ACAAAUGGCC |
| | | | | | | ACCGTGGG | | | ACCGUGGG |
| | | | | | | [SEQ ID | | | [SEQ ID |
| | | | | | | NO: | | | NO: |
| | | | | | | 1736] | | | 1933] |
| miR- | 1755 | −2.39604 | −0.03915 | TCGGGTTGAA | CACACTTCGA | CTGGAGGCTT | UCGGGUUGAA | CACACUUCGA | CUGGAGGCUU |
| 155E | | | | ATCTGAAGTG | TTCAACCCGA | GCTTTGGGCT | AUCUGAAGUG | UUCAACCCGA | GCUUUGGGCU |
| | | | | TG | [SEQ ID | GTATGCTGTC | UG | [SEQ ID | GUAUGCUGUC |
| | | | | [SEQ ID | NO: | GGGTTGAAAT | [SEQ ID | NO: | GGGUUGAAAU |
| | | | | NO: | 1676] | CTGAAGTGTG | NO: | 1871] | CUGAAGUGUG |
| | | | | 657] | | TTTTGGCCTC | 1185] | | UUUUGGCCUC |
| | | | | | | TGACTGACAC | | | UGACUGACAC |
| | | | | | | ACTTCGATTC | | | ACUUCGAUUC |
| | | | | | | AACCCGACAG | | | AACCCGACAG |
| | | | | | | GACAAGGCCC | | | GACAAGGCCC |
| | | | | | | TTTATCAGCA | | | UUUAUCAGCA |
| | | | | | | CTCACATGGA | | | CUCACAUGGA |
| | | | | | | ACAAATGGCC | | | ACAAAUGGCC |
| | | | | | | ACCGTGGG | | | ACCGUGGG |
| | | | | | | [SEQ ID | | | [SEQ ID |
| | | | | | | NO: | | | NO: |
| | | | | | | 1766] | | | 1963] |
| miR- | 3273 | −2.36984 | −0.0801 | TAGGACTGTA | CAATATGTGC | CTGGAGGCTT | UAGGACUGUA | CAAUAUGUGC | CUGGAGGCUU |
| 155E | | | | GGCAACATAT | CACAGTCCTA | GCTTTGGGCT | GGCAACAUAU | CACAGUCCUA | GCUUUGGGCU |
| | | | | TG | [SEQ ID | GTATGCTGTA | UG | [SEQ ID | GUAUGCUGUA |
| | | | | [SEQ ID | NO: | GGACTGTAGG | [SEQ ID | NO: | GGACUGUAGG |
| | | | | NO: | 1683] | CAACATATTG | NO: | 1878] | CAACAUAUUG |
| | | | | 1628] | | TTTTGGCCTC | 1821] | | UUUUGGCCUC |
| | | | | | | TGACTGACAA | | | UGACUGACAA |
| | | | | | | TATGTGCCAC | | | UAUGUGCCAC |
| | | | | | | AGTCCTACAG | | | AGUCCUACAG |
| | | | | | | GACAAGGCCC | | | GACAAGGCCC |
| | | | | | | TTTATCAGCA | | | UUUAUCAGCA |
| | | | | | | CTCACATGGA | | | CUCACAUGGA |
| | | | | | | ACAAATGGCC | | | ACAAAUGGCC |
| | | | | | | ACCGTGGG | | | ACCGUGGG |
| | | | | | | [SEQ ID | | | [SEQ ID |
| | | | | | | NO: | | | NO: |
| | | | | | | 1774] | | | 1971] |
| miR- | 3272 | −2.34055 | −0.19485 | AGGACTGTAG | GCAATATGTG | CTGGAGGCTT | AGGACUGUAG | GCAAUAUGUG | CUGGAGGCUU |
| 155E | | | | GCAACATATT | CTACAGTCCT | GCTTTGGGCT | GCAACAUAUU | CUACAGUCCU | GCUUUGGGCU |
| | | | | GC | [SEQ ID | GTATGCTGAG | GC | [SEQ ID | GUAUGCUGAG |
| | | | | [SEQ ID | NO: | GACTGTAGGC | [SEQ ID | NO: | GACUGUAGGC |
| | | | | NO: | 1687] | AACATATTGC | NO: | 1882] | AACAUAUUGC |
| | | | | 1618] | | TTTTGGCCTC | 1814] | | UUUUGGCCUC |
| | | | | | | TGACTGAGCA | | | UGACUGAGCA |
| | | | | | | ATATGTGCTA | | | AUAUGUGCUA |
| | | | | | | CAGTCCTCAG | | | CAGUCCUCAG |
| | | | | | | GACAAGGCCC | | | GACAAGGCCC |
| | | | | | | TTTATCAGCA | | | UUUAUCAGCA |
| | | | | | | CTCACATGGA | | | CUCACAUGGA |
| | | | | | | ACAAATGGCC | | | ACAAAUGGCC |
| | | | | | | ACCGTGGG | | | ACCGUGGG |
| | | | | | | [SEQ ID | | | [SEQ ID |
| | | | | | | NO: | | | NO: |
| | | | | | | 1780] | | | 1977] |
| miR- | 3330 | −2.26554 | 0.033188 | TATGCTGAGA | CCACATTACA | CTGGAGGCTT | UAUGCUGAGA | CCACAUUACA | CUGGAGGCUU |
| 155E | | | | CTGATAATGT | GCTCAGCATA | GCTTTGGGCT | CUGAUAAUGU | GCUCAGCAUA | GCUUUGGGCU |
| | | | | GG | [SEQ ID | GTATGCTGTA | GG | [SEQ ID | GUAUGCUGUA |
| | | | | [SEQ ID | NO: | TGCTGAGACT | [SEQ ID | NO: | UGCUGAGACU |
| | | | | NO: | 1700] | GATAATGTGG | NO: | 1895] | GAUAAUGUGG |
| | | | | 1614] | | TTTTGGCCTC | 1811] | | UUUUGGCCUC |
| | | | | | | TGACTGACCA | | | UGACUGACCA |
| | | | | | | CATTACAGCT | | | CAUUACAGCU |
| | | | | | | CAGCATACAG | | | CAGCAUACAG |
| | | | | | | GACAAGGCCC | | | GACAAGGCCC |
| | | | | | | TTTATCAGCA | | | UUUAUCAGCA |
| | | | | | | CTCACATGGA | | | CUCACAUGGA |
| | | | | | | ACAAATGGCC | | | ACAAAUGGCC |
| | | | | | | ACCGTGGG | | | ACCGUGGG |
| | | | | | | [SEQ ID | | | [SEQ ID |
| | | | | | | NO: | | | NO: |
| | | | | | | 1793] | | | 1990] |
| miR- | 1162 | −2.26036 | −0.20831 | AACTGTACCA | CAGACTTTTT | CTGGAGGCTT | AACUGUACCA | CAGACUUUUU | CUGGAGGCUU |
| 155E | | | | CAACAAAGTC | GGGTACAGTT | GCTTTGGGCT | CAACAAAGUC | GGGUACAGUU | GCUUUGGGCU |
| | | | | TG | [SEQ ID | GTATGCTGAA | UG | [SEQ ID | GUAUGCUGAA |
| | | | | [SEQ ID | NO: | CTGTACCACA | [SEQ ID | NO: | CUGUACCACA |
| | | | | NO: | 1706] | ACAAAGTCTG | NO: | 1905] | ACAAAGUCUG |
| | | | | 652] | | TTTTGGCCTC | 1180] | | UUUUGGCCUC |
| | | | | | | TGACTGACAG | | | UGACUGACAG |
| | | | | | | ACTTTTTGGG | | | ACUUUUUGGG |
| | | | | | | TACAGTTCAG | | | UACAGUUCAG |
| | | | | | | GACAAGGCCC | | | GACAAGGCCC |
| | | | | | | TTTATCAGCA | | | UUUAUCAGCA |
| | | | | | | CTCACATGGA | | | CUCACAUGGA |
| | | | | | | ACAAATGGCC | | | ACAAAUGGCC |
| | | | | | | ACCGTGGG | | | ACCGUGGG |
| | | | | | | [SEQ ID | | | [SEQ ID |
| | | | | | | NO: | | | NO: |
| | | | | | | 1801] | | | 1998] |
| miR- | 1580 | −2.18059 | −0.26086 | ACTGGAATTT | AGCAGTTCGA | CTGGAGGCTT | ACUGGAAUUU | AGCAGUUCGA | CUGGAGGCUU |
| 155E | | | | CTCTGAACTG | GAATTCCAGT | GCTTTGGGCT | CUCUGAACUG | GAAUUCCAGU | GCUUUGGGCU |
| | | | | CT | [SEQ ID | GTATGCTGAC | CU | [SEQ ID | GUAUGCUGAC |
| | | | | [SEQ ID | NO: | TGGAATTTCT | [SEQ ID | NO: | UGGAAUUUCU |
| | | | | NO: | 2171] | CTGAACTGCT | NO: | 2173] | CUGAACUGCU |
| | | | | 1622] | | TTTTGGCCTC | 1817] | | UUUUGGCCUC |
| | | | | | | TGACTGAAGC | | | UGACUGAAGC |
| | | | | | | AGTTCGAGAA | | | AGUUCGAGAA |
| | | | | | | TTCCAGTCAG | | | UUCCAGUCAG |
| | | | | | | GACAAGGCCC | | | GACAAGGCCC |
| | | | | | | TTTATCAGCA | | | UUUAUCAGCA |
| | | | | | | CTCACATGGA | | | CUCACAUGGA |
| | | | | | | ACAAATGGCC | | | ACAAAUGGCC |
| | | | | | | ACCGTGGG | | | ACCGUGGG |
| | | | | | | [SEQ ID | | | [SEQ ID |
| | | | | | | NO: | | | NO: |
| | | | | | | 2172] | | | 2174] |
| miR- | 1436 | −2.08545 | 0.133624 | TGAGTTATCT | GCCCTTAGAA | CTGGAGGCTT | UGAGUUAUCU | GCCCUUAGAA | CUGGAGGCUU |
| 155E | | | | CTTTCTAAGG | GGATAACTCA | GCTTTGGGCT | CUUUCUAAGG | GGAUAACUCA | GCUUUGGGCU |
| | | | | GC | [SEQ ID | GTATGCTGTG | GC | [SEQ ID | GUAUGCUGUG |
| | | | | [SEQ ID | NO: | AGTTATCTCT | [SEQ ID | NO: | AGUUAUCUCU |
| | | | | NO: | 1710] | TTCTAAGGGC | NO: | 1900] | UUCUAAGGGC |
| | | | | 1632] | | TTTTGGCCTC | 1827] | | UUUUGGCCUC |
| | | | | | | TGACTGAGCC | | | UGACUGAGCC |
| | | | | | | CTTAGAAGGA | | | CUUAGAAGGA |
| | | | | | | TAACTCACAG | | | UAACUCACAG |
| | | | | | | GACAAGGCCC | | | GACAAGGCCC |
| | | | | | | TTTATCAGCA | | | UUUAUCAGCA |
| | | | | | | CTCACATGGA | | | CUCACAUGGA |
| | | | | | | ACAAATGGCC | | | ACAAAUGGCC |
| | | | | | | ACCGTGGG | | | ACCGUGGG |
| | | | | | | [SEQ ID | | | [SEQ ID |
| | | | | | | NO: | | | NO: |
| | | | | | | 2175] | | | 2176] |
| miR- | 3270 | −2.03222 | 0.002948 | TACTGTAGGC | ACGCAATAGT | CTGGAGGCTT | UACUGUAGGC | ACGCAAUAGU | CUGGAGGCUU |
| 155E | | | | AACATATTGC | TCCTACAGTA | GCTTTGGGCT | AACAUAUUGC | UCCUACAGUA | GCUUUGGGCU |
| | | | | GT | | GTATGCTGTA | GU | [SEQ ID | GUAUGCUGUA |
| | | | | [SEQ ID | [SEQ ID | CTGTAGGCAA | [SEQ ID | NO: | CUGUAGGCAA |
| | | | | NO: | NO: | CATATTGCGT | NO: | 2179] | CAUAUUGCGU |
| | | | | 2080] | 2177] | TTTTGGCCTC | 2083] | | UUUUGGCCUC |
| | | | | | | TGACTGAACG | | | UGACUGAACG |
| | | | | | | CAATAGTTCC | | | CAAUAGUUCC |
| | | | | | | TACAGTACAG | | | UACAGUACAG |
| | | | | | | GACAAGGCCC | | | GACAAGGCCC |
| | | | | | | TTTATCAGCA | | | UUUAUCAGCA |
| | | | | | | CTCACATGGA | | | CUCACAUGGA |
| | | | | | | ACAAATGGCC | | | ACAAAUGGCC |
| | | | | | | ACCGTGGG | | | ACCGUGGG |
| | | | | | | [SEQ ID | | | [SEQ ID |
| | | | | | | NO: | | | NO: |
| | | | | | | 2178] | | | 2180] |
| miR- | 3330 | −2.86183 | 0.46905 | TATGCTGAGA | CCATTATCAG | GAGCTCAGTC | UAUGCUGAGA | CCAUUAUCAG | GAGCUCAGUC |
| 190a | | | | CTGATAATGT | TCTCAGCACC | AAACCTGGAT | CUGAUAAUGU | UCUCAGCACC | AAACCUGGAU |
| | | | | GG | [SEQ ID | GCCTTTTCTG | GG | [SEQ ID | GCCUUUUCUG |
| | | | | [SEQ ID | NO: | CAGGCCTCTG | [SEQ ID | NO: | CAGGCCUCUG |
| | | | | NO: | 1634] | TGTATGCTGA | NO: | 1829] | UGUAUGCUGA |
| | | | | 1614] | | G | 1811] | | G |
| | | | | | | ACTGATAATG | | | ACUGAUAAUG |
| | | | | | | TGGTGTTATT | | | UGGUGUUAUU |
| | | | | | | TAATCCACCA | | | UAAUCCACCA |
| | | | | | | TTATCAGTCT | | | UUAUCAGUCU |
| | | | | | | CAGCACCCTA | | | CAGCACCCUA |
| | | | | | | CAGTGTCTTG | | | CAGUGUCUUG |
| | | | | | | CCCTGTCTCC | | | CCCUGUCUCC |
| | | | | | | GGGGGTTCCT | | | GGGGGUUCCU |
| | | | | | | AATAAAG | | | AAUAAAG |
| | | | | | | [SEQ ID | | | [SEQ ID |
| | | | | | | NO: | | | NO: |
| | | | | | | 1712] | | | 1909] |
| miR- | 3302 | −2.64186 | 0.124524 | TTGAACAAGG | CCCATCAGCC | GAGCTCAGTC | UUGAACAAGG | CCCAUCAGCC | GAGCUCAGUC |
| 190a | | | | GGCTGATTTG | CCTTGTTCCC | AAACCTGGAT | GGCUGAUUUG | CCUUGUUCCC | AAACCUGGAU |
| | | | | GG | [SEQ ID | GCCTTTTCTG | GG | [SEQ ID | GCCUUUUCUG |
| | | | | [SEQ ID | NO: | CAGGCCTCTG | [SEQ ID | NO: | CAGGCCUCUG |
| | | | | NO: | 1643] | TGTTGAACAA | NO: | 1838] | UGUUGAACAA |
| | | | | 688] | | GGGGCTGATT | 1216] | | GGGGCUGAUU |
| | | | | | | TGGGTGTTAT | | | UGGGUGUUAU |
| | | | | | | TTAATCCACC | | | UUAAUCCACC |
| | | | | | | CATCAGCCCC | | | CAUCAGCCCC |
| | | | | | | TTGTTCCCCT | | | UUGUUCCCCU |
| | | | | | | ACAGTGTCTT | | | ACAGUGUCUU |
| | | | | | | GCCCTGTCTC | | | GCCCUGUCUC |
| | | | | | | CGGGGGTTCC | | | CGGGGGUUCC |
| | | | | | | TAATAAAG | | | UAAUAAAG |
| | | | | | | [SEQ ID | | | [SEQ ID |
| | | | | | | NO: | | | NO: |
| | | | | | | 1721] | | | 1918] |
| miR- | 1580 | −2.50168 | −0.06321 | ACTGGAATTT | AGCTTCAGAG | GAGCTCAGTC | ACUGGAAUUU | AGCUUCAGAG | GAGCUCAGUC |
| 190a | | | | CTCTGAACTG | AAATTCCAAG | AAACCTGGAT | CUCUGAACUG | AAAUUCCAAG | AAACCUGGAU |
| | | | | CT | [SEQ ID | GCCTTTTCTG | CU | [SEQ ID | GCCUUUUCUG |
| | | | | [SEQ ID | NO: | CAGGCCTCTG | [SEQ ID | NO: | CAGGCCUCUG |
| | | | | NO: | 1654] | TGACTGGAAT | NO: | 1849] | UGACUGGAAU |
| | | | | 1622] | | TTCTCTGAAC | 1817] | | UUCUCUGAAC |
| | | | | | | TGCTTGTTAT | | | UGCUUGUUAU |
| | | | | | | TTAATCCAAG | | | UUAAUCCAAG |
| | | | | | | CTTCAGAGAA | | | CUUCAGAGAA |
| | | | | | | ATTCCAAGCT | | | AUUCCAAGCU |
| | | | | | | ACAGTGTCTT | | | ACAGUGUCUU |
| | | | | | | GCCCTGTCTC | | | GCCCUGUCUC |
| | | | | | | CGGGGGTTCC | | | CGGGGGUUCC |
| | | | | | | TAATAAAG | | | UAAUAAAG |
| | | | | | | [SEQ ID | | | [SEQ ID |
| | | | | | | NO: | | | NO: |
| | | | | | | 1738] | | | 1911] |
| miR- | 1755 | −2.38113 | 0.329455 | TCGGGTTGAA | CACTTCAGAT | GAGCTCAGTC | UCGGGUUGAA | CACUUCAGAU | GAGCUCAGUC |
| 190a | | | | ATCTGAAGTG | TTCAACCCAC | AAACCTGGAT | AUCUGAAGUG | UUCAACCCAC | AAACCUGGAU |
| | | | | TG | [SEQ ID | GCCTTTTCTG | UG | [SEQ ID | GCCUUUUCUG |
| | | | | [SEQ ID | NO: | CAGGCCTCTG | [SEQ ID | NO: | CAGGCCUCUG |
| | | | | NO: | 1680] | TGTCGGGTTG | NO: | 1875] | UGUCGGGUUG |
| | | | | 657] | | AAATCTGAAG | 1185] | | AAAUCUGAAG |
| | | | | | | TGTGTGTTAT | | | UGUGUGUUAU |
| | | | | | | TTAATCCACA | | | UUAAUCCACA |
| | | | | | | CTTCAGATT | | | CUUCAGAU |
| | | | | | | TCAACCCACC | | | UUCAACCCAC |
| | | | | | | TACAGTGTCT | | | CUACAGUGUC |
| | | | | | | TGCCCTGTCT | | | UUGCCCUGUC |
| | | | | | | CCGGGGGTTC | | | UCCGGGGGUU |
| | | | | | | CTAATAAAG | | | CCUAAUAAAG |
| | | | | | | [SEQ ID | | | [SEQ ID |
| | | | | | | NO: | | | NO: |
| | | | | | | 1771] | | | 1968] |
| miR- | 3301 | −2.36785 | 0.195332 | TGAACAAGGG | TCCAATCAGC | GAGCTCAGTC | UGAACAAGGG | UCCAAUCAGC | GAGCUCAGUC |
| 190a | | | | GCTGATTTGG | CCCTTGTTAC | AAACCTGGAT | GCUGAUUUGG | CCCUUGUUAC | AAACCUGGAU |
| | | | | GA | [SEQ ID | GCCTTTTCTG | GA | [SEQ ID | GCCUUUUCUG |
| | | | | [SEQ ID | NO: | CAGGCCTCTG | [SEQ ID | NO: | CAGGCCUCUG |
| | | | | NO: | 1684] | TGTGAACAAG | NO: | 1879] | UGUGAACAAG |
| | | | | 687] | | GGGCTGATTT | 1215] | | GGGCUGAUUU |
| | | | | | | GGGATGTTAT | | | GGGAUGUUAU |
| | | | | | | TTAATCCATC | | | UUAAUCCAUC |
| | | | | | | CAATCAGCCC | | | CAAUCAGCCC |
| | | | | | | CTTGTTACCT | | | CUUGUUACCU |
| | | | | | | ACAGTGTCTT | | | ACAGUGUCUU |
| | | | | | | GCCCTGTCTC | | | GCCCUGUCUC |
| | | | | | | CGGGGGTTCC | | | CGGGGGUUCC |
| | | | | | | TAATAAAG | | | UAAUAAAG |
| | | | | | | [SEQ ID | | | [SEQ ID |
| | | | | | | NO: | | | NO: |
| | | | | | | 1775] | | | 1972] |
| miR- | 3043 | −2.26041 | 0.277711 | TTTGGTGCAA | AGCGTTTGTT | GAGCTCAGTC | UUUGGUGCAA | AGCGUUUGUU | GAGCUCAGUC |
| 190a | | | | AACAAACAGG | TTGCACCACC | AAACCTGGAT | AACAAACAGG | UUGCACCACC | AAACCUGGAU |
| | | | | CT | [SEQ ID | GCCTTTTCTG | CU | [SEQ ID | GCCUUUUCUG |
| | | | | [SEQ ID | NO: | CAGGCCTCTG | [SEQ ID | NO: | CAGGCCUCUG |
| | | | | NO: | 1664] | TGTTTGGTGC | NO: | 1859] | UGUUUGGUGC |
| | | | | 1615] | | AAAACAAACA | 1812] | | AAAACAAACA |
| | | | | | | GGCTTGTTAT | | | GGCUUGUUAU |
| | | | | | | TTAATCCAAG | | | UUAAUCCAAG |
| | | | | | | CGTTTGTTTT | | | CGUUUGUUUU |
| | | | | | | GCACCACCCT | | | GCACCACCCU |
| | | | | | | ACAGTGTCTT | | | ACAGUGUCUU |
| | | | | | | GCCCTGTCTC | | | GCCCUGUCUC |
| | | | | | | CGGGGGTTCC | | | CGGGGGUUCC |
| | | | | | | TAATAAAG | | | UAAUAAAG |
| | | | | | | [SEQ ID | | | [SEQ ID |
| | | | | | | NO: | | | NO: |
| | | | | | | 1800] | | | 1997] |
| miR- | 2586 | −2.24846 | 0.172174 | TAGATTCAGA | CCATTCTACT | GAGCTCAGTC | UAGAUUCAGA | CCAUUCUACU | GAGCUCAGUC |
| 190a | | | | AGTAGAACTT | TCTGAATCCC | AAACCTGGAT | AGUAGAACUU | UCUGAAUCCC | AAACCUGGAU |
| | | | | GG | [SEQ ID | GCCTTTTCTG | GG | [SEQ ID | GCCUUUUCUG |
| | | | | [SEQ ID | NO: | CAGGCCTCTG | [SEQ ID | NO: | CAGGCCUCUG |
| | | | | NO: | 1661] | TGTAGATTCA | NO: | 1856] | UGUAGAUUCA |
| | | | | 1621] | | GAAGTAGAAC | 1816] | | GAAGUAGAAC |
| | | | | | | TTGGTGTTAT | | | UUGGUGUUAU |
| | | | | | | TTAATCCACC | | | UUAAUCCACC |
| | | | | | | ATTCTACTTC | | | AUUCUACUUC |
| | | | | | | TGAATCCCCT | | | UGAAUCCCCU |
| | | | | | | ACAGTGTCTT | | | ACAGUGUCUU |
| | | | | | | GCCCT | | | GCCC |
| | | | | | | GTCTCCGGGG | | | UGUCUCCGGG |
| | | | | | | GTTCCTAATA | | | GGUUCCUAAU |
| | | | | | | AAG | | | AAAG |
| | | | | | | [SEQ ID | | | [SEQ ID |
| | | | | | | NO: | | | NO: |
| | | | | | | 1803] | | | 2000] |
| miR- | 967 | −2.23671 | −0.08895 | ACTGATGTAA | TGGCATATAC | GAGCTCAGTC | ACUGAUGUAA | UGGCAUAUAC | GAGCUCAGUC |
| 190a | | | | GTATATGAAC | TTACATCAAG | AAACCTGGAT | GUAUAUGAAC | UUACAUCAAG | AAACCUGGAU |
| | | | | CA | [SEQ ID | GCCTTTTCTG | CA | [SEQ ID | GCCUUUUCUG |
| | | | | [SEQ ID | NO: | CAGGCCTCTG | [SEQ ID | NO: | CAGGCCUCUG |
| | | | | NO: | 1642] | TGACTGATGT | NO: | 1837] | UGACUGAUGU |
| | | | | 1619] | | AAGTATATGA | 1815] | | AAGUAUAUGA |
| | | | | | | ACCATGTTAT | | | ACCAUGUUAU |
| | | | | | | TTAATCCATG | | | UUAAUCCAUG |
| | | | | | | GCATATACTT | | | GCAUAUACUU |
| | | | | | | ACATCAAGCT | | | ACAUCAAGCU |
| | | | | | | ACAGTGTCTT | | | ACAGUGUCUU |
| | | | | | | GCCCTGTCTC | | | GCCCUGUCUC |
| | | | | | | CGGGGGTTCC | | | CGGGGGUUCC |
| | | | | | | TAATAAAG | | | UAAUAAAG |
| | | | | | | [SEQ ID | | | [SEQ ID |
| | | | | | | NO: | | | NO: |
| | | | | | | 1809] | | | 2006] |
| miR- | 3255 | −2.21465 | 0.46382 | ATTGCGTGGA | CCAAGCTTAC | GAGCTCAGTC | AUUGCGUGGA | CCAAGCUUAC | GAGCUCAGUC |
| 190a | | | | GTAAGCTGGT | TCCACGCACG | AAACCTGGAT | GUAAGCUGGU | UCCACGCACG | AAACCUGGAU |
| | | | | GG | [SEQ ID | GCCTTTTCTG | GG | [SEQ ID | GCCUUUUCUG |
| | | | | [SEQ ID | NO: | CAGGCCTCTG | [SEQ ID | NO: | CAGGCCUCUG |
| | | | | NO: | 1697] | TGATTGCGTG | NO: | 1892] | UGAUUGCGUG |
| | | | | 617] | | GAGTAAGCTG | 306] | | GAGUAAGCUG |
| | | | | | | GTGGTGTTAT | (Same | | GUGGUGUUAU |
| | | | | | | TTAATCCACC | guide | | UUAAUCCACC |
| | | | | | | AAGCTTACTC | as | | AAGCUUACUC |
| | | | | | | CACGCACGCT | XD- | | CACGCACGCU |
| | | | | | | ACAGTGTCTT | 14889) | | ACAGUGUCUU |
| | | | | | | GCCCTGTCTC | | | GCCCUGUCUC |
| | | | | | | CGGGGGTTCC | | | CGGGGGUUCC |
| | | | | | | TAATAAAG | | | UAAUAAAG |
| | | | | | | [SEQ ID | | | [SEQ ID |
| | | | | | | NO: | | | NO: |
| | | | | | | 2181] | | | 2182] |
| miR- | 3341 | −2.17489 | 0.322497 | ATAGACATGA | GTCAGCATCC | GAGCTCAGTC | AUAGACAUGA | GUCAGCAUCC | GAGCUCAGUC |
| 190a | | | | GGATGCTGAG | TCATGTCTCG | AAACCTGGAT | GGAUGCUGAG | UCAUGUCUCG | AAACCUGGAU |
| | | | | AC | [SEQ ID | GCCTTTTCTG | AC | [SEQ ID | GCCUUUUCUG |
| | | | | [SEQ ID | NO: | CAGGCCTCTG | [SEQ ID | NO: | CAGGCCUCUG |
| | | | | NO: | 1703] | TGATAGACAT | NO: | 1898] | UGAUAGACAU |
| | | | | 1617] | | GAGGATGCTG | 1813] | | GAGGAUGCUG |
| | | | | | | AGACTGTTAT | | | AGACUGUUAU |
| | | | | | | TTAATCCAGT | | | UUAAUCCAGU |
| | | | | | | CAGCATCCTC | | | CAGCAUCCUC |
| | | | | | | ATGTCTCGCT | | | AUGUCUCGCU |
| | | | | | | ACAGTGTCTT | | | ACAGUGUCUU |
| | | | | | | GCCCTGTCTC | | | GCCCUGUCUC |
| | | | | | | CGGGGGTTCC | | | CGGGGGUUCC |
| | | | | | | TAATAAAG | | | UAAUAAAG |
| | | | | | | [SEQ ID | | | [SEQ ID |
| | | | | | | NO: | | | NO: |
| | | | | | | 2183] | | | 2184] |
| miR- | 967 | −2.64307 | 0.113083 | ACTGATGTAA | TGGCATATAC | GAGCTCAGTC | ACUGAUGUAA | UGGCAUAUAC | GAGCUCAGUC |
| 190a_ | | | | GTATATGAAC | TTACATCAAG | AAACCTGGAT | GUAUAUGAAC | UUACAUCAAG | AAACCUGGAU |
| M | | | | CA | [SEQ ID | GCCTTTTCTG | CA | [SEQ ID | GCCUUUUCUG |
| | | | | [SEQ ID | NO: | CAGGCGTCTG | [SEQ ID | NO: | CAGGCGUCUG |
| | | | | NO: | 1642] | TGACTGATGT | NO: | 1837] | UGACUGAUGU |
| | | | | 1619] | | AAGTATATGA | 1815] | | AAGUAUAUGA |
| | | | | | | ACCATGTTAT | | | ACCAUGUUAU |
| | | | | | | TTAATCCATG | | | UUAAUCCAUG |
| | | | | | | GCATATACTT | | | GCAUAUACUU |
| | | | | | | ACATCAAGCT | | | ACAUCAAGCU |
| | | | | | | ACAGTCTCTT | | | ACAGUCUCUU |
| | | | | | | GCCCTGTCTC | | | GCCCUGUCUC |
| | | | | | | CGGGGGTTCC | | | CGGGGGUUCC |
| | | | | | | TAATAAAG | | | UAAUAAAG |
| | | | | | | [SEQ ID | | | [SEQ ID |
| | | | | | | NO: | | | NO: |
| | | | | | | 1720] | | | 1917] |
| miR- | 3302 | −2.52395 | 0.249656 | TTGAACAAGG | CCCATCAGCC | GAGCTCAGTC | UUGAACAAGG | CCCAUCAGCC | GAGCUCAGUC |
| 190a_ | | | | GGCTGATTTG | CCTTGTTCCC | AAACCTGGAT | GGCUGAUUUG | CCUUGUUCCC | AAACCUGGAU |
| M | | | | GG | [SEQ ID | GCCTTTTCTG | GG | [SEQ ID | GCCUUUUCUG |
| | | | | [SEQ ID | NO: | CAGGCGTCTG | [SEQ ID | NO: | CAGGCGUCUG |
| | | | | NO: | 1643] | TGTTGAACAA | NO: | 1838] | UGUUGAACAA |
| | | | | 688] | | GGGGCTGATT | 1216] | | GGGGCUGAUU |
| | | | | | | TGGGTGTTAT | | | UGGGUGUUAU |
| | | | | | | TTAATCCACC | | | UUAAUCCACC |
| | | | | | | CATCAGCCCC | | | CAUCAGCCCC |
| | | | | | | TTGTTCCCCT | | | UUGUUCCCCU |
| | | | | | | ACAGTCTCTT | | | ACAGUCUCUU |
| | | | | | | GCCCTGTCTC | | | GCCCUGUCUC |
| | | | | | | CGGGGGTTCC | | | CGGGGGUUCC |
| | | | | | | TAATAAAG | | | UAAUAAAG |
| | | | | | | [SEQ ID | | | [SEQ ID |
| | | | | | | NO: | | | NO: |
| | | | | | | 1733] | | | 1930] |
| miR- | 2586 | −2.46486 | 0.014777 | TAGATTCAGA | CCATTCTACT | GAGCTCAGTC | UAGAUUCAGA | CCAUUCUACU | GAGCUCAGUC |
| 190a_ | | | | AGTAGAACTT | TCTGAATCCC | AAACCTGGAT | AGUAGAACUU | UCUGAAUCCC | AAACCUGGAU |
| M | | | | GG | [SEQ ID | GCCTTTTCTG | GG | [SEQ ID | GCCUUUUCUG |
| | | | | [SEQ ID | NO: | CAGGCGTCTG | [SEQ ID | NO: | CAGGCGUCUG |
| | | | | NO: | 1661] | TGTAGATTCA | NO: | 1856] | UGUAGAUUCA |
| | | | | 1621] | | GAAGTAGAAC | 1816] | | GAAGUAGAAC |
| | | | | | | TTGGTGTTAT | | | UUGGUGUUAU |
| | | | | | | TTAATCCACC | | | UUAAUCCACC |
| | | | | | | ATTCTACTTC | | | AUUCUACUUC |
| | | | | | | TGAATCCCCT | | | UGAAUCCCCU |
| | | | | | | ACAGTCTCTT | | | ACAGUCUCUU |
| | | | | | | GCCCTGTCTC | | | GCCCUGUCUC |
| | | | | | | CGGGGGTTCC | | | CGGGGGUUCC |
| | | | | | | TAATAAAG | | | UAAUAAAG |
| | | | | | | [SEQ ID | | | [SEQ ID |
| | | | | | | NO: | | | NO: |
| | | | | | | 1746] | | | 1943] |
| miR- | 3043 | −2.44458 | 0.289334 | TTTGGTGCAA | AGCGTTTGTT | GAGCTCAGTC | UUUGGUGCAA | AGCGUUUGUU | GAGCUCAGUC |
| 190a_ | | | | AACAAACAGG | TTGCACCACC | AAACCTGGAT | AACAAACAGG | UUGCACCACC | AAACCUGGAU |
| M | | | | CT | [SEQ ID | GCCTTTTCTG | CU | [SEQ ID | GCCUUUUCUG |
| | | | | [SEQ ID | NO: | CAGGCGTC | [SEQ ID | NO: | CAGGCGUCU |
| | | | | NO: | 1664] | TGTGTTTGGT | NO: | 1859] | GUGUUUGGUG |
| | | | | 1615] | | GCAAAACAAA | 1812] | | CAAAACAAAC |
| | | | | | | CAGGCTTGTT | | | AGGCUUGUUA |
| | | | | | | ATTTAATCCA | | | UUUAAUCCAA |
| | | | | | | AGCGTTTGTT | | | GCGUUUGUUU |
| | | | | | | TTGCACCACC | | | UGCACCACCC |
| | | | | | | CTACAGTCTC | | | UACAGUCUCU |
| | | | | | | TTGCCCTGTC | | | UGCCCUGUCU |
| | | | | | | TCCGGGGGTT | | | CCGGGGGUUC |
| | | | | | | CCTAATAAAG | | | CUAAUAAAG |
| | | | | | | [SEQ ID | | | [SEQ ID |
| | | | | | | NO: | | | NO: |
| | | | | | | 1750] | | | 1947] |
| miR- | 1755 | −2.34216 | 0.272299 | TCGGGTTGAA | CACTTCAGAT | GAGCTCAGTC | UCGGGUUGAA | CACUUCAGAU | GAGCUCAGUC |
| 190a_ | | | | ATCTGAAGTG | TTCAACCCAC | AAACCTGGAT | AUCUGAAGUG | UUCAACCCAC | AAACCUGGAU |
| M | | | | TG | [SEQ ID | GCCTTTTCTG | UG | [SEQ ID | GCCUUUUCUG |
| | | | | [SEQ ID | NO: | CAGGCGTCTG | [SEQ ID | NO: | CAGGCGUCUG |
| | | | | NO: | 1680] | TGTCGGGTTG | NO: | 1875] | UGUCGGGUUG |
| | | | | 657] | | AAATCTGAAG | 1185] | | AAAUCUGAAG |
| | | | | | | TGTGTGTTAT | | | UGUGUGUUAU |
| | | | | | | TTAATCCACA | | | UUAAUCCACA |
| | | | | | | CTTCAGATTT | | | CUUCAGAUUU |
| | | | | | | CAACCCACCT | | | CAACCCACCU |
| | | | | | | ACAGTCTCTT | | | ACAGUCUCUU |
| | | | | | | GCCCTGTCTC | | | GCCCUGUCUC |
| | | | | | | CGGGGGTTCC | | | CGGGGGUUCC |
| | | | | | | TAATAAAG | | | UAAUAAAG |
| | | | | | | [SEQ ID | | | [SEQ ID |
| | | | | | | NO: | | | NO: |
| | | | | | | 1778] | | | 1975] |
| miR- | 3269 | −2.3297 | 0.037293 | ACTGTAGGCA | CACAATATGT | GAGCTCAGTC | ACUGUAGGCA | CACAAUAUGU | GAGCUCAGUC |
| 190a_ | | | | ACATATTGCG | TGCCTACAAG | AAACCTGGAT | ACAUAUUGCG | UGCCUACAAG | AAACCUGGAU |
| M | | | | TG | [SEQ ID | GCCTTTTCTG | UG | [SEQ ID | GCCUUUUCUG |
| | | | | [SEQ ID | NO: | CAGGCGTCTG | [SEQ ID | NO: | CAGGCGUCUG |
| | | | | NO: | 1689] | TGACTGTAGG | NO: | 1884] | UGACUGUAGG |
| | | | | 1629] | | CAACATATTG | 1822] | | CAACAUAUUG |
| | | | | | | CGTGTGTTAT | | | CGUGUGUUAU |
| | | | | | | TTAATCCACA | | | UUAAUCCACA |
| | | | | | | CAATATGTTG | | | CAAUAUGUUG |
| | | | | | | CCTACAAGCT | | | CCUACAAGCU |
| | | | | | | ACAGTCTCTT | | | ACAGUCUCUU |
| | | | | | | GCCCTGTCTC | | | GCCCUGUCUC |
| | | | | | | CGGGGGTTCC | | | CGGGGGUUCC |
| | | | | | | TAATAAAG | | | UAAUAAAG |
| | | | | | | [SEQ ID | | | [SEQ ID |
| | | | | | | NO: | | | NO: |
| | | | | | | 1782] | | | 1979] |
| miR- | 3255 | −2.30348 | 0.418205 | ATTGCGTGGA | CCAAGCTTAC | GAGCTCAGTC | AUUGCGUGGA | CCAAGCUUAC | GAGCUCAGUC |
| 190a_ | | | | GTAAGCTGGT | TCCACGCACG | AAACCTGGAT | GUAAGCUGGU | UCCACGCACG | AAACCUGGAU |
| M | | | | GG | [SEQ ID | GCCTTTTCTG | GG | [SEQ ID | GCCUUUUCUG |
| | | | | [SEQ ID | NO: | CAGGCGTCTG | [SEQ ID | NO: | CAGGCGUCUG |
| | | | | NO: | 1697] | TGATTGCGTG | NO: | 1892] | UGAUUGCGUG |
| | | | | 617] | | GAGTAAGCTG | 306] | | GAGUAAGCUG |
| | | | | | | GTGGTGTTAT | (Same | | GUGGUGUUAU |
| | | | | | | TTAATCCACC | guide | | UUAAUCCACC |
| | | | | | | AAGCTTACT | as | | AAGCUUAC |
| | | | | | | CCACGCACGC | XD- | | UCCACGCACG |
| | | | | | | TACAGTCTCT | 14889) | | CUACAGUCUC |
| | | | | | | TGCCCTGTCT | | | UUGCCCUGUC |
| | | | | | | CCGGGGGTTC | | | UCCGGGGGUU |
| | | | | | | CTAATAAAG | | | CCUAAUAAAG |
| | | | | | | [SEQ ID | | | [SEQ ID |
| | | | | | | NO: | | | NO: |
| | | | | | | 1790] | | | 1987] |
| miR- | 3341 | −2.26383 | 0.394316 | ATAGACATGA | GTCAGCATCC | GAGCTCAGTC | AUAGACAUGA | GUCAGCAUCC | GAGCUCAGUC |
| 190a_ | | | | GGATGCTGAG | TCATGTCTCG | AAACCTGGAT | GGAUGCUGAG | UCAUGUCUCG | AAACCUGGAU |
| M | | | | AC | [SEQ ID | GCCTTTTCTG | AC | [SEQ ID | GCCUUUUCUG |
| | | | | [SEQ ID | NO: | CAGGCGTCTG | [SEQ ID | NO: | CAGGCGUCUG |
| | | | | NO: | 1703] | TGATAGACAT | NO: | 1898] | UGAUAGACAU |
| | | | | 1617] | | GAGGATGCTG | 1813] | | GAGGAUGCUG |
| | | | | | | AGACTGTTAT | | | AGACUGUUAU |
| | | | | | | TTAATCCAGT | | | UUAAUCCAGU |
| | | | | | | CAGCATCCTC | | | CAGCAUCCUC |
| | | | | | | ATGTCTCGCT | | | AUGUCUCGCU |
| | | | | | | ACAGTCTCTT | | | ACAGUCUCUU |
| | | | | | | GCCCTGTCTC | | | GCCCUGUCUC |
| | | | | | | CGGGGGTTCC | | | CGGGGGUUCC |
| | | | | | | TAATAAAG | | | UAAUAAAG |
| | | | | | | [SEQ ID | | | [SEQ ID |
| | | | | | | NO: | | | NO: |
| | | | | | | 1796] | | | 1993] |
| miR- | 1436 | −2.18684 | 0.218825 | TGAGTTATCT | GCCTAGAAAG | GAGCTCAGTC | UGAGUUAUCU | GCCUAGAAAG | GAGCUCAGUC |
| 190a_ | | | | CTTTCTAAGG | AGATAACTAC | AAACCTGGAT | CUUUCUAAGG | AGAUAACUAC | AAACCUGGAU |
| M | | | | GC | [SEQ ID | GCCTTTTCTG | GC | [SEQ ID | GCCUUUUCUG |
| | | | | [SEQ ID | NO: | CAGGCGTCTG | [SEQ ID | NO: | CAGGCGUCUG |
| | | | | NO: | 2185] | TGTGAGTTAT | NO: | 1899] | UGUGAGUUAU |
| | | | | 1632] | | CTCTTTCTAA | 1827] | | CUCUUUCUAA |
| | | | | | | GGGCTGTTAT | | | GGGCUGUUAU |
| | | | | | | TTAATCCAGC | | | UUAAUCCAGC |
| | | | | | | CTAGAAAGAG | | | CUAGAAAGAG |
| | | | | | | ATAACTACCT | | | AUAACUACCU |
| | | | | | | ACAGTCTCTT | | | ACAGUCUCUU |
| | | | | | | GCCCTGTCTC | | | GCCCUGUCUC |
| | | | | | | CGGGGGTTCC | | | CGGGGGUUCC |
| | | | | | | TAATAAAG | | | UAAUAAAG |
| | | | | | | [SEQ ID | | | [SEQ ID |
| | | | | | | NO: | | | NO: |
| | | | | | | 2186] | | | 2187] |
| miR- | 3330 | −2.17382 | 0.344749 | TATGCTGAGA | CCATTATCAG | GAGCTCAGTC | UAUGCUGAGA | CCAUUAUCAG | GAGCUCAGUC |
| 190a_ | | | | CTGATAATGT | TCTCAGCACC | AAACCTGGAT | CUGAUAAUGU | UCUCAGCACC | AAACCUGGAU |
| M | | | | GG | [SEQ ID | GCCTTTTCTG | GG | [SEQ ID | GCCUUUUCUG |
| | | | | [SEQ ID | NO: | CAGGCGTCTG | [SEQ ID | NO: | CAGGCGUCUG |
| | | | | NO: | 1634] | TGTATGCTGA | NO: | 1829] | UGUAUGCUGA |
| | | | | 1614] | | GACTGATAAT | 1811] | | GACUGAUAAU |
| | | | | | | GTGGTGTTAT | | | GUGGUGUUAU |
| | | | | | | TTAATCCACC | | | UUAAUCCACC |
| | | | | | | ATTATCAGTC | | | AUUAUCAGUC |
| | | | | | | TCAGCACCCT | | | UCAGCACCCU |
| | | | | | | ACAGTCTCTT | | | ACAGUCUCUU |
| | | | | | | GCCCT | | | GCCC |
| | | | | | | GTCTCCGGGG | | | UGUCUCCGGG |
| | | | | | | GTTCCTAATA | | | GGUUCCUAAU |
| | | | | | | AAG | | | AAAG |
| | | | | | | [SEQ ID | | | [SEQ ID |
| | | | | | | NO: | | | NO: |
| | | | | | | 2188] | | | 2189] |
| miR | 3302 | −2.42428 | 0.285147 | TTGAACAAGG | CCCAAATCGC | CCTGGAGGCT | UUGAACAAGG | CCCAAAUCGC | CCUGGAGGCU |
| 155-M | | | | GGCTGATTTG | CCTTGTTCAA | TGCTGAAGGC | GGCUGAUUUG | CCUUGUUCAA | UGCUGAAGGC |
| | | | | GG | [SEQ ID | TGTATGCTGT | GG | [SEQ ID | UGUAUGCUGU |
| | | | | [SEQ ID | NO: | TGAACAAGGG | [SEQ ID | NO: | UGAACAAGGG |
| | | | | NO: | 1652] | GCTGATTTGG | NO: | 1847] | GCUGAUUUGG |
| | | | | 688] | | GTTTTGGCCA | 1216] | | GUUUUGGCCA |
| | | | | | | CTGACTGACC | | | CUGACUGACC |
| | | | | | | CAAATCGCCC | | | CAAAUCGCCC |
| | | | | | | TTGTTCAACA | | | UUGUUCAACA |
| | | | | | | GGACACAAGG | | | GGACACAAGG |
| | | | | | | CCTGTTACTA | | | CCUGUUACUA |
| | | | | | | GCACTCACAT | | | GCACUCACAU |
| | | | | | | GGAACAAATG | | | GGAACAAAUG |
| | | | | | | GCCACCGG | | | GCCACCGG |
| | | | | | | [SEQ ID | | | [SEQ ID |
| | | | | | | NO: | | | NO: |
| | | | | | | 1758] | | | 1955] |
| miR | 2586 | −2.4228 | −0.13209 | TAGATTCAGA | CCAAGTTCAC | CCTGGAGGCT | UAGAUUCAGA | CCAAGUUCAC | CCUGGAGGCU |
| 155-M | | | | AGTAGAACTT | TCTGAATCTA | TGCTGAAGGC | AGUAGAACUU | UCUGAAUCUA | UGCUGAAGGC |
| | | | | GG | [SEQ ID | TGTATGCTGT | GG | [SEQ ID | UGUAUGCUGU |
| | | | | [SEQ ID | NO: | AGATTCAGAA | [SEQ ID | NO: | AGAUUCAGAA |
| | | | | NO: | 1653] | GTAGAACTTG | NO: | 1848] | GUAGAACUUG |
| | | | | 1621] | | GTTTTGGCCA | 1816] | | GUUUUGGCCA |
| | | | | | | CTGACTGACC | | | CUGACUGACC |
| | | | | | | AAGTTCACTC | | | AAGUUCACUC |
| | | | | | | TGAATCTACA | | | UGAAUCUACA |
| | | | | | | GGACACAAGG | | | GGACACAAGG |
| | | | | | | CCTGTTACTA | | | CCUGUUACUA |
| | | | | | | GCACTCACAT | | | GCACUCACAU |
| | | | | | | GGAACAAATG | | | GGAACAAAUG |
| | | | | | | GCCACCGG | | | GCCACCGG |
| | | | | | | [SEQ ID | | | [SEQ ID |
| | | | | | | NO: | | | NO: |
| | | | | | | 1759] | | | 1956] |
| miR | 1436 | −2.22884 | 0.141256 | TGAGTTATCT | GCCCTTAGAA | CCTGGAGGCT | UGAGUUAUCU | GCCCUUAGAA | CCUGGAGGCU |
| 155-M | | | | CTTTCTAAGG | GGATAACTCA | TGCTGAAGGC | CUUUCUAAGG | GGAUAACUCA | UGCUGAAGGC |
| | | | | GC | [SEQ ID | TGTATGCTGT | GC | [SEQ ID | UGUAUGCUGU |
| | | | | [SEQ ID | NO: | GAGTTATCTC | [SEQ ID | NO: | GAGUUAUCUC |
| | | | | NO: | 1710] | TTTCTAAGGG | NO: | 1900] | UUUCUAAGGG |
| | | | | 1632] | | CTTTTGGCCA | 1827] | | CUUUUGGCCA |
| | | | | | | CTGACTGAGC | | | CUGACUGAGC |
| | | | | | | CCTTAGAAGG | | | CCUUAGAAGG |
| | | | | | | ATAACTCACA | | | AUAACUCACA |
| | | | | | | GGACACAAGG | | | GGACACAAGG |
| | | | | | | CCTGTTACTA | | | CCUGUUACUA |
| | | | | | | GCACTCACAT | | | GCACUCACAU |
| | | | | | | GGAA | | | GGAACAAAUG |
| | | | | | | CAAATGGCCA | | | GCCACCGG |
| | | | | | | CCGG | | | [SEQ ID |
| | | | | | | [SEQ ID | | | NO: |
| | | | | | | NO: | | | 2007] |
| | | | | | | 1810] | | | |
| miR | 3272 | −2.02279 | −0.09047 | AGGACTGTAG | GCAATATGTG | CCTGGAGGCT | AGGACUGUAG | GCAAUAUGUG | CCUGGAGGCU |
| 155-M | | | | GCAACATATT | CTACAGTCCT | TGCTGAAGGC | GCAACAUAUU | CUACAGUCCU | UGCUGAAGGC |
| | | | | GC | [SEQ ID | TGTATGCTGA | GC | [SEQ ID | UGUAUGCUGA |
| | | | | [SEQ ID | NO: | GGACTGTAGG | [SEQ ID | NO: | GGACUGUAGG |
| | | | | NO: | 1687] | CAACATATTG | NO: | 1882] | CAACAUAUUG |
| | | | | 1618] | | CTTTTGGCCA | 1814] | | CUUUUGGCCA |
| | | | | | | CTGACTGAGC | | | CUGACUGAGC |
| | | | | | | AATATGTGCT | | | AAUAUGUGCU |
| | | | | | | ACAGTCCTCA | | | ACAGUCCUCA |
| | | | | | | GGACACAAGG | | | GGACACAAGG |
| | | | | | | CCTGTTACTA | | | CCUGUUACUA |
| | | | | | | GCACTCACAT | | | GCACUCACAU |
| | | | | | | GGAACAAATG | | | GGAACAAAUG |
| | | | | | | GCCACCGG | | | GCCACCGG |
| | | | | | | [SEQ ID | | | [SEQ ID |
| | | | | | | NO: | | | NO: |
| | | | | | | 2190] | | | 2191] |
| miR | 1755 | −1.98201 | −0.05599 | TCGGGTTGAA | CACACTTCGA | CCTGGAGGCT | UCGGGUUGAA | CACACUUCGA | CCUGGAGGCU |
| 155-M | | | | ATCTGAAGTG | TTCAACCCGA | TGCTGAAGGC | AUCUGAAGUG | UUCAACCCGA | UGCUGAAGGC |
| | | | | TG | [SEQ ID | TGTATGCTGT | UG | [SEQ ID | UGUAUGCUGU |
| | | | | [SEQ ID | NO: | CGGGTTGAAA | [SEQ ID | NO: | CGGGUUGAAA |
| | | | | NO: | 1676] | TCTGAAGTGT | NO: | 1871] | UCUGAAGUGU |
| | | | | 657] | | GTTTTGGCCA | 1185] | | GUUUUGGCCA |
| | | | | | | CTGACTGACA | | | CUGACUGACA |
| | | | | | | CACTTCGATT | | | CACUUCGAUU |
| | | | | | | CAACCCGACA | | | CAACCCGACA |
| | | | | | | GGACACAAGG | | | GGACACAAGG |
| | | | | | | CCTGTTACTA | | | CCUGUUACUA |
| | | | | | | GCACTCACAT | | | GCACUCACAU |
| | | | | | | GGAACAAATG | | | GGAACAAAUG |
| | | | | | | GCCACCGG | | | GCCACCGG |
| | | | | | | [SEQ ID | | | [SEQ ID |
| | | | | | | NO: | | | NO: |
| | | | | | | 2192] | | | 2193] |
| miR | 1162 | −1.86763 | −0.12823 | AACTGTACCA | CAGACTTTTT | CCTGGAGGCT | AACUGUACCA | CAGACUUUUU | CCUGGAGGCU |
| 155-M | | | | CAACAAAGTC | GGGTACAGTT | TGCTGAAGGC | CAACAAAGUC | GGGUACAGUU | UGCUGAAGGC |
| | | | | TG | [SEQ ID | TGTATGCTGA | UG | [SEQ ID | UGUAUGCUGA |
| | | | | [SEQ ID | NO: | ACTGTACCAC | [SEQ ID | NO: | ACUGUACCAC |
| | | | | NO: | 1706] | AACAAAGTCT | NO: | 1905] | AACAAAGUCU |
| | | | | 652] | | GTTTTGGCCA | 1180] | | GUUUUGGCCA |
| | | | | | | CTGACTGACA | | | CUGACUGACA |
| | | | | | | GACTTTTTGG | | | GACUUUUUGG |
| | | | | | | GTACAGTTCA | | | GUACAGUUCA |
| | | | | | | GGACACAAGG | | | GGACACAAGG |
| | | | | | | CCTGTTACTA | | | CCUGUUACUA |
| | | | | | | GCACTCACAT | | | GCACUCACAU |
| | | | | | | GGAA | | | GGAACAAAUG |
| | | | | | | CAAATGGCCA | | | GCCACCGG |
| | | | | | | CCGG | | | [SEQ ID |
| | | | | | | [SEQ ID | | | NO: |
| | | | | | | NO: | | | 2195] |
| | | | | | | 2194] | | | |
| miR | 1580 | −1.73791 | −0.21175 | ACTGGAATTT | AGCAGTTCGA | CCTGGAGGCT | ACUGGAAUUU | AGCAGUUCGA | CCUGGAGGCU |
| 155-M | | | | CTCTGAACTG | GAATTCCAGT | TGCTGAAGGC | CUCUGAACUG | GAAUUCCAGU | UGCUGAAGGC |
| | | | | CT | [SEQ ID | TGTATGCTGA | CU | [SEQ ID | UGUAUGCUGA |
| | | | | [SEQ ID | NO: | CTGGAATTTC | [SEQ ID | NO: | CUGGAAUUUC |
| | | | | NO: | 2171] | TCTGAACTGC | NO: | 2173] | UCUGAACUGC |
| | | | | 1622] | | TTTTTGGCCA | 1817] | | UUUUUGGCCA |
| | | | | | | CTGACTGAAG | | | CUGACUGAAG |
| | | | | | | CAGTTCGAGA | | | CAGUUCGAGA |
| | | | | | | ATTCCAGTCA | | | AUUCCAGUCA |
| | | | | | | GGACACAAGG | | | GGACACAAGG |
| | | | | | | CCTGTTACTA | | | CCUGUUACUA |
| | | | | | | GCACTCACAT | | | GCACUCACAU |
| | | | | | | GGAACAAATG | | | GGAACAAAUG |
| | | | | | | GCCACCGG | | | GCCACCGG |
| | | | | | | [SEQ ID | | | [SEQ ID |
| | | | | | | NO: | | | NO: |
| | | | | | | 2196] | | | 2197] |
| miR | 3273 | −1.30321 | −0.03402 | TAGGACTGTA | CAATATGTGC | CCTGGAGGCT | UAGGACUGUA | CAAUAUGUGC | CCUGGAGGCU |
| 155-M | | | | GGCAACATAT | CACAGTCCTA | TGCTGAAGGC | GGCAACAUAU | CACAGUCCUA | UGCUGAAGGC |
| | | | | TG | [SEQ ID | TGTATGCTGT | UG | [SEQ ID | UGUAUGCUGU |
| | | | | [SEQ ID | NO: | AGGACTGTAG | [SEQ ID | NO: | AGGACUGUAG |
| | | | | NO: | 1683] | GCAACATATT | NO: | 1878] | GCAACAUAUU |
| | | | | 1628] | | GTTTTGGCCA | 1821] | | GUUUUGGCCA |
| | | | | | | CTGACTGACA | | | CUGACUGACA |
| | | | | | | ATATGTGCCA | | | AUAUGUGCCA |
| | | | | | | CAGTCCTACA | | | CAGUCCUACA |
| | | | | | | GGACACAAGG | | | GGACACAAGG |
| | | | | | | CCTGTTACTA | | | CCUGUUACUA |
| | | | | | | GCACTCACAT | | | GCACUCACAU |
| | | | | | | GGAACAAATG | | | GGAACAAAUG |
| | | | | | | GCCACCGG | | | GCCACCGG |
| | | | | | | [SEQ ID | | | [SEQ ID |
| | | | | | | NO: | | | NO: |
| | | | | | | 2198] | | | 2199] |
| miR | 3291 | −1.28366 | 0.074799 | TCTGATTTGG | CCTCAGCATT | CCTGGAGGCT | UCUGAUUUGG | CCUCAGCAUU | CCUGGAGGCU |
| 155-M | | | | GAACTGCTGA | CCAAATCAGA | TGCTGAAGGC | GAACUGCUGA | CCAAAUCAGA | UGCUGAAGGC |
| | | | | GG | [SEQ ID | TGTATGCTGT | GG | [SEQ ID | UGUAUGCUGU |
| | | | | [SEQ ID | NO: | CTGATTTGGG | [SEQ ID | NO: | CUGAUUUGGG |
| | | | | NO: | 2201] | AACTGCTGAG | NO: | 2204] | AACUGCUGAG |
| | | | | 2200] | | GTTTTGGCCA | 2203] | | GUUUUGGCCA |
| | | | | | | CTGACTGACC | | | CUGACUGACC |
| | | | | | | TCAGCATTCC | | | UCAGCAUUCC |
| | | | | | | AAATCAGACA | | | AAAUCAGACA |
| | | | | | | GGACACAAGG | | | GGACACAAGG |
| | | | | | | CCTGTTACTA | | | CCUGUUACUA |
| | | | | | | GCACTCACAT | | | GCACUCACAU |
| | | | | | | GGA | | | GGAACAAAUG |
| | | | | | | ACAAATGGCC | | | GCCACCGG |
| | | | | | | ACCGG | | | [SEQ ID |
| | | | | | | [SEQ ID | | | NO: |
| | | | | | | NO: | | | 2205] |
| | | | | | | 2202] | | | |
| miR | 2914 | −1.18865 | 0.250219 | TGTGGGTTGA | ATGCAAAGAG | CCTGGAGGCT | UGUGGGUUGA | AUGCAAAGAG | CCUGGAGGCU |
| 155-M | | | | ACTCCTTTGC | TCAACCCACA | TGCTGAAGGC | ACUCCUUUGC | UCAACCCACA | UGCUGAAGGC |
| | | | | AT | [SEQ ID | TGTATGCTGT | AU | [SEQ ID | UGUAUGCUGU |
| | | | | [SEQ ID | NO: | GTGGGTTGAA | [SEQ ID | NO: | GUGGGUUGAA |
| | | | | NO: | 2207] | CTCCTTTGCA | NO: | 2210] | CUCCUUUGCA |
| | | | | 2206] | | TTTTTGGCCA | 2209] | | UUUUUGGCCA |
| | | | | | | CTGACTGAAT | | | CUGACUGAAU |
| | | | | | | GCAAAGAGTC | | | GCAAAGAGUC |
| | | | | | | AACCCACACA | | | AACCCACACA |
| | | | | | | GGACACAAGG | | | GGACACAAGG |
| | | | | | | CCTGTTACTA | | | CCUGUUACUA |
| | | | | | | GCACTCACAT | | | GCACUCACAU |
| | | | | | | GGAACAAATG | | | GGAACAAAUG |
| | | | | | | GCCACCGG | | | GCCACCGG |
| | | | | | | [SEQ ID | | | [SEQ ID |
| | | | | | | NO: | | | NO: |
| | | | | | | 2208] | | | 2211] |

### Methods

### Oligo Pool Design and Synthesis:

A total of 7500 elements of 210 bp length were designed for synthesis, split approximately evenly across 20 miRNA backbones. There were more elements in the miR-1-1, miR-155, and miR-16-2 backbones as elements that had been tested in arrayed experiments were also included in this screen. ATXN2 targeting sequences accounted for about 60% of the library.

Each element included the 138 nt pri-miRNA, flanked by dual 18 nt adapter pairs. The outer adapter pair was miR-specific and the inside adapter pair was universal.

### Full DS2 Library Cloning Strategy

Oligonucleotide pools were synthesized (Twist Bioscience) and were reconstituted in nuclease free water. For cloning the EF1A oligo pool into pLVX_EF1A-MCS-WPRE-CMV-Puro, the vector was first linearized by XbaI and EcoRI restriction digest and gel purified. The primers DS2_EF1A_fw and DS2_EF1A_rv were used to amplify the oligo pool through 10 cycles of PCR and purified. The purified pooled insert and purified linearized vector were assembled with NEB HiFi assembly, precipitated, concentrated, and electroporated into Lucigen Endura electrocompetent cells, recovered and maxiprepped. Oligo pools were PCR amplified with the following conditions.

The PCR mix consisted of:

| Component | Volume (ul) |
|---|---|
| NEBNext 2 × mix (M0541L) | 50 |
| DMSO (D9170-5VL) | 2 |
| Betaine (Sigma, B0300-1VL) | 10 |
| 100uM FW primer | 0.5 |
| (DS2_EF1A_fw) | |
| 100uM RV primer | 0.5 |
| (DS2_EF1A_rv) | |
| 1ng EF1A oligo pool in | 37 |
| nuclease free water | |
| Total | 100 |

The PCR cycling parameters were:

| STEP | TEMP | TIME | |
|---|---|---|---|
| Initial Denaturation | 98° C. | 30 | s |
| 10 cycles | 98° C. | 10 | s |
| | 64° C. | 30 | s |
| | 72° C. | 15 | s |
| Final Extension | 72° C. | 2 | min |
| Hold | 4-10° C. | | |

PCR products of 210 bp length were purified by agarose gel extraction (Zymoclean gel DNA recovery kit, D4002). Agilent Tapestation High Sensitivity D1000 was used to quantify the molarity of the 210 bp peak and to confirm removal of contaminating bands.

HiFi assembly of the pooled library was performed by assembling at 5 to 1 insert to backbone molar ratio. 15 ul of 2×HiFi assembly master mix (NEB, E2621L) and 15 ul of insert and backbone (about 0.375 pmol purified miR library insert to 0.075 pmol purified backbone) and incubating for 1 hr at 50° C.

Assembled DNA was precipitated by adding 1 ul of 20 mg/mL glycogen, one-tenth volume of 3M sodium acetate pH 5.5, and 2.2× volume of ethanol, mixed and stored overnight at −80° C.

Samples were spun at 16,000×g for 15 min at 4° C. Supernatant was removed and discarded. Pellets were washed twice with 1 ml of 70% ice cold ethanol and let to dry, then dissolved in 4 ul nuclease free water.

Purified DNA and 0.1 cm Gene Pulser Cuvettes (Bio-rad, 165-2089) were placed on ice for 10 min. 50 ul of Lucigen Endura electrocompetent cells were thawed briefly on ice. 4 ul of precipitated HiFi reaction was added to 25 ul of electrocompetent cells, mixed, and transferred to the cuvette. DNA and cell mixes were electroporated with the following parameters: 1800 Volts, 10 uF, 600 Ohms, 0.1 cm cuvette. Cuvettes were immediately flushed twice with 1 mL Lucigen recovery media. Cells were recovered at 37° C. for 1 hour at 230 rpm.

To titer the transformed bacteria, 2 uL of each culture was diluted into 200 uL of LB and 100 ul or 10 ul of this plated at a 1:100 dilution onto LB agar plates plus appropriate antibiotic. The number of colonies were counted the next day to determine total number of transformants.

Liquid cultures were inoculated into the appropriate amount of LB with antibiotic for maxi prep. Pooled plasmid libraries were prepared with a Qiagen Plasmid Maxi Kit following the manufacturer's instructions.

### Preparation and Titering of Pooled EF1A Library

Lentivirus was produced with the Takara packaging plasmid system in Lenti-X 293T cells. Functional titers were determined by Cell Titer Glo following infection and puromycin selection for 3 days to identify conditions to achieve MOI=0.1.

### Execution of Full Library Screens for Atxn2 Levels and Dropout

Concurrent ATXN2 levels and dropout screens were conducted similarly to DS1. U2OS cells were infected at day 0 with the lentiviral pooled EF1A library at 2000× coverage and MOI=0.1.

For the dropout screen, a T0 baseline sample was collected at day 1. Puromycin was added on day 2 and MOI was confirmed by plating cells for Cell Titer Glo titer assessment at day 5. After day 7, puromycin was removed and cells were passaged at a minimum of 20 million cells to day 18, upon which the T1 final cell population was collected.

For the ATXN2 protein levels screen, on day 7 cells were harvested and fixed in 6% sucrose/8% PFA for 10-15 min at room temperature, centrifuged 600×g for 3 minutes, washed thrice using the permeabilization buffer (eBioscience, 00-5523-00), mixed with wash buffer and incubated for 15-20 min at room temperature. Anti-ATXN2 primary antibody (1:200, BD, 611378) was incubated for 30-60 min at RT. Cells were washed thrice and AF647 secondary (1:200, Biolegend, 405322) was added and incubated for 45 min. After three washes, cells were resuspended in FACS buffer and sorted on a BD FACSAria Fusion. After gating for singlets, 25% high and low Atxn2 gates were drawn, adjusting for cell size by sorting on an APC/SSC ratiometric gate. Once 3-3.5 million cells were collected for the 25% high and low sort gates, remaining cells were sorted on a 10% low gate (1 million cells collected) to further enrich for high performing guides. The reference population was collected by sorting for singlets.

Fixed populations of sorted cells were decrosslinked with 1% SDS/1% sodium bicarbonate and incubated overnight at 65 C. Genomic DNA was extracted with Machery Nagel NucleoSpin L kit and proceeded to nested PCR to prepare sequencing libraries.

### Sequencing Library Preparation

Nested PCR was performed to produce Illumina adapted sequencing amplicons. The first PCR reaction was performed on all genomic DNA extracted from each cell pellet. A maximum of 5 ug genomic DNA was used per 100 ul PCR reaction using the conditions listed below.

| Component | Volume (ul) |
|---|---|
| NEBNext 2 × mix (M0541L) | 50 |
| DMSO (D9170-5VL) | 2 |
| Betaine (Sigma, B0300-1VL) | 10 |
| 100uM FW primer | 0.5 |
| (EF1A_F_intron) | |
| 100uM RV primer (WPRE_R_ CG) | 0.5 |
| 5ug genomic DNA and nuclease | 37 |
| free water | |
| Total | 100 |

| STEP | TEMP | | TIME | |
|---|---|---|---|---|
| Initial Denaturation | 98° | C. | 30 | s |
| 20 cycles | 98° | C. | 10 | s |
| | 64 | C. | 30 | s |
| | 72° | C. | 20 | s |
| Final Extension | 72° | C. | 2 | minutes |
| Hold | 4-10° | C. | | |

Following PCR, all reactions from a given sample were consolidated into a single tube.

Bead purification of the first PCR product of 564 bp expected size was performed with 0.5× and 0.9× double sided SPRI bead ratios. Specifically, 25 ul of SPRIselect (Beckman, B23318) was added to 50 ul first PCR product, mixed well by pipetting, and incubated at room temperature for 10 min. Samples were placed on a magnetic stand for 5 min. The supernatant was transferred to a new tube. 45 ul SPRIselect was added to the transferred supernatant, mixed well by pipetting, and incubated at room temperature for 10 min. Samples were placed on a magnetic stand for 5 min. Supernatant was then removed. Beads were washed twice with 1 ml fresh 80% ethanol over 2 min incubations. Beads with bound DNA were air dried for 5-10 min and eluted with 20 ul elution buffer from the Machery Nagel kit.

A second PCR was performed to add sample barcodes and Illumina adapters with the following conditions:

| Component | Volume (ul) |
|---|---|
| NEBNext 2 × mix (M0541L) | 50 |
| DMSO (D9170-5VL) | 2 |
| Betaine (Sigma, B0300-1VL) | 10 |
| 100uM FW primer (P5-DS2-FW) | 0.5 |
| 100uM RV primer | 0.5 |
| (RV primer P7-DS2-RV-1 to 12 for | |
| multiplexing onto MiSeq run) | |
| 1^{st} PCR bead purification product | 5 |
| Nuclease free water | 32 |
| Total | 100 |

| STEP | TEMP | | TIME | |
|---|---|---|---|---|
| Initial Denaturation | 98° | C. | 30 | s |
| 10 cycles | 98° | C. | 10 | s |
| | 64 | C | 30 | s |
| | 72° | C. | 20 | s |
| Final Extension | 72° | C. | 2 | minutes |
| Hold | 4-10° | C. | | |

Bead purification of the second PCR product with 300 bp expected size was performed with 0.7× and 1.2× double sided SPRI bead ratios. Specifically, 35 ul of SPRIselect (Beckman, B23318) was added to 50 ul first PCR product, mixed well by pipetting, and incubated at room temperature for 10 min. Samples were placed on a magnetic stand for 5 min. The supernatant was transferred to a new tube. 60 ul SPRIselect was added to the transferred supernatant, mixed well by pipetting, and incubated at room temperature for 10 min. Samples were placed on a magnetic stand for 5 min. Supernatant was then removed. Beads were washed twice with 1 ml fresh 80% ethanol over 2 min incubations. Beads with bound DNA were air dried for 5-10 min and eluted with 20 ul elution buffer from the Machery Nagel kit.

Final bead purified 2^{nd} PCR product was quantified by Tapestation High Sensitivity D1000 (Agilent) and multiplexed at equimolar ratio for sequencing on a MiSeq (Illumina). Using manufacturer's protocols, 15 pM libraries were denatured and mixed with 2% PhiX control. DS2-EF1A-READ1 primer was spiked into position 12 of the MiSeq v3 cartridge (Illumina). Read 1 was set to 139 cycles and index reads was set to 6 cycles.

Data were demultiplexed using the fastq generation module and analyzed.

### Primers

| Name | Sequence |
|---|---|
| WPRE_R_CG | Catagcgtaaaaggagcaaca |
| | (SEQ ID NO: 628) |
| EF1A_F_ | Ccaggcacctcgattagttc |
| intron | (SEQ ID NO: 2212) |
| DS2-EF1A- | AAGTAAGcctgcaggAATTgCCTAGGgt |
| READ1 | (SEQ ID NO: 2213) |
| P5-DS2-FW | aatgatacggcgaccaccgagatctaca |
| | cAAGTAAGcctgcaggAATTgCCTAGGg |
| | t |
| | (SEQ ID NO: 2214) |
| P7-DS2- | CAAGCAGAAGACGGCATACGAGATCTTG |
| RV_1 | TAGTGACTGGAGTTCAGACGTGTGCTCT |
| | TCCGATCTACATGtctcgacctggctta |
| | ctagtG |
| | (SEQ ID NO: 2215) |
| P7-DS2- | CAAGCAGAAGACGGCATACGAGATGCCA |
| RV_2 | ATGTGACTGGAGTTCAGACGTGTGCTCT |
| | TCCGATCTACATGtctcgacctggctta |
| | ctagtG |
| | (SEQ ID NO: 2216) |
| P7-DS2- | CAAGCAGAAGACGGCATACGAGATAGTT |
| RV_3 | CCGTGACTGGAGTTCAGACGTGTGCTCT |
| | TCCGATCTACATGtctcgacctggctta |
| | ctagtG |
| | (SEQ ID NO: 2217) |
| P7-DS2- | CAAGCAGAAGACGGCATACGAGATTAGC |
| RV_4 | TTGTGACTGGAGTTCAGACGTGTGCTCT |
| | TCCGATCTACATGtctcgacctggctta |
| | ctagtG |
| | (SEQ ID NO: 2218) |
| P7-DS2- | CAAGCAGAAGACGGCATACGAGATTTAG |
| RV_5 | GCGTGACTGGAGTTCAGACGTGTGCTCT |
| | TCCGATCTACATGtctcgacctggctta |
| | ctagtG |
| | (SEQ ID NO: 2219) |
| P7-DS2- | CAAGCAGAAGACGGCATACGAGATATCA |
| RV_6 | CGGTGACTGGAGTTCAGACGTGTGCTCT |
| | TCCGATCTACATGtctcgacctggctta |
| | ctagtG |
| | (SEQ ID NO: 2220) |
| P7-DS2- | CAAGCAGAAGACGGCATACGAGATGAGT |
| RV_7 | GGGTGACTGGAGTTCAGACGTGTGCTCT |
| | TCCGATCTACATGtctcgacctggctta |
| | ctagtG |
| | (SEQ ID NO: 2221) |
| P7-DS2- | CAAGCAGAAGACGGCATACGAGATAGTC |
| RV_8 | AAGTGACTGGAGTTCAGACGTGTGCTCT |
| | TCCGATCTACATGtctcgacctggctta |
| | ctagtG |
| | (SEQ ID NO: 2222) |
| P7-DS2- | CAAGCAGAAGACGGCATACGAGATACAG |
| RV_9 | TGGTGACTGGAGTTCAGACGTGTGCTCT |
| | TCCGATCTACATGtctcgacctggctta |
| | ctagtG |
| | (SEQ ID NO: 2223) |
| P7-DS2- | CAAGCAGAAGACGGCATACGAGATTGAC |
| RV_10 | CAGTGACTGGAGTTCAGACGTGTGCTCT |
| | TCCGATCTACATGtctcgacctggctta |
| | ctagtG |
| | (SEQ ID NO: 2224) |
| P7-DS2- | CAAGCAGAAGACGGCATACGAGATCAGA |
| RV_11 | TCGTGACTGGAGTTCAGACGTGTGCTCT |
| | TCCGATCTACATGtctcgacctggctta |
| | ctagtG |
| | (SEQ ID NO: 2225) |
| P7-DS2- | CAAGCAGAAGACGGCATACGAGATGGCT |
| RV_12 | ACGTGACTGGAGTTCAGACGTGTGCTCT |
| | TCCGATCTACATGtctcgacctggctta |
| | ctagtG |
| | (SEQ ID NO: 2226) |

### Example 6: Evaluation of miR Backbones in AAV Plasmids

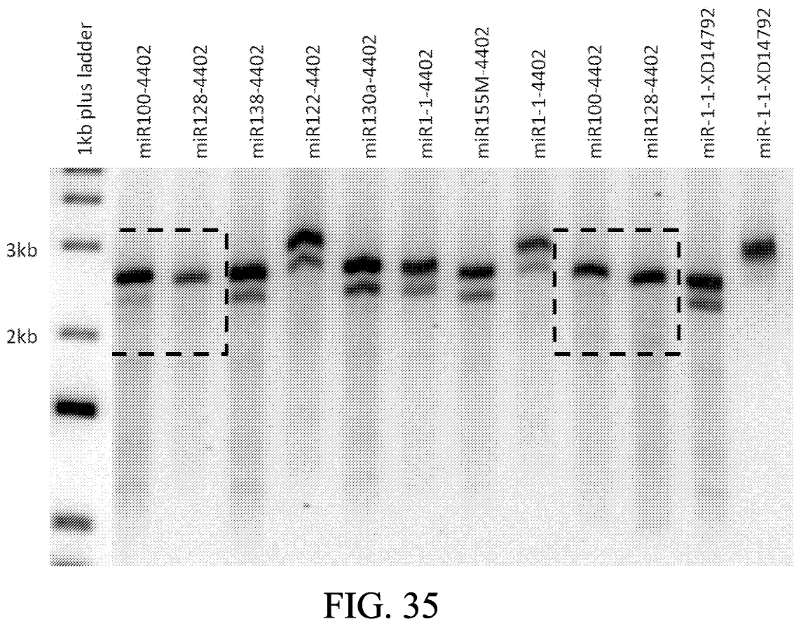

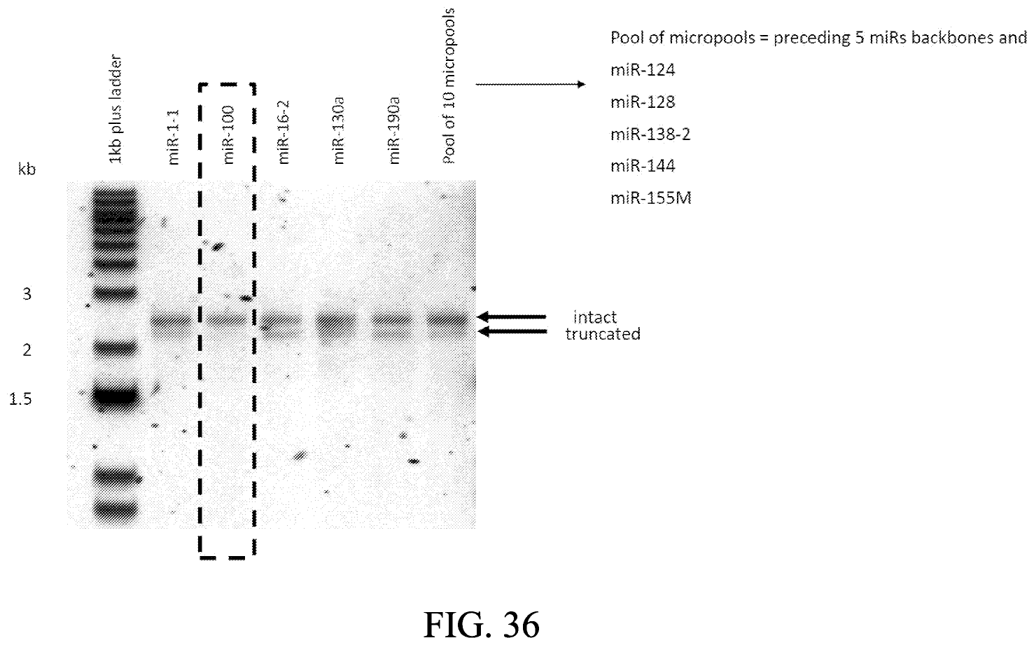

A subset of these miR backbones were subsequently evaluated in cis plasmids for AAV production. As described in Example 4 for AAV packaging of miR-16-2 backbone containing amiRNA vectors, cis plasmids containing an H1 promoter (nucleotides 113-203 of SEQ ID NO:1522) and a stuffer sequence (“AMELY_ITR_Stuffer_V1”—nucleotides 348-2228 of SEQ ID NO:1522) and various miR backbones were used to package AAV, and then the uniformity of vector genomes produced was assessed by agarose gel electrophoresis. SEQ ID NO:1522 provides an example of such a sequence from 5′ ITR to 3′ ITR, where for each library element the plasmid would be as shown but with the bases denoted with ‘n’ in the miR backbone insert (nucleotides 204-341 of SEQ ID NO:1522) replaced by the appropriate 138-bp artificial miRNA sequence (backbone, guide, and passenger insert. FIG. 35 shows the indicated set of AAVs, with indicated ATXN2 guide sequence (targeting position 4402 in ATXN2 transcript, —SEQ ID NO:1279 (RNA)), and overall miR cassette sequences constructed from the rules in Table 8. Among the miR backbones assessed, miR-100 and miR-128 backbone-embedded miRs had more uniform gel patterns. To more generally assess the vector integrity of AAV containing different miR backbones, libraries of cis plasmids, each containing the complete set of ATXN2 targeting amiRNA guide sequences as in Deep Screen 2, were used to package AAV as before. The oligonucleotide amplification strategy used in this experiment does not distinguish between parent and “_M” forms of the miR backbones where both were originally present in the Deep Screen 2 library, so the libraries include mixtures of, for example, miR-100 and miR-100_M backbone containing elements; miR-1-1 and mir-1-1_M backbones. FIG. 36 shows that, as with AAVs containing a miR-100 backbone and the specific guide sequence 4402 (SEQ ID NO:751 (DNA); SEQ ID NO:1279 (RNA)), AAVs derived from a library of miRs embedded in the miR-100 and miR-100_M backbones exhibit a more uniform gel electrophoresis pattern than AAVs with other miR backbones. Although the specific composition of the cis plasmid libraries was not assessed after packaging and confirmed to be consistent across libraries with different miR backbones, the simplest interpretation of this data is that on average, across a range of specific miRs, AAV vector genomes with a miR-100 backbone exhibit more uniform, full-length size, than other backbones.

Based on the combined properties of good knockdown performance and good AAV vector genome uniformity, miR-100 and the slightly modified miR-100_M were prioritized as backbones for advancement. ‘Micropool’ plasmid libraries comprising amiRNAs inserted into unpackaged AAV cis plasmid scAAV_AMELY_V1_H1 (SEQ ID NO:1522; amiRNA insert located at nucleotides 204-341) were tested by transfecting plasmid library into HEK293T cells and harvesting small RNA. As above the oligonucleotide amplification strategy to construct the plasmid library did not distinguish between the miR100 and miR100_M backbones, and so the library represents a mix of both; however, given the similar performance overall of miRs from parent and _M form backbones, the mix of backbones in the library is unlikely to degrade the overall ability to detect precisely processed miRNAs. This small RNAseq data was integrated to evaluate processing precision of individual amiRNAs within the library, as in the below examples.

### Methods

### AAV Micropool Cloning

To clone micropools into the scAAV_AMELY_V1_H1 backbone (to yield plasmids as set forth in SEQ ID NO:1522), the backbone was first linearized by AarI digestion of a cloning site region and agarose gel purified.

Micropools were amplified using the following conditions, using miR-1-1 as an example. All miRNA backbone specific primer pairs are listed in the table below.

| Component | Volume (ul) | | | |
|---|---|---|---|---|
| NEBNext 2 × mix (M0541L) | 50 | | | |
| DMSO (D9170-5VL) | 2 | | | |
| Betaine (Sigma, B0300-1VL) | 10 | | | |
| 100uM FW primer (miR-1-1- | 0.5 | | | |
| AAV-H1-AMELY-V1-AarI- | | | | |
| FW) | | | | |
| 100uM RV primer (miR-1-1- | 0.5 | | | |
| AAV-H1-AMELY-V1-AarI- | | | | |
| RV) | | | | |
| 1ng EF1A oligo pool from | 37 | | | |
| Twist and nuclease free water | | | | |
| Total | 100 | | | |
| STEP | TEMP | | TIME | |
| Initial Denaturation | 98° | C. | 30 | s |
| 20 cycles | 98° | C. | 10 | s |
| | 64 | C | 30 | s |
| | 72° | C. | 15 | s |
| Final Extension | 72° | C. | 2 | minutes |
| Hold | 4-10° | C. | | |

Double sided bead purification with 0.7×SPRI beads and 1.2×SPRI beads ratios was used on the PCR product, which was in turn used as the insert in the HiFi assembly.

HiFi assembly of the pooled library was performed by assembling at 5 to 1 insert to backbone molar ratio. 15 ul of 2×HiFi assembly master mix (NEB, E2621L) and 15 ul of insert and backbone (about 0.375 pmol purified miR library insert to 0.075 pmol purified backbone) and incubating for 1 hr at 50° C.

Assembled DNA was precipitated by adding 1 ul of 20 mg/mL glycogen, one-tenth volume of 3M sodium acetate pH 5.5, and 2.2× volume of ethanol, mixed and stored overnight at −80° C.

Samples were spun at 16,000×g for 15 min at 4° C. Supernatant was removed and discarded. Pellets were washed twice with 1 ml of 70% ice cold ethanol and let to dry, then dissolved in 4 ul nuclease free water.

Purified DNA and 0.1 cm Gene Pulser Cuvettes (Bio-rad, 165-2089) were placed on ice for 10 min. 50 ul of Lucigen Endura electrocompetent cells were thawed briefly on ice. 4 ul of precipitated HiFi reaction was added to 25 ul of electrocompetent cells, mixed, and transferred to the cuvette. DNA and cell mixes were electroporated with the following parameters: 1800 Volts, 10 uF, 600 Ohms, 0.1 cm cuvette. Cuvettes were immediately flushed 2× with 1 mL Lucigen recovery media. Cells were recovered at 37° C. for 1 hour at 230 rpm.

To titer the transformed bacteria, 2 uL of each culture was diluted into 200 uL of LB and plated 100 ul and 10 ul of this 1:100 dilution onto LB agar plates plus appropriate antibiotic. The number of colonies were counted the next day to determine total number of transformants.

Liquid cultures were inoculated into the appropriate amount of LB with antibiotic for maxi prep. Pooled plasmid libraries were prepared with a Qiagen Plasmid Maxi Kit following the manufacturer's instructions.

### Primers

| Name | Sequence |
|---|---|
| miR-1-1- | Taagttctgtatgagaccaccatgcagactg |
| AAV-H1- | cctgctTGG |
| AMELY-V1- | (SEQ ID NO: 2227) |
| AarI-FW | |
| miR-100- | taagttctgtatgagaccacCCCAAAAGAGA |
| AAV-H1- | GAAGATATT |
| AMELY-V1- | (SEQ ID NO: 2228) |
| AarI-FW | |
| miR-124- | taagttctgtatgagaccacTTCCTTCCTCA |
| AAV-H1- | GGAGAAAGG |
| AMELY-V1- | (SEQ ID NO: 2229) |
| AarI-FW | |
| miR-128- | taagttctgtatgagaccacATTTtgcaata |
| AAV-H1- | attggcctt |
| AMELY-V1- | (SEQ ID NO: 2230) |
| AarI-FW | |
| miR-122- | taagttctgtatgagaccacggctacagagt |
| AAV-H1- | ttCCTTAGC |
| AMELY-V1- | (SEQ ID NO: 2231) |
| AarI-FW | |
| miR-130a- | taagttctgtatgagaccacgcagggccggc |
| AAV-H1- | atgcctcTG |
| AMELY-V1- | (SEQ ID NO: 2232) |
| AarI-FW | |
| miR-132- | taagttctgtatgagaccacGCCGTCCGCGC |
| AAV-H1- | GCCCCGCCC |
| AMELY-V1- | (SEQ ID NO: 2233) |
| AarI-FW | |
| miR-138- | taagttctgtatgagaccacgccggcggagt |
| 2-AAV-H1- | tctggtatC |
| AMELY-V1- | (SEQ ID NO: 2234) |
| AarI-FW | |
| miR-144- | taagttctgtatgagaccacTCAAGCCATGC |
| AAV-H1- | TTCCTGTGC |
| AMELY-V1- | (SEQ ID NO: 2235) |
| AarI-FW | |
| miR-155- | taagttctgtatgagaccacCCTGGAGGCTT |
| M-AAV-H1- | GCTGAAGGC |
| AMELY-V1- | (SEQ ID NO: 2236) |
| AarI-FW | |
| miR-155E- | taagttctgtatgagaccacCTGGAGGCTTG |
| AAV-H1- | CTTTGGGCT |
| AMELY-V1- | (SEQ ID NO: 2237) |
| AarI-FW | |
| miR-16-2- | taagttctgtatgagaccacTTATGTTTGGA |
| AAV-H1- | TGAACTGAC |
| AMELY-V1- | (SEQ ID NO: 2238) |
| AarI-FW | |
| miR-190a- | taagttctgtatgagaccacGAGCTCAGTCA |
| AAV-H1- | AACCTGGAT |
| AMELY-V1- | (SEQ ID NO: 2239) |
| AarI-FW | |
| miR-223- | taagttctgtatgagaccacTCCCCACAGAA |
| AAV-H1- | GCTCTTGGC |
| AMELY-V1- | (SEQ ID NO: 2240) |
| AarI-FW | |
| miR-451a- | taagttctgtatgagaccacGCTCTCTGCTC |
| AAV-H1- | AGCCTGTCA |
| AMELY-V1- | (SEQ ID NO: 2241) |
| AarI-FW | |
| miR-1-1- | TATGTGATATGCATAATAaaaaaaggccccc |
| AAV-H1- | gtggtgtggagtg |
| AMELY-V1- | (SEQ ID NO: 2242) |
| AarI-RV | |
| miR-100- | TATGTGATATGCATAATAaaaaaaGGCATAT |
| AAV-H1- | AAGCAAAGCCCCA |
| AMELY-V1- | (SEQ ID NO: 2243) |
| AarI-RV | |
| miR-124- | TATGTGATATGCATAATAaaaaaatcctTGG |
| AAV-H1- | CGGGCCCTCGCCG |
| AMELY-V1- | (SEQ ID NO: 2244) |
| AarI-RV | |
| miR-128- | TATGTGATATGCATAATAaaaaaaagcagtg |
| AAV-H1- | gaaacctgagtaa |
| AMELY-V1- | (SEQ ID NO: 2245) |
| AarI-RV | |
| miR-122- | TATGTGATATGCATAATAaaaaaacaaagca |
| AAV-H1- | aacgatgccaaga |
| AMELY-V1- | (SEQ ID NO: 2246) |
| AarI-RV | |
| miR-130a- | TATGTGATATGCATAATAaaaaaacaatgct |
| AAV-H1- | gaggaggcagcca |
| AMELY-V1- | (SEQ ID NO: 2247) |
| AarI-RV | |
| miR-132- | TATGTGATATGCATAATAaaaaaaGGCTCGG |
| AAV-H1- | GGCGCGGCGTGGC |
| AMELY-V1- | (SEQ ID NO: 2248) |
| AarI-RV | |
| miR-138- | TATGTGATATGCATAATAaaaaaaCCggtcc |
| 2-AAV-H1- | cacgaggctcgcc |
| AMELY-V1- | (SEQ ID NO: 2249) |
| AarI-RV | |
| miR-144- | TATGTGATATGCATAATAaaaaaatgtccTC |
| AAV-H1- | CTTGTCAGGCTCC |
| AMELY-V1- | (SEQ ID NO: 2250) |
| AarI-RV | |
| miR-155- | TATGTGATATGCATAATAaaaaaaCCGGTGG |
| M-AAV-H1- | CCATTTGTTCCAT |
| AMELY-V1- | (SEQ ID NO: 2251) |
| AarI-RV | |
| miR-155E- | TATGTGATATGCATAATAaaaaaaCCCACGG |
| AAV-H1- | TGGCCATTTGTTC |
| AMELY-V1- | (SEQ ID NO: 2252) |
| AarI-RV | |
| miR-16-2- | TATGTGATATGCATAATAaaaaaaAAACAAT |
| AAV-H1- | TGATAAAATAGTT |
| AMELY-V1- | (SEQ ID NO: 2253) |
| AarI-RV | |
| miR-190a- | TATGTGATATGCATAATAaaaaaaCTTTATT |
| AAV-H1- | AGGAACCCCCGGA |
| AMELY-V1- | (SEQ ID NO: 2254) |
| AarI-RV | |
| miR-223- | TATGTGATATGCATAATAaaaaaaGGCCTAG |
| AAV-H1- | AGCTGGTAAGCAT |
| AMELY-V1- | (SEQ ID NO: 2255) |
| AarI-RV | |
| miR-451a- | TATGTGATATGCATAATAaaaaaaCTGAGTT |
| AAV-H1- | CTCTTCCTGGCAC |
| AMELY-V1- | (SEQ ID NO: 2256) |
| AarI-RV | |

### Pooled AAV Production

AAV micropools served as cis-plasmids to package with Ad helper and AAV9 RepCap using standard three plasmid AAV packaging methods at Vector BioLabs.

### Crude Lysate Processing and Gel Visualization

To extract vector genomes, crude lysates underwent 4 freeze thaw cycles (37° C. and dry ice-ethanol bath) and were passed through a 0.45 um filter (Chemglass, CLS-2005-017). Each 100 ul of passthrough was treated with 2 ul DNAse 1 (NEB, M0303L) and 0.2 ul RNAse A (ThermoScientific, EN0531) for 30 min at 37° C. Vector genomes were extracted with the Quick Viral DNA kit (Zymo, D3015). 1.5% agarose gels with either SYBRsafe or SYBRgold to stain DNA were used for visualization.

### Pooled Expression of Micropools for Small RNAseq

Micropools of miR100 and miR100_M backbone miRs, embedded in the plasmid scAAV_AMELY_V1_H1, were transfected into HEK293 cells using a lipid based method (Lipofectamine LTX, ThermoFisher) in cells grown in 6 well plates. 600,000 cells were seeded per well and were transfected the following day in duplicate, with 2.5 micrograms of micropool library transfected per well. Media was changed at day 2 and collected in Trizol at day 3. Total RNA was extracted by chloroform phase separation and purification by Zymo Direct-zol column elution using manufacturer's protocols.

### Small RNAseq

Small RNAseq libraries were prepared using the Nextflex v3 small RNA seq kit (Bioo Scientific Corp, NOVA-5132-05). Briefly, library preparation was initiated with 0.5-2 ug of RNA input. 14-18 cycles of PCR were performed for each sample. Two rounds of double-sided bead cleanup were performed prior to pooling samples based on Tapestation High Sensitivity D1000 quantitation of the 150 bp band. Illumina adapted libraries were multiplexed and loaded onto a MiSeq (Illumina), loading the library at 9 pM with 10% phiX on a MiSeq v3 kit and with read 1 set to 75 cycles and index set to 6 cycles.

### Example 7: Ranking of Top Artificial miRNAs Embedded in miR-100 and miR-100 M Backbones

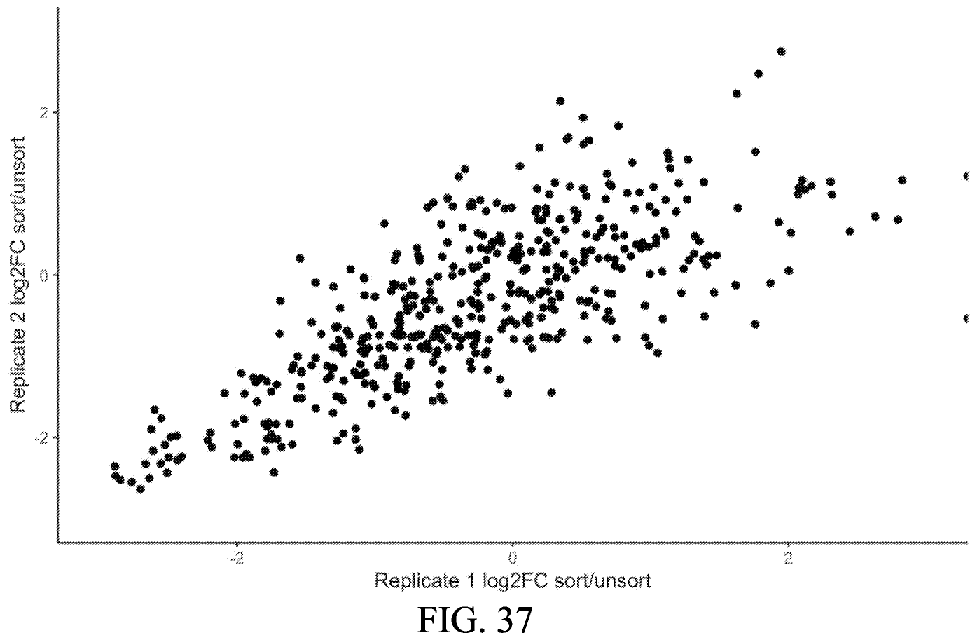

Top amiRNAs embedded in miR-100 and miR-100_M backbones were ranked by knockdown performance in Deep Screen 2; by guide to passenger ratio; and by minimal depletion at late (T₁, 18 day) versus early (T₀) timepoints (dropout). (Noting, as above, that the guide:passenger ratios are from a small RNAseq library including a mix of miR100 and miR100_M backbones). Additionally, the set of potential off-target transcripts with 1 or 2 bp mismatches was assessed for each ranked candidate. After eliminating candidates with low guide:passenger ratios, low T₁/T₀ ratios, and candidates with CNS expressed transcripts with near-complementarity of only 1 bp mismatch, a set of 9 active miRNAs, and 2 911 control miRNAs, were cloned into cis plasmids downstream of an H1 promoter, and packaged with a Rep/Cap helper plasmid encoding for AAV-DJ capsid components. Data from Deep Screen 2 (FIG. 37) and small RNAseq profiling for these candidates are listed in Table 25. For these selected hits, the mean of replicate 1 and replicate 2 T₁/T₀ log₂ ratios were all within 1 standard deviation (0.22) of the median (−0.07)log₂ ratio of miR100 and miR100_M amiRNAs targeting ATXN2.

TABLE 25
Data from Deep Screen 2 and small RNAseq
profiling

| | Rep. 1 | Rep. 2 | Rep. | Rep. | miR_ | Guide | Guide: |
|---|---|---|---|---|---|---|---|
| Po- | lo10/ | lo10/ | 1 | 2 | with_ | Se- | Pas- |
| si- | unsort | unsort | T₁/T₀ | T₁/T₀ | suf- | quences | senger |
| tion | log2FC | log2FC | log2FC | log2FC | fix | (DNA) | ratio |
| 1755 | −2.70 | −2.64 | 0.04 | −0.12 | miR- | TCGGGTT | 161 |
| | | | | | 100 | GAAATCT | |
| | | | | | | GAAGTGT | |
| | | | | | | G | |
| | | | | | | [SEQ ID | |
| | | | | | | NO: | |
| | | | | | | 657] | |
| 2586 | −2.40 | −2.24 | −0.28 | −0.08 | miR- | TAGATTC | 390 |
| | | | | | 100 | AGAAGTA | |
| | | | | | | GAACTTG | |
| | | | | | | G | |
| | | | | | | [SEQ ID | |
| | | | | | | NO: | |
| | | | | | | 1621] | |
| 2945 | −2.61 | −2.17 | 0.10 | −0.31 | miR- | TGTAGTA | >778:0 |
| | | | | | 100 | GAAGGCT | |
| | | | | | | TTGGCTG | |
| | | | | | | A | |
| | | | | | | [SEQ ID | |
| | | | | | | NO: | |
| | | | | | | 685] | |
| 3133 | −2.66 | −2.33 | 0.21 | 0.27 | miR- | TATGTCT | 78 |
| | | | | | 100_M | TGGCTTG | |
| | | | | | | ATTCACT | |
| | | | | | | G | |
| | | | | | | [SEQ ID | |
| | | | | | | NO: | |
| | | | | | | 1624] | |
| 3270 | −2.09 | −1.46 | 0.05 | 0.03 | miR- | TACTGTA | >334:0 |
| | | | | | 100 | GGCAACA | |
| | | | | | | TATTGCG | |
| | | | | | | T | |
| | | | | | | [SEQ ID | |
| | | | | | | NO: | |
| | | | | | | 2080] | |
| 3301 | −2.52 | −2.09 | −0.19 | −0.27 | miR- | TGAACAA | 92 |
| | | | | | 100_M | GGGGCTG | |
| | | | | | | ATTTGGG | |
| | | | | | | A | |
| | | | | | | [SEQ ID | |
| | | | | | | NO: | |
| | | | | | | 687] | |
| 3302 | −2.89 | −2.36 | −0.09 | −0.30 | miR- | TTGAACA | 41 |
| | | | | | 100_M | AGGGGCT | |
| | | | | | | GATTTGG | |
| | | | | | | G | |
| | | | | | | [SEQ ID | |
| | | | | | | NO: | |
| | | | | | | 688] | |
| 3330 | −2.18 | −2.12 | 0.30 | −0.06 | miR- | TATGCTG | 39 |
| | | | | | 100 | AGACTGA | |
| | | | | | | TAATGTG | |
| | | | | | | G | |
| | | | | | | [SEQ ID | |
| | | | | | | NO: | |
| | | | | | | 1614] | |
| 3338 | −1.91 | −2.25 | 0.03 | −0.15 | miR- | TACATGA | 212 |
| | | | | | 100 | GGATGCT | |
| | | | | | | GAGACTG | |
| | | | | | | A | |
| | | | | | | [SEQ ID | |
| | | | | | | NO: | |
| | | | | | | 1620] | |

The above miRNAs as well as 911 controls for 1755 (guide sequence SEQ ID NO:1185) and 2945 (guide sequence SEQ ID NO:1213) were tested for knockdown of ATXN2 in stem-cell derived motor neurons. amiRNAs were packaged in cis plasmids to generate self-complementary AAV-DJ vectors containing a long H1 promoter (nucleotides 113-343 of SEQ ID NO:2257), and a stuffer sequence “PSG11_V5” (nucleotides 489-2185 of SEQ ID NO:2257). Sequences for vectors encoding amiRNAs miR100_1755 (SEQ ID NO:1915), miR100_2586 (SEQ ID NO:1982), miR100_2945 (SEQ ID NO:1965), and miR100_3330 (SEQ ID NO:2021) from 5′ ITR to 3′ ITR are provided in SEQ ID NO:2257, SEQ ID NO:2258, SEQ ID NO:2259, and SEQ ID NO:2260, respectively. After titering each vector, and based on hemacytometer based quantification of number of cells plated, vectors were added at intended doses of 3.16E3 and 3.16E4 vector genomes per cell. 7 days after addition of vectors, neurons were harvested and RNA isolated with miRNeasy Tissue/Cells Advanced Mini Kit (Qiagen, P/N 217604) ATXN2 knockdown was assessed by digital droplet RT-PCR, measuring the ratio of ATXN2 expression to housekeeping controls GUSB and B2M.

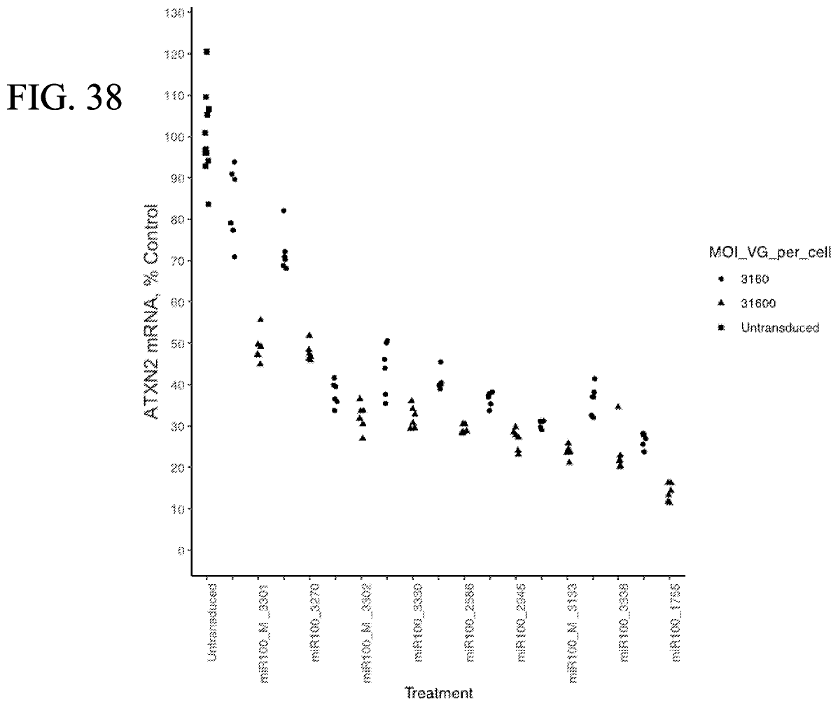

FIG. 38 shows individual data points, and Table 26 shows mean and standard deviation of knockdown across these constructs, at the two doses of 3.16E3 vg/cell and 3.16E4 vg/cell, normalized to ATXN2 expression values from untransduced cells, which were treated with an equivalent volume of AAV diluent.

TABLE 26
ATXN2 Knockdown by amiRNAs in stem-cell derived
motor neurons at two different doses

| miR | mean_ATXN2_3160 | SD | N | mean_ATXN2_31600 | SD | N |
|---|---|---|---|---|---|---|
| miR100_1755 | 26.8 | 1.7 | 6 | 14.0 | 2.1 | 6 |
| miR100_2586 | 41.8 | 2.9 | 6 | 29.3 | 1.0 | 6 |
| miR100_2945 | 36.7 | 1.7 | 6 | 26.8 | 2.6 | 6 |
| miR100_3270 | 72.1 | 5.1 | 6 | 47.9 | 2.2 | 6 |
| miR100_3330 | 44.1 | 6.3 | 6 | 32.2 | 2.7 | 6 |
| miR100_3338 | 36.5 | 3.6 | 6 | 23.6 | 5.5 | 6 |
| miR100_M_3133 | 30.6 | 1.0 | 5 | 23.8 | 1.5 | 6 |
| miR100_M_3301 | 83.7 | 9.1 | 6 | 49.1 | 3.7 | 6 |
| miR100_M_3302 | 38.0 | 3.0 | 6 | 32.3 | 3.3 | 6 |
| Untransduced | 100 | 9.5 | 12 | | | |

### Dose Response Studies

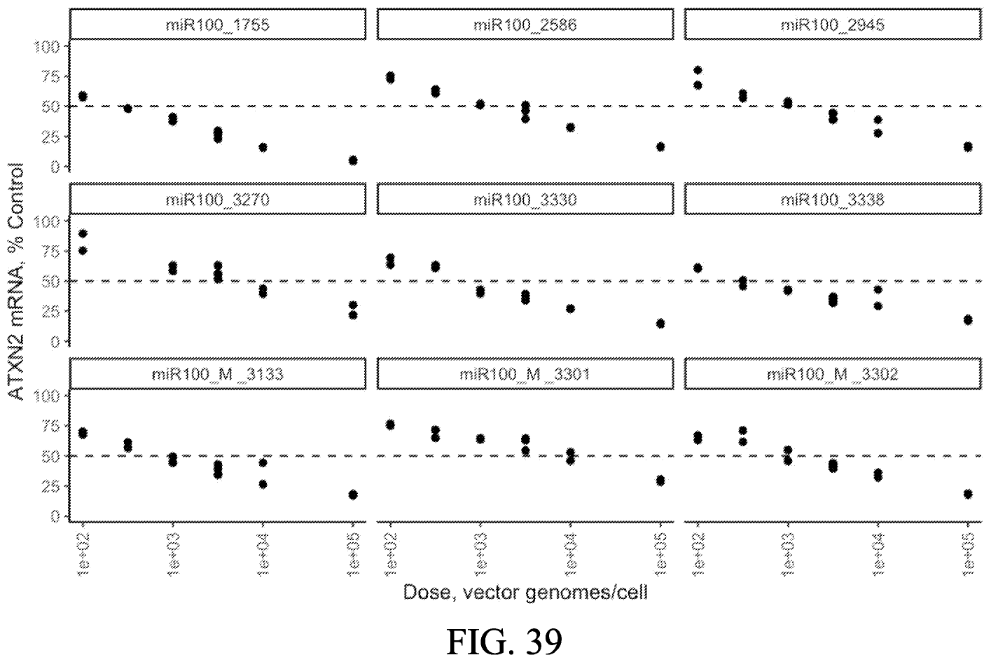

The candidates AAVs were also tested at a more extensive range of doses in motor neurons. As before, RNA was isolated from the cultures after 7 days of culture, and ATXN2 knockdown assessed. FIG. 39 shows plots of knockdown across different concentrations of each vector added. Concentration of ATXN2 mRNA, normalized for each data point by B2M expression, and collectively to the ATXN2 expression level in neurons treated with vehicle (PBS+0.001% PF-68) was measured by digital droplet RT-ddPCR. By examination, differences in potencies of amiRNAs can be observed; for example miR100_1755 exhibits knockdown at lower vector genome exposures than other amiRs; mir100_3301 and miR100_3270 appear to exhibit reduced potency relative to other vectors.

Neurons dosed at 3.16E3 vector genomes per cell were additionally subject to small RNA sequencing. Table 27 shows the abundance of the amiRNA, as a fraction of total miRNA. There was a surprising range of expression levels, and several amiRs (1755, 2586, 2945, and 3270) had considerably less amiRNA detected than other amiRNAs.

For these small RNA experiments, reads were not ‘deduplicated’ (by eliminating reads with identical flanking 5′ and 3′ 4-mer random adapters) as in small RNA analysis for deep screen 1 libraries, because the number of reads of the artificial miRNAs in some cases approached the number of unique combinations of nucleotides in the adapters.

TABLE 27
Abundance of amiRNA, as a fraction of total miRNA

| | Rep1 amiRNA/ | Rep2 amiRNA/ |
|---|---|---|
| Guide | total miRNA (%) | total miRNA (%) |
| mir100_1755 | 1.80 | 2.77 |
| mir100_2586 | 2.53 | 2.73 |
| mir100_2945 | 3.20 | 3.30 |
| mir100_3270 | 1.21 | 1.43 |
| mir100_3330 | 16.83 | 17.75 |
| mir100_3338 | 23.84 | 25.00 |
| mir100M_3133 | 22.82 | 22.57 |
| mir100M_3301 | 22.07 | 17.53 |
| mir100M_3302 | 38.04 | 36.78 |

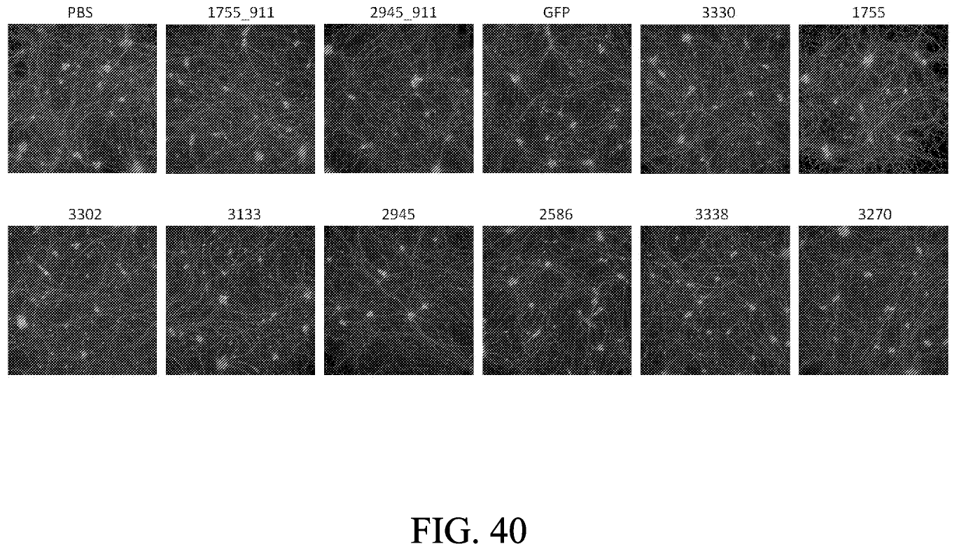

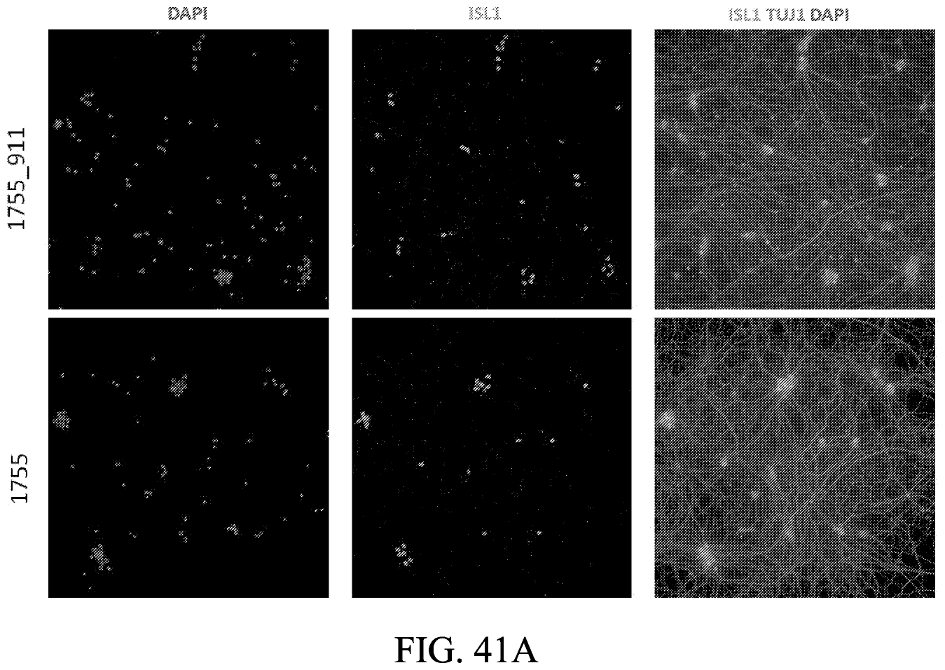

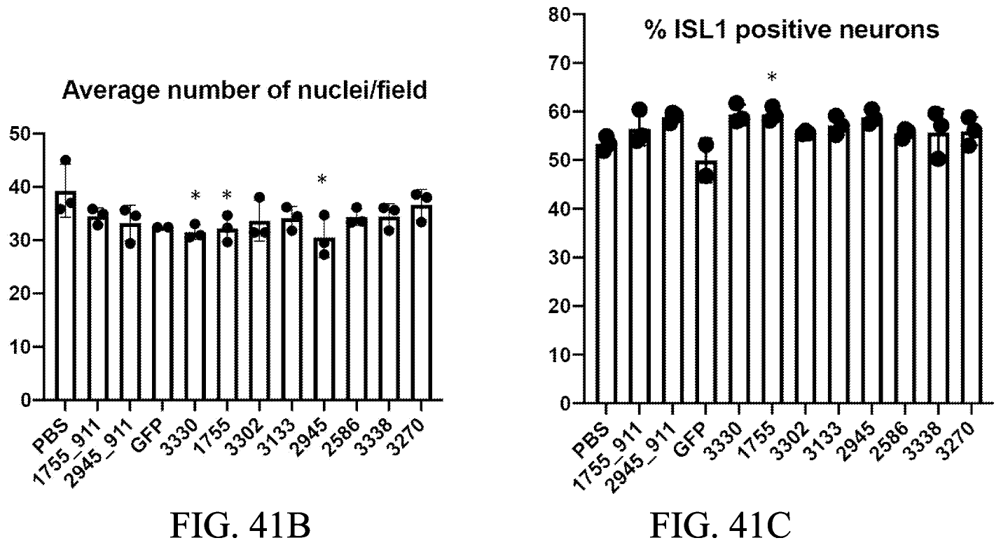

To assess whether AAV amiRNA treatment had any obvious impact on neuronal morphology or cell counts, neurons grown in 96-well format were treated with AAV or vehicle at a dose of 1E4 vector genomes/cell, and 7 days later fixed and stained with Hoechst, anti-Isl1, and anti-Beta3 tubulin antibodies to visualize nuclei, a motor neuron marker, and neuronal processes respectively. FIG. 40 shows representative images from cultures treated with indicated amiRNA AAVs and controls, demonstrating that no AAV miRNA exhibited obvious impacts on neuronal morphology. FIG. 41A shows zoomed in images comparing miR100_1755 and miR100_1755_911 (a 911 control, rendered inactive for slicing Atxn2 by complementing bases 9, 10 and 11 of the 1755 amiRNA). No obvious differences can be seen, suggesting that Atxn2 knockdown does not cause dramatic changes in neuronal process or nuclear morphology. Panels on right quantify the total number of Hoechst+ nuclei (FIG. 41B) and the % of total nuclei that are Isl1+ (FIG. 41C). Compared to vehicle-treated (PBS+0.001% PF-68) wells, significant differences (p<0.05) were observed for a few of the AAV-amiRNA treatments, with a reduction in total number of nuclei per field. However, one of these treatments (miR100_1755) were also showed with a significant increase in the fraction of cells that were Isl1+, and an apparent trend toward increasing Isl+ neurons was apparent for other AAV-DJ amiRNAs, arguing against any alteration in total motor neuron numbers. There were no significant differences between neurons transduced with any of the active AAV amiRNAs and the inactive 911 control AAV amiRNAs.

### RNAseq Studies

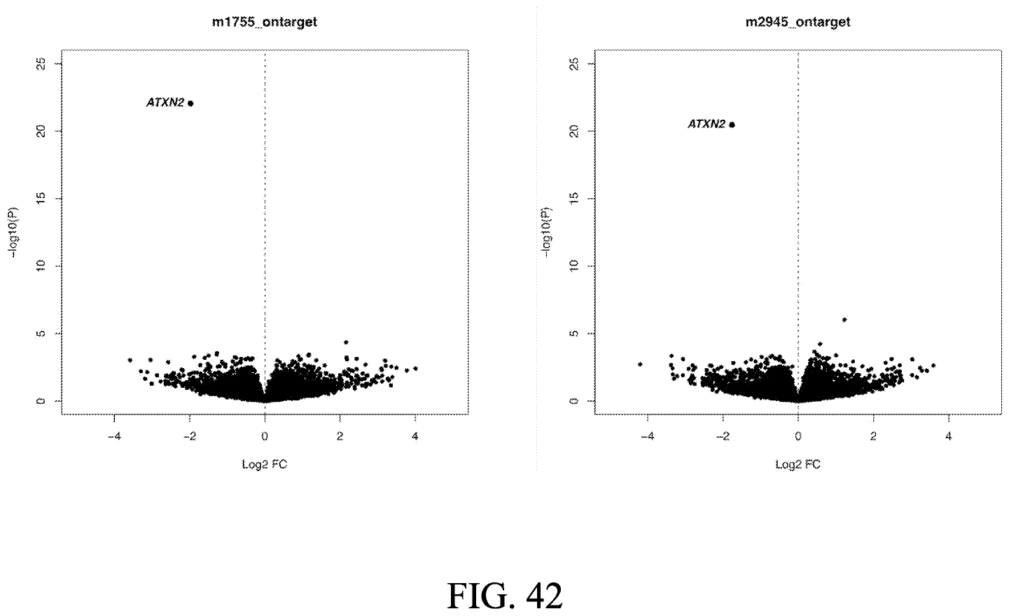

RNA was collected from motor neurons 7 days after dosing with 1E4 vector genomes/cell of the above AAVs. There were 6 replicates per condition, except miR100_1755_911, which had 5. To determine if ATXN2 knockdown from AAV expression impacts the transcriptome in neurons, RNA expression was compared between neurons transduced with active amiRNA-expressing vectors and vectors expressing a cognate 911 control. FIG. 42 shows ‘volcano plots’ of differential expression for miR100_1755 vs. miR100_1755_911 and miR100_2945 and miR100_2945_911. A large separation can be seen in nominal p-values for the differential expression calculated for ATXN2 versus all other genes. Remarkably, after adjustment of nominal p values for multiple comparisons using the Benjamini-Hochberg procedure, only ATXN2 or one other gene exceeded a 10% false discovery rate threshold for 1755 and 2945, respectively.

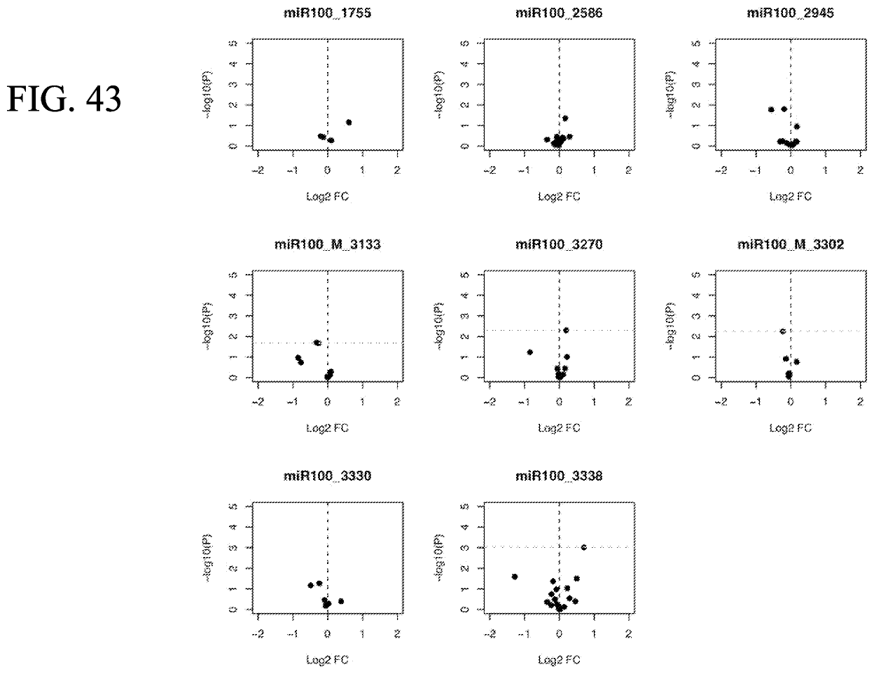

To further investigate whether there was any impact on any of the predicted off-target genes (the set of transcripts with 2 or fewer mismatches to bases 2-18 of each amiRNA), each amiRNA was compared to data from all other active amiRNAs (FIG. 43). For this set of selected amiRNAs, few of the predicted off-targets exceed the 10% false discovery rate threshold. This suggests that these amiRNAs yield specific knockdown of ATXN2.

### Methods

ddPCR AAV Titering

To titer AAVs, each vector was serially diluted in Salmon Sperm DNA solution (20 ng/ul Salmon Sperm DNA, 0.001% PF-68, 10 mM Tris-HCl pH 7.5, 50 mM KCl, 1.5 mM MgCl₂) and subsequently heated at 95° C. for 10 minutes to release the vector genome from the AAV9 capsid. After an incubation with SmaI to reduce secondary structure, known to inhibit the rAAV PCR reactions, (NEB, R0141L), droplets were generated using DG32 Automated Droplet Generator (Bio-Rad), followed by a PCR amplification with vector-specific primer/probe sets. Once complete, droplets were analyzed using QX200 Droplet Digital PCR System (Bio-Rad), and positive and negative populations were definded, and the dilution factor applied to determine the concentration of the undiluted vector stock.

### Motor Neuron Immunocytochemistry

Motor neuron cultures were fixed in 4% Paraformaldehyde for 10 minutes at room temperature. Fixed cultures were permeabilized and blocked in PBS containing 0.2% Triton-X-100 and 10% donkey serum solution for 45 minutes at room temperature. Cells were then incubated in blocking solution (10% donkey serum in 0.1% Tween-PBS) containing primary antibody overnight at 4 C. Cells were washed 3 times with PBS-0.1% Tween and then incubated in blocking solution containing secondary antibodies for 1-2 hours at room temperature followed by 3 washes with PBS-0.1% Tween and a rinse with a PBS solution containing Dapi. Stained cultures were imaged on the Perkin Elmer Operetta high content imager with 20× water objective. 40-60 fields were imaged for every well. Cell quantifications were done using the Perkin Elmer Harmony software. Statistical analysis was done using GraphPad Prism software. Primary antibodies used: TUJ1 (1:500 dilution) ISL1 (1:200 dilution) secondary antibodies: AlexaFluor 488 and AlexFluor 647 (1:500 dilutions).

| Reagent | Vendor | Cat. No. |
|---|---|---|
| 32% Paraformaldehyde | Fisher Scientific | 50-980-495 |
| Triton X-100 | Sigma | T8787-100ML |
| Tween 20 | Sigma | P1379-100ML |
| Donkey Serum | Jackson Immuno | 017-000-121 |
| | Research | |
| Donkey anti-chicken secondary antibody- | Jackson Immuno | 703-606-155 |
| Alexa fluor 647 | Research | |
| ISL1 antibody | Abcam | ab 109517 |
| TUJ1 antibody | Abcam | ab41489 |
| Donkey anti-rabbit secondary antibody- | ThermoFisher | A32790 |
| Alexa fluor 488 | | |
| DAPI solution | ThermoFisher | 62248 |
| Phosphate-Buffered Saline (PBS) pH 7.4 | ThermoFisher | 10010023 |

### Off-Target Prediction

To generate a set of predicted off-targets, bases 2-18 of amiRNAs were aligned to the human transcriptome using bowtie commands:

bowtie -n 2 -l 17 -e 81 -seed [pseudorandom number to enforce reproducibility]-nomaqround -tryhard -chunkmbs 256 --all --time (and additional commands for input/output handling). To ensure that only 2 or fewer mismatches occurred, fastq file inputs to the bowtie alignment containing amiRNAs to be tested were constructed in which each amiRNA was given a phred score ‘mask’ of IIIIIIIIIIIIIIIII, such that alignments of the amiRNA with transcripts where more than 2 mismatches occurred would exceed the weight threshold. The amiRNas were aligned to the build Homo_sapiens.GRCh38.cdna.all, Macaca_fascicularis.Macaca_fascicularis_5.0.cdna.all, or Mus_musculus.GRCm38.cdna.all.

### RNAseq

Stem-cell derived motor neuron cultures were plated at a density of 200,000 cells per well of 6-well plates. 6 days after plating, cells were transduced with AAV vectors at a dose of 10,000 vector genomes (calculated by titering method described above) per cell. 7 days later, cells were harvested for RNA._Lysis of transduced samples was conducted by addition of 300 ul of Buffer RLT Plus, followed by overnight freeze at −80. Samples were thawed on ice and processed according to the remainder of the RNeasy Plus standard protocol. (Qiagen RNeasy Plus Micro Kit (Catalog 74034)), according to manufacturer's instructions. All purified RNA samples were quantified by Qubit (using RNA HS standard). A selection of samples with low, mid, and high RNA concentrations (16 in total) were further characterized by Tapestation (High Sensitivity RNA) to check purity (RINe score) and verify Qubit quantification. All RINe scores were in the 9.9-10 range, near maximal.

Purified RNAs were then used as input into QuantSeq [Lexogen catalog #015 (QuantSeq 3′ mRNA-Seq Library Prep Kit for Illumina (FWD)]. Target RNA input was 100 ng per reaction (for lower concentration samples, the maximum input volume of 5 ul was used). The standard Quantseq protocol was followed with the following modifications: (1) UMI addition at step 7 using the “UMI Second Strand Synthesis Module” (Lexogen Cat. No. 081). (2) 20 cycles for library amplification. Resulting libraries were quantified by Qubit (DNA HS) and QC spot-checked on Tapestation (HS D5000). Libraries were pooled based on Qubit quantifications and sequenced on an Illumin NovaSeq (Seqmatic). Sequencing parameters were as follows: NovaSeq S1 run, single-read 100 bp, single index 6 bp.

### RNAseq Analysis

To analyze RNAseq data, SeqTK was used to split each of the single-end reads obtained from each sample into fastq files containing the UMI and read sequence, respectively:

seqtk trimfq -b 10 raw.fastq > sequence.fastq
seqtk trimfq -e [readsize - 6] raw.fastq > umi.fastq

The resulting sequences were then pseudoaligned with kallsito version 0.46.0 (Bray et al., Nature Biotechnology 2016 34: 525-527). in batch mode to a transcriptome assembly derived from the the trailing 600 bp of all cdnas present in Ensembl release 96 (kmer length=19) using the following command:

kallisto pseudo --umi --quant --single -t 8 -i [kallisto_index] -o
[output_file] - b [batch_file.txt]

Aligning reads were summed across all transcripts annotated to each gene to generate gene-level count matrices. Genes with five or more counts observed in all replicates of at least one experimental condition were considered in downstream analyses. Sample read counts were converted to base-2-log(CPM) and normalized via TMM (edgeR::calcNormFactors) prior to probability weight estimation via limma::voom. (Law et al., Genome Biology (2014) 15:R29). Evidence for differential expression was quantified by fitting a genewise linear model on the normalized expression values, with fold changes extracted from the model coefficients and associated P-values estimated using a Wald test. Genewise P-values were corrected for multiple testing using the FDR approach.

### Example 8: In Vivo Testing of Candidate amiRNAs in Wild-Type Mouse

Two additional studies of in vivo performance of amiRNAs embedded in self-complementary AAV9 vectors were conducted. In a first study, amiRNA 1784 and 3330, in the miR1-1 or miR100 backbone, respectively, were tested in a variety of vector genomes containing different promoters and stuffers. The specific miR cassettes used for in vivo testing are provided in Table 28.

TABLE 28
Specific miR cassettes used in vivo

| Cassette | Sequence |
|---|---|
| miR1- | CATGCAGACTGCCTGCTTGGGTACAGACCAAAGAGTA |
| 1.1784 | GTCGAATTATGGACCTGCTAAGCTAATTAACTACTCTT |
| | TGGTCTGAACTCAGGCCGGGACCTCTCTCGCCGCACTG |
| | AGGGGCACTCCACACCACGGGGGCC |
| | (SEQ ID NO: 538) |
| miR | CCCAAAAGAGAGAAGATATTGAGGCCTGTTGCCACAT |
| 100.3330 | ATGCTGAGACTGATAATGTGGGTATTAGTCCGCCACAT |
| | CATCCGTCTCAACATTTGTGTCTGTTAGGCAATCTCAC |
| | GGACCTGGGGCTTTGCTTATATGCC |
| | (SEQ ID NO: 2019) |

The vector designs, including specific promoter and stuffer, are described separately. Here the performance of the amiRNAs is compared in several overall vector formats and promoters. AAV was dosed to wild-type mice either intravenously (dose: 3.21E9 vg/gram mouse) or by intrastriatal injection (dose: 7.5E9 vg total).

Table 29 shows mean ATXN2 knockdown as assessed in liver 3 weeks after intravenous dosing, relative to animals dosed with vehicle (PBS with 0.001% PF-68). Atxn2 expression was assessed by digital droplet RT-PCR, and knockdown was taken as the mean of Atxn2/Hprt and Atxn2/Gusb ratios, as measured by ddPCR.

TABLE 29
ATXN2 Knock-down in Liver After I.V. amiRNA Dosing

| | mean Atxn2 | | |
|---|---|---|---|
| miR | (% Vehicle injected) | SD | N |
| miR1-1.1784 | 10.8 | 6.5 | 12 |
| miR100.3330 | 14.7 | 4.7 | 12 |

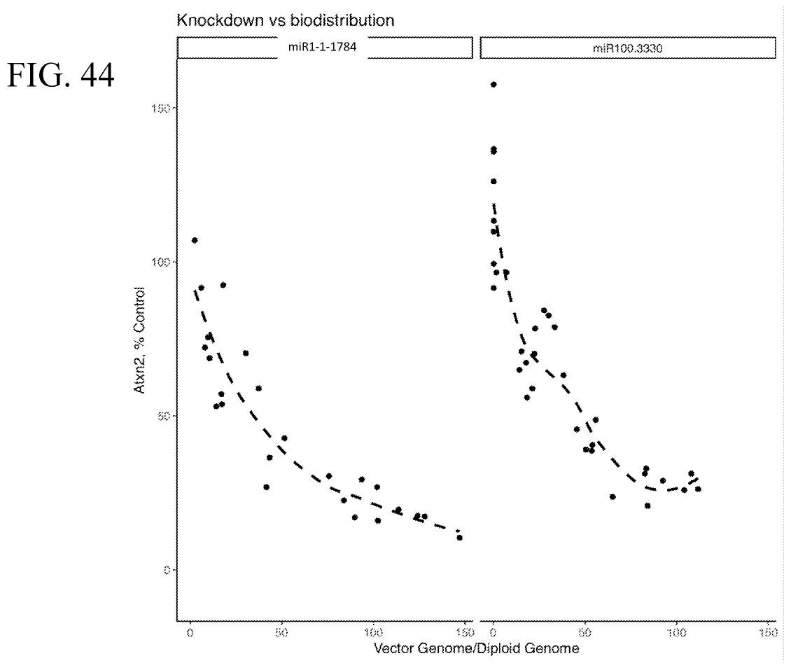

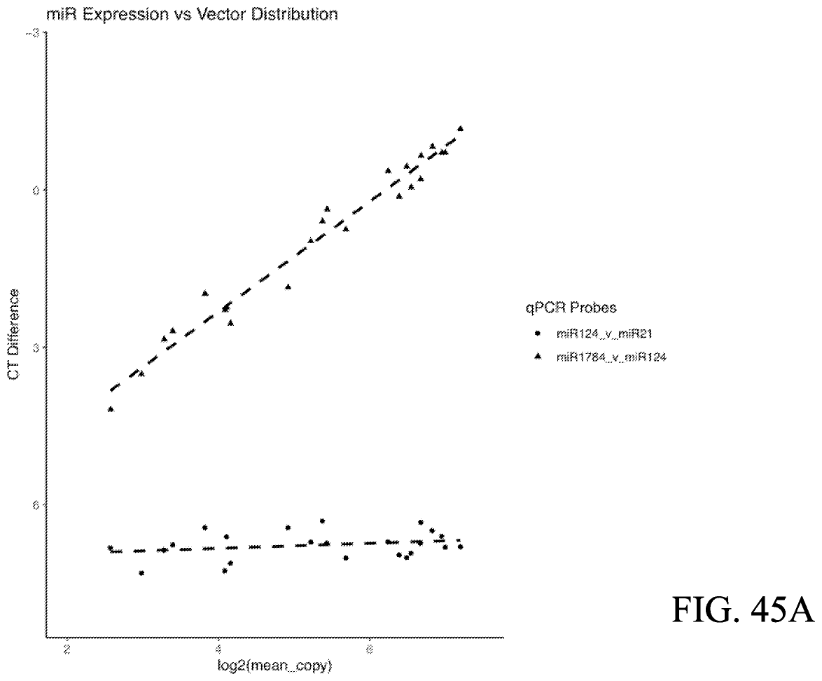

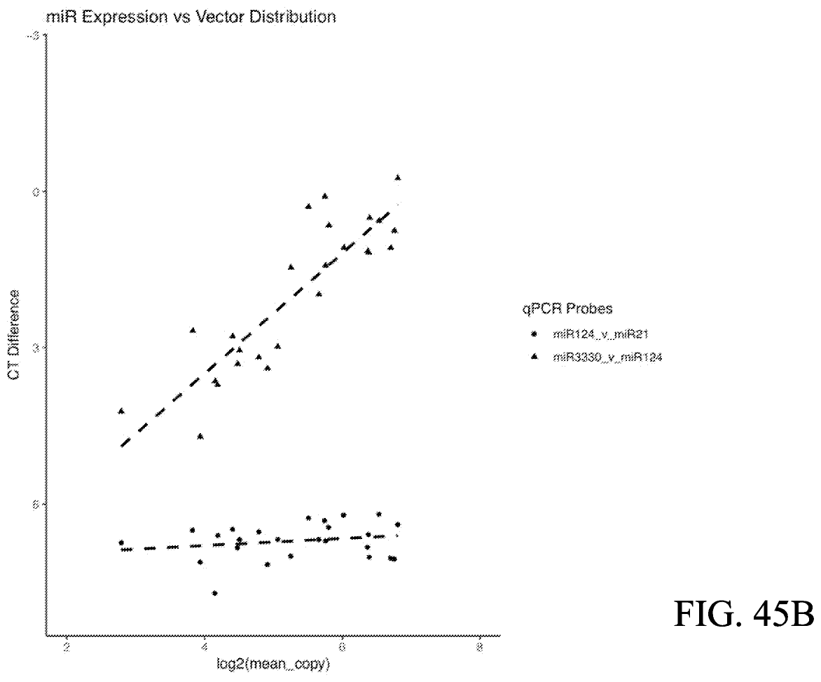

For striatal samples, vector biodistribution after collection of punch biopsies was more variable from sample to sample. Vector distribution was assessed by digital droplet PCR, measuring the relevant number of droplets amplifying for primer/probesets recognizing the AAV vector genome versus primer/probesets recognizing the Tert gene in the mouse genome. Because there are a fixed number of copies of the Tert gene per cell (2), the number of vector genomes per cell (diploid genome) can be measured in this way. By assessing AAV vector distribution in the same biopsies as ATXN2 mRNA was quantified, a clear dose response trend can be seen (FIGS. 44, 45A-45B). It should be noted that the amount of nuclear vector genomes versus cytoplasmic or extracellular vector was not assessed, such as by histological methods; it is possible that vector introduced by intraparenchymal injections may accumulate extracellularly. Nonetheless, the clear dose response shows that even if not all of the vector genomes measured are in the nucleus, available to express the amiRNA, there is a clear correlation between any such total vector genome exposure and functionally active vector genomes.

To determine the relationship between amiRNA expressed and knockdown, amiRNA was quantified in two ways. First, libraries using TaqMan Advanced miRNA cDNA Synthesis Kit (Thermo, P/N A28007) were generated for all striatal punch biopsy samples, using RNA isolated with a kit which enriches for small RNAs (Qiagen, P/N 217604). To generate a cDNA library for TaqMan qPCR, 3′ poly-A tailing is first complete, then 5′ ligation to add on an adaptor. After reverse transcription, the cDNA is PCR amplified for 14 cycles, then a dilution of the final amplification product is subject to qPCR with primer probe sets specific to exogenous and endogenous miRNAs. Primer/probesets designed to target exogenous amiRNAs were used (Thermo), as well as primer/probesets targeting endogenous miRNAs miR-21a-5p (Thermo, P/N mmu482709_mir) and miR-124-3p (Thermo, P/N mmu480901_mir) as controls. The abundance of miRNA is assessed by the qPCR cycle number at which target amplification occurs. Comparing the qPCR cycle where amplification occurs (CT) between primer/probesets targeting different miRNAs allows assessing the relative abundance of miRNAs.

FIGS. 45A-45B plots the difference in CT value between amiRNA and endogenous control, as well as the difference between two endogenous miRNAs (miR-21 and miR-124), against the vector biodistribution in the same sample. As can be seen, there is no obvious change in the difference in CT thresholds between endogenous miRNAs with increasing detection of AAV vector genome. By contrast, there is what appears to be a log-linear relationship between the expected increase in the CT separation between the amiRNA and endogenous miRNA and vector exposure, consistent with greater amiRNA expression with increased exposure to AAV.

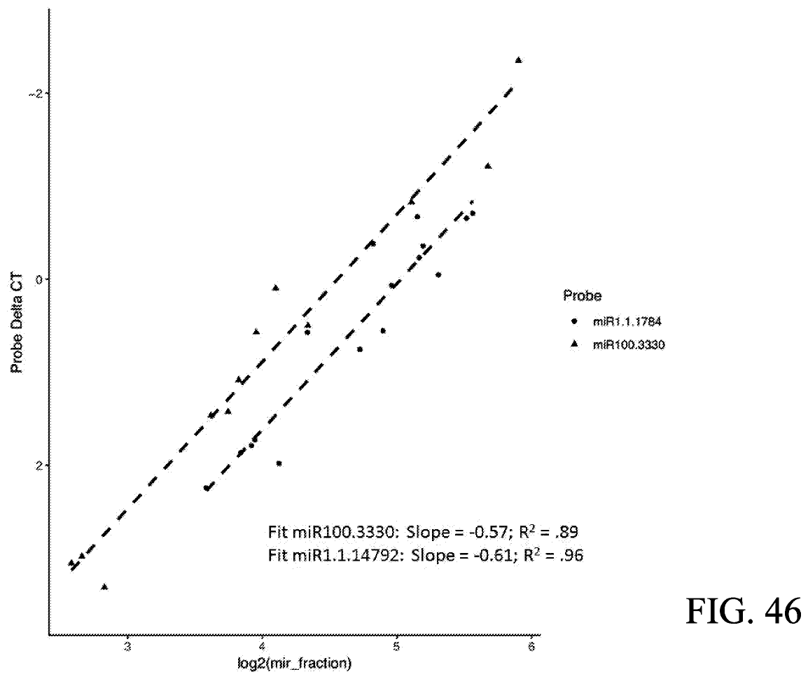

For a subset of samples, small RNAseq was additionally conducted. As above, amiRNA expression normalized by total miRNA expression was quantified for each sample. Since for these samples amiRNA expression was quantified both by small RNAseq and qPCR, a model could be fit to establish how qPCR predicts amiRNA expression as a function of total miRNA. Therefore a linear model was fit (FIG. 46), with good explanation of the variance (R²>0.89) for both amiRNAs.

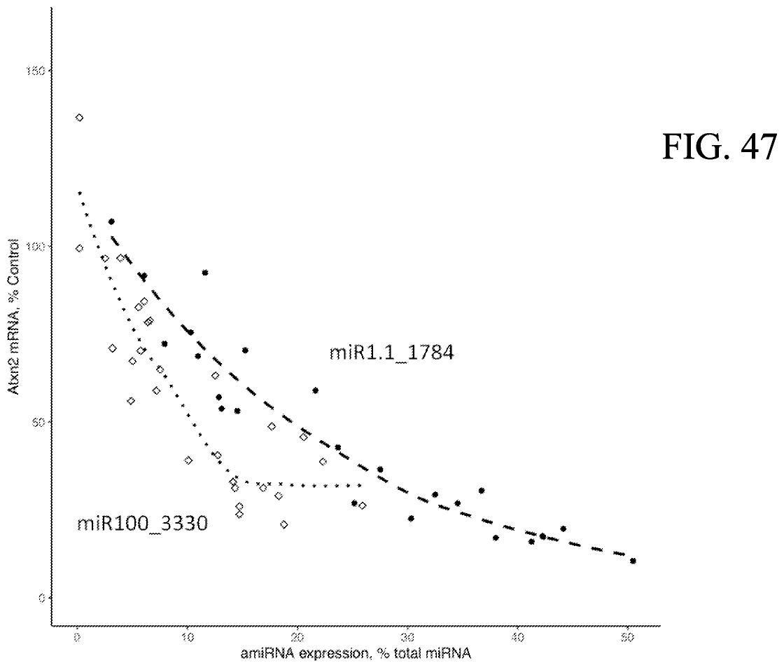

Using this model, the qPCR-assessed amiRNA expression values for miR100_3330 and miR1.1.1784 in all samples could be converted to an absolute scale, of amiRNA/total miRNA. Plotting ATXN2 mRNA in striatal biopsies versus this metric of predicted amiRNA expression, there was considerably greater knockdown per miRNA expressed in samples expressing the miR100-3330 amiRNA versus samples expressing the miR1.1.1784 amiRNA (FIG. 47). Therefore, although as a function of vector dosed, more knockdown was induced by vectors expressing miR1.1.1784, as a function of amiRNA expressed, more knockdown was induced by miR100.3330. This suggests that in vivo the potency of the approximately 22 nucleotide final product of pri-miRNA processing is higher for miR100.3330 than for miR1.1.1784.

In a second study, self-complementary vectors expressing amiRNAs miR100_1755 (SEQ ID NO:1915), miR100_2945 (SEQ ID NO:1965), miR100_3330 (SEQ ID NO:2021), and miR100_2586 (SEQ ID NO:1982) were packaged in AAV9 with a cis plasmid as described above containing a stuffer sequence “PSG11_V5” (nucleotides 489-2185 of SEQ ID NO:2257), a long H1 promoter (nucleotides 113-343 of SEQ ID NO:2257) and dosed intravenously or intrastriatally in adult wild-type mice. 5′ ITR to 3′ ITR sequences for these vectors, as described in Example 7, are provided in SEQ ID NO:2257 (scAAV_H1_long_miR100_1755_PSG11_V5_ITR_to_ITR.gb), SEQ ID NO:2258 (scAAV_H1_long_miR100_2586_PSG11_V5_ITR_to_ITR.gb), SEQ ID NO:2259 (scAAV_H1_long_miR100_2945_PSG11_V5_ITR_to_ITR.gb), and SEQ ID NO:2260 (scAAV_H1_long_miR100_3330_PSG11_V5_ITR_to_ITR.gb). Because the mouse Atxn2 transcript has several mismatches to 2586, no knockdown of mouse Atxn2 transcript is expected.

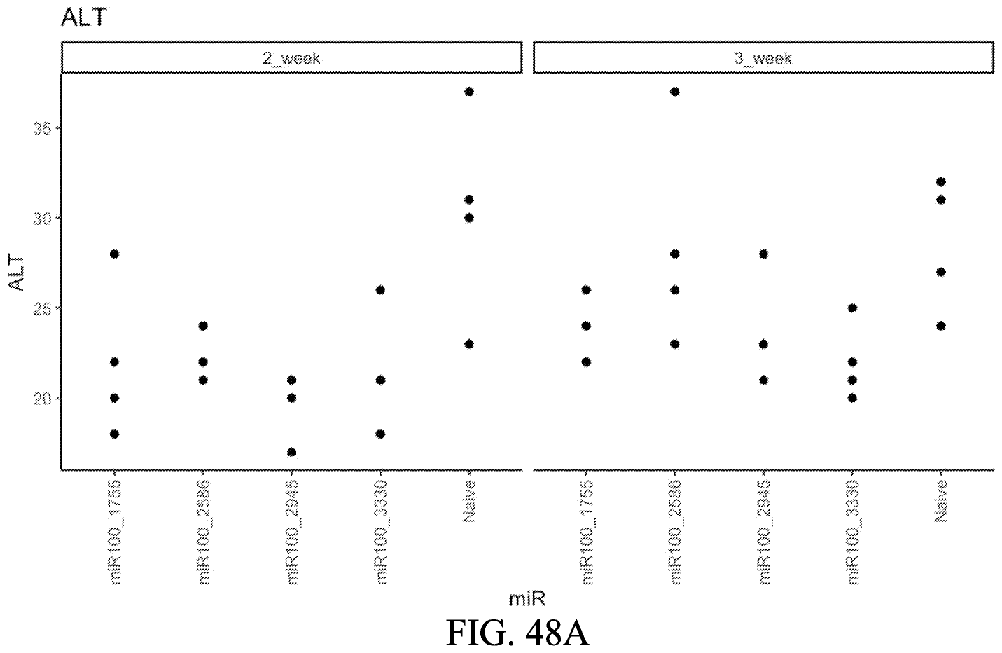

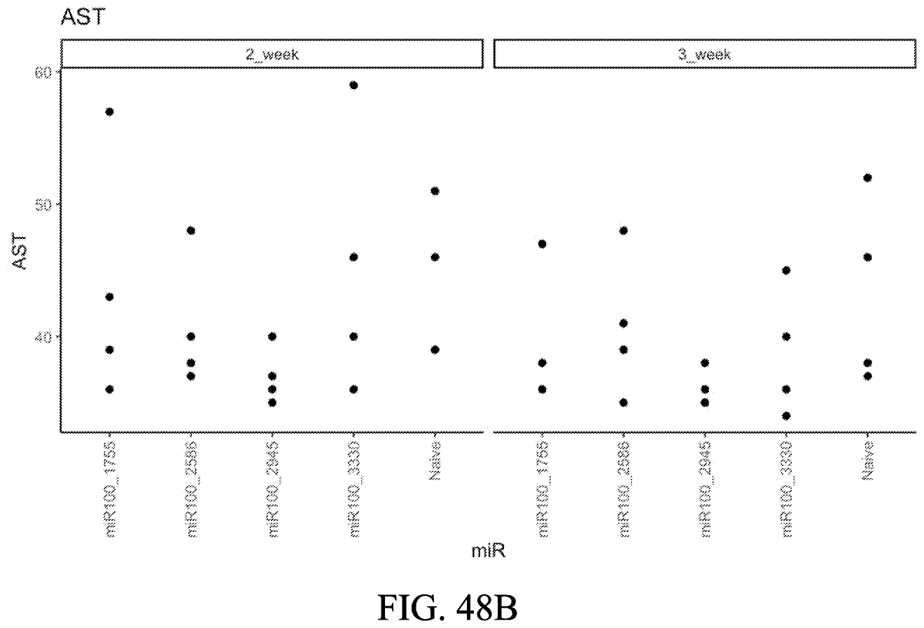

During the intravenous study, 4 animals were dosed per group for a 3-week study. There were no clinical observations noted during weekly observation. For ALT and AST analysis, blood was collected via submandibular vein into serum tubes and allowed to clot for 30 minutes. Samples were centrifuged at 12,000 rpm for 5 minutes at 4° C. Serum was collected into clean Eppendorf tubes and stored at −80° C. until further analysis at IDEXX. Results were reported as AST (U/L) and ALT (U/L). FIG. 48 shows liver enzyme data at 2 and 3 weeks post-dosing. All ALT and AST values were within normal ranges at these timepoints.

During the intrastriatal study, 6 animals were dosed 4 microliters per striatum per group for a 3-week study. There were no group wide clinical observations noted for 7 days following injection and during weekly observation and there were no unscheduled deaths. For one cage dosed with miR100-2586, fighting was observed but the bully was separated, and all animals completed the study.

Knockdown performance of vectors was tested in liver. Table 30 quantifies remaining Atxn2, normalizing Atxn2 to two different control genes (Hprt and Gusb) and further normalized to Atxn2 expression levels in naïve animals. From the same liver samples, as above biodistribution was measured. Samples treated with different vectors had highly similar exposures in liver.

TABLE 30
Atxn2 knockdown of amiRNA containing vectors in liver

| | Mean % | | | | |
|---|---|---|---|---|---|
| | Control | | Mean | | |
| | Atxn2 | mRNA | Biodistribution | SD | |
| Treatment | mRNA | SD | (VG/DG) | (VG/DG) | N |
| miR100_1755 | 22.1 | 3.3 | 5.93 | 1.70 | 4 |
| miR100_2945 | 29.3 | 5.0 | 5.77 | 0.50 | 4 |
| miR100_3330 | 27.9 | 0.8 | 5.71 | 0.36 | 4 |
| None | 100.0 | 10.5 | 0.01 | 0.01 | 4 |

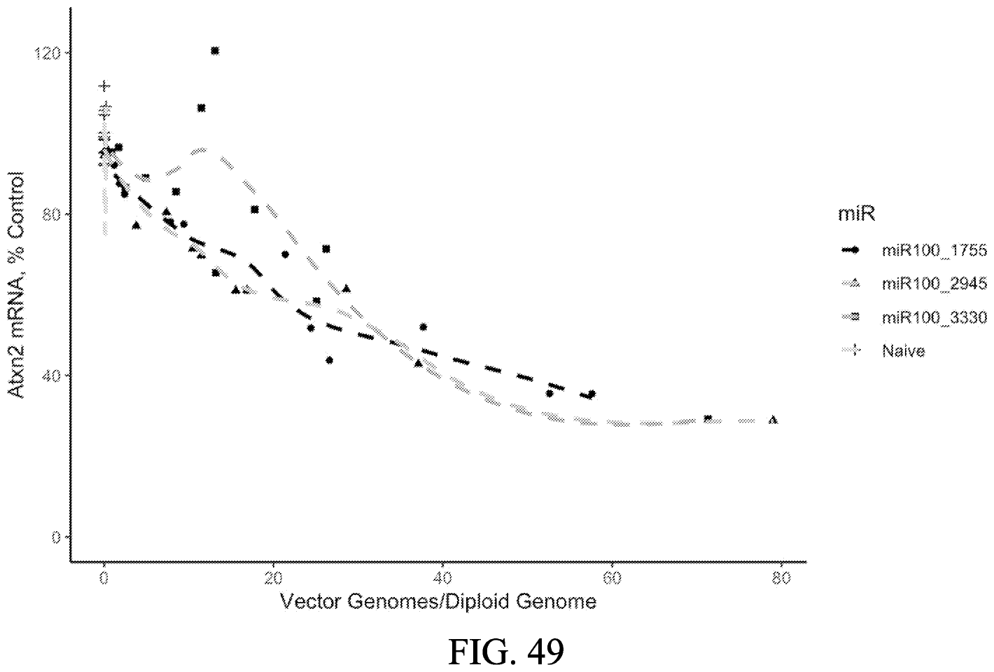

Knockdown performance of these vectors was further assessed in brain after intrastriatal injections. As in the study described above DNA, mRNA and small RNA were isolated from punch biopsies in order to simultaneously monitor vector biodistribution, Atxn2 knockdown, and amiRNA expression. Although in this in vivo study exposure levels were lower than in the above study with miR1.1.1784 and miR100_3330, for unknown reasons, a clear dose response is visible (FIG. 49).

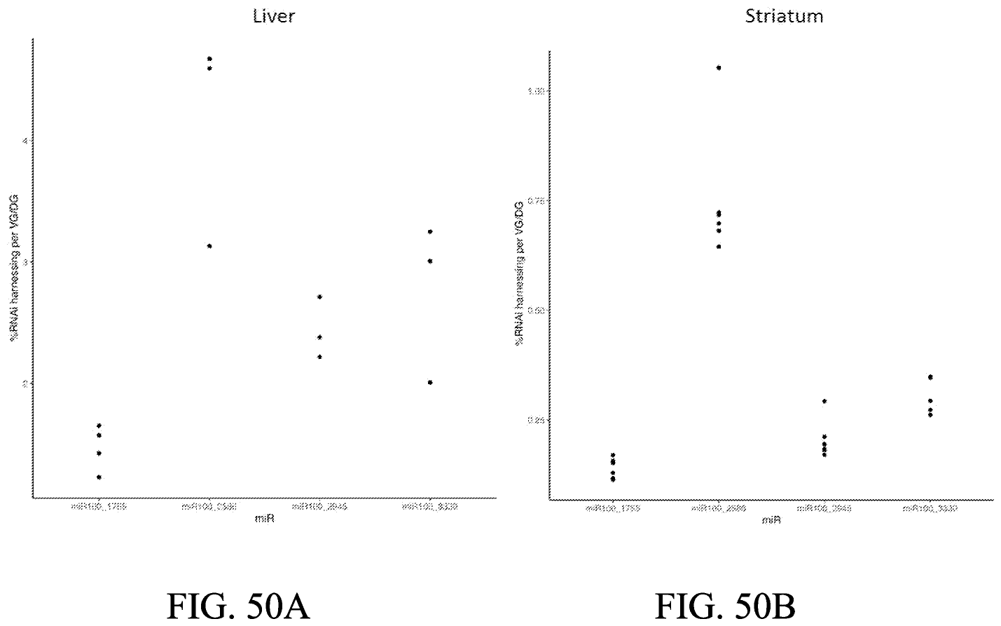

amiRNA expression versus total miRNA expression was assessed in a subset of samples in both liver and striatal punch biopsies. FIG. 50 shows, for each tissue, vector biodistribution-normalized miRNA expression. In both tissues, miR100_1755 has the lowest miRNA expression, followed by miR100_2945, miR100_3330, and lastly miR100_2586.

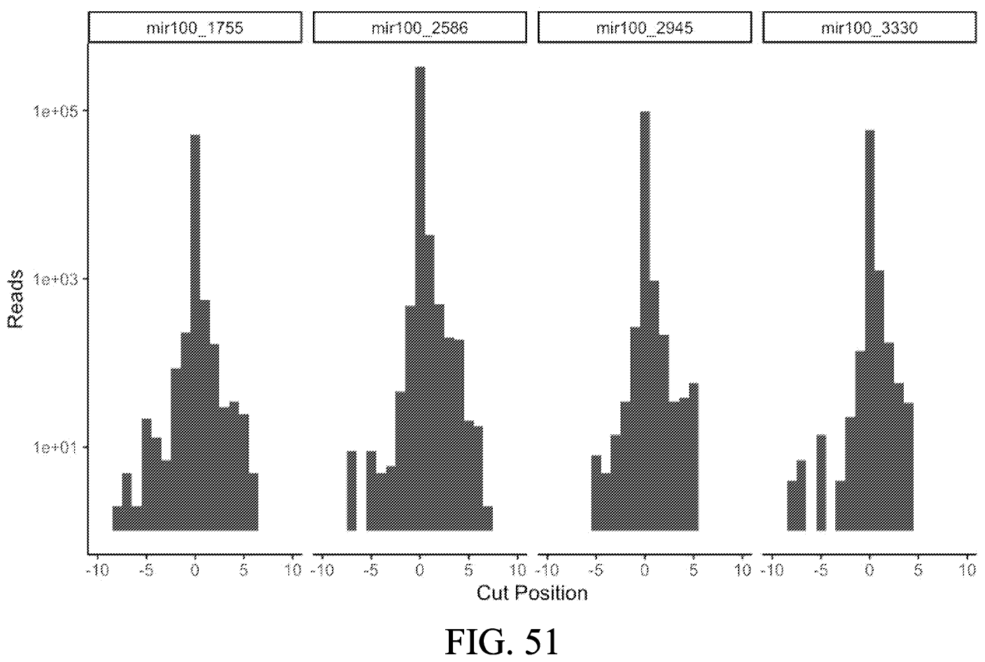

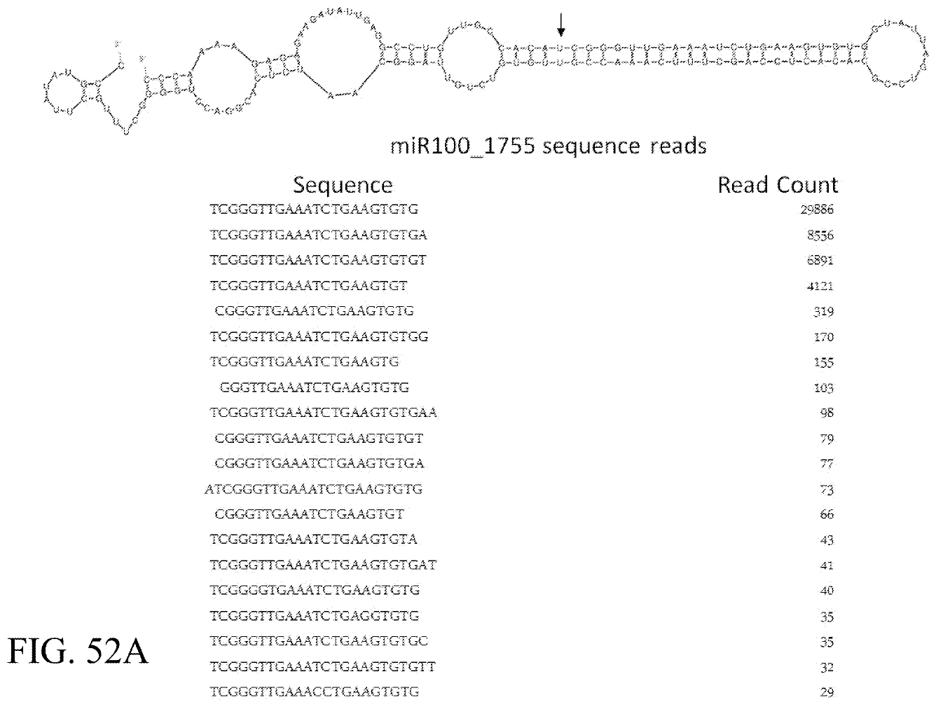

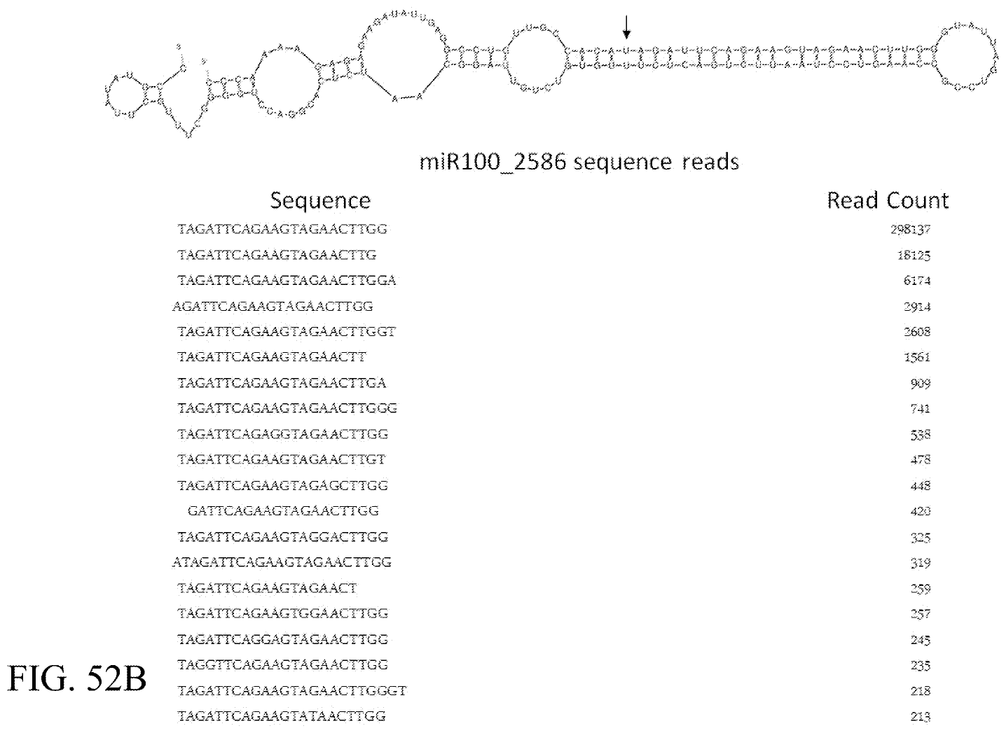

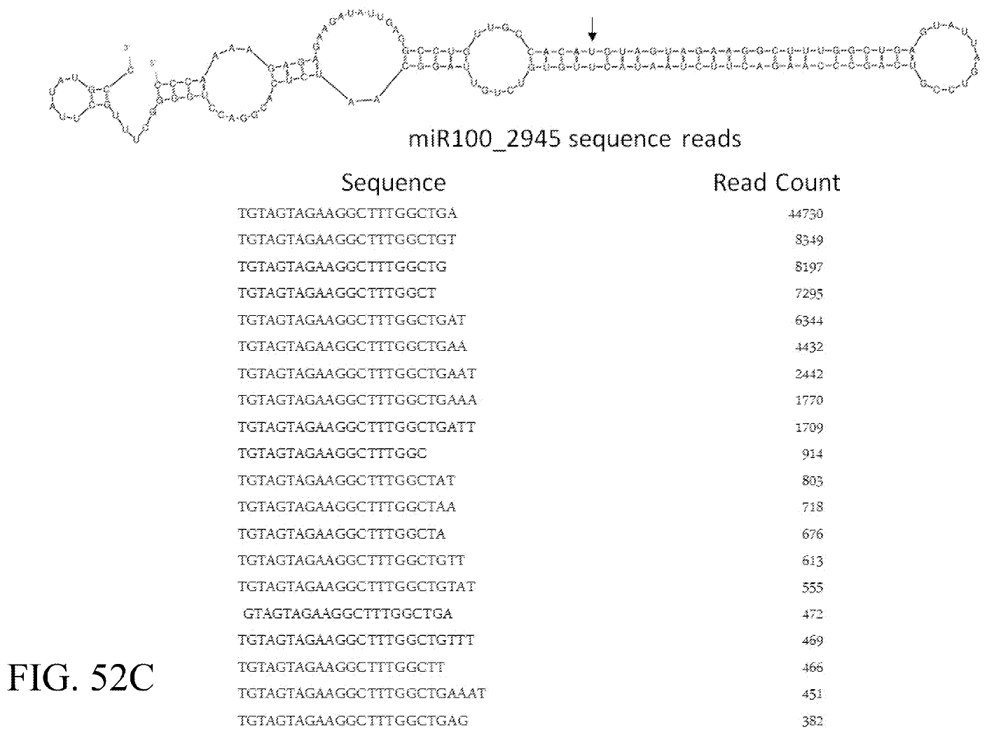

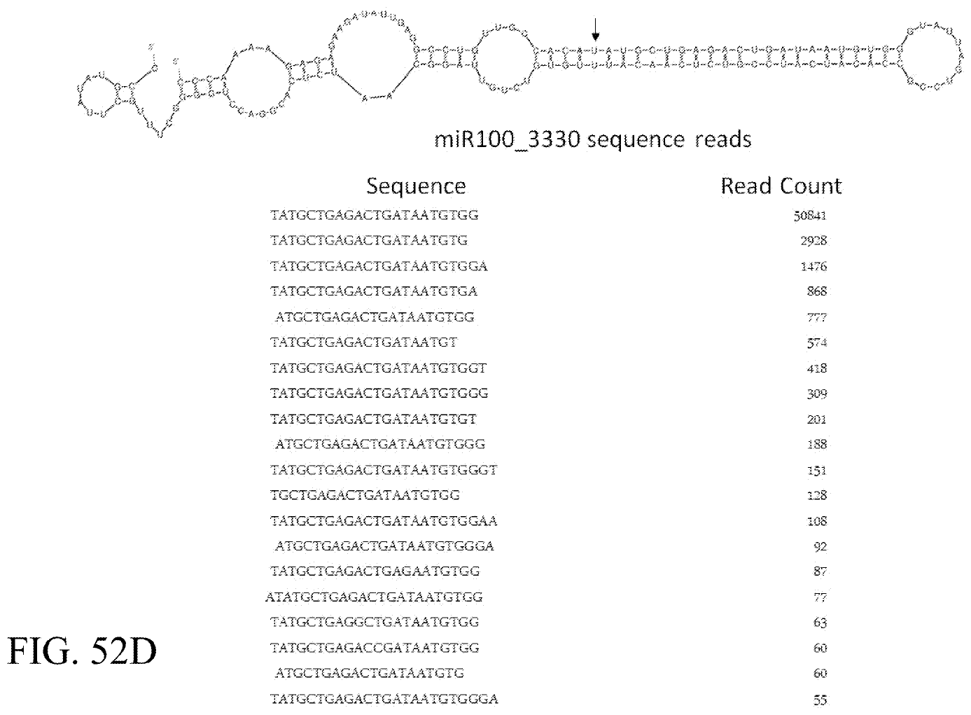

Guide processing precision was also assessed in vivo, by counting reads that initiated at each position of the guide and predicted passenger sequences. FIGS. 51, and 52A-51D shows the count of reads aligning to the miR at the start position, where +0 is the expected typical cut position. Table 31 quantifies the proportion out of all reads (including all guide and passenger strand reads) initiating at the +0 and +1 positions for each amiR. FIGS. 52A-52D show the read counts for the top 20 most common sequences for miR100_1755, miR100_2586, miR100_2945 and miR100_3330. Interestingly, by comparing the observed reads versus the pri-miRNA sequence, post-translational modifications such as 3′ monoadenylation or monouridylation can be noted. Since 3′ mismatches to the target transcript have in some cases been reported to increase the knockdown performance of miRNAs (Becker et al., Molecular Cell (2019) 75:741-755), it may be the case that this 3′ modification of these amiRs may contribute to the high knockdown performance of Atxn2.

TABLE 31
Proportion of amiRNAs Initiating at Position + 0 or + 1

| amiRNA | Proportion cut at + 0 | Proportion cut at + 1 |
|---|---|---|
| mir100_1755 | 97.67 | 1.07 |
| mir100_2586 | 98.51 | 0.99 |
| mir100_2945 | 98.34 | 0.94 |
| mir100_3330 | 97.08 | 2.07 |

Table 32 quantifies the ratio of guide to passenger strand reads. The ratio of reads detected from guide versus passenger strand for all of these miR100 backbone amiRs exceeded 300:1 in vivo. This high processing ratio may reduce the likelihood of off-target effects.

| amiRNA | Ratio guide:passenger |
|---|---|
| mir100_1755 | 399.8 |
| mir100_2586 | 1061.7 |
| mir100_2945 | 651.3 |
| mir100_3330 | 508.3 |

### Methods

For intravenous injection, vector was diluted in PBS with 0.001% PF-68 at 3.21E9vg/10 microliters, and mice were injected via tail vein based on weight (average total dose of 8.5E10 VG). Mice are placed in a restrainer and the tail is swabbed with a sterile alcohol wipe to increase vein visibility. Once a lateral tail vein is located, a 32-gauge insulin syringe is used to administer the solution. 3-weeks post-injection, mice were fasted for 4 hours, blood collected via vena cava and serum processed for AST and ALT analysis. Following PBS perfusion, liver was cut into sections and placed in a homogenizing tube (Precellys, P/N P000933-LYSK0-A) and snap frozen in liquid nitrogen. For tissue homogenization, Buffer RLT supplemented with beta-Mercaptoethanol was added to the sample and a Precellys Cryolys Evolution (Bertin Instruments) with program setting 3×45 s at 5000 rpm with 15 s pauses was performed. Samples tubes were centrifuged at 18,000×g for 3 minutes and a fraction of the homogenate was used for DNA, RNA, and protein purification using the AllPrep DNA/RNA/Protein Mini Kit (Qiagen, P/N 80004) and the other fraction of the homogenate was used for small RNA purification using the miRNeasy Tissue/Cells Advanced Mini Kit (Qiagen, P/N 217604).

For intrastriatal injections, vector was diluted in PBS with 0.001% PF-68 at 7.5E9vg/4 microliters, and mice were injected at coordinates (relative to Bregma) 1.5 mm anterior, +/−1.6 mm lateral, and −4.0 mm ventral with 4 uL per hemisphere (Hamilton P/N 7635-01) over 5 minutes. After 3-weeks post-injection, mice were perfused transcardially with cold PBS and the brain placed in a matrix (CellPoint Scientific, Alto Acrylic 1 mm Mouse Brain Coronal 40-75 gm), and a 2 mm cornal section containing the injection site was excised. Within the coronal section, a 2 mm punch biopsy of both the left and right striatum was collected and placed into separate homogenizing tubes (Precellys, P/N P000933-LYSK0-A) then snap frozen in liquid nitrogen. For tissue homogenization, Buffer RLT supplemented with beta-Mercaptoethanol was added to the sample and homogenization with a Precellys Cryolys Evolution (Bertin Instruments) with program setting 3×45 second at 5000 rpm with 15 second pauses was performed. Samples tubes were centrifuged at 18,000×g for 3 minutes and a fraction of the homogenate was used for DNA, RNA, and protein purification using the AllPrep DNA/RNA/Protein Mini Kit (Qiagen, P/N 80004) and the other fraction of the homogenate was used for small RNA purification using the miRNeasy Tissue/Cells Advanced Mini Kit (Qiagen, P/N 217604).

### Example 9: Pharmacology Study of AAV Vector Expressed amiRNA Targeting ATXN2 in Non-Human Primates

Testing in non-human primates is conducted to establish knockdown of ATXN2 by ATXN2-targeting amiRNA rAAV vectors in tissues relevant to neurodegenerative disease, via clinically relevant routes of administration. Tissues to be assessed include spinal cord ventral horn, motor cortex, and cerebellum, which are relevant to neurodegenerative diseases such as ALS or Spinocerebellar ataxia-2.

In non-human primates, test articles (1×10¹²-1×10¹⁴ vg of amiRNA expressed with a H1 promoter and packaged in AAV9) or vehicle are administered into the cisterna magna by intrathecal cervical (IT-C) catheter. Male and female cynomolgus monkeys (Macaca fascicularis) of approximately 2.5-4 kg body weight, are implanted with an intrathecal cervical catheter for dose administration and sample collection. Test articles are administered (4 animals per test article) comprising a single 2.5 mL dose of vehicle or test article via the implanted intrathecal catheter at a rate of 0.3 mL/minute, followed by 0.1 mL of vehicle to flush the dose from the catheter. At 5 to 26 weeks following the administration, animals are sacrificed, and selected tissues harvested for bioanalytical and histological evaluation. ATXN2 protein and mRNA levels are assessed for suppression after treatment with ATXN2 amiRNA packaged in AAV9 with a H1 promoter, relative to the vehicle group.

### Vector Assessment

Test articles for dosing in non-human primates are assessed by multiple assays. One assessment is analytical ultracentrifugation (AUC) for empty and full capsids and quantification of aggregates. Absorbance scans are collected as the material sediments under the force of a gravitational field. Sample sedimentation profile is monitored in real time during centrifugation, which gives an absolute measurement of molecule size and shape. The distribution movement over time is used to calculate the sedimentation coefficient. Fitting the raw data to the Lamm equation results in a continuous distribution, and area under each peak is proportional to the amount present in solution. Empty capsids are expected to sediment at 65S, partial capsids between 65 and 95S, full capsids at 95S, and aggregates at >110S. Measurements indicating majority full capsids are desirable.

AAV9 capsid ELISA is used to assess intact AAV9 capsids. The capture-antibody detects a conformational epitope that is not present on unassembled capsid proteins. The ADK9 antibody is used as capture and detection antibody in the AAV9 titration ELISA. Assay results are expected to corroborate AUC assessment, by comparing AAV9 capsid ELISA with vector genome titers.

Endotoxin is assessed by Limulus amebocyte lysate (LAL). Detection and quantification of bacterial endotoxins less than 10 EU/mL is desired.

Bioburden is assessed by direct inoculation, and less than 10 CFU/100 mL is desired.

For lot release and stability, an in vitro potency assay for gene therapy product potency is performed. In vitro potency is assessed by amiRNA expression by RT-qPCR, ATXN2 mRNA levels by RT-ddPCR, and ATXN2 protein levels by ATXN2 protein FACS in 2v6.11 or Lec2 cells. Cells may be pre-treated with 1 ug/mL ponasterone A (Invitrogen, H10101), 50 mU/mL neuraminidase (Sigma, N7885), and 2 mM hydroxyurea (Sigma, H8627) prior to transduction. Serial dilutions of vector are used to treat cells in a 96 well format, incubating at 4° C. for 30 min, and then 90 min following application of vector. Plates are then transferred to 37° C. After 2-3 days amiRNA, ATXN2 mRNA, and ATXN2 protein are assessed at each dose.

In vivo potency in some experiments is tested prior to dosing non-human primates and is assessed by single dose (such as 8.5E10 vg/gram) administration of test article intravenously into wild-type C57Bl/6 mice. Liver biopsy is collected, homogenized, and DNA and RNA are extracted by Allprep DNA/RNA/Protein mini kit (Qiagen, 80004) for assessment of vector distribution and ATXN2 mRNA knockdown in liver.

Median Tissue Culture Infectious Dose (TCID50) to assess vector infectivity is performed in HelaRC32 cells. HelaRC32 stably express AAV2 rep and cap genes, and the assay involves serial dilutions of vector in a 96 well plate and co-infection with Adenovirus 5 helper virus, lysing cells, extracting DNA, performing qPCR or ddPCR on the vector genome to assess number of infected cells per well across the dilution range.

### Biodistribution and Pharmacodynamic Activity

Non-human primate brain and spinal cord tissue from rAAV vector and control treated animals are collected by punch biopsy or as slabs at necropsy and snap frozen. Samples are homogenized by addition of Buffer RLT (Qiagen) supplemented with beta-mercaptoethanol. Ceramic bead-based homogenization (Precellys, CK14 2 mL) is performed using 3 cycles of 15 s at 6500 rpm and 10 s break. DNA, RNA and protein are extracted with Allprep DNA/RNA/Protein Mini kit (Qiagen, 80004) and small RNA are extracted with miRNEasy Tissue/Cells Advanced Mini kit (Qiagen, 217604).

For isolation of motor neurons from rAAV dosed non-human primates, spinal cord tissues are frozen in liquid nitrogen at necropsy. Cryosections are generated and stained with ARCTURUS HistoGene Quick H&E Stain for LCM, and motor neurons are dissected from each section with the ARCTURURS XT LCM System. DNA and RNA from LCM samples are extracted with PicoPure kits (Thermo Fisher).

For histological evaluations, non-human primate brain and spinal cord tissue are collected at necropsy and fixed with 10% neutral buffered formalin for 24 hr, transferred to 70% ethanol for 3-10 days and embedded into paraffin blocks. Five-micron sections are cut, mounted onto glass slides, and stained for hematoxylin and eosin for histology, or stained in separate protocols for immunohistochemistry or in-situ hybridization.

Vector biodistribution in tissues from animals dosed with rAAV is assessed by ddPCR. Specifically, primer probes that amplify promoter and/or stuffer regions of the vector are used and compared to primer probes specific to host genome and results are expressed as vector genomes per diploid genome.

To isolate biodistribution in tissue material enriched for motor neurons, vector biodistribution is assessed by ddPCR on DNA isolated from spinal cord neurons captured by laser capture microdissection (LCM). Specifically, primer probes that amplify promoter and/or stuffer regions of the vector are used and compared to host diploid genome and results are expressed as vector genomes per diploid genome. Biodistribution in tissue material enriched for other disease-relevant cell types such as motor cortex, containing motor neurons, and cerebellum, containing Purkinje cells, can be assessed by the same ddPCR method in tissue punches from those brain regions.

To measure ATXN2 knockdown in spinal cord motor neurons, ATXN2 mRNA is assessed by RT-ddPCR in spinal cord neurons captured by laser capture microdissection. Knockdown of ATXN2 mRNA is assessed by comparison of spinal cord neurons in amiRNA treated subjects relative to the vehicle treated group, using the ratio of ATXN2 positive droplets to housekeeping genes (GUSB, B2M, TBP, or others). Significant knockdown of ATXN2 in spinal cord neurons in animals dosed with ATXN2 targeting amiRNAs relative to vehicle dosed animals is desirable.

ATXN2 mRNA in spinal cord neurons, cortical motor neurons, cerebellar purkinje cells and other relevant tissues is also assessed by in situ hybridization (ISH) in tissue sections, and by RT-ddPCR in tissue punches. By in situ hybridization, knockdown of ATXN2 mRNA is semi-quantitatively assessed by comparison of amiRNA treated subjects relative to the vehicle group. Significant knockdown in these tissues is desirable, with reductions in ATXN2 mRNA in spinal and cortical motor neurons particularly relevant for ALS and knockdown in Purkinje cells particularly relevant for SCA2. By RT-ddPCR, knockdown is assessed as described above.

ATXN2 protein in spinal cord neurons, cortical motor neurons, cerebellum, and other brain tissues is assessed by immunohistochemistry. Fixed slides are stained with monoclonal ATXN2 antibody (BD, 611378) or polyclonal ATXN2 antibody (Sigma, HPA018295-100UL) using standard protocols. Immunohistochemistry is used to semi-quantitatively assess knockdown of ATXN2 protein, and significant reduction in ATXN2 levels relative to vehicle treated animals is desirable.

Other assays for the pharmacology of ATXN2 amiRNA vectors dosed via administration into the cerebrospinal fluid in non-human primates that may be conducted include ATXN2 assays using alphaLISA® or Simoa® bead technology; or amiRNA detection assays from tissue or body fluids using miRNA-ISH or miRNA RT-qPCR.

ATXN2 protein in bulk tissue is assessed by alphaLISA. The capture antibody is monoclonal ATXN2 antibody (BD, 611378) and detection antibody is polyclonal ATXN2 antibody (ProteinTech, 21776-1-AP). ATXN2 protein in CSF is assessed by custom ATXN2 Simoa assay (Quanterix).

### REFERENCES

1. Auyeung V C, Ulitsky I, McGeary S E, Bartel D P. Beyond secondary structure: Primary-sequence determinants license Pri-miRNA hairpins for processing. Cell. 2013. doi:10.1016/j.cell.2013.01.031
2. Fang W, Bartel D P. The Menu of Features that Define Primary MicroRNAs and Enable De Novo Design of MicroRNA Genes. Mol Cell. 2015. doi:10.1016/j.molcel.2015.08.015
3. Fowler D K, Williams C, Gerritsen A T, Washbourne P. Improved knockdown from artificial microRNAs in an enhanced miR-155 backbone: A designer's guide to potent multi-target RNAi. Nucleic Acids Res. 2015. doi:10.1093/nar/gkv1246
4. Fellmann C, Hoffmann T, Sridhar V, et al. An optimized microRNA backbone for effective single-copy RNAi. Cell Rep. 2013. doi:10.1016/j.celrep.2013.11.020
5. Kozomara A, Birgaoanu M, Griffiths-Jones S. MiRBase: From microRNA sequences to function. Nucleic Acids Res. 2019. doi:10.1093/nar/gky 1141
6. Watanabe C, Cuellar T L, Haley B. Quantitative evaluation of first, second, and third generation hairpin systems reveals the limit of mammalian vector-based RNAi. RNA Biol. 2016. doi:10.1080/15476286.2015.1128062
7. Zuker M. Mfold web server for nucleic acid folding and hybridization prediction. Nucleic Acids Res. 2003. doi:10.1093/nar/gkg595
8. Chung K H, Hart C C, Al-Bassam S, et al. Polycistronic RNA polymerase II expression vectors for RNA interference based on BIC/miR-155. Nucleic Acids Res. 2006. doi:10.1093/nar/gkl143
9. Lebbink R J, Lowe M, Chan T, Khine H, Wang X, McManus M T. Polymerase II promoter strength determines efficacy of microrna adapted shRNAs. PLoS One. 2011. doi:10.1371/journal.pone.0026213
10. Pfister E L, Chase K O, Sun H, et al. Safe and Efficient Silencing with a Pol II, but Not a Pol lII, Promoter Expressing an Artificial miRNA Targeting Human Huntingtin. Mol Ther—Nucleic Acids. 2017. doi:10.1016/j.omtn.2017.04.011
11. Dirren E, Aebischer J, Rochat C, Towne C, Schneider B L, Aebischer P. SOD1 silencing in motoneurons or glia rescues neuromuscular function in ALS mice. Ann Clin Transl Neurol. 2015. doi:10.1002/acn3.162
12. Pfister E L, Dinardo N, Mondo E, et al. Artificial miRNAs Reduce Human Mutant Huntingtin Throughout the Striatum in a Transgenic Sheep Model of Huntington's Disease. Hum Gene Ther. 2018. doi:10.1089/hum.2017.199
13. Borel F, Gernoux G, Cardozo B, et al. Therapeutic rAAVrh10 Mediated SOD1 Silencing in Adult SOD1G93A Mice and Nonhuman Primates. Hum Gene Ther. 2016. doi:10.1089/hum.2015.122
14. Gao Z, Herrera-Carrillo E, Berkhout B. RNA Polymerase II Activity of Type 3 Pol III Promoters. Mol Ther—Nucleic Acids. 2018. doi:10.1016/j.omtn.2018.05.001
15. Kampmann M, Horlbeck M A, Chen Y, et al. Next-generation libraries for robust RNA interference-based genome-wide screens. Proc Natl Acad Sci USA. 2015. doi:10.1073/pnas.1508821112
16. Gu S, Jin L, Zhang Y, et al. The loop position of shRNAs and pre-miRNAs is critical for the accuracy of dicer processing in vivo. Cell. 2012. doi:10.1016/j.cell.2012.09.042
17. Chang K, Marran K, Valentine A, Hannon G J. Generation of transgenic Drosophila expressing shRNAs in the miR-1 backbone. Cold Spring Harb Protoc. 2014. doi:10.1101/pdb.prot080762
18. Hoye M L, Koval E D, Wegener A J, et al. MicroRNA profiling reveals marker of motor neuron disease in ALS models. J Neurosci. 2017. doi:10.1523/JNEUROSCI.3582-16.2017
19. Ludwig N, Leidinger P, Becker K, et al. Distribution of miRNA expression across human tissues. Nucleic Acids Res. 2016. doi:10.1093/nar/gkw116
20. Otaegi G, Pollock A, Hong J, Sun T. MicroRNA miR-9 modifies motor neuron columns by a tuning regulation of FoxP1 levels in developing spinal cords. J Neurosci. 2011. doi:10.1523/JNEUROSCI.4330-10.2011
21. Morgens D W, Deans R M, Li A, Bassik M C. Systematic comparison of CRISPR/Cas9 and RNAi screens for essential genes. Nat Biotechnol. 2016. doi:10.1038/nbt.3567
22. Hart T, Chandrashekhar M, Aregger M, et al. High-Resolution CRISPR Screens Reveal Fitness Genes and Genotype-Specific Cancer Liabilities. Cell. 2015. doi:10.1016/j.cell.2015.11.015
23. Leonetti M D, Sekine S, Kamiyama D, Weissman J S, Huang B. A scalable strategy for high-throughput GFP tagging of endogenous human proteins. Proc Natl Acad Sci USA. 2016. doi:10.1073/pnas.1606731113
24. Sack L M, Davoli T, Xu Q, Li M Z, Elledge S J. Sources of error in mammalian genetic screens. G3 Genes, Genomes, Genet. 2016. doi:10.1534/g3.116.030973
25. Hawkins J A, Jones S K, Finkelstein I J, Press W H. Indel-correcting DNA barcodes for high-throughput sequencing. Proc Natl Acad Sci USA. 2018. doi:10.1073/pnas.1802640115
26. Morgens D W, Wainberg M, Boyle E A, et al. Genome-scale measurement of off-target activity using Cas9 toxicity in high-throughput screens. Nat Commun. 2017. doi:10.1038/ncomms15178
27. Hsiau T, Maures T, Waite K, et al. Inference of CRISPR Edits from Sanger Trace Data. bioRxiv. 2018. doi:10.1101/251082

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including but not limited to U.S. Provisional Application No. 62/971,873 filed on Feb. 7, 2020, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. An isolated nucleic acid comprising an expression construct encoding an inhibitory nucleic acid that inhibits expression or activity of ATXN2, wherein the inhibitory nucleic acid comprises a guide strand sequence comprising the nucleic acid sequence set forth in any one of SEQ ID NOS: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 1176-1288, 1811-1827, 2015, 2065, 2083, 2152, 2203, and 2209.

2. (canceled)

3. The isolated nucleic acid molecule of claim 1, wherein the inhibitory nucleic acid is a siRNA duplex, shRNA, miRNA, or dsRNA.

4. The isolated nucleic acid molecule of claim 1, wherein the inhibitory nucleic acid further comprises a passenger strand sequence, optionally wherein the passenger strand sequence is selected from Tables 1, 19, 23, and 24, or a passenger strand sequence selected from Tables 1, 19, 23, and 24 and having 1-10 insertions, deletions, substitutions, mismatches, wobbles, or any combination thereof.

5. The isolated nucleic acid molecule of claim 4, wherein the inhibitory nucleic acid is an artificial miRNA, wherein the guide strand sequence and passenger strand sequence are contained within a miRNA backbone sequence.

6. (canceled)

7. The isolated nucleic acid molecule of claim 5, wherein the miRNA backbone sequence is a miR-155 backbone sequence, a miR-155E backbone sequence, a miR-155M backbone sequence, a miR1-1 backbone sequence, a miR-1-1_M backbone sequence, a miR-100 backbone sequence, a miR-100_M backbone sequence, a miR-190a backbone sequence, a miR-190a_M backbone sequence, a miR-124 backbone sequence, a miR-124_M backbone sequence, a miR-132 backbone sequence, a miR-9 backbone sequence, a miR-138-2 backbone sequence, a miR-122 backbone sequence, a miR-122_M backbone sequence, a miR-130a backbone sequence, a miR-16-2 backbone sequence, a miR-128 backbone sequence, a miR-144 backbone sequence, a miR-451a backbone sequence, or a miR-223 backbone sequence.

8. (canceled)

9. (canceled)

10. (canceled)

11. The isolated nucleic acid molecule of claim 1, wherein the inhibitory nucleic acid is a miRNA comprising the nucleic acid sequence set forth in any one of SEQ ID NOS: 443-490, 1109-1111, 1114, 1121-1168, 1405-1520, 1908-2007, 2011, 2017, 2021, 2025, 2027, 2031, 2035, 2039, 2041, 2045, 2049, 2053, 2057, 2061, 2067, 2071, 2075, 2079, 2085, 2089, 2093, 2097, 2101, 2105, 2109, 2113, 2117, 2120, 2124, 2128, 2132, 2136, 2140, 2144, 2148, 2154, 2158, 2162, 2166, 2170, 2174, 2176, 2180, 2182, 2184, 2187, 2189, 2191, 2193, 2195, 2197, 2199, 2205, 2211, 2261, 2263, 2265, and 2267.

12. (canceled)

13. The isolated nucleic acid molecule of claim 1, wherein the nucleic acid sequence encoding the inhibitory nucleic acid is located in an untranslated region of the expression construct.

14. (canceled)

15. The isolated nucleic acid molecule of claim 1, further comprising a promoter operably linked to the nucleic acid sequence encoding the inhibitory nucleic acid.

16. The isolated nucleic acid molecule of claim 15, wherein the promoter is a RNA pol III promoter, U6 promoter, H1 promoter, a chicken-beta actin (CBA) promoter, a CAG promoter, a H1 promoter, a CD68 promoter, a human synapsin promoter, or a JeT promoter.

17. The isolated nucleic acid molecule of claim 15, wherein the promoter is an H1 promoter comprising nucleotides 113-203 of SEQ ID NO:1522, nucleotides 1798-1888 of SEQ ID NO:1521, nucleotides 244-343 of SEQ ID NO:2257, or nucleotides 113-343 of SEQ ID NO:2257.

18. The isolated nucleic acid molecule of claim 1, wherein the expression construct is flanked by a 5′ adeno-associated virus (AAV) inverted terminal repeat (ITR) sequence and a 3′ AAV ITR sequence, or variants thereof.

19. The isolated nucleic acid molecule of claim 18, wherein one of the ITR sequences lacks a functional terminal resolution site.

20. The isolated nucleic acid molecule of claim 18, wherein the 5′ and 3′ ITRs are derived from an AAV serotype selected from the group consisting of: AAV1, AAV2, AAV5, AAV6, AAV6.2, AAV7, AAV8, AAV9, AAVRh10, AAV11, and variants thereof.

21. The isolated nucleic acid molecule of claim 18, wherein the 5′ ITR comprises nucleotides 1-106 of SEQ ID NO:2257 and the 3′ ITR comprises nucleotides 2192-2358 of SEQ ID NO:2257.

22. A vector comprising the isolated nucleic acid molecule of claim 1.

23. The vector of claim 16, wherein the vector is a plasmid or viral vector.

24. The vector of claim 23, wherein the viral vector is a recombinant adeno-associated virus (rAAV) vector or a Baculovirus vector.

25. The vector of claim 24, wherein the vector is a self-complementary rAAV vector.

26. The vector of claim 24, wherein the rAAV vector further comprises a stuffer sequence.

27. (canceled)

28. (canceled)

29. A recombinant adeno-associated (rAAV) particle comprising the isolated nucleic acid molecule of claim 1.

30. The rAAV particle of claim 29, wherein the rAAV particle comprises a capsid protein.

31. The rAAV particle of claim 30, wherein the capsid protein is capable of crossing the blood-brain barrier.

32. The rAAV particle of claim 30, wherein the capsid protein is an AAV9 capsid protein or AAVrh.10 capsid protein.

33. (canceled)

34. A pharmaceutical composition comprising the isolated nucleic acid molecule of claim 1, and optionally a pharmaceutically acceptable carrier.

35. A host cell comprising the isolated nucleic acid molecule of claim 1.

36. A method for treating a subject having or suspected of having a neurodegenerative disease, the method comprising administering to the subject the isolated nucleic acid molecule of claim 1.

37. The method of claim 36, wherein the administration comprises direct injection to the CNS of the subject.

38. The method of claim 37, wherein the direct injection is intracerebral injection, intraparenchymal injection, intrathecal injection, intrastriatal injection, subpial injection, direct injection to the cerebrospinal fluid (CSF) of the subject, intracistemal injection, intraventricular injection, intralumbar injection, or any combination thereof.

39. (canceled)

40. The method of claim 36, wherein the subject is characterized as having an ATXN2 allele having at least 22 CAG trinucleotide repeats, optionally wherein the ATXN2 allele has at least 24 CAG trinucleotide repeats, at least 27 CAG trinucleotide repeats, at least 30 CAG trinucleotide repeats, or at least 33 or more CAG trinucleotide repeats.

41. The method of claim 36, wherein the neurodegenerative disease is spinocerebellar ataxia-2, amyotrophic lateral sclerosis, frontotemporal dementia, primary lateral sclerosis, progressive muscular atrophy, limbic-predominant age-related TDP-43 encephalopathy, chronic traumatic encephalopathy, dementia with Lewy bodies, corticobasal degeneration, progressive supranuclear palsy (PSP), dementia Parkinsonism ALS complex of guam (G-PDC), Pick's disease, hippocampal sclerosis, Huntington's disease, Parkinson's disease, or Alzheimer's disease.

42. A method of inhibiting ATXN2 expression in a cell, the method comprising delivering to the cell the isolated nucleic acid of claim 1.

43. (canceled)

44. (canceled)

45. (canceled)

46. (canceled)

47. (canceled)

48. (canceled)

49. A method of inhibiting ATXN2 expression in the central nervous system of a subject, the method comprising administering to the subject the isolated nucleic acid of claim 1.

50. (canceled)

51. (canceled)

52. (canceled)

53. (canceled)

54. An artificial miRNA comprising a guide strand sequence and a passenger strand sequence, wherein the guide strand sequence comprises any one of SEQ ID NOS: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 1176-1288, 1811-1827, 2015, 2065, 2083, 2152, 2203, and 2209.

55. (canceled)

56. The artificial miRNA of claim 54, wherein the guide strand sequence and passenger strand sequence are contained within a miR backbone sequence.

57. The artificial miRNA of claim 56, wherein the miR backbone sequence is a miR-155 backbone sequence, a miR-155E backbone sequence, a miR-155M backbone sequence, a miR1-1 backbone sequence, a miR-1-1_M backbone sequence, a miR-16-2 backbone sequence, a miR-100 backbone sequence, a miR-100_M backbone sequence, a miR-190a backbone sequence, a miR-190a_M backbone sequence, a miR-124 backbone sequence, a miR-124_M backbone sequence, a miR-132 backbone sequence, a miR-9 backbone sequence, a miR-138-2 backbone sequence, a miR-122 backbone sequence, a miR-122_M backbone sequence, a miR-130a backbone sequence, or a miR-128 backbone sequence, a miR-144 backbone sequence, a miR-451a backbone sequence, or a miR-223 backbone sequence.

58. (canceled)

59. (canceled)

60. (canceled)

61. The artificial miRNA of claim 54, wherein the artificial miRNA comprises the sequence as set forth in any one of SEQ ID NOS: 443-490, 1109-1111, 1114, 1121-1168, 1405-1520, 1908-2007, 2011, 2017, 2021, 2025, 2027, 2031, 2035, 2039, 2041, 2045, 2049, 2053, 2057, 2061, 2067, 2071, 2075, 2079, 2085, 2089, 2093, 2097, 2101, 2105, 2109, 2113, 2117, 2120, 2124, 2128, 2132, 2136, 2140, 2144, 2148, 2154, 2158, 2162, 2166, 2170, 2174, 2176, 2180, 2182, 2184, 2187, 2189, 2191, 2193, 2195, 2197, 2199, 2205, 2211, 2261, 2263, 2265, and 2267.

62. An isolated RNA duplex comprising a guide strand sequence and a passenger strand sequence, wherein the guide strand sequence comprises the nucleic acid sequence set forth in any one of SEQ ID NOS: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, and 1176-1288, 1811-1827, 2015, 2065, 2083, 2152, 2203, and 2209, optionally wherein the guide strand sequence and passenger strand sequence are linked by a loop region to form a hairpin structure comprising a duplex structure and a loop region.

63. (canceled)
